(19) United States
(12) Patent Application Publication
(10) Pub. No.: US 2019/0328706 A1
(43) Pub. Date: Oct. 31, 2019

(54) COMPOSITIONS COMPRISING AN INTEGRIN INHIBITOR AND AGENTS WHICH INTERACT WITH A CHEMOKINE AND METHODS OF USE THEREOF

(71) Applicants: Saint Louis University, St. Louis, MO (US); Washington University, St. Louis, MO (US)

(72) Inventors: Peter Ruminski, Wildwood, MO (US); Marvin Meyers, Wentzville, MO (US); Richard F. Heier, Columbia, IL (US); Michael Rettig, St. Louis, MO (US); John DiPersio, St. Louis, MO (US); Darja Karpova, St. Louis, MO (US)

(21) Appl. No.: 16/401,950

(22) Filed: May 2, 2019

Related U.S. Application Data
(63) Continuation-in-part of application No. PCT/US2017/059777, filed on Nov. 2, 2017.
Application No. PCT/US2017/059777 is a continuation-in-part of application No. PCT/US2017/059733, filed on Nov. 2, 2017.
Application No. PCT/US2017/059733 is a continuation-in-part of application No. PCT/US2017/059770, filed on Nov. 2, 2017.
(60) Provisional application No. 62/416,555, filed on Nov. 2, 2016.
(60) Provisional application No. 62/416,462, filed on Nov. 2, 2016.
(60) Provisional application No. 62/430,167, filed on Dec. 5, 2016.
(60) Provisional application No. 62/554,806, filed on Sep. 6, 2017.
(60) Provisional application No. 62/416,530, filed on Nov. 2, 2016.
(60) Provisional application No. 62/554,791, filed on Sep. 6, 2017.
(60) Provisional application No. 62/558,045, filed on Sep. 13, 2017.
(60) Provisional application No. 62/416,462, filed on Nov. 2, 2016.
(60) Provisional application No. 62/430,167, filed on Dec. 5, 2016.

(51) Int. Cl. A61K 31/397; A61K 31/135; C07D 207/48; C07D 205/04; C07C 233/87; A61K 31/197; A61K 31/40

(52) U.S. Cl. CPC A61K 31/397; A61K 31/135; C07D 207/48; A61K 31/40; C07C 233/87; A61K 31/197; C07D 205/04

(57) ABSTRACT

The present disclosure provides therapeutic agents including those of the formula:

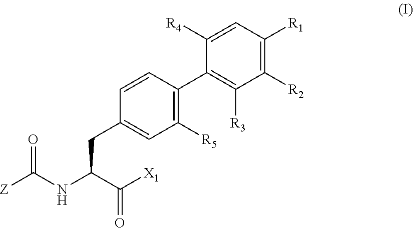

wherein the variables are defined herein. Also provided are pharmaceutical compositions, kits and articles of manufacture comprising such therapeutic agents. Methods of using the therapeutic agents are also provided. The present disclosure further provides combinations of a VLA-4 inhibitor and an agent which interacts with a chemokine receptor, and methods of use thereof. In some embodiments, the disclosed combinations may be used in a method of mobilizing hematopoietic stem cells. In some embodiments, the disclosed methods may be used in the treatment of a condition which requires the collection of hematopoietic stem cells for transfusions or in chemotherapy. The present disclosure further provides methods of treating a patient comprising administering an agent which interacts with a chemokine such as G-CSF, plerixafor, or Gro-β and VLA-4 inhibitors.

### CROSS REFERENCE TO RELATED APPLICATIONS

This application claims the benefit of International Patent Application No. PCT/US2017/059770, filed Nov. 2, 2017; and the benefit of International Patent Application No. PCT/US2017/059777, filed Nov. 2, 2017; and the benefit of International Patent Application No. PCT/US2017/059733, filed Nov. 2, 2017 which claims the benefit of U.S. Provisional Application No. 62/416,555, filed on Nov. 2, 2016, and the benefit of U.S. Provisional Application No. 62/416,462, filed on Nov. 2, 2016, and the benefit of U.S. Provisional Application No. 62/430,167, filed on Dec. 5, 2016, and the benefit of U.S. Provisional Application No. 62/554,806, filed on Sep. 6, 2017, the entire contents of each are hereby incorporated by reference.

### GOVERNMENTAL RIGHTS

This invention was made with government support under CA152329 awarded by the National Institute of Health. The government has certain rights in the invention.

### FIELD OF THE TECHNOLOGY

The present disclosure relates to the fields of pharmaceuticals, medicine and cell biology. More specifically, it relates to pharmaceutical agents such as VLA-4 inhibitor and a first and second agent which interacts with a chemokine used in combination to enhance the collection of hematopoietic stem cells.

### BACKGROUND

Hematopoietic stem cell transplantation (HSCT) is the major curative therapy available for many hematological diseases including hematological cancers. In this technique, HSCT is used to facilitate repopulation of healthy bone marrow and immune system cells after a high-dose chemotherapy treatment for cancers including but not limited to Hodgkin's and non-Hodgkin's lymphoma, multiple myeloma, and leukemia. In order to facilitate transplantation when the cells are need, hematopoietic stem/progenitor cells (HSPCs) are collected from the patient's blood, harvested, frozen and then stored while the patient receives high-dose chemotherapy and/or radiation therapy. In order to achieve a successful transplantation, an intravenous infusion of a minimum number of 2×10⁶ CD34+ stem cells/kg body weight is often needed; however, a dose of 5×10⁶ CD34+ cells/kg is considered preferable for early and long term multilineage engraftment.

Currently, the stem cells for hematopoietic stem cell transplants are often harvested from peripheral blood. Due to the low amount of these cells in circulating peripheral blood, the stem cells often must be stimulated to increase the quantity in the peripheral blood, a process which can take almost a week. Even then, the collection is still done over several days to achieve sufficient concentrations of the stem cells for transplantation. This greatly increases the cost of the transplant and results in a significant burden on the patient. Currently, cytokines, such as granulocyte-colony forming unit (G-CSF), and immunostimulants, such as plerixafor, are used to increase the amount of hematopoietic stem cells in the peripheral blood but a single agent often results in insufficient mobilization of stem cells. Additional methods of harvesting hematopoietic stem cells have been developed which involve combining G-CSF with multiple other agents such as plerixafor or another cytokine. Unfortunately, even these combined therapies often fail to increase the concentrations to sufficient levels for transplantation in many patients even with multiple days of apheresis. Furthermore, several of these agents, like plerixafor, are extremely expensive adding over $25,000 per patient relative to using G-CSF alone. Therefore, a need remains for better methods to harvest hematopoietic stem cells.

### SUMMARY

The present disclosure provides compounds which are VLA-4 antagonists (i.e. inhibitors), pharmaceutical compositions, methods for their manufacture, and methods for their use.

The present disclosure provides methods using a compound which is a VLA-4 antagonist in combination with an agent which interacts with a chemokine receptor (such as a CXCR2 agonist) including methods of use and methods of treatment therewith. Also, provided herein are compositions comprising these two compounds.

The present disclosure provides methods using a compound which includes VLA-4 antagonists in combination with a first and second agent which interacts with a chemokine (such as CXCR2 agonist and a CXCR4 inhibitor) including methods of use and methods of treatment therewith. Also, provided herein are compositions comprising these three compounds.

Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description. Note that simply because a particular compound is ascribed to one particular generic formula does not mean that it cannot also belong to another generic formula.

### BRIEF DESCRIPTION OF THE FIGURES

The application file contains at least one drawing executed in color. Copies of this patent application publication with color drawing(s) will be provided by the Office upon request and payment of the necessary fee.

The following drawings form part of the present specification and are included to further demonstrate certain aspects of the present disclosure. The disclosure may be better understood by reference to one or more of these drawings in combination with the detailed description of specific embodiments presented herein.

FIG. 1 shows CFU-C mobilization. DBA/2 mice were treated SC with 3 mg/kg of inhibitors and the kinetics of CFU-C mobilization to the blood was measured.

FIG. 2 shows the results of the colony forming cell (CFC) assay. The top graph shows the number of colonies formed as a function of time while the bottom graph shows the number of CFC units at a specific time point for each mouse. These results show the results for Example Compound 1610 (light gray) and Example Compound 1611 (dark gray).

FIG. 3 depicts a graph showing CFC mobilization by firategrast. B6 mice were treated with AMD3100 (5 mg/kg sc), firategrast (100 mg/kg IV), and G-CSF (250 mg/kg/d×5 days) alone or in combination. *P≤0.05, **P≤0.01, ***P≤0.001.

FIG. 4 depicts a graph showing CFC mobilization by firategrast+ Groβt. DBA/2 mice were treated with firategrast (100 mg/kg IV) and Groβt (2.5 mg/kg) alone or in combination. n=5. **P≤0.01, ***P≤0.001

FIG. 5 depicts flow cytometry data showing that VLA-4 inhibitors result in a decrease sVCAM-1-PE fluorescence, indicating a decrease in VCAM binding. BIO5192 and Firategrast were used as positive controls. Also depicted is a graph showing decreasing sVCAM-1-PE signal with increasing inhibitor concentrations as a percentage of the control fluorescence.

FIG. 6 depicts flow cytometry data showing that a set of different VLA-4 inhibitors result in a decrease sVCAM-1-PE fluorescence, indicating a decrease in VCAM binding. BIO5192 was used as a positive control.

FIG. 7 depicts a graph showing decreasing sVCAM-1-PE signal with increasing inhibitor concentrations of a variety of potential VLA-4 inhibitors, as a percentage of the control fluorescence from flow cytometry assays. BIO5192 was used as a positive control.

FIG. 8A depicts flow cytometry data showing that a set of VLA-4 inhibitors result in a decrease sVCAM-1-PE fluorescence, indicating a decrease in VCAM binding. BIO5192 and Firategrast and Zaurategrast were used as positive controls. FIG. 8B depicts a graph showing decreasing sVCAM-1-PE signal with increasing inhibitor concentrations as a percentage of the control fluorescence.

FIG. 9A depicts flow cytometry data showing that a different set of VLA-4 inhibitors result in a decrease sVCAM-1-PE fluorescence, indicating a decrease in VCAM binding. BIO5192 and Firategrast were used as positive controls.

FIG. 9B depicts a graph showing decreasing percentage of cells which are sVCAM positive with increasing inhibitor concentrations, as measured by sVCAM-1-PE signal.

FIG. 10A depicts flow cytometry data showing that a different set of VLA-4 inhibitors result in a decrease sVCAM-1-PE fluorescence, indicating a decrease in VCAM binding. BIO5192 and Firategrast were used as positive controls.

FIG. 10B depicts a graph showing decreasing percentage of cells which are sVCAM positive with increasing inhibitor concentrations, as measured by sVCAM-1-PE signal. And further depicts a graph showing decreasing median fluorescence intensity of sVCAM-1-PE with increasing inhibitor concentrations.

FIG. 11A depicts flow cytometry data showing that a different set of VLA-4 inhibitors result in a decrease sVCAM-1-PE fluorescence, indicating a decrease in VCAM binding. BIO5192 and Firategrast were used as positive controls.

FIG. 11B depicts a graph showing decreasing percentage of cells which are sVCAM positive with increasing inhibitor concentrations, as measured by sVCAM-1-PE signal. And further depicts a graph showing decreasing median fluorescence intensity of sVCAM-1-PE with increasing inhibitor concentrations.

FIG. 12 depicts the results of a VCAM cytometric assay using increasing concentrations of the indicated inhibitors, conducted with G2 ALL cells or 5TGMI cells. Percentage of cells which are positive for sVCAM-1 binding is depicted as compared to control cells.

FIG. 13A and FIG. 13B show HSPC mobilization and transplantation. (FIG. 13A) DBA/2 mice (n=3/cohort) were treated and blood CFC and LSK numbers were determined 0.5 h post-injection. (FIG. 13B) B6 (CD45.2+) mice were treated and blood was collected 0.5 h post-injection. Mobilized blood (pooled from n=4/cohort) were co-transplanted with 2.5×105 competitor BM (CD45.1/2+) into lethally irradiated recipients (CD45.1+, n=10/mobilizing agent). The frequency of repopulating units (RU) was calculated based on the contribution within the B-cell lymphoid and myeloid fraction 20 weeks after transplant. *P≤0.05, **P≤0.01.

FIG. 14A shows the results of the colony forming cell (CFC) assay with the integrin antagonist administered in combination with Groβt. The top graph shows the number of colonies formed as a function of time while the bottom graph shows the number of CFC units at a specific time point for each mouse. These results show the results for Example Compound 1610 (light gray) and Example Compound 1611 (dark gray). FIG. 14B shows the results of the colony forming cell (CFC) assay with only the VLA-4 inhibitor. The top graph shows the number of colonies formed as a function of time while the bottom graph shows the number of CFC units at a specific time point for each mouse. These results show the results for Example Compound 1610 (light gray) and Example Compound 1611 (dark gray).

FIG. 15 shows the effects of blocking VLA4 with CXCR2 stimulation results in the mobilization of both LSK and CFU-C cells in DBA2/J mice. n=3 ** P≤0.01 **P≤0.05 2-way ANOVA with Sidak post-hoc correction.

FIG. 16 shows the results of the colony forming cell (CFC) assay with the VLA-4 inhibitor administered in alone, or in combination with Groβt. The left shows the number of colonies formed as a function of time with only VLA-4 inhibitor, while the right graph shows the number of colonies formed with administration of both the VLA-4 inhibitor and Groβt.

FIG. 17 shows that the indicated VLA-4 inhibitors are capable of mobilizing LSK cells.

FIGS. 18A-18C demonstrate the effectiveness of treatment with other VLA-inhibitors and Groβt in mobilizing stem cells. Mice injected with the VLA-4 inhibitor and Groβt had peripheral blood collected at 0.25, 2, and 4 hours post injection with compounds shows from left to right as listed in the legend. The blood aliquots were analyzed by flow cytometry to separate LSK cells and LSK-SLAM cells and further analyzed by Hemavet. FIG. 18A shows Hemavet analysis of total WBCs indicating significantly greater mobilization of stem cells after 0.25 or 2 hours in mice treated with both VLA-4 inhibitors and Groβt than untreated cells, or cells treated only with Groβt. The total WBCs returned to levels similar to baseline after 4 hours.

FIG. 18B and FIG. 18C show that both LSK cells and LSK-SLAM cells were significantly mobilized by treatment with the combination of VLA-4 inhibitor and Groβt.

FIG. 19A and FIG. 19B show pharmacokinetics (n=3) (FIG. 19A) and pharmacodynamics (n=4-6) (FIG. 19B) of different methods of administering the agents in DBA2/J mice.

FIG. 20A and FIG. 20B show the results of repopulation assay show that the mobilized blood results in functional HPSC from either primary (n=8-18) or secondary recipients (n=5-10) in BALB/C mice. FIG. 9A shows the study design.

FIG. 21A-FIG. 21C show the results of order of administration of the agents. FIG. 21A shows that similar results were obtained when the VLA-4 inhibitor was administered before Groβt. Similar results were obtained for the opposite combination wherein Groβt was administered before the VLA-4 inhibitor (FIG. 21B). *** P<0.001 The highest CFU-C was seen when the agents were administered simultaneously as shown in FIG. 21C. *** P<0.001 ** P<0.001 t-test with Holm-Sidak correlation. n=4-5 and conducted in BALB/C mice.

FIG. 22A-FIG. 22C show that compositions containing Groβt resulted in the induction of MMP9 proteolytic activity in blood (FIG. 22A) in BALB/C mice (n=3). Myeloid cells express both CXCR2 and MMP9 (FIG. 22B) and may serve as a key cell type for HSPC mobilization in BALB/C mice. (flow cytometry n=4; real time PCR n=3-9). FIG. 22C shows that GR1+ myeloid cells are required to mobilize HSPCs in BALB/C mice. n=7-9 * P<0.05.

FIG. 23 shows the combination of a VLA-4 inhibitor and Groβt results in similar mobilization regardless of the presence of other factors which result in poor stem cell mobilization such as diabetes.

FIG. 24A and FIG. 24B show testing of further combinations of VLA-4 inhibitors with CXCR4 inhibitors and CXCR2 agonists with compounds shows from left to right as listed in the legend. FIG. 24A shows that significantly more stem cells were mobilized following treatment with the VLA-4 inhibitor and any of the listed combination therapies than with CXCR2 agonist alone or the CXCR4 inhibitor alone.

FIG. 24B shows a spike in CFUs for the treatment with just the VLA-4 inhibitor and combined VLA-4 inhibitor and CXCR2 agonist treatment.

FIG. 25 depicts a graph showing CFC mobilization by firategrast. B6 mice were treated with AMD3100 (5 mg/kg sc), firategrast (100 mg/kg IV), and G-CSF (250 mg/kg/d×5 days) alone or in combination. *P≤0.05, **P≤0.01, ***P≤0.001.

FIG. 26 depicts a graph showing CFC mobilization by firategrast+Gro-βt. DBA/2 mice were treated with firategrast (100 mg/kg IV) and Gro-βt (2.5 mg/kg) alone or in combination. n=5. **P≤0.01, ***P≤0.001

FIGS. 27A and 27B show HSPC mobilization and transplantation. (FIG. 27A) DBA/2 mice (n=3/cohort) were treated and blood CFC and LSK numbers were determined 0.5 h post-injection. (FIG. 27B) B6 (CD45.2+) mice were treated and blood was collected 0.5 h post-injection. Mobilized blood (pooled from n=4/cohort) were co-transplanted with 2.5×105 competitor BM (CD45.1/2+) into lethally irradiated recipients (CD45.1+, n=10/mobilizing agent). The frequency of repopulating units (RU) was calculated based on the contribution within the B-cell lymphoid and myeloid fraction 20 weeks after transplant. *P≤0.05, **P≤0.01.

FIGS. 28A-28F shows the results of the colony forming cell (CFC) assay with the integrin antagonist administered in combination with Groβt. FIG. 28A shows the number of colonies formed as a function of time while FIG. 28B and FIG. 28C show the number of CFC units at a specific time point for each mouse. These results show the results for Example Compound 35 (light gray) and Example Compound 36 (dark gray). FIGS. 28D-28F show the results of the colony forming cell (CFC) assay with only the VLA-4 inhibitor. FIG. 28D shows the number of colonies formed as a function of time while FIG. 28E and FIG. 28F show the number of CFC units at a specific time point for each mouse. These results show the results for Example Compound 35 (light gray) and Example Compound 36 (dark gray).

FIG. 29 shows the effects of blocking VLA4 with CXCR2 stimulation results in the mobilization of both LSK and CFU-C cells in DBA2/J mice. n=3 ** P<0.01 **P<0.05 2-way ANOVA with Sidak post-hoc correction.

FIG. 30A and FIG. 30B show pharmacokinetics (n=3) (FIG. 30A) and pharmacodynamics (n=4-6) (FIG. 30B) of different methods of administering the agents in DBA2/J mice.

FIGS. 31A-31C show the results of repopulation assay show that the mobilized blood results in functional HPSC from either primary (n=8-18) or secondary recipients (n=5-10) in BALB/C mice. FIG. 31A shows the study design.

FIG. 32A-FIG. 32C show the results of order of administration of the agents. FIG. 32A shows that similar results were obtained when the VLA-4 inhibitor was administered before tGro-β. Similar results were obtained for the opposite combination wherein tGro-β was administered before the VLA-4 inhibitor (FIG. 32B). *** P<0.001 The highest CFU-C was seen when the agents were administered simultaneously as shown in FIG. 32C. *** P<0.001 ** P<0.001 t-test with Holm-Sidak correlation. n=4-5 and conducted in BALB/C mice.

FIG. 33A-FIG. 33H show that compositions containing tGro-β resulted in the induction of MMP9 proteolytic activity in blood (FIG. 33A-FIG. 33C) in BALB/C mice (n=3). Myeloid cells express both CXCR2 and MMP9 (FIG. 33D-FIG. 33E) and may serve as a key cell type for HSPC mobilization in BALB/C mice. (flow cytometry n=4; real time PCR n=3-9). FIG. 33F-FIG. 33H shows that GR1+ myeloid cells are required to mobilize HSPCs in BALB/C mice. n=7-9 * P<0.05.

FIG. 34 shows the combination of a VLA-4 inhibitor and tGro-β results in similar mobilization regardless of the presence of other factors which result in poor stem cell mobilization such as diabetes.

FIG. 35A-FIG. 35B graphically depicts the effects of the triple combination of combination therapy with VLA-4 inhibitor+ CXCR4 inhibitor+ CXCR2 agonist.

FIG. 36A-FIG. 36E shows independence of VLA4 and CXCR4 blockade induced mobilization. FIG. 36A-FIG. 36B graphically depicts the CFU of vehicle treated and anti-vla-4 treated CXCR4+/+ and CXCR4−/− mice. FIG. 36C shows the CFU in wt or cxcr4 f/f mice treated with AMD3100, GroB, CWHM-823 or control. FIG. 36D-FIG. 36E show LSK/CFU-C per mL of peripheral blood.

FIG. 37A-FIG. 37C show the effects of the triple combination of combination therapy with VLA-4 inhibitor+ CXCR4 inhibitor+ CXCR2 agonist. FIG. 37A shows increased CFU-C per mL, FIG. 37B shows increased WBC per mL and FIG. 37C shows increased NE per mL of peripheral blood in triple therapy treated mice compared to the other conditions.

### DETAILED DESCRIPTION

### I. Integrin Antagonists

### (a) Background

Currently, hematopoietic stem cell transplants require the collection of the stem cells from peripheral blood. Due to the low amount of these cells in circulating peripheral blood, stimulating the stem cells can take almost a week and still the collection must be done over several days to achieve sufficient concentrations of the stem cells for transplantation. This greatly increases the cost of the transplant and results in a significant burden on the patient. Currently, cytokines, such as granulocyte-colony forming unit (G-CSF), and immunostimulants, such as plerixafor, are used to increase the amount of hematopoietic stem cells in the peripheral blood. Unfortunately, even these methods often fail to increase the concentrations to sufficient levels for transplantation. Therefore, a need remains for better methods to harvest hematopoietic stem cells for transplantation.

### (b) Summary

The present disclosure provides compounds which are VLA-4 antagonists (i.e. inhibitors), pharmaceutical compositions, methods for their manufacture, and methods for their use.

In some aspects, the present disclosure provides compounds further defined by the formula:

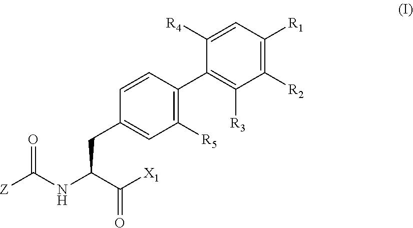

wherein:
R₁ is hydrogen, aminocarbonyl, carboxy, cyano, halo, hydroxy, —SF₅, alkyl_{(C≤8)}, substituted alkyl_{(C≤8)}, alkoxy_{(C≤8)}, substituted alkoxy_{(C≤8)}, or —Y₁—Rₐ; wherein:
   Y₁ is alkanediyl_{(C≤8)} or substituted alkanediyl_{(C≤8)}; and
   Rₐ is alkoxy_{(C≤12)}, acyloxy_{(C≤12)}, or a substituted version of either of these groups; or
—X(CH₂O)ₘ—(CH₂CH₂O)ₙ—R_{b}; wherein:
   X is a covalent bond or —O—;
   m is 0 or 1;
   n is 1, 2, 3, 4, 5, 6, 7, or 8; and
   R_{b} is hydrogen, alkyl_{(C≤8)}, or substituted alkyl_{(C≤8)}; or
—CH₂—O—(CH₂)ₙ—R_{c}; wherein:
   N is 0, 1, or 2; and
   R_{c} is a cyclic aliphatic ring of 4-6 in size having one oxygen as part of the ring;
R₂ is hydrogen, aminocarbonyl, carboxy, cyano, halo, hydroxy, —SF₅, alkyl_{(C≤8)}, substituted alkyl_{(C≤8)}, alkoxy_{(C≤8)}, substituted alkoxy_{(C≤8)}, or —Y₂—R_{c}; wherein:
   Y₂ is alkanediyl_{(C≤8)} or substituted alkanediyl_{(C≤8)}; and
   R_{c} is alkoxy_{(C≤12)}, acyloxy_{(C≤12)}, or a substituted version of either of these groups;
X₁ is hydroxy, alkoxy_{(C≤8)}, substituted alkoxy_{(C≤8)}, cycloalkoxy_{(C≤8)}, substituted cycloalkoxy_{(C≤8)}, alkenyloxy_{(C≤8)}, substituted alkenyloxy_{(C≤8)}, aryloxy_{(C≤8)}, substituted aryloxy_{(C≤8)}, aralkoxy_{(C≤8)}, substituted aralkoxy_{(C≤8)}, or a substituent convertible in vivo to hydroxy; and
R₃ and R₄ are each independently hydrogen, hydroxy, alkoxy_{(C≤8)} or substituted alkoxy_{(C≤8)};
R₅ is hydrogen, —CH(OR_{d})Rₑ, or —C(O)R_{f}, wherein:
   R_{d} is hydrogen, alkyl_{(C≤8)}, substituted alkyl_{(C≤8)}, acyl_{(C≤8)}, or substituted acyl_{(C≤8)}; and
   Rₑ and R_{f} are each independently alkyl_{(C≤8)} or substituted alkyl_{(C≤8)}; and
Z is a group of the formula:

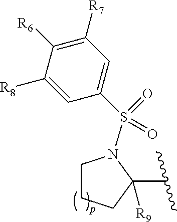

wherein:
   p is 0, 1, 2, or 3;
   R₆ is hydrogen or —C(O)X₂; wherein:
      X₂ is amino, hydroxy, alkoxy_{(C≤8)}, substituted alkoxy_{(C≤8)}, cycloalkoxy_{(C≤8)}, substituted cycloalkoxy_{(C≤8)}, alkenyloxy_{(C≤8)}, substituted alkenyloxy_{(C≤8)}, aryloxy_{(C≤8)}, substituted aryloxy_{(C≤8)}, aralkyloxy_{(C≤8)}, substituted aralkyloxy_{(C≤8)}, alkylamino_{(C≤8)}, substituted alkylamino_{(C≤8)}, dialkylamino_{(C≤8)}, substituted dialkylamino_{(C≤8)}, cycloalkyl-amino_{(C≤8)}, substituted cycloalkylamino_{(C≤8)}, alkenylamino_{(C≤8)}, substituted alkenylamino_{(C≤8)}, arylamino_{(C≤8)}, substituted arylamino_{(C≤8)}, aralkylamino_{(C≤8)}, substituted aralkylamino_{(C≤8)}, or a substituent convertible in vivo to hydroxy;
   R₇ and R₈ are each independently hydrogen, halo, haloalkyl_{(C≤8)}, or substituted haloalkyl_{(C≤8)};
   R₉ is hydrogen, alkyl_{(C≤8)}, or substituted alkyl_{(C≤8)}; or
a group of the formula:

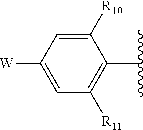

wherein:
   W is hydrogen, cyano, halo, hydroxy, or —C(O)X₃, wherein:
      X₃ is amino, hydroxy, alkoxy_{(C≤8)}, substituted alkoxy_{(C≤8)}, cycloalkoxy_{(C≤8)}, substituted cycloalkoxy_{(C≤8)}, alkenyloxy_{(C≤8)}, substituted alkenyloxy_{(C≤8)}, aryloxy_{(C≤8)}, substituted aryloxy_{(C≤8)}, aralkyloxy_{(C≤8)}, substituted aralkyloxy_{(C≤8)}, alkylamino_{(C≤8)}, substituted alkylamino_{(C≤8)}, dialkylamino_{(C≤8)}, substituted dialkylamino_{(C≤8)}, cycloalkyl-amino_{(C≤8)}, substituted cycloalkylamino_{(C≤8)}, alkenylamino_{(C≤8)}, substituted alkenylamino_{(C≤8)}, arylamino_{(C≤8)}, substituted arylamino_{(C≤8)}, aralkylamino_{(C≤8)}, substituted aralkylamino_{(C≤8)}, or a substituent convertible in vivo to hydroxy;
R₁₀ and R₁₁ are each independently hydrogen or halo. provided that if R₅ is hydrogen, then W is —C(O)X₃; and if W is —C(O)X₃ or R₆ is
—C(O)X₂, then R₉ is hydrogen; and if R₁ is hydrogen, then R₆ is not hydrogen; or a pharmaceutically acceptable salt thereof.

In some embodiments, the compounds are further defined by the formula:

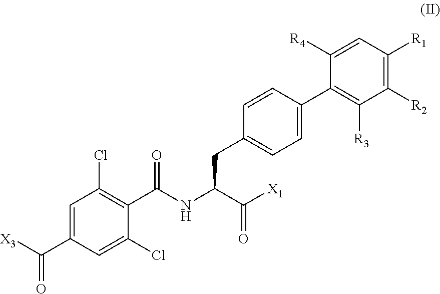

wherein:
R₁ is hydrogen, aminocarbonyl, carboxy, cyano, halo, hydroxy, —SF₅, alkyl_{(C≤8)}, substituted alkyl_{(C≤8)}, alkoxy_{(C≤8)}, substituted alkoxy_{(C≤8)}, or —Y₁—Rₐ; wherein:
   Y₁ is alkanediyl_{(C≤8)} or substituted alkanediyl_{(C≤8)}; and
   Rₐ is alkoxy_{(C≤12)}, acyloxy_{(C≤12)}, or a substituted version of either of these groups; or
—X(CH₂O)ₘ—(CH₂CH₂O)ₙ—R_{b}; wherein:
   X is a covalent bond or —O—;
   m is 0 or 1;
   n is 1, 2, 3, 4, 5, 6, 7, or 8; and
   R_{b} is hydrogen, alkyl_{(C≤8)}, or substituted alkyl_{(C≤8)}; or
—CH₂—O—(CH₂)ₙ—R_{c}; wherein:
   N is 0, 1, or 2; and
   R_{c} is a cyclic aliphatic ring of 4-6 in size having one oxygen as part of the ring;
R₂ is hydrogen, aminocarbonyl, carboxy, cyano, halo, hydroxy, —SF₅, alkyl_{(C≤8)}, substituted alkyl_{(C≤8)}, alkoxy_{(C≤8)}, substituted alkoxy_{(C≤8)}, or —Y₂—R_{c}; wherein:
   Y₂ is alkanediyl_{(C≤8)} or substituted alkanediyl_{(C≤8)}; and
   R_{c} is alkoxy_{(C≤12)}, acyloxy_{(C≤12)}, or a substituted version of either of these groups;
X₁ and X₃ are each independently hydroxy, alkoxy_{(C≤8)}, substituted alkoxy_{(C≤8)}, cycloalkoxy_{(C≤8)}, substituted cycloalkoxy_{(C≤8)}, alkenyloxy_{(C≤8)}, substituted alkenyloxy_{(C≤8)}, aryloxy_{(C≤8)}, substituted aryloxy_{(C≤8)}, aralkoxy_{(C≤8)}, substituted aralkoxy_{(C≤8)}, or a substituent convertible in vivo to hydroxy; and
R₃ and R₄ are each independently alkoxy_{(C≤8)} or substituted alkoxy_{(C≤8)};
provided that R₁ and R₂ are not both hydrogen;
   or a pharmaceutically acceptable salt thereof.

In some embodiments, the compounds are further defined as:

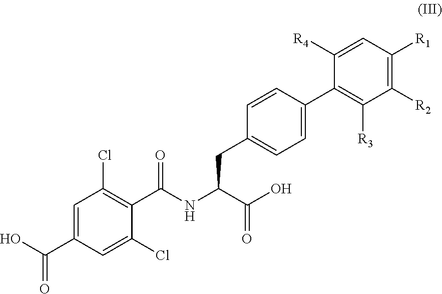

wherein:
R₁ is hydrogen, aminocarbonyl, carboxy, cyano, halo, hydroxy, —SF₅, alkyl_{(C≤8)}, substituted alkyl_{(C≤8)}, alkoxy_{(C≤8)}, substituted alkoxy_{(C≤8)}, or —Y₁—Rₐ; wherein:
   Y₁ is alkanediyl_{(C≤8)} or substituted alkanediyl_{(C≤8)}; and
   Rₐ is alkoxy_{(C≤12)}, acyloxy_{(C≤12)}, or a substituted version of either of these groups; or
—X(CH₂O)ₘ—(CH₂CH₂O)ₙ—R_{b}; wherein:
   X is a covalent bond or —O—;
   m is 0 or 1;
   n is 1, 2, 3, 4, 5, 6, 7, or 8; and
   R_{b} is hydrogen, alkyl_{(C≤8)}, or substituted alkyl_{(C≤8)}; or
—CH₂—O—(CH₂)ₙ—R_{c}; wherein:
   N is 0, 1, or 2; and
   R_{c} is a cyclic aliphatic ring of 4-6 in size having one oxygen as part of the ring;
R₂ is hydrogen, aminocarbonyl, carboxy, cyano, halo, hydroxy, —SF₅, alkyl_{(C≤8)}, substituted alkyl_{(C≤8)}, alkoxy_{(C≤8)}, substituted alkoxy_{(C≤8)}, or —Y₂—R_{c}; wherein:
   Y₂ is alkanediyl_{(C≤8)} or substituted alkanediyl_{(C≤8)}; and
   R_{c} is alkoxy_{(C≤12)}, acyloxy_{(C≤12)}, or a substituted version of either of these groups; and
R₃ and R₄ are each independently alkoxy_{(C≤8)} or substituted alkoxy_{(C≤8)};
provided that R₁ and R₂ are not both hydrogen;
   or a pharmaceutically acceptable salt thereof.

In some embodiments, the compounds are further defined as:

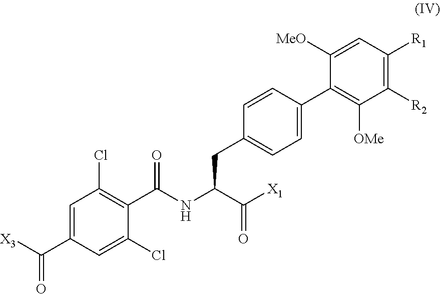

wherein:
R₁ is aminocarbonyl, carboxy, cyano, halo, hydroxy, —SF₅, alkyl_{(C≤8)}, substituted alkyl_{(C≤8)}, alkoxy_{(C≤8)}, substituted alkoxy_{(C≤8)}, or —Y₁—Rₐ; wherein:
   Y₁ is alkanediyl_{(C≤8)} or substituted alkanediyl_{(C≤8)}; and
   Rₐ is alkoxy_{(C≤12)}, acyloxy_{(C≤12)}, or a substituted version of either of these groups; or
—X(CH₂O)ₘ—(CH₂CH₂O)ₙ—R_{b}; wherein:
   X is a covalent bond or —O—;
   m is 0 or 1;
   n is 1, 2, 3, 4, 5, 6, 7, or 8; and
   R_{b} is hydrogen, alkyl_{(C≤8)}, or substituted alkyl_{(C≤8)}; or
—CH₂—O—(CH₂)ₙ—R_{c}; wherein:
   N is 0, 1, or 2; and
   R_{c} is a cyclic aliphatic ring of 4-6 in size having one oxygen as part of the ring;
R₂ is hydrogen, aminocarbonyl, carboxy, cyano, halo, hydroxy, —SF₅, alkyl_{(C≤8)}, substituted alkyl_{(C≤8)}, alkoxy_{(C≤8)}, substituted alkoxy_{(C≤8)}, or —Y₂—R_{c}; wherein:
   Y₂ is alkanediyl_{(C≤8)} or substituted alkanediyl_{(C≤8)}; and
   R_{c} is alkoxy_{(C≤12)}, acyloxy_{(C≤12)}, or a substituted version of either of these groups;
X₁ and X₃ are each independently hydroxy, alkoxy_{(C≤8)}, substituted alkoxy_{(C≤8)}, cycloalkoxy_{(C≤8)}, substituted cycloalkoxy_{(C≤8)}, alkenyloxy_{(C≤8)}, substituted alkenyloxy_{(C≤8)}, aryloxy_{(C≤8)}, substituted aryloxy_{(C≤8)}, aralkoxy_{(C≤8)}, substituted aralkoxy_{(C≤8)}, or a group convertible in vivo to hydroxy;
   or a pharmaceutically acceptable salt thereof.

In some embodiments, the compounds are further defined as:

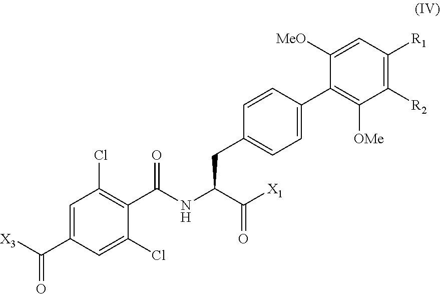

wherein:
R₁ is hydrogen, aminocarbonyl, carboxy, cyano, halo, hydroxy, —SF₅, alkyl_{(C≤8)}, substituted alkyl_{(C≤8)}, alkoxy_{(C≤8)}, substituted alkoxy_{(C≤8)}, or —Y₁—Rₐ; wherein:
   Y₁ is alkanediyl_{(C≤8)} or substituted alkanediyl_{(C≤8)}; and
   Rₐ is alkoxy_{(C≤12)}, acyloxy_{(C≤12)}, or a substituted version of either of these groups; or
—X(CH₂O)ₘ—(CH₂CH₂O)ₙ—R_{b}; wherein:
   X is a covalent bond or —O—;
   m is 0 or 1;
   n is 1, 2, 3, 4, 5, 6, 7, or 8; and
   R_{b} is hydrogen, alkyl_{(C≤8)}, or substituted alkyl_{(C≤8)}; or
—CH₂—O—(CH₂)ₙ—R_{c}; wherein:
   N is 0, 1, or 2; and
   R_{c} is a cyclic aliphatic ring of 4-6 in size having one oxygen as part of the ring;
R₂ is aminocarbonyl, carboxy, cyano, halo, hydroxy, —SF₅, alkyl_{(C≤8)}, substituted alkyl_{(C≤8)}, alkoxy_{(C≤8)}, substituted alkoxy_{(C≤8)}, or —Y₂—R_{c}; wherein:
   Y₂ is alkanediyl_{(C≤8)} or substituted alkanediyl_{(C≤8)}; and
   R_{c} is alkoxy_{(C≤12)}, acyloxy_{(C≤12)}, or a substituted version of either of these groups;
X₁ and X₃ are each independently hydroxy, alkoxy_{(C≤8)}, substituted alkoxy_{(C≤8)}, cycloalkoxy_{(C≤8)}, substituted cycloalkoxy_{(C≤8)}, alkenyloxy_{(C≤8)}, substituted alkenyloxy_{(C≤8)}, aryloxy_{(C≤8)}, substituted aryloxy_{(C≤8)}, aralkoxy_{(C≤8)}, substituted aralkoxy_{(C≤8)}, or a group convertible in vivo to hydroxy; or a pharmaceutically acceptable salt thereof.

In some embodiments, the compounds are further defined as:

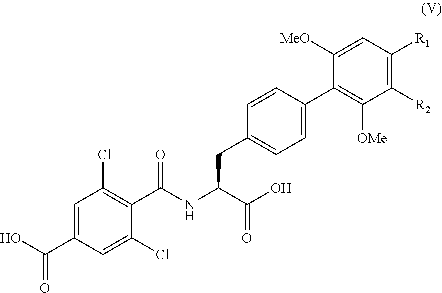

wherein:
R₁ is hydrogen, aminocarbonyl, carboxy, cyano, halo, hydroxy, —SF₅, alkyl_{(C≤8)}, substituted alkyl_{(C≤8)}, alkoxy_{(C≤8)}, substituted alkoxy_{(C≤8)}, or —Y₁—Rₐ; wherein:
   Y₁ is alkanediyl_{(C≤8)} or substituted alkanediyl_{(C≤8)}; and
   Rₐ is alkoxy_{(C≤12)}, acyloxy_{(C≤12)}, or a substituted version of either of these groups; or
—X(CH₂O)ₘ—(CH₂CH₂O)ₙ—R_{b}; wherein:
   X is a covalent bond or —O—;
   m is 0 or 1;
   n is 1, 2, 3, 4, 5, 6, 7, or 8; and
   R_{b} is hydrogen, alkyl_{(C≤8)}, or substituted alkyl_{(C≤8)}; or
—CH₂—O—(CH₂)ₙ—R_{c}; wherein:
   N is 0, 1, or 2; and
   R_{c} is a cyclic aliphatic ring of 4-6 in size having one oxygen as part of the ring; or
—CH₂—O—(CH₂)ₙ—R_{c}; wherein:
   N is 0, 1, or 2; and
   R_{c} is a cyclic aliphatic ring of 4-6 in size having one oxygen as part of the ring;
R₂ is hydrogen, aminocarbonyl, carboxy, cyano, halo, hydroxy, —SF₅, alkyl_{(C≤8)}, substituted alkyl_{(C≤8)}, alkoxy_{(C≤8)}, substituted alkoxy_{(C≤8)}, or —Y₂—R_{c}; wherein:
   Y₂ is alkanediyl_{(C≤8)} or substituted alkanediyl_{(C≤8)}; and
   R_{c} is alkoxy_{(C≤12)}, acyloxy_{(C≤12)}, or a substituted version of either of these groups;
provided that R₁ and R₂ are not both hydrogen;
   or a pharmaceutically acceptable salt thereof.

In some embodiments, the compounds are further defined as:

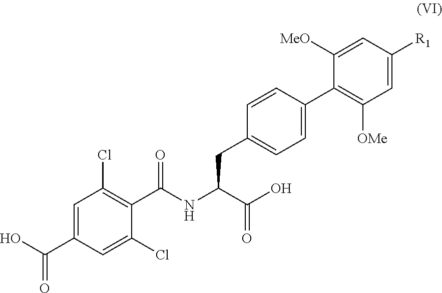

wherein:
R₁ is alkyl_{(C≤8)}, substituted alkyl_{(C≤8)}, alkoxy_{(C≤8)}, substituted alkoxy_{(C≤8)}, or —Y₁—Rₐ; wherein:
   Y₁ is alkanediyl_{(C≤8)} or substituted alkanediyl_{(C≤8)}; and
   Rₐ is alkoxy_{(C≤12)}, acyloxy_{(C≤12)}, or a substituted version of either of these groups; or
—X(CH₂O)ₘ—(CH₂CH₂O)ₙ—R_{b}; wherein:
   X is a covalent bond or —O—;
   m is 0 or 1;
   n is 1, 2, 3, 4, 5, 6, 7, or 8; and
   R_{b} is hydrogen, alkyl_{(C≤8)}, or substituted alkyl_{(C≤8)}; or
—CH₂—O—(CH₂)ₙ—R_{c}; wherein:
   N is 0, 1, or 2; and
   R_{c} is a cyclic aliphatic ring of 4-6 in size having one oxygen as part of the ring;
      or a pharmaceutically acceptable salt thereof.

In other embodiments, the compounds are further defined as:

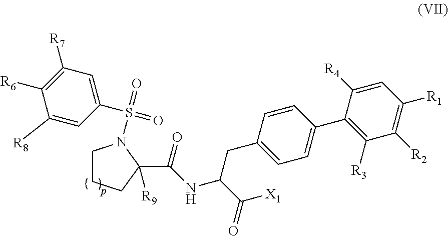

wherein:
R₁ is hydrogen, aminocarbonyl, carboxy, cyano, halo, hydroxy, —SF₅, alkyl_{(C≤8)}, substituted alkyl_{(C≤8)}, alkoxy_{(C≤8)}, substituted alkoxy_{(C≤8)}, or —Y₁—Rₐ, wherein:
   Y₁ is alkanediyl_{(C≤8)} or substituted alkanediyl_{(C≤8)}; and
   Rₐ is alkoxy_{(C≤12)}, acyloxy_{(C≤12)}, or a substituted version of either group; or
—X(CH₂O)ₘ—(CH₂CH₂O)ₙ—R_{b}, wherein:
   X is a covalent bond or —O—;
   m is 0 or 1;
   n is 1, 2, 3, 4, 5, 6, 7, or 8; and
   R_{b} is hydrogen, alkyl_{(C≤8)}, or substituted alkyl_{(C≤8)}; or
—CH₂—O—(CH₂)ₙ—R_{c}; wherein:
   N is 0, 1, or 2; and
   R_{c} is a cyclic aliphatic ring of 4-6 in size having one oxygen as part of the ring;
R₂ is hydrogen, aminocarbonyl, carboxy, cyano, halo, hydroxy, —SF₅, alkyl_{(C≤8)}, substituted alkyl_{(C≤8)}, alkoxy_{(C≤8)}, substituted alkoxy_{(C≤8)}, or —Y₂—R_{c}, wherein:
   Y₂ is alkanediyl_{(C≤8)} or substituted alkanediyl_{(C≤8)}; and
   R_{c} is alkoxy_{(C≤12)}, acyloxy_{(C≤12)}, or a substituted version of either group; or
R₃ and R₄ are each independently alkoxy_{(C≤8)} or substituted alkoxy_{(C≤8)};
R₇ and R₈ are each independently hydrogen, halo, haloalkyl_{(C≤8)}, or substituted haloalkyl_{(C≤8)};
R₆ is hydrogen or —C(O)X₂;
R₉ is hydrogen, alkyl_{(C≤6)}, or substituted alkyl_{(C≤6)};
p is 0, 1, 2, or 3; and
X₁ and X₂ are each independently hydroxy, alkoxy_{(C≤8)}, substituted alkoxy_{(C≤8)}, cycloalkoxy_{(C≤8)}, substituted cycloalkoxy_{(C≤8)}, alkenyloxy_{(C≤8)}, substituted alkenyloxy_{(C≤8)}, aryloxy_{(C≤8)}, substituted aryloxy_{(C≤8)}, aralkyloxy_{(C≤8)}, substituted aralkyloxy_{(C≤8)}, or a substituent convertible in vivo to hydroxy;
provided that R₁ and R₇ are not both hydrogen;
   or a pharmaceutically acceptable salt thereof.

In some embodiments, the compounds are further defined as:

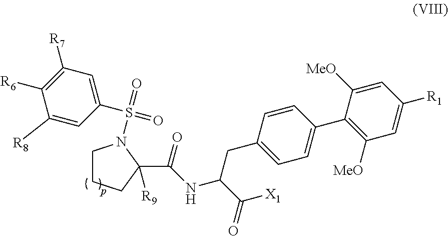

wherein:
R₁ is hydrogen, aminocarbonyl, carboxy, cyano, halo, hydroxy, —SF₅, alkyl_{(C≤8)}, substituted alkyl_{(C≤8)}, alkoxy_{(C≤8)}, substituted alkoxy_{(C≤8)}, or —Y₁—Rₐ, wherein:
   Y₁ is alkanediyl_{(C≤8)} or substituted alkanediyl_{(C≤8)}; and
   Rₐ is alkoxy_{(C≤12)}, acyloxy_{(C≤12)}, or a substituted version of either group; or
—X(CH₂O)ₘ—(CH₂CH₂O)ₙ—R_{b}, wherein:
   X is a covalent bond or —O—;
   m is 0 or 1;
   n is 1, 2, 3, 4, 5, 6, 7, or 8; and
   R_{b} is hydrogen, alkyl_{(C≤8)}, or substituted alkyl_{(C≤8)}; or
—CH₂—O—(CH₂)ₙ—R_{c}; wherein:
   N is 0, 1, or 2; and
   R_{c} is a cyclic aliphatic ring of 4-6 in size having one oxygen as part of the ring;
R₇ and R₈ are each independently hydrogen, halo, haloalkyl_{(C≤8)}, or substituted haloalkyl_{(C≤8)};
R₆ is hydrogen or —C(O)X₂;
R₉ is hydrogen, alkyl_{(C≤6)}, or substituted alkyl_{(C≤6)};
p is 0, 1, 2, or 3; and
X₁ and X₂ are each independently hydroxy, alkoxy_{(C≤8)}, substituted alkoxy_{(C≤8)}, cycloalkoxy_{(C≤8)}, substituted cycloalkoxy_{(C≤8)}, alkenyloxy_{(C≤8)}, substituted alkenyloxy_{(C≤8)}, aryloxy_{(C≤8)}, substituted aryloxy_{(C≤8)}, aralkyloxy_{(C≤8)}, substituted aralkyloxy_{(C≤8)}, or a substituent convertible in vivo to hydroxy;
provided that R₁ and R₇ are not both hydrogen;
   or a pharmaceutically acceptable salt thereof.

In some embodiments, the compounds are further defined as:

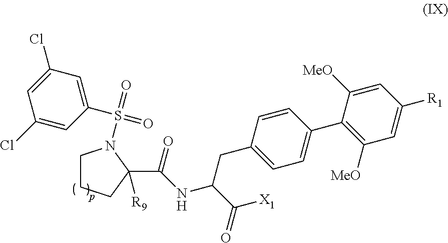

wherein:
R₁ is aminocarbonyl, carboxy, cyano, halo, hydroxy, —SF₅, alkyl_{(C≤8)}, substituted alkyl_{(C≤8)}, alkoxy_{(C≤8)}, substituted alkoxy_{(C≤8)}, or —Y₁—Rₐ, wherein:
   Y₁ is alkanediyl_{(C≤8)} or substituted alkanediyl_{(C≤8)}; and
   Rₐ is alkoxy_{(C≤12)}, acyloxy_{(C≤12)}, or a substituted version of either group; or
—X(CH₂O)ₘ—(CH₂CH₂O)ₙ—R_{b}, wherein:
   X is a covalent bond or —O—;
   m is 0 or 1;
   n is 1, 2, 3, 4, 5, 6, 7, or 8; and
   R_{b} is hydrogen, alkyl_{(C≤8)}, or substituted alkyl_{(C≤8)}; or
—CH₂—O—(CH₂)ₙ—R_{c}; wherein:
   N is 0, 1, or 2; and
   R_{c} is a cyclic aliphatic ring of 4-6 in size having one oxygen as part of the ring;
R₉ is hydrogen, alkyl_{(C≤6)}, or substituted alkyl_{(C≤6)};
p is 0, 1, 2, or 3; and
X₁ is hydroxy, alkoxy_{(C≤8)}, substituted alkoxy_{(C≤8)}, cycloalkoxy_{(C≤8)}, substituted cycloalkoxy_{(C≤8)}, alkenyloxy_{(C≤8)}, substituted alkenyloxy_{(C≤8)}, aryloxy_{(C≤8)}, substituted aryloxy_{(C≤8)}, aralkyloxy_{(C≤8)}, substituted aralkyloxy_{(C≤8)}, or a substituent convertible in vivo to hydroxy;
   or a pharmaceutically acceptable salt thereof.

In some embodiments, the compounds are further defined as:

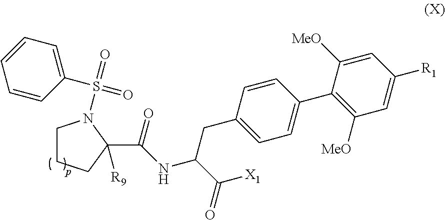

wherein:
R₁ is aminocarbonyl, carboxy, cyano, halo, hydroxy, —SF₅, alkyl_{(C≤8)}, substituted alkyl_{(C≤8)}, alkoxy_{(C≤8)}, substituted alkoxy_{(C≤8)}, or —Y₁—Rₐ, wherein:
   Y₁ is alkanediyl_{(C≤8)} or substituted alkanediyl_{(C≤8)}; and
   Rₐ is alkoxy_{(C≤12)}, acyloxy_{(C≤12)}, or a substituted version of either group; or
—X(CH₂O)ₘ—(CH₂CH₂O)ₙ—R_{b}, wherein:
   X is a covalent bond or —O—;
   m is 0 or 1;
   n is 1, 2, 3, 4, 5, 6, 7, or 8; and
   R_{b} is hydrogen, alkyl_{(C≤8)}, or substituted alkyl_{(C≤8)}; or
—CH₂—O—(CH₂)ₙ—R_{c}; wherein:
   N is 0, 1, or 2; and
   R_{c} is a cyclic aliphatic ring of 4-6 in size having one oxygen as part of the ring;
R₉ is hydrogen, alkyl_{(C≤6)}, or substituted alkyl_{(C≤6)};
p is 0, 1, 2, or 3; and
X₁ is hydroxy, alkoxy_{(C≤8)}, substituted alkoxy_{(C≤8)}, cycloalkoxy_{(C≤8)}, substituted cycloalkoxy_{(C≤8)}, alkenyloxy_{(C≤8)}, substituted alkenyloxy_{(C≤8)}, aryloxy_{(C≤8)}, substituted aryloxy_{(C≤8)}, aralkyloxy_{(C≤8)}, substituted aralkyloxy_{(C≤8)}, or a substituent convertible in vivo to hydroxy;
   or a pharmaceutically acceptable salt thereof.

In some embodiments, the compounds are further defined as:

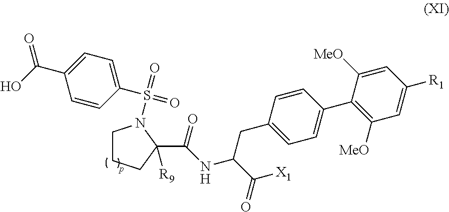

wherein:
R₁ is aminocarbonyl, carboxy, cyano, halo, hydroxy, —SF₅, alkyl_{(C≤8)}, substituted alkyl_{(C≤8)}, alkoxy_{(C≤8)}, substituted alkoxy_{(C≤8)}, or —Y₁—Rₐ, wherein:
   Y₁ is alkanediyl_{(C≤8)} or substituted alkanediyl_{(C≤8)}; and
   Rₐ is alkoxy_{(C≤12)}, acyloxy_{(C≤12)}, or a substituted version of either group; or
—X(CH₂O)ₘ—(CH₂CH₂O)ₙ—R_{b}, wherein:
   X is a covalent bond or —O—;
   m is 0 or 1;
   n is 1, 2, 3, 4, 5, 6, 7, or 8; and
   R_{b} is hydrogen, alkyl_{(C≤8)}, or substituted alkyl_{(C≤8)}; or
—CH₂—O—(CH₂)ₙ—R_{c}; wherein:
   N is 0, 1, or 2; and
   R_{c} is a cyclic aliphatic ring of 4-6 in size having one oxygen as part of the ring;
R₉ is hydrogen, alkyl_{(C≤6)}, or substituted alkyl_{(C≤6)};
p is 0, 1, 2, or 3; and
X₁ is hydroxy, alkoxy_{(C≤8)}, substituted alkoxy_{(C≤8)}, cycloalkoxy_{(C≤8)}, substituted cycloalkoxy_{(C≤8)}, alkenyloxy_{(C≤8)}, substituted alkenyloxy_{(C≤8)}, aryloxy_{(C≤8)}, substituted aryloxy_{(C≤8)}, aralkyloxy_{(C≤8)}, substituted aralkyloxy_{(C≤8)}, or a substituent convertible in vivo to hydroxy;
   or a pharmaceutically acceptable salt thereof.

In other embodiments, the compounds are further defined as:

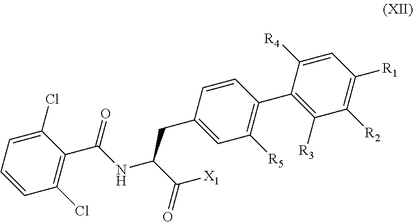

wherein:
R₁ is aminocarbonyl, carboxy, cyano, halo, hydroxy, —SF₅, alkyl_{(C≤8)}, substituted alkyl_{(C≤8)}, alkoxy_{(C≤8)}, substituted alkoxy_{(C≤8)}, or —Y₁—Rₐ, wherein:
   Y₁ is alkanediyl_{(C≤8)} or substituted alkanediyl_{(C≤8)}; and
   Rₐ is alkoxy_{(C≤12)}, acyloxy_{(C≤12)}, or a substituted version of either group; or
—X(CH₂O)ₘ—(CH₂CH₂O)ₙ—R_{b}, wherein:
   X is a covalent bond or —O—;
   m is 0 or 1;
   n is 1, 2, 3, 4, 5, 6, 7, or 8; and
   R_{b} is hydrogen, alkyl_{(C≤8)}, or substituted alkyl_{(C≤8)}; or
—CH₂—O—(CH₂)ₙ—R_{c}; wherein:
   N is 0, 1, or 2; and
   R_{c} is a cyclic aliphatic ring of 4-6 in size having one oxygen as part of the ring;
R₂ is hydrogen, aminocarbonyl, carboxy, cyano, halo, hydroxy, —SF₅, alkyl_{(C≤8)}, substituted alkyl_{(C≤8)}, alkoxy_{(C≤8)}, substituted alkoxy_{(C≤8)}, or —Y₂—R_{c}, wherein:
   Y₂ is alkanediyl_{(C≤8)} or substituted alkanediyl_{(C≤8)}; and
   R_{c} is alkoxy_{(C≤12)}, alkoxy_{(C≤12)}, or a substituted version of either group;
R₃ and R₄ are each independently alkoxy_{(C≤8)} or substituted alkoxy_{(C≤8)};
R₅ is hydrogen, —CH(OR_{d})Rₑ, or —C(O)R_{f}, wherein:
   R_{d} is hydrogen, alkyl_{(C≤8)}, substituted alkyl_{(C≤8)}, acyl_{(C≤8)}, or substituted acyl_{(C≤8)}; and
   Rₑ and R_{f} are each independently alkyl_{(C≤8)} or substituted alkyl_{(C≤8)};
X₁ is hydroxy, alkoxy_{(C≤8)}, substituted alkoxy_{(C≤8)}, cycloalkoxy_{(C≤8)}, substituted cycloalkoxy_{(C≤8)}, alkenyloxy_{(C≤8)}, substituted alkenyloxy_{(C≤8)}, aryloxy_{(C≤8)}, substituted aryloxy_{(C≤8)}, aralkyloxy_{(C≤8)}, substituted aralkyloxy_{(C≤8)}, or a substituent convertible in vivo to hydroxy;
   or a pharmaceutically acceptable salt thereof.

In some embodiments, the compounds are further defined as:

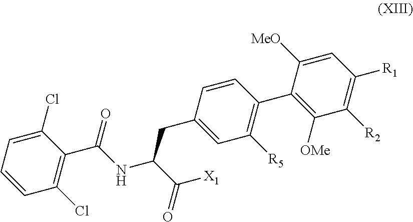

wherein:
R₁ is aminocarbonyl, carboxy, cyano, halo, hydroxy, —SF₅, alkyl_{(C≤8)}, substituted alkyl_{(C≤8)}, alkoxy_{(C≤8)}, substituted alkoxy_{(C≤8)}, or —Y₁—Rₐ, wherein:
   Y₁ is alkanediyl_{(C≤8)} or substituted alkanediyl_{(C≤8)}; and
   Rₐ is alkoxy_{(C≤12)}, acyloxy_{(C≤12)}, or a substituted version of either group; or
—X(CH₂O)ₘ—(CH₂CH₂O)ₙ—R_{b}, wherein:
   X is a covalent bond or —O—;
   m is 0 or 1;
   n is 1, 2, 3, 4, 5, 6, 7, or 8; and
   R_{b} is hydrogen, alkyl_{(C≤8)}, or substituted alkyl_{(C≤8)}; or
—CH₂—O—(CH₂)ₙ—R_{c}; wherein:
   N is 0, 1, or 2; and
   R_{c} is a cyclic aliphatic ring of 4-6 in size having one oxygen as part of the ring;
R₂ is hydrogen, aminocarbonyl, carboxy, cyano, halo, hydroxy, —SF₅, alkyl_{(C≤8)}, substituted alkyl_{(C≤8)}, alkoxy_{(C≤8)}, substituted alkoxy_{(C≤8)}, or —Y₂—R_{c}, wherein:
   Y₂ is alkanediyl_{(C≤8)} or substituted alkanediyl_{(C≤8)}; and
   R_{c} is alkoxy_{(C≤12)}, alkoxy_{(C≤12)}, or a substituted version of either group;
R₅ is hydrogen, —CH(OR_{d})Rₑ, or —C(O)R_{f}, wherein:
   R_{d} is hydrogen, alkyl_{(C≤8)}, substituted alkyl_{(C≤8)}, acyl_{(C≤8)}, or substituted acyl_{(C≤8)}; and
   Rₑ and R_{f} are each independently alkyl_{(C≤8)} or substituted alkyl_{(C≤8)};
X₁ is hydroxy, alkoxy_{(C≤8)}, substituted alkoxy_{(C≤8)}, cycloalkoxy_{(C≤8)}, substituted cycloalkoxy_{(C≤8)}, alkenyloxy_{(C≤8)}, substituted alkenyloxy_{(C≤8)}, aryloxy_{(C≤8)}, substituted aryloxy_{(C≤8)}, aralkyloxy_{(C≤8)}, substituted aralkyloxy_{(C≤8)}, or a substituent convertible in vivo to hydroxy;
   or a pharmaceutically acceptable salt thereof.

In some embodiments, the compounds are further defined as:

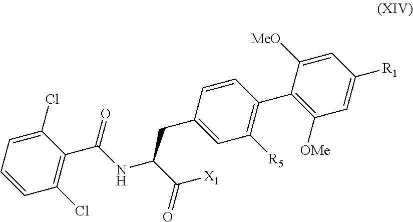

wherein:
R₁ is aminocarbonyl, carboxy, cyano, halo, hydroxy, —SF₅, alkyl_{(C≤8)}, substituted alkyl_{(C≤8)}, alkoxy_{(C≤8)}, substituted alkoxy_{(C≤8)}, or —Y₁—Rₐ, wherein:
   Y₁ is alkanediyl_{(C≤8)} or substituted alkanediyl_{(C≤8)}; and
   Rₐ is alkoxy_{(C≤12)}, acyloxy_{(C≤12)}, or a substituted version of either group; or
—X(CH₂O)ₘ—(CH₂CH₂O)ₙ—R_{b}, wherein:
   X is a covalent bond or —O—;
   m is 0 or 1;
   n is 1, 2, 3, 4, 5, 6, 7, or 8; and
   R_{b} is hydrogen, alkyl_{(C≤8)}, or substituted alkyl_{(C≤8)}; or
—CH₂—O—(CH₂)ₙ—R_{c}; wherein:
   N is 0, 1, or 2; and
   R_{c} is a cyclic aliphatic ring of 4-6 in size having one oxygen as part of the ring;
R₅ is hydrogen, —CH(OR_{d})Rₑ, or —C(O)R_{f}, wherein:
   R_{d} is hydrogen, alkyl_{(C≤8)}, substituted alkyl_{(C≤8)}, acyl_{(C≤8)}, or substituted acyl_{(C≤8)}; and
   Rₑ and R_{f} are each independently alkyl_{(C≤8)} or substituted alkyl_{(C≤8)};
X₁ is hydroxy, alkoxy_{(C≤8)}, substituted alkoxy_{(C≤8)}, cycloalkoxy_{(C≤8)}, substituted cycloalkoxy_{(C≤8)}, alkenyloxy_{(C≤8)}, substituted alkenyloxy_{(C≤8)}, aryloxy_{(C≤8)}, substituted aryloxy_{(C≤8)}, aralkyloxy_{(C≤8)}, substituted aralkyloxy_{(C≤8)}, or a substituent convertible in vivo to hydroxy;
   or a pharmaceutically acceptable salt thereof.

In some embodiments, Z is:

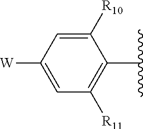

wherein:
W is hydrogen, cyano, halo, hydroxy, or —C(O)X₃, wherein:
   X₃ is amino, hydroxy, alkoxy_{(C≤8)}, substituted alkoxy_{(C≤8)}, cycloalkoxy_{(C≤8)}, substituted cycloalkoxy_{(C≤8)}, alkenyloxy_{(C≤8)}, substituted alkenyloxy_{(C≤8)}, aryloxy_{(C≤8)}, substituted aryloxy_{(C≤8)}, aralkyloxy_{(C≤8)}, substituted aralkyloxy_{(C≤8)}, alkylamino_{(C≤8)}, substituted alkylamino_{(C≤8)}, dialkylamino_{(C≤8)}, substituted dialkylamino_{(C≤8)}, cycloalkylamino_{(C≤8)}, substituted cycloalkylamino_{(C≤8)}, alkenylamino_{(C≤8)}, substituted alkenyl-amino_{(C≤8)}, arylamino_{(C≤8)}, substituted arylamino_{(C≤8)}, aralkylamino_{(C≤8)}, substituted aralkylamino_{(C≤8)}, or a substituent convertible in vivo to hydroxy; and
R₁₀ and R₁₁ are each independently hydrogen or halo.

In some embodiments, Z is:

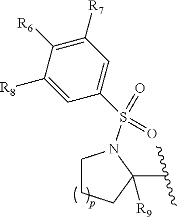

wherein:
p is 0, 1, 2, or 3;
R₆ is hydrogen or —C(O)X₂; wherein:
   X₂ is amino, hydroxy, alkoxy_{(C≤8)}, substituted alkoxy_{(C≤8)}, cycloalkoxy_{(C≤8)}, substituted cycloalkoxy_{(C≤8)}, alkenyloxy_{(C≤8)}, substituted alkenyloxy_{(C≤8)}, aryloxy_{(C≤8)}, substituted aryloxy_{(C≤8)}, aralkyloxy_{(C≤8)}, substituted aralkyloxy_{(C≤8)}, alkylamino_{(C≤8)}, substituted alkylamino_{(C≤8)}, dialkylamino_{(C≤8)}, substituted dialkylamino_{(C≤8)}, cycloalkylamino_{(C≤8)}, substituted cycloalkyl-amino_{(C≤8)}, alkenylamino_{(C≤8)}, substituted alkenylamino_{(C≤8)}, arylamino_{(C≤8)}, substituted arylamino_{(C≤8)}, aralkylamino_{(C≤8)}, substituted aralkylamino_{(C≤8)}, or a substituent convertible in vivo to hydroxy;
R₇ and R₈ are each independently hydrogen, halo, haloalkyl_{(C≤8)}, or substituted haloalkyl_{(C≤8)}; and
R₉ is hydrogen, alkyl_{(C≤8)}, or substituted alkyl_{(C≤8)}.

In some embodiments, R₁ is alkyl_{(C≤8)} or substituted alkyl_{(C≤8)} such as hydroxyalkyl_{(C≤8)} or haloalkyl_{(C≤8)}. In some embodiments, R₁ is an unbranched group. In some embodiments, R₁ is —CH₃, —CH₂OH, —CH(CH₃)OH, —C(CH₃)₂OH, —CH₂F, —CHF₂, —CF₃, —CH₂OCH₃, or —CH₂OCH₂CH₃. In some embodiments, R₁ is alkoxy_{(C≤8)} or substituted alkoxy_{(C≤8)}, such as methoxy or ethoxy. In other embodiments, R₁ is aminocarbonyl or carboxy. In other embodiments, R1 is —CH₂—O—(CH2)ₙ—R_{c}; wherein N is 0, 1, or 2; and R_{c} is a cyclic aliphatic ring of 4-6 in size having one oxygen as part of the ring

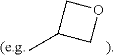

In other embodiments, R₁ is hydroxy, alkyl_{(C≤8)}, substituted alkyl_{(C≤8)}, alkoxy_{(C≤8)}, substituted alkoxy_{(C≤8)}, or —Y₁—Rₐ, wherein:

Y₁ is alkanediyl_{(C≤8)} or substituted alkanediyl_{(C≤8)}; and

Rₐ is alkoxy_{(C≤12)}, acyloxy_{(C≤12)}, or a substituted version of either group; or

—X(CH₂O)ₘ—(CH₂CH₂O)ₙ—R_{b}, wherein:

X is a covalent bond or —O—;

m is 0 or 1;

n is 1, 2, 3, 4, 5, 6, 7, or 8; and

R_{b} is hydrogen, alkyl_{(C≤8)}, or substituted alkyl_{(C≤8)}.

In other embodiments, R₁ is —Y₁—Rₐ, wherein: Y₁ is alkanediyl_{(C≤8)} or substituted alkanediyl_{(C≤8)} and Rₐ is alkoxy_{(C≤12)}, acyloxy_{(C≤12)}, or a substituted version of either group. In some embodiments, Y₁ is alkanediyl_{(C≤8)}, such as —CH₂—. In some embodiments, Rₐ is alkoxy_{(C≤12)}, such as ethoxy or hexyloxy. In some embodiments, Rₐ is acyloxy_{(C≤12)}, such as hexanoate. In other embodiments, R₁ is —X(CH₂O)ₘ—(CH₂CH₂O)ₙ—R_{b}, wherein:

X is a covalent bond or —O—;

m is 0 or 1;

n is 1, 2, 3, 4, 5, 6, 7, or 8; and

R_{b} is hydrogen, alkyl_{(C≤8)}, or substituted alkyl_{(C≤8)}.

In some embodiments, X is a covalent bond. In some embodiments, m is 1. In some embodiments, n is 1, 2, or 3. In some embodiments, n is 1. In some embodiments, R_{b} is alkyl_{(C≤8)}, such as methyl. In some embodiments, R₂ is hydrogen. In some embodiments, R₃ is alkoxy_{(C≤6)}, such as methoxy. In some embodiments, R₄ is alkoxy_{(C≤6)}, such as methoxy. In some embodiments, X₁ is hydroxy. In other embodiments, X₁ is a substituent convertible in vivo to hydroxy. In some embodiments, X₂ is hydroxy. In other embodiments, X₂ is a substituent convertible in vivo to hydroxy. In some embodiments, R₇ is hydrogen. In other embodiments, R₇ is halo, such as chloro. In some embodiments, R₈ is hydrogen. In other embodiments, R₈ is halo, such as chloro. In some embodiments, R₆ is hydrogen. In some embodiments, R₉ is hydrogen. In other embodiments, R₉ is alkyl_{(C≤8)}. In some embodiments, R₉ is alkyl_{(C≤4)}, such as methyl.

In some embodiments, p is 0, 1, or 2. In some embodiments, p is 1 or 2. In some embodiments, R₁₀ is halo, such as chloro. In some embodiments, R₁₁ is halo, such as chloro. In some embodiments, W is hydrogen. In other embodiments, W is —C(O)X₃. In other embodiments, X₃ is hydroxy. In other embodiments, X₃ is a substituent convertible in vivo to hydroxy.

In some embodiments, the compound is further defined as:

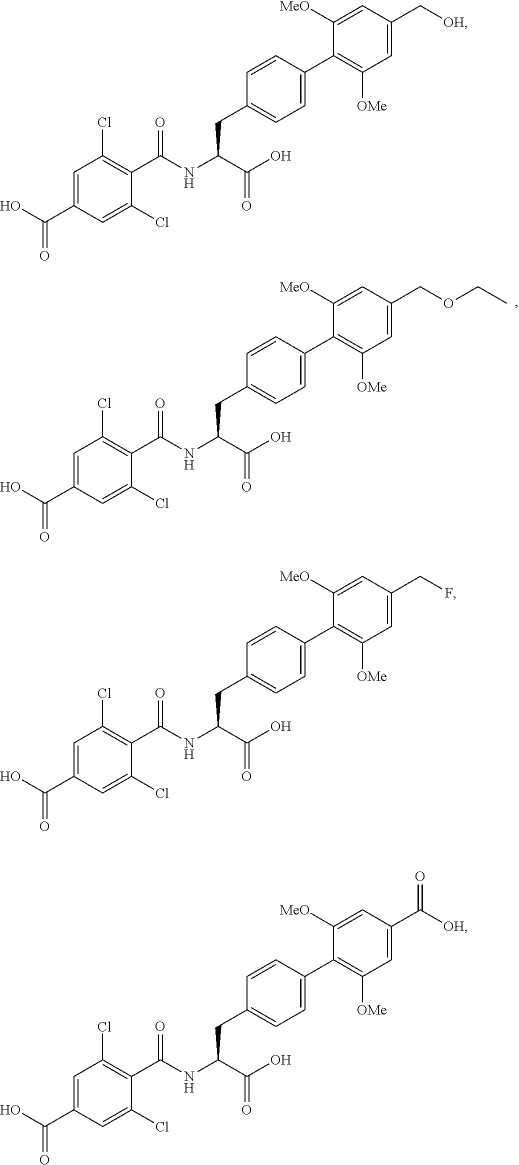 [SMILES: COc1cc(CO)cc(OC)c1-c1ccc(C[C@H](NC(=O)c2c(Cl)cc(C(=O)O)cc2Cl)C(=O)O)cc1 ; CCOCc1cc(OC)c(-c2ccc(C[C@H](NC(=O)c3c(Cl)cc(C(=O)O)cc3Cl)C(=O)O)cc2)c(OC)c1 ; COc1cc(CF)cc(OC)c1-c1ccc(C[C@H](NC(=O)c2c(Cl)cc(C(=O)O)cc2Cl)C(=O)O)cc1 ; COc1cc(C(=O)O)cc(OC)c1-c1ccc(C[C@H](NC(=O)c2c(Cl)cc(C(=O)O)cc2Cl)C(=O)O)cc1]

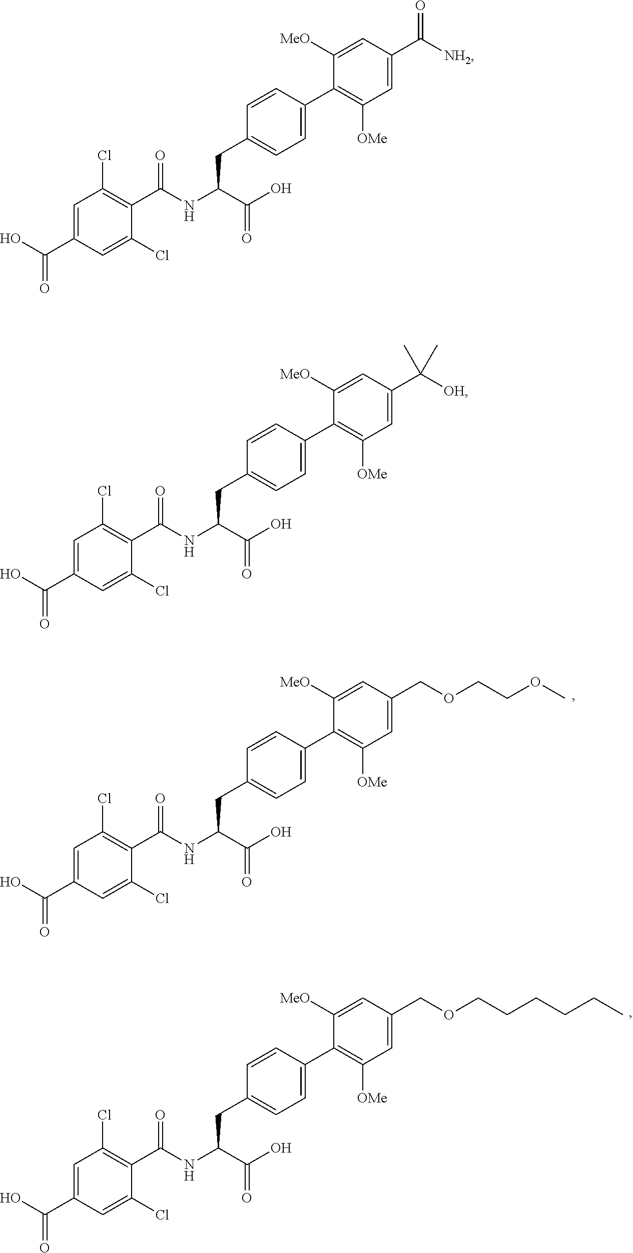 [SMILES: COc1cc(C(N)=O)cc(OC)c1-c1ccc(C[C@H](NC(=O)c2c(Cl)cc(C(=O)O)cc2Cl)C(=O)O)cc1 ; COc1cc(C(C)(C)O)cc(OC)c1-c1ccc(C[C@H](NC(=O)c2c(Cl)cc(C(=O)O)cc2Cl)C(=O)O)cc1 ; COCCOCc1cc(OC)c(-c2ccc(C[C@H](NC(=O)c3c(Cl)cc(C(=O)O)cc3Cl)C(=O)O)cc2)c(OC)c1 ; CCCCCCOCc1cc(OC)c(-c2ccc(C[C@H](NC(=O)c3c(Cl)cc(C(=O)O)cc3Cl)C(=O)O)cc2)c(OC)c1]

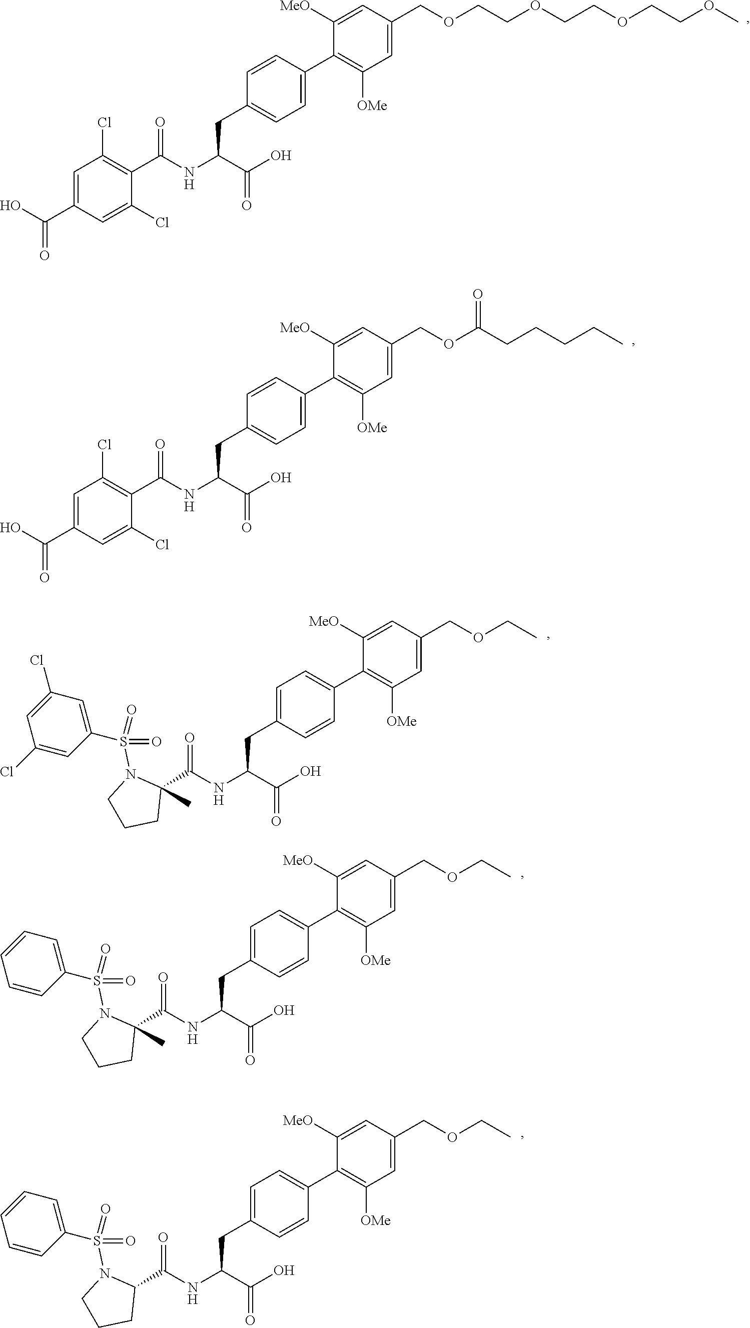 [SMILES: COCCOCCOCCOCc1cc(OC)c(-c2ccc(C[C@H](NC(=O)c3c(Cl)cc(C(=O)O)cc3Cl)C(=O)O)cc2)c(OC)c1 ; CCCCCC(=O)OCc1cc(OC)c(-c2ccc(C[C@H](NC(=O)c3c(Cl)cc(C(=O)O)cc3Cl)C(=O)O)cc2)c(OC)c1 ; CCOCc1cc(OC)c(-c2ccc(C[C@H](NC(=O)[C@]3(C)CCCN3S(=O)(=O)c3cc(Cl)cc(Cl)c3)C(=O)O)cc2)c(OC)c1 ; CCOCc1cc(OC)c(-c2ccc(C[C@H](NC(=O)[C@]3(C)CCCN3S(=O)(=O)c3ccccc3)C(=O)O)cc2)c(OC)c1 ; CCOCc1cc(OC)c(-c2ccc(C[C@H](NC(=O)[C@@H]3CCCN3S(=O)(=O)c3ccccc3)C(=O)O)cc2)c(OC)c1]

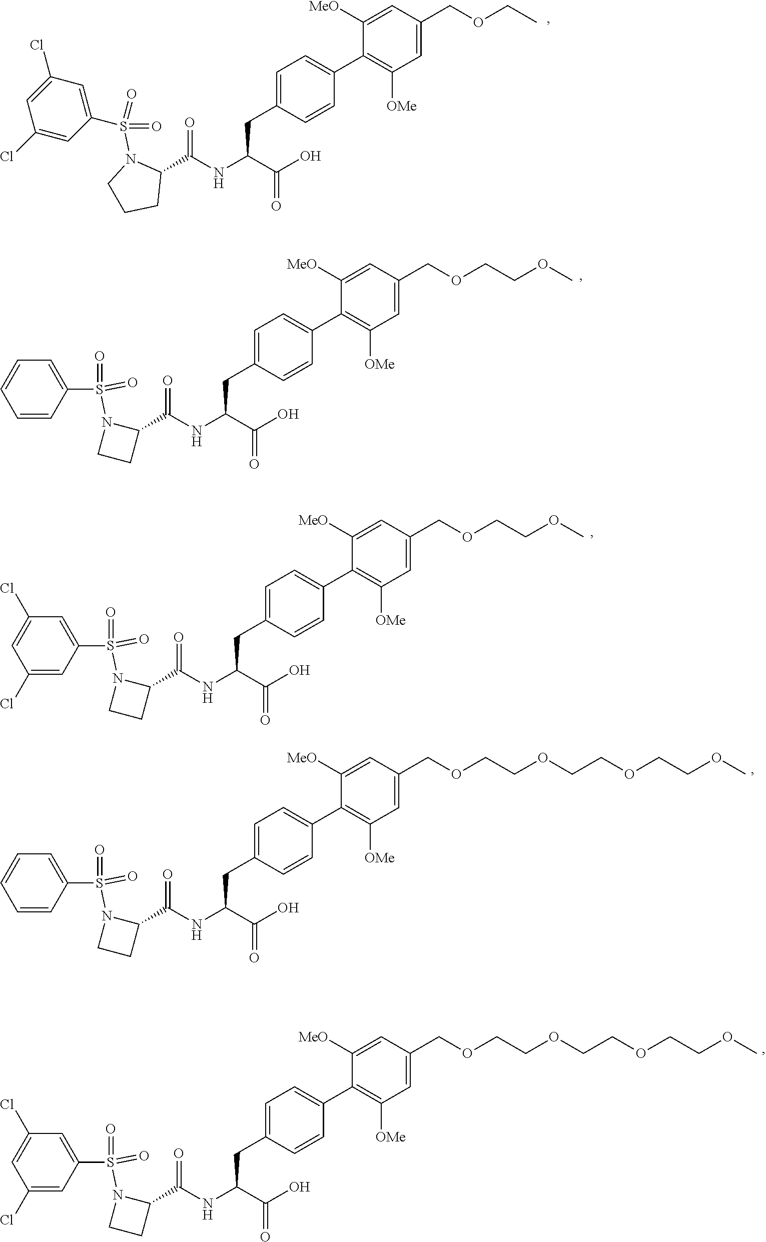 [SMILES: CCOCc1cc(OC)c(-c2ccc(C[C@H](NC(=O)[C@@H]3CCCN3S(=O)(=O)c3cc(Cl)cc(Cl)c3)C(=O)O)cc2)c(OC)c1 ; COCCOCc1cc(OC)c(-c2ccc(C[C@H](NC(=O)[C@@H]3CCN3S(=O)(=O)c3ccccc3)C(=O)O)cc2)c(OC)c1 ; COCCOCc1cc(OC)c(-c2ccc(C[C@H](NC(=O)[C@@H]3CCN3S(=O)(=O)c3cc(Cl)cc(Cl)c3)C(=O)O)cc2)c(OC)c1 ; COCCOCCOCCOCc1cc(OC)c(-c2ccc(C[C@H](NC(=O)[C@@H]3CCN3S(=O)(=O)c3ccccc3)C(=O)O)cc2)c(OC)c1 ; COCCOCCOCCOCc1cc(OC)c(-c2ccc(C[C@H](NC(=O)[C@@H]3CCN3S(=O)(=O)c3cc(Cl)cc(Cl)c3)C(=O)O)cc2)c(OC)c1]

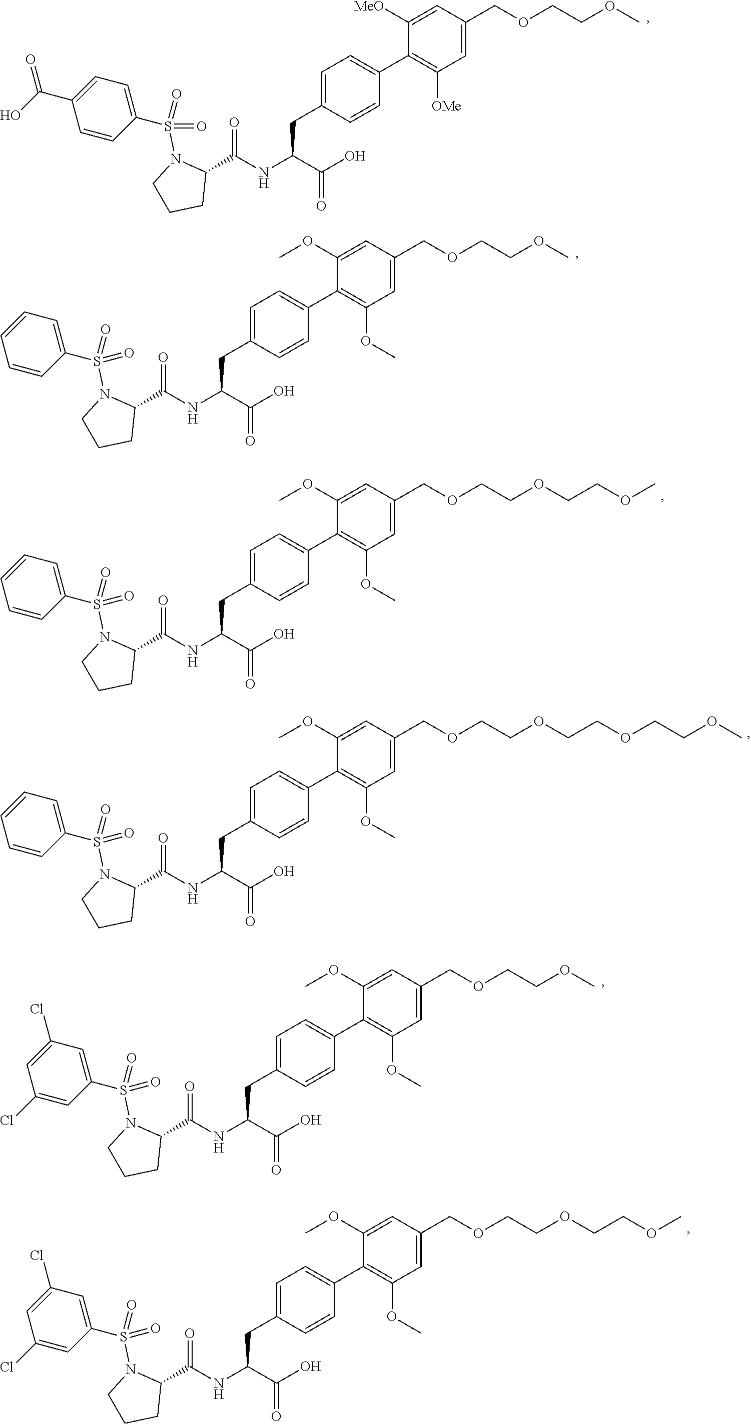

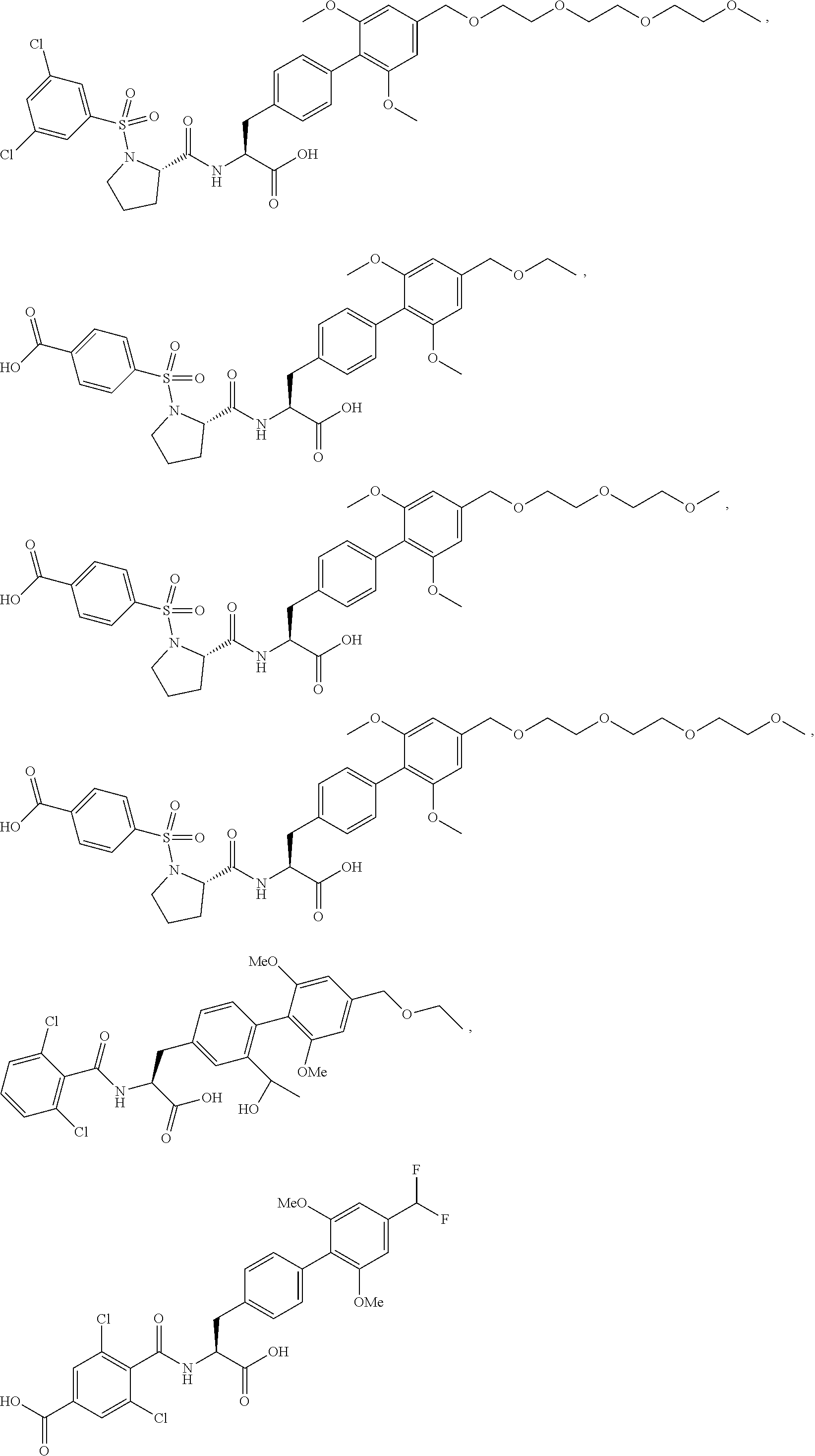

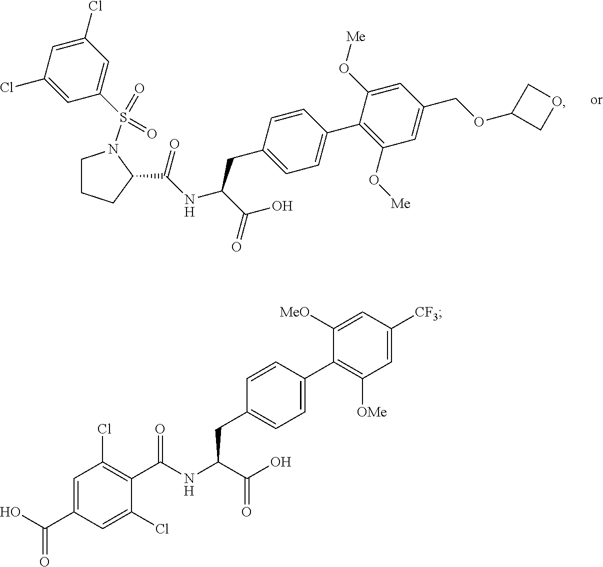 [SMILES: COc1cc(COC2COC2)cc(OC)c1-c1ccc(C[C@H](NC(=O)[C@@H]2CCCN2S(=O)(=O)c2cc(Cl)cc(Cl)c2)C(=O)O)cc1 ; "or" ; COc1cc(C(F)(F)F)cc(OC)c1-c1ccc(C[C@H](NC(=O)c2c(Cl)cc(C(=O)O)cc2Cl)C(=O)O)cc1]

or a pharmaceutically salt thereof.

In some aspects, the present disclosure provides pharmaceutical compositions comprising:

(A) a compound disclosed herein; and
(B) a pharmaceutically acceptable excipient.

In some embodiments, the pharmaceutical composition is formulated for administration: orally, intraadiposally, intraarterially, intraarticularly, intracranially, intradermally, intralesionally, intramuscularly, intranasally, intraocularly, intrapericardially, intraperitoneally, intrapleurally, intraprostatically, intrarectally, intrathecally, intratracheally, intratumorally, intraumbilically, intravaginally, intravenously, intravesicularlly, intravitreally, liposomally, locally, mucosally, parenterally, rectally, subconjunctival, subcutaneously, sublingually, topically, transbuccally, transdermally, vaginally, in cremes, in lipid compositions, via a catheter, via a lavage, via continuous infusion, via infusion, via inhalation, via injection, via local delivery, or via localized perfusion.

In some embodiments, the pharmaceutical composition is formulated for oral administration, intraarterial administration, intraperitoneal administration, intravenous administration, or subcutaneous administration. In some embodiments, the pharmaceutical composition is formulated for administration via intravenous infusion. In other embodiments, the pharmaceutical composition is formulated for subcutaneous administration. In some embodiments, the pharmaceutical composition is formulated as a unit dose.

In some aspects, the present disclosure provides methods of treating a disease or disorder in a patient comprising administering to the patient a therapeutically effective amount of a compound or composition disclosed herein. In some embodiments, the disease or disorder is associated with integrin α₄β₁. In other embodiments, the disease or disorder is associated with inflammation. In yet other embodiments, the disease or disorder is an autoimmune disorder. In still other embodiments, the disease or disorder is associated with hematopoietic stem cells such as LSK-SLAM cells. In yet other embodiments, the disease or disorder is cancer or a reduced blood cell count such as reduced blood cell count resulting from a therapy for cancer. In some embodiments, the disease or disorder is a reduced blood cell count resulting from a therapy for cancer such as chemotherapy or radiation therapy. In other embodiments, the disease or disorder is cancer. In some embodiments, the compound or composition results in improved efficacy of the chemotherapy or radiotherapy.

In some aspects, the present disclosure provides methods of inducing the mobilization of hematopoietic stem cells or progenitor cells comprising contacting the hematopoietic stem cells or progenitor cells with an effective amount of a compound or composition disclosed herein. In some embodiments, the method is ex vivo or in vitro. In some embodiments, method is in vivo.

In some aspects, the present disclosure provides methods of collecting hematopoietic stem cells or progenitor cells from a patient comprising:
(A) administering to the patient a compound or composition disclosed herein in an amount sufficient to mobilize hematopoietic stem cells or progenitor cells to the peripheral blood of the patient; and
(B) subsequently drawing peripheral blood from the patient to collect the hematopoietic stem cells or progenitor cells.

In some aspects, the present disclosure provides methods of collecting hematopoietic stem cells or progenitor cells from a patient who has been administered a compound or composition disclosed herein in an amount sufficient to mobilize hematopoietic stem cells or progenitor cells to the peripheral blood of the patient comprising subsequently drawing peripheral blood from the patient to collect the hematopoietic stem cells or progenitor cells.

In other aspects, the present disclosure provides methods of improving the harvest of hematopoietic stem cells or progenitor cells comprising administering to a patient a therapeutically effective amount of a compound or composition disclosed herein.

In yet other aspects, the present disclosure provides methods of transplanting to a patient hematopoietic stem cells or progenitor cells comprising:
(A) administering to the patient a compound or composition disclosed herein;
(B) collecting hematopoietic stem cells or progenitor cells from the patient;
(C) transplanting the hematopoietic stem cells or progenitor cells in the patient.

In some aspects, the present disclosure provides methods of transplanting to a patient hematopoietic stem cells or progenitor cells comprising transplanting the hematopoietic stem cells or progenitor cells collected from the patient who has been administered a therapeutically effective amount of a compound or composition disclosed herein.

In some aspects, the present disclosure provides methods of transplanting hematopoietic stem cells or progenitor cells comprising:
(A) administering to a first patient a compound or composition described herein;
(B) collecting hematopoietic stem cells or progenitor cells from the first patient;
(C) transplanting the hematopoietic stem cells or progenitor cells in the second patient.

In some aspects, the present disclosure provides methods invention of transplanting hematopoietic stem cells or progenitor cells comprising transplanting the hematopoietic stem cells or progenitor cells collected from a first patient who has been administered a therapeutically effective amount of a compound or composition disclosed herein to a second patient.

In some embodiments, the hematopoietic stem cells are collected from the patient before an event which results in a reduction of the amount of the patient's hematopoietic stem cells or progenitor cells. In some embodiments, the hematopoietic stem cells or progenitor cells are transplanted after an event which results in a reduction of the amount of the patient's hematopoietic stem cells or progenitor cells. In some embodiments, the first patient is a compatible hematopoietic stem cell donor. In some embodiments, the hematopoietic stem cells or progenitor cells are LSK-SLAM cells.

In some aspects, the present disclosure provides methods of improving the effectiveness of a treatment of cancer in a patient administered a chemotherapy or a radiotherapy comprising:
(A) administering to the patient a therapeutically effective amount of a compound or composition disclosed herein;
(B) administering a chemotherapy or a radiotherapy to the patient.

In some aspects, the present disclosure provides methods of improving the effectiveness of a treatment of cancer in patient who has been administered a chemotherapy or radiotherapy and a compound or composition disclosed herein.

In some embodiments, the methods comprise administering the compound or composition once. In other embodiments, the methods comprise administering the compound or composition two or more times. In some embodiments, the compound or composition is administered intravenously. In other embodiments, the compound or composition is administered subcutaneously. In some embodiments, the patient is a mammal, such as a human.

Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description. Note that simply because a particular compound is ascribed to one particular generic formula does not mean that it cannot also belong to another generic formula.

### (c) Description of Illustrative Embodiments

Disclosed herein are new compounds and compositions which act as integrin antagonists (i.e. inhibitors) of, for example, α4β1 integrin (VLA-4), methods for their manufacture, and methods for their use, including for the treatment and/or prevention of disease. In some embodiments, these compounds may be used in improving the harvest of hematopoietic stem cells or progenitor cells or to enhance an anti-cancer therapy.

(i) Compounds and Synthetic Methods

In some embodiments, the compounds of the present disclosure include the compounds described in the Examples and claims listed below. All the methods described herein can be further modified and optimized using the principles and techniques of organic chemistry as applied by a person skilled in the art. Such principles and techniques are taught, for example, in March's Advanced Organic Chemistry: Reactions, Mechanisms, and Structure (2007), which is incorporated by reference herein.

Compounds employed in methods of the disclosure may contain one or more asymmetrically-substituted carbon or nitrogen atoms, and may be isolated in optically active or racemic form. Thus, all chiral, diastereomeric, racemic form, epimeric form, and all geometric isomeric forms of a structure are intended, unless the specific stereochemistry or isomeric form is specifically indicated. Compounds may occur as racemates and racemic mixtures, single enantiomers, diastereomeric mixtures and individual diastereomers. In some embodiments, a single diastereomer is obtained. The chiral centers of the compounds of the present disclosure can have the S or the R configuration, as defined by the IUPAC 1974 Recommendations. For example, mixtures of stereoisomers may be separated using the techniques taught in the Examples section below, as well as modifications thereof. Tautomeric forms are also included as well as pharmaceutically acceptable salts of such isomers and tautomers.

Atoms making up the compounds of the present disclosure are intended to include all isotopic forms of such atoms. Compounds of the present disclosure include those with one or more atoms that have been isotopically modified or enriched, in particular those with pharmaceutically acceptable isotopes or those useful for pharmaceutically research. Isotopes, as used herein, include those atoms having the same atomic number but different mass numbers. By way of general example and without limitation, isotopes of hydrogen include deuterium and tritium, and isotopes of carbon include ¹³C and ¹⁴C. Similarly, it is contemplated that one or more carbon atom(s) of a compound of the present disclosure may be replaced by a silicon atom(s). Furthermore, it is contemplated that one or more oxygen atom(s) of a compound of the present disclosure may be replaced by a sulfur or selenium atom(s).

Compounds of the present disclosure may also exist in prodrug form. Since prodrugs are known to enhance numerous desirable qualities of pharmaceuticals (e.g., solubility, bioavailability, manufacturing, etc.), the compounds employed in some methods of the disclosure may, if desired, be delivered in prodrug form. Thus, the disclosure contemplates prodrugs of compounds of the present disclosure as well as methods of delivering prodrugs. Prodrugs of the compounds employed in the disclosure may be prepared by modifying functional groups present in the compound in such a way that the modifications are cleaved, either in routine manipulation or in vivo, to the parent compound. Accordingly, prodrugs include, for example, compounds described herein in which a hydroxy, amino, or carboxy group is bonded to any group that, when the prodrug is administered to a subject, cleaves to form a hydroxy, amino, or carboxylic acid, respectively. Additional details regarding pro-drugs may be found in Smith and Williams, 1988, the contents of which are hereby incorporated by reference.

It should be recognized that the particular anion or cation forming a part of any salt of this disclosure is not critical, so long as the salt, as a whole, is pharmacologically acceptable. Additional examples of pharmaceutically acceptable salts and their methods of preparation and use are presented in Handbook of Pharmaceutical Salts: Properties, and Use (2002), which is incorporated herein by reference.

It should be further recognized that the compounds of the present disclosure include those that have been further modified to comprise substituents that are convertible to hydroxy in vivo. This includes those groups that may be convertible to a hydrogen atom by enzymological or chemical means including, but not limited to, hydrolysis and hydrogenolysis. Examples include hydrolyzable groups, such as acyl groups, groups having an oxycarbonyl group, amino acid residues, peptide residues, o-nitrophenylsulfenyl, trimethylsilyl, tetrahydropyranyl, diphenylphosphinyl, and the like. Examples of acyl groups include formyl, acetyl, trifluoroacetyl, and the like. Examples of groups having an oxycarbonyl group include ethoxycarbonyl, tert-butoxycarbonyl (—C(O)OC(CH₃)₃, Boc), benzyloxycarbonyl, p-methoxybenzyloxycarbonyl, vinyloxycarbonyl, R-(p-toluenesulfonyl)ethoxycarbonyl, and the like. Suitable amino acid residues include, but are not limited to, residues of Gly (glycine), Ala (alanine), Arg (arginine), Asn (asparagine), Asp (aspartic acid), Cys (cysteine), Glu (glutamic acid), His (histidine), lie (isoleucine), Leu (leucine), Lys (lysine), Met (methionine), Phe (phenylalanine), Pro (proline), Ser (serine), Thr (threonine), Trp (tryptophan), Tyr (tyrosine), Val (valine), Nva (norvaline), Hse (homoserine), 4-Hyp (4-hydroxyproline), 5-Hyl (5-hydroxylysine), Orn (ornithine) and R-Ala. Examples of suitable amino acid residues also include amino acid residues that are protected with a protecting group. Examples of suitable protecting groups include those typically employed in peptide synthesis, including acyl groups (such as formyl and acetyl), arylmethoxycarbonyl groups (such as benzyloxycarbonyl and p-nitrobenzyloxycarbonyl), tert-butoxycarbonyl groups (—C(O)OC(CH₃)₃, Boc), and the like. Suitable peptide residues include peptide residues comprising two to five amino acid residues. The residues of these amino acids or peptides can be present in stereochemical configurations of the D-form, the L-form or mixtures thereof. In addition, the amino acid or peptide residue may have an asymmetric carbon atom. Examples of suitable amino acid residues having an asymmetric carbon atom include residues of Ala, Leu, Phe, Trp, Nva, Val, Met, Ser, Lys, Thr and Tyr. Peptide residues having an asymmetric carbon atom include peptide residues having one or more constituent amino acid residues having an asymmetric carbon atom. Examples of suitable amino acid protecting groups include those typically employed in peptide synthesis, including acyl groups (such as formyl and acetyl), arylmethoxycarbonyl groups (such as benzyloxycarbonyl and p-nitrobenzyloxycarbonyl), tert-butoxycarbonyl groups (—C(O)OC(CH₃)₃), and the like. Other examples of substituents “convertible to hydroxy in vivo” include reductively eliminable hydrogenolyzable groups. Examples of suitable reductively eliminable hydrogenolyzable groups include, but are not limited to, arylsulfonyl groups (such as o-toluenesulfonyl); methyl groups substituted with phenyl or benzyloxy (such as benzyl, trityl and benzyloxymethyl); arylmethoxycarbonyl groups (such as benzyloxycarbonyl and o-methoxy-benzyloxycarbonyl); and haloethoxycarbonyl groups (such as β,β,β-trichloroethoxycarbonyl and β-iodoethoxycarbonyl).

Compounds of the disclosure may also have the advantage that they may be more efficacious than, be less toxic than, be longer acting than, be more potent than, produce fewer side effects than, be more easily absorbed than, and/or have a better pharmacokinetic profile (e.g., higher oral bioavailability and/or lower clearance) than, and/or have other useful pharmacological, physical, or chemical properties over, compounds known in the prior art, whether for use in the indications stated herein or otherwise.

(ii) Biological Activity

It is another object of the disclosure to provide pharmaceutical compositions comprising compounds described above. These compounds and pharmaceutical compositions may be used to improve the harvest of hematopoietic stem cells or progenitor cells. Additionally, the compounds or compositions may be used to elevate the circulation of hematopoietic progenitor and/or stem cells, improve the collection of hematopoietic stem cells or progenitor cells for a transfusion, increase the sensitization of an anti-cancer therapy such as a chemotherapeutic or radiotherapy, or mobilize pre-cancerous or cancerous cells into the peripheral blood which may increase their sensitivity to an anti-cancer therapy.

Hematopoietic stem cell transplant (HSCT) is used to facilitate repopulation of healthy bone marrow and immune system cells after high-dose chemotherapy treatment for cancers such as Hodgkin's and non-Hodgkin's lymphoma, multiple myeloma, and leukemia. In HSCT, hematopoietic stem/progenitor cells (HSPCs) are collected from the patient's blood, harvested, frozen and then stored while the patient receives high-dose chemotherapy and/or radiation therapy. Successful HSCT requires the intravenous infusion of a minimum number of 2×10⁶ CD34+ stem cells/kg body weight; however, a dose of 5×10⁶ CD34+ cells/kg is considered preferable for early and long term multilineage engraftment.

Stem cells harvested from peripheral blood are the most commonly used graft source in HSCT. While granulocyte colony-stimulating factor (G-CSF) is the most frequently used agent for stem cell mobilization, the use of G-CSF alone results in suboptimal stem cell yields in a significant proportion of patients. Plerixafor (AMD3100), a small molecule CXCR4 antagonist, in combination with G-CSF increases total CD34+ HSPCs compared to G-CSF alone and is FDA approved for stem cell mobilization in Non-Hodgkin's lymphoma and multiple myeloma. However, a significant disadvantage of plerixafor is cost, adding $25,567 per patient compared to G-CSF alone. Furthermore, up to 24% of patients receiving plerixafor and G-CSF still fail to collect ≥2×10⁶ CD34+ cells/kg in 4 days of apheresis. Recent economic analysis has determined that reducing apheresis by 1 day has the potential to decrease medical costs by $6,600. Thus improved/alternative mobilizing agents and strategies are needed.

Mechanistic studies have shown that the integrin α4β1 (VLA-4) plays an important role in the retention of HSPCs within the bone marrow (BM) microenvironment. HSPC mobilization has been achieved by disrupting the integrin α4β1/VCAM-1 axis with antibodies against integrin α4β1 or VCAM-1. Preclinical mouse studies in the DiPersio laboratory have shown that administration of the small molecule inhibitor of integrin α4β1, BIO5192, results in the rapid and reversible mobilization of HSPCs into the peripheral circulation with maximum mobilization occurring within 30 to 60 minutes and returning to baseline within 4 hours. A superior treatment could be envisioned wherein a patient receives an integrin α4β1 antagonist to continually inhibit integrin α4β1 over the course of ˜4 hours (average duration of CD34+ stem cell apheresis procedures), maximizing the mobilization of HSPCs that can be collected by apheresis during the same day of treatment.

BIO5192 is a potent small molecule inhibitor of integrin α4β1 and has demonstrated efficacy in mobilizing HSPC's in mice. However, BIO5192 has poor aqueous solubility, bioavailability, and pharmacokinetic properties and therefore has not been developed clinically. A simpler, more soluble integrin α4β1 antagonist is firategrast. Firategrast has been tested in clinical trials for the treatment of multiple sclerosis and has demonstrated efficacy in mobilizing HSPC's in mice but to a significantly lesser extent than BIO5192 and at much higher doses. Neither of these currently available integrin α4β1 antagonists have the appropriate properties to be useful for HSCT. These compounds or compositions may also have the added advantage that the compositions or methods result in the mobilization in higher numbers, begin mobilization in a shorter period of time, over a more prolonged period of time, or mobilize increased numbers of early progenitor and/or stem cells, LSK-SLAM cells, CFU-C cells, or other progenitor and/or stem cells which are competent to achieve a successful engraftment into the patient.

(iii) Therapeutic Methods

The present disclosure relates to the fields of pharmaceuticals, medicine and cell biology. In another aspect, this disclosure provides methods of inhibiting or antagonizing VLA-4 using one or more of the compounds disclosed herein, as well as pharmaceutical compositions thereof.

In one aspect, the compounds and compositions described herein may be used to increase the harvest of HSPCs for a variety of different applications. These compounds and compositions may be used to treat a patient who requires a transplantation. Alternatively, the compounds and compositions may be used to treat a patient who does not require a transplantation. The patient who needs a transplant of HSPCs requires either an allogenic, autologous, or tandem transplant of HSPCs. In some embodiments, the HSPCs may be used in either allogenic or autologous transplants. In another aspect, the present compounds and compositions described herein may be used to improve the circulation of cells to tissues which need repair. The increased circulation of HSPCs may be used to improve the repair of the target tissue in the patient.

If the HSPCs are harvested, these cells may be returned to the donor patient (autologous transplant) or may be donated to another patient that is sufficiently compatible to prevent rejection (allogeneic transplant). One non-limiting application of autologous transplantation is in combination with radiation or chemotherapy in patients bearing tumors since the radiotherapeutic or chemotherapeutic methods deplete the patient's normal cells. In this application, the patient's cells may be harvested prior to or during the therapeutic treatments, fractionated if necessary, cultured and optionally expanded, and then returned to the patient to restore the damaged immune system depleted by the therapy. Allogeneic recipients may receive the cells for the same purpose, or may have a condition that may be benefited by enhancing their hematopoietic systems. In a typical protocol for these types of transplants, the mobilized cells are collected from the donor by, for example, apheresis and then stored/cultured/expanded/fractionated as desired. In some embodiments, the compounds and compositions described herein may result in the need for apheresis being eliminated.

In some aspects, the present compounds and compositions described herein may be used to increase the circulation of pre-cancerous or cancerous cells out of the bone marrow into the peripheral blood. Without wishing to be bound by any theory, it is believed that increasing the circulation of pre-cancerous or cancerous cells out of the bone marrow may increase the effectiveness of an anti-cancer therapy. In particular, these compounds and compositions may be used to treat patients who have or are at risk of a hematopoietic malignancy such as lymphoma, myeloma, or leukemia. The compounds and compositions described herein may be administered or employed prior to, during, or subsequent to the anti-cancer therapy. Two non-limiting examples of anti-cancer therapies that may be used in the methods described herein or conjunction with the compounds and compositions described herein include chemotherapeutic agents or radiotherapy.

In another aspect, the compounds and compositions described herein may be used to decrease inflammation which may result in increasing tissue repair. Thus, the compounds and compositions described herein may be used to treat graft versus host disease. Additionally, these compounds and compositions may be used to treat diseases or disorders associated with cell adhesion-mediated inflammatory pathways. Some non-limiting examples of cell adhesion-mediated inflammatory pathologies include asthma, multiple sclerosis, rheumatoid arthritis, atherosclerosis, and inflammatory bowel disease.

Such pharmaceutical compositions further comprise one or more non-toxic, pharmaceutically acceptable carriers and/or diluents and/or adjuvants (collectively referred to herein as “carrier” materials) and if desired other active ingredients. These methods may be used to treat a blood disease or disorder such as sickle cell anemia or as a part of hematopoietic stem cell therapy to promote the development of stem cells. In some embodiments, the compound is administered as part of a pharmaceutical composition further comprising a pharmaceutically acceptable carrier. In some embodiments, the compounds and/or pharmaceutical compositions thereof may be administered orally, parenterally, or by inhalation spray, or topically in unit dosage formulations containing conventional pharmaceutically acceptable carriers, adjuvants and vehicles. The term parenteral as used herein includes, for example, subcutaneous, intravenous, intramuscular, intrasternal, infusion techniques or intraperitoneally. In some embodiments, the compounds of the present disclosure are administered by any suitable route in the form of a pharmaceutical composition adapted to such a route, and in a dose effective for the treatment intended. Therapeutically effective doses of the compounds required to prevent or arrest the progress of or to treat a medical condition are readily ascertained by one of ordinary skill in the art using preclinical and clinical approaches familiar to the medicinal arts.

Based upon standard laboratory experimental techniques and procedures well known and appreciated by those skilled in the art, as well as comparisons with compounds of known usefulness, the compounds described above can be used in the treatment of patients suffering from the above pathological conditions. One skilled in the art will recognize that selection of the most appropriate compound of the disclosure is within the ability of one with ordinary skill in the art and will depend on a variety of factors including assessment of results obtained in standard assay and animal models.

In several aspects of the present disclosure, the compounds provided herein may be used in a variety of biological, prophylactic or therapeutic areas, including those in wherein VLA-4 plays a role.

(iv) Pharmaceutical Formulations and Routes of Administration

For administration to an animal especially a mammal in need of such treatment, the compounds in a therapeutically effective amount are ordinarily combined with one or more excipients appropriate to the indicated route of administration. The compounds of the present disclosure are contemplated to be formulated in a manner amenable to treatment of a veterinary patient as well as a human patient. In some embodiments, the veterinary patient may be an avian such as chicken, turkey, or duck, a companion animal such as a cat or dog, livestock animals such as a cow, horse, pig, or goat, zoo animals, and wild animals. The compounds may be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia, sodium alginate, polyvinylpyrrolidone, and/or polyvinyl alcohol, and tableted or encapsulated for convenient administration. Alternatively, the compounds may be dissolved in water, polyethylene glycol, propylene glycol, ethanol, corn oil, cottonseed oil, peanut oil, sesame oil, benzyl alcohol, sodium chloride, and/or various buffers. Other excipients and modes of administration are well and widely known in the pharmaceutical art and may be adapted to the type of animal being treated. Description of potential administration routes which may be used to formulate the compositions described herein include those taught in Remington's Pharmaceutical Sciences, which is incorporated herein by reference.

The pharmaceutical compositions useful in the present disclosure may be subjected to conventional pharmaceutical operations such as sterilization and/or may contain conventional pharmaceutical carriers and excipients such as preservatives, stabilizers, wetting agents, emulsifiers, buffers, etc.

The compounds of the present disclosure may be administered by a variety of methods, e.g., orally or by injection (e.g. subcutaneous, intravenous, intraperitoneal, etc.). Depending on the route of administration, the active compounds may be coated in a material to protect the compound from the action of acids and other natural conditions which may inactivate the compound. They may also be administered by continuous perfusion/infusion of a disease or wound site.

To administer the therapeutic compound by other than parenteral administration, it may be necessary to coat the compound with, or co-administer the compound with, a material to prevent its inactivation. For example, the therapeutic compound may be administered to a patient in an appropriate carrier, for example, liposomes, or a diluent. Pharmaceutically acceptable diluents include saline and aqueous buffer solutions. Liposomes include water-in-oil-in-water CGF emulsions as well as conventional liposomes.

The therapeutic compound may also be administered parenterally, intraperitoneally, intramuscularly, intraarterially, intraspinally, or intracerebrally. Dispersions can be prepared in glycerol, liquid polyethylene glycols, and mixtures thereof and in oils. Under ordinary conditions of storage and use, these preparations may contain a preservative to prevent the growth of microorganisms.

Pharmaceutical compositions may be suitable for injectable use include sterile aqueous solutions (where water soluble) or dispersions and sterile powders for the extemporaneous preparation of sterile injectable solutions or dispersion. In all cases, the composition must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (such as, glycerol, propylene glycol, and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it may be useful to include isotonic agents, for example, sugars, sodium chloride, or polyalcohols such as mannitol and sorbitol, in the composition. Prolonged absorption of the injectable compositions can be brought about by including in the composition an agent which delays absorption, for example, aluminum monostearate or gelatin.

Sterile injectable solutions can be prepared by incorporating the therapeutic compound in the required amount in an appropriate solvent with one or a combination of ingredients enumerated above, as required, followed by filtered sterilization. Generally, dispersions are prepared by incorporating the therapeutic compound into a sterile carrier which contains a basic dispersion medium and the required other ingredients from those enumerated above. In the case of sterile powders for the preparation of sterile injectable solutions, the methods of preparation include vacuum drying and freeze-drying which yields a powder of the active ingredient (i.e., the therapeutic compound) plus any additional desired ingredient from a previously sterile-filtered solution thereof.

The therapeutic compound can be orally administered, for example, with an inert diluent or an assimilable edible carrier. The therapeutic compound and other ingredients may also be enclosed in a hard or soft shell gelatin capsule, compressed into tablets, or incorporated directly into the subject's diet. For oral therapeutic administration, the therapeutic compound may be incorporated with excipients and used in the form of ingestible tablets, buccal tablets, troches, capsules, elixirs, suspensions, syrups, wafers, and the like. The percentage of the therapeutic compound in the compositions and preparations may, of course, be varied. The amount of the therapeutic compound in such therapeutically useful compositions is such that a suitable dosage will be obtained.

It is especially advantageous to formulate parenteral compositions in dosage unit form for ease of administration and uniformity of dosage. Dosage unit form as used herein refers to physically discrete units suited as unitary dosages for the subjects to be treated; each unit containing a predetermined quantity of therapeutic compound calculated to produce the desired therapeutic effect in association with the required pharmaceutical carrier. The specification for the dosage unit forms of the disclosure are dictated by and directly dependent on (a) the unique characteristics of the therapeutic compound and the particular therapeutic effect to be achieved, and (b) the limitations inherent in the art of compounding such a therapeutic compound for the treatment of a selected condition in a patient.

The therapeutic compound may also be administered topically to the skin, eye, or mucosa. Alternatively, if local delivery to the lungs is desired the therapeutic compound may be administered by inhalation in a dry-powder or aerosol formulation.

Active compounds are administered at a therapeutically effective dosage sufficient to treat a condition associated with a condition in a patient. For example, the efficacy of a compound can be evaluated in an animal model system that may be predictive of efficacy in treating the disease in a human or another animal, such as the model systems shown in the examples and drawings.

An effective dose range of a therapeutic can be extrapolated from effective doses determined in animal studies for a variety of different animals. In general a human equivalent dose (HED) in mg/kg can be calculated in accordance with the following formula (see, e.g., Reagan-Shaw et al., FASEB J., 22(3):659-661, 2008, which is incorporated herein by reference):

HED (mg/kg)=Animal dose (mg/kg)×(Animal Kₘ/Human Kₘ)

Use of the Kₘ factors in conversion results in more accurate HED values, which are based on body surface area (BSA) rather than only on body mass. Kₘ values for humans and various animals are well known. For example, the Kₘ for an average 60 kg human (with a BSA of 1.6 m²) is 37, whereas a 20 kg child (BSA 0.8 m²) would have a Kₘ of 25. Kₘ for some relevant animal models are also well known, including: mice Kₘ of 3 (given a weight of 0.02 kg and BSA of 0.007); hamster Kₘ of 5 (given a weight of 0.08 kg and BSA of 0.02); rat Kₘ of 6 (given a weight of 0.15 kg and BSA of 0.025) and monkey Kₘ of 12 (given a weight of 3 kg and BSA of 0.24).

Precise amounts of the therapeutic composition depend on the judgment of the practitioner and are peculiar to each individual. Nonetheless, a calculated HED dose provides a general guide. Other factors affecting the dose include the physical and clinical state of the patient, the route of administration, the intended goal of treatment and the potency, stability and toxicity of the particular therapeutic formulation.

The actual dosage amount of a compound of the present disclosure or composition comprising a compound of the present disclosure administered to a subject may be determined by physical and physiological factors such as type of animal treated, age, sex, body weight, severity of condition, the type of disease being treated, previous or concurrent therapeutic interventions, idiopathy of the subject and on the route of administration. These factors may be determined by a skilled artisan. The practitioner responsible for administration will typically determine the concentration of active ingredient(s) in a composition and appropriate dose(s) for the individual subject. The dosage may be adjusted by the individual physician in the event of any complication.

In some embodiments, the VLA-4 antagonist (i.e. inhibitors) may be administered in an amount from about 1 mg/kg to about 100 mg/kg, or about 1 mg/kg to about 50 mg/kg, or about 1 mg/kg to about 25 mg/kg, or about 1 mg/kg to about 15 mg/kg, or about 1 mg/kg to about 10 mg/kg, or about 1 mg/kg to about 5 mg/kg, or about 3 mg/kg. In some embodiments, a specific VLA-4 inhibitor such as a compound of formula I may be administered in a range of about 1 mg/kg to about 200 mg/kg, or about 50 mg/kg to about 200 mg/kg, or about 50 mg/kg to about 100 mg/kg, or about 75 mg/kg to about 100 mg/kg, or about 100 mg/kg.

The effective amount may be less than 1 mg/kg/day, less than 500 mg/kg/day, less than 250 mg/kg/day, less than 100 mg/kg/day, less than 50 mg/kg/day, less than 25 mg/kg/day or less than 10 mg/kg/day. It may alternatively be in the range of 1 mg/kg/day to 200 mg/kg/day.

In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

In certain embodiments, a pharmaceutical composition of the present disclosure may comprise, for example, at least about 0.1% of a compound of the present disclosure. In other embodiments, the compound of the present disclosure may comprise between about 1% to about 75% of the weight of the unit, or between about 25% to about 60%, for example, and any range derivable therein.

Single or multiple doses of the agents are contemplated. Desired time intervals for delivery of multiple doses can be determined by one of ordinary skill in the art employing no more than routine experimentation. As an example, subjects may be administered two doses daily at approximately 12 hour intervals. In some embodiments, the agent is administered once a day.

The agent(s) may be administered on a routine schedule. As used herein a routine schedule refers to a predetermined designated period of time. The routine schedule may encompass periods of time which are identical or which differ in length, as long as the schedule is predetermined. For instance, the routine schedule may involve administration twice a day, every day, every two days, every three days, every four days, every five days, every six days, a weekly basis, a monthly basis or any set number of days or weeks there-between. Alternatively, the predetermined routine schedule may involve administration on a twice daily basis for the first week, followed by a daily basis for several months, etc. In other embodiments, the disclosure provides that the agent(s) may taken orally and that the timing of which is or is not dependent upon food intake. Thus, for example, the agent can be taken every morning and/or every evening, regardless of when the subject has eaten or will eat.

### II. Integrin Inhibitors in Combination with an Agent which Interacts with a Chemokine Receptor

### (a) Summary

The present disclosure provides methods using a compound which is a VLA-4 antagonist in combination with an agent which interacts with a chemokine receptor (such as a CXCR2 agonist) including methods of use and methods of treatment therewith. Also, provided herein are compositions comprising these two drugs.

In some aspects, the present disclosure provides a method of treating a disease or disorder in a patient comprising administering to the patient a therapeutically effective amount of an agent which interacts with a chemokine receptor and a VLA-4 inhibitor. In some embodiments, the disease or disorder is associated with integrin α₄β₁. In some embodiments, the disease or disorder is associated with hematopoietic stem cells. In some embodiments, the hematopoietic stem cells are LSK-SLAM cells. In some embodiments, the disease or disorder is cancer or a reduced blood cell count such as a reduced blood cell count resulting from a cancer therapy. In some embodiments, the disease or disorder is a reduced blood cell count resulting from a cancer therapy such as chemotherapy or radiation therapy. In some embodiments, the disease or disorder is cancer. In some embodiments, the patient is also administered a chemotherapy or a radiotherapy. In some embodiments, the effective combined amount of an agent which interacts with a chemokine receptor and a VLA-4 inhibitor results in improved efficacy of the chemotherapy or radiotherapy. In some embodiments, the therapeutically effective amount is a therapeutically effective combined amount.

In some aspects, the present disclosure provides a method of inducing the mobilization of hematopoietic stem cells or progenitor cells comprising contacting the hematopoietic stem cells with an effective combined amount of an agent which interacts with a chemokine receptor and a VLA-4 inhibitor. In some embodiments, the method is ex vivo. In other embodiments, the method is in vitro. In yet other embodiments, the method is in vivo.

In some aspects, the present disclosure provides methods of collecting hematopoietic stem cells or progenitor cells from a patient comprising administering to the patient an agent which interacts with a chemokine receptor and a VLA-4 inhibitor disclosed herein in an amount sufficient to mobilize hematopoietic stem cells or progenitor cells to the peripheral blood of the patient and subsequently drawing peripheral blood from the patient to collect the hematopoietic stem cells or progenitor cells.

In some aspects, the present disclosure provides methods of collecting hematopoietic stem cells or progenitor cells from a patient who has been administered an agent which interacts with a chemokine receptor and a VLA-4 inhibitor in an amount sufficient to mobilize hematopoietic stem cells or progenitor cells to the peripheral blood of the patient comprising subsequently drawing peripheral blood from the patient to collect the hematopoietic stem cells or progenitor cells.

In some aspects, the present disclosure provides a method of improving the harvest of hematopoietic stem cells or progenitor cells comprising administering to a patient a therapeutically effective combined amount of an agent which interacts with a chemokine receptor and a VLA-4 inhibitor. In some aspects, the present disclosure provides a method of transplanting hematopoietic stem cells or progenitor cells comprising administering to a first patient a therapeutically effective combined amount of an agent which interacts with a chemokine receptor and a VLA-4 inhibitor, collecting hematopoietic stem cells or progenitor cells from the first patient, and transplanting the hematopoietic stem cells or progenitor cells to a second patient. In some embodiments, the hematopoietic stem cells are collected from the patient before an event which results in a reduction of the amount of the first patient's hematopoietic stem cells or progenitor cells. In some embodiments, the first patient is a compatible hematopoietic stem cell donor.

In some aspects, the present disclosure provides a method of transplanting hematopoietic stem cells or progenitor cells comprising transplanting the hematopoietic stem cells or progenitor cells collected from a first patient who has been administered a therapeutically effective combined amount of an agent which interacts with a chemokine receptor and a VLA-4 inhibitor to a second patient. In some embodiments, the hematopoietic stem cells are collected from the patient before an event which results in a reduction of the amount of the first patient's hematopoietic stem cells or progenitor cells. In some embodiments, the first patient is a compatible hematopoietic stem cell donor.

In some aspects, the present disclosure provides a method of transplanting to a patient hematopoietic stem cells or progenitor cells comprising administering to the patient a therapeutically effective combined amount of an agent which interacts with a chemokine receptor and a VLA-4 inhibitor, collecting hematopoietic stem cells or progenitor cells from the patient, and transplanting the hematopoietic stem cells or progenitor cells in the patient. In some embodiments, the hematopoietic stem cells or progenitor cells are transplanted after an event which results in a reduction of the amount of the patient's hematopoietic stem cells or progenitor cells.

In some aspects, the present disclosure provides a method of transplanting to a patient hematopoietic stem cells or progenitor cells comprising transplanting the hematopoietic stem cells or progenitor cells collected from the patient who has been administered a therapeutically effective combined amount of an agent which interacts with a chemokine receptor and a VLA-4 inhibitor. In some embodiments, the hematopoietic stem cells or progenitor cells are transplanted after an event which results in a reduction of the amount of the patient's hematopoietic stem cells or progenitor cells.

In some aspects, the present disclosure provides a method of improving the effectiveness of a treatment of cancer in a patient administered a chemotherapy or a radiotherapy comprising administering to the patient a therapeutically effective combined amount of an agent which interacts with a chemokine receptor and a VLA-4 inhibitor, and administering a chemotherapy or a radiotherapy to the patient. In some embodiments, the chemotherapy or radiotherapy is administered simultaneously with the agent which interacts with a chemokine receptor and the VLA-4 inhibitor. In some embodiments, the chemotherapy or radiotherapy is administered before the agent which interacts with a chemokine receptor and the VLA-4 inhibitor. In other embodiments, the chemotherapy or radiotherapy is administered after the agent which interacts with a chemokine receptor and the VLA-4 inhibitor.

In some aspects, the present disclosure provides a method of improving the effectiveness of a treatment of cancer in patient who has been or is going to be administered a chemotherapy or radiotherapy and a therapeutically effective combined amount of an agent which interacts with a chemokine receptor and a VLA-4 inhibitor. In some embodiments, the chemotherapy or radiotherapy is administered simultaneously with the agent which interacts with a chemokine receptor and the VLA-4 inhibitor. In some embodiments, the chemotherapy or radiotherapy is administered before the agent which interacts with a chemokine receptor and the VLA-4 inhibitor. In other embodiments, the chemotherapy or radiotherapy is administered after the agent which interacts with a chemokine receptor and the VLA-4 inhibitor.

In some embodiments, the method comprises administering the agent which interacts with a chemokine receptor once. In other embodiments, the method comprises administering the agent which interacts with a chemokine receptor two or more times.

In some embodiments, the method comprises administering the VLA-4 inhibitor once. In other embodiments, the method comprises administering the VLA-4 inhibitor two or more times.

In some embodiments, the VLA-4 inhibitor and the agent which interacts with a chemokine receptor are administered simultaneously. In further embodiments, the method comprises administering a composition comprising the agent which interacts with a chemokine receptor and VLA-4 inhibitor. In other embodiments, the method comprises administering the agent which interacts with a chemokine receptor before administering the VLA-4 inhibitor. In some embodiments, the agent which interacts with a chemokine receptor is administered from 15 minutes to 0 minutes before the VLA-4 inhibitor. In other embodiments, the method comprises administering the agent which interacts with a chemokine receptor after administering the VLA-4 inhibitor.

In some embodiments, the agent which interacts with a chemokine receptor is administered subcutaneously and the VLA-4 inhibitor is administered intravenously. In other embodiments, both the agent which interacts with a chemokine receptor and the VLA-4 inhibitor are administered subcutaneously.

In some embodiments, the method produces effects equivalent to the sum of the effects of each of the agent which interacts with a chemokine receptor or VLA-4 inhibitor when administered independently. In other embodiments, the method produces a synergistic effect relative to the effects of each of the agent which interacts with a chemokine receptor or VLA-4 inhibitor when administered independently.

In some embodiments, the hematopoietic stem cells or progenitor cells are LSK-SLAM cells.

In some embodiments, the agent which interacts with a chemokine receptor is selected from plerixafor, Groβ, or a derivative of Groβ. In some embodiments, the derivative of Groβ is a truncated version of Groβ. In some embodiments, the truncated version of Groβ is SB-251353.

In some embodiments, the method further comprises administering an inhibitor of integrin α9β1, G-CSF, a derivative of G-CSF, or a combination thereof. In some embodiments, the inhibitor of integrin α9β1 is (N-benzenesulfonyl)-L-prolyl-L-O-(1-pyrrolidinylcarbonyl)tyrosine (BOP).

In some embodiments, the VLA-4 inhibitor is a compound of the formula:

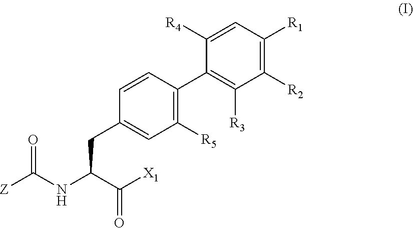

wherein:
R₁ is hydrogen, aminocarbonyl, carboxy, cyano, halo, hydroxy, —SF₅, alkyl_{(C≤8)}, substituted alkyl_{(C≤8)}, alkoxy_{(C≤8)}, substituted alkoxy_{(C≤8)}, or —Y₁—Rₐ; wherein:
   Y₁ is alkanediyl_{(C≤8)} or substituted alkanediyl_{(C≤8)}; and
   Rₐ is alkoxy_{(C≤12)}, acyloxy_{(C≤12)}, or a substituted version of either of these groups; or
—X(CH₂O)ₘ—(CH₂CH₂O)ₙ—R_{b}; wherein:
   X is a covalent bond or —O—;
   m is 0 or 1;
   n is 1, 2, 3, 4, 5, 6, 7, or 8; and
   R_{b} is hydrogen, alkyl_{(C≤8)}, or substituted alkyl_{(C≤8)}; or
—CH₂—O—(CH₂)ₙ—R_{c}; wherein:
   N is 0, 1, or 2; and
      R_{c} is a cyclic aliphatic ring of 4-6 in size having one oxygen as part of the ring;
R₂ is hydrogen, aminocarbonyl, carboxy, cyano, halo, hydroxy, —SF₅, alkyl_{(C≤8)}, substituted alkyl_{(C≤8)}, alkoxy_{(C≤8)}, substituted alkoxy_{(C≤8)}, or —Y₂—R_{c}; wherein:
   Y₂ is alkanediyl_{(C≤8)} or substituted alkanediyl_{(C≤8)}; and
   R_{c} is alkoxy_{(C≤12)}, acyloxy_{(C≤12)}, or a substituted version of either of these groups;
X₁ is hydroxy, alkoxy_{(C≤8)}, substituted alkoxy_{(C≤8)}, cycloalkoxy_{(C≤8)}, substituted cycloalkoxy_{(C≤8)}, alkenyloxy_{(C≤8)}, substituted alkenyloxy_{(C≤8)}, aryloxy_{(C≤8)}, substituted aryloxy_{(C≤8)}, aralkoxy_{(C≤8)}, substituted aralkoxy_{(C≤8)}, or a substituent convertible in vivo to hydroxy; and
R₃ and R₄ are each independently hydrogen, hydroxy, alkoxy_{(C≤8)} or substituted alkoxy_{(C≤8)};
R₅ is hydrogen, —CH(OR_{d})Rₑ, or —C(O)R_{f}, wherein:
   R_{d} is hydrogen, alkyl_{(C≤8)}, or substituted alkyl_{(C≤8)}; and
   Rₑ and R_{f} are each independently alkyl_{(C≤8)} or substituted alkyl_{(C≤8)}; and
Z is a group of the formula:

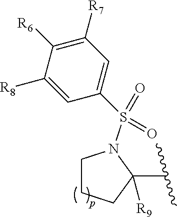

wherein:
   p is 0, 1, 2, or 3;
   R₆ is hydrogen or —C(O)X₂; wherein:
      X₂ is amino, hydroxy, alkoxy_{(C≤8)}, substituted alkoxy_{(C≤8)}, cycloalkoxy_{(C≤8)}, substituted cycloalkoxy_{(C≤8)}, alkenyloxy_{(C≤8)}, substituted alkenyloxy_{(C≤8)}, aryloxy_{(C≤8)}, substituted aryloxy_{(C≤8)}, aralkyloxy_{(C≤8)}, substituted aralkyloxy_{(C≤8)}, alkylamino_{(C≤8)}, substituted alkylamino_{(C≤8)}, dialkylamino_{(C≤8)}, substituted dialkylamino_{(C≤8)}, cycloalkylamino_{(C≤8)}, substituted cycloalkylamino_{(C≤8)}, alkenylamino_{(C≤8)}, substituted alkenylamino_{(C≤8)}, arylamino_{(C≤8)}, substituted arylamino_{(C≤8)}, aralkylamino_{(C≤8)}, substituted aralkylamino_{(C≤8)}, or a substituent convertible in vivo to hydroxy;
   R₇ and R₈ are each independently hydrogen, halo, haloalkyl_{(C≤8)}, or substituted haloalkyl_{(C≤8)};
   R₉ is hydrogen, alkyl_{(C≤8)}, or substituted alkyl_{(C≤8)}; or
a group of the formula:

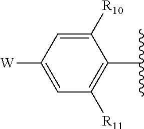

wherein:
   W is hydrogen, cyano, halo, hydroxy, or —C(O)X₃, wherein:
      X₃ is amino, hydroxy, alkoxy_{(C≤8)}, substituted alkoxy_{(C≤8)}, cycloalkoxy_{(C≤8)}, substituted cycloalkoxy_{(C≤8)}, alkenyloxy_{(C≤8)}, substituted alkenyloxy_{(C≤8)}, aryloxy_{(C≤8)}, substituted aryloxy_{(C≤8)}, aralkyloxy_{(C≤8)}, substituted aralkyloxy_{(C≤8)}, alkylamino_{(C≤8)}, substituted alkylamino_{(C≤8)}, dialkylamino_{(C≤8)}, substituted dialkylamino_{(C≤8)}, cycloalkylamino_{(C≤8)}, substituted cycloalkylamino_{(C≤8)}, alkenylamino_{(C≤8)}, substituted alkenylamino_{(C≤8)}, arylamino_{(C≤8)}, substituted arylamino_{(C≤8)}, aralkylamino_{(C≤8)}, substituted aralkylamino_{(C≤8)}, or a substituent convertible in vivo to hydroxy;
   R₁₀ and R₁₁ are each independently hydrogen or halo;

or a pharmaceutically acceptable salt thereof.

In some embodiments the VLA-4 inhibitor is a compound further defined as:

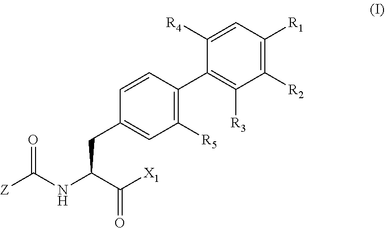

wherein:
R₁ is hydrogen, aminocarbonyl, carboxy, cyano, halo, hydroxy, —SF₅, alkyl_{(C≤8)}, substituted alkyl_{(C≤8)}, alkoxy_{(C≤8)}, substituted alkoxy_{(C≤8)}, or —Y₁—Rₐ; wherein:
   Y₁ is alkanediyl_{(C≤8)} or substituted alkanediyl_{(C≤8)}; and
   Rₐ is alkoxy_{(C≤12)}, acyloxy_{(C≤12)}, or a substituted version of either of these groups; or
—X(CH₂O)ₘ—(CH₂CH₂O)ₙ—R_{b}; wherein:
   X is a covalent bond or —O—;
   m is 0 or 1;
   n is 1, 2, 3, 4, 5, 6, 7, or 8; and
   R_{b} is hydrogen, alkyl_{(C≤8)}, or substituted alkyl_{(C≤8)}; or
—CH₂—O—(CH₂)ₙ—R_{c}; wherein:
   N is 0, 1, or 2; and
   R_{c} is a cyclic aliphatic ring of 4-6 in size having one oxygen as part of the ring;
R₂ is hydrogen, aminocarbonyl, carboxy, cyano, halo, hydroxy, —SF₅, alkyl_{(C≤8)}, substituted alkyl_{(C≤8)}, alkoxy_{(C≤8)}, substituted alkoxy_{(C≤8)}, or —Y₂—R_{c}; wherein:
   Y₂ is alkanediyl_{(C≤8)} or substituted alkanediyl_{(C≤8)}; and
   R_{c} is alkoxy_{(C≤12)}, acyloxy_{(C≤12)}, or a substituted version of either of these groups;
X₁ is hydroxy, alkoxy_{(C≤8)}, substituted alkoxy_{(C≤8)}, cycloalkoxy_{(C≤8)}, substituted cycloalkoxy_{(C≤8)}, alkenyloxy_{(C≤8)}, substituted alkenyloxy_{(C≤8)}, aryloxy_{(C≤8)}, substituted aryloxy_{(C≤8)}, aralkoxy_{(C≤8)}, substituted aralkoxy_{(C≤8)}, or a substituent convertible in vivo to hydroxy; and
R₃ and R₄ are each independently hydrogen, hydroxy, alkoxy_{(C≤8)} or substituted alkoxy_{(C≤8)};
R₅ is hydrogen, —CH(OR_{d})Rₑ, or —C(O)R_{f}, wherein:
   R_{d} is hydrogen, alkyl_{(C≤8)}, substituted alkyl_{(C≤8)}, acyl_{(C≤8)}, or substituted acyl_{(C≤8)}; and
   Rₑ and R_{f} are each independently alkyl_{(C≤8)} or substituted alkyl_{(C≤8)}; and
Z is a group of the formula:

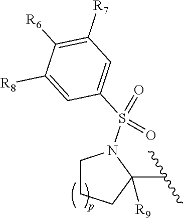

wherein:
   p is 0, 1, 2, or 3;
   R₆ is hydrogen or —C(O)X₂; wherein:
      X₂ is amino, hydroxy, alkoxy_{(C≤8)}, substituted alkoxy_{(C≤8)}, cycloalkoxy_{(C≤8)}, substituted cycloalkoxy_{(C≤8)}, alkenyloxy_{(C≤8)}, substituted alkenyloxy_{(C≤8)}, aryloxy_{(C≤8)}, substituted aryloxy_{(C≤8)}, aralkyloxy_{(C≤8)}, substituted aralkyloxy_{(C≤8)}, alkylamino_{(C≤8)}, substituted alkylamino_{(C≤8)}, dialkylamino_{(C≤8)}, substituted dialkylamino_{(C≤8)}, cycloalkyl-amino_{(C≤8)}, substituted cycloalkylamino_{(C≤8)}, alkenylamino_{(C≤8)}, substituted alkenylamino_{(C≤8)}, arylamino_{(C≤8)}, substituted arylamino_{(C≤8)}, aralkylamino_{(C≤8)}, substituted aralkylamino_{(C≤8)}, or a substituent convertible in vivo to hydroxy;
   R₇ and R₈ are each independently hydrogen, halo, haloalkyl_{(C≤8)}, or substituted haloalkyl_{(C≤8)};
   R₉ is hydrogen, alkyl_{(C≤8)}, or substituted alkyl_{(C≤8)}; or
a group of the formula:

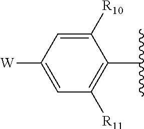

wherein:
   W is hydrogen, cyano, halo, hydroxy, or —C(O)X₃, wherein:
      X₃ is amino, hydroxy, alkoxy_{(C≤8)}, substituted alkoxy_{(C≤8)}, cycloalkoxy_{(C≤8)}, substituted cycloalkoxy_{(C≤8)}, alkenyloxy_{(C≤8)}, substituted alkenyloxy_{(C≤8)}, aryloxy_{(C≤8)}, substituted aryloxy_{(C≤8)}, aralkyloxy_{(C≤8)}, substituted aralkyloxy_{(C≤8)}, alkylamino_{(C≤8)}, substituted alkylamino_{(C≤8)}, dialkylamino_{(C≤8)}, substituted dialkylamino_{(C≤8)}, cycloalkyl-amino_{(C≤8)}, substituted cycloalkylamino_{(C≤8)}, alkenylamino_{(C≤8)}, substituted alkenylamino_{(C≤8)}, arylamino_{(C≤8)}, substituted arylamino_{(C≤8)}, aralkylamino_{(C≤8)}, substituted aralkylamino_{(C≤8)}, or a substituent convertible in vivo to hydroxy;
R₁₀ and R₁₁ are each independently hydrogen or halo. provided that if R₅ is hydrogen, then W is —C(O)X₃; and if W is —C(O)X₃ or R₆ is
—C(O)X₂, then R₉ is hydrogen; and if R₁ is hydrogen, then R₆ is not hydrogen;
   or a pharmaceutically acceptable salt thereof.

In some embodiments, the compounds are further defined by the formula:

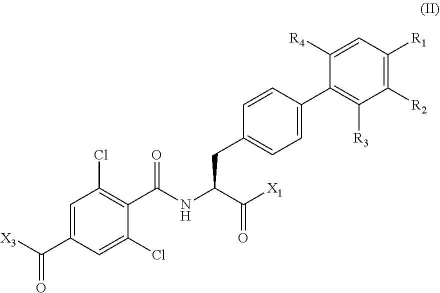

wherein:
R₁ is hydrogen, aminocarbonyl, carboxy, cyano, halo, hydroxy, —SF₅, alkyl_{(C≤8)}, substituted alkyl_{(C≤8)}, alkoxy_{(C≤8)}, substituted alkoxy_{(C≤8)}, or —Y₁—Rₐ; wherein:
   Y₁ is alkanediyl_{(C≤8)} or substituted alkanediyl_{(C≤8)}; and
   Rₐ is alkoxy_{(C≤12)}, acyloxy_{(C≤12)}, or a substituted version of either of these groups; or
—X(CH₂O)ₘ—(CH₂CH₂O)ₙ—R_{b}; wherein:
   X is a covalent bond or —O—;
   m is 0 or 1;
   n is 1, 2, 3, 4, 5, 6, 7, or 8; and
   R_{b} is hydrogen, alkyl_{(C≤8)}, or substituted alkyl_{(C≤8)}; or
—CH₂—O—(CH₂)ₙ—R_{c}; wherein:
   N is 0, 1, or 2; and
   R_{c} is a cyclic aliphatic ring of 4-6 in size having one oxygen as part of the ring;
R₂ is hydrogen, aminocarbonyl, carboxy, cyano, halo, hydroxy, —SF₅, alkyl_{(C≤8)}, substituted alkyl_{(C≤8)}, alkoxy_{(C≤8)}, substituted alkoxy_{(C≤8)}, or —Y₂—R_{c}; wherein:
   Y₂ is alkanediyl_{(C≤8)} or substituted alkanediyl_{(C≤8)}; and
   R_{c} is alkoxy_{(C≤12)}, acyloxy_{(C≤12)}, or a substituted version of either of these groups;
X₁ and X₃ are each independently hydroxy, alkoxy_{(C≤8)}, substituted alkoxy_{(C≤8)}, cycloalkoxy_{(C≤8)}, substituted cycloalkoxy_{(C≤8)}, alkenyloxy_{(C≤8)}, substituted alkenyloxy_{(C≤8)}, aryloxy_{(C≤8)}, substituted aryloxy_{(C≤8)}, aralkoxy_{(C≤8)}, substituted aralkoxy_{(C≤8)}, or a substituent convertible in vivo to hydroxy; and
R₃ and R₄ are each independently alkoxy_{(C≤8)} or substituted alkoxy_{(C≤8)};
provided that R₁ and R₂ are not both hydrogen;
   or a pharmaceutically acceptable salt thereof.

In some embodiments, the compounds are further defined as:

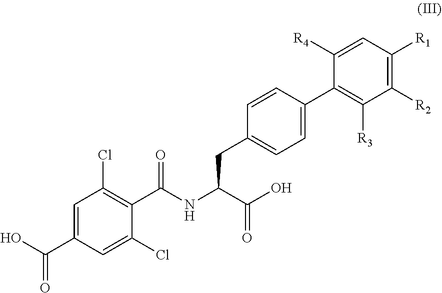

wherein:
R₁ is hydrogen, aminocarbonyl, carboxy, cyano, halo, hydroxy, —SF₅, alkyl_{(C≤8)}, substituted alkyl_{(C≤8)}, alkoxy_{(C≤8)}, substituted alkoxy_{(C≤8)}, or —Y₁—Rₐ; wherein:
   Y₁ is alkanediyl_{(C≤8)} or substituted alkanediyl_{(C≤8)}; and
   Rₐ is alkoxy_{(C≤12)}, acyloxy_{(C≤12)}, or a substituted version of either of these groups; or
—X(CH₂O)ₘ—(CH₂CH₂O)ₙ—R_{b}; wherein:
   X is a covalent bond or —O—;
   m is 0 or 1;
   n is 1, 2, 3, 4, 5, 6, 7, or 8; and
   R_{b} is hydrogen, alkyl_{(C≤8)}, or substituted alkyl_{(C≤8)}; or
—CH₂—O—(CH₂)ₙ—R_{c}; wherein:
   N is 0, 1, or 2; and
   R_{c} is a cyclic aliphatic ring of 4-6 in size having one oxygen as part of the ring;
R₂ is hydrogen, aminocarbonyl, carboxy, cyano, halo, hydroxy, —SF₅, alkyl_{(C≤8)}, substituted alkyl_{(C≤8)}, alkoxy_{(C≤8)}, substituted alkoxy_{(C≤8)}, or —Y₂—R_{c}; wherein:
   Y₂ is alkanediyl_{(C≤8)} or substituted alkanediyl_{(C≤8)}; and
   R_{c} is alkoxy_{(C≤12)}, acyloxy_{(C≤12)}, or a substituted version of either of these groups; and
R₃ and R₄ are each independently alkoxy_{(C≤8)} or substituted alkoxy_{(C≤8)};
provided that R₁ and R₂ are not both hydrogen;
   or a pharmaceutically acceptable salt thereof.

In some embodiments, the compounds are further defined as:

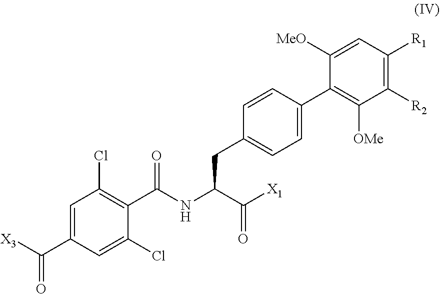

wherein:
R₁ is aminocarbonyl, carboxy, cyano, halo, hydroxy, —SF₅, alkyl_{(C≤8)}, substituted alkyl_{(C≤8)}, alkoxy_{(c≤8)}, substituted alkoxy_{(c≤8)}, or —Y₁—Rₐ; wherein:
   Y₁ is alkanediyl_{(C≤8)} or substituted alkanediyl_{(C≤8)}; and
   Rₐ is alkoxy_{(C≤12)}, acyloxy_{(C≤12)}, or a substituted version of either of these groups; or
—X(CH₂O)ₘ—(CH₂CH₂O)ₙ—R_{b}; wherein:
   X is a covalent bond or —O—;
   m is 0 or 1;
   n is 1, 2, 3, 4, 5, 6, 7, or 8; and
   R_{b} is hydrogen, alkyl_{(C≤8)}, or substituted alkyl_{(C≤8)}; or
—CH₂—O—(CH₂)ₙ—R_{c}; wherein:
   N is 0, 1, or 2; and
   R_{c} is a cyclic aliphatic ring of 4-6 in size having one oxygen as part of the ring;
R₂ is hydrogen, aminocarbonyl, carboxy, cyano, halo, hydroxy, —SF₅, alkyl_{(C≤8)}, substituted alkyl_{(C≤8)}, alkoxy_{(C≤8)}, substituted alkoxy_{(C≤8)}, or —Y₂—R_{c}; wherein:
   Y₂ is alkanediyl_{(C≤8)} or substituted alkanediyl_{(C≤8)}; and
   R_{c} is alkoxy_{(C≤12)}, acyloxy_{(C≤12)}, or a substituted version of either of these groups;
X₁ and X₃ are each independently hydroxy, alkoxy_{(C≤8)}, substituted alkoxy_{(C≤8)}, cycloalkoxy_{(C≤8)}, substituted cycloalkoxy_{(C≤8)}, alkenyloxy_{(C≤8)}, substituted alkenyloxy_{(C≤8)}, aryloxy_{(C≤8)}, substituted aryloxy_{(C≤8)}, aralkoxy_{(C≤8)}, substituted aralkoxy_{(C≤8)}, or a group convertible in vivo to hydroxy;
   or a pharmaceutically acceptable salt thereof.

In some embodiments, the compounds are further defined as:

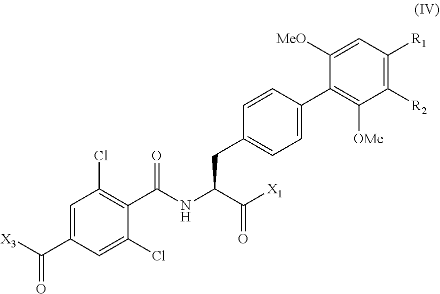

wherein:
R₁ is hydrogen, aminocarbonyl, carboxy, cyano, halo, hydroxy, —SF₅, alkyl_{(C≤8)}, substituted alkyl_{(C≤8)}, alkoxy_{(C≤8)}, substituted alkoxy_{(C≤8)}, or —Y₁—Rₐ; wherein:
   Y₁ is alkanediyl_{(C≤8)} or substituted alkanediyl_{(C≤8)}; and
   Rₐ is alkoxy_{(C≤12)}, acyloxy_{(C≤12)}, or a substituted version of either of these groups; or
—X(CH₂O)ₘ—(CH₂CH₂O)ₙ—R_{b}; wherein:
   X is a covalent bond or —O—;
   m is 0 or 1;
   n is 1, 2, 3, 4, 5, 6, 7, or 8; and
   R_{b} is hydrogen, alkyl_{(C≤8)}, or substituted alkyl_{(C≤8)}; or
—CH₂—O—(CH₂)ₙ—R_{c}; wherein:
   N is 0, 1, or 2; and
   R_{c} is a cyclic aliphatic ring of 4-6 in size having one oxygen as part of the ring;
R₂ is aminocarbonyl, carboxy, cyano, halo, hydroxy, —SF₅, alkyl_{(C≤8)}, substituted alkyl_{(C≤8)}, alkoxy_{(C≤8)}, substituted alkoxy_{(C≤8)}, or —Y₂—R_{c}; wherein:
   Y₂ is alkanediyl_{(C≤8)} or substituted alkanediyl_{(C≤8)}; and
   R_{c} is alkoxy_{(C≤12)}, acyloxy_{(C≤12)}, or a substituted version of either of these groups;
X₁ and X₃ are each independently hydroxy, alkoxy_{(C≤8)}, substituted alkoxy_{(C≤8)}, cycloalkoxy_{(C≤8)}, substituted cycloalkoxy_{(C≤8)}, alkenyloxy_{(C≤8)}, substituted alkenyloxy_{(C≤8)}, aryloxy_{(C≤8)}, substituted aryloxy_{(C≤8)}, aralkoxy_{(C≤8)}, substituted aralkoxy_{(C≤8)}, or a group convertible in vivo to hydroxy;
   or a pharmaceutically acceptable salt thereof.

In some embodiments, the compounds are further defined as:

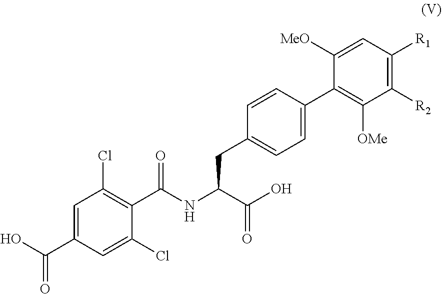

wherein:
R₁ is hydrogen, aminocarbonyl, carboxy, cyano, halo, hydroxy, —SF₅, alkyl_{(C≤8)}, substituted alkyl_{(C≤8)}, alkoxy_{(C≤8)}, substituted alkoxy_{(C≤8)}, or —Y₁—Rₐ; wherein:
   Y₁ is alkanediyl_{(C≤8)} or substituted alkanediyl_{(C≤8)}; and
   Rₐ is alkoxy_{(C≤12)}, acyloxy_{(C≤12)}, or a substituted version of either of these groups; or
—X(CH₂O)ₘ—(CH₂CH₂O)ₙ—R_{b}; wherein:
   X is a covalent bond or —O—;
   m is 0 or 1;
   n is 1, 2, 3, 4, 5, 6, 7, or 8; and
   R_{b} is hydrogen, alkyl_{(C≤8)}, or substituted alkyl_{(C≤8)}; or
—CH₂—O—(CH₂)ₙ—R_{c}; wherein:
   N is 0, 1, or 2; and
   R_{c} is a cyclic aliphatic ring of 4-6 in size having one oxygen as part of the ring;
R₂ is hydrogen, aminocarbonyl, carboxy, cyano, halo, hydroxy, —SF₅, alkyl_{(C≤8)}, substituted alkyl_{(C≤8)}, alkoxy_{(C≤8)}, substituted alkoxy_{(C≤8)}, or —Y₂—R_{c}; wherein:
   Y₂ is alkanediyl_{(C≤8)} or substituted alkanediyl_{(C≤8)}; and
   R_{c} is alkoxy_{(C≤12)}, acyloxy_{(C≤12)}, or a substituted version of either of these groups;
provided that R₁ and R₂ are not both hydrogen;
   or a pharmaceutically acceptable salt thereof.

In some embodiments, the compounds are further defined as:

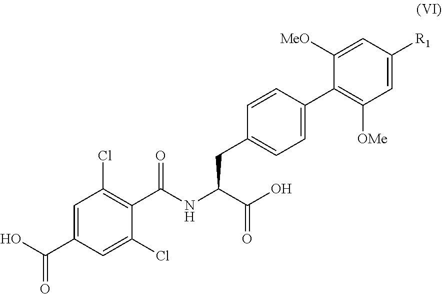

wherein:
R₁ is alkyl_{(C≤8)}, substituted alkyl_{(C≤8)}, alkoxy_{(C≤8)}, substituted alkoxy_{(C≤8)}, or —Y₁—Rₐ; wherein:
   Y₁ is alkanediyl_{(C≤8)} or substituted alkanediyl_{(C≤8)}; and
   Rₐ is alkoxy_{(C≤12)}, acyloxy_{(C≤12)}, or a substituted version of either of these groups; or
—X(CH₂O)ₘ—(CH₂CH₂O)ₙ—R_{b}; wherein:
   X is a covalent bond or —O—;
   m is 0 or 1;
   n is 1, 2, 3, 4, 5, 6, 7, or 8; and
   R_{b} is hydrogen, alkyl_{(C≤8)}, or substituted alkyl_{(C≤8)}; or
—CH₂—O—(CH₂)ₙ—R_{c}; wherein:
   N is 0, 1, or 2; and
   R_{c} is a cyclic aliphatic ring of 4-6 in size having one oxygen as part of the ring;
      or a pharmaceutically acceptable salt thereof.

In other embodiments, the compounds are further defined as:

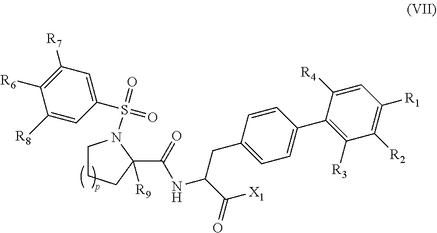

wherein:
R₁ is hydrogen, aminocarbonyl, carboxy, cyano, halo, hydroxy, —SF₅, alkyl_{(C≤8)}, substituted alkyl_{(C≤8)}, alkoxy_{(C≤8)}, substituted alkoxy_{(C≤8)}, or —Y₁—Rₐ, wherein:
   Y₁ is alkanediyl_{(C≤8)} or substituted alkanediyl_{(C≤8)}; and
   Rₐ is alkoxy_{(C≤12)}, acyloxy_{(C≤12)}, or a substituted version of either group; or
—X(CH₂O)ₘ—(CH₂CH₂O)ₙ—R_{b}, wherein:
   X is a covalent bond or —O—;
   m is 0 or 1;
   n is 1, 2, 3, 4, 5, 6, 7, or 8; and
   R_{b} is hydrogen, alkyl_{(C≤8)}, or substituted alkyl_{(C≤8)}; or
—CH₂—O—(CH₂)ₙ—R_{c}; wherein:
   N is 0, 1, or 2; and
   R_{c} is a cyclic aliphatic ring of 4-6 in size having one oxygen as part of the ring;
R₂ is hydrogen, aminocarbonyl, carboxy, cyano, halo, hydroxy, —SF₅, alkyl_{(C≤8)}, substituted alkyl_{(C≤8)}, alkoxy_{(C≤8)}, substituted alkoxy_{(C≤8)}, or —Y₂—R_{c}, wherein:
   Y₂ is alkanediyl_{(C≤8)} or substituted alkanediyl_{(C≤8)}; and
   R_{c} is alkoxy_{(C≤12)}, acyloxy_{(C≤12)}, or a substituted version of either group; or
R₃ and R₄ are each independently alkoxy_{(C≤8)} or substituted alkoxy_{(C≤8)};
R₇ and R₈ are each independently hydrogen, halo, haloalkyl_{(C≤8)}, or substituted haloalkyl_{(C≤8)};
R₆ is hydrogen or —C(O)X₂;
R₉ is hydrogen, alkyl_{(C≤6)}, or substituted alkyl_{(C≤6)};
p is 0, 1, 2, or 3; and
X₁ and X₂ are each independently hydroxy, alkoxy_{(C≤8)}, substituted alkoxy_{(C≤8)}, cycloalkoxy_{(C≤8)}, substituted cycloalkoxy_{(C≤8)}, alkenyloxy_{(C≤8)}, substituted alkenyloxy_{(C≤8)}, aryloxy_{(C≤8)}, substituted aryloxy_{(C≤8)}, aralkyloxy_{(C≤8)}, substituted aralkyloxy_{(C≤8)}, or a substituent convertible in vivo to hydroxy;
provided that R₁ and R₇ are not both hydrogen;
   or a pharmaceutically acceptable salt thereof.

In some embodiments, the compounds are further defined as:

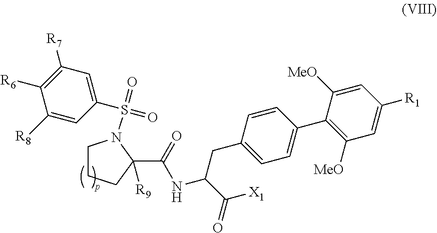

wherein:
R₁ is hydrogen, aminocarbonyl, carboxy, cyano, halo, hydroxy, —SF₅, alkyl_{(C≤8)}, substituted alkyl_{(C≤8)}, alkoxy_{(C≤8)}, substituted alkoxy_{(C≤8)}, or —Y₁—Rₐ, wherein:
   Y₁ is alkanediyl_{(C≤8)} or substituted alkanediyl_{(C≤8)}; and
   Rₐ is alkoxy_{(C≤12)}, acyloxy_{(C≤12)}, or a substituted version of either group; or
—X(CH₂O)ₘ—(CH₂CH₂O)ₙ—R_{b}, wherein:
   X is a covalent bond or —O—;
   m is 0 or 1;
   n is 1, 2, 3, 4, 5, 6, 7, or 8; and
   R_{b} is hydrogen, alkyl_{(C≤8)}, or substituted alkyl_{(C≤8)}; or
—CH₂—O—(CH₂)ₙ—R_{c}; wherein:
   N is 0, 1, or 2; and
   R_{c} is a cyclic aliphatic ring of 4-6 in size having one oxygen as part of the ring;
R₇ and R₈ are each independently hydrogen, halo, haloalkyl_{(C≤8)}, or substituted haloalkyl_{(C≤8)};
R₆ is hydrogen or —C(O)X₂;
R₉ is hydrogen, alkyl_{(C≤6)}, or substituted alkyl_{(C≤6)};
p is 0, 1, 2, or 3; and
X₁ and X₂ are each independently hydroxy, alkoxy_{(C≤8)}, substituted alkoxy_{(C≤8)}, cycloalkoxy_{(C≤8)}, substituted cycloalkoxy_{(C≤8)}, alkenyloxy_{(C≤8)}, substituted alkenyloxy_{(C≤8)}, aryloxy_{(C≤8)}, substituted aryloxy_{(C≤8)}, aralkyloxy_{(C≤8)}, substituted aralkyloxy_{(C≤8)}, or a substituent convertible in vivo to hydroxy;
provided that R₁ and R₇ are not both hydrogen;
   or a pharmaceutically acceptable salt thereof.

In some embodiments, the compounds are further defined as:

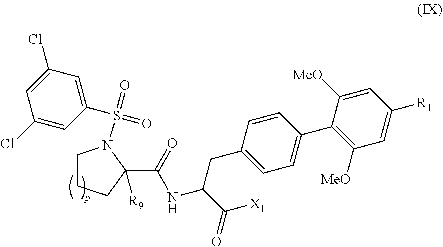

wherein:
R₁ is aminocarbonyl, carboxy, cyano, halo, hydroxy, —SF₅, alkyl_{(C≤8)}, substituted alkyl_{(C≤8)}, alkoxy_{(C≤8)}, substituted alkoxy_{(C≤8)}, or —Y₁—Rₐ, wherein:
   Y₁ is alkanediyl_{(C≤8)} or substituted alkanediyl_{(C≤8)}; and
   Rₐ is alkoxy_{(C≤12)}, acyloxy_{(C≤12)}, or a substituted version of either group; or
—X(CH₂O)ₘ—(CH₂CH₂O)ₙ—R_{b}, wherein:
   X is a covalent bond or —O—;
   m is 0 or 1;
   n is 1, 2, 3, 4, 5, 6, 7, or 8; and
   R_{b} is hydrogen, alkyl_{(C≤8)}, or substituted alkyl_{(C≤8)}; or
—CH₂—O—(CH₂)ₙ—R_{c}; wherein:
   N is 0, 1, or 2; and
   R_{c} is a cyclic aliphatic ring of 4-6 in size having one oxygen as part of the ring;
R₉ is hydrogen, alkyl_{(C≤6)}, or substituted alkyl_{(C≤6)};
p is 0, 1, 2, or 3; and
X₁ is hydroxy, alkoxy_{(C≤8)}, substituted alkoxy_{(C≤8)}, cycloalkoxy_{(C≤8)}, substituted cycloalkoxy_{(C≤8)}, alkenyloxy_{(C≤8)}, substituted alkenyloxy_{(C≤8)}, aryloxy_{(C≤8)}, substituted aryloxy_{(C≤8)}, aralkyloxy_{(C≤8)}, substituted aralkyloxy_{(C≤8)}, or a substituent convertible in vivo to hydroxy;
   or a pharmaceutically acceptable salt thereof.

In some embodiments, the compounds are further defined as:

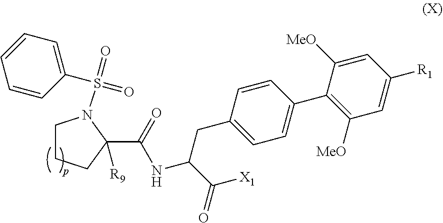

wherein:
R₁ is aminocarbonyl, carboxy, cyano, halo, hydroxy, —SF₅, alkyl_{(C≤8)}, substituted alkyl_{(C≤8)}, alkoxy_{(C≤8)}, substituted alkoxy_{(C≤8)}, or —Y₁—Rₐ, wherein:
   Y₁ is alkanediyl_{(C≤8)} or substituted alkanediyl_{(C≤8)}; and
   Rₐ is alkoxy_{(C≤12)}, acyloxy_{(C≤12)}, or a substituted version of either group; or
—X(CH₂O)ₘ—(CH₂CH₂O)ₙ—R_{b}, wherein:
   X is a covalent bond or —O—;
   m is 0 or 1;
   n is 1, 2, 3, 4, 5, 6, 7, or 8; and
   R_{b} is hydrogen, alkyl_{(C≤8)}, or substituted alkyl_{(C≤8)}; or
—CH₂—O—(CH₂)ₙ—R_{c}; wherein:
   N is 0, 1, or 2; and
   R_{c} is a cyclic aliphatic ring of 4-6 in size having one oxygen as part of the ring;
R₉ is hydrogen, alkyl_{(C≤6)}, or substituted alkyl_{(C≤6)};
p is 0, 1, 2, or 3; and
X₁ is hydroxy, alkoxy_{(C≤8)}, substituted alkoxy_{(C≤8)}, cycloalkoxy_{(C≤8)}, substituted cycloalkoxy_{(C≤8)}, alkenyloxy_{(C≤8)}, substituted alkenyloxy_{(C≤8)}, aryloxy_{(C≤8)}, substituted aryloxy_{(C≤8)}, aralkyloxy_{(C≤8)}, substituted aralkyloxy_{(C≤8)}, or a substituent convertible in vivo to hydroxy;
   or a pharmaceutically acceptable salt thereof.

In some embodiments, the compounds are further defined as:

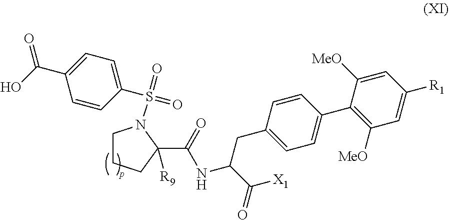

wherein:
R₁ is aminocarbonyl, carboxy, cyano, halo, hydroxy, —SF₅, alkyl_{(C≤8)}, substituted alkyl_{(C≤8)}, alkoxy_{(C≤8)}, substituted alkoxy_{(C≤8)}, or —Y₁—Rₐ, wherein:
   Y₁ is alkanediyl_{(C≤8)} or substituted alkanediyl_{(C≤8)}; and
   Rₐ is alkoxy_{(C≤12)}, acyloxy_{(C≤12)}, or a substituted version of either group; or
—X(CH₂O)ₘ—(CH₂CH₂O)ₙ—R_{b}, wherein:
   X is a covalent bond or —O—;
   m is 0 or 1;
   n is 1, 2, 3, 4, 5, 6, 7, or 8; and
   R_{b} is hydrogen, alkyl_{(C≤8)}, or substituted alkyl_{(C≤8)}; or
—CH₂—O—(CH₂)ₙ—R_{c}; wherein:
   N is 0, 1, or 2; and
   R_{c} is a cyclic aliphatic ring of 4-6 in size having one oxygen as part of the ring;
R₉ is hydrogen, alkyl_{(C≤6)}, or substituted alkyl_{(C≤6)};
p is 0, 1, 2, or 3; and
X₁ is hydroxy, alkoxy_{(C≤8)}, substituted alkoxy_{(C≤8)}, cycloalkoxy_{(C≤8)}, substituted cycloalkoxy_{(C≤8)}, alkenyloxy_{(C≤8)}, substituted alkenyloxy_{(C≤8)}, aryloxy_{(C≤8)}, substituted aryloxy_{(C≤8)}, aralkyloxy_{(C≤8)}, substituted aralkyloxy_{(C≤8)}, or a substituent convertible in vivo to hydroxy;
   or a pharmaceutically acceptable salt thereof.

In other embodiments, the compounds are further defined as:

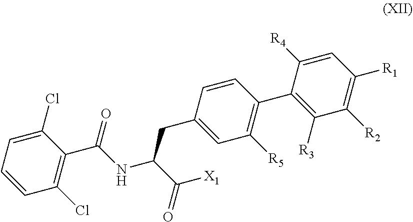

wherein:
R₁ is aminocarbonyl, carboxy, cyano, halo, hydroxy, —SF₅, alkyl_{(C≤8)}, substituted alkyl_{(C≤8)}, alkoxy_{(C≤8)}, substituted alkoxy_{(C≤8)}, or —Y₁—Rₐ, wherein:
   Y₁ is alkanediyl_{(C≤8)} or substituted alkanediyl_{(C≤8)}; and
   Rₐ is alkoxy_{(C≤12)}, acyloxy_{(C≤12)}, or a substituted version of either group; or
—X(CH₂O)ₘ—(CH₂CH₂O)ₙ—R_{b}, wherein:
   X is a covalent bond or —O—;
   m is 0 or 1;
   n is 1, 2, 3, 4, 5, 6, 7, or 8; and
   R_{b} is hydrogen, alkyl_{(C≤8)}, or substituted alkyl_{(C≤8)}; or
—CH₂—O—(CH₂)ₙ—R_{c}; wherein:
   N is 0, 1, or 2; and
   R_{c} is a cyclic aliphatic ring of 4-6 in size having one oxygen as part of the ring;
R₂ is hydrogen, aminocarbonyl, carboxy, cyano, halo, hydroxy, —SF₅, alkyl_{(C≤8)}, substituted alkyl_{(C≤8)}, alkoxy_{(C≤8)}, substituted alkoxy_{(C≤8)}, or —Y₂—R_{c}, wherein:
   Y₂ is alkanediyl_{(C≤8)} or substituted alkanediyl_{(C≤8)}; and
   R_{c} is alkoxy_{(C≤12)}, alkoxy_{(C≤12)}, or a substituted version of either group;
R₃ and R₄ are each independently alkoxy_{(C≤8)} or substituted alkoxy_{(C≤8)};
R₅ is hydrogen, —CH(OR_{d})Rₑ, or —C(O)R_{f}, wherein:
   R_{d} is hydrogen, alkyl_{(C≤8)}, substituted alkyl_{(C≤8)}, acyl_{(C≤8)}, or substituted acyl_{(C≤8)}; and
   Rₑ and R_{f} are each independently alkyl_{(C≤8)} or substituted alkyl_{(C≤8)};
X₁ is hydroxy, alkoxy_{(C≤8)}, substituted alkoxy_{(C≤8)}, cycloalkoxy_{(C≤8)}, substituted cycloalkoxy_{(C≤8)}, alkenyloxy_{(C≤8)}, substituted alkenyloxy_{(C≤8)}, aryloxy_{(C≤8)}, substituted aryloxy_{(C≤8)}, aralkyloxy_{(C≤8)}, substituted aralkyloxy_{(C≤8)}, or a substituent convertible in vivo to hydroxy;
   or a pharmaceutically acceptable salt thereof.

In some embodiments, the compounds are further defined as:

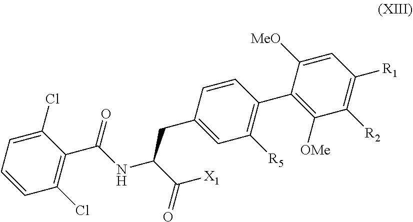

wherein:
R₁ is aminocarbonyl, carboxy, cyano, halo, hydroxy, —SF₅, alkyl_{(C≤8)}, substituted alkyl_{(C≤8)}, alkoxy_{(C≤8)}, substituted alkoxy_{(C≤8)}, or —Y₁—Rₐ, wherein:
   Y₁ is alkanediyl_{(C≤8)} or substituted alkanediyl_{(C≤8)}; and
   Rₐ is alkoxy_{(C≤12)}, acyloxy_{(C≤12)}, or a substituted version of either group; or
—X(CH₂O)ₘ—(CH₂CH₂O)ₙ—R_{b}, wherein:
   X is a covalent bond or —O—;
   m is 0 or 1;
   n is 1, 2, 3, 4, 5, 6, 7, or 8; and
   R_{b} is hydrogen, alkyl_{(C≤8)}, or substituted alkyl_{(C≤8)}; or
—CH₂—O—(CH₂)ₙ—R_{c}; wherein:
   N is 0, 1, or 2; and
   R_{c} is a cyclic aliphatic ring of 4-6 in size having one oxygen as part of the ring;
R₂ is hydrogen, aminocarbonyl, carboxy, cyano, halo, hydroxy, —SF₅, alkyl_{(C≤8)}, substituted alkyl_{(C≤8)}, alkoxy_{(C≤8)}, substituted alkoxy_{(C≤8)}, or —Y₂—R_{c}, wherein:
   Y₂ is alkanediyl_{(C≤8)} or substituted alkanediyl_{(C≤8)}; and
   R_{c} is alkoxy_{(C≤12)}, alkoxy_{(C≤12)}, or a substituted version of either group;
R₅ is hydrogen, —CH(OR_{d})Rₑ, or —C(O)R_{f}, wherein:
   R_{d} is hydrogen, alkyl_{(C≤8)}, substituted alkyl_{(C≤8)}, acyl_{(C≤8)}, or substituted acyl_{(C≤8)}; and
   Rₑ and R_{f} are each independently alkyl_{(C≤8)} or substituted alkyl_{(C≤8)};
X₁ is hydroxy, alkoxy_{(C≤8)}, substituted alkoxy_{(C≤8)}, cycloalkoxy_{(C≤8)}, substituted cycloalkoxy_{(C≤8)}, alkenyloxy_{(C≤8)}, substituted alkenyloxy_{(C≤8)}, aryloxy_{(C≤8)}, substituted aryloxy_{(C≤8)}, aralkyloxy_{(C≤8)}, substituted aralkyloxy_{(C≤8)}, or a substituent convertible in vivo to hydroxy;
   or a pharmaceutically acceptable salt thereof.

In some embodiments, the compounds are further defined as:

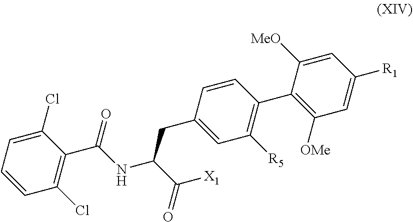

wherein:
R₁ is aminocarbonyl, carboxy, cyano, halo, hydroxy, —SF₅, alkyl_{(C≤8)}, substituted alkyl_{(C≤8)}, alkoxy_{(C≤8)}, substituted alkoxy_{(C≤8)}, or —Y₁—Rₐ, wherein:
   Y₁ is alkanediyl_{(C≤8)} or substituted alkanediyl_{(C≤8)}; and
   Rₐ is alkoxy_{(C≤12)}, acyloxy_{(C≤12)}, or a substituted version of either group; or
—X(CH₂O)ₘ—(CH₂CH₂O)ₙ—R_{b}, wherein:
   X is a covalent bond or —O—;
   m is 0 or 1;
   n is 1, 2, 3, 4, 5, 6, 7, or 8; and
   R_{b} is hydrogen, alkyl_{(C≤8)}, or substituted alkyl_{(C≤8)}; or
—CH₂—O—(CH₂)ₙ—R_{c}; wherein:
   N is 0, 1, or 2; and
   R_{c} is a cyclic aliphatic ring of 4-6 in size having one oxygen as part of the ring;
R₅ is hydrogen, —CH(OR_{d})Rₑ, or —C(O)R_{f}, wherein:
   R_{d} is hydrogen, alkyl_{(C≤8)}, substituted alkyl_{(C≤8)}, acyl_{(C≤8)}, or substituted acyl_{(C≤8)}; and
   Rₑ and R_{f} are each independently alkyl_{(C≤8)} or substituted alkyl_{(C≤8)};
X₁ is hydroxy, alkoxy_{(C≤8)}, substituted alkoxy_{(C≤8)}, cycloalkoxy_{(C≤8)}, substituted cycloalkoxy_{(C≤8)}, alkenyloxy_{(C≤8)}, substituted alkenyloxy_{(C≤8)}, aryloxy_{(C≤8)}, substituted aryloxy_{(C≤8)}, aralkyloxy_{(C≤8)}, substituted aralkyloxy_{(C≤8)}, or a substituent convertible in vivo to hydroxy;
   or a pharmaceutically acceptable salt thereof.

In some embodiments, Z is:

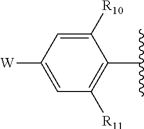

wherein:
W is hydrogen, cyano, halo, hydroxy, or —C(O)X₃, wherein:
   X₃ is amino, hydroxy, alkoxy_{(C≤8)}, substituted alkoxy_{(C≤8)}, cycloalkoxy_{(C≤8)}, substituted cycloalkoxy_{(C≤8)}, alkenyloxy_{(C≤8)}, substituted alkenyloxy_{(C≤8)}, aryloxy_{(C≤8)}, substituted aryloxy_{(C≤8)}, aralkyloxy_{(C≤8)}, substituted aralkyloxy_{(C≤8)}, alkylamino_{(C≤8)}, substituted alkylamino_{(C≤8)}, dialkylamino_{(C≤8)}, substituted dialkylamino_{(C≤8)}, cycloalkylamino_{(C≤8)}, substituted cycloalkylamino_{(C≤8)}, alkenylamino_{(C≤8)}, substituted alkenyl-amino_{(C≤8)}, arylamino_{(C≤8)}, substituted arylamino_{(C≤8)}, aralkylamino_{(C≤8)}, substituted aralkylamino_{(C≤8)}, or a substituent convertible in vivo to hydroxy; and
R₁₀ and R₁₁ are each independently hydrogen or halo.

In some embodiments, Z is:

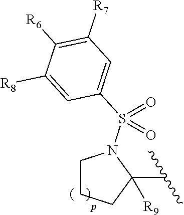

wherein:
p is 0, 1, 2, or 3;
R₆ is hydrogen or —C(O)X₂; wherein:
   X₂ is amino, hydroxy, alkoxy_{(C≤8)}, substituted alkoxy_{(C≤8)}, cycloalkoxy_{(C≤8)}, substituted cycloalkoxy_{(C≤8)}, alkenyloxy_{(C≤8)}, substituted alkenyloxy_{(C≤8)}, aryloxy_{(C≤8)}, substituted aryloxy_{(C≤8)}, aralkyloxy_{(C≤8)}, substituted aralkyloxy_{(C≤8)}, alkylamino_{(C≤8)}, substituted alkylamino_{(C≤8)}, dialkylamino_{(C≤8)}, substituted dialkylamino_{(C≤8)}, cycloalkylamino_{(C≤8)}, substituted cycloalkyl-amino_{(C≤8)}, alkenylamino_{(C≤8)}, substituted alkenylamino_{(C≤8)}, arylamino_{(C≤8)}, substituted arylamino_{(C≤8)}, aralkylamino_{(C≤8)}, substituted aralkylamino_{(C≤8)}, or a substituent convertible in vivo to hydroxy;
R₇ and R₈ are each independently hydrogen, halo, haloalkyl_{(C≤8)}, or substituted haloalkyl_{(C≤8)}; and
R₉ is hydrogen, alkyl_{(C≤8)}, or substituted alkyl_{(C≤8)}.

In some embodiments, R₁ is alkyl_{(C≤8)} or substituted alkyl_{(C≤8)} such as hydroxyalkyl_{(C≤8)} or haloalkyl_{(C≤8)}. In some embodiments, R₁ is an unbranched group. In some embodiments, R₁ is —CH₃, —CH₂OH, —CH(CH₃)OH, —C(CH₃)₂OH, —CH₂F, —CHF₂, —CF₃, —CH₂OCH₃, or —CH₂OCH₂CH₃. In some embodiments, R₁ is alkoxy_{(C≤8)} or substituted alkoxy_{(C≤8)}, such as methoxy or ethoxy. In other embodiments, R₁ is aminocarbonyl or carboxy. In other embodiments, R₁ is aminocarbonyl or carboxy. In other embodiments, R1 is —CH₂—O—(CH2)ₙ—R_{c}; wherein N is 0, 1, or 2; and R_{c} is a cyclic aliphatic ring of 4-6 in size having one oxygen as part of the ring

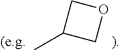

In other embodiments, R₁ is hydroxy, alkyl_{(C≤8)}, substituted alkyl_{(C≤8)}, alkoxy_{(C≤8)}, substituted alkoxy_{(C≤8)}, or —Y₁—Rₐ, wherein:
Y₁ is alkanediyl_{(C≤8)} or substituted alkanediyl_{(C≤8)}; and
Rₐ is alkoxy_{(C≤12)}, acyloxy_{(C≤12)}, or a substituted version of either group; or —X(CH₂O)ₘ—(CH₂CH₂O)ₙ—R_{b}, wherein:
X is a covalent bond or —O—;
m is 0 or 1;
n is 1, 2, 3, 4, 5, 6, 7, or 8; and
R_{b} is hydrogen, alkyl_{(C≤8)}, or substituted alkyl_{(C≤8)}.

In other embodiments, R₁ is —Y₁—Rₐ, wherein: Y₁ is alkanediyl_{(C≤8)} or substituted alkanediyl_{(C≤8)} and Rₐ is alkoxy_{(C≤12)}, acyloxy_{(C≤12)}, or a substituted version of either group. In some embodiments, Y₁ is alkanediyl_{(C≤8)}, such as —CH₂—. In some embodiments, Rₐ is alkoxy_{(C≤12)}, such as ethoxy or hexyloxy. In some embodiments, Rₐ is acyloxy_{(C≤12)}, such as hexanoate. In other embodiments, R₁ is —X(CH₂O)ₘ—(CH₂CH₂O)ₙ—R_{b}, wherein:
X is a covalent bond or —O—;
m is 0 or 1;
n is 1, 2, 3, 4, 5, 6, 7, or 8; and
R_{b} is hydrogen, alkyl_{(C≤8)}, or substituted alkyl_{(C≤8)}.

In some embodiments, X is a covalent bond. In some embodiments, m is 1. In some embodiments, n is 1, 2, or 3. In some embodiments, n is 1. In some embodiments, R_{b} is alkyl_{(C≤8)}, such as methyl. In some embodiments, R₂ is hydrogen. In some embodiments, R₃ is alkoxy_{(C≤6)}, such as methoxy. In some embodiments, R₄ is alkoxy_{(C≤6)}, such as methoxy. In some embodiments, X₁ is hydroxy. In other embodiments, X₁ is a substituent convertible in vivo to hydroxy. In some embodiments, X₂ is hydroxy. In other embodiments, X₂ is a substituent convertible in vivo to hydroxy. In some embodiments, R₇ is hydrogen. In other embodiments, R₇ is halo, such as chloro. In some embodiments, R₈ is hydrogen. In other embodiments, R₈ is halo, such as chloro. In some embodiments, R₆ is hydrogen. In some embodiments, R₉ is hydrogen. In other embodiments, R₉ is alkyl_{(C≤8)}. In some embodiments, R₉ is alkyl_{(C≤4)}, such as methyl.

In some embodiments, p is 0, 1, or 2. In some embodiments, p is 1 or 2. In some embodiments, R₁₀ is halo, such as chloro. In some embodiments, R₁₁ is halo, such as chloro. In some embodiments, W is hydrogen. In other embodiments, W is —C(O)X₃. In other embodiments, X₃ is hydroxy. In other embodiments, X₃ is a substituent convertible in vivo to hydroxy.

In some embodiments, the compound is further defined as:

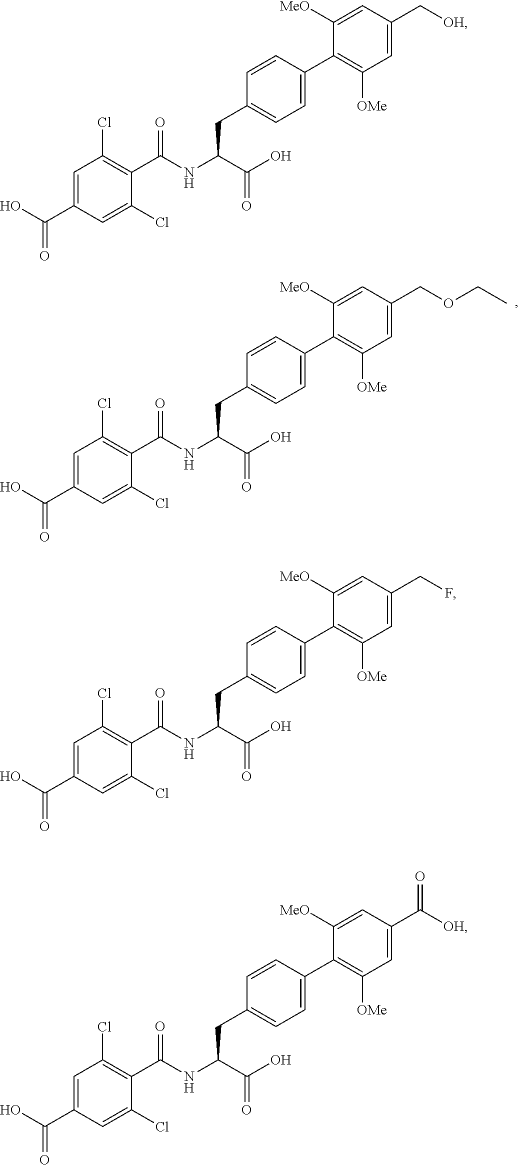 [SMILES: COc1cc(CO)cc(OC)c1-c1ccc(C[C@H](NC(=O)c2c(Cl)cc(C(=O)O)cc2Cl)C(=O)O)cc1 ; CCOCc1cc(OC)c(-c2ccc(C[C@H](NC(=O)c3c(Cl)cc(C(=O)O)cc3Cl)C(=O)O)cc2)c(OC)c1 ; COc1cc(CF)cc(OC)c1-c1ccc(C[C@H](NC(=O)c2c(Cl)cc(C(=O)O)cc2Cl)C(=O)O)cc1 ; COc1cc(C(=O)O)cc(OC)c1-c1ccc(C[C@H](NC(=O)c2c(Cl)cc(C(=O)O)cc2Cl)C(=O)O)cc1]

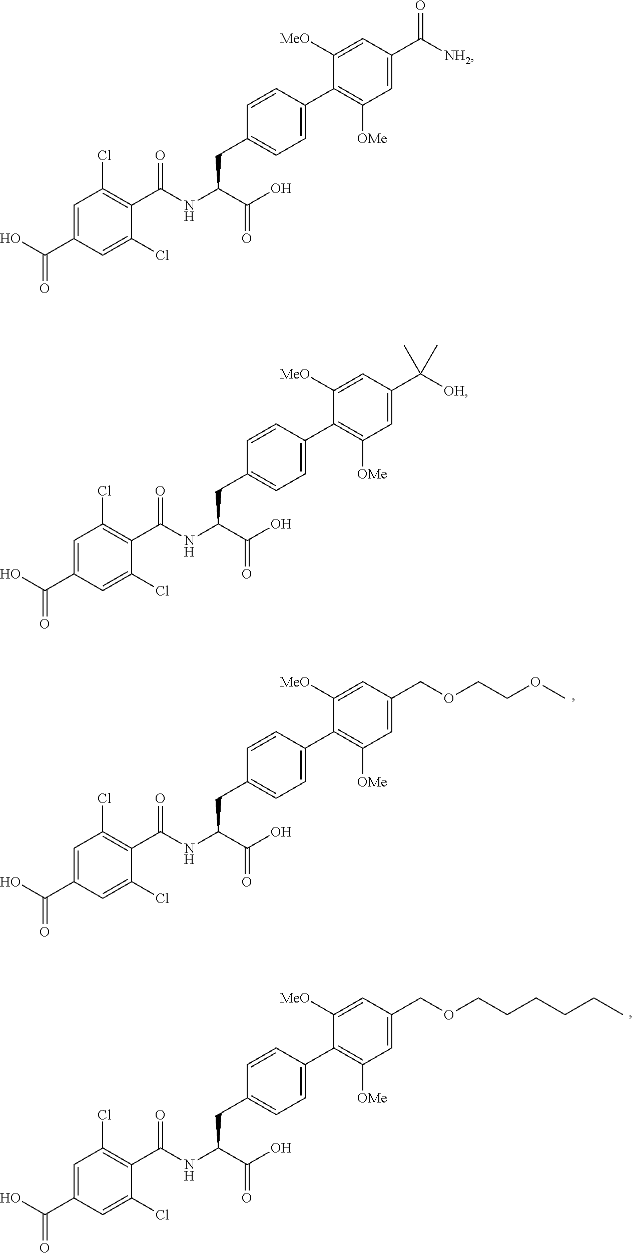 [SMILES: COc1cc(C(N)=O)cc(OC)c1-c1ccc(C[C@H](NC(=O)c2c(Cl)cc(C(=O)O)cc2Cl)C(=O)O)cc1 ; COc1cc(C(C)(C)O)cc(OC)c1-c1ccc(C[C@H](NC(=O)c2c(Cl)cc(C(=O)O)cc2Cl)C(=O)O)cc1 ; COCCOCc1cc(OC)c(-c2ccc(C[C@H](NC(=O)c3c(Cl)cc(C(=O)O)cc3Cl)C(=O)O)cc2)c(OC)c1 ; CCCCCCOCc1cc(OC)c(-c2ccc(C[C@H](NC(=O)c3c(Cl)cc(C(=O)O)cc3Cl)C(=O)O)cc2)c(OC)c1]

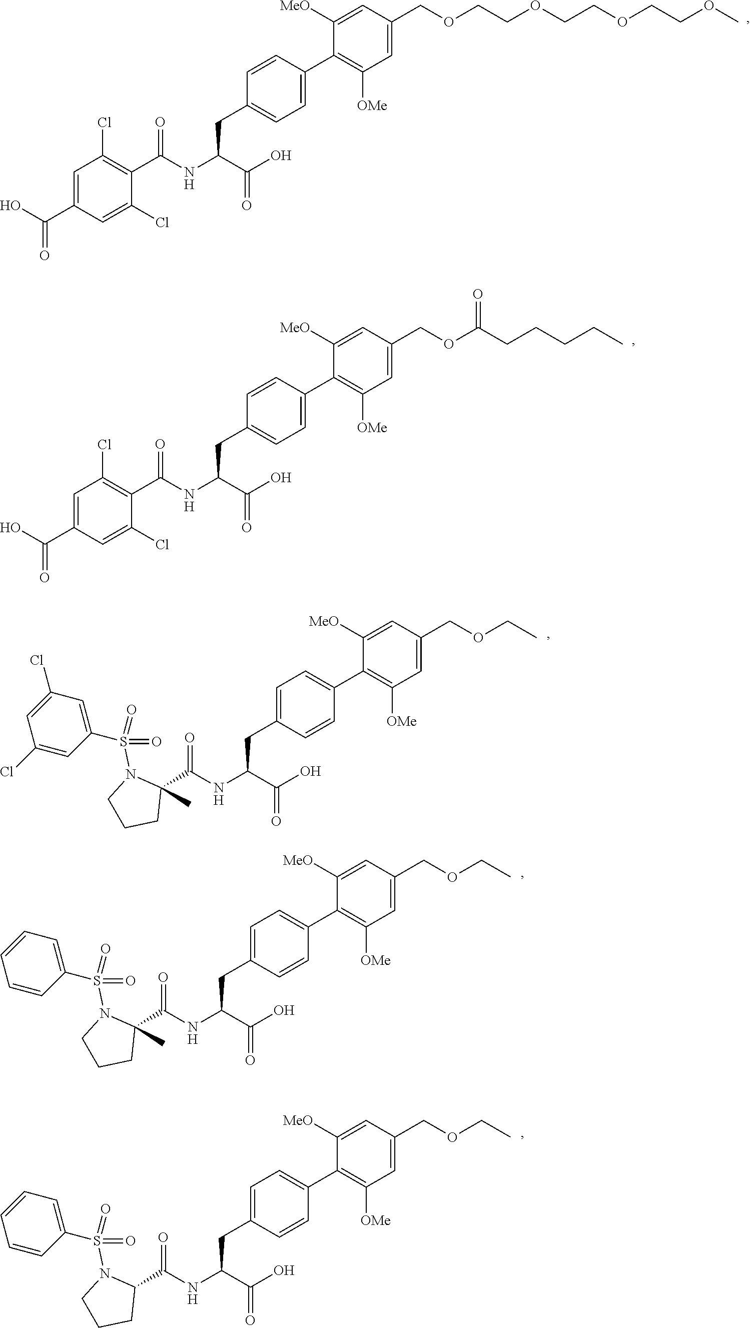 [SMILES: COCCOCCOCCOCc1cc(OC)c(-c2ccc(C[C@H](NC(=O)c3c(Cl)cc(C(=O)O)cc3Cl)C(=O)O)cc2)c(OC)c1 ; CCCCCC(=O)OCc1cc(OC)c(-c2ccc(C[C@H](NC(=O)c3c(Cl)cc(C(=O)O)cc3Cl)C(=O)O)cc2)c(OC)c1 ; CCOCc1cc(OC)c(-c2ccc(C[C@H](NC(=O)[C@]3(C)CCCN3S(=O)(=O)c3cc(Cl)cc(Cl)c3)C(=O)O)cc2)c(OC)c1 ; CCOCc1cc(OC)c(-c2ccc(C[C@H](NC(=O)[C@]3(C)CCCN3S(=O)(=O)c3ccccc3)C(=O)O)cc2)c(OC)c1 ; CCOCc1cc(OC)c(-c2ccc(C[C@H](NC(=O)[C@@H]3CCCN3S(=O)(=O)c3ccccc3)C(=O)O)cc2)c(OC)c1]

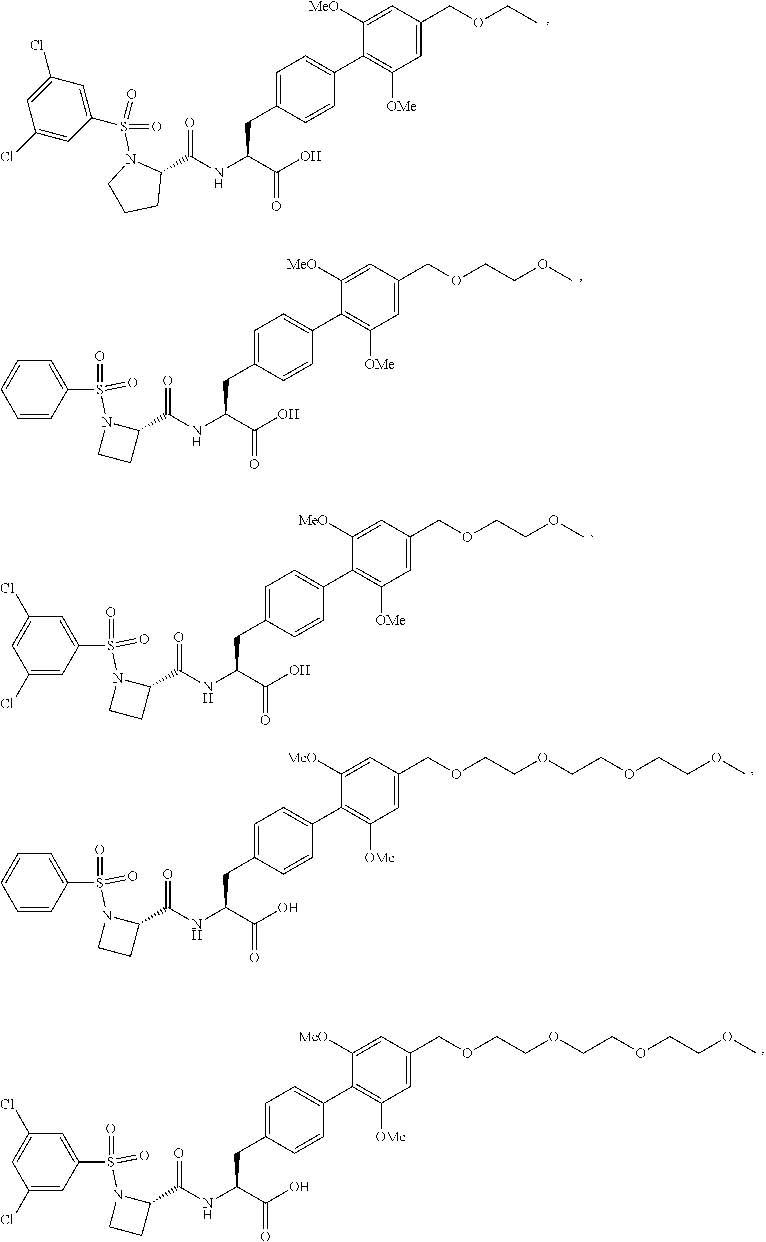 [SMILES: CCOCc1cc(OC)c(-c2ccc(C[C@H](NC(=O)[C@@H]3CCCN3S(=O)(=O)c3cc(Cl)cc(Cl)c3)C(=O)O)cc2)c(OC)c1 ; COCCOCc1cc(OC)c(-c2ccc(C[C@H](NC(=O)[C@@H]3CCN3S(=O)(=O)c3ccccc3)C(=O)O)cc2)c(OC)c1 ; COCCOCc1cc(OC)c(-c2ccc(C[C@H](NC(=O)[C@@H]3CCN3S(=O)(=O)c3cc(Cl)cc(Cl)c3)C(=O)O)cc2)c(OC)c1 ; COCCOCCOCCOCc1cc(OC)c(-c2ccc(C[C@H](NC(=O)[C@@H]3CCN3S(=O)(=O)c3ccccc3)C(=O)O)cc2)c(OC)c1 ; COCCOCCOCCOCc1cc(OC)c(-c2ccc(C[C@H](NC(=O)[C@@H]3CCN3S(=O)(=O)c3cc(Cl)cc(Cl)c3)C(=O)O)cc2)c(OC)c1]

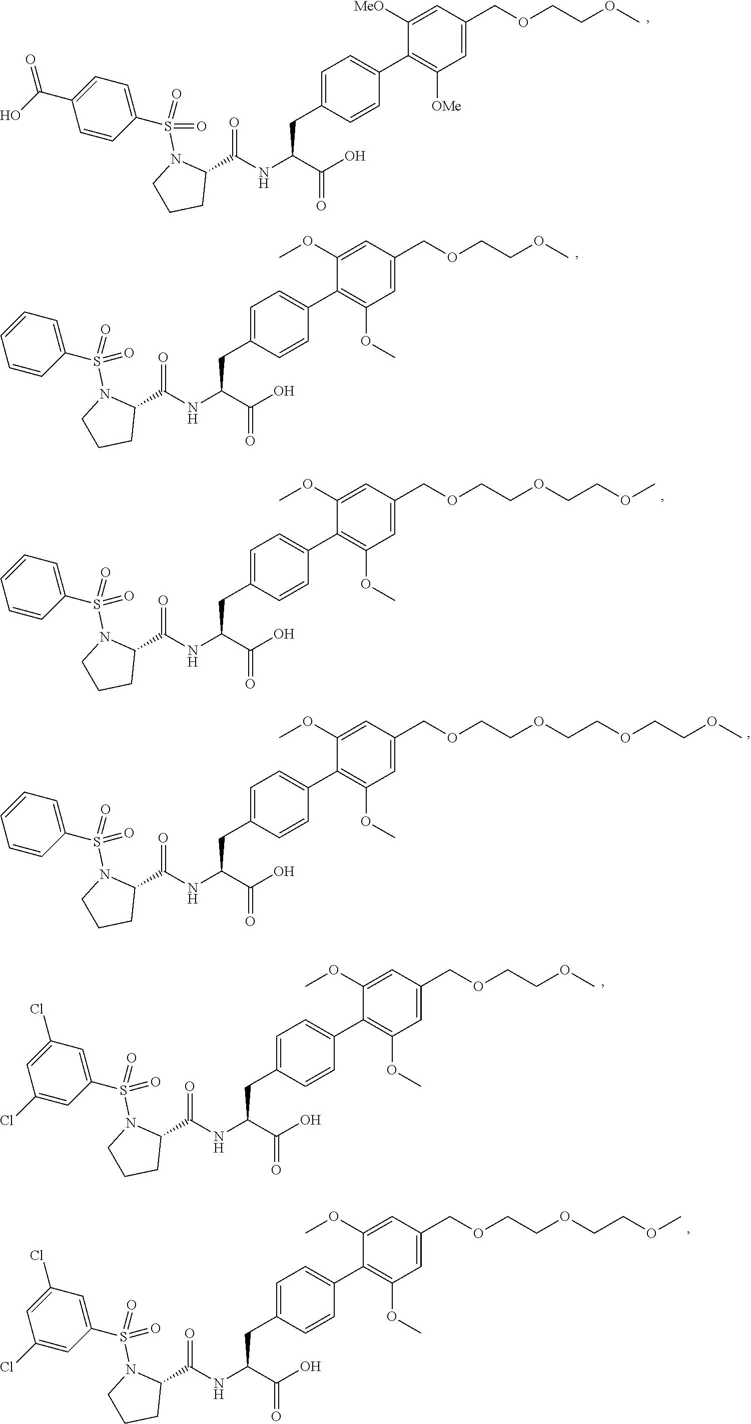

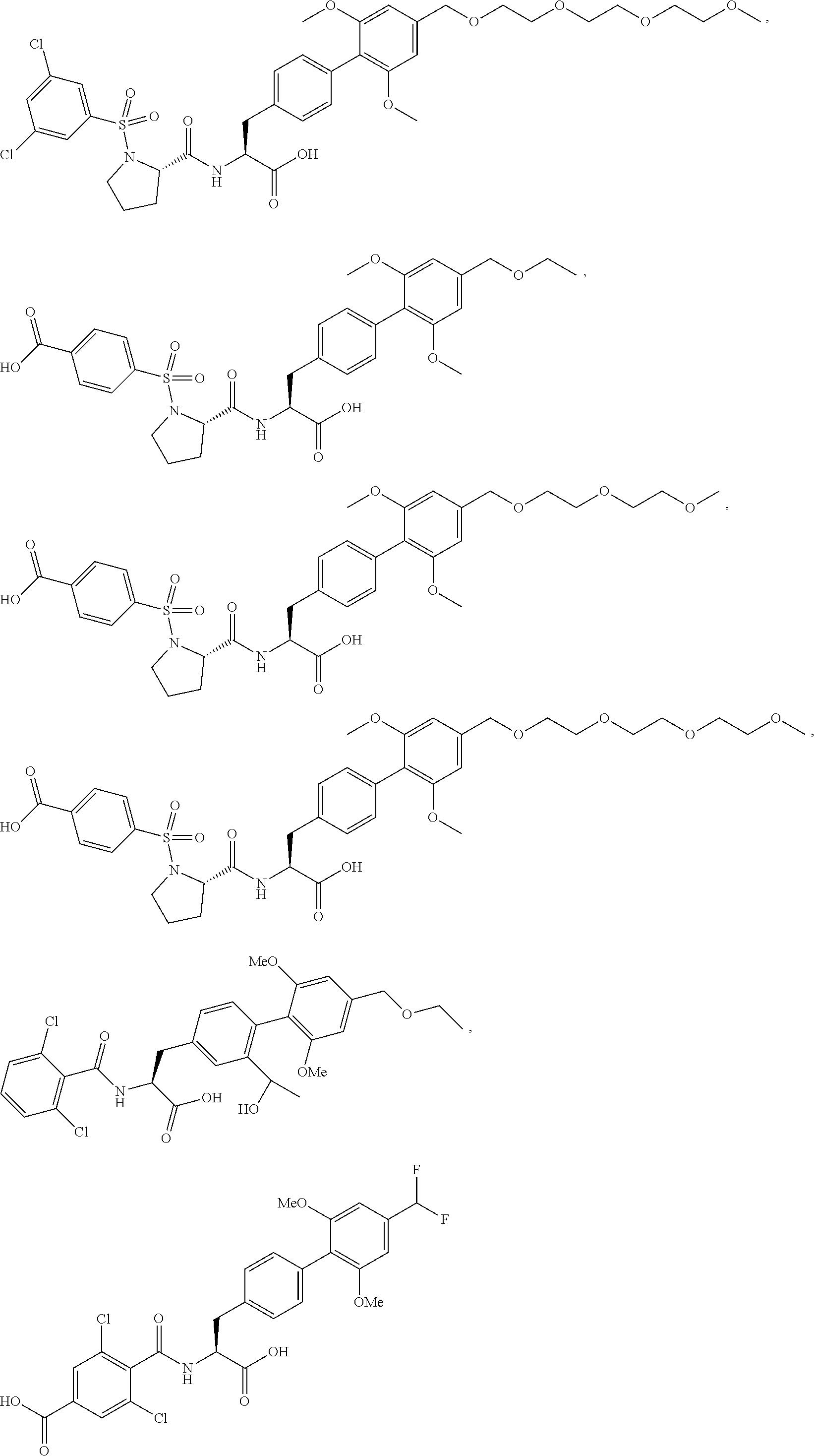

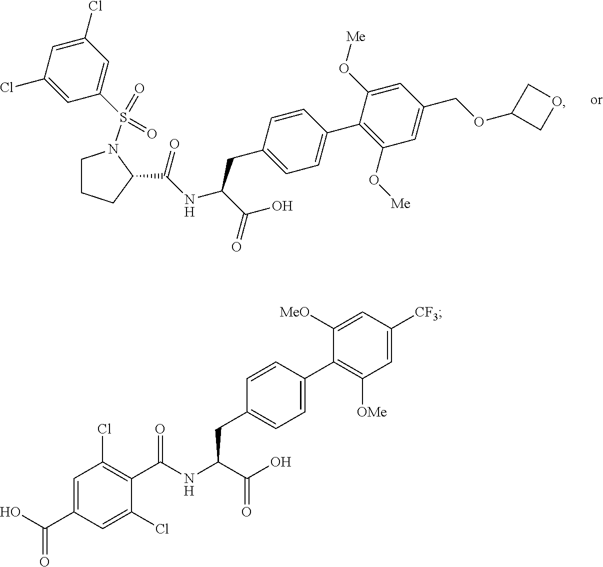 [SMILES: COc1cc(COC2COC2)cc(OC)c1-c1ccc(C[C@H](NC(=O)[C@@H]2CCCN2S(=O)(=O)c2cc(Cl)cc(Cl)c2)C(=O)O)cc1 ; "or" ; COc1cc(C(F)(F)F)cc(OC)c1-c1ccc(C[C@H](NC(=O)c2c(Cl)cc(C(=O)O)cc2Cl)C(=O)O)cc1]

or a pharmaceutically acceptable salt thereof.

In some embodiments, the patient is a mammal such as a human.

In some aspects, the present disclosure provides a composition comprising an agent which interacts with one or more chemokine receptors and a VLA-4 inhibitor. In some embodiments, the VLA-4 inhibitor is a compound of the formula:

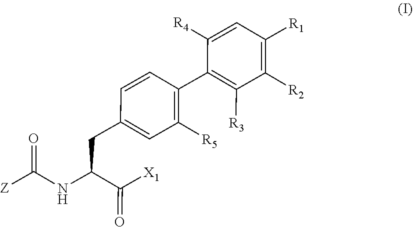

wherein:
R₁ is hydrogen, aminocarbonyl, carboxy, cyano, halo, hydroxy, —SF₅, alkyl_{(C≤8)}, substituted alkyl_{(C≤8)}, alkoxy_{(C≤8)}, substituted alkoxy_{(C≤8)}, or —Y₁—Rₐ; wherein:
   Y₁ is alkanediyl_{(C≤8)} or substituted alkanediyl_{(C≤8)}; and
   Rₐ is alkoxy_{(C≤12)}, acyloxy_{(C≤12)}, or a substituted version of either of these groups; or
—X(CH₂O)ₘ—(CH₂CH₂O)ₙ—R_{b}; wherein:
   X is a covalent bond or —O—;
   m is 0 or 1;
   n is 1, 2, 3, 4, 5, 6, 7, or 8; and
   R_{b} is hydrogen, alkyl_{(C≤8)}, or substituted alkyl_{(C≤8)}; or
—CH₂—O—(CH₂)ₙ—R_{c}; wherein:
   N is 0, 1, or 2; and
   R_{c} is a cyclic aliphatic ring of 4-6 in size having one oxygen as part of the ring;
R₂ is hydrogen, aminocarbonyl, carboxy, cyano, halo, hydroxy, —SF₅, alkyl_{(C≤8)}, substituted alkyl_{(C≤8)}, alkoxy_{(C≤8)}, substituted alkoxy_{(C≤8)}, or —Y₂—R_{c}; wherein:
   Y₂ is alkanediyl_{(C≤8)} or substituted alkanediyl_{(C≤8)}; and
   R_{c} is alkoxy_{(C≤12)}, acyloxy_{(C≤12)}, or a substituted version of either of these groups;
X₁ is hydroxy, alkoxy_{(C≤8)}, substituted alkoxy_{(C≤8)}, cycloalkoxy_{(C≤8)}, substituted cycloalkoxy_{(C≤8)}, alkenyloxy_{(C≤8)}, substituted alkenyloxy_{(C≤8)}, aryloxy_{(C≤8)}, substituted aryloxy_{(C≤8)}, aralkoxy_{(C≤8)}, substituted aralkoxy_{(C≤8)}, or a substituent convertible in vivo to hydroxy; and
R₃ and R₄ are each independently hydrogen, hydroxy, alkoxy_{(C≤8)} or substituted alkoxy_{(C≤8)};
R₅ is hydrogen, —CH(OR_{d})Rₑ, or —C(O)R_{f}, wherein:
   R_{d} is hydrogen, alkyl_{(C≤8)}, or substituted alkyl_{(C≤8)}; and
   Rₑ and R_{f} are each independently alkyl_{(C≤8)} or substituted alkyl_{(C≤8)}; and
Z is a group of the formula:

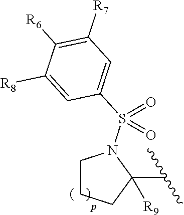

wherein:
   p is 0, 1, 2, or 3;
   R₆ is hydrogen or —C(O)X₂; wherein:
      X₂ is amino, hydroxy, alkoxy_{(C≤8)}, substituted alkoxy_{(C≤8)}, cycloalkoxy_{(C≤8)}, substituted cycloalkoxy_{(C≤8)}, alkenyloxy_{(C≤8)}, substituted alkenyloxy_{(C≤8)}, aryloxy_{(C≤8)}, substituted aryloxy_{(C≤8)}, aralkyloxy_{(C≤8)}, substituted aralkyloxy_{(C≤8)}, alkylamino_{(C≤8)}, substituted alkylamino_{(C≤8)}, dialkylamino_{(C≤8)}, substituted dialkylamino_{(C≤8)}, cycloalkylamino_{(C≤8)}, substituted cycloalkylamino_{(C≤8)}, alkenylamino_{(C≤8)}, substituted alkenylamino_{(C≤8)}, arylamino_{(C≤8)}, substituted arylamino_{(C≤8)}, aralkylamino_{(C≤8)}, substituted aralkylamino_{(C≤8)}, or a substituent convertible in vivo to hydroxy;
   R₇ and R₈ are each independently hydrogen, halo, haloalkyl_{(C≤8)}, or substituted haloalkyl_{(C≤8)};
   R₉ is hydrogen, alkyl_{(C≤8)}, or substituted alkyl_{(C≤8)}; or
a group of the formula:

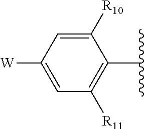

wherein:
   W is hydrogen, cyano, halo, hydroxy, or —C(O)X₃, wherein:
      X₃ is amino, hydroxy, alkoxy_{(C≤8)}, substituted alkoxy_{(C≤8)}, cycloalkoxy_{(C≤8)}, substituted cycloalkoxy_{(C≤8)}, alkenyloxy_{(C≤8)}, substituted alkenyloxy_{(C≤8)}, aryloxy_{(C≤8)}, substituted aryloxy_{(C≤8)}, aralkyloxy_{(C≤8)}, substituted aralkyloxy_{(C≤8)}, alkylamino_{(C≤8)}, substituted alkylamino_{(C≤8)}, dialkylamino_{(C≤8)}, substituted dialkylamino_{(C≤8)}, cycloalkylamino_{(C≤8)}, substituted cycloalkylamino_{(C≤8)}, alkenylamino_{(C≤8)}, substituted alkenylamino_{(C≤8)}, arylamino_{(C≤8)}, substituted arylamino_{(C≤8)}, aralkylamino_{(C≤8)}, substituted aralkylamino_{(C≤8)}, or a substituent convertible in vivo to hydroxy;
   R₁₀ and R₁₁ are each independently hydrogen or halo;
      or a pharmaceutically acceptable salt thereof.

In some embodiments the VLA-4 inhibitor is a compound further defined as:

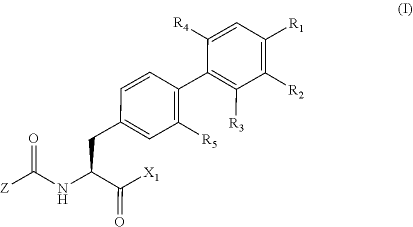

wherein:
R₁ is hydrogen, aminocarbonyl, carboxy, cyano, halo, hydroxy, —SF₅, alkyl_{(C≤8)}, substituted alkyl_{(C≤8)}, alkoxy_{(C≤8)}, substituted alkoxy_{(C≤8)}, or —Y₁—Rₐ; wherein:
   Y₁ is alkanediyl_{(C≤8)} or substituted alkanediyl_{(C≤8)}; and
   Rₐ is alkoxy_{(C≤12)}, acyloxy_{(C≤12)}, or a substituted version of either of these groups; or
—X(CH₂O)ₘ—(CH₂CH₂O)ₙ—R_{b}; wherein:
   X is a covalent bond or —O—;
   m is 0 or 1;
   n is 1, 2, 3, 4, 5, 6, 7, or 8; and
   R_{b} is hydrogen, alkyl_{(C≤8)}, or substituted alkyl_{(C≤8)}; or
—CH₂—O—(CH₂)ₙ—R_{c}; wherein:
   N is 0, 1, or 2; and
   R_{c} is a cyclic aliphatic ring of 4-6 in size having one oxygen as part of the ring;
R₂ is hydrogen, aminocarbonyl, carboxy, cyano, halo, hydroxy, —SF₅, alkyl_{(C≤8)}, substituted alkyl_{(C≤8)}, alkoxy_{(C≤8)}, substituted alkoxy_{(C≤8)}, or —Y₂—R_{c}; wherein:
   Y₂ is alkanediyl_{(C≤8)} or substituted alkanediyl_{(C≤8)}; and
   R_{c} is alkoxy_{(C≤12)}, acyloxy_{(C≤12)}, or a substituted version of either of these groups;
X₁ is hydroxy, alkoxy_{(C≤8)}, substituted alkoxy_{(C≤8)}, cycloalkoxy_{(C≤8)}, substituted cycloalkoxy_{(C≤8)}, alkenyloxy_{(C≤8)}, substituted alkenyloxy_{(C≤8)}, aryloxy_{(C≤8)}, substituted aryloxy_{(C≤8)}, aralkoxy_{(C≤8)}, substituted aralkoxy_{(C≤8)}, or a substituent convertible in vivo to hydroxy; and
R₃ and R₄ are each independently hydrogen, hydroxy, alkoxy_{(C≤8)} or substituted alkoxy_{(C≤8)};
R₅ is hydrogen, —CH(OR_{d})Rₑ, or —C(O)R_{f}, wherein:
   R_{d} is hydrogen, alkyl_{(C≤8)}, substituted alkyl_{(C≤8)}, acyl_{(C≤8)}, or substituted acyl_{(C≤8)}; and
   Rₑ and R_{f} are each independently alkyl_{(C≤8)} or substituted alkyl_{(C≤8)}; and
Z is a group of the formula:

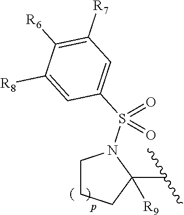

wherein:
   p is 0, 1, 2, or 3;
   R₆ is hydrogen or —C(O)X₂; wherein:
      X₂ is amino, hydroxy, alkoxy_{(C≤8)}, substituted alkoxy_{(C≤8)}, cycloalkoxy_{(C≤8)}, substituted cycloalkoxy_{(C≤8)}, alkenyloxy_{(C≤8)}, substituted alkenyloxy_{(C≤8)}, aryloxy_{(C≤8)}, substituted aryloxy_{(C≤8)}, aralkyloxy_{(C≤8)}, substituted aralkyloxy_{(C≤8)}, alkylamino_{(C≤8)}, substituted alkylamino_{(C≤8)}, dialkylamino_{(C≤8)}, substituted dialkylamino_{(C≤8)}, cycloalkyl-amino_{(C≤8)}, substituted cycloalkylamino_{(C≤8)}, alkenylamino_{(C≤8)}, substituted alkenylamino_{(C≤8)}, arylamino_{(C≤8)}, substituted arylamino_{(C≤8)}, aralkylamino_{(C≤8)}, substituted aralkylamino_{(C≤8)}, or a substituent convertible in vivo to hydroxy;
   R₇ and R₈ are each independently hydrogen, halo, haloalkyl_{(C≤8)}, or substituted haloalkyl_{(C≤8)};
   R₉ is hydrogen, alkyl_{(C≤8)}, or substituted alkyl_{(C≤8)}; or
a group of the formula:

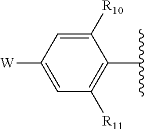

wherein:
   W is hydrogen, cyano, halo, hydroxy, or —C(O)X₃, wherein:
      X₃ is amino, hydroxy, alkoxy_{(C≤8)}, substituted alkoxy_{(C≤8)}, cycloalkoxy_{(C≤8)}, substituted cycloalkoxy_{(C≤8)}, alkenyloxy_{(C≤8)}, substituted alkenyloxy_{(C≤8)}, aryloxy_{(C≤8)}, substituted aryloxy_{(C≤8)}, aralkyloxy_{(C≤8)}, substituted aralkyloxy_{(C≤8)}, alkylamino_{(C≤8)}, substituted alkylamino_{(C≤8)}, dialkylamino_{(C≤8)}, substituted dialkylamino_{(C≤8)}, cycloalkyl-amino_{(C≤8)}, substituted cycloalkylamino_{(C≤8)}, alkenylamino_{(C≤8)}, substituted alkenylamino_{(C≤8)}, arylamino_{(C≤8)}, substituted arylamino_{(C≤8)}, aralkylamino_{(C≤8)}, substituted aralkylamino_{(C≤8)}, or a substituent convertible in vivo to hydroxy;
R₁₀ and R₁₁ are each independently hydrogen or halo. provided that if R₅ is hydrogen, then W is —C(O)X₃; and if W is —C(O)X₃ or R₆ is
—C(O)X₂, then R₉ is hydrogen; and if R₁ is hydrogen, then R₆ is not hydrogen;
   or a pharmaceutically acceptable salt thereof.

In some embodiments, the compounds are further defined by the formula:

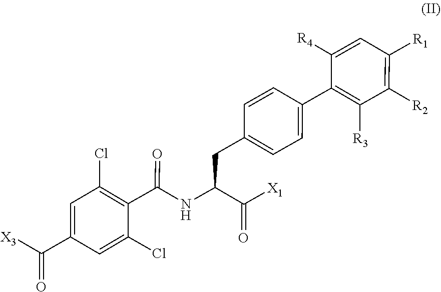

wherein:
R₁ is hydrogen, aminocarbonyl, carboxy, cyano, halo, hydroxy, —SF₅, alkyl_{(C≤8)}, substituted alkyl_{(C≤8)}, alkoxy_{(C≤8)}, substituted alkoxy_{(C≤8)}, or —Y₁—Rₐ; wherein:
   Y₁ is alkanediyl_{(C≤8)} or substituted alkanediyl_{(C≤8)}; and
   Rₐ is alkoxy_{(C≤12)}, acyloxy_{(C≤12)}, or a substituted version of either of these groups; or
—X(CH₂O)ₘ—(CH₂CH₂O)ₙ—R_{b}; wherein:
   X is a covalent bond or —O—;
   m is 0 or 1;
   n is 1, 2, 3, 4, 5, 6, 7, or 8; and
   R_{b} is hydrogen, alkyl_{(C≤8)}, or substituted alkyl_{(C≤8)}; or
—CH₂—O—(CH₂)ₙ—R_{c}; wherein:
   N is 0, 1, or 2; and
   R_{c} is a cyclic aliphatic ring of 4-6 in size having one oxygen as part of the ring;
R₂ is hydrogen, aminocarbonyl, carboxy, cyano, halo, hydroxy, —SF₅, alkyl_{(C≤8)}, substituted alkyl_{(C≤8)}, alkoxy_{(C≤8)}, substituted alkoxy_{(C≤8)}, or —Y₂—R_{c}; wherein:
   Y₂ is alkanediyl_{(C≤8)} or substituted alkanediyl_{(C≤8)}; and
   R_{c} is alkoxy_{(C≤12)}, acyloxy_{(C≤12)}, or a substituted version of either of these groups;
X₁ and X₃ are each independently hydroxy, alkoxy_{(C≤8)}, substituted alkoxy_{(C≤8)}, cycloalkoxy_{(C≤8)}, substituted cycloalkoxy_{(C≤8)}, alkenyloxy_{(C≤8)}, substituted alkenyloxy_{(C≤8)}, aryloxy_{(C≤8)}, substituted aryloxy_{(C≤8)}, aralkoxy_{(C≤8)}, substituted aralkoxy_{(C≤8)}, or a substituent convertible in vivo to hydroxy; and
R₃ and R₄ are each independently alkoxy_{(C≤8)} or substituted alkoxy_{(C≤8)};
provided that R₁ and R₂ are not both hydrogen;
   or a pharmaceutically acceptable salt thereof.

In some embodiments, the compounds are further defined as:

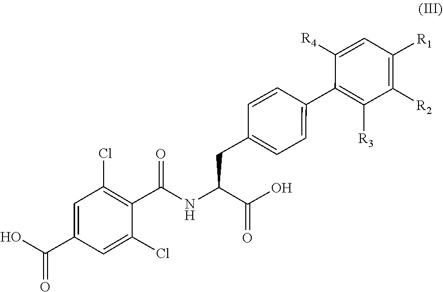

wherein:
R₁ is hydrogen, aminocarbonyl, carboxy, cyano, halo, hydroxy, —SF₅, alkyl_{(C≤8)}, substituted alkyl_{(C≤8)}, alkoxy_{(C≤8)}, substituted alkoxy_{(C≤8)}, or —Y₁—Rₐ; wherein:
   Y₁ is alkanediyl_{(C≤8)} or substituted alkanediyl_{(C≤8)}; and
   Rₐ is alkoxy_{(C≤12)}, acyloxy_{(C≤12)}, or a substituted version of either of these groups; or
—X(CH₂O)ₘ—(CH₂CH₂O)ₙ—R_{b}; wherein:
   X is a covalent bond or —O—;
   m is 0 or 1;
   n is 1, 2, 3, 4, 5, 6, 7, or 8; and
   R_{b} is hydrogen, alkyl_{(C≤8)}, or substituted alkyl_{(C≤8)}; or
—CH₂—O—(CH₂)ₙ—R_{c}; wherein:
   N is 0, 1, or 2; and
   R_{c} is a cyclic aliphatic ring of 4-6 in size having one oxygen as part of the ring;
R₂ is hydrogen, aminocarbonyl, carboxy, cyano, halo, hydroxy, —SF₅, alkyl_{(C≤8)}, substituted alkyl_{(C≤8)}, alkoxy_{(C≤8)}, substituted alkoxy_{(C≤8)}, or —Y₂—R_{c}; wherein:
   Y₂ is alkanediyl_{(C≤8)} or substituted alkanediyl_{(C≤8)}; and
   R_{c} is alkoxy_{(C≤12)}, acyloxy_{(C≤12)}, or a substituted version of either of these groups; and
R₃ and R₄ are each independently alkoxy_{(C≤8)} or substituted alkoxy_{(C≤8)};
provided that R₁ and R₂ are not both hydrogen;
   or a pharmaceutically acceptable salt thereof.

In some embodiments, the compounds are further defined as:

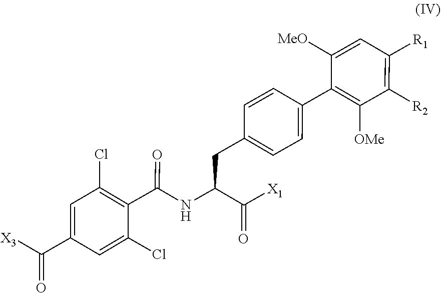

wherein:
R₁ is aminocarbonyl, carboxy, cyano, halo, hydroxy, —SF₅, alkyl_{(C≤8)}, substituted alkyl_{(C≤8)}, alkoxy_{(C≤8)}, substituted alkoxy_{(C≤8)}, or —Y₁—Rₐ; wherein:
   Y₁ is alkanediyl_{(C≤8)} or substituted alkanediyl_{(C≤8)}; and
   Rₐ is alkoxy_{(C≤12)}, acyloxy_{(C≤12)}, or a substituted version of either of these groups; or
—X(CH₂O)ₘ—(CH₂CH₂O)ₙ—R_{b}; wherein:
   X is a covalent bond or —O—;
   m is 0 or 1;
   n is 1, 2, 3, 4, 5, 6, 7, or 8; and
   R_{b} is hydrogen, alkyl_{(C≤8)}, or substituted alkyl_{(C≤8)}; or
—CH₂—O—(CH₂)ₙ—R_{c}; wherein:
   N is 0, 1, or 2; and
   R_{c} is a cyclic aliphatic ring of 4-6 in size having one oxygen as part of the ring;
R₂ is hydrogen, aminocarbonyl, carboxy, cyano, halo, hydroxy, —SF₅, alkyl_{(C≤8)}, substituted alkyl_{(C≤8)}, alkoxy_{(C≤8)}, substituted alkoxy_{(C≤8)}, or —Y₂—R_{c}; wherein:
   Y₂ is alkanediyl_{(C≤8)} or substituted alkanediyl_{(C≤8)}; and
   R_{c} is alkoxy_{(C≤12)}, acyloxy_{(C≤12)}, or a substituted version of either of these groups;
X₁ and X₃ are each independently hydroxy, alkoxy_{(C≤8)}, substituted alkoxy_{(C≤8)}, cycloalkoxy_{(C≤8)}, substituted cycloalkoxy_{(C≤8)}, alkenyloxy_{(C≤8)}, substituted alkenyloxy_{(C≤8)}, aryloxy_{(C≤8)}, substituted aryloxy_{(C≤8)}, aralkoxy_{(C≤8)}, substituted aralkoxy_{(C≤8)}, or a group convertible in vivo to hydroxy;
   or a pharmaceutically acceptable salt thereof.

In some embodiments, the compounds are further defined as:

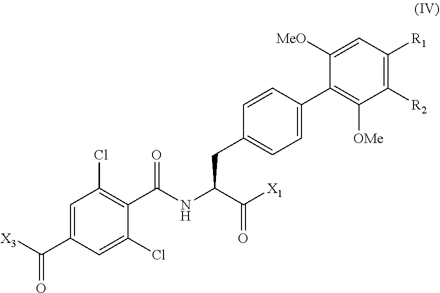

wherein:
R₁ is hydrogen, aminocarbonyl, carboxy, cyano, halo, hydroxy, —SF₅, alkyl_{(C≤8)}, substituted alkyl_{(C≤8)}, alkoxy_{(C≤8)}, substituted alkoxy_{(C≤8)}, or —Y₁—Rₐ; wherein:
   Y₁ is alkanediyl_{(C≤8)} or substituted alkanediyl_{(C≤8)}; and
   Rₐ is alkoxy_{(C≤12)}, acyloxy_{(C≤12)}, or a substituted version of either of these groups; or
—X(CH₂O)ₘ—(CH₂CH₂O)ₙ—R_{b}; wherein:
   X is a covalent bond or —O—;
   m is 0 or 1;
   n is 1, 2, 3, 4, 5, 6, 7, or 8; and
   R_{b} is hydrogen, alkyl_{(C≤8)}, or substituted alkyl_{(C≤8)}; or
—CH₂—O—(CH₂)ₙ—R_{c}; wherein:
   N is 0, 1, or 2; and
   R_{c} is a cyclic aliphatic ring of 4-6 in size having one oxygen as part of the ring;
R₂ is aminocarbonyl, carboxy, cyano, halo, hydroxy, —SF₅, alkyl_{(C≤8)}, substituted alkyl_{(C≤8)}, alkoxy_{(C≤8)}, substituted alkoxy_{(C≤8)}, or —Y₂—R_{c}; wherein:
   Y₂ is alkanediyl_{(C≤8)} or substituted alkanediyl_{(C≤8)}; and
   R_{c} is alkoxy_{(C≤12)}, acyloxy_{(C≤12)}, or a substituted version of either of these groups;
X₁ and X₃ are each independently hydroxy, alkoxy_{(C≤8)}, substituted alkoxy_{(C≤8)}, cycloalkoxy_{(C≤8)}, substituted cycloalkoxy_{(C≤8)}, alkenyloxy_{(C≤8)}, substituted alkenyloxy_{(C≤8)}, aryloxy_{(C≤8)}, substituted aryloxy_{(C≤8)}, aralkoxy_{(C≤8)}, substituted aralkoxy_{(C≤8)}, or a group convertible in vivo to hydroxy;
   or a pharmaceutically acceptable salt thereof.

In some embodiments, the compounds are further defined as:

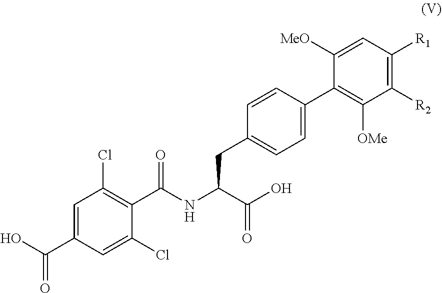

wherein:
R₁ is hydrogen, aminocarbonyl, carboxy, cyano, halo, hydroxy, —SF₅, alkyl_{(C≤8)}, substituted alkyl_{(C≤8)}, alkoxy_{(C≤8)}, substituted alkoxy_{(C≤8)}, or —Y₁—Rₐ; wherein:
   Y₁ is alkanediyl_{(C≤8)} or substituted alkanediyl_{(C≤8)}; and
   Rₐ is alkoxy_{(C≤12)}, acyloxy_{(C≤12)}, or a substituted version of either of these groups; or
—X(CH₂O)ₘ—(CH₂CH₂O)ₙ—R_{b}; wherein:
   X is a covalent bond or —O—;
   m is 0 or 1;
   n is 1, 2, 3, 4, 5, 6, 7, or 8; and
   R_{b} is hydrogen, alkyl_{(C≤8)}, or substituted alkyl_{(C≤8)}; or
—CH₂—O—(CH₂)ₙ—R_{c}; wherein:
   N is 0, 1, or 2; and
   R_{c} is a cyclic aliphatic ring of 4-6 in size having one oxygen as part of the ring;
R₂ is hydrogen, aminocarbonyl, carboxy, cyano, halo, hydroxy, —SF₅, alkyl_{(C≤8)}, substituted alkyl_{(C≤8)}, alkoxy_{(C≤8)}, substituted alkoxy_{(C≤8)}, or —Y₂—R_{c}; wherein:
   Y₂ is alkanediyl_{(C≤8)} or substituted alkanediyl_{(C≤8)}; and
   R_{c} is alkoxy_{(C≤12)}, acyloxy_{(C≤12)}, or a substituted version of either of these groups;
provided that R₁ and R₂ are not both hydrogen;
   or a pharmaceutically acceptable salt thereof.

In some embodiments, the compounds are further defined as:

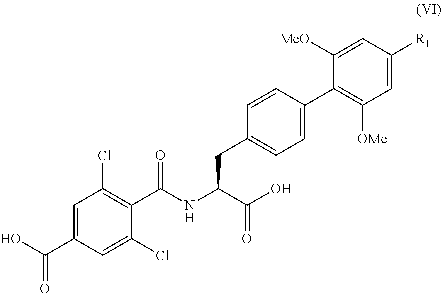

wherein:
R₁ is alkyl_{(C≤8)}, substituted alkyl_{(C≤8)}, alkoxy_{(C≤8)}, substituted alkoxy_{(C≤8)}, or —Y₁—Rₐ; wherein:
   Y₁ is alkanediyl_{(C≤8)} or substituted alkanediyl_{(C≤8)}; and
   Rₐ is alkoxy_{(C≤12)}, acyloxy_{(C≤12)}, or a substituted version of either of these groups; or
—X(CH₂O)ₘ—(CH₂CH₂O)ₙ—R_{b}; wherein:
   X is a covalent bond or —O—;
   m is 0 or 1;
   n is 1, 2, 3, 4, 5, 6, 7, or 8; and
   R_{b} is hydrogen, alkyl_{(C≤8)}, or substituted alkyl_{(C≤8)}; or
—CH₂—O—(CH₂)ₙ—R_{c}; wherein:
   N is 0, 1, or 2; and
   R_{c} is a cyclic aliphatic ring of 4-6 in size having one oxygen as part of the ring;
      or a pharmaceutically acceptable salt thereof.

In other embodiments, the compounds are further defined as:

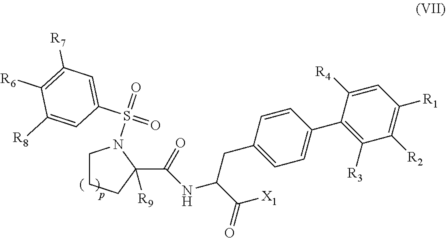

wherein:
R₁ is hydrogen, aminocarbonyl, carboxy, cyano, halo, hydroxy, —SF₅, alkyl_{(C≤8)}, substituted alkyl_{(C≤8)}, alkoxy_{(C≤8)}, substituted alkoxy_{(C≤8)}, or —Y₁—Rₐ, wherein:
   Y₁ is alkanediyl_{(C≤8)} or substituted alkanediyl_{(C≤8)}; and
   Rₐ is alkoxy_{(C≤12)}, acyloxy_{(C≤12)}, or a substituted version of either group; or
—X(CH₂O)ₘ—(CH₂CH₂O)ₙ—R_{b}, wherein:
   X is a covalent bond or —O—;
   m is 0 or 1;
   n is 1, 2, 3, 4, 5, 6, 7, or 8; and
   R_{b} is hydrogen, alkyl_{(C≤8)}, or substituted alkyl_{(C≤8)}; or
—CH₂—O—(CH₂)ₙ—R_{c}; wherein:
   N is 0, 1, or 2; and
   R_{c} is a cyclic aliphatic ring of 4-6 in size having one oxygen as part of the ring;
R₂ is hydrogen, aminocarbonyl, carboxy, cyano, halo, hydroxy, —SF₅, alkyl_{(C≤8)}, substituted alkyl_{(C≤8)}, alkoxy_{(C≤8)}, substituted alkoxy_{(C≤8)}, or —Y₂—R_{c}, wherein:
   Y₂ is alkanediyl_{(C≤8)} or substituted alkanediyl_{(C≤8)}; and
   R_{c} is alkoxy_{(C≤12)}, acyloxy_{(C≤12)}, or a substituted version of either group; or
R₃ and R₄ are each independently alkoxy_{(C≤8)} or substituted alkoxy_{(C≤8)};
R₇ and R₈ are each independently hydrogen, halo, haloalkyl_{(C≤8)}, or substituted haloalkyl_{(C≤8)};
R₆ is hydrogen or —C(O)X₂;
R₉ is hydrogen, alkyl_{(C≤6)}, or substituted alkyl_{(C≤6)};
p is 0, 1, 2, or 3; and
X₁ and X₂ are each independently hydroxy, alkoxy_{(C≤8)}, substituted alkoxy_{(C≤8)}, cycloalkoxy_{(C≤8)}, substituted cycloalkoxy_{(C≤8)}, alkenyloxy_{(C≤8)}, substituted alkenyloxy_{(C≤8)}, aryloxy_{(C≤8)}, substituted aryloxy_{(C≤8)}, aralkyloxy_{(C≤8)}, substituted aralkyloxy_{(C≤8)}, or a substituent convertible in vivo to hydroxy;
provided that R₁ and R₇ are not both hydrogen;
   or a pharmaceutically acceptable salt thereof.

In some embodiments, the compounds are further defined as:

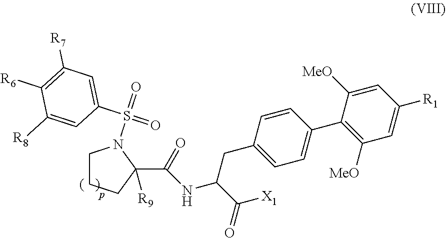

wherein:
R₁ is hydrogen, aminocarbonyl, carboxy, cyano, halo, hydroxy, —SF₅, alkyl_{(C≤8)}, substituted alkyl_{(C≤8)}, alkoxy_{(C≤8)}, substituted alkoxy_{(C≤8)}, or —Y₁—Rₐ, wherein:
   Y₁ is alkanediyl_{(C≤8)} or substituted alkanediyl_{(C≤8)}; and
   Rₐ is alkoxy_{(C≤12)}, acyloxy_{(C≤12)}, or a substituted version of either group; or
—X(CH₂O)ₘ—(CH₂CH₂O)ₙ—R_{b}, wherein:
   X is a covalent bond or —O—;
   m is 0 or 1;
   n is 1, 2, 3, 4, 5, 6, 7, or 8; and
   R_{b} is hydrogen, alkyl_{(C≤8)}, or substituted alkyl_{(C≤8)}; or
—CH₂—O—(CH₂)ₙ—R_{c}; wherein:
   N is 0, 1, or 2; and
   R_{c} is a cyclic aliphatic ring of 4-6 in size having one oxygen as part of the ring;
R₇ and R₈ are each independently hydrogen, halo, haloalkyl_{(C≤8)}, or substituted haloalkyl_{(C≤8)};
R₆ is hydrogen or —C(O)X₂;
R₉ is hydrogen, alkyl_{(C≤6)}, or substituted alkyl_{(C≤6)};
p is 0, 1, 2, or 3; and
X₁ and X₂ are each independently hydroxy, alkoxy_{(C≤8)}, substituted alkoxy_{(C≤8)}, cycloalkoxy_{(C≤8)}, substituted cycloalkoxy_{(C≤8)}, alkenyloxy_{(C≤8)}, substituted alkenyloxy_{(C≤8)}, aryloxy_{(C≤8)}, substituted aryloxy_{(C≤8)}, aralkyloxy_{(C≤8)}, substituted aralkyloxy_{(C≤8)}, or a substituent convertible in vivo to hydroxy;
provided that R₁ and R₇ are not both hydrogen;
   or a pharmaceutically acceptable salt thereof.

In some embodiments, the compounds are further defined as:

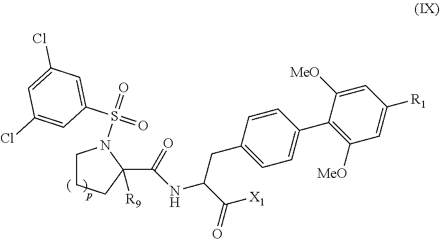

wherein:
R₁ is aminocarbonyl, carboxy, cyano, halo, hydroxy, —SF₅, alkyl_{(C≤8)}, substituted alkyl_{(C≤8)}, alkoxy_{(C≤8)}, substituted alkoxy_{(C≤8)}, or —Y₁—Rₐ, wherein:
   Y₁ is alkanediyl_{(C≤8)} or substituted alkanediyl_{(C≤8)}; and
   Rₐ is alkoxy_{(C≤12)}, acyloxy_{(C≤12)}, or a substituted version of either group; or
—X(CH₂O)ₘ—(CH₂CH₂O)ₙ—R_{b}, wherein:
   X is a covalent bond or —O—;
   m is 0 or 1;
   n is 1, 2, 3, 4, 5, 6, 7, or 8; and
   R_{b} is hydrogen, alkyl_{(C≤8)}, or substituted alkyl_{(C≤8)}; or
—CH₂—O—(CH₂)ₙ—R_{c}; wherein:
   N is 0, 1, or 2; and
   R_{c} is a cyclic aliphatic ring of 4-6 in size having one oxygen as part of the ring;
R₉ is hydrogen, alkyl_{(C≤6)}, or substituted alkyl_{(C≤6)};
p is 0, 1, 2, or 3; and
X₁ is hydroxy, alkoxy_{(C≤8)}, substituted alkoxy_{(C≤8)}, cycloalkoxy_{(C≤8)}, substituted cycloalkoxy_{(C≤8)}, alkenyloxy_{(C≤8)}, substituted alkenyloxy_{(C≤8)}, aryloxy_{(C≤8)}, substituted aryloxy_{(C≤8)}, aralkyloxy_{(C≤8)}, substituted aralkyloxy_{(C≤8)}, or a substituent convertible in vivo to hydroxy;
   or a pharmaceutically acceptable salt thereof.

In some embodiments, the compounds are further defined as:

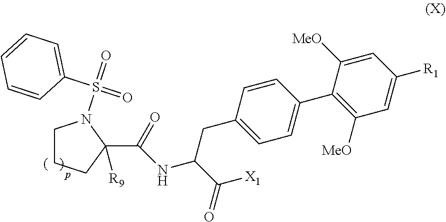

wherein:
R₁ is aminocarbonyl, carboxy, cyano, halo, hydroxy, —SF₅, alkyl_{(C≤8)}, substituted alkyl_{(C≤8)}, alkoxy_{(C≤8)}, substituted alkoxy_{(C≤8)}, or —Y₁—Rₐ, wherein:
   Y₁ is alkanediyl_{(C≤8)} or substituted alkanediyl_{(C≤8)}; and
   Rₐ is alkoxy_{(C≤12)}, acyloxy_{(C≤12)}, or a substituted version of either group; or
—X(CH₂O)ₘ—(CH₂CH₂O)ₙ—R_{b}, wherein:
   X is a covalent bond or —O—;
   m is 0 or 1;
   n is 1, 2, 3, 4, 5, 6, 7, or 8; and
   R_{b} is hydrogen, alkyl_{(C≤8)}, or substituted alkyl_{(C≤8)}; or
—CH₂—O—(CH₂)ₙ—R_{c}; wherein:
   N is 0, 1, or 2; and
   R_{c} is a cyclic aliphatic ring of 4-6 in size having one oxygen as part of the ring;
R₉ is hydrogen, alkyl_{(C≤6)}, or substituted alkyl_{(C≤6)};
p is 0, 1, 2, or 3; and
X₁ is hydroxy, alkoxy_{(C≤8)}, substituted alkoxy_{(C≤8)}, cycloalkoxy_{(C≤8)}, substituted cycloalkoxy_{(C≤8)}, alkenyloxy_{(C≤8)}, substituted alkenyloxy_{(C≤8)}, aryloxy_{(C≤8)}, substituted aryloxy_{(C≤8)}, aralkyloxy_{(C≤8)}, substituted aralkyloxy_{(C≤8)}, or a substituent convertible in vivo to hydroxy;
   or a pharmaceutically acceptable salt thereof. In some embodiments, the compounds are further defined as:

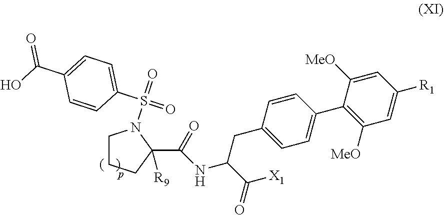

wherein:
R₁ is aminocarbonyl, carboxy, cyano, halo, hydroxy, —SF₅, alkyl_{(C≤8)}, substituted alkyl_{(C≤8)}, alkoxy_{(C≤8)}, substituted alkoxy_{(C≤8)}, or —Y₁—Rₐ, wherein:
   Y₁ is alkanediyl_{(C≤8)} or substituted alkanediyl_{(C≤8)}; and
   Rₐ is alkoxy_{(C≤12)}, acyloxy_{(C≤12)}, or a substituted version of either group; or
—X(CH₂O)ₘ—(CH₂CH₂O)ₙ—R_{b}, wherein:
   X is a covalent bond or —O—;
   m is 0 or 1;
   n is 1, 2, 3, 4, 5, 6, 7, or 8; and
   R_{b} is hydrogen, alkyl_{(C≤8)}, or substituted alkyl_{(C≤8)}; or
—CH₂—O—(CH₂)ₙ—R_{c}; wherein:
   N is 0, 1, or 2; and
   R_{c} is a cyclic aliphatic ring of 4-6 in size having one oxygen as part of the ring;
R₉ is hydrogen, alkyl_{(C≤6)}, or substituted alkyl_{(C≤6)};
p is 0, 1, 2, or 3; and
X₁ is hydroxy, alkoxy_{(C≤8)}, substituted alkoxy_{(C≤8)}, cycloalkoxy_{(C≤8)}, substituted cycloalkoxy_{(C≤8)}, alkenyloxy_{(C≤8)}, substituted alkenyloxy_{(C≤8)}, aryloxy_{(C≤8)}, substituted aryloxy_{(C≤8)}, aralkyloxy_{(C≤8)}, substituted aralkyloxy_{(C≤8)}, or a substituent convertible in vivo to hydroxy;
   or a pharmaceutically acceptable salt thereof.

In other embodiments, the compounds are further defined as:

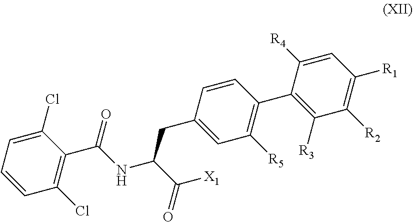

wherein:
R₁ is aminocarbonyl, carboxy, cyano, halo, hydroxy, —SF₅, alkyl_{(C≤8)}, substituted alkyl_{(C≤8)}, alkoxy_{(C≤8)}, substituted alkoxy_{(C≤8)}, or —Y₁—Rₐ, wherein:
   Y₁ is alkanediyl_{(C≤8)} or substituted alkanediyl_{(C≤8)}; and
   Rₐ is alkoxy_{(C≤12)}, acyloxy_{(C≤12)}, or a substituted version of either group; or
—X(CH₂O)ₘ—(CH₂CH₂O)ₙ—R_{b}, wherein:
   X is a covalent bond or —O—;
   m is 0 or 1;
   n is 1, 2, 3, 4, 5, 6, 7, or 8; and
   R_{b} is hydrogen, alkyl_{(C≤8)}, or substituted alkyl_{(C≤8)}; or
—CH₂—O—(CH₂)ₙ—R_{c}; wherein:
   N is 0, 1, or 2; and
   R_{c} is a cyclic aliphatic ring of 4-6 in size having one oxygen as part of the ring;
R₂ is hydrogen, aminocarbonyl, carboxy, cyano, halo, hydroxy, —SF₅, alkyl_{(C≤8)}, substituted alkyl_{(C≤8)}, alkoxy_{(C≤8)}, substituted alkoxy_{(C≤8)}, or —Y₂—R_{c}, wherein:
   Y₂ is alkanediyl_{(C≤8)} or substituted alkanediyl_{(C≤8)}; and
   R_{c} is alkoxy_{(C≤12)}, alkoxy_{(C≤12)}, or a substituted version of either group;
R₃ and R₄ are each independently alkoxy_{(C≤8)} or substituted alkoxy_{(C≤8)};
R₅ is hydrogen, —CH(OR_{d})Rₑ, or —C(O)R_{f}, wherein:
   R_{d} is hydrogen, alkyl_{(C≤8)}, substituted alkyl_{(C≤8)}, acyl_{(C≤8)}, or substituted acyl_{(C≤8)}; and
   Rₑ and R_{f} are each independently alkyl_{(C≤8)} or substituted alkyl_{(C≤8)};
X₁ is hydroxy, alkoxy_{(C≤8)}, substituted alkoxy_{(C≤8)}, cycloalkoxy_{(C≤8)}, substituted cycloalkoxy_{(C≤8)}, alkenyloxy_{(C≤8)}, substituted alkenyloxy_{(C≤8)}, aryloxy_{(C≤8)}, substituted aryloxy_{(C≤8)}, aralkyloxy_{(C≤8)}, substituted aralkyloxy_{(C≤8)}, or a substituent convertible in vivo to hydroxy;
   or a pharmaceutically acceptable salt thereof.

In some embodiments, the compounds are further defined as:

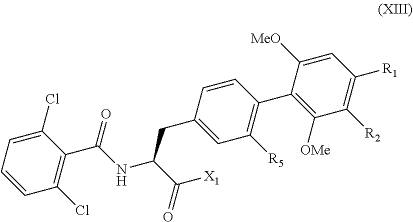

wherein:
R₁ is aminocarbonyl, carboxy, cyano, halo, hydroxy, —SF₅, alkyl_{(C≤8)}, substituted alkyl_{(C≤8)}, alkoxy_{(C≤8)}, substituted alkoxy_{(C≤8)}, or —Y₁—Rₐ, wherein:
   Y₁ is alkanediyl_{(C≤8)} or substituted alkanediyl_{(C≤8)}; and
   Rₐ is alkoxy_{(C≤12)}, acyloxy_{(C≤12)}, or a substituted version of either group; or
—X(CH₂O)ₘ—(CH₂CH₂O)ₙ—R_{b}, wherein:
   X is a covalent bond or —O—;
   m is 0 or 1;
   n is 1, 2, 3, 4, 5, 6, 7, or 8; and
   R_{b} is hydrogen, alkyl_{(C≤8)}, or substituted alkyl_{(C≤8)}; or
—CH₂—O—(CH₂)ₙ—R_{c}; wherein:
   N is 0, 1, or 2; and
   R_{c} is a cyclic aliphatic ring of 4-6 in size having one oxygen as part of the ring;
R₂ is hydrogen, aminocarbonyl, carboxy, cyano, halo, hydroxy, —SF₅, alkyl_{(C≤8)}, substituted alkyl_{(C≤8)}, alkoxy_{(C≤8)}, substituted alkoxy_{(C≤8)}, or —Y₂—R_{c}, wherein:
   Y₂ is alkanediyl_{(C≤8)} or substituted alkanediyl_{(C≤8)}; and
   R_{c} is alkoxy_{(C≤12)}, alkoxy_{(C≤12)}, or a substituted version of either group;
R₅ is hydrogen, —CH(OR_{d})Rₑ, or —C(O)R_{f}, wherein:
   R_{d} is hydrogen, alkyl_{(C≤8)}, substituted alkyl_{(C≤8)}, acyl_{(C≤8)}, or substituted acyl_{(C≤8)}; and
   Rₑ and R_{f} are each independently alkyl_{(C≤8)} or substituted alkyl_{(C≤8)};
X₁ is hydroxy, alkoxy_{(C≤8)}, substituted alkoxy_{(C≤8)}, cycloalkoxy_{(C≤8)}, substituted cycloalkoxy_{(C≤8)}, alkenyloxy_{(C≤8)}, substituted alkenyloxy_{(C≤8)}, aryloxy_{(C≤8)}, substituted aryloxy_{(C≤8)}, aralkyloxy_{(C≤8)}, substituted aralkyloxy_{(C≤8)}, or a substituent convertible in vivo to hydroxy;
   or a pharmaceutically acceptable salt thereof.

In some embodiments, the compounds are further defined as:

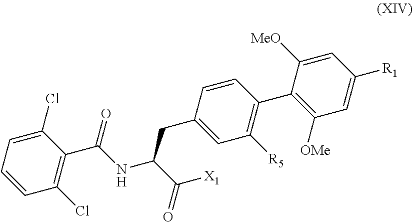

wherein:
R₁ is aminocarbonyl, carboxy, cyano, halo, hydroxy, —SF₅, alkyl_{(C≤8)}, substituted alkyl_{(C≤8)}, alkoxy_{(C≤8)}, substituted alkoxy_{(C≤8)}, or —Y₁—Rₐ, wherein:
   Y₁ is alkanediyl_{(C≤8)} or substituted alkanediyl_{(C≤8)}; and
   Rₐ is alkoxy_{(C≤12)}, acyloxy_{(C≤12)}, or a substituted version of either group; or
—X(CH₂O)ₘ—(CH₂CH₂O)ₙ—R_{b}, wherein:
   X is a covalent bond or —O—;
   m is 0 or 1;
   n is 1, 2, 3, 4, 5, 6, 7, or 8; and
   R_{b} is hydrogen, alkyl_{(C≤8)}, or substituted alkyl_{(C≤8)}; or
—CH₂—O—(CH₂)ₙ—R_{c}; wherein:
   N is 0, 1, or 2; and
   R_{c} is a cyclic aliphatic ring of 4-6 in size having one oxygen as part of the ring;
R₅ is hydrogen, —CH(OR_{d})Rₑ, or —C(O)R_{f}, wherein:
   R_{d} is hydrogen, alkyl_{(C≤8)}, substituted alkyl_{(C≤8)}, acyl_{(C≤8)}, or substituted acyl_{(C≤8)}; and
   Rₑ and R_{f} are each independently alkyl_{(C≤8)} or substituted alkyl_{(C≤8)};
X₁ is hydroxy, alkoxy_{(C≤8)}, substituted alkoxy_{(C≤8)}, cycloalkoxy_{(C≤8)}, substituted cycloalkoxy_{(C≤8)}, alkenyloxy_{(C≤8)}, substituted alkenyloxy_{(C≤8)}, aryloxy_{(C≤8)}, substituted aryloxy_{(C≤8)}, aralkyloxy_{(C≤8)}, substituted aralkyloxy_{(C≤8)}, or a substituent convertible in vivo to hydroxy;
   or a pharmaceutically acceptable salt thereof.

In some embodiments, Z is:

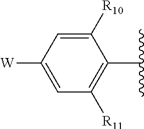

wherein:
W is hydrogen, cyano, halo, hydroxy, or —C(O)X₃, wherein:
   X₃ is amino, hydroxy, alkoxy_{(C≤8)}, substituted alkoxy_{(C≤8)}, cycloalkoxy_{(C≤8)}, substituted cycloalkoxy_{(C≤8)}, alkenyloxy_{(C≤8)}, substituted alkenyloxy_{(C≤8)}, aryloxy_{(C≤8)}, substituted aryloxy_{(C≤8)}, aralkyloxy_{(C≤8)}, substituted aralkyloxy_{(C≤8)}, alkylamino_{(C≤8)}, substituted alkylamino_{(C≤8)}, dialkylamino_{(C≤8)}, substituted dialkylamino_{(C≤8)}, cycloalkylamino_{(C≤8)}, substituted cycloalkylamino_{(C≤8)}, alkenylamino_{(C≤8)}, substituted alkenyl-amino_{(C≤8)}, arylamino_{(C≤8)}, substituted arylamino_{(C≤8)}, aralkylamino_{(C≤8)}, substituted aralkylamino_{(C≤8)}, or a substituent convertible in vivo to hydroxy; and
R₁₀ and R₁₁ are each independently hydrogen or halo.

In some embodiments, Z is:

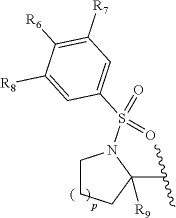

wherein:
p is 0, 1, 2, or 3;
R₆ is hydrogen or —C(O)X₂; wherein:
   X₂ is amino, hydroxy, alkoxy_{(C≤8)}, substituted alkoxy_{(C≤8)}, cycloalkoxy_{(C≤8)}, substituted cycloalkoxy_{(C≤8)}, alkenyloxy_{(C≤8)}, substituted alkenyloxy_{(C≤8)}, aryloxy_{(C≤8)}, substituted aryloxy_{(C≤8)}, aralkyloxy_{(C≤8)}, substituted aralkyloxy_{(C≤8)}, alkylamino_{(C≤8)}, substituted alkylamino_{(C≤8)}, dialkylamino_{(C≤8)}, substituted dialkylamino_{(C≤8)}, cycloalkylamino_{(C≤8)}, substituted cycloalkyl-amino_{(C≤8)}, alkenylamino_{(C≤8)}, substituted alkenylamino_{(C≤8)}, arylamino_{(C≤8)}, substituted arylamino_{(C≤8)}, aralkylamino_{(C≤8)}, substituted aralkylamino_{(C≤8)}, or a substituent convertible in vivo to hydroxy;
R₇ and R₈ are each independently hydrogen, halo, haloalkyl_{(C≤8)}, or substituted haloalkyl_{(C≤8)}; and
R₉ is hydrogen, alkyl_{(C≤8)}, or substituted alkyl_{(C≤8)}.

In some embodiments, R₁ is alkyl_{(C≤8)} or substituted alkyl_{(C≤8)} such as hydroxyalkyl_{(C≤8)} or haloalkyl_{(C≤8)}. In some embodiments, R₁ is an unbranched group. In some embodiments, R₁ is —CH₃, —CH₂OH, —CH(CH₃)OH, —C(CH₃)₂OH, —CH₂F, —CHF₂, —CF₃, —CH₂OCH₃, or —CH₂OCH₂CH₃. In some embodiments, R₁ is alkoxy_{(C≤8)} or substituted alkoxy_{(C≤8)}, such as methoxy or ethoxy. In other embodiments, R₁ is aminocarbonyl or carboxy. In other embodiments, R₁ is aminocarbonyl or carboxy. In other embodiments, R1 is —CH₂—O—(CH2)ₙ—R_{c}; wherein N is 0, 1, or 2; and R_{c} is a cyclic aliphatic ring of 4-6 in size having one oxygen as part of the ring

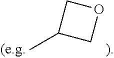

In other embodiments, R₁ is hydroxy, alkyl_{(C≤8)}, substituted alkyl_{(C≤8)}, alkoxy_{(C≤8)}, substituted alkoxy_{(C≤8)}, or —Y₁—Rₐ, wherein:
Y₁ is alkanediyl_{(C≤8)} or substituted alkanediyl_{(C≤8)}; and
Rₐ is alkoxy_{(C≤12)}, acyloxy_{(C≤12)}, or a substituted version of either group; or —X(CH₂O)ₘ—(CH₂CH₂O)ₙ—R_{b}, wherein:
X is a covalent bond or —O—;
m is 0 or 1;
n is 1, 2, 3, 4, 5, 6, 7, or 8; and
R_{b} is hydrogen, alkyl_{(C≤8)}, or substituted alkyl_{(C≤8)}.

In other embodiments, R₁ is —Y₁—Rₐ, wherein: Y₁ is alkanediyl_{(C≤8)} or substituted alkanediyl_{(C≤8)} and Rₐ is alkoxy_{(C≤12)}, acyloxy_{(C≤12)}, or a substituted version of either group. In some embodiments, Y₁ is alkanediyl_{(C≤8)}, such as —CH₂—. In some embodiments, Rₐ is alkoxy_{(C≤12)}, such as ethoxy or hexyloxy. In some embodiments, Rₐ is acyloxy_{(C≤12)}, such as hexanoate. In other embodiments, R₁ is —X(CH₂O)ₘ—(CH₂CH₂O)ₙ—R_{b}, wherein:
X is a covalent bond or —O—;
m is 0 or 1;
n is 1, 2, 3, 4, 5, 6, 7, or 8; and
R_{b} is hydrogen, alkyl_{(C≤8)}, or substituted alkyl_{(C≤8)}.

In some embodiments, X is a covalent bond. In some embodiments, m is 1. In some embodiments, n is 1, 2, or 3. In some embodiments, n is 1. In some embodiments, R_{b} is alkyl_{(C≤8)}, such as methyl. In some embodiments, R₂ is hydrogen. In some embodiments, R₃ is alkoxy_{(C≤6)}, such as methoxy. In some embodiments, R₄ is alkoxy_{(C≤6)}, such as methoxy. In some embodiments, X₁ is hydroxy. In other embodiments, X₁ is a substituent convertible in vivo to hydroxy. In some embodiments, X₂ is hydroxy. In other embodiments, X₂ is a substituent convertible in vivo to hydroxy. In some embodiments, R₇ is hydrogen. In other embodiments, R₇ is halo, such as chloro. In some embodiments, R₈ is hydrogen. In other embodiments, R₈ is halo, such as chloro. In some embodiments, R₆ is hydrogen. In some embodiments, R₉ is hydrogen. In other embodiments, R₉ is alkyl_{(C≤8)}. In some embodiments, R₉ is alkyl_{(C≤4)}, such as methyl.

In some embodiments, p is 0, 1, or 2. In some embodiments, p is 1 or 2. In some embodiments, R₁₀ is halo, such as chloro. In some embodiments, R₁₁ is halo, such as chloro. In some embodiments, W is hydrogen. In other embodiments, W is —C(O)X₃. In other embodiments, X₃ is hydroxy. In other embodiments, X₃ is a substituent convertible in vivo to hydroxy.

In some embodiments, the compound is further defined as:

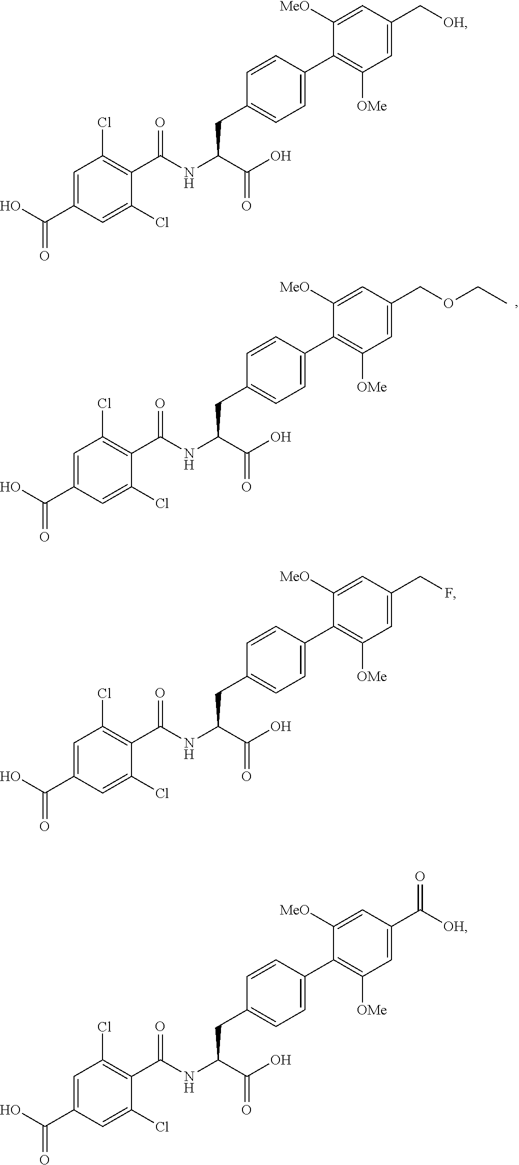 [SMILES: COc1cc(CO)cc(OC)c1-c1ccc(C[C@H](NC(=O)c2c(Cl)cc(C(=O)O)cc2Cl)C(=O)O)cc1 ; CCOCc1cc(OC)c(-c2ccc(C[C@H](NC(=O)c3c(Cl)cc(C(=O)O)cc3Cl)C(=O)O)cc2)c(OC)c1 ; COc1cc(CF)cc(OC)c1-c1ccc(C[C@H](NC(=O)c2c(Cl)cc(C(=O)O)cc2Cl)C(=O)O)cc1 ; COc1cc(C(=O)O)cc(OC)c1-c1ccc(C[C@H](NC(=O)c2c(Cl)cc(C(=O)O)cc2Cl)C(=O)O)cc1]

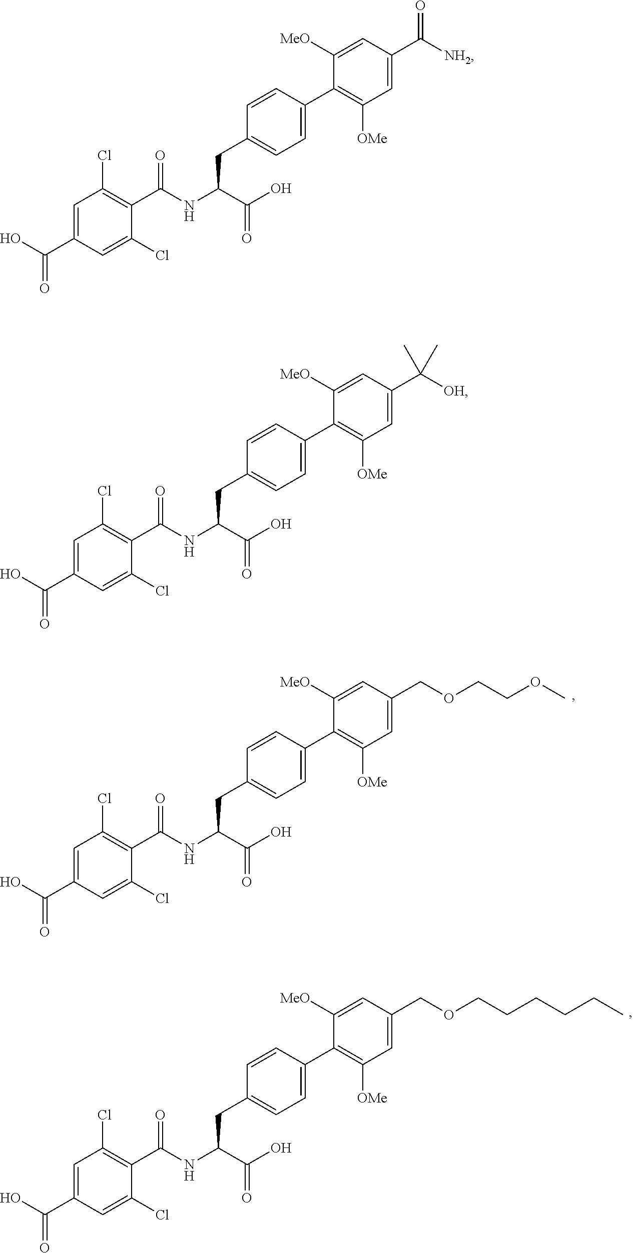 [SMILES: COc1cc(C(N)=O)cc(OC)c1-c1ccc(C[C@H](NC(=O)c2c(Cl)cc(C(=O)O)cc2Cl)C(=O)O)cc1 ; COc1cc(C(C)(C)O)cc(OC)c1-c1ccc(C[C@H](NC(=O)c2c(Cl)cc(C(=O)O)cc2Cl)C(=O)O)cc1 ; COCCOCc1cc(OC)c(-c2ccc(C[C@H](NC(=O)c3c(Cl)cc(C(=O)O)cc3Cl)C(=O)O)cc2)c(OC)c1 ; CCCCCCOCc1cc(OC)c(-c2ccc(C[C@H](NC(=O)c3c(Cl)cc(C(=O)O)cc3Cl)C(=O)O)cc2)c(OC)c1]

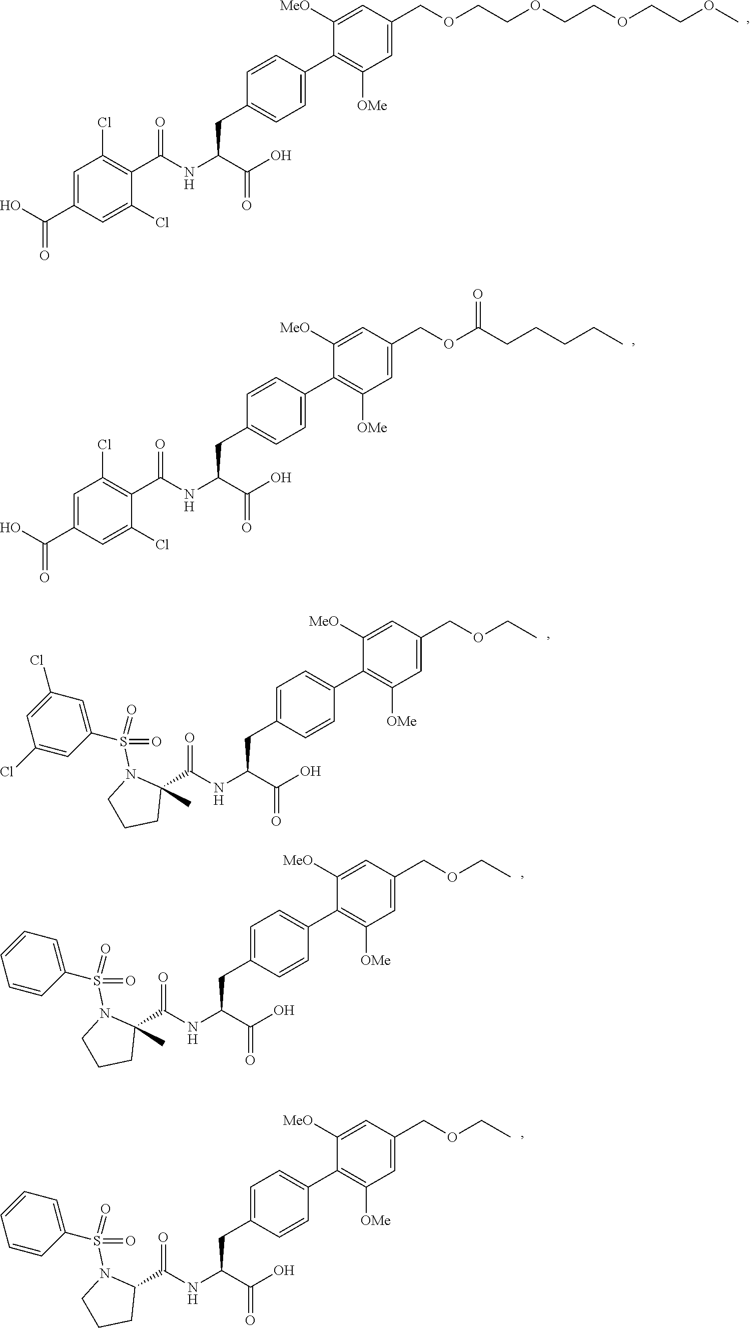 [SMILES: COCCOCCOCCOCc1cc(OC)c(-c2ccc(C[C@H](NC(=O)c3c(Cl)cc(C(=O)O)cc3Cl)C(=O)O)cc2)c(OC)c1 ; CCCCCC(=O)OCc1cc(OC)c(-c2ccc(C[C@H](NC(=O)c3c(Cl)cc(C(=O)O)cc3Cl)C(=O)O)cc2)c(OC)c1 ; CCOCc1cc(OC)c(-c2ccc(C[C@H](NC(=O)[C@]3(C)CCCN3S(=O)(=O)c3cc(Cl)cc(Cl)c3)C(=O)O)cc2)c(OC)c1 ; CCOCc1cc(OC)c(-c2ccc(C[C@H](NC(=O)[C@]3(C)CCCN3S(=O)(=O)c3ccccc3)C(=O)O)cc2)c(OC)c1 ; CCOCc1cc(OC)c(-c2ccc(C[C@H](NC(=O)[C@@H]3CCCN3S(=O)(=O)c3ccccc3)C(=O)O)cc2)c(OC)c1]

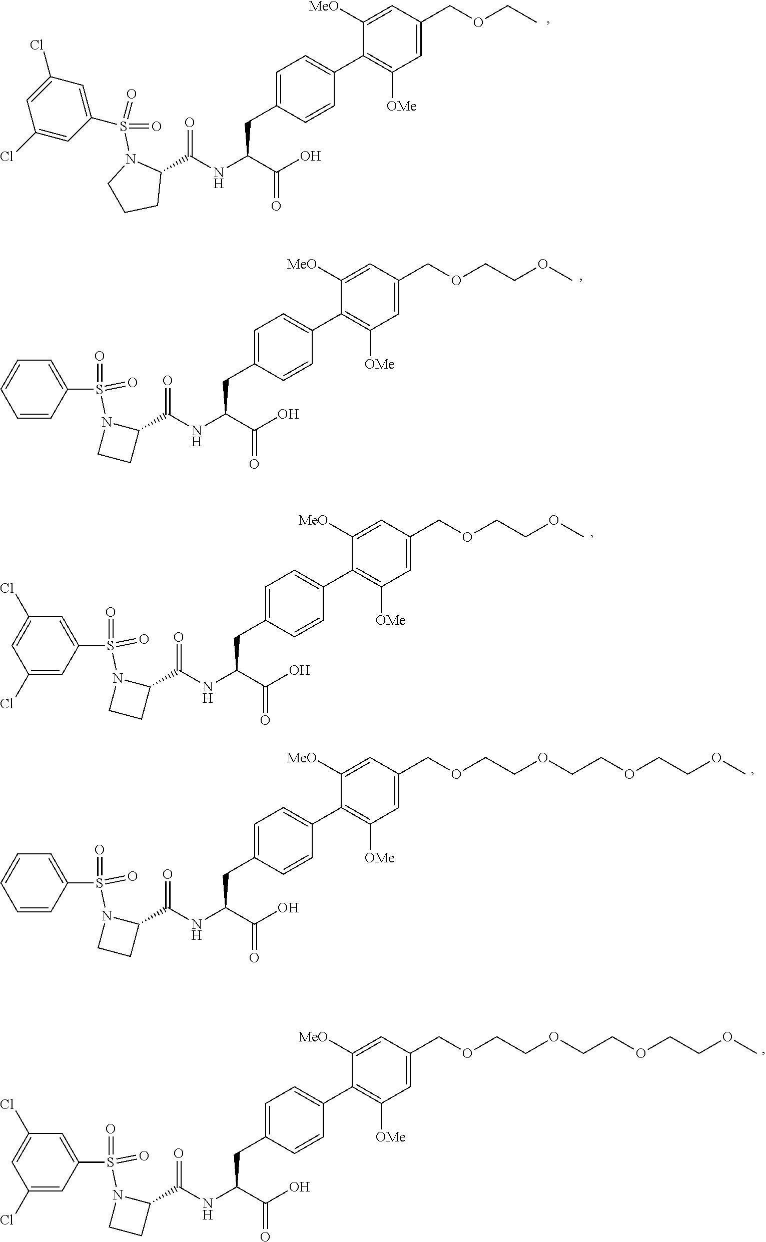 [SMILES: CCOCc1cc(OC)c(-c2ccc(C[C@H](NC(=O)[C@@H]3CCCN3S(=O)(=O)c3cc(Cl)cc(Cl)c3)C(=O)O)cc2)c(OC)c1 ; COCCOCc1cc(OC)c(-c2ccc(C[C@H](NC(=O)[C@@H]3CCN3S(=O)(=O)c3ccccc3)C(=O)O)cc2)c(OC)c1 ; COCCOCc1cc(OC)c(-c2ccc(C[C@H](NC(=O)[C@@H]3CCN3S(=O)(=O)c3cc(Cl)cc(Cl)c3)C(=O)O)cc2)c(OC)c1 ; COCCOCCOCCOCc1cc(OC)c(-c2ccc(C[C@H](NC(=O)[C@@H]3CCN3S(=O)(=O)c3ccccc3)C(=O)O)cc2)c(OC)c1 ; COCCOCCOCCOCc1cc(OC)c(-c2ccc(C[C@H](NC(=O)[C@@H]3CCN3S(=O)(=O)c3cc(Cl)cc(Cl)c3)C(=O)O)cc2)c(OC)c1]

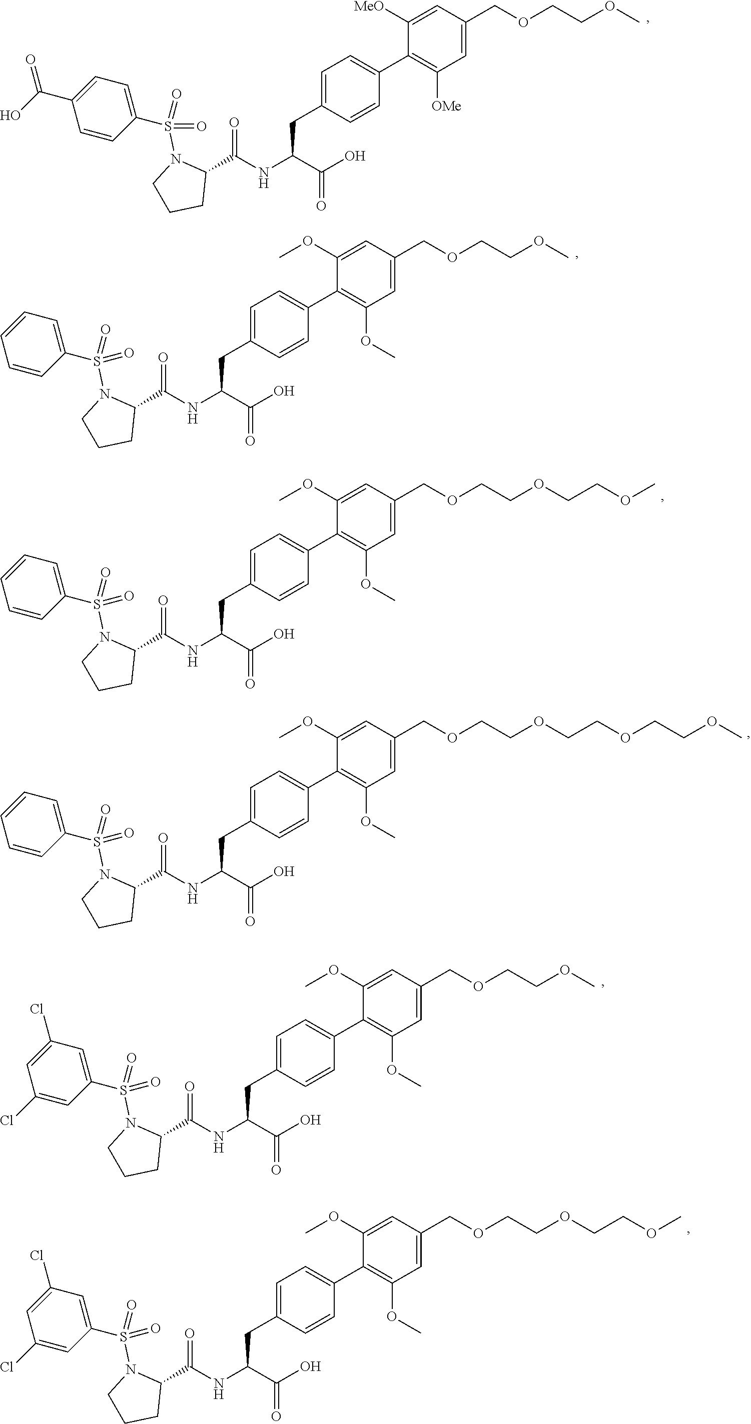

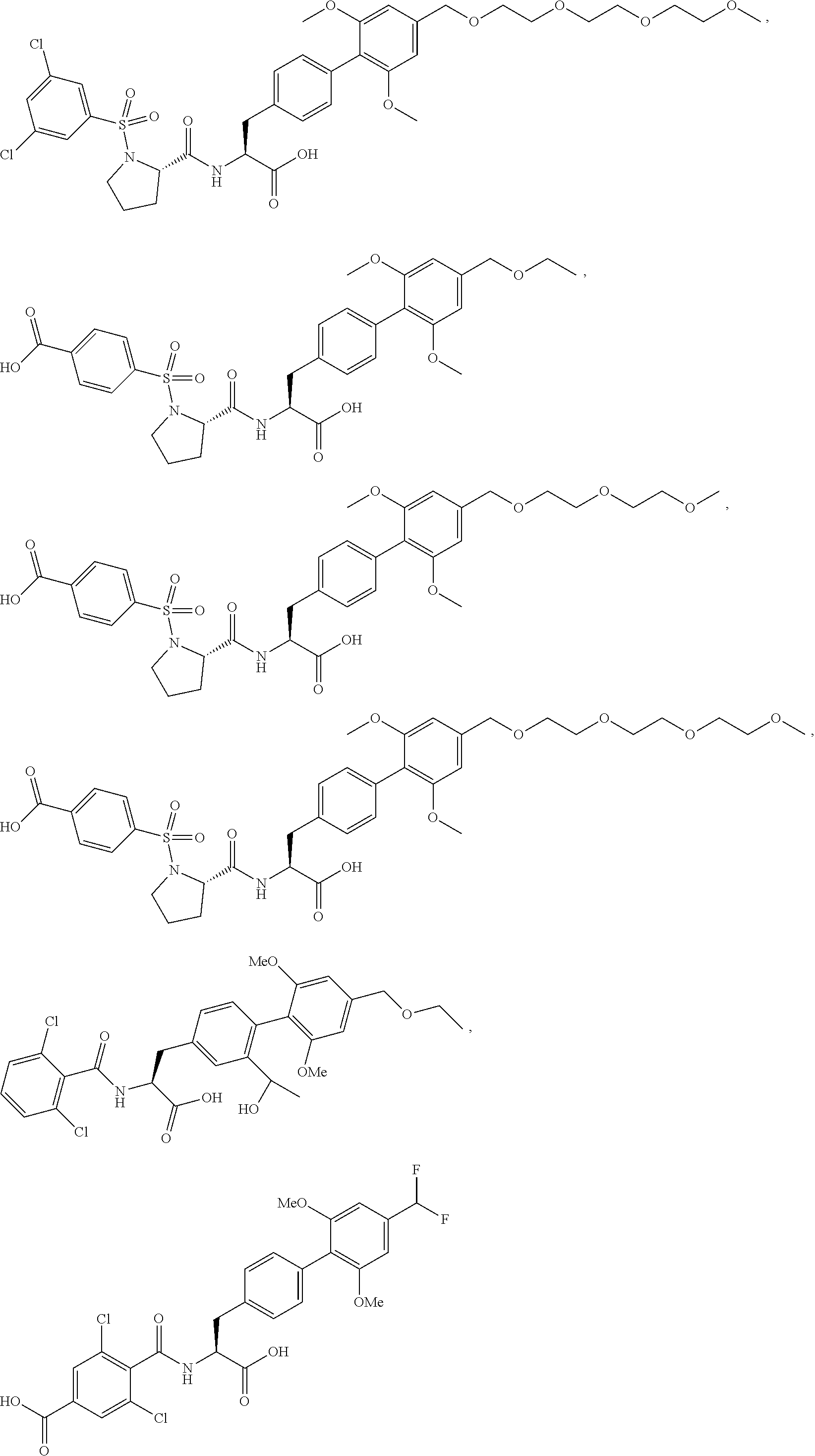

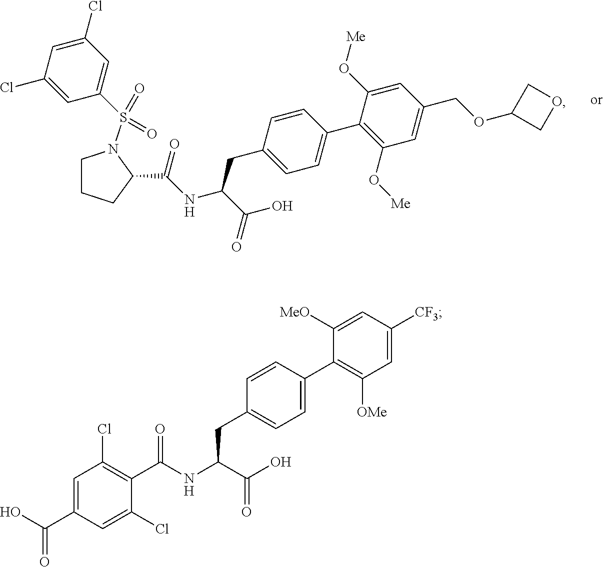 [SMILES: COc1cc(COC2COC2)cc(OC)c1-c1ccc(C[C@H](NC(=O)[C@@H]2CCCN2S(=O)(=O)c2cc(Cl)cc(Cl)c2)C(=O)O)cc1 ; "or" ; COc1cc(C(F)(F)F)cc(OC)c1-c1ccc(C[C@H](NC(=O)c2c(Cl)cc(C(=O)O)cc2Cl)C(=O)O)cc1]

or a pharmaceutically salt thereof.

In some embodiments, the agent which interacts with a chemokine receptor is an agent which interacts with a C—X—C chemokine receptor. In some embodiments, the agent is a CXCR2 agonist. In some embodiments, the agent is plerixafor, Groβ, or a derivative of Groβ. In some embodiments, the derivative of Groβ is a truncated Groβ. In some embodiments, the truncated Gro-β is SB-251353.

In some embodiments, the composition further comprises an inhibitor of integrin α9β1, G-CSF, a derivative of G-CSF, or a combination thereof. In some embodiments, the derivative of G-CSF is a pegylated G-CSF. In some embodiments, the inhibitor of integrin α9β1 is (N-benzenesulfonyl)-L-prolyl-L-O-(1-pyrrolidinylcarbonyl)tyrosine (BOP).

In some aspects, the present disclosure provides a pharmaceutical composition comprising a composition disclosed herein and a pharmaceutically acceptable excipient. In some embodiments, the pharmaceutical composition is formulated for oral administration, intraarterial administration, intraperitoneal administration, intravenous administration, or subcutaneous administration. In some embodiments, the pharmaceutical composition is formulated for administration via intravenous infusion. In other embodiments, the pharmaceutical composition is formulated for administration via subcutaneous injection. In some embodiments, the composition consists substantially of the agent which interacts with one or more chemokine receptors, the VLA-4 inhibitor, and the pharmaceutically acceptable excipient. In some embodiments, the composition consists essentially of the agent which interacts with one or more chemokine receptors, the VLA-4 inhibitor, and the pharmaceutically acceptable excipient.

Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description. Note that simply because a particular compound is ascribed to one particular generic formula does not mean that it cannot also belong to another generic formula.

### (b) Description of Illustrative Embodiments

Disclosed herein are methods comprising an agent which interacts with a chemokine receptor, such as a CXCR2 agonist, and a compound that act as an integrin antagonist or inhibitor, such as an α4β1 integrin (VLA-4) antagonist as well as pharmaceutical compositions thereof. These pharmaceutical compositions may result in the mobilization of progenitor and/or stem cells from bone marrow to peripheral circulation. Additionally, provided herein are methods for the treatment and/or prevention of disease using these two therapeutic agents in combination. These compositions described herein may be used to stimulate progenitor and/or stem cells (e.g. hematopoietic stem cells such as CD34+ hematopoietic stem cells) and result in such stimulation in a shorter amount of time relative to either agent alone or other known agents or combinations. These compositions may also have the added advantage that they result in mobilization in higher numbers, begin mobilization in a shorter period of time or over a more prolonged period of time, or mobilize increased numbers of early progenitor and/or stem cells, LSK-SLAM cells, CFU-C cells, or other progenitor and/or stem cells which are competent to achieve a successful engraftment into the patient. In some embodiments, these compositions may be used in improving the harvest of hematopoietic stem cells or progenitor cells. These methods, compositions, or uses are described in more detail below.

(i) Compounds and Synthetic Methods

In some embodiments, the compounds used in the compositions of the present disclosure include the compounds described in the Examples and claims listed below. All the synthesis methods described herein can be further modified and optimized using the principles and techniques of organic chemistry as applied by a person skilled in the art. Such principles and techniques are taught, for example, in March's Advanced Organic Chemistry: Reactions, Mechanisms, and Structure (2007), which is incorporated by reference herein.

Compounds employed in methods of the disclosure may contain one or more asymmetrically-substituted carbon or nitrogen atoms, and may be isolated in optically active or racemic form. Thus, all chiral, diastereomeric, racemic form, epimeric form, and all geometric isomeric forms of a structure are intended, unless the specific stereochemistry or isomeric form is specifically indicated. Compounds may occur as racemates and racemic mixtures, single enantiomers, diastereomeric mixtures and individual diastereomers. In some embodiments, a single diastereomer is obtained. The chiral centers of the compounds of the present disclosure can have the S or the R configuration, as defined by the IUPAC 1974 Recommendations. For example, mixtures of stereoisomers may be separated using the techniques taught in the Examples section below, as well as modifications thereof. Tautomeric forms are also included as well as pharmaceutically acceptable salts of such isomers and tautomers.

Atoms making up the compounds of the present disclosure are intended to include all isotopic forms of such atoms. Compounds of the present disclosure include those with one or more atoms that have been isotopically modified or enriched, in particular those with pharmaceutically acceptable isotopes or those useful for pharmaceutically research. Isotopes, as used herein, include those atoms having the same atomic number but different mass numbers. By way of general example and without limitation, isotopes of hydrogen include deuterium and tritium, and isotopes of carbon include ¹³C and ¹⁴C. Similarly, it is contemplated that one or more carbon atom(s) of a compound of the present disclosure may be replaced by a silicon atom(s). Furthermore, it is contemplated that one or more oxygen atom(s) of a compound of the present disclosure may be replaced by a sulfur or selenium atom(s).

Compounds of the present disclosure may also exist in prodrug form. Since prodrugs are known to enhance numerous desirable qualities of pharmaceuticals (e.g., solubility, bioavailability, manufacturing, etc.), the compounds employed in some methods of the disclosure may, if desired, be delivered in prodrug form. Thus, the disclosure contemplates prodrugs of compounds of the present disclosure as well as methods of delivering prodrugs. Prodrugs of the compounds employed in the disclosure may be prepared by modifying functional groups present in the compound in such a way that the modifications are cleaved, either in routine manipulation or in vivo, to the parent compound. Accordingly, prodrugs include, for example, compounds described herein in which a hydroxy, amino, or carboxy group is bonded to any group that, when the prodrug is administered to a subject, cleaves to form a hydroxy, amino, or carboxylic acid, respectively. Additional details regarding pro-drugs may be found in Smith and Williams, 1988, the entire contents of which are hereby incorporated by reference. Smith and Williams, 1988.

It should be recognized that the particular anion or cation forming a part of any salt of this disclosure is not critical, so long as the salt, as a whole, is pharmacologically acceptable. Compounds useful in the disclosure which are amines, may be administered or prepared in the forms of their acid addition salts or metal complexes thereof. Suitable acid addition salts include salts of inorganic acids that are biocompatible, including HCl, HBr, sulfuric, phosphoric and the like, as well as organic acids such as acetic, propionic, butyric and the like, as well as acids containing more than one carboxyl group, such as oxalic, glutaric, adipic and the like. Compounds useful in the disclosure that are carboxylic acids or otherwise acidic may be administered or prepared in forms of salts formed from inorganic or organic bases that are physiologically compatible. Thus, these compounds may be prepared in the forms of their sodium, potassium, calcium, or magnesium salts as appropriate or may be salts with organic bases such as caffeine or ethylamine. These compounds also may be in the form of metal complexes. Additional examples of pharmaceutically acceptable salts and their methods of preparation and use are presented in Handbook of Pharmaceutical Salts: Properties, and Use (2002), which is incorporated herein by reference.

It should be further recognized that the compounds of the present disclosure include those that have been further modified to comprise substituents that are convertible to hydrogen in vivo. This includes those groups that may be convertible to a hydrogen atom by enzymological or chemical means including, but not limited to, hydrolysis and hydrogenolysis. Examples include hydrolyzable groups, such as acyl groups, groups having an oxycarbonyl group, amino acid residues, peptide residues, o nitrophenylsulfenyl, trimethylsilyl, tetrahydropyranyl, diphenylphosphinyl, and the like. Examples of acyl groups include formyl, acetyl, trifluoroacetyl, and the like. Examples of groups having an oxycarbonyl group include ethoxycarbonyl, tert-butoxycarbonyl (—C(O)OC(CH₃)₃, Boc), benzyloxycarbonyl, p methoxy-benzyloxycarbonyl, vinyloxycarbonyl, β-(p-toluenesulfonyl)ethoxycarbonyl, and the like. Suitable amino acid residues include, but are not limited to, residues of Gly (glycine), Ala (alanine), Arg (arginine), Asn (asparagine), Asp (aspartic acid), Cys (cysteine), Glu (glutamic acid), His (histidine), lie (isoleucine), Leu (leucine), Lys (lysine), Met (methionine), Phe (phenylalanine), Pro (proline), Ser (serine), Thr (threonine), Trp (tryptophan), Tyr (tyrosine), Val (valine), Nva (norvaline), Hse (homoserine), 4-Hyp (4-hydroxyproline), 5 Hyl (5-hydroxylysine), Orn (ornithine) and R-Ala. Examples of suitable amino acid residues also include amino acid residues that are protected with a protecting group. Examples of suitable protecting groups include those typically employed in peptide synthesis, including acyl groups (such as formyl and acetyl), arylmethoxycarbonyl groups (such as benzyloxycarbonyl and p-nitrobenzyloxycarbonyl), tert butoxycarbonyl groups (—C(O)OC(CH₃)₃, Boc), and the like. Suitable peptide residues include peptide residues comprising two to five amino acid residues. The residues of these amino acids or peptides can be present in stereochemical configurations of the D-form, the L-form or mixtures thereof. In addition, the amino acid or peptide residue may have an asymmetric carbon atom. Examples of suitable amino acid residues having an asymmetric carbon atom include residues of Ala, Leu, Phe, Trp, Nva, Val, Met, Ser, Lys, Thr and Tyr. Peptide residues having an asymmetric carbon atom include peptide residues having one or more constituent amino acid residues having an asymmetric carbon atom. Examples of suitable amino acid protecting groups include those typically employed in peptide synthesis, including acyl groups (such as formyl and acetyl), arylmethoxycarbonyl groups (such as benzyloxycarbonyl and p nitrobenzyloxycarbonyl), tert-butoxycarbonyl groups (—C(O)OC(CH3)3), and the like. Other examples of substituents “convertible to hydrogen in vivo” include reductively eliminable hydrogenolyzable groups. Examples of suitable reductively eliminable hydrogenolyzable groups include, but are not limited to, arylsulfonyl groups (such as o-toluenesulfonyl); methyl groups substituted with phenyl or benzyloxy (such as benzyl, trityl and benzyloxymethyl); arylmethoxycarbonyl groups (such as benzyloxycarbonyl and o-methoxy-benzyloxycarbonyl); and haloethoxycarbonyl groups (such as β,β,β-trichloroethoxycarbonyl and β iodoethoxycarbonyl).

Compounds of the disclosure may also have the advantage that they may be more efficacious than, be less toxic than, be longer acting than, be more potent than, produce fewer side effects than, be more easily absorbed than, and/or have a better pharmacokinetic profile (e.g., higher oral bioavailability and/or lower clearance) than, and/or have other useful pharmacological, physical, or chemical properties over, compounds known in the prior art, whether for use in the indications stated herein or otherwise.

In some aspects, the present disclosure relates to compositions (e.g. pharmaceutical compositions) containing one or more VLA-4 inhibitors and one or more agents which interact with a chemokine receptor, such as a CXCR2 agonist or a CXCR4 inhibitor. Alternatively, these compositions may further comprise G-CSF. These compositions may further comprise an excipient such as solvent or diluent which renders the composition suitable for administration via injection. In some embodiments, the pharmaceutically active components of these compositions may be formulated independently and then administered simultaneously to a patient. In other embodiments, these compositions are formulated with additional therapeutic agents or excipients. In other embodiments, these compositions consist substantially of, consist essentially of, or consist of one or more VLA-4 inhibitors, one or more agents which interact with a chemokine, and one or more excipients. Each of the compositions described herein contain a pharmaceutically effective amount of each of these agents combined. In particular, the compositions may contain an pharmaceutically effective combined amount of the VLA-4 inhibitor and the agent which interacts with a chemokine receptor. The pharmaceutically effective combined amount results when each agent is present in an amount such that the effect of the combination results in increased activity relative to a similar amount of a single agent. In some embodiments, the effect of the combination results in an additive increase in activity. In some embodiments, the effect of the combination results in synergistic activity.

These compositions may be used in a variety of indications such as the mobilization of hematopoietic stem cells or progenitor cells. These indications include elevating the number of progenitor and/or stem cells which are circulating in the patient especially elevating the number of these cells in the peripheral blood of a patient. Alternatively, these compositions may be used to treat a patient with cancer including sensitizing the patient to a chemotherapy and/or radiotherapy, for the treatment of hematopoietic cancer such as leukemias, myelomas, or lymphoma, or for the harvesting of hematopoietic progenitor and/or stem cells which may be transplanted into a patient who has impaired production of hematopoietic progenitor and/or stem cells. A patient may have impaired production of hematopoietic progenitor and/or stem cells resulting from a high dose of chemotherapy, radiotherapy, another therapeutic agent, or a genetic abnormality. Alternatively, the compositions described herein may be used to mobilize pre-cancerous or cancerous cells from the bone marrow into the peripheral blood. In some embodiments, the mobilization of pre-cancerous or cancerous cells from the bone marrow is used to potentiate or increase the effects of a standard cancer therapy such as a chemotherapeutic and/or radiotherapy. Furthermore, each of these compositions may be used in the manufacture of medicament for these indications.

The compositions and methods described herein may include one or more additional agents that are therapeutically or nutritionally useful such as antibiotics, vitamins, herbal extracts, anti-inflammatories, glucose, antipyretics, analgesics, cyclophosphamide, recombinant stem cell factor (Stemgen®), granulocyte-macrophage colony stimulating factor (GM-CSF) (such as Leukine®, and Leucomax®), ETRX-101, TLK 199/TILENTRA™, Interleukin-1 (IL-1), Interleukin-3 (IL-3), Interleukin-8 (IL-8), PIXY-321 (GM-CSF/IL-3 fusion protein), macrophage inflammatory protein, thrombopoietin, or a similar agent. Additionally, the compositions may contain one or more agents prevents microbial growth to increase the storage of the composition. Such agents may be an anti-parasitic, an antifungal, an antibiotic, or anti-viral. Additionally, the compositions may further comprise one or more chemotherapeutic agents

(i)(a) VLA-4 Inhibitors

The methods and compositions used herein may contain one or more VLA-4 inhibitors or VLA-4 antagonists. The terms VLA-4 inhibitor and VLA-4 antagonist are used interchangeably in the disclosure. Some non-limiting examples of VLA-4 inhibitors which may be used in the compositions and methods described herein include antibodies, such as humanized monoclonal antibody against α4, natalizumab (Antegren®) and small molecules such as those described in U.S. Pat. No. 5,510,332; WO 06/023396; WO 97/03094; WO 97/02289; WO 96/40781; WO 96/22966; WO 96/20216; WO 96/01644; WO 96/06108; WO 95/15973; WO 96/31206; WO 06/010054; WO 05/087760; WO 01/12186; WO 99/37605; WO 01/51487; WO 03/011288; WO 02/14272; WO 01/32610; and EP 0842943, the entire contents of which are hereby incorporated by reference. An example of a VLA-4 inhibitor that may be used herein is BIO5192 (also known as AMD15057) disclosed in PCT publication WO 01/12186, which is incorporated herein by reference. Alternatively, analogs of BIO5192, such as BIO1211, may be used. In other embodiments, the VLA-4 inhibitor is firategrast or a pharmaceutically acceptable salt thereof. Firategrast is a compound of the formula:

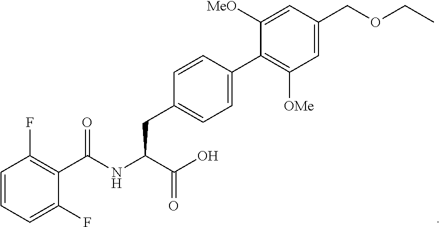 [SMILES: CCOCc1cc(OC)c(-c2ccc(C[C@H](NC(=O)c3c(F)cccc3F)C(=O)O)cc2)c(OC)c1]

In another aspect, the compositions or methods described herein may contain one or more VLA-4 inhibitors with a structure shown below:

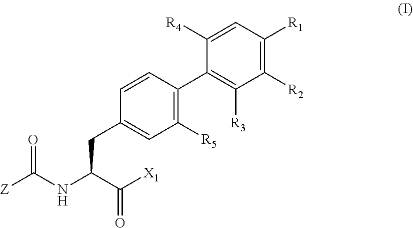

wherein:
R₁ is hydrogen, aminocarbonyl, carboxy, cyano, halo, hydroxy, —SF₅, alkyl_{(C≤8)}, substituted alkyl_{(C≤8)}, alkoxy_{(C≤8)}, substituted alkoxy_{(C≤8)}, or —Y₁—Rₐ; wherein:
Y₁ is alkanediyl_{(C≤8)} or substituted alkanediyl_{(C≤8)}; and
Rₐ is alkoxy_{(C≤12)}, acyloxy_{(C≤12)}, or a substituted version of either of these groups; or
—X(CH₂O)ₘ—(CH₂CH₂O)ₙ—R_{b}; wherein:
X is a covalent bond or —O—;
m is 0 or 1;
n is 1, 2, 3, 4, 5, 6, 7, or 8; and
R_{b} is hydrogen, alkyl_{(C≤8)}, or substituted alkyl_{(C≤8)}; or
—CH₂—O—(CH₂)ₙ—R_{c}; wherein:
N is 0, 1, or 2; and
R_{c} is a cyclic aliphatic ring of 4-6 in size having one oxygen as part of the ring;
R₂ is hydrogen, aminocarbonyl, carboxy, cyano, halo, hydroxy, —SF₅, alkyl_{(C≤8)}, substituted alkyl_{(C≤8)}, alkoxy_{(C≤8)}, substituted alkoxy_{(C≤8)}, or —Y₂—R_{c}; wherein:
Y₂ is alkanediyl_{(C≤8)} or substituted alkanediyl_{(C≤8)}; and
R_{c} is alkoxy_{(C≤12)}, acyloxy_{(C≤12)}, or a substituted version of either of these groups;
X₁ is hydroxy, alkoxy_{(C≤8)}, substituted alkoxy_{(C≤8)}, cycloalkoxy_{(C≤8)}, substituted cycloalkoxy_{(C≤8)}, alkenyloxy_{(C≤8)}, substituted alkenyloxy_{(C≤8)}, aryloxy_{(C≤8)}, substituted aryloxy_{(C≤8)}, aralkoxy_{(C≤8)}, substituted aralkoxy_{(C≤8)}, or a substituent convertible in vivo to hydroxy; and
R₃ and R₄ are each independently hydrogen, hydroxy, alkoxy_{(C≤8)} or substituted alkoxy_{(C≤8)};
R₅ is hydrogen, —CH(OR_{d})Rₑ, or —C(O)R_{f}, wherein:
R_{d} is hydrogen, alkyl_{(C≤8)}, or substituted alkyl_{(C≤8)}; and
Rₑ and R_{f} are each independently alkyl_{(C≤8)} or substituted alkyl_{(C≤8)}; and
Z is a group of the formula:

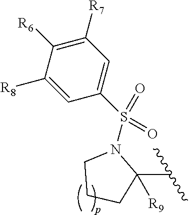

wherein:
p is 0, 1, 2, or 3;
R₆ is hydrogen or —C(O)X₂; wherein:
X₂ is amino, hydroxy, alkoxy_{(C≤8)}, substituted alkoxy_{(C≤8)}, cycloalkoxy_{(C≤8)}, substituted cycloalkoxy_{(C≤8)}, alkenyloxy_{(C≤8)}, substituted alkenyloxy_{(C≤8)}, aryloxy_{(C≤8)}, substituted aryloxy_{(C≤8)}, aralkyloxy_{(C≤8)}, substituted aralkyloxy_{(C≤8)}, alkylamino_{(C≤8)}, substituted alkylamino_{(C≤8)}, dialkylamino_{(C≤8)}, substituted dialkylamino_{(C≤8)}, cycloalkylamino_{(C≤8)}, substituted cycloalkylamino_{(C≤8)}, alkenylamino_{(C≤8)}, substituted alkenylamino_{(C≤8)}, arylamino_{(C≤8)}, substituted arylamino_{(C≤8)}, aralkylamino_{(C≤8)}, substituted aralkylamino_{(C≤8)}, or a substituent convertible in vivo to hydroxy;
R₇ and R₈ are each independently hydrogen, halo, haloalkyl_{(C≤8)}, or substituted haloalkyl_{(C≤8)};
R₉ is hydrogen, alkyl_{(C≤8)}, or substituted alkyl_{(C≤8)}; or
a group of the formula:

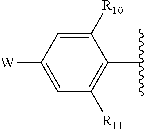

wherein:
W is hydrogen, cyano, halo, hydroxy, or —C(O)X₃, wherein:
X₃ is amino, hydroxy, alkoxy_{(C≤8)}, substituted alkoxy_{(C≤8)}, cycloalkoxy_{(C≤8)}, substituted cycloalkoxy_{(C≤8)}, alkenyloxy_{(C≤8)}, substituted alkenyloxy_{(C≤8)}, aryloxy_{(C≤8)}, substituted aryloxy_{(C≤8)}, aralkyloxy_{(C≤8)}, substituted aralkyloxy_{(C≤8)}, alkylamino_{(C≤8)}, substituted alkylamino_{(C≤8)}, dialkylamino_{(C≤8)}, substituted dialkylamino_{(C≤8)}, cycloalkylamino_{(C≤8)}, substituted cycloalkylamino_{(C≤8)}, alkenylamino_{(C≤8)}, substituted alkenylamino_{(C≤8)}, arylamino_{(C≤8)}, substituted arylamino_{(C≤8)}, aralkylamino_{(C≤8)}, substituted aralkylamino_{(C≤8)}, or a substituent convertible in vivo to hydroxy;
R₁₀ and R₁₁ are each independently hydrogen or halo;
or a pharmaceutically acceptable salt thereof.

TABLE 1
Exemplary VLA-4 Inhibitors

| Example Nos. | Structure |
|---|---|
| BIO5192 | 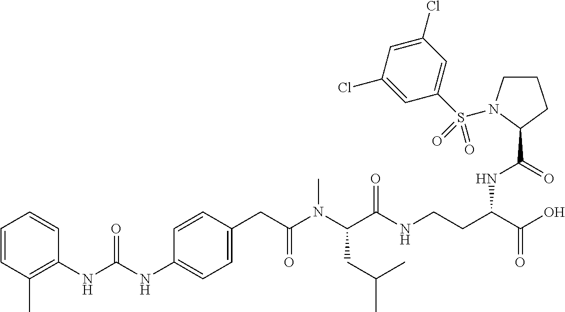 [SMILES: Cc1ccccc1NC(=O)Nc1ccc(CC(=O)N(C)[C@@H](CC(C)C)C(=O)NCC[C@H](NC(=O)[C@@H]2CCCN2S(=O)(=O)c2cc(Cl)cc(Cl)c2)C(=O)O)cc1] |
| Firategrast | 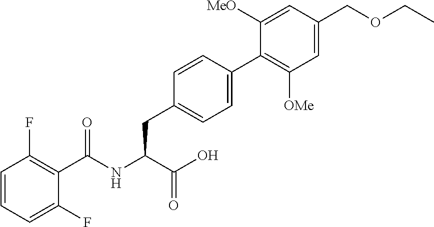 [SMILES: CCOCc1cc(OC)c(-c2ccc(C[C@H](NC(=O)c3c(F)cccc3F)C(=O)O)cc2)c(OC)c1] |
| 822 | 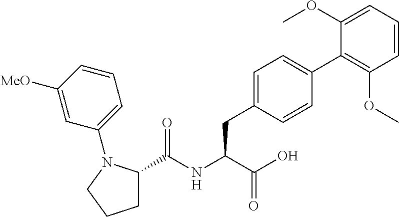 [SMILES: COc1cccc(N2CCC[C@H]2C(=O)N[C@@H](Cc2ccc(-c3c(OC)cccc3OC)cc2)C(=O)O)c1] |
| 823 | 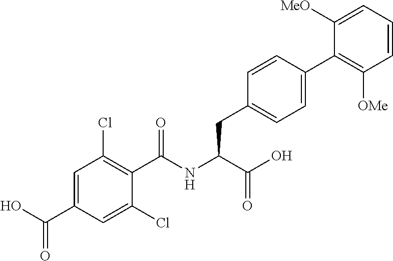 [SMILES: COc1cccc(OC)c1-c1ccc(C[C@H](NC(=O)c2c(Cl)cc(C(=O)O)cc2Cl)C(=O)O)cc1] |
| 824 | 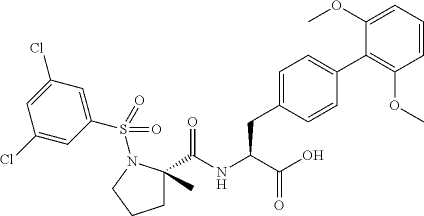 [SMILES: COc1cccc(OC)c1-c1ccc(C[C@H](NC(=O)[C@]2(C)CCCN2S(=O)(=O)c2cc(Cl)cc(Cl)c2)C(=O)O)cc1] |
| 825 | 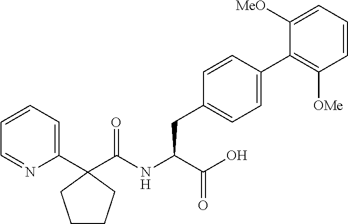 [SMILES: COc1cccc(OC)c1-c1ccc(C[C@H](NC(=O)C2(c3ccccn3)CCCC2)C(=O)O)cc1] |
| 842 | 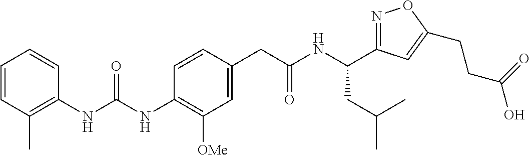 [SMILES: COc1cc(CC(=O)N[C@@H](CC(C)C)c2cc(CCC(=O)O)on2)ccc1NC(=O)Nc1ccccc1C] |
| 1173 | 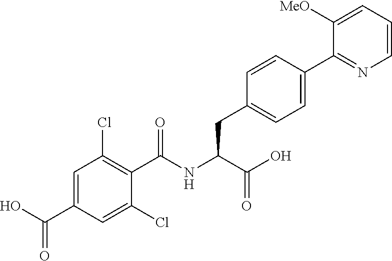 [SMILES: COc1cccnc1-c1ccc(C[C@H](NC(=O)c2c(Cl)cc(C(=O)O)cc2Cl)C(=O)O)cc1] |
| 1174 | 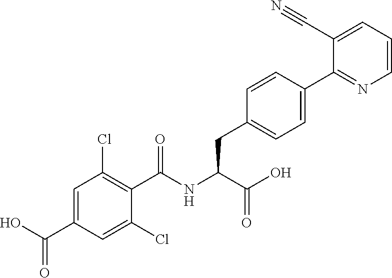 [SMILES: N#Cc1cccnc1-c1ccc(C[C@H](NC(=O)c2c(Cl)cc(C(=O)O)cc2Cl)C(=O)O)cc1] |
| 1219 | 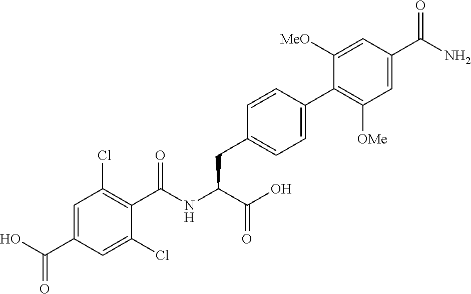 [SMILES: COc1cc(C(N)=O)cc(OC)c1-c1ccc(C[C@H](NC(=O)c2c(Cl)cc(C(=O)O)cc2Cl)C(=O)O)cc1] |
| 1220 | 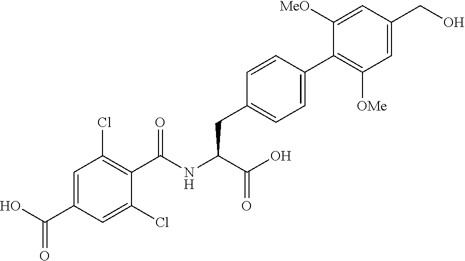 [SMILES: COc1cc(CO)cc(OC)c1-c1ccc(C[C@H](NC(=O)c2c(Cl)cc(C(=O)O)cc2Cl)C(=O)O)cc1] |
| 1221 | 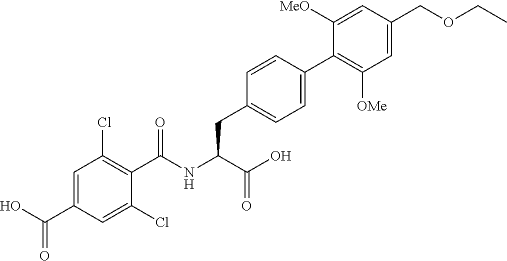 [SMILES: CCOCc1cc(OC)c(-c2ccc(C[C@H](NC(=O)c3c(Cl)cc(C(=O)O)cc3Cl)C(=O)O)cc2)c(OC)c1] |
| 1222 | 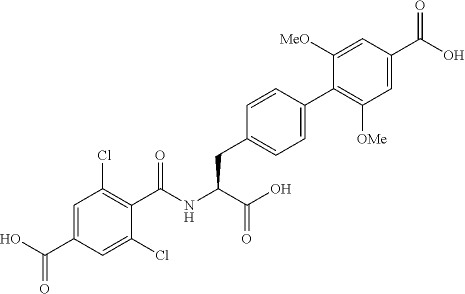 [SMILES: COc1cc(C(=O)O)cc(OC)c1-c1ccc(C[C@H](NC(=O)c2c(Cl)cc(C(=O)O)cc2Cl)C(=O)O)cc1] |
| 1223 | 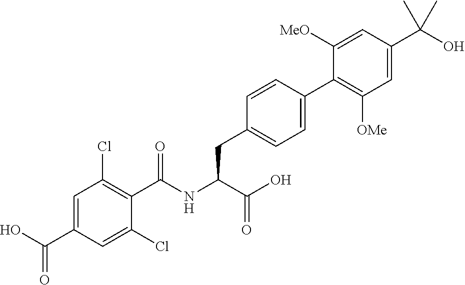 [SMILES: COc1cc(C(C)(C)O)cc(OC)c1-c1ccc(C[C@H](NC(=O)c2c(Cl)cc(C(=O)O)cc2Cl)C(=O)O)cc1] |
| 1224 | 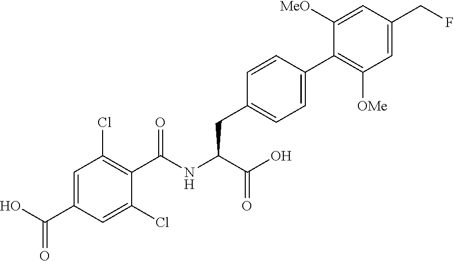 [SMILES: COc1cc(CF)cc(OC)c1-c1ccc(C[C@H](NC(=O)c2c(Cl)cc(C(=O)O)cc2Cl)C(=O)O)cc1] |
| 1512 | 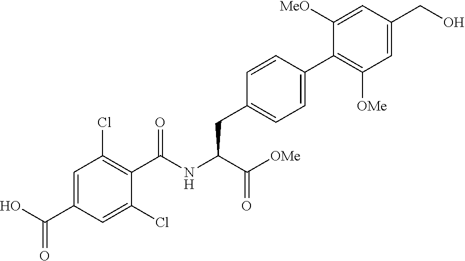 [SMILES: COC(=O)[C@H](Cc1ccc(-c2c(OC)cc(CO)cc2OC)cc1)NC(=O)c1c(Cl)cc(C(=O)O)cc1Cl] |
| 1554 | 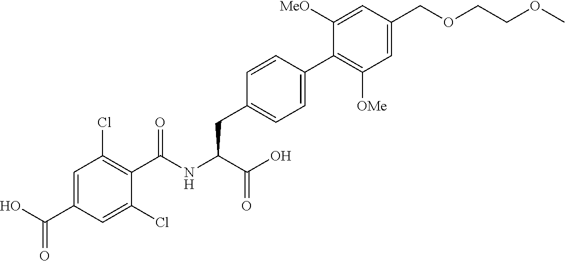 [SMILES: COCCOCc1cc(OC)c(-c2ccc(C[C@H](NC(=O)c3c(Cl)cc(C(=O)O)cc3Cl)C(=O)O)cc2)c(OC)c1] |
| 1555 | 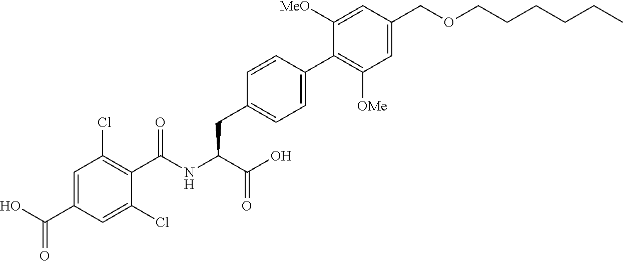 [SMILES: CCCCCCOCc1cc(OC)c(-c2ccc(C[C@H](NC(=O)c3c(Cl)cc(C(=O)O)cc3Cl)C(=O)O)cc2)c(OC)c1] |
| 1570 | 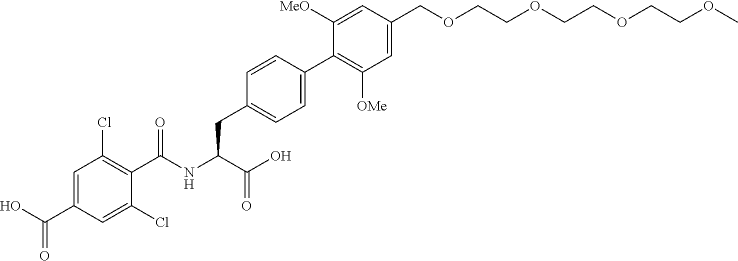 [SMILES: COCCOCCOCCOCc1cc(OC)c(-c2ccc(C[C@H](NC(=O)c3c(Cl)cc(C(=O)O)cc3Cl)C(=O)O)cc2)c(OC)c1] |
| 1571 | 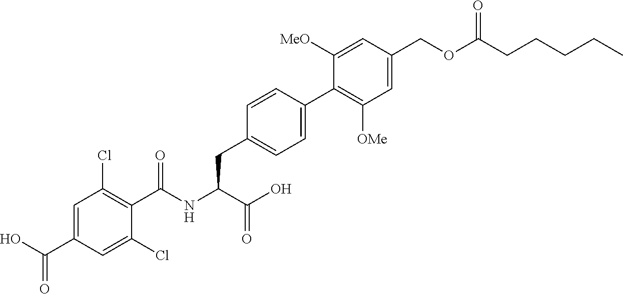 [SMILES: CCCCCC(=O)OCc1cc(OC)c(-c2ccc(C[C@H](NC(=O)c3c(Cl)cc(C(=O)O)cc3Cl)C(=O)O)cc2)c(OC)c1] |
| 1608 | 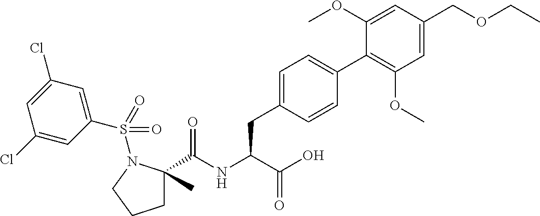 [SMILES: CCOCc1cc(OC)c(-c2ccc(C[C@H](NC(=O)[C@]3(C)CCCN3S(=O)(=O)c3cc(Cl)cc(Cl)c3)C(=O)O)cc2)c(OC)c1] |
| 1609 | 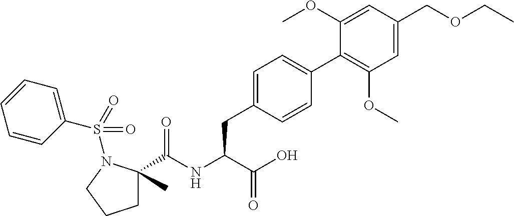 [SMILES: CCOCc1cc(OC)c(-c2ccc(C[C@H](NC(=O)[C@]3(C)CCCN3S(=O)(=O)c3ccccc3)C(=O)O)cc2)c(OC)c1] |
| 1610 | 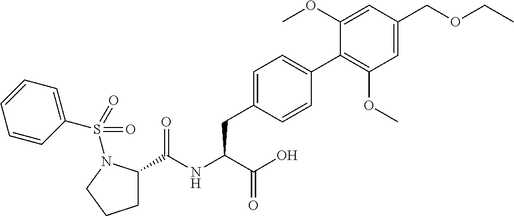 [SMILES: CCOCc1cc(OC)c(-c2ccc(C[C@H](NC(=O)[C@@H]3CCCN3S(=O)(=O)c3ccccc3)C(=O)O)cc2)c(OC)c1] |
| 1611 | 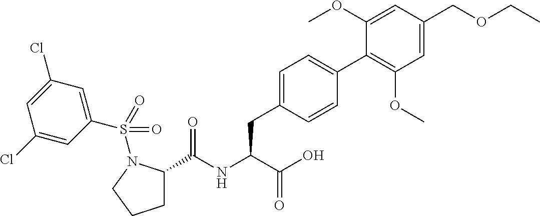 [SMILES: CCOCc1cc(OC)c(-c2ccc(C[C@H](NC(=O)[C@@H]3CCCN3S(=O)(=O)c3cc(Cl)cc(Cl)c3)C(=O)O)cc2)c(OC)c1] |
| 1632 | 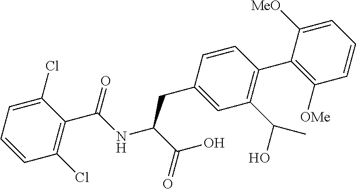 [SMILES: COc1cccc(OC)c1-c1ccc(C[C@H](NC(=O)c2c(Cl)cccc2Cl)C(=O)O)cc1C(C)O] |
| 1633 | 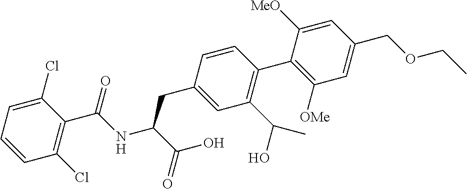 [SMILES: CCOCc1cc(OC)c(-c2ccc(C[C@H](NC(=O)c3c(Cl)cccc3Cl)C(=O)O)cc2C(C)O)c(OC)c1] |
| 1745 | 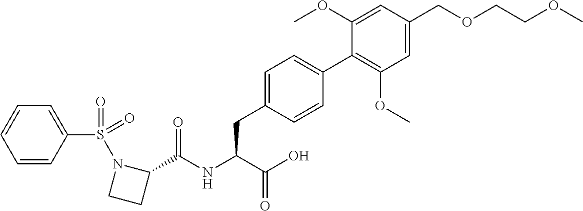 [SMILES: COCCOCc1cc(OC)c(-c2ccc(C[C@H](NC(=O)[C@@H]3CCN3S(=O)(=O)c3ccccc3)C(=O)O)cc2)c(OC)c1] |
| 1746 | 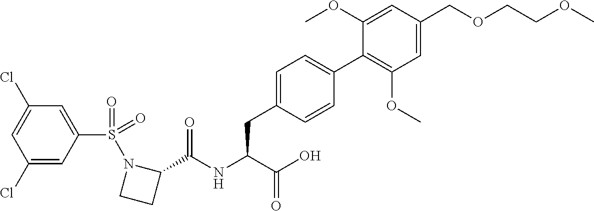 [SMILES: COCCOCc1cc(OC)c(-c2ccc(C[C@H](NC(=O)[C@@H]3CCN3S(=O)(=O)c3cc(Cl)cc(Cl)c3)C(=O)O)cc2)c(OC)c1] |
| 1747 | 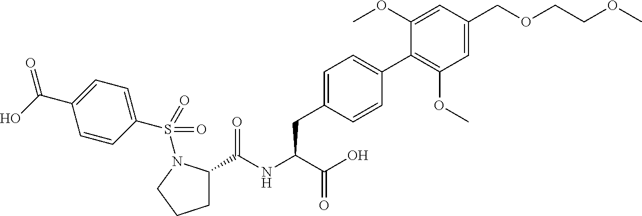 [SMILES: COCCOCc1cc(OC)c(-c2ccc(C[C@H](NC(=O)[C@@H]3CCCN3S(=O)(=O)c3ccc(C(=O)O)cc3)C(=O)O)cc2)c(OC)c1] |
| 2087 | 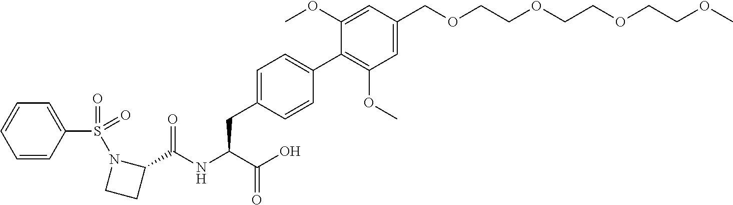 [SMILES: COCCOCCOCCOCc1cc(OC)c(-c2ccc(C[C@H](NC(=O)[C@@H]3CCN3S(=O)(=O)c3ccccc3)C(=O)O)cc2)c(OC)c1] |
| 2088 | 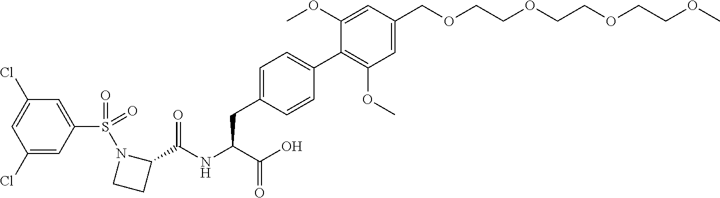 [SMILES: COCCOCCOCCOCc1cc(OC)c(-c2ccc(C[C@H](NC(=O)[C@@H]3CCN3S(=O)(=O)c3cc(Cl)cc(Cl)c3)C(=O)O)cc2)c(OC)c1] |
| 009 | 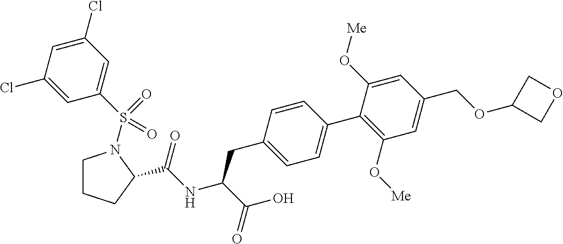 [SMILES: COc1cc(COC2COC2)cc(OC)c1-c1ccc(C[C@H](NC(=O)[C@@H]2CCCN2S(=O)(=O)c2cc(Cl)cc(Cl)c2)C(=O)O)cc1] |

(i)(b) an Agent which Interacts with a Chemokine Receptor

As used herein, the “agent which interacts with a chemokine receptor” includes chemokines, cytokines, chemokine receptors, or an agent which modulates the activity of these molecules such as a fragment, an antibody, or a small organic molecule. In one embodiment, the present disclosure relates to compositions which modulate the activity of a CXC chemokine receptor such as CXCR2 or CXCR4. In some embodiments, the present methods and compositions contain at least one CXCR2 agonists.

CXCR2 agonists include any molecule that activates the CXCR2 receptor. Such molecules include chemokines, cytokines, agonist antibodies or biologically active fragments thereof, or small organic molecules. Some non-limiting examples of chemokines acting via the CXCR2 receptor include, but are not limited to Groβ, Groα, Groγ, GCP-2 (granulocyte chemo-attractant protein 2), IL-8, NAP-2 (neutrophil activating peptide 2), ENA-78 (epithelial-cell derived neutrophil activating protein 78), and MGSA.

In some embodiments, the CXCR2 agonists used in the methods and compositions described herein are Groβ and modified forms thereof. King et al., 2001 have demonstrated that a recombinant N-terminal 4-amino acid truncated form of the human chemokine Groβ (also known as SB-251353 or garnocestim or Groβt or tGro-β) can mobilize progenitor cells after administration of SB-251353 in combination with G-CSF. This combination resulted in the mobilization of neutrophils and platelets during these studies. Chemokines such as the SB-251353, Groα, Groβ, and Groγ are further discussed in WO 94/29341; WO 97/15594; WO 97/15595; WO 99/26645; WO 02/02132; U.S. Pat. Nos. 6,080,398; 6,399,053; and 6,447,766, which are incorporated herein by reference.

The “Groβ”, “Groβ protein”, or “Groβ chemokine” class includes Groβ itself as well as modified forms of Groβ. These modified forms include, but are not limited to, truncated, multimerized, amino-acid substituted, modified with amino-acid deletions and/or insertions, or combinations thereof. “Modified forms of Groβ” includes truncated forms such as those described in U.S. Pat. Nos. 6,447,766; 6,399,053; 6,080,398; PCT publication 99/26645; PCT publication WO 97/15595; PCT publication WO 02/02132; PCT publication WO 97/15594; and PCT publication WO 94/29341, which are incorporated herein by reference. “Modified forms of Groβ” are multimeric forms of Groβ such as dimers, trimers, tetramers, or other versions containing multiple proteins or modified proteins. Some non-limiting examples of “modified forms” include modified forms of Groβ with truncation of between 2 to about 8 amino acids at the amino terminus of the mature protein, truncation of between about 2 to about 10 amino acids at the carboxy terminus of the mature protein, or multimeric forms of the modified and/or truncated proteins, e.g., dimers, trimers, tetramers and other aggregated forms. Some non-limiting examples of truncated forms of Groβ may include SB-251353 which consists of amino acids 5-73 and forms thereof where amino acid 69 is deamidated.

Another specific CXCR2 receptor agonist that may be used in the compositions and methods described herein is SB-251353, a basic, heparin-binding protein with a molecular mass of approximately 7500 Da (King et al., 2000, Hepburn et al., 2001).

The compositions and methods described herein may comprise one or more CXCR4 inhibitors (e.g. antagonists). Some non-limiting examples of CXCR4 inhibitors include AMD3100 (plerixafor), AMD3465, CTCE-0214, CTCE-9908, CP-1221 (linear peptides, cyclic peptides, natural amino-acids, unnatural amino acids, and peptidomimetic compounds), T140 and analogs, 4F-benzoyl-TN24003, KRH-1120, KRH-1636, KRH-2731, polyphemusin analogue, ALX40-4C, or a CXCR4 inhibitors described in WO 01/85196, WO 99/50461, WO 01/94420, WO 03/090512, US 2005/0059702, US 2005027767, US 2003/9229341, U.S. Pat. Nos. 5,021,409, 6,001,826, and 5,583,131, each of which is incorporated by reference herein.

The compositions or methods described herein may comprise G-CSF. It is contemplated that any suitable source of G-CSF may be employed. The G-CSF used in the compositions or methods may be either recombinant or purified using known techniques and includes, but is not limited to, Neupogen®/filgrastim (Amgen), Neutrogin®/Granocyte®/lenograstim (Chugai Pharmaceuticals), and Neulasta® pegylated filgrastim (Amgen). Additionally, biologically active fragments, variants, derivatives or fusion proteins may also be employed provided these agents retain the ability to mobilize progenitor or stem cells.

(ii) Biological Activity

It is another object of the disclosure to provide pharmaceutical compositions comprising compounds described above. These compounds and pharmaceutical compositions may be used to improve the harvest of hematopoietic stem cells or progenitor cells. Additionally, the compounds or compositions may be used to elevate the circulation of hematopoietic progenitor and/or stem cells, improve the collection of hematopoietic stem cells or progenitor cells for a transfusion, increase the sensitization of an anti-cancer therapy such as a chemotherapeutic or radiotherapy, or mobilize pre-cancerous or cancerous cells into the peripheral blood which may increase their sensitivity to an anti-cancer therapy.

Hematopoietic stem cell transplant (HSCT) is used to facilitate repopulation of healthy bone marrow and immune system cells after high-dose chemotherapy treatment for cancers such as Hodgkin's and non-Hodgkin's lymphoma, multiple myeloma, and leukemia. In HSCT, hematopoietic stem/progenitor cells (HSPCs) are collected from the patient's blood, harvested, frozen and then stored while the patient receives high-dose chemotherapy and/or radiation therapy. Successful HSCT requires the intravenous infusion of a minimum number of 2×10⁶ CD34+ stem cells/kg body weight; however, a dose of 5×10⁶ CD34+ cells/kg is considered preferable for early and long term multilineage engraftment.

Stem cells harvested from peripheral blood are the most commonly used graft source in HSCT. While granulocyte colony-stimulating factor (G-CSF) is the most frequently used agent for stem cell mobilization, the use of G-CSF alone results in suboptimal stem cell yields in a significant proportion of patients. Plerixafor (AMD3100), a small molecule CXCR4 antagonist, in combination with G-CSF increases total CD34+ HSPCs compared to G-CSF alone and is FDA approved for stem cell mobilization in Non-Hodgkin's lymphoma and multiple myeloma. However, a significant disadvantage of plerixafor is cost, adding $25,567 per patient compared to G-CSF alone. Furthermore, up to 24% of patients receiving plerixafor and G-CSF still fail to collect ≥2×10⁶ CD34+ cells/kg in 4 days of apheresis. Recent economic analysis has determined that reducing apheresis by 1 day has the potential to decrease medical costs by $6,600. Thus improved/alternative mobilizing agents and strategies are needed.

Mechanistic studies have shown that the integrin α4β1 (VLA-4) plays an important role in the retention of HSPCs within the bone marrow (BM) microenvironment. HSPC mobilization has been achieved by disrupting the integrin α4β1/VCAM-1 axis with antibodies against integrin α4β1 or VCAM-1. Preclinical mouse studies in the DiPersio laboratory have shown that administration of the small molecule inhibitor of integrin α4β1, BIO5192, results in the rapid and reversible mobilization of HSPCs into the peripheral circulation with maximum mobilization occurring within 30 to 60 minutes and returning to baseline within 4 hours. A superior treatment could be envisioned wherein a patient receives an integrin α4β1 antagonist to continually inhibit integrin α4β1 over the course of ˜4 hours (average duration of CD34+ stem cell apheresis procedures), maximizing the mobilization of HSPCs that can be collected by apheresis during the same day of treatment.

BIO5192 is a potent small molecule inhibitor of integrin α4β1 and has demonstrated efficacy in mobilizing HSPC's in mice. However, BIO5192 has poor aqueous solubility, bioavailability, and pharmacokinetic properties and therefore has not been developed clinically. A simpler, more soluble integrin α4β1 antagonist is firategrast. Firategrast has been tested in clinical trials for the treatment of multiple sclerosis and has demonstrated efficacy in mobilizing HSPC's in mice but to a significantly lesser extent than BIO5192 and at much higher doses. Thus, development of integrin α4β1 antagonist which exhibit improved properties would greatly improve the clinical use of this particular combination.

Integrin antagonist compounds with high VLA-4 binding affinity and in vivo efficacy similar to BIO5192 but with superior pharmacokinetic and physiochemical properties are expected to increase mobilization of HSPCs alone or in a synergistic manner with G-CSF, plerixafor, truncated Gro-beta (Groβt) or other mobilization agents and thus remarkably improve the current HSCT regimen. These compositions or methods may also have the added advantage that the compositions or methods result in the mobilization in higher numbers, begin mobilization in a shorter period of time, over a more prolonged period of time, or mobilize increased numbers of early progenitor and/or stem cells, LSK-SLAM cells, CFU-C cells, or other progenitor and/or stem cells which are competent to achieve a successful engraftment into the patient. For example, the number of progenitor and/or stem cells mobilized when using the combination or methods described herein may be at least about 1.2-fold, at least about 1.5-fold, at least about 2-fold, at least about 2.5-fold, at least about 3-fold, at least about 3.5-fold, at least about 4-fold, at least about 4.5-fold, at least about 5-fold, at least about 5.5-fold, at least about 6-fold, at least about 6.5-fold, at least about 7-fold, at least about 7.5-fold, at least about 8-fold, at least about 8.5-fold, at least about 9-fold, at least about 9.5-fold, at least about 10-fold, at least about 11-fold, at least about 12-fold, at least about 13-fold, at least about 14-fold, or at least about 15-fold greater then when using a single agent alone. Specifically, the number of early progenitor and/or stem cells (e.g. LSK-SLAM cells) mobilized when using the combination of at least one VLA-4 inhibitor and at least one CXCR2 agonist is at least about 1.2-fold, at least about 1.5-fold, at least about 2-fold, at least about 2.5-fold, at least about 3-fold, at least about 3.5-fold, at least about 4-fold, at least about 4.5-fold, at least about 5-fold, at least about 5.5-fold, at least about 6-fold, at least about 6.5-fold, at least about 7-fold, at least about 7.5-fold, at least about 8-fold, at least about 8.5-fold, at least about 9-fold, at least about 9.5-fold, at least about 10-fold, at least about 11-fold, at least about 12-fold, at least about 13-fold, at least about 14-fold, at least about 15-fold, at least about 16-fold, at least about 17-fold, at least about 18-fold, at least about 19-fold, at least about 20-fold, at least about 21-fold, at least about 22-fold, at least about 23-fold, at least about 24-fold, or at least about 25-fold greater then when using a single agent alone.

(iii) Therapeutic Methods

In one aspect, this disclosure provides methods of inhibiting or antagonizing VLA-4 using one or more of the compounds disclosed herein, as well as pharmaceutical compositions thereof containing one or more VLA-4 antagonists in the presence of one or more agents such as Groβ, G-CSF, or a derivative thereof. The therapeutic methods described herein may be used to enhance or elevate the circulation of hematopoietic progenitor and/or stem cells. These therapeutic methods may be used to improve stem cell transplantation, tissue repair, improve the efficacy of cancer therapy, or other situations in which in vivo stimulation of hematopoiesis is desirable. The compositions or methods described herein wherein the VLA-4 inhibitor and agent which interacts with a chemokine receptor combine to act synergistically to induce rapid mobilization of progenitor and stem cells. For example, peak mobilization, when these combined therapeutic agents are used, may occur at about 10 minutes, about 15 minutes, about 20 minutes, about 25 minutes, about 30 minutes, about 45 minutes, about 1 hour, about 2 hours, about 3 hours, about 4 hours, about 5 hours, or about 6 hours after administration of the combination. In one embodiment, these compositions or methods result in a composition which acts synergistically to induce rapid mobilization of progenitor and stem cells with peak mobilization at about 15 minutes after administration of the combination. In contrast, this mobilization is significantly shorter than the 4-5 days needed to achieve maximum mobilization using G-CSF.

In one aspect, the methods and compositions described herein may be used to increase the harvest of HSPCs for a variety of different applications. These compositions and methods may be used to treat a patient who requires a transplantation. Alternatively, the compositions and methods may be used to treat a patient who does not require a transplantation. The patient who needs a transplant of HSPCs requires either an allogenic, autologous, or tandem transplant of HSPCs. In some embodiments, the HSPCs may be used in either allogenic or autologous transplants. In another aspect, the present methods and compositions described herein may be used to improve the circulation of cells to tissues which need repair. The increased circulation of HSPCs may be used to improve the repair of the target tissue in the patient.

If the HSPCs are harvested, these cells may be returned to the donor patient (autologous transplant) or may be donated to another patient that is sufficiently compatible to prevent rejection (allogeneic transplant). One non-limiting application of autologous transplantation is in combination with radiation or chemotherapy in patients bearing tumors since the radiotherapeutic or chemotherapeutic methods deplete the patient's normal cells. In this application, the patients cells may be harvested prior to or during the therapeutic treatments, fractionated if necessary, cultured and optionally expanded, and then returned to the patient to restore the damaged immune system depleted by the therapy. Allogeneic recipients may receive the cells for the same purpose, or may have a condition that may be benefited by enhancing their hematopoietic systems. In a typical protocol for these types of transplants, the mobilized cells are collected from the donor by, for example, apheresis and then stored/cultured/expanded/fractionated as desired. In some embodiments, the compositions or methods described herein may result in the need for apheresis being eliminated.

In some aspects, the present compositions and methods described herein may be used to increase the circulation of pre-cancerous or cancerous cells out of the bone marrow into the peripheral blood. Without wishing to be bound by any theory, it is believed that the increasing the circulation of pre-cancerous or cancerous cells out of the bone marrow may increase the effectiveness of an anti-cancer therapy. In particular, these methods or compositions may be used to treat patients who have or are at risk of a hematopoietic malignancy such as lymphoma, myeloma, or leukemia. The compositions or methods described herein may be administered or employed prior to, during, or subsequent to the anti-cancer therapy. Two non-limiting examples of anti-cancer therapies that may be used in the methods described herein or conjunction with the compositions described herein include chemotherapeutic agents or radiotherapy.

In another aspect, the compositions and methods described herein may be used to decrease inflammation which may result in increasing tissue repair. Thus, the compositions or methods described herein may be used to treat graft versus host disease. Additionally, these compositions or methods may be used to treat diseases or disorders associated with cell adhesion-mediated inflammatory pathways. Some non-limiting examples of cell adhesion-mediated inflammatory pathologies include asthma, multiple sclerosis, rheumatoid arthritis, atherosclerosis, and inflammatory bowel disease.

The pharmaceutical compositions of these combinations may further comprise one or more non-toxic, pharmaceutically acceptable carriers and/or diluents and/or adjuvants (collectively referred to herein as “carrier” materials) and if desired other active ingredients. These methods may be used to treat a blood disease or disorder such as sickle cell anemia or as a part of hematopoietic stem cell therapy to promote the development of stem cells. In some embodiments, the compound is administered as part of a pharmaceutical composition further comprising a pharmaceutically acceptable carrier. In some embodiments, the compounds and/or pharmaceutical compositions thereof may be administered orally, parenterally, or by inhalation spray, or topically in unit dosage formulations containing conventional pharmaceutically acceptable carriers, adjuvants and vehicles. The term parenteral as used herein includes, for example, subcutaneous, intravenous, intramuscular, intrasternal, infusion techniques or intraperitoneally. In some embodiments, the compounds of the present disclosure are administered by any suitable route in the form of a pharmaceutical composition adapted to such a route, and in a dose effective for the treatment intended. Therapeutically effective doses of the compounds required to prevent or arrest the progress of or to treat a medical condition are readily ascertained by one of ordinary skill in the art using preclinical and clinical approaches familiar to the medicinal arts.

Based upon standard laboratory experimental techniques and procedures well known and appreciated by those skilled in the art, as well as comparisons with compounds of known usefulness, the compounds described above can be used in the treatment of patients suffering from the above pathological conditions. One skilled in the art will recognize that selection of the most appropriate compound of the disclosure is within the ability of one with ordinary skill in the art and will depend on a variety of factors including assessment of results obtained in standard assay and animal models.

In several aspects of the present disclosure, the compounds provided herein may be used in a variety of biological, prophylactic or therapeutic areas, including those in wherein VLA-4 plays a role.

(iv) Pharmaceutical Formulations and Routes of Administration

For administration to an animal especially a mammal in need of such treatment, the compounds in a therapeutically effective amount are ordinarily combined with one or more excipients appropriate to the indicated route of administration. The compounds of the present disclosure are contemplated to be formulated in a manner amenable to treatment of a veterinary patient as well as a human patient. In some embodiments, the veterinary patient may be an avian such as chicken, turkey, or duck, a companion animal such as a cat or dog, livestock animals such as a cow, horse, pig, or goat, zoo animals, and wild animals The compounds may be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia, sodium alginate, polyvinylpyrrolidone, and/or polyvinyl alcohol, and tableted or encapsulated for convenient administration. Alternatively, the compounds may be dissolved in water, polyethylene glycol, propylene glycol, ethanol, corn oil, cottonseed oil, peanut oil, sesame oil, benzyl alcohol, sodium chloride, and/or various buffers. Other excipients and modes of administration are well and widely known in the pharmaceutical art and may be adapted to the type of animal being treated. Description of potential administration routes which may be used to formulate the compositions described herein include those taught in Remington's Pharmaceutical Sciences, latest edition, Mack Publishing Company, Easton, Pa., which is incorporated herein by reference.

The pharmaceutical compositions useful in the present disclosure may be subjected to conventional pharmaceutical operations such as sterilization and/or may contain conventional pharmaceutical carriers and excipients such as preservatives, stabilizers, wetting agents, emulsifiers, buffers, etc.

The compounds of the present disclosure may be administered by a variety of methods, e.g., orally or by injection (e.g. subcutaneous, intravenous, intraperitoneal, etc.). Depending on the route of administration, the active compounds may be coated in a material to protect the compound from the action of acids and other natural conditions which may inactivate the compound. They may also be administered by continuous perfusion/infusion of a disease or wound site.

In one aspect, the present methods or compositions may be administered such that the VLA-4 inhibitor is administered intravenously and the agent which interacts with a chemokine receptor is administered subcutaneously. Alternatively, the VLA-4 inhibitor and the agent which interacts with a chemokine receptor may be both administered subcutaneously. In still another embodiment, both the VLA-4 inhibitor and the agent which interacts with a chemokine receptor are administered subcutaneously in a single formulation. In some embodiments, the VLA-4 inhibitor and the agent which interacts with a chemokine receptor are administered as a single formulation subcutaneously or intravenously. The protocols for administration to a particular patient may be further optimized by a skilled practitioner.

To administer the therapeutic compound by other than parenteral administration, it may be necessary to coat the compound with, or co-administer the compound with, a material to prevent its inactivation. For example, the therapeutic compound may be administered to a patient in an appropriate carrier, for example, liposomes, or a diluent. Pharmaceutically acceptable diluents include saline and aqueous buffer solutions. Liposomes include water-in-oil-in-water CGF emulsions as well as conventional liposomes.

The therapeutic compound may also be administered parenterally, intraperitoneally, intramuscularly, intraarterially, intraspinally, or intracerebrally. Dispersions can be prepared in glycerol, liquid polyethylene glycols, and mixtures thereof and in oils. Under ordinary conditions of storage and use, these preparations may contain a preservative to prevent the growth of microorganisms.

Pharmaceutical compositions may be suitable for injectable use include sterile aqueous solutions (where water soluble) or dispersions and sterile powders for the extemporaneous preparation of sterile injectable solutions or dispersion. In all cases, the composition must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (such as, glycerol, propylene glycol, and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it may be useful to include isotonic agents, for example, sugars, sodium chloride, or polyalcohols such as mannitol and sorbitol, in the composition. Prolonged absorption of the injectable compositions can be brought about by including in the composition an agent which delays absorption, for example, aluminum monostearate or gelatin. For intravenous or parenteral administration, the compounds are formulated in suitable liquid form with excipients as required. The compositions may contain liposomes or other suitable carriers. For injection intravenously, the solution is made isotonic using standard preparations such as Hank's solution or other isotonic solutions.

Sterile injectable solutions can be prepared by incorporating the therapeutic compound in the required amount in an appropriate solvent with one or a combination of ingredients enumerated above, as required, followed by filtered sterilization. Generally, dispersions are prepared by incorporating the therapeutic compound into a sterile carrier which contains a basic dispersion medium and the required other ingredients from those enumerated above. In the case of sterile powders for the preparation of sterile injectable solutions, the methods of preparation include vacuum drying and freeze-drying which yields a powder of the active ingredient (i.e., the therapeutic compound) plus any additional desired ingredient from a previously sterile-filtered solution thereof.

The therapeutic compound can be orally administered, for example, with an inert diluent or an assimilable edible carrier. The therapeutic compound and other ingredients may also be enclosed in a hard or soft shell gelatin capsule, compressed into tablets, or incorporated directly into the subject's diet. For oral therapeutic administration, the therapeutic compound may be incorporated with excipients and used in the form of ingestible tablets, buccal tablets, troches, capsules, elixirs, suspensions, syrups, wafers, and the like. The percentage of the therapeutic compound in the compositions and preparations may, of course, be varied. The amount of the therapeutic compound in such therapeutically useful compositions is such that a suitable dosage will be obtained.

It is especially advantageous to formulate parenteral compositions in dosage unit form for ease of administration and uniformity of dosage. Dosage unit form as used herein refers to physically discrete units suited as unitary dosages for the subjects to be treated; each unit containing a predetermined quantity of therapeutic compound calculated to produce the desired therapeutic effect in association with the required pharmaceutical carrier. The specification for the dosage unit forms of the disclosure are dictated by and directly dependent on (a) the unique characteristics of the therapeutic compound and the particular therapeutic effect to be achieved, and (b) the limitations inherent in the art of compounding such a therapeutic compound for the treatment of a selected condition in a patient.

The therapeutic compound may also be administered topically to the skin, eye, or mucosa. Alternatively, if local delivery to the lungs is desired the therapeutic compound may be administered by inhalation in a dry-powder or aerosol formulation. Alternatively, the therapeutic agents may be administered transdermally.

Active compounds are administered at a therapeutically effective dosage sufficient to treat a condition associated with a condition in a patient. For example, the efficacy of a compound can be evaluated in an animal model system that may be predictive of efficacy in treating the disease in a human or another animal, such as the model systems shown in the examples and drawings.

An effective dose range of a therapeutic can be extrapolated from effective doses determined in animal studies for a variety of different animals. In general a human equivalent dose (HED) in mg/kg can be calculated in accordance with the following formula (see, e.g., Reagan-Shaw et al., FASEB J., 22(3):659-661, 2008, which is incorporated herein by reference):

HED (mg/kg)=Animal dose (mg/kg)×(Animal Kₘ/Human Kₘ)

Use of the Kₘ factors in conversion results in more accurate HED values, which are based on body surface area (BSA) rather than only on body mass. Kₘ values for humans and various animals are well known. For example, the Kₘ for an average 60 kg human (with a BSA of 1.6 m²) is 37, whereas a 20 kg child (BSA 0.8 m²) would have a Kₘ of 25. Kₘ for some relevant animal models are also well known, including: mice Kₘ of 3 (given a weight of 0.02 kg and BSA of 0.007); hamster Kₘ of 5 (given a weight of 0.08 kg and BSA of 0.02); rat Kₘ of 6 (given a weight of 0.15 kg and BSA of 0.025) and monkey Kₘ of 12 (given a weight of 3 kg and BSA of 0.24).

Precise amounts of the therapeutic composition depend on the judgment of the practitioner and are peculiar to each individual. Nonetheless, a calculated HED dose provides a general guide. Other factors affecting the dose include the physical and clinical state of the patient, the route of administration, the intended goal of treatment and the potency, stability and toxicity of the particular therapeutic formulation.

The actual dosage amount of a compound of the present disclosure or composition comprising a compound of the present disclosure administered to a subject may be determined by physical and physiological factors such as type of animal treated, age, sex, body weight, severity of condition, the type of disease being treated, previous or concurrent therapeutic interventions, idiopathy of the subject and on the route of administration. These factors may be determined by a skilled artisan. The practitioner responsible for administration will typically determine the concentration of active ingredient(s) in a composition and appropriate dose(s) for the individual subject. The dosage may be adjusted by the individual physician in the event of any complication.

An effective amount typically will vary from about 0.0001 mg/kg to about 1000 mg/kg, from about 0.001 mg/kg to about 100 mg/kg, from about 0.01 mg/kg to about 750 mg/kg, from about 0.001 mg/kg to about 50 mg/kg, from about 100 mg/kg to about 500 mg/kg, from about 1.0 mg/kg to about 250 mg/kg, from about 10.0 mg/kg to about 150 mg/kg, or from about 1.0 mg/kg to about 15 mg/kg in one or more dose administrations daily, for one or several days (depending of course of the mode of administration and the factors discussed above). Other suitable dose ranges include 1 mg to 10000 mg per day, 100 mg to 10000 mg per day, 500 mg to 10000 mg per day, and 500 mg to 1000 mg per day. In some particular embodiments, the amount is less than 10,000 mg per day with a range of 750 mg to 9000 mg per day.

In some embodiments, the agent which interacts with a chemokine receptor may be administered in an amount from about 1 mg/kg to about 10 mg/kg, or about 1 mg/kg to about 5 mg/kg, or about 2.5 mg/kg. The agent which interacts with a chemokine receptor may be Groβ or a derivative thereof. Similarly, the VLA-4 inhibitor may be administered in an amount from about 1 mg/kg to about 100 mg/kg, or about 1 mg/kg to about 50 mg/kg, or about 1 mg/kg to about 25 mg/kg, or about 1 mg/kg to about 15 mg/kg, or about 1 mg/kg to about 10 mg/kg, or about 1 mg/kg to about 5 mg/kg, or about 3 mg/kg. In some embodiments, a specific VLA-4 inhibitor such as a compound of formula I or a specific compound described in the examples such as firategrast or compound numbers 822, 823, 824, 825, 842, 1173, 1174, 1219, 1220, 1221, 1222, 1223, 1224, 1512, 1554, 1555, 1570, 1571, 1608, 1609, 1610, 1611, 1632, 1633, 1745, 1746, 1747, 2087, 009 or 2088 may be administered in a range of about 1 mg/kg to about 200 mg/kg, or about 50 mg/kg to about 200 mg/kg, or about 50 mg/kg to about 100 mg/kg, or about 75 mg/kg to about 100 mg/kg, or about 100 mg/kg.

The effective amount may be less than 1 mg/kg/day, less than 500 mg/kg/day, less than 250 mg/kg/day, less than 100 mg/kg/day, less than 50 mg/kg/day, less than 25 mg/kg/day or less than 10 mg/kg/day. It may alternatively be in the range of 1 mg/kg/day to 200 mg/kg/day.

In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

In certain embodiments, a pharmaceutical composition of the present disclosure may comprise, for example, at least about 0.1% of a compound of the present disclosure. In other embodiments, the compound of the present disclosure may comprise between about 1% to about 75% of the weight of the unit, or between about 25% to about 60%, for example, and any range derivable therein.

Single or multiple doses of the agents are contemplated. Desired time intervals for delivery of multiple doses can be determined by one of ordinary skill in the art employing no more than routine experimentation. As an example, subjects may be administered two doses daily at approximately 12 hour intervals. In some embodiments, the agent is administered once a day.

The agent(s) may be administered on a routine schedule. As used herein a routine schedule refers to a predetermined designated period of time. The routine schedule may encompass periods of time which are identical or which differ in length, as long as the schedule is predetermined. For instance, the routine schedule may involve administration twice a day, every day, every two days, every three days, every four days, every five days, every six days, a weekly basis, a monthly basis or any set number of days or weeks there-between. Alternatively, the predetermined routine schedule may involve administration on a twice daily basis for the first week, followed by a daily basis for several months, etc. In other embodiments, the disclosure provides that the agent(s) may be taken orally and that the timing of which is or is not dependent upon food intake. Thus, for example, the agent can be taken every morning and/or every evening, regardless of when the subject has eaten or will eat.

(v) Combinational Therapies

The present disclosure may relate to one or more agents used in combination with a VLA-4 antagonist. The present disclosure describes combinations of VLA-4 antagonists with other therapeutic modalities as combination therapies to increase the mobilization of hematopoietic stem cells.

To increase the mobilization of hematopoietic stem cells using the methods and compositions of the present disclosure, one would generally administer to the subject with a VLA-4 antagonist and at least one other therapy. These therapies would be provided in a combined amount effective to achieve an increased activity. This process may involve contacting the cells/subjects with the both agents/therapies at the same time, e.g., using a single composition or pharmacological formulation that includes both agents, or by contacting the cell/subject with two distinct compositions or formulations, at the same time, wherein one composition includes the VLA-4 antagonist and the other includes the other agent.

Alternatively, the individual compounds in the compositions described herein may precede or follow the other compound treatment by time intervals ranging from seconds to days. One would generally ensure that a significant period of time did not expire between the time of each delivery, such that the therapies would still be able to exert an advantageously combined effect on the cell/subject. In such instances, it is contemplated that one would administer both modalities within about 12-24 hours of each other, within about 6-12 hours of each other, with a delay time of only about 1-2 hours, or less than 1 hour. Additionally, the agent which interacts with a chemokine receptor may be administered about 10-15 minutes, about 5-10 minutes, or about 0-5 minutes prior to administration of the VLA-4 inhibitor. For example, the agent which interacts with a chemokine receptor may be administered from about 15 minutes, about 14 minutes, about 13 minutes, about 12 minutes, about 11 minutes, about 10 minutes, about 9 minutes, about 8 minutes, about 7 minutes, about 6 minutes, about 5 minutes, about 4 minutes, about 3 minutes, about 2 minutes, to about 1 minute, or any range derivable therein before the VLA-4 inhibitor. Alternatively, the components may be administered at the same time.

The compositions and combination of agents used in the methods described herein may be administered as a single bolus dose, a dose over time such as an infusion, as in intravenous, subcutaneous, or transdermal administration, or in multiple dosages. If infusion is used, the combination may be infused for about 15 minutes to about 6 hours. In one embodiment, the infusion may occur for the duration of length of the apheresis. Additionally, the compositions or combination may be administered once daily for multiple days including from 1 to 4 days.

Furthermore, the compositions or combinations may be administered to the patient for one day or less than one day and then HSPCs isolated from the patient. The compositions or combinations described herein may be administered and then HSPCs may be isolated about 1 hour, about 2 hours, about 3 hours, about 4 hours, about 5 hours, about 6 hours, about 7 hours, or about 8 hours following administration.

It also is conceivable that more than one administration of either the compound or the other therapy will be desired. Various combinations may be employed, where a compound of the present disclosure is “A,” and the other compound or therapy is “B,” as exemplified below:

A/B/A B/A/B B/B/A A/A/B B/A/A A/B/B B/B/B/A B/B/A/B
A/A/B/B A/B/A/B A/B/B/A B/B/A/A B/A/B/A B/A/A/B B/B/B/A
A/A/A/B B/A/A/A A/B/A/A A/A/B/A A/B/B/B B/A/B/B B/B/A/B

Other combinations are also contemplated. In some aspects of the present disclosure, the agent may be a CXC chemokine or a derivative thereof. Some non-limiting examples of the agent include Groβ, truncated Groβ (Groβt), plerixafor (AMD3100), a granulocyte-colony stimulating factor (G-CSF) such as filgrastim, PEG-filgrastim, or lenograstim, or an inhibitor of integrin α9β1 such as BOP (N-benzenesulfonyl)-L-prolyl-L-O-(1-pyrrolidinylcarbonyl)tyrosine.

In other embodiments, the compositions or methods used herein may be administered with an anti-cancer therapy such as those described below. The methods or compositions described herein may be used in conjunction with standard methods or variations as practiced by a person of ordinary skill in the art. These anti-cancer agents may be administered prior to and/or concomitant with the compositions or methods described herein. Some non-limiting examples of anti-cancer therapies which may be used herein include carmustine, etoposide, cytarabine, melphalan, cyclophosphamide, busulfan, thiotepa, bleomycin, platinum (cisplatin), cytarabine, cyclophosphamide, buside, daunorubicin, doxorubicin, agent ara-C, cyclosporin; Rituxan®; thalidomide; clofarabine; Velcade®; Antegren®; Ontak®; Revlimid® (thalidomide analog); Prochymal®; Genasense® (oblimersen sodium); Gleevec®; Glivec® (imatinib); tamibarotene; nelarabine; gallium nitrate; PT-100; Bexxar®; Zevalin®; pixantrone; Onco-TCS; and agents that are topoisomerase inhibitors, or another specific anti-cancer therapy.

(v)(a) Chemotherapy

The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis. Most chemotherapeutic agents fall into the following categories: alkylating agents, antimetabolites, antitumor antibiotics, mitotic inhibitors, and nitrosoureas.

Examples of chemotherapeutic agents include alkylating agents such as thiotepa and cyclophosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin γ₁ and calicheamicin ω₁; dynemicin, including dynemicin A; uncialamycin and derivatives thereof; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, or zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as folinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel and docetaxel; chlorambucil; gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; vinorelbine; mitoxantrone; teniposide; edatrexate; daunomycin; aminopterin; capecitabine; ibandronate; irinotecan (e.g., CPT-11); topoisomerase inhibitor RFS 2000; difluorometlhylornithine (DMFO); retinoids such as retinoic acid; capecitabine; cisplatin (CDDP), carboplatin, procarbazine, mechlorethamine, cyclophosphamide, camptothecin, ifosfamide, melphalan, chlorambucil, busulfan, nitrosourea, dactinomycin, daunorubicin, doxorubicin, bleomycin, plicomycin, mitomycin, etoposide (VP16), tamoxifen, raloxifene, estrogen receptor binding agents, paclitaxel, docetaxel, gemcitabine, vinorelbine, farnesyl-protein transferase inhibitors, transplatinum, 5-fluorouracil, vincristine, vinblastine, and methotrexate and pharmaceutically acceptable salts, acids or derivatives of any of the above.

(v)(b) Radiotherapy

Radiotherapy, also called radiation therapy, is the treatment of cancer and other diseases with ionizing radiation. Ionizing radiation deposits energy that injures or destroys cells in the area being treated by damaging their genetic material, making it impossible for these cells to continue to grow. Although radiation damages both cancer cells and normal cells, the latter are able to repair themselves and function properly.

Radiation therapy used according to the present disclosure may include, but is not limited to, the use of γ-rays, X-rays, and/or the directed delivery of radioisotopes to tumor cells. Other forms of DNA damaging factors are also contemplated such as microwaves and UV-irradiation. It is most likely that all of these factors induce a broad range of damage on DNA, on the precursors of DNA, on the replication and repair of DNA, and on the assembly and maintenance of chromosomes. Dosage ranges for X-rays range from daily doses of 12.9 to 51.6 mC/kg for prolonged periods of time (3 to 4 wk), to single doses of 0.516 to 1.55 C/kg. Dosage ranges for radioisotopes vary widely, and depend on the half-life of the isotope, the strength and type of radiation emitted, and the uptake by the neoplastic cells.

Radiotherapy may comprise the use of radiolabeled antibodies to deliver doses of radiation directly to the cancer site (radioimmunotherapy). Antibodies are highly specific proteins that are made by the body in response to the presence of antigens (substances recognized as foreign by the immune system). Some tumor cells contain specific antigens that trigger the production of tumor-specific antibodies. Large quantities of these antibodies can be made in the laboratory and attached to radioactive substances (a process known as radiolabeling). Once injected into the body, the antibodies actively seek out the cancer cells, which are destroyed by the cell-killing (cytotoxic) action of the radiation. This approach can minimize the risk of radiation damage to healthy cells.

Conformal radiotherapy uses the same radiotherapy machine, a linear accelerator, as the normal radiotherapy treatment but metal blocks are placed in the path of the x-ray beam to alter its shape to match that of the cancer. This ensures that a higher radiation dose is given to the tumor. Healthy surrounding cells and nearby structures receive a lower dose of radiation, so the possibility of side effects is reduced. A device called a multi-leaf collimator has been developed and may be used as an alternative to the metal blocks. The multi-leaf collimator consists of a number of metal sheets which are fixed to the linear accelerator. Each layer can be adjusted so that the radiotherapy beams can be shaped to the treatment area without the need for metal blocks. Precise positioning of the radiotherapy machine is very important for conformal radiotherapy treatment and a special scanning machine may be used to check the position of internal organs at the beginning of each treatment.

High-resolution intensity modulated radiotherapy also uses a multi-leaf collimator. During this treatment the layers of the multi-leaf collimator are moved while the treatment is being given. This method is likely to achieve even more precise shaping of the treatment beams and allows the dose of radiotherapy to be constant over the whole treatment area.

Although research studies have shown that conformal radiotherapy and intensity modulated radiotherapy may reduce the side effects of radiotherapy treatment, it is possible that shaping the treatment area so precisely could stop microscopic cancer cells just outside the treatment area being destroyed. This means that the risk of the cancer coming back in the future may be higher with these specialized radiotherapy techniques.

Scientists also are looking for ways to increase the effectiveness of radiation therapy. Two types of investigational drugs are being studied for their effect on cells undergoing radiation. Radiosensitizers make the tumor cells more likely to be damaged, and radioprotectors protect normal tissues from the effects of radiation. Hyperthermia, the use of heat, is also being studied for its effectiveness in sensitizing tissue to radiation.

(v)(c) Immunotherapy

In the context of cancer treatment, immunotherapeutics, generally, rely on the use of immune effector cells and molecules to target and destroy cancer cells. Trastuzumab (Herceptin™) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells. The combination of therapeutic modalities, i.e., direct cytotoxic activity and inhibition or reduction of ErbB2 would provide therapeutic benefit in the treatment of ErbB2 overexpressing cancers.

In one aspect of immunotherapy, the tumor cell must bear some marker that is amenable to targeting, i.e., is not present on the majority of other cells. Many tumor markers exist and any of these may be suitable for targeting in the context of the present disclosure. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155. An alternative aspect of immunotherapy is to combine anticancer effects with immune stimulatory effects. Immune stimulating molecules also exist including: cytokines such as IL-2, IL-4, IL-12, GM-CSF, γ-IFN, chemokines such as MIP-1, MCP-1, IL-8 and growth factors such as FLT3 ligand. Combining immune stimulating molecules, either as proteins or using gene delivery in combination with a tumor suppressor has been shown to enhance anti-tumor effects (Ju et al., 2000). Moreover, antibodies against any of these compounds may be used to target the anti-cancer agents discussed herein.

Examples of immunotherapies currently under investigation or in use are immune adjuvants e.g., Mycobacterium bovis, Plasmodium falciparum, dinitrochlorobenzene and aromatic compounds (U.S. Pat. Nos. 5,801,005 and 5,739,169; Hui and Hashimoto, 1998; Christodoulides, et al., 1998), cytokine therapy, e.g., interferons α, β, and γ; IL-1, GM-CSF and TNF (Bukowski, et al., 1998; Davidson, et al., 1998; Hellstrand, et al., 1998) gene therapy, e.g., TNF, IL-1, IL-2, p53 (Qin et al., 1998; Austin-Ward and Villaseca, 1998; U.S. Pat. Nos. 5,830,880 and 5,846,945) and monoclonal antibodies, e.g., anti-ganglioside GM2, anti-HER-2, anti-p185 (Pietras, et al., 1998; Hanibuchi, et al., 1998; U.S. Pat. No. 5,824,311). It is contemplated that one or more anti-cancer therapies may be employed with the gene silencing therapies described herein.

In active immunotherapy, an antigenic peptide, polypeptide or protein, or an autologous or allogenic tumor cell composition or “vaccine” is administered, generally with a distinct bacterial adjuvant (Ravindranath and Morton, 1991; Morton, et al., 1992; Mitchell, et al., 1990; Mitchell, et al., 1993).

In adoptive immunotherapy, the patient's circulating lymphocytes, or tumor infiltrated lymphocytes, are isolated in vitro, activated by lymphokines such as IL-2 or transduced with genes for tumor necrosis, and readministered (Rosenberg, et al., 1988; 1989).

### III. Compositions Comprising an Integrin Inhibitor and Agents which Interact with a Chemokine and Methods of Use Thereof

Disclosed herein are compositions and methods comprising a first agent which interacts with a chemokine, such as a CXCR2 agonist, a second agent which interacts with a chemokine, such as, a CXCR4 inhibitor, and a compound that act as integrin antagonists or inhibitor, such as α4β1 integrin (VLA-4) antagonists as well as compositions thereof. These compositions may result in the mobilization of progenitor and/or stem cells from bone marrow to peripheral circulation. Additionally, provided herein are methods for the treatment and/or prevention of disease using these two therapeutic agents in combination. These compositions described herein may be used to stimulate progenitor and/or stem cells and result in such stimulation in a shorter amount of time relative to either agent alone or other known agents or combinations. These compositions may also have the added advantage that they result in the mobilization in higher numbers, begin mobilization in a shorter period of time, over a more prolonged period of time, or mobilize increased numbers of early progenitor and/or stem cells, LSK-SLAM cells, CFU-C cells, or other progenitor and/or stem cells which are competent to achieve a successful engraftment into the patient. In some embodiments, these compositions may be used in improving the harvest of hematopoietic stem cells or progenitor cells. These methods, compositions, or uses are described in more detail below.

(i). Compounds and Synthetic Methods

In some embodiments, the compounds used in the compositions of the present disclosure include the compounds described in the Examples and claims listed below. All the synthesis methods described above can be further modified and optimized using the principles and techniques of organic chemistry as applied by a person skilled in the art. Such principles and techniques are taught, for example, in March's Advanced Organic Chemistry: Reactions, Mechanisms, and Structure (2007), which is incorporated by reference herein.

Compounds employed in methods of the disclosure may contain one or more asymmetrically-substituted carbon or nitrogen atoms, and may be isolated in optically active or racemic form. Thus, all chiral, diastereomeric, racemic form, epimeric form, and all geometric isomeric forms of a structure are intended, unless the specific stereochemistry or isomeric form is specifically indicated. Compounds may occur as racemates and racemic mixtures, single enantiomers, diastereomeric mixtures and individual diastereomers. In some embodiments, a single diastereomer is obtained. The chiral centers of the compounds of the present disclosure can have the S or the R configuration, as defined by the IUPAC 1974 Recommendations. For example, mixtures of stereoisomers may be separated using the techniques taught in the Examples section below, as well as modifications thereof. Tautomeric forms are also included as well as pharmaceutically acceptable salts of such isomers and tautomers.

Atoms making up the compounds of the present disclosure are intended to include all isotopic forms of such atoms. Compounds of the present disclosure include those with one or more atoms that have been isotopically modified or enriched, in particular those with pharmaceutically acceptable isotopes or those useful for pharmaceutically research. Isotopes, as used herein, include those atoms having the same atomic number but different mass numbers. By way of general example and without limitation, isotopes of hydrogen include deuterium and tritium, and isotopes of carbon include ¹³C and ¹⁴C. Similarly, it is contemplated that one or more carbon atom(s) of a compound of the present disclosure may be replaced by a silicon atom(s). Furthermore, it is contemplated that one or more oxygen atom(s) of a compound of the present disclosure may be replaced by a sulfur or selenium atom(s).

Compounds of the present disclosure may also exist in prodrug form. Since prodrugs are known to enhance numerous desirable qualities of pharmaceuticals (e.g., solubility, bioavailability, manufacturing, etc.), the compounds employed in some methods of the disclosure may, if desired, be delivered in prodrug form. Thus, the disclosure contemplates prodrugs of compounds of the present disclosure as well as methods of delivering prodrugs. Prodrugs of the compounds employed in the disclosure may be prepared by modifying functional groups present in the compound in such a way that the modifications are cleaved, either in routine manipulation or in vivo, to the parent compound. Accordingly, prodrugs include, for example, compounds described herein in which a hydroxy, amino, or carboxy group is bonded to any group that, when the prodrug is administered to a subject, cleaves to form a hydroxy, amino, or carboxylic acid, respectively. Additional details regarding pro-drugs may be found in Smith and Williams, 1988, the entire contents of which are hereby incorporated by reference. Smith and Williams Introduction to the Principles of Drug Design, Smith, H. J.; Wright, 2nd ed., London (1988).

It should be recognized that the particular anion or cation forming a part of any salt of this disclosure is not critical, so long as the salt, as a whole, is pharmacologically acceptable. Compounds useful in the disclosure which are amines, may be administered or prepared in the forms of their acid addition salts or metal complexes thereof. Suitable acid addition salts include salts of inorganic acids that are biocompatible, including HCl, HBr, sulfuric, phosphoric and the like, as well as organic acids such as acetic, propionic, butyric and the like, as well as acids containing more than one carboxyl group, such as oxalic, glutaric, adipic and the like. Compounds useful in the disclosure that are carboxylic acids or otherwise acidic may be administered or prepared in forms of salts formed from inorganic or organic bases that are physiologically compatible. Thus, these compounds may be prepared in the forms of their sodium, potassium, calcium, or magnesium salts as appropriate or may be salts with organic bases such as caffeine or ethylamine. These compounds also may be in the form of metal complexes. Additional examples of pharmaceutically acceptable salts and their methods of preparation and use are presented in Handbook of Pharmaceutical Salts: Properties, and Use (2002), which is incorporated herein by reference.

It should be further recognized that the compounds of the present disclosure include those that have been further modified to comprise substituents that are convertible to hydrogen in vivo. This includes those groups that may be convertible to a hydrogen atom by enzymological or chemical means including, but not limited to, hydrolysis and hydrogenolysis. Examples include hydrolyzable groups, such as acyl groups, groups having an oxycarbonyl group, amino acid residues, peptide residues, o-nitrophenylsulfenyl, trimethylsilyl, tetrahydropyranyl, diphenylphosphinyl, and the like. Examples of acyl groups include formyl, acetyl, trifluoroacetyl, and the like. Examples of groups having an oxycarbonyl group include ethoxycarbonyl, tert-butoxycarbonyl (—C(O)OC(CH₃)₃, Boc), benzyloxycarbonyl, p-methoxybenzyloxycarbonyl, vinyloxycarbonyl, β-(p-toluenesulfonyl)ethoxycarbonyl, and the like. Suitable amino acid residues include, but are not limited to, residues of Gly (glycine), Ala (alanine), Arg (arginine), Asn (asparagine), Asp (aspartic acid), Cys (cysteine), Glu (glutamic acid), His (histidine), lie (isoleucine), Leu (leucine), Lys (lysine), Met (methionine), Phe (phenylalanine), Pro (proline), Ser (serine), Thr (threonine), Trp (tryptophan), Tyr (tyrosine), Val (valine), Nva (norvaline), Hse (homoserine), 4-Hyp (4-hydroxyproline), 5-Hyl (5-hydroxylysine), Orn (ornithine) and R-Ala. Examples of suitable amino acid residues also include amino acid residues that are protected with a protecting group. Examples of suitable protecting groups include those typically employed in peptide synthesis, including acyl groups (such as formyl and acetyl), arylmethoxycarbonyl groups (such as benzyloxycarbonyl and p-nitrobenzyloxycarbonyl), tert-butoxycarbonyl groups (—C(O)OC(CH₃)₃, Boc), and the like. Suitable peptide residues include peptide residues comprising two to five amino acid residues. The residues of these amino acids or peptides can be present in stereochemical configurations of the D-form, the L-form or mixtures thereof. In addition, the amino acid or peptide residue may have an asymmetric carbon atom. Examples of suitable amino acid residues having an asymmetric carbon atom include residues of Ala, Leu, Phe, Trp, Nva, Val, Met, Ser, Lys, Thr and Tyr. Peptide residues having an asymmetric carbon atom include peptide residues having one or more constituent amino acid residues having an asymmetric carbon atom. Examples of suitable amino acid protecting groups include those typically employed in peptide synthesis, including acyl groups (such as formyl and acetyl), arylmethoxycarbonyl groups (such as benzyloxycarbonyl and p-nitrobenzyloxycarbonyl), tert-butoxycarbonyl groups (—C(O)OC(CH₃)₃), and the like. Other examples of substituents “convertible to hydrogen in vivo” include reductively eliminable hydrogenolyzable groups. Examples of suitable reductively eliminable hydrogenolyzable groups include, but are not limited to, arylsulfonyl groups (such as o-toluenesulfonyl); methyl groups substituted with phenyl or benzyloxy (such as benzyl, trityl and benzyloxymethyl); arylmethoxycarbonyl groups (such as benzyloxycarbonyl and o-methoxy-benzyloxycarbonyl); and haloethoxycarbonyl groups (such as β,β,β-trichloroethoxycarbonyl and β-iodoethoxycarbonyl).

Compounds of the disclosure may also have the advantage that they may be more efficacious than, be less toxic than, be longer acting than, be more potent than, produce fewer side effects than, be more easily absorbed than, and/or have a better pharmacokinetic profile (e.g., higher oral bioavailability and/or lower clearance) than, and/or have other useful pharmacological, physical, or chemical properties over, compounds known in the prior art, whether for use in the indications stated herein or otherwise.

In some aspects, the present disclosure relates to compositions containing one or more VLA-4 inhibitors and one or more agents which interacts with a chemokine such as a CXCR2 agonist, a CXCR4 inhibitor, or G-CSF. In some aspects, the present disclosure relates to compositions containing one or more VLA-4 inhibitors and at least two agents which interact with a chemokine. In some aspects, the present disclosure relates to compositions containing one or more VLA-4 inhibitors, at least one CXCR4 inhibitor and at least one CXCR2 agonist. These compositions may further comprise an excipient such as solvent or diluent which renders the composition suitable for administration via injection. In some embodiments, the components of these compositions may be formulated independently and then administered simultaneously to a patient. In other embodiments, these compositions are formulated with additional therapeutic agents or excipients. In other embodiments, these compositions consists substantially of, consists essentially of, or consists of one or more VLA-4 inhibitors, one or more agents which interact with a chemokine, and one or more excipients. Each of the compositions described herein contain a pharmaceutically effective amount of each of these agents combined. In particular, the compositions may contain a pharmaceutically effective combined amount of a VLA-4 inhibitor, a CXCR4 inhibitor and a CXCR2 agonist. The pharmaceutically effective combined amount results when each agent is present in an amount such that the effect of the combination results in increased activity relative to a similar amount of a single agent. In some embodiments, the effect of the combination results in an additive increase in activity. In some embodiments, the effect of the combination results in synergistic activity.

These compositions may be used in a variety of indications such as the mobilization of hematopoietic stem cells or progenitor cells. These indications include elevating the number of progenitor and/or stem cells which are circulating in the patient especially elevating the number of these cells in the peripheral blood of a patient. Alternatively, these compositions may be used to treat a patient with cancer including sensitizing the patient to a chemotherapy and/or radiotherapy, for the treatment of hematopoietic cancer such as leukemias, myelomas, or lymphoma, or for the harvesting of hematopoietic progenitor and/or stem cells which may be transplanted into a patient who has impaired production of hematopoietic progenitor and/or stem cells. A patient may have impaired production of hematopoietic progenitor and/or stem cells resulting from a high dose of chemotherapy, radiotherapy, another therapeutic agent, or a genetic abnormality. Alternatively, the compositions described herein may be used to mobilize pre-cancerous or cancerous cells from the bone marrow into the peripheral blood. In some embodiments, the mobilization of pre-cancerous or cancerous cells from the bone marrow is used to potentiate or increase the effects of a standard cancer therapy such as a chemotherapeutic and/or radiotherapy. Furthermore, each of these compositions may be used in the manufacture of medicament for these indications.

The compositions and methods described herein may include one or more additional agents that are therapeutically or nutritionally useful such as antibiotics, vitamins, herbal extracts, anti-inflammatories, glucose, antipyretics, analgesics, cyclophosphamide, recombinant stem cell factor (Stemgen®), granulocyte-macrophage colony stimulating factor (GM-CSF) (such as Leukine®, and Leucomax®), ETRX-101, TLK 199/TILENTRA™, Interleukin-1 (IL-1), Interleukin-3 (IL-3), Interleukin-8 (IL-8), PIXY-321 (GM-CSF/IL-3 fusion protein), macrophage inflammatory protein, thrombopoietin, or a similar agent. Additionally, the compositions may contain one or more agents prevents microbial growth to increase the storage of the composition. Such agents may be an anti-parasitic, an antifungal, an antibiotic, or anti-viral. Additionally, the compositions may further comprise one or more chemotherapeutic agents.

(i)(a) VLA-4 Inhibitors

The methods and compositions used herein may contain one or more VLA-4 inhibitors or VLA-4 antagonists. The terms VLA-4 inhibitor and VLA-4 antagonist are used interchangeably in the disclosure. Some non-limiting examples of VLA-4 inhibitors which may be used in the compositions and methods described herein include antibodies, such as humanized monoclonal antibody against α4, natalizumab (Antegren®) and small molecules such as those described in U.S. Pat. No. 5,510,332; WO 06/023396; WO 97/03094; WO 97/02289; WO 96/40781; WO 96/22966; WO 96/20216; WO 96/01644; WO 96/06108; WO 95/15973; WO 96/31206; WO 06/010054; WO 05/087760; WO 01/12186; WO 99/37605; WO 01/51487; WO 03/011288; WO 02/14272; WO 01/32610; and EP 0842943, the entire contents of which are hereby incorporated by reference. An example of a VLA-4 inhibitor that may be used herein is BIO5192 (also known as AMD15057) disclosed in PCT publication WO 01/12186, which is incorporated herein by reference. Alternatively, analogs of BIO5192, such as BIO1211, may be used. In other embodiments, the VLA-4 inhibitor is firategrast or a pharmaceutically acceptable salt thereof. Firategrast is a compound of the formula:

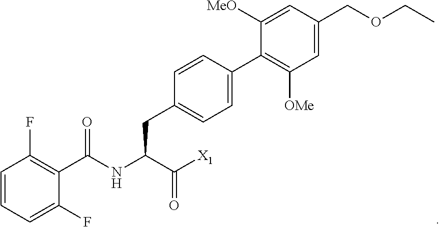

In another aspect, the compositions or methods described herein may contain one or more VLA-4 inhibitors with a structure shown below:

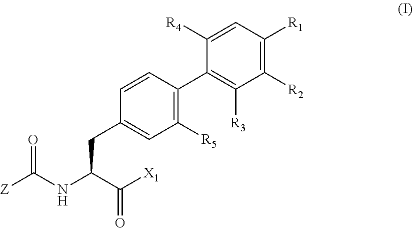

wherein:
   R₁ is hydrogen, aminocarbonyl, carboxy, cyano, halo, hydroxy, —SF₅, alkyl_{(C≤8)}, substituted alkyl_{(C≤8)}, alkoxy_{(C≤8)}, substituted alkoxy_{(C≤8)}, or —Y₁—Rₐ; wherein:
      Y₁ is alkanediyl_{(C≤8)} or substituted alkanediyl_{(C≤8)}; and
      Rₐ is alkoxy_{(C≤12)}, acyloxy_{(C≤12)}, or a substituted version of either of these groups; or
—X(CH₂O)ₘ—(CH₂CH₂O)ₙ—R_{b}; wherein:
   X is a covalent bond or —O—;
   m is 0 or 1;
   n is 1, 2, 3, 4, 5, 6, 7, or 8; and
   R_{b} is hydrogen, alkyl_{(C≤8)}, or substituted alkyl_{(C≤8)}; or
—CH₂—O—(CH₂)ₙ—R_{c}; wherein:
   N is 0, 1, or 2; and
   R_{c} is a cyclic aliphatic ring of 4-6 in size having one oxygen as part of the ring;
R₂ is hydrogen, aminocarbonyl, carboxy, cyano, halo, hydroxy, —SF₅, alkyl_{(C≤8)}, substituted alkyl_{(C≤8)}, alkoxy_{(C≤8)}, substituted alkoxy_{(C≤8)}, or —Y₂—R_{c}; wherein:
   Y₂ is alkanediyl_{(C≤8)} or substituted alkanediyl_{(C≤8)}; and
   R_{c} is alkoxy_{(C≤12)}, acyloxy_{(C≤12)}, or a substituted version of either of these groups;
X₁ is hydroxy, alkoxy_{(C≤8)}, substituted alkoxy_{(C≤8)}, cycloalkoxy_{(C≤8)}, substituted cycloalkoxy_{(C≤8)}, alkenyloxy_{(C≤8)}, substituted alkenyloxy_{(C≤8)}, aryloxy_{(C≤8)}, substituted aryloxy_{(C≤8)}, aralkoxy_{(C≤8)}, substituted aralkoxy_{(C≤8)}, or a substituent convertible in vivo to hydrogen; and
R₃ and R₄ are each independently hydrogen, hydroxy, alkoxy_{(C≤8)} or substituted alkoxy_{(C≤8)};
R₅ is hydrogen, —CH(OR_{d})Rₑ, or —C(O)R_{f}, wherein:
   R_{d} is hydrogen, alkyl_{(C≤8)}, or substituted alkyl_{(C≤8)}; and
   Rₑ and R_{f} are each independently alkyl_{(C≤8)} or substituted alkyl_{(C≤8)}; and
Z is a group of the formula:

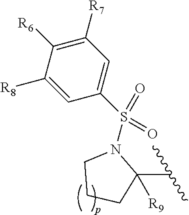

wherein:
   p is 0, 1, 2, or 3;
   R₆ is hydrogen or —C(O)X₂; wherein:
   X₂ is amino, hydroxy, alkoxy_{(C≤8)}, substituted alkoxy_{(C≤8)}, cycloalkoxy_{(C≤8)}, substituted cycloalkoxy_{(C≤8)}, alkenyloxy_{(C≤8)}, substituted alkenyloxy_{(C≤8)}, aryloxy_{(C≤8)}, substituted aryloxy_{(C≤8)}, aralkyloxy_{(C≤8)}, substituted aralkyloxy_{(C≤8)}, alkylamino_{(C≤8)}, substituted alkylamino_{(C≤8)}, dialkylamino_{(C≤8)}, substituted dialkylamino_{(C≤8)}, cycloalkyl-amino_{(C≤8)}, substituted cycloalkylamino_{(C≤8)}, alkenylamino_{(C≤8)}, substituted alkenylamino_{(C≤8)}, arylamino_{(C≤8)}, substituted arylamino_{(C≤8)}, aralkylamino_{(C≤8)}, substituted aralkylamino_{(C≤8)}, or a substituent convertible in vivo to hydrogen;
   R₇ and R₈ are each independently hydrogen, halo, haloalkyl_{(C≤8)}, or substituted haloalkyl_{(C≤8)};
   R₉ is hydrogen, alkyl_{(C≤8)}, or substituted alkyl_{(C≤8)}; or
a group of the formula:

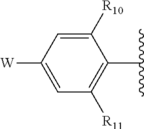

wherein:
   W is hydrogen, cyano, halo, hydroxy, or —C(O)X₃, wherein:
      X₃ is amino, hydroxy, alkoxy_{(C≤8)}, substituted alkoxy_{(C≤8)}, cycloalkoxy_{(C≤8)}, substituted cycloalkoxy_{(C≤8)}, alkenyloxy_{(C≤8)}, substituted alkenyloxy_{(C≤8)}, aryloxy_{(C≤8)}, substituted aryloxy_{(C≤8)}, aralkyloxy_{(C≤8)}, substituted aralkyloxy_{(C≤8)}, alkylamino_{(C≤8)}, substituted alkylamino_{(C≤8)}, dialkylamino_{(C≤8)}, substituted dialkylamino_{(C≤8)}, cycloalkylamino_{(C≤8)}, substituted cycloalkylamino_{(C≤8)}, alkenylamino_{(C≤8)}, substituted alkenyl-amino_{(C≤8)}, arylamino_{(C≤8)}, substituted arylamino_{(C≤8)}, aralkylamino_{(C≤8)}, substituted aralkylamino_{(C≤8)}, or a substituent convertible in vivo to hydrogen;
   R₁₀ and R₁₁ are each independently hydrogen or halo;
      or a pharmaceutically acceptable salt thereof.

(i)(b) an Agent which Interacts with a Chemokine

As used herein, the “agent which interacts with a chemokine” includes chemokines, cytokines, chemokine receptors, or an agent which modulates the activity of these molecules such as a fragment, an antibody, or a small organic molecule. As used herein “interacts with” means that the agent binds with a chemokine in a manner that modulates the activity of said chemokine, for example, by reducing, inhibiting, increasing or activating the activity of the chemokine. In one embodiment, the present disclosure relates to compositions which modulate the activity of a CXC chemokine receptor such as CXCR2 or CXCR4. In some embodiments, the present methods and compositions comprise at least two agents which interact with a chemokine. In some embodiments, the present methods and compositions comprise a first agent comprising a CXCR2 agonist and a second agent comprising a CXCR4 inhibitor. In a preferred embodiment, the methods and compositions comprise a VLA-4 inhibitor or VLA-4 antagonist, a CXCR4 inhibitor and a CXCR2 agonist. In a particular embodiment, the methods and compositions comprise a VLA-4 inhibitor or VLA-4 antagonist, AMD3100 and Groβ.

CXCR2 agonists include any molecule that activates the CXCR2 receptor. Such molecules include chemokines, cytokines, agonist antibodies or biologically active fragments thereof, or small organic molecules. Some non-limiting examples of chemokines acting via the CXCR2 receptor include, but are not limited to Groβ, Groα, Groγ, GCP-2 (granulocyte chemo-attractant protein 2), IL-8, NAP-2 (neutrophil activating peptide 2), ENA-78 (epithelial-cell derived neutrophil activating protein 78), and MGSA.

In some embodiments, the CXCR2 agonists used in the methods and compositions described herein are Groβ and modified forms thereof. King et al., Blood 2001; 97: 1534-1542 have demonstrated that a recombinant N-terminal 4-amino acid truncated form of the human chemokine Groβ (also known as SB-251353 or garnocestim or Groβt) can mobilize progenitor cells after administration of SB-251353 in combination with G-CSF. This combination resulted in the mobilization of neutrophils and platelets during these studies. Chemokines such as the SB-251353, Groα, Groβ, and Groγ are further discussed in WO 94/29341; WO 97/15594; WO 97/15595; WO 99/26645; WO 02/02132; U.S. Pat. Nos. 6,080,398; 6,399,053; and 6,447,766, which are incorporated herein by reference.

The “Groβ”, “Groβ protein”, or “Groβ chemokine” class includes Groβ itself as well as modified forms of Groβ. These modified forms include, but are not limited to, truncated, multimerized, amino-acid substituted, modified with amino-acid deletions and/or insertions, or combinations thereof. “Modified forms of Groβ” includes truncated forms such as those described in U.S. Pat. Nos. 6,447,766; 6,399,053; 6,080,398; PCT publication 99/26645; PCT publication WO 97/15595; PCT publication WO 02/02132; PCT publication WO 97/15594; and PCT publication WO 94/29341, which are incorporated herein by reference. “Modified forms of Groβ” are multimeric forms of Groβ such as dimers, trimers, tetramers, or other versions containing multiple proteins or modified proteins. Some non-limiting examples of “modified forms” include modified forms of Groβ with truncation of between 2 to about 8 amino acids at the amino terminus of the mature protein, truncation of between about 2 to about 10 amino acids at the carboxy terminus of the mature protein, or multimeric forms of the modified and/or truncated proteins, e.g., dimers, trimers, tetramers and other aggregated forms. Some non-limiting examples of truncated forms of Groβ may include SB-251353 which consists of amino acids 5-73 and forms thereof where amino acid 69 is deamidated.

Another specific CXCR2 receptor agonist that may be used in the compositions and methods described herein is SB-251353, a basic, heparin-binding protein with a molecular mass of approximately 7500 Da (King et al., J Immunol 2000; 164: 3774-3782, Hepburn et al., Journal of Pharmacology and Experimental Therapeutics 2001; 298: 886-893).

The compositions and methods described herein may comprise one or more CXCR4 inhibitors. Some non-limiting examples of CXCR4 inhibitors include AMD3100 (plerixafor), AMD3465, CTCE-0214, CTCE-9908, CP-1221 (linear peptides, cyclic peptides, natural amino-acids, unnatural amino acids, and peptidomimetic compounds), T140 and analogs, 4F-benzoyl-TN24003, KRH-1120, KRH-1636, KRH-2731, polyphemusin analogue, ALX40-4C, or a CXCR4 inhibitors described in WO 01/85196, WO 99/50461, WO 01/94420, WO 03/090512, US 2005/0059702, US 2005027767, US 2003/9229341, U.S. Pat. Nos. 5,021,409, 6,001,826, and 5,583,131, each of which is incorporated by reference herein.

The compositions or methods described herein may be comprise G-CSF. It is contemplated that any suitable source of G-CSF may be employed. The G-CSF used in the compositions or methods may be either recombinant or purified using known techniques and includes, but is not limited to, Neupogen® filgrastim (Amgen), Neutrogin®/Granocyte® lenograstim (Chugai Pharmaceuticals), and Neulasta® pegylated filgrastim (Amgen). Additionally, biologically active fragments, variants, derivatives or fusion proteins may also be employed provided these agents retain the ability to mobilize progenitor or stem cells.

(ii). Biological Activity

It is another object of the disclosure to provide pharmaceutical compositions comprising compounds described above. These compounds and pharmaceutical compositions may be used to improve the harvest of hematopoietic stem cells or progenitor cells. Additionally, the compounds or compositions may be used to elevate the circulation of hematopoietic progenitor and/or stem cells, improve the collection of hematopoietic stem cells or progenitor cells for a transfusion, increase the sensitization of an anti-cancer therapy such as a chemotherapeutic or radiotherapy, or mobilize pre-cancerous or cancerous cells into the peripheral blood which may increase their sensitivity to an anti-cancer therapy.

Hematopoietic stem cell transplant (HSCT) is used to facilitate repopulation of healthy bone marrow and immune system cells after high-dose chemotherapy treatment for cancers such as Hodgkin's and non-Hodgkin's lymphoma, multiple myeloma, and leukemia. In HSCT, hematopoietic stem/progenitor cells (HSPCs) are collected from the patient's blood, harvested, frozen and then stored while the patient receives high-dose chemotherapy and/or radiation therapy. Successful HSCT requires the intravenous infusion of a minimum number of 2×10⁶ CD34+ stem cells/kg body weight; however, a dose of 5×10⁶ CD34+ cells/kg is considered preferable for early and long term multilineage engraftment.

Stem cells harvested from peripheral blood are the most commonly used graft source in HSCT. While granulocyte colony-stimulating factor (G-CSF) is the most frequently used agent for stem cell mobilization, the use of G-CSF alone results in suboptimal stem cell yields in a significant proportion of patients. Plerixafor (AMD3100), a small molecule CXCR4 antagonist, in combination with G-CSF increases total CD34+ HSPCs compared to G-CSF alone and is FDA approved for stem cell mobilization in Non-Hodgkin's lymphoma and multiple myeloma. However, a significant disadvantage of plerixafor is cost, adding $25,567 per patient compared to G-CSF alone. Furthermore, up to 24% of patients receiving plerixafor and G-CSF still fail to collect ≥2×10⁶ CD34+ cells/kg in 4 days of apheresis. Recent economic analysis has determined that reducing apheresis by 1 day has the potential to decrease medical costs by $6,600. Thus improved/alternative mobilizing agents and strategies are needed.

Mechanistic studies have shown that the integrin α4β1 (VLA-4) plays an important role in the retention of HSPCs within the bone marrow (BM) microenvironment. HSPC mobilization has been achieved by disrupting the integrin α4β1N/CAM-1 axis with antibodies against integrin α4β1 or VCAM-1. Preclinical mouse studies in the DiPersio laboratory have shown that administration of the small molecule inhibitor of integrin α4β1, BIO5192, results in the rapid and reversible mobilization of HSPCs into the peripheral circulation with maximum mobilization occurring within 30 to 60 minutes and returning to baseline within 4 hours. A superior treatment could be envisioned wherein a patient receives an integrin α4β1 antagonist to continually inhibit integrin α4β1 over the course of ˜4 hours (average duration of CD34+ stem cell apheresis procedures), maximizing the mobilization of HSPCs that can be collected by apheresis during the same day of treatment.

BIO5192 is a potent small molecule inhibitor of integrin α4β1 and has demonstrated efficacy in mobilizing HSPC's in mice. However, BIO5192 has poor aqueous solubility, bioavailability, and pharmacokinetic properties and therefore has not been developed clinically. A simpler, more soluble integrin α4β1 antagonist is firategrast. Firategrast has been tested in clinical trials for the treatment of multiple sclerosis and has demonstrated efficacy in mobilizing HSPC's in mice but to a significantly lesser extent than BIO5192 and at much higher doses. Thus, development of integrin α4β1 antagonist which exhibit improved properties would greatly improve the clinical use of this particular combination.

Integrin antagonist compounds with high VLA-4 binding affinity and in vivo efficacy similar to BIO5192 but with superior pharmacokinetic and physiochemical properties are expected to increase mobilization of HSPCs alone or in a synergistic manner with G-CSF, plerixafor, truncated Gro-beta (Gro-βt) or other mobilization agents and thus remarkably improve the current HSCT regimen. These compositions or methods may also have the added advantage that the compositions or methods result in the mobilization in higher numbers, begin mobilization in a shorter period of time, over a more prolonged period of time, or mobilize increased numbers of early progenitor and/or stem cells, LSK-SLAM cells, CFU-C cells, or other progenitor and/or stem cells which are competent to achieve a successful engraftment into the patient. For example, the number of progenitor and/or stem cells mobilized when using the combination or methods described herein may be at least about 1.2-fold, at least about 1.5-fold, at least about 2-fold, at least about 2.5-fold, at least about 3-fold, at least about 3.5-fold, at least about 4-fold, at least about 4.5-fold, at least about 5-fold, at least about 5.5-fold, at least about 6-fold, at least about 6.5-fold, at least about 7-fold, at least about 7.5-fold, at least about 8-fold, at least about 8.5-fold, at least about 9-fold, at least about 9.5-fold, at least about 10-fold, at least about 11-fold, at least about 12-fold, at least about 13-fold, at least about 14-fold, or at least about 15-fold greater then when using a single agent alone. Specifically, the number of early progenitor and/or stem cells (e.g. LSK-SLAM cells) mobilized when using the combination of at least one VLA-4 inhibitor and at least one CXCR2 agonist is at least about 1.2-fold, at least about 1.5-fold, at least about 2-fold, at least about 2.5-fold, at least about 3-fold, at least about 3.5-fold, at least about 4-fold, at least about 4.5-fold, at least about 5-fold, at least about 5.5-fold, at least about 6-fold, at least about 6.5-fold, at least about 7-fold, at least about 7.5-fold, at least about 8-fold, at least about 8.5-fold, at least about 9-fold, at least about 9.5-fold, at least about 10-fold, at least about 11-fold, at least about 12-fold, at least about 13-fold, at least about 14-fold, at least about 15-fold, at least about 16-fold, at least about 17-fold, at least about 18-fold, at least about 19-fold, at least about 20-fold, at least about 21-fold, at least about 22-fold, at least about 23-fold, at least about 24-fold, or at least about 25-fold greater then when using a single agent alone.

(iii). Therapeutic Methods

In one aspect, this disclosure provides methods of inhibiting or antagonizing VLA-4 using one or more of the compounds disclosed herein, as well as pharmaceutical compositions thereof containing one or more VLA-4 antagonists in the presence of two or more agents that interact with a chemokine, such as Gro-β, AMD3100, or a derivatives thereof. The therapeutic methods described herein may be used to enhance or elevate the circulation of hematopoietic progenitor and/or stem cells. These therapeutic methods may be used to improve stem cell transplantation, tissue repair, improve the efficacy of cancer therapy, or other situations in which in vivo stimulation of hematopoiesis is desirable. The compositions or methods described herein wherein the VLA-4 inhibitor and agents which interact with a chemokine combine to act synergistically to induce rapid mobilization of progenitor and stem cells. For example, peak mobilization, when these combined therapeutic agents are used, may occur at about 10 minutes, about 15 minutes, about 20 minutes, about 25 minutes, about 30 minutes, about 45 minutes, about 1 hour, about 2 hours, about 3 hours, about 4 hours, about 5 hours, or about 6 hours after administration of the combination. In one embodiment, these compositions or methods result in a composition which acts synergistically to induce rapid mobilization of progenitor and stem cells with peak mobilization at about 15 minutes after administration of the combination. In contrast, this mobilization is significantly shorter than the 4-5 days needed to achieve maximum mobilization using G-CSF.

In one aspect, the methods and compositions described herein may be used to increase the harvest of HSPCs for a variety of different applications. These compositions and methods may be used to treat a patient who requires a transplantation. Alternatively, the compositions and methods may be used to treat a patient who does not require a transplantation. The patient who needs a transplant of HSPCs requires either an allogenic, autologous, or tandem transplant of HSPCs. In some embodiments, the HSPCs may be used in either allogenic or autologous transplants. In another aspect, the present methods and compositions described herein may be used to improve the circulation of cells to tissues which need repair. The increased circulation of HSPCs may be used to improve the repair of the target tissue in the patient.

If the HSPCs are harvested, these cells may be returned to the donor patient (autologous transplant) or may be donated to another patient that is sufficiently compatible to prevent rejection (allogeneic transplant). One non-limiting application of autologous transplantation is in combination with radiation or chemotherapy in patients bearing tumors since the radiotherapeutic or chemotherapeutic methods deplete the patient's normal cells. In this application, the patients cells may be harvested prior to or during the therapeutic treatments, fractionated if necessary, cultured and optionally expanded, and then returned to the patient to restore the damaged immune system depleted by the therapy. Allogeneic recipients may receive the cells for the same purpose, or may have a condition that may be benefited by enhancing their hematopoietic systems. In a typical protocol for these types of transplants, the mobilized cells are collected from the donor by, for example, apheresis and then stored/cultured/expanded/fractionated as desired. In some embodiments, the compositions or methods described herein may result in the need for aphersis being eliminated.

In some aspects, the present compositions and methods described herein may be used to increase the circulation of pre-cancerous or cancerous cells out of the bone marrow into the peripheral blood. Without wishing to be bound by any theory, it is believed that the increasing the circulation of pre-cancerous or cancerous cells out of the bone marrow may increase the effectiveness of an anti-cancer therapy. In particular, these methods or compositions may be used to treat patients who have or are at risk of a hematopoietic malignancy such as lymphoma, myeloma, or leukemia. The compositions or methods described herein may be administered or employed prior to, during, or subsequent to the anti-cancer therapy. Two non-limiting examples of anti-cancer therapies that may be used in the methods described herein or conjunction with the compositions described herein include chemotherapeutic agents or radiotherapy.

In another aspect, the compositions and methods described herein may be used to decrease inflammation which may result in increasing tissue repair. Thus, the compositions or methods described herein may be used to treat graft versus host disease. Additionally, these compositions or methods may be used to treat diseases or disorders associated with cell adhesion-mediated inflammatory pathways. Some non-limiting examples of cell adhesion-mediated inflammatory pathologies include asthma, multiple sclerosis, rheumatoid arthritis, atherosclerosis, and inflammatory bowel disease.

The pharmaceutical compositions of these combinations may further comprise one or more non-toxic, pharmaceutically acceptable carriers and/or diluents and/or adjuvants (collectively referred to herein as “carrier” materials) and if desired other active ingredients. These methods may be used to treat a blood disease or disorder such as sickle cell anemia or as a part of hematopoietic stem cell therapy to promote the development of stem cells. In some embodiments, the compound is administered as part of a pharmaceutical composition further comprising a pharmaceutically acceptable carrier. In some embodiments, the compounds and/or pharmaceutical compositions thereof may be administered orally, parenterally, or by inhalation spray, or topically in unit dosage formulations containing conventional pharmaceutically acceptable carriers, adjuvants and vehicles. The term parenteral as used herein includes, for example, subcutaneous, intravenous, intramuscular, intrasternal, infusion techniques or intraperitoneally. In some embodiments, the compounds of the present disclosure are administered by any suitable route in the form of a pharmaceutical composition adapted to such a route, and in a dose effective for the treatment intended. Therapeutically effective doses of the compounds required to prevent or arrest the progress of or to treat a medical condition are readily ascertained by one of ordinary skill in the art using preclinical and clinical approaches familiar to the medicinal arts.

Based upon standard laboratory experimental techniques and procedures well known and appreciated by those skilled in the art, as well as comparisons with compounds of known usefulness, the compounds described above can be used in the treatment of patients suffering from the above pathological conditions. One skilled in the art will recognize that selection of the most appropriate compound of the disclosure is within the ability of one with ordinary skill in the art and will depend on a variety of factors including assessment of results obtained in standard assay and animal models.

In several aspects of the present disclosure, the compounds provided herein may be used in a variety of biological, prophylactic or therapeutic areas, including those in wherein VLA-4 plays a role.

(iv). Pharmaceutical Formulations and Routes of Administration

For administration to an animal especially a mammal in need of such treatment, the compounds in a therapeutically effective amount are ordinarily combined with one or more excipients appropriate to the indicated route of administration. The compounds of the present disclosure are contemplated to be formulated in a manner ameniable to treatment of a veterinary patient as well as a human patient. In some embodiments, the veterinary patient may be an avian such as chicken, turkey, or duck, a companion animal such as a cat or dog, livestock animals such as a cow, horse, pig, or goat, zoo animals, and wild animals The compounds may be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia, sodium alginate, polyvinylpyrrolidone, and/or polyvinyl alcohol, and tableted or encapsulated for convenient administration. Alternatively, the compounds may be dissolved in water, polyethylene glycol, propylene glycol, ethanol, corn oil, cottonseed oil, peanut oil, sesame oil, benzyl alcohol, sodium chloride, and/or various buffers. Other excipients and modes of administration are well and widely known in the pharmaceutical art and may be adapted to the type of animal being treated. Description of potential administration routes which may be used to formulate the compositions described herein include those taught in Remington's Pharmaceutical Sciences, latest edition, Mack Publishing Company, Easton, Pa., which is incorporated herein by reference.

The pharmaceutical compositions useful in the present disclosure may be subjected to conventional pharmaceutical operations such as sterilization and/or may contain conventional pharmaceutical carriers and excipients such as preservatives, stabilizers, wetting agents, emulsifiers, buffers, etc.

The compounds of the present disclosure may be administered by a variety of methods, e.g., orally or by injection (e.g. subcutaneous, intravenous, intraperitoneal, etc.). Depending on the route of administration, the active compounds may be coated in a material to protect the compound from the action of acids and other natural conditions which may inactivate the compound. They may also be administered by continuous perfusion/infusion of a disease or wound site.

In one aspect, the present methods or compositions may be administered such that the VLA-4 inhibitor is administered intravenously and the first and/or second agent which interacts with a chemokine is administered subcutaneously. Alternatively, the VLA-4 inhibitor and the first and/or second agent which interacts with a chemokine may be administered subcutaneously. In still another embodiment, both the VLA-4 inhibitor and the first and/or second agent which interacts with a chemokine are administered subcutaneously in a single formulation. In some embodiments, the VLA-4 inhibitor and the first and/or second agent which interacts with a chemokine are administered as a single formulation subcutaneously or intravenously. The protocols for administration to a particular patient may be further optimized by a skilled practitioner.

To administer the therapeutic compound by other than parenteral administration, it may be necessary to coat the compound with, or co-administer the compound with, a material to prevent its inactivation. For example, the therapeutic compound may be administered to a patient in an appropriate carrier, for example, liposomes, or a diluent. Pharmaceutically acceptable diluents include saline and aqueous buffer solutions. Liposomes include water-in-oil-in-water CGF emulsions as well as conventional liposomes.

The therapeutic compound may also be administered parenterally, intraperitoneally, intramuscularly, intraarterially, intraspinally, or intracerebrally. Dispersions can be prepared in glycerol, liquid polyethylene glycols, and mixtures thereof and in oils. Under ordinary conditions of storage and use, these preparations may contain a preservative to prevent the growth of microorganisms.

Pharmaceutical compositions may be suitable for injectable use include sterile aqueous solutions (where water soluble) or dispersions and sterile powders for the extemporaneous preparation of sterile injectable solutions or dispersion. In all cases, the composition must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (such as, glycerol, propylene glycol, and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it may be useful to include isotonic agents, for example, sugars, sodium chloride, or polyalcohols such as mannitol and sorbitol, in the composition. Prolonged absorption of the injectable compositions can be brought about by including in the composition an agent which delays absorption, for example, aluminum monostearate or gelatin. For intravenous or parenteral administration, the compounds are formulated in suitable liquid form with excipients as required. The compositions may contain liposomes or other suitable carriers. For injection intravenously, the solution is made isotonic using standard preparations such as Hank's solution or other isotonic solutions.

Sterile injectable solutions can be prepared by incorporating the therapeutic compound in the required amount in an appropriate solvent with one or a combination of ingredients enumerated above, as required, followed by filtered sterilization. Generally, dispersions are prepared by incorporating the therapeutic compound into a sterile carrier which contains a basic dispersion medium and the required other ingredients from those enumerated above. In the case of sterile powders for the preparation of sterile injectable solutions, the methods of preparation include vacuum drying and freeze-drying which yields a powder of the active ingredient (i.e., the therapeutic compound) plus any additional desired ingredient from a previously sterile-filtered solution thereof.

The therapeutic compound can be orally administered, for example, with an inert diluent or an assimilable edible carrier. The therapeutic compound and other ingredients may also be enclosed in a hard or soft shell gelatin capsule, compressed into tablets, or incorporated directly into the subject's diet. For oral therapeutic administration, the therapeutic compound may be incorporated with excipients and used in the form of ingestible tablets, buccal tablets, troches, capsules, elixirs, suspensions, syrups, wafers, and the like. The percentage of the therapeutic compound in the compositions and preparations may, of course, be varied. The amount of the therapeutic compound in such therapeutically useful compositions is such that a suitable dosage will be obtained.

It is especially advantageous to formulate parenteral compositions in dosage unit form for ease of administration and uniformity of dosage. Dosage unit form as used herein refers to physically discrete units suited as unitary dosages for the subjects to be treated; each unit containing a predetermined quantity of therapeutic compound calculated to produce the desired therapeutic effect in association with the required pharmaceutical carrier. The specification for the dosage unit forms of the disclosure are dictated by and directly dependent on (a) the unique characteristics of the therapeutic compound and the particular therapeutic effect to be achieved, and (b) the limitations inherent in the art of compounding such a therapeutic compound for the treatment of a selected condition in a patient.

The therapeutic compound may also be administered topically to the skin, eye, or mucosa. Alternatively, if local delivery to the lungs is desired the therapeutic compound may be administered by inhalation in a dry-powder or aerosol formulation. Alternatively, the therapeutic agents may be administered transdermally.

Active compounds are administered at a therapeutically effective dosage sufficient to treat a condition associated with a condition in a patient. For example, the efficacy of a compound can be evaluated in an animal model system that may be predictive of efficacy in treating the disease in a human or another animal, such as the model systems shown in the examples and drawings.

An effective dose range of a therapeutic can be extrapolated from effective doses determined in animal studies for a variety of different animals. In general a human equivalent dose (HED) in mg/kg can be calculated in accordance with the following formula (see, e.g., Reagan-Shaw et al., FASEB J., 22(3):659-661, 2008, which is incorporated herein by reference):

HED (mg/kg)=Animal dose (mg/kg)×(Animal Kₘ/Human Kₘ)

Use of the Kₘ factors in conversion results in more accurate HED values, which are based on body surface area (BSA) rather than only on body mass. Kₘ values for humans and various animals are well known. For example, the Kₘ for an average 60 kg human (with a BSA of 1.6 m²) is 37, whereas a 20 kg child (BSA 0.8 m²) would have a Kₘ of 25. Kₘ for some relevant animal models are also well known, including: mice Kₘ of 3 (given a weight of 0.02 kg and BSA of 0.007); hamster Kₘ of 5 (given a weight of 0.08 kg and BSA of 0.02); rat Kₘ of 6 (given a weight of 0.15 kg and BSA of 0.025) and monkey Kₘ of 12 (given a weight of 3 kg and BSA of 0.24).

Precise amounts of the therapeutic composition depend on the judgment of the practitioner and are peculiar to each individual. Nonetheless, a calculated HED dose provides a general guide. Other factors affecting the dose include the physical and clinical state of the patient, the route of administration, the intended goal of treatment and the potency, stability and toxicity of the particular therapeutic formulation.

The actual dosage amount of a compound of the present disclosure or composition comprising a compound of the present disclosure administered to a subject may be determined by physical and physiological factors such as type of animal treated, age, sex, body weight, severity of condition, the type of disease being treated, previous or concurrent therapeutic interventions, idiopathy of the subject and on the route of administration. These factors may be determined by a skilled artisan. The practitioner responsible for administration will typically determine the concentration of active ingredient(s) in a composition and appropriate dose(s) for the individual subject. The dosage may be adjusted by the individual physician in the event of any complication.

An effective amount typically will vary from about 0.0001 mg/kg to about 1000 mg/kg, from about 0.001 mg/kg to about 100 mg/kg, from about 0.01 mg/kg to about 750 mg/kg, from about 0.001 mg/kg to about 50 mg/kg, from about 100 mg/kg to about 500 mg/kg, from about 1.0 mg/kg to about 250 mg/kg, from about 10.0 mg/kg to about 150 mg/kg, or from about 1.0 mg/kg to about 15 mg/kg in one or more dose administrations daily, for one or several days (depending of course of the mode of administration and the factors discussed above). Other suitable dose ranges include 1 mg to 10000 mg per day, 100 mg to 10000 mg per day, 500 mg to 10000 mg per day, and 500 mg to 1000 mg per day. In some particular embodiments, the amount is less than 10,000 mg per day with a range of 750 mg to 9000 mg per day.

In some embodiments, an agent which interacts with a chemokine may be administered in an amount from about 1 mg/kg to about 10 mg/kg, or about 1 mg/kg to about 5 mg/kg, or about 2.5 mg/kg. The agents which interact with a chemokine may be Groβ, a CXCR4 inhibitor or derivatives thereof. Similarly, the VLA-4 inhibitor may be administered in an amount from about 1 mg/kg to about 100 mg/kg, or about 1 mg/kg to about 50 mg/kg, or about 1 mg/kg to about 25 mg/kg, or about 1 mg/kg to about 15 mg/kg, or about 1 mg/kg to about 10 mg/kg, or about 1 mg/kg to about 5 mg/kg, or about 3 mg/kg. In some embodiments, a specific VLA-4 inhibitor such as a compound of formula I or a specific compound described in the examples such as firategrast or compound numbers 12, 21, 22, 23, 24, 25, 26, 33, 34, 35, 36, 42, 43, 44, 45, or 49 may be administered in a range of about 1 mg/kg to about 200 mg/kg, or about 50 mg/kg to about 200 mg/kg, or about 50 mg/kg to about 100 mg/kg, or about 75 mg/kg to about 100 mg/kg, or about 100 mg/kg.

The effective amount may be less than 1 mg/kg/day, less than 500 mg/kg/day, less than 250 mg/kg/day, less than 100 mg/kg/day, less than 50 mg/kg/day, less than 25 mg/kg/day or less than 10 mg/kg/day. It may alternatively be in the range of 1 mg/kg/day to 200 mg/kg/day.

In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

In certain embodiments, a pharmaceutical composition of the present disclosure may comprise, for example, at least about 0.1% of a compound of the present disclosure. In other embodiments, the compound of the present disclosure may comprise between about 1% to about 75% of the weight of the unit, or between about 25% to about 60%, for example, and any range derivable therein.

Single or multiple doses of the agents are contemplated. Desired time intervals for delivery of multiple doses can be determined by one of ordinary skill in the art employing no more than routine experimentation. As an example, subjects may be administered two doses daily at approximately 12 hour intervals. In some embodiments, the agent is administered once a day.

The agent(s) may be administered on a routine schedule. As used herein a routine schedule refers to a predetermined designated period of time. The routine schedule may encompass periods of time which are identical or which differ in length, as long as the schedule is predetermined. For instance, the routine schedule may involve administration twice a day, every day, every two days, every three days, every four days, every five days, every six days, a weekly basis, a monthly basis or any set number of days or weeks there-between. Alternatively, the predetermined routine schedule may involve administration on a twice daily basis for the first week, followed by a daily basis for several months, etc. In other embodiments, the disclosure provides that the agent(s) may taken orally and that the timing of which is or is not dependent upon food intake. Thus, for example, the agent can be taken every morning and/or every evening, regardless of when the subject has eaten or will eat.

(v). Combination Therapies

The present disclosure relates to two or more agents used in combination with a VLA-4 antagonist. It is common in the field of mobilizing hematopoietic stem cells to combine therapeutic modalities. The present disclosure describes combinations of VLA-4 antagonists with other therapeutic modalities as combination therapies to increase the mobilization of hematopoietic stem cells.

To increase the mobilization of hematopoietic stem cells using the methods and compositions of the present disclosure, one would generally administer to the subject with a VLA-4 antagonist and at least two other therapies. These therapies would be provided in a combined amount effective to achieve an increased activity. This process may involve contacting the cells/subjects with the both agents/therapies at the same time, e.g., using a single composition or pharmacological formulation that includes the agents, or by contacting the cell/subject with two or more distinct compositions or formulations, at the same time, wherein one composition includes the VLA-4 antagonist, one composition includes a first agent and another composition that includes a second agent.

Alternatively, the individual compounds in the compositions described herein may precede or follow the other compound treatment by time intervals ranging from seconds to days. One would generally ensure that a significant period of time did not expire between the time of each delivery, such that the therapies would still be able to exert an advantageously combined effect on the cell/subject. In such instances, it is contemplated that one would administer the modalities within about 12-24 hours of each other, within about 6-12 hours of each other, with a delay time of only about 1-2 hours, or less than 1 hour. Additionally, the agents which interact with a chemokine may be administered about 10-15 minutes, about 5-10 minutes, or about 0-5 minutes prior to administration of the VLA-4 inhibitor. For example, the agents which interact with a chemokine may be administered from about 15 minutes, about 14 minutes, about 13 minutes, about 12 minutes, about 11 minutes, about 10 minutes, about 9 minutes, about 8 minutes, about 7 minutes, about 6 minutes, about 5 minutes, about 4 minutes, about 3 minutes, about 2 minutes, to about 1 minute, or any range derivable therein before the VLA-4 inhibitor. Alternatively, the components may be administered at the same time.

The compositions and combination of agents used in the methods described herein may be administered as a single bolus dose, a dose over time such as an infusion, as in intravenous, subcutaneous, or transdermal administration, or in multiple dosages. If infusion is used, the combination may be infused for about 15 minutes to about 6 hours. In one embodiment, the infusion may occur for the duration of length of the apheresis. Additionally, the compositions or combination may be administered once daily for multiple days including from 1 to 4 days.

Furthermore, the compositions or combinations may be administered to the patient for one day or less than one day and then HSPCs isolated from the patient. The compositions or combinations described herein may be administered and then HSPCs may be isolated about 1 hour, about 2 hours, about 3 hours, about 4 hours, about 5 hours, about 6 hours, about 7 hours, or about 8 hours following administration.

It also is conceivable that more than one administration of either the compound or the other therapy will be desired. Various combinations may be employed, where a compound of the present disclosure is “A,” and the other compound or therapy is “B,” as exemplified below:

A/B/A B/A/B B/B/A A/A/B B/A/A A/B/B B/B/B/A B/B/A/B
A/A/B/B A/B/A/B A/B/B/A B/B/A/A B/A/B/A B/A/A/B B/B/B/A
A/A/A/B B/A/A/A A/B/A/A A/A/B/A A/B/B/B B/A/B/B B/B/A/B

Other combinations are also contemplated. In some aspects of the present disclosure, the agent may be a CXC chemokine, a CXC chemokine receptor, or a derivative thereof. Some non-limiting examples of agent including Gro-β, truncated Gro-β (tGro-β), plerixafor (AMD3100), a granulocyte-colony stimulating factor (G-CSF) such as filgrastim, PEG-filgrastim, or lenograstim, or an inhibitor of integrin α9β1 such as BOP (N-benzenesulfonyl)-L-prolyl-L-O-(1-pyrrolidinylcarbonyl)tyrosine.

In other embodiments, the compositions or methods used herein may be administered with an anti-cancer therapy such as those described below. The methods or compositions described herein may be used in conjunction with standard methods or variations as practiced by a person of ordinary skill in the art. These anti-cancer agents may be administered prior to and/or concomitant with the compositions or methods described herein. Some non-limiting examples of anti-cancer therapies which may be used herein include carmustine, etoposide, cytarabine, melphalan, cyclophosphamide, busulfan, thiotepa, bleomycin, platinum (cisplatin), cytarabine, cyclophosphamide, buside, cytoxan, daunorubicin, doxorubicin, agent ara-C, cyclosporin; Rituxan®; thalidomide; clofarabine; Velcade®; Antegren®; Ontak®; Revlimid® (thalidomide analog); Prochymal™; Genasense® (oblimersen sodium); Gleevec™; Glivec® (imatinib); tamibarotene; nelarabine; gallium nitrate; PT-100; Bexxar®; Zevalin®; pixantrone; Onco-TCS; and agents that are topoisomerase inhibitors, or another specific anti-cancer therapy.

(v)(i) Chemotherapy

The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis. Most chemotherapeutic agents fall into the following categories: alkylating agents, antimetabolites, antitumor antibiotics, mitotic inhibitors, and nitrosoureas.

Examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin γ₁ and calicheamicin ω₁; dynemicin, including dynemicin A; uncialamycin and derivatives thereof; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-l-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, or zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as folinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel and docetaxel; chlorambucil; gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DMFO); retinoids such as retinoic acid; capecitabine; cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, paclitaxel, docetaxel, gemcitabien, navelbine, farnesyl-protein tansferase inhibitors, transplatinum, 5-fluorouracil, vincristin, vinblastin and methotrexate and pharmaceutically acceptable salts, acids or derivatives of any of the above.

(v)(ii) Radiotherapy

Radiotherapy, also called radiation therapy, is the treatment of cancer and other diseases with ionizing radiation. Ionizing radiation deposits energy that injures or destroys cells in the area being treated by damaging their genetic material, making it impossible for these cells to continue to grow. Although radiation damages both cancer cells and normal cells, the latter are able to repair themselves and function properly.

Radiation therapy used according to the present disclosure may include, but is not limited to, the use of γ-rays, X-rays, and/or the directed delivery of radioisotopes to tumor cells. Other forms of DNA damaging factors are also contemplated such as microwaves and UV-irradiation. It is most likely that all of these factors induce a broad range of damage on DNA, on the precursors of DNA, on the replication and repair of DNA, and on the assembly and maintenance of chromosomes. Dosage ranges for X-rays range from daily doses of 12.9 to 51.6 mC/kg for prolonged periods of time (3 to 4 wk), to single doses of 0.516 to 1.55 C/kg. Dosage ranges for radioisotopes vary widely, and depend on the half-life of the isotope, the strength and type of radiation emitted, and the uptake by the neoplastic cells.

Radiotherapy may comprise the use of radiolabeled antibodies to deliver doses of radiation directly to the cancer site (radioimmunotherapy). Antibodies are highly specific proteins that are made by the body in response to the presence of antigens (substances recognized as foreign by the immune system). Some tumor cells contain specific antigens that trigger the production of tumor-specific antibodies. Large quantities of these antibodies can be made in the laboratory and attached to radioactive substances (a process known as radiolabeling). Once injected into the body, the antibodies actively seek out the cancer cells, which are destroyed by the cell-killing (cytotoxic) action of the radiation. This approach can minimize the risk of radiation damage to healthy cells.

Conformal radiotherapy uses the same radiotherapy machine, a linear accelerator, as the normal radiotherapy treatment but metal blocks are placed in the path of the x-ray beam to alter its shape to match that of the cancer. This ensures that a higher radiation dose is given to the tumor. Healthy surrounding cells and nearby structures receive a lower dose of radiation, so the possibility of side effects is reduced. A device called a multi-leaf collimator has been developed and may be used as an alternative to the metal blocks. The multi-leaf collimator consists of a number of metal sheets which are fixed to the linear accelerator. Each layer can be adjusted so that the radiotherapy beams can be shaped to the treatment area without the need for metal blocks. Precise positioning of the radiotherapy machine is very important for conformal radiotherapy treatment and a special scanning machine may be used to check the position of internal organs at the beginning of each treatment.

High-resolution intensity modulated radiotherapy also uses a multi-leaf collimator. During this treatment the layers of the multi-leaf collimator are moved while the treatment is being given. This method is likely to achieve even more precise shaping of the treatment beams and allows the dose of radiotherapy to be constant over the whole treatment area.

Although research studies have shown that conformal radiotherapy and intensity modulated radiotherapy may reduce the side effects of radiotherapy treatment, it is possible that shaping the treatment area so precisely could stop microscopic cancer cells just outside the treatment area being destroyed. This means that the risk of the cancer coming back in the future may be higher with these specialized radiotherapy techniques.

Scientists also are looking for ways to increase the effectiveness of radiation therapy. Two types of investigational drugs are being studied for their effect on cells undergoing radiation. Radiosensitizers make the tumor cells more likely to be damaged, and radioprotectors protect normal tissues from the effects of radiation. Hyperthermia, the use of heat, is also being studied for its effectiveness in sensitizing tissue to radiation.

(v)(iii) Immunotherapy

In the context of cancer treatment, immunotherapeutics, generally, rely on the use of immune effector cells and molecules to target and destroy cancer cells. Trastuzumab (Herceptin™) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells. The combination of therapeutic modalities, i.e., direct cytotoxic activity and inhibition or reduction of ErbB2 would provide therapeutic benefit in the treatment of ErbB2 overexpressing cancers.

In one aspect of immunotherapy, the tumor cell must bear some marker that is amenable to targeting, i.e., is not present on the majority of other cells. Many tumor markers exist and any of these may be suitable for targeting in the context of the present disclosure. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155. An alternative aspect of immunotherapy is to combine anticancer effects with immune stimulatory effects. Immune stimulating molecules also exist including: cytokines such as IL-2, IL-4, IL-12, GM-CSF, γ-IFN, chemokines such as MIP-1, MCP-1, IL-8 and growth factors such as FLT3 ligand. Combining immune stimulating molecules, either as proteins or using gene delivery in combination with a tumor suppressor has been shown to enhance anti-tumor effects (Ju et al., 2000). Moreover, antibodies against any of these compounds may be used to target the anti-cancer agents discussed herein.

Examples of immunotherapies currently under investigation or in use are immune adjuvants e.g., Mycobacterium bovis, Plasmodium falciparum, dinitrochlorobenzene and aromatic compounds (U.S. Pat. Nos. 5,801,005 and 5,739,169; Hui and Hashimoto, 1998; Christodoulides, et al., 1998), cytokine therapy, e.g., interferons α, β, and γ; IL-1, GM-CSF and TNF (Bukowski, et al., 1998; Davidson, et al., 1998; Hellstrand, et al., 1998) gene therapy, e.g., TNF, IL-1, IL-2, p53 (Qin et al., 1998; Austin-Ward and Villaseca, 1998; U.S. Pat. Nos. 5,830,880 and 5,846,945) and monoclonal antibodies, e.g., anti-ganglioside GM2, anti-HER-2, anti-p185 (Pietras, et al., 1998; Hanibuchi, et al., 1998; U.S. Pat. No. 5,824,311). It is contemplated that one or more anti-cancer therapies may be employed with the gene silencing therapies described herein.

In active immunotherapy, an antigenic peptide, polypeptide or protein, or an autologous or allogenic tumor cell composition or “vaccine” is administered, generally with a distinct bacterial adjuvant (Ravindranath and Morton, 1991; Morton, et al., 1992; Mitchell, et al., 1990; Mitchell, et al., 1993).

In adoptive immunotherapy, the patient's circulating lymphocytes, or tumor infiltrated lymphocytes, are isolated in vitro, activated by lymphokines such as IL-2 or transduced with genes for tumor necrosis, and readministered (Rosenberg, et al., 1988; 1989).

### IV. Definitions

When used in the context of a chemical group: “hydrogen” means —H; “hydroxy” means —OH; “oxo” means ═O; “carbonyl” means —C(═O)—; “carboxy” means —C(═O)OH (also written as —COOH or —CO₂H); “halo” means independently —F, —Cl, —Br or —I; “amino” means —NH₂; “hydroxyamino” means —NHOH; “nitro” means —NO₂; imino means ═NH; “cyano” means —CN; “isocyanate” means —N═C═O; “azido” means —N₃; in a monovalent context “phosphate” means —OP(O)(OH)₂ or a deprotonated form thereof; in a divalent context “phosphate” means —OP(O)(OH)O— or a deprotonated form thereof; “mercapto” means —SH; and “thio” means ═S; “sulfonyl” means —S(O)₂—; and “sulfinyl” means —S(O)—.

In the context of chemical formulas, the symbol “—” means a single bond, “═” means a double bond, and “≡” means triple bond. The symbol “ [symbol] ” represents an optional bond, which if present is either single or double. The symbol “ [symbol] ” represents a single bond or a double bond. Thus, the formula

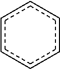

covers, for example,

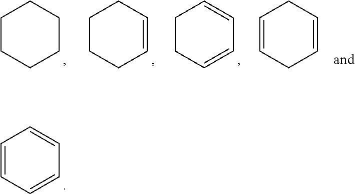

And it is understood that no one such ring atom forms part of more than one double bond. Furthermore, it is noted that the covalent bond symbol “—”, when connecting one or two stereogenic atoms, does not indicate any preferred stereochemistry. Instead, it covers all stereoisomers as well as mixtures thereof. The symbol “ [symbol] ”, when drawn perpendicularly across a bond (e.g.,

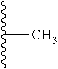

for methyl) indicates a point of attachment of the group. It is noted that the point of attachment is typically only identified in this manner for larger groups in order to assist the reader in unambiguously identifying a point of attachment. The symbol “ [symbol] ” means a single bond where the group attached to the thick end of the wedge is “out of the page.” The symbol “ [symbol] ” means a single bond where the group attached to the thick end of the wedge is “into the page”. The symbol “ [symbol] ” means a single bond where the geometry around a double bond (e.g., either E or Z) is undefined. Both options, as well as combinations thereof are therefore intended. Any undefined valency on an atom of a structure shown in this application implicitly represents a hydrogen atom bonded to that atom. A bold dot on a carbon atom indicates that the hydrogen attached to that carbon is oriented out of the plane of the paper.

When a group “R” is depicted as a “floating group” on a ring system, for example, in the formula:

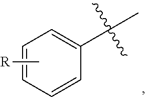

then R may replace any hydrogen atom attached to any of the ring atoms, including a depicted, implied, or expressly defined hydrogen, so long as a stable structure is formed. When a group “R” is depicted as a “floating group” on a fused ring system, as for example in the formula:

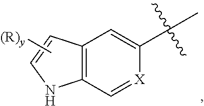

then R may replace any hydrogen attached to any of the ring atoms of either of the fused rings unless specified otherwise. Replaceable hydrogens include depicted hydrogens (e.g., the hydrogen attached to the nitrogen in the formula above), implied hydrogens (e.g., a hydrogen of the formula above that is not shown but understood to be present), expressly defined hydrogens, and optional hydrogens whose presence depends on the identity of a ring atom (e.g., a hydrogen attached to group X, when X equals —CH—), so long as a stable structure is formed. In the example depicted, R may reside on either the 5-membered or the 6-membered ring of the fused ring system. In the formula above, the subscript letter “y” immediately following the group “R” enclosed in parentheses, represents a numeric variable. Unless specified otherwise, this variable can be 0, 1, 2, or any integer greater than 2, only limited by the maximum number of replaceable hydrogen atoms of the ring or ring system.

For the chemical groups and compound classes, the number of carbon atoms in the group or class is as indicated as follows: “Cn” defines the exact number (n) of carbon atoms in the group/class. “C≤n” defines the maximum number (n) of carbon atoms that can be in the group/class, with the minimum number as small as possible for the group/class in question, e.g., it is understood that the minimum number of carbon atoms in the group “alkenyl_{(C≤8)}” or the class “alkene_{(C≤8)}” is two. Compare with “alkoxy_{(C≤10)}”, which designates alkoxy groups having from 1 to 10 carbon atoms. “Cn-n′” defines both the minimum (n) and maximum number (n′) of carbon atoms in the group. Thus, “alkyl_{(C2-10)}” designates those alkyl groups having from 2 to 10 carbon atoms. These carbon number indicators may precede or follow the chemical groups or class it modifies and it may or may not be enclosed in parenthesis, without signifying any change in meaning. Thus, the terms “C5 olefin”, “C5-olefin”, “olefin_{(C5)}”, and “olefin_{C5}” are all synonymous. When any of the chemical groups or compound classes defined herein is modified by the term “substituted”, any carbon atom(s) in a moiety replacing a hydrogen atom is not counted. Thus methoxyhexyl, which has a total of seven carbon atoms, is an example of a substituted alkyl_{(C1-6)}.

The term “aliphatic group” or “aliphatic” refers to a non-aromatic moiety that may be saturated (e.g. single bond) or contain one or more units of unsaturation, e.g., double and/or triple bonds. An aliphatic group may be straight chained, branched or cyclic, contain carbon, hydrogen or, optionally, one or more heteroatoms and may be substituted or unsubstituted. In addition to aliphatic hydrocarbon groups, aliphatic groups include, for example, polyalkoxyalkyls, such as polyalkylene glycols, polyamines, and polyimines, for example. Such aliphatic groups may be further substituted. It is understood that aliphatic groups may include alkyl, substituted alkyl, alkenyl, substituted alkenyl, alkynyl, substituted alkynyl, and substituted or unsubstituted cycloalkyl groups as described herein.

The term “saturated” when used to modify a compound or chemical group means the compound or chemical group has no carbon-carbon double and no carbon-carbon triple bonds, except as noted below. When the term is used to modify an atom, it means that the atom is not part of any double or triple bond. In the case of substituted versions of saturated groups, one or more carbon oxygen double bond or a carbon nitrogen double bond may be present. And when such a bond is present, then carbon-carbon double bonds that may occur as part of keto-enol tautomerism or imine/enamine tautomerism are not precluded. When the term “saturated” is used to modify a solution of a substance, it means that no more of that substance can dissolve in that solution.

The term “aliphatic” when used without the “substituted” modifier signifies that the compound or chemical group so modified is an acyclic or cyclic, but non-aromatic hydrocarbon compound or group. In aliphatic compounds/groups, the carbon atoms can be joined together in straight chains, branched chains, or non-aromatic rings (alicyclic). Aliphatic compounds/groups can be saturated, that is joined by single carbon-carbon bonds (alkanes/alkyl), or unsaturated, with one or more carbon-carbon double bonds (alkenes/alkenyl) or with one or more carbon-carbon triple bonds (alkynes/alkynyl).

The term “aromatic” when used to modify a compound or a chemical group refers to a planar unsaturated ring of atoms with 4n+2 electrons in a fully conjugated cyclic π system.

The term “alkyl” when used without the “substituted” modifier refers to a monovalent saturated aliphatic group with a carbon atom as the point of attachment, a linear or branched acyclic structure, and no atoms other than carbon and hydrogen. The groups —CH₃ (Me), —CH₂CH₃ (Et), —CH₂CH₂CH₃ (n-Pr or propyl), —CH(CH₃)₂(i-Pr, ⁱPr or isopropyl), —CH₂CH₂CH₂CH₃ (n-Bu), —CH(CH₃)CH₂CH₃ (sec-butyl), —CH₂CH(CH₃)₂(isobutyl), —C(CH₃)₃(tert-butyl, t-butyl, t-Bu or ^{t}Bu), and —CH₂C(CH₃)₃(neo-pentyl) are non-limiting examples of alkyl groups. The term “alkanediyl” when used without the “substituted” modifier refers to a divalent saturated aliphatic group, with one or two saturated carbon atom(s) as the point(s) of attachment, a linear or branched acyclic structure, no carbon-carbon double or triple bonds, and no atoms other than carbon and hydrogen. The groups —CH₂-(methylene), —CH₂CH₂—, —CH₂C(CH₃)₂CH₂—, and —CH₂CH₂CH₂— are non-limiting examples of alkanediyl groups. The term “alkylidene” when used without the “substituted” modifier refers to the divalent group ═CRR′ in which R and R′ are independently hydrogen or alkyl. Non-limiting examples of alkylidene groups include: ═CH₂, ═CH(CH₂CH₃), and ═C(CH₃)₂. An “alkane” refers to the class of compounds having the formula H—R, wherein R is alkyl as this term is defined above. When any of these terms is used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. The following groups are non-limiting examples of substituted alkyl groups: —CH₂OH, —CH₂Cl, —CF₃, —CH₂CN, —CH₂C(O)OH, —CH₂C(O)OCH₃, —CH₂C(O)NH₂, —CH₂C(O)CH₃, —CH₂OCH₃, —CH₂OC(O)CH₃, —CH₂NH₂, —CH₂N(CH₃)₂, and —CH₂CH₂Cl. The term “haloalkyl” is a subset of substituted alkyl, in which the hydrogen atom replacement is limited to halo (i.e. —F, —Cl, —Br, or —I) such that no other atoms aside from carbon, hydrogen and halogen are present. The group, —CH₂Cl is a non-limiting example of a haloalkyl. The term “fluoroalkyl” is a subset of substituted alkyl, in which the hydrogen atom replacement is limited to fluoro such that no other atoms aside from carbon, hydrogen and fluorine are present. The groups —CH₂F, —CF₃, and —CH₂CF₃ are non-limiting examples of fluoroalkyl groups.

The term “cycloalkyl” when used without the “substituted” modifier refers to a monovalent saturated aliphatic group with a carbon atom as the point of attachment, said carbon atom forming part of one or more non-aromatic ring structures, no carbon-carbon double or triple bonds, and no atoms other than carbon and hydrogen. Non-limiting examples include: —CH(CH₂)₂(cyclopropyl), cyclobutyl, cyclopentyl, or cyclohexyl (Cy). The term “cycloalkanediyl” when used without the “substituted” modifier refers to a divalent saturated aliphatic group with two carbon atoms as points of attachment, no carbon-carbon double or triple bonds, and no atoms other than carbon and hydrogen. The group

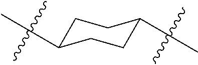 [SMILES: *C1CCC(*)CC1]

is a non-limiting example of cycloalkanediyl group. A “cycloalkane” refers to the class of compounds having the formula H—R, wherein R is cycloalkyl as this term is defined above. When any of these terms is used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “alkenyl” when used without the “substituted” modifier refers to an monovalent unsaturated aliphatic group with a carbon atom as the point of attachment, a linear or branched, acyclic structure, at least one nonaromatic carbon-carbon double bond, no carbon-carbon triple bonds, and no atoms other than carbon and hydrogen. Non-limiting examples include: —CH═CH₂ (vinyl), —CH═CHCH₃, —CH═CHCH₂CH₃, —CH₂CH═CH₂ (allyl), —CH₂CH═CHCH₃, and —CH═CHCH═CH₂. The term “alkenediyl” when used without the “substituted” modifier refers to a divalent unsaturated aliphatic group, with two carbon atoms as points of attachment, a linear or branched, a linear or branched acyclic structure, at least one nonaromatic carbon-carbon double bond, no carbon-carbon triple bonds, and no atoms other than carbon and hydrogen. The groups —CH═CH—, —CH═C(CH₃)CH₂—, —CH═CHCH₂—, and —CH₂CH═CHCH₂— are non-limiting examples of alkenediyl groups. It is noted that while the alkenediyl group is aliphatic, once connected at both ends, this group is not precluded from forming part of an aromatic structure. The terms “alkene” and “olefin” are synonymous and refer to the class of compounds having the formula H—R, wherein R is alkenyl as this term is defined above. Similarly the terms “terminal alkene” and “α-olefin” are synonymous and refer to an alkene having just one carbon-carbon double bond, wherein that bond is part of a vinyl group at an end of the molecule. When any of these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. The groups —CH═CHF, —CH═CHCl and —CH═CHBr are non-limiting examples of substituted alkenyl groups.

The term “aryl” when used without the “substituted” modifier refers to a monovalent unsaturated aromatic group with an aromatic carbon atom as the point of attachment, said carbon atom forming part of a one or more six-membered aromatic ring structure, wherein the ring atoms are all carbon, and wherein the group consists of no atoms other than carbon and hydrogen. If more than one ring is present, the rings may be fused or unfused. As used herein, the term does not preclude the presence of one or more alkyl or aralkyl groups (carbon number limitation permitting) attached to the first aromatic ring or any additional aromatic ring present. Non-limiting examples of aryl groups include phenyl (Ph), methylphenyl, (dimethyl)phenyl, —C₆H₄CH₂CH₃ (ethylphenyl), naphthyl, and a monovalent group derived from biphenyl. The term “arenediyl” when used without the “substituted” modifier refers to a divalent aromatic group with two aromatic carbon atoms as points of attachment, said carbon atoms forming part of one or more six-membered aromatic ring structure(s) wherein the ring atoms are all carbon, and wherein the monovalent group consists of no atoms other than carbon and hydrogen. As used herein, the term does not preclude the presence of one or more alkyl, aryl or aralkyl groups (carbon number limitation permitting) attached to the first aromatic ring or any additional aromatic ring present. If more than one ring is present, the rings may be fused or unfused. Unfused rings may be connected via one or more of the following: a covalent bond, alkanediyl, or alkenediyl groups (carbon number limitation permitting). Non-limiting examples of arenediyl groups include:

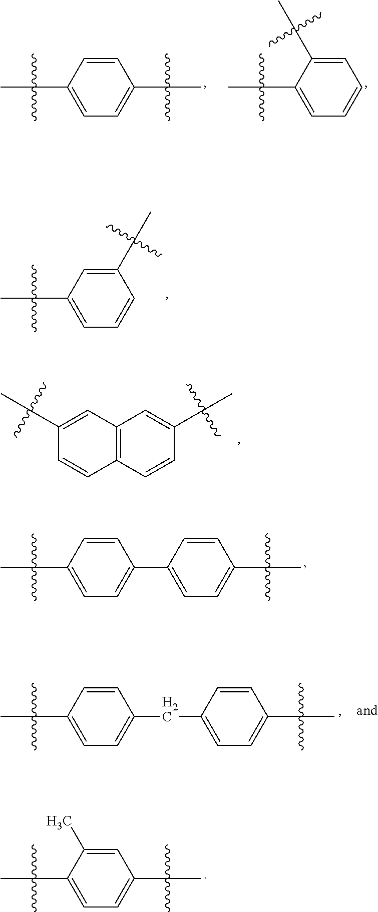

An “arene” refers to the class of compounds having the formula H—R, wherein R is aryl as that term is defined above. Benzene and toluene are non-limiting examples of arenes. When any of these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “aralkyl” when used without the “substituted” modifier refers to the monovalent group-alkanediyl-aryl, in which the terms alkanediyl and aryl are each used in a manner consistent with the definitions provided above. Non-limiting examples are: phenylmethyl (benzyl, Bn) and 2-phenyl-ethyl. When the term aralkyl is used with the “substituted” modifier one or more hydrogen atom from the alkanediyl and/or the aryl group has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. Non-limiting examples of substituted aralkyls are: (3-chlorophenyl)-methyl, and 2-chloro-2-phenyl-eth-1-yl.

The term “acyl” when used without the “substituted” modifier refers to the group —C(O)R, in which R is a hydrogen, alkyl, cycloalkyl, or aryl as those terms are defined above. The groups, —CHO, —C(O)CH₃ (acetyl, Ac), —C(O)CH₂CH₃, —C(O)CH(CH₃)₂, —C(O)CH(CH₂)₂, —C(O)C₆H₅, and —C(O)C₆H₄CH₃ are non-limiting examples of acyl groups. A “thioacyl” is defined in an analogous manner, except that the oxygen atom of the group —C(O)R has been replaced with a sulfur atom, —C(S)R. The term “aldehyde” corresponds to an alkyl group, as defined above, attached to a —CHO group. When any of these terms are used with the “substituted” modifier one or more hydrogen atom (including a hydrogen atom directly attached to the carbon atom of the carbonyl or thiocarbonyl group, if any) has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. The groups, —C(O)CH₂CF₃, —CO₂H (carboxyl), —CO₂CH₃ (methylcarboxyl), —CO₂CH₂CH₃, —C(O)NH₂ (carbamoyl), and —CON(CH₃)₂, are non-limiting examples of substituted acyl groups.

The term “alkoxy” when used without the “substituted” modifier refers to the group —OR, in which R is an alkyl, as that term is defined above. Non-limiting examples include: —OCH₃ (methoxy), —OCH₂CH₃ (ethoxy), —OCH₂CH₂CH₃, —OCH(CH₃)₂(isopropoxy), —OC(CH₃)₃(tert-butoxy), —OCH(CH₂)₂, —O-cyclopentyl, and —O-cyclohexyl. The terms “cycloalkoxy”, “alkenyloxy”, “aryloxy”, “aralkoxy”, and “acyloxy”, when used without the “substituted” modifier, refers to groups, defined as —OR, in which R is cycloalkyl, alkenyl, aryl, aralkyl, and acyl, respectively. The term “alkylthio” and “acylthio” when used without the “substituted” modifier refers to the group —SR, in which R is an alkyl and acyl, respectively. The term “alcohol” corresponds to an alkane, as defined above, wherein at least one of the hydrogen atoms has been replaced with a hydroxy group. The term “ether” corresponds to an alkane, as defined above, wherein at least one of the hydrogen atoms has been replaced with an alkoxy group. When any of these terms is used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “alkylamino” when used without the “substituted” modifier refers to the group —NHR, in which R is an alkyl, as that term is defined above. Non-limiting examples include: —NHCH₃ and —NHCH₂CH₃. The term “dialkylamino” when used without the “substituted” modifier refers to the group —NRR′, in which R and R′ can be the same or different alkyl groups, or R and R′ can be taken together to represent an alkanediyl. Non-limiting examples of dialkylamino groups include: —N(CH₃)₂ and —N(CH₃)(CH₂CH₃). The terms “cycloalkylamino”, “alkenylamino”, “arylamino”, “aralkylamino”, “alkoxyamino”, and “alkylsulfonylamino” when used without the “substituted” modifier, refers to groups, defined as —NHR, in which R is cycloalkyl, alkenyl, aryl, aralkyl, alkoxy, and alkylsulfonyl, respectively. A non-limiting example of an arylamino group is —NHC₆H₅. The term “amido” (acylamino), when used without the “substituted” modifier, refers to the group —NHR, in which R is acyl, as that term is defined above. A non-limiting example of an amido group is —NHC(O)CH₃. The term “alkylimino” when used without the “substituted” modifier refers to the divalent group ═NR, in which R is an alkyl, as that term is defined above. When any of these terms is used with the “substituted” modifier one or more hydrogen atom attached to a carbon atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. The groups —NHC(O)OCH₃ and —NHC(O)NHCH₃ are non-limiting examples of substituted amido groups.

The terms “alkylsulfonyl” and “alkylsulfinyl” when used without the “substituted” modifier refers to the groups —S(O)₂R and —S(O)R, respectively, in which R is an alkyl, as that term is defined above. The terms “cycloalkylsulfonyl”, “alkenylsulfonyl”, “alkynylsulfonyl”, “arylsulfonyl”, “aralkylsulfonyl”, “heteroarylsulfonyl”, and “heterocycloalkylsulfonyl” are defined in an analogous manner. When any of these terms is used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The use of the word “a” or “an,” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the inherent variation of error for the device, the method being employed to determine the value, or the variation that exists among the study subjects.

An “active ingredient” (AI) (also referred to as an active compound, active substance, active agent, pharmaceutical agent, agent, biologically active molecule, or a therapeutic compound) is the ingredient in a pharmaceutical drug or a pesticide that is biologically active. The similar terms active pharmaceutical ingredient (API) and bulk active are also used in medicine, and the term active substance may be used for pesticide formulations.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and also covers other unlisted steps. As used herein, the term “consisting substantially of” means that the composition contains at least 98% of the elements listed. Similarly, the term “consisting essentially of” means that the composition contains at least 99% of the elements listed.

The term “derivative” when used in the context of a protein, such as a cytokine or chemokine, refers to a protein which is a recombinant version, a fragment, a truncated version, or a chemically modified protein. In some aspects, the chemically modified protein is a protein to which a secondary group such as a PEG chain (pegylation) or other solubilizing polymer has been added to the protein.

The term “effective,” as that term is used in the specification and/or claims, means adequate to accomplish a desired, expected, or intended result. “Effective amount,” “Therapeutically effective amount” or “pharmaceutically effective amount” when used in the context of treating a patient or subject with a compound means that amount of the compound which, when administered to a subject or patient for treating or preventing a disease, is an amount sufficient to effect such treatment or prevention of the disease.

An “excipient” is a pharmaceutically acceptable substance formulated along with the active ingredient(s) of a medication, pharmaceutical composition, formulation, or drug delivery system. Excipients may be used, for example, to stabilize the composition, to bulk up the composition (thus often referred to as “bulking agents,” “fillers,” or “diluents” when used for this purpose), or to confer a therapeutic enhancement on the active ingredient in the final dosage form, such as facilitating drug absorption, reducing viscosity, or enhancing solubility. Excipients include pharmaceutically acceptable versions of antiadherents, binders, coatings, colors, disintegrants, flavors, glidants, lubricants, preservatives, sorbents, sweeteners, and vehicles. The main excipient that serves as a medium for conveying the active ingredient is usually called the vehicle. Excipients may also be used in the manufacturing process, for example, to aid in the handling of the active substance, such as by facilitating powder flowability or non-stick properties, in addition to aiding in vitro stability such as prevention of denaturation or aggregation over the expected shelf life. The suitability of an excipient will typically vary depending on the route of administration, the dosage form, the active ingredient, as well as other factors.

As used herein, “HSPCs” refers to hematopoietic stem and progenitor cells. HSPCs are a combination of progenitor cells and stem cells.

The term “hydrate” when used as a modifier to a compound means that the compound has less than one (e.g., hemihydrate), one (e.g., monohydrate), or more than one (e.g., dihydrate) water molecules associated with each compound molecule, such as in solid forms of the compound.

As used herein, the term “IC₅₀” refers to an inhibitory dose which is 50% of the maximum response obtained. This quantitative measure indicates how much of a particular active ingredient or other substance (inhibitor) is needed to inhibit a given biological, biochemical or chemical process (or component of a process, i.e. an enzyme, cell, cell receptor or microorganism) by half.

An “isomer” of a first compound is a separate compound in which each molecule contains the same constituent atoms as the first compound, but where the configuration of those atoms in three dimensions differs.

As used herein, the term “patient” or “subject” refers to a living organism, such as a human, monkey, cow, sheep, goat, dog, cat, mouse, rat, guinea pig, avain, or transgenic species thereof. In certain embodiments, the patient or subject is a primate. Non-limiting examples of human patients are adults, juveniles, infants and fetuses.

As generally used herein “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

“Pharmaceutically acceptable salts” means salts of compounds of the present invention which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as 1,2-ethanedisulfonic acid, 2-hydroxyethanesulfonic acid, 2-naphthalenesulfonic acid, 3-phenylpropionic acid, 4,4′-methylenebis(3-hydroxy-2-ene-1-carboxylic acid), 4-methylbicyclo[2.2.2]oct-2-ene-1-carboxylic acid, acetic acid, aliphatic mono- and dicarboxylic acids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hydroxybenzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tartaric acid, tertiarybutylacetic acid, trimethylacetic acid, and the like. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like. It should be recognized that the particular anion or cation forming a part of any salt of this invention is not critical, so long as the salt, as a whole, is pharmacologically acceptable. Additional examples of pharmaceutically acceptable salts and their methods of preparation and use are presented in Handbook of Pharmaceutical Salts: Properties, and Use (P. H. Stahl & C. G. Wermuth eds., Verlag Helvetica Chimica Acta, 2002).

A “pharmaceutically acceptable carrier,” “drug carrier,” or simply “carrier” is a pharmaceutically acceptable substance formulated along with the active ingredient medication that is involved in carrying, delivering and/or transporting a chemical agent. Drug carriers may be used to improve the delivery and the effectiveness of drugs, including for example, controlled-release technology to modulate drug bioavailability, decrease drug metabolism, and/or reduce drug toxicity. Some drug carriers may increase the effectiveness of drug delivery to the specific target sites. Examples of carriers include: liposomes, microspheres (e.g., made of poly(lactic-co-glycolic) acid), albumin microspheres, synthetic polymers, nanofibers, protein-DNA complexes, protein conjugates, erythrocytes, virosomes, and dendrimers.

A “pharmaceutical drug” (also referred to as a pharmaceutical, pharmaceutical preparation, pharmaceutical composition, pharmaceutical formulation, pharmaceutical product, medicinal product, medicine, medication, medicament, or simply a drug) is a compound or composition used to diagnose, cure, treat, or prevent disease. An active ingredient (AI) (defined above) is the ingredient in a pharmaceutical drug or a pesticide that is biologically active. The similar terms active pharmaceutical ingredient (API) and bulk active are also used in medicine, and the term active substance may be used for pesticide formulations. Some medications and pesticide products may contain more than one active ingredient. In contrast with the active ingredients, the inactive ingredients are usually called excipients (defined above) in pharmaceutical contexts.

As used herein, the term “pre-malignant cells” refers to cells that can form malignant hematopoietic or myeloid cells. The malignant hematopoietic or myeloid cells are those which characterize the conditions of myeloma, leukemia, and lymphoma. Particular forms of these diseases include acute myelitic leukemia (AML), acute lymphatic leukemia (ALL), multiple myeloma (MM), chronic myelogenous leukemia (CML), chronic lymphatic leukemia (CLL), hairy cell leukemia (HCL), acute promyelocytic leukemia (APL), and various lymphomas.

“Prevention” or “preventing” includes: (1) inhibiting the onset of a disease in a subject or patient which may be at risk and/or predisposed to the disease but does not yet experience or display any or all of the pathology or symptomatology of the disease, and/or (2) slowing the onset of the pathology or symptomatology of a disease in a subject or patient which may be at risk and/or predisposed to the disease but does not yet experience or display any or all of the pathology or symptomatology of the disease.

“Prodrug” means a compound that is convertible in vivo metabolically into an active ingredient according to the present invention. The prodrug itself may or may not also have activity with respect to a given target protein. For example, a compound comprising a hydroxy group may be administered as an ester that is converted by hydrolysis in vivo to the hydroxy compound. Suitable esters that may be converted in vivo into hydroxy compounds include acetates, citrates, lactates, phosphates, tartrates, malonates, oxalates, salicylates, propionates, succinates, fumarates, maleates, methylene-bis-β-hydroxynaphthoate, gentisates, isethionates, di-p-toluoyltartrates, methanesulfonates, ethanesulfonates, benzenesulfonates, p-toluenesulfonates, cyclohexylsulfamates, quinates, esters of amino acids, and the like. Similarly, a compound comprising an amine group may be administered as an amide that is converted by hydrolysis in vivo to the amine compound.

The term “progenitor cells” as used herein refers to cells that, in response to certain stimuli, can form differentiated hematopoietic or myeloid cells. The presence of progenitor cells can be assessed by the ability of the cells in a sample to form colony-forming units of various types, including, for example, CFU-GM (colony-forming units, granulocyte-macrophage); CFU-GEMM (colony-forming units, multipotential); BFU-E (burst-forming units, erythroid); HPP-CFC (high proliferative potential colony-forming cells); or other types of differentiated colonies which can be obtained in culture using known protocols such as those described below.

A “stereoisomer” or “optical isomer” is an isomer of a given compound in which the same atoms are bonded to the same other atoms, but where the configuration of those atoms in three dimensions differs. “Enantiomers” are stereoisomers of a given compound that are mirror images of each other, like left and right hands. “Diastereomers” are stereoisomers of a given compound that are not enantiomers. Chiral molecules contain a chiral center, also referred to as a stereocenter or stereogenic center, which is any point, though not necessarily an atom, in a molecule bearing groups such that an interchanging of any two groups leads to a stereoisomer. In organic compounds, the chiral center is typically a carbon, phosphorus or sulfur atom, though it is also possible for other atoms to be stereocenters in organic and inorganic compounds. A molecule can have multiple stereocenters, giving it many stereoisomers. In compounds whose stereoisomerism is due to tetrahedral stereogenic centers (e.g., tetrahedral carbon), the total number of hypothetically possible stereoisomers will not exceed 2ⁿ, where n is the number of tetrahedral stereocenters. Molecules with symmetry frequently have fewer than the maximum possible number of stereoisomers. A 50:50 mixture of enantiomers is referred to as a racemic mixture. Alternatively, a mixture of enantiomers can be enantiomerically enriched so that one enantiomer is present in an amount greater than 50%. Typically, enantiomers and/or diastereomers can be resolved or separated using techniques known in the art. It is contemplated that that for any stereocenter or axis of chirality for which stereochemistry has not been defined, that stereocenter or axis of chirality can be present in its R form, S form, or as a mixture of the R and S forms, including racemic and non-racemic mixtures. As used herein, the phrase “substantially free from other stereoisomers” means that the composition contains ≤15%, more preferably ≤10%, even more preferably ≤5%, or most preferably ≤1% of another stereoisomer(s).

“Stem cells”, as used herein, are less differentiated forms of progenitor cells. Typically, such cells are positive for CD34, but stem cells do not have to contain this marker. While other types of cells such as endothelial cells and mast cells also may exhibit this marker, CD34 is considered an one marker of stem cell presence. CD34+ cells can be assayed using fluorescence activated cell sorting (FACS) and thus their presence can be assessed in a sample using this technique. In general, CD34+ cells are present only in low levels in the blood, but are present in large numbers in bone marrow. Additionally, the stem cells may be hematopoietic stem cells that express the SLAM and LSK markers. Specifically, hematopoietic stem cells may be LSK cells or LSK-SLAM cells, which are considered early hematopoietic stem cells. The nomenclature, LSK, refers to Lin⁻Sca1⁺c-Kit⁺ and SLAM is signaling lymphocyte activation molecules or Lin⁻CD41⁻CD48⁻CD150⁺.

“Treatment” or “treating” includes (1) inhibiting a disease in a subject or patient experiencing or displaying the pathology or symptomatology of the disease (e.g., arresting further development of the pathology and/or symptomatology), (2) ameliorating a disease in a subject or patient that is experiencing or displaying the pathology or symptomatology of the disease (e.g., reversing the pathology and/or symptomatology), and/or (3) effecting any measurable decrease in a disease in a subject or patient that is experiencing or displaying the pathology or symptomatology of the disease.

The terms “VLA-4 antagonists” and “VLA-4 inhibitors” are used interchangeably herein.

The above definitions supersede any conflicting definition in any of the reference that is incorporated by reference herein. The fact that certain terms are defined, however, should not be considered as indicative that any term that is undefined is indefinite. Rather, all terms used are believed to describe the disclosure in terms such that one of ordinary skill can appreciate the scope and practice the present disclosure.

### EXAMPLES

The following examples are included to demonstrate various embodiments of the present disclosure. It should be appreciated by those of skill in the art that the techniques disclosed in the examples that follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1 (Section I. Integrin Antagonists)

(a) Instrumentation and General Methods.

Commercially available reagents and solvents were used without further purification unless stated otherwise. LC-MS analyses were performed on an Agilent 1100 or 1200 HPLC/MSD electrospray mass spectrometer in positive ion mode with scan range was 100-1000 d. Preparative normal phase chromatography was performed on a CombiFlash Rf+ (Teledyne Isco) with pre-packed RediSep Rf silica gel cartridges. Preparative reverse phase HPLC was performed on a CombiFlash Rf+(Teledyne Isco) equipped with RediSep Rf Gold pre-packed C18 cartridges and an acetonitrile/water/0.05% TFA gradient. The purity of tested compounds was 295% as determined by HPLC analysis conducted on an Agilent 1100 or 1260 system using a reverse phase C18 column with diode array detector unless stated otherwise. NMR spectra were recorded on a Bruker 400 MHz spectrometer. The signal of the deuterated solvent was used as internal reference. Chemical shifts (δ) are given in ppm and are referenced to residual not fully deuterated solvent signal. Coupling constants (J) are given in Hz.

(b) Preparation of Compounds

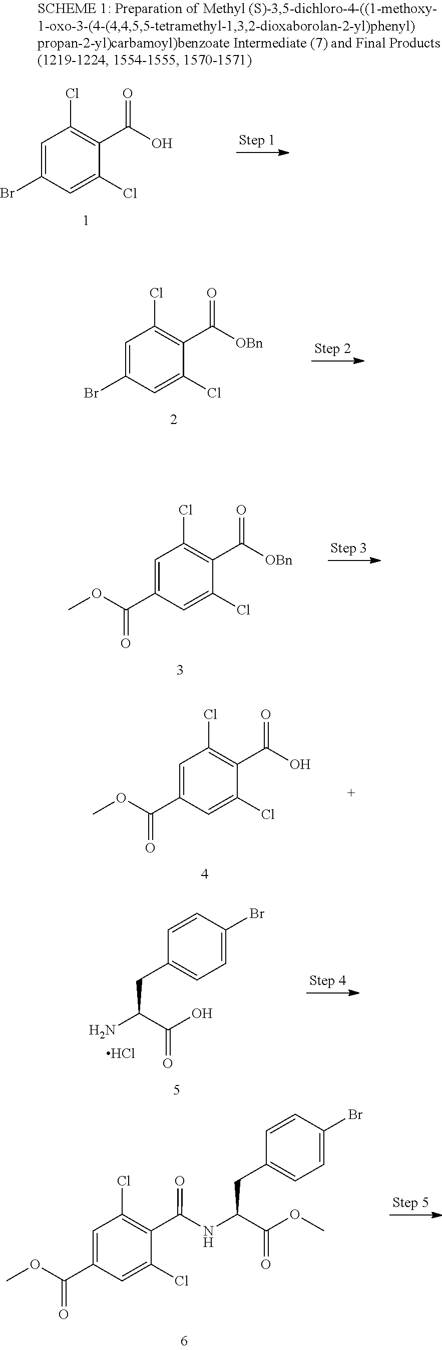

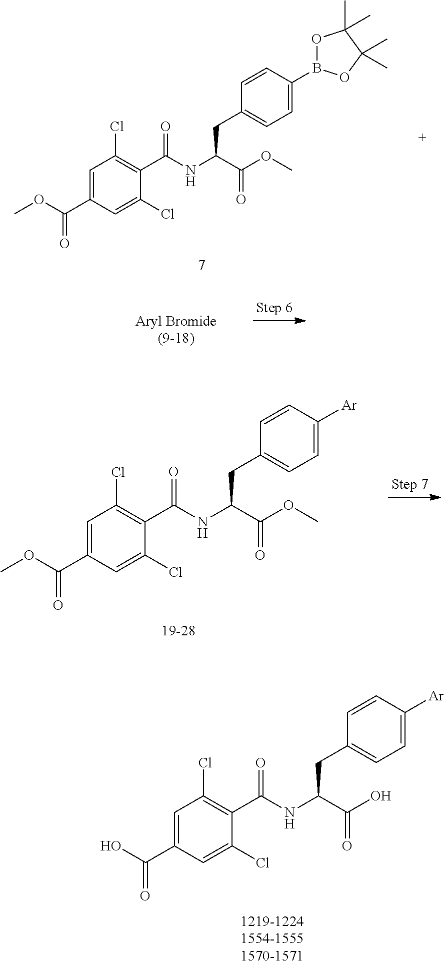

### Scheme 1, Step 1. Preparation of Benzyl 4-bromo-2,6-dichlorobenzoate (2)

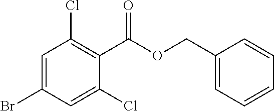 [SMILES: O=C(OCc1ccccc1)c1c(Cl)cc(Br)cc1Cl]

4-Bromo-2,6-dichlorobenzoic acid (Compound 1, 3.58 g, 13.3 mmol) and cesium carbonate (7.56 g, 23.2 mmol) were suspended in acetonitrile (50 mL) at 0° C. and benzyl bromide (2.38 g, 13.93 mmol) was added drop wise. The reaction was heated for 4 hours at 60° C. and then the reaction was cooled to room temperature and solids were filtered off and rinsed using additional acetonitrile. The filtrate was concentrated in vacuo and purified by chromatography on silica gel, eluting with ethyl acetate/hexanes. Product, benzyl 4-bromo-2,6-dichlorobenzoate, (Compound 2, 4.8 g, 99% yield) was isolated as a clear oil. LC-MS: t_{R}=3.03 min; m+23=381, 382, 383, 385 (bromine/bis-chlorine isotope pattern). ¹H NMR (400 MHz, DMSO-d₆) δ ppm 7.95 (s, 2H) 7.35-7.51 (m, 5H) 5.42 (s, 2H).

### Scheme 1, Step 2. Preparation of 1-Benzyl 4-methyl 2,6-dichlorobenzene-1,4-dicarboxylate (3)

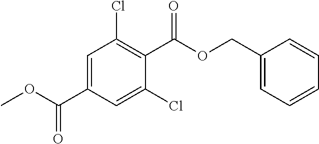 [SMILES: COC(=O)c1cc(Cl)c(C(=O)OCc2ccccc2)c(Cl)c1]

To a 20 mL microwave vial was added benzyl 4-bromo-2,6-dichlorobenzoate (Compound 2, 1.5 g, 4.17 mmol), palladium acetate (0.047 g, 0.208 mmol), 4,5-Bis(diphenylphosphino)-9,9-dimethylxanthene (0.241 g, 0.417 mmol), 4-dimethylaminopyridine (2 g, 16.7 mmol), Octacarbonyldicobalt (0.60 g, 3.33 mmol) and toluene/methanol (2:1, 15 mL). The vial was crimped shut and irradiated at 90° C. for 30 minutes using microwaves. The reaction was diluted with ethyl acetate, filtered through Celite® and concentrated in vacuo. The residue was taken up in ethyl acetate and washed using a 10% citric acid solution, then brine. The ethyl acetate layer was dried over sodium sulfate, filtered and concentrated in vacuo. The residue was purified by silica gel chromatography using ethyl acetate/hexanes as eluent to give product, 1-benzyl 4-methyl 2,6-dichlorobenzene-1,4-dicarboxylate, (Compound 3, 0.7 g, 81% yield) as a clear oil. LC-MS: t_{R}=2.88 min; m+23=361, 363 (chlorine isotope).

### Scheme 1, Step 3. Preparation of 2,6-Dichloro-4-(methoxycarbonyl)benzoic Acid (4)

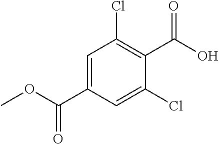

To a solution of 1-benzyl 4-methyl 2,6-dichlorobenzene-1,4-dicarboxylate (Compound 3, 2.3 g, 6.78 mmol) in ethyl acetate (20 mL) was added 10% palladium on carbon (0.35 g, 0.34 mmol). The mixture was stirred at room temperature under a hydrogen atmosphere at ambient pressure for 1.5 hours. The reaction was filtered through Celite® and concentrated in vacuo to give product, 2,6-dichloro-4-(methoxycarbonyl)benzoic acid, (Compound 4, 1.69 g, quantitative yield) as a crystalline solid. LC-MS: t_{R}=l 0.65 min; m+H=249, 251 (chlorine isotope).

### Scheme 1, Step 4. Preparation of methyl (S)-4-((3-(4-bromophenyl)-1-methoxy-1-oxopropan-2-yl)carbamoyl)-3,5-dichlorobenzoate (6)

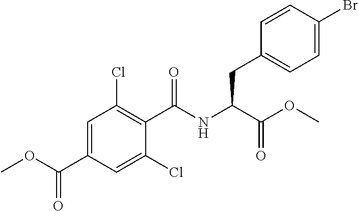 [SMILES: COC(=O)c1cc(Cl)c(C(=O)N[C@@H](Cc2ccc(Br)cc2)C(=O)OC)c(Cl)c1]

To a round bottom flask was added 2,6-dichloro-4-(methoxycarbonyl)benzoic acid (Compound 4, 2.08 g, 8.38 mmol), benzotriazol-1-ol (0.25 g, 1.59 mmol), 3-[Bis(dimethylamino)-methyliumyl]-3H-benzotriazol-1-oxide hexafluorophosphate (3.33 g, 8.77 mmol) and DMSO (15 mL). N,N-diisopropylethylamine (3.09 g, 23.93 mmol) was added and the reaction was stirred for 40 minutes at room temperature. At this time, methyl (S)-2-amino-3-(4-bromophenyl)propanoate hydrochloride (purchased from Ark Pharm) (Compound 5, 2.35 g, 7.98 mmol) was added and the reaction was stirred overnight at room temperature. The reaction was diluted with water (50 mL), stirred for 20 minutes, then extracted using ethyl acetate (100 mL). The ethyl acetate layer was washed using additional water, dried using sodium sulfate and concentrated in vacuo. The resulting oil was purified on silica gel using ethyl acetate and hexanes as eluent. Product, methyl 4-[[(1 S)-1-[(4-bromophenyl)methyl]-2-methoxy-2-oxo-ethyl]carbamoyl]-3,5-dichloro-benzoate, (Compound 6, 3.42 g, 87% yield) was isolated as a white foam. LC-MS: t_{R}=2.61 min; m+H=488, 489, 490, 491, 492 (bromine/bis-chlorine isotope pattern). ¹H NMR (400 MHz, DMSO-d₆) δ ppm 9.33 (d, J=8.31 Hz, 1H) 7.91 (s, 2H) 7.45-7.54 (d, 2H) 7.26 (d, J=8.31 Hz, 2H) 4.80 (ddd, J=10.09, 8.25, 4.89 Hz, 1H) 3.89 (s, 3H) 3.68 (s, 3H) 3.16 (dd, J=14.06, 5.01 Hz, 1H) 2.93 (dd, J=14.06, 10.15 Hz, 1H).

### Scheme 1, Step 5. Preparation of methyl (S)-3,5-dichloro-4-((1-methoxy-1-oxo-3-(4-(4,4,5,5-tetramethyl-1,3,2-dioxaborolan-2-yl)phenyl)propan-2-yl)carbamoyl)benzoate (7)

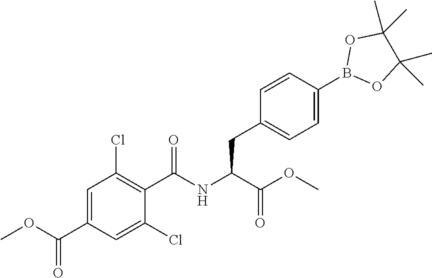

To a 20 mL microwave vial was added methyl 4-[[(1S)-1-[(4-bromophenyl)methyl]-2-methoxy-2-oxo-ethyl]carbamoyl]-3,5-dichloro-benzoate (Compound 6, 1 g, 2.04 mmole), Bis(pinacolato)diboron (0.675 g, 2.66 mmol), [1,1′-Bis(diphenylphosphino)ferrocene]-palladium(II) dichloride (0.100 g, 0.123 mmole, potassium acetate (0.6 g, 6.13 mmol) and 1,4-dioxane (10 mL). The vial was crimped shut, sparged for 10 minutes with nitrogen gas and then heated overnight at 80° C. The reaction was cooled to room temperature and filtered through Celite®, rinsing with ethyl acetate. The ethyl acetate layer was washed using additional water, dried using sodium sulfate and concentrated in vacuo. The resulting oil was purified on silica gel using ethyl acetate and hexanes as eluent. Product, methyl (S)-3,5-dichloro-4-((1-methoxy-1-oxo-3-(4-(4,4,5,5-tetramethyl-1,3,2-dioxaborolan-2-yl)phenyl)propan-2-yl)carbamoyl)benzoate, (Compound 7, 0.9 g, 82% yield) was isolated as a white foam. LC-MS: t_{R}=2.76 min; m+H=536.1, 538.0 (bis chlorine isotope pattern). ¹H NMR (400 MHz, DMSO-d₆) δ ppm 9.36 (d, J=8.07 Hz, 1H) 7.91 (d, J=0.49 Hz, 2H) 7.60 (d, J=7.58 Hz, 2H) 7.30 (d, J=7.58 Hz, 2H) 4.73-4.84 (m, 1H) 3.89 (d, J=0.49 Hz, 3H) 3.67 (s, 3H) 3.16 (dd, J=5.40 Hz, 1H) 3.01 (dd, J=9.80 Hz, 1H) 1.30 (s, 12H).

### Preparation of 4-Bromo-3,5-dimethoxybenzonitrile (9)

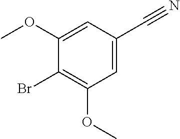

To a 50 mL 3-neck round bottom flask was added 4-bromo-3,5-dimethoxybenzoic acid (Compound 8, 2 g, 7.66 mmol), ammonium chloride (0.43 g, 8.04 mmol), triethylamine (1.2 mL, 8.27 mmol) and ethyl acetate (2 mL). The slurry was heated to 65° C. and 1-propanephosphonic acid cyclic anhydride (5.5 mL of a 50% solution in ethyl acetate, 9.19 mmol) was added over 20 minutes. Additional ethyl acetate was added and the heat was increased to 70° C. for 3 hours. The reaction was cooled and diluted using additional ethyl acetate and water. The aqueous layer was separated, filtered and rinsed using additional water. The isolated solid was dried overnight in vacuo. Product, 4-bromo-3,5-dimethoxybenzonitrile, (Compound 9, 0.929 g, 50% yield) was isolated as a white solid. LC-MS: t_{R}=2.937 min; m+H=242.9, 244.9. ¹H NMR (400 MHz, DMSO-d₆) δ ppm 7.42 (s, 2H) 3.96 (s, 6H).

### Preparation of (4-bromo-3,5-dimethoxyphenyl)methanol (10)

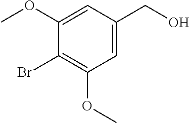

To an oven dried round bottom flask was added 4-bromo-3,5-dimethoxybenzoic acid (Compound 8, 2 g, 7.66 mmol) and anhydrous tetrahydrofuran (24 mL). Borane dimethylsulfide complex (7.6 mL of 2M in tetrahydrofuran, 15.3 mmol) was added drop-wise at room temperature. The reaction was heated overnight at 40° C. The reaction was quenched using hydrochloric acid (1N) and partitioned between ethyl acetate and water. The organic layer was washed using brine, dried with sodium sulfate, filtered and concentrated in vacuo. Product, (4-bromo-3,5-dimethoxyphenyl)methanol, (compound 10, 1.89 g, quantitative yield) was isolated as a white solid. LC-MS: t_{R}=1.748 min; m/z=229.0, 231.0 (dehydration). ¹H NMR (400 MHz, DMSO-d₆) δ ppm 6.71 (s, 2H) 4.49 (s, 2H) 3.82 (s, 6H).

### Preparation of 2-Bromo-5-(ethoxymethyl)-1,3-dimethoxybenzene (11)

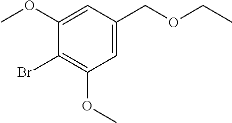

To a round bottom flask was added (4-bromo-3,5-dimethoxyphenyl)methanol (compound 10, 1.1 g, 4.45 mmol) and N,N-dimethylformamide (12 mL). The solution was cooled to 0° C. and sodium hydride (0.32 g of 60% (w/w) in mineral oil, 8.01 mmol) was added and the reaction was stirred for 1 hour at room temperature. Iodoethane (1 g, 6.68 mmol) was added and the reaction was stirred overnight at room temperature. The reaction was quenched with methanol and concentrated in vacuo. The residue was taken up in ethyl acetate and washed using water, then brine. The organic layer was dried using sodium sulfate, filtered and concentrated in vacuo. The resulting oil was purified on silica gel using ethyl acetate and hexanes as eluent. Product, 2-bromo-5-(ethoxymethyl)-1,3-dimethoxybenzene, (Compound 11, 0.306 g, 25% yield) was isolated as a clear oil. LC-MS: t_{R}=2.31 min; m/z=229.0, 231.0 (dehydration). ¹H NMR (400 MHz, DMSO-d₆) δ ppm 6.69 (s, 2H) 4.44 (s, 2H) 3.83 (s, 6H) 3.51 (q, J=7.09 Hz, 2H) 1.18 (t, J=6.97 Hz, 3H).

### Preparation of methyl 4-bromo-3,5-dimethoxybenzoate (12)

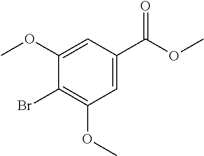

To a round bottom flask was added 4-bromo-3,5-dimethoxybenzoic acid (Compound 8, 4 g, 15.3 mmol) and methanol (100 mL). The solution was cooled to 0° C. and thionyl chloride (11.1 mL, 153 mmol) was slowly added. The reaction was heated to 80° C. for 2 hours. At this time the reaction was concentrated in vacuo and then taken up in ethyl acetate. The organic layer was washed with water, then brine. The ethyl acetate layer was dried using sodium sulfate, filtered and concentrated in vacuo. Product, methyl 4-bromo-3,5-dimethoxybenzoate, (Compound 12, 4.24 g, quantitative yield) was isolated as a crystalline white solid. LC-MS: t_{R}=2.3 min; m+H=274.9, 277.0 (bromine isotopes).

### Preparation of 2-(4-bromo-3,5-dimethoxyphenyl)propan-2-ol (13)

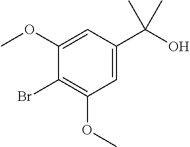

To a round bottom flask was added methyl 4-bromo-3,5-dimethoxybenzoate (Compound 12, 2 g, 7.27 mmol) and tetrahydrofuran (30 mL). The solution was cooled to −40° C. and methylmagnesium bromide (10 mL of a 3M solution in diethyl ether, 29 mmol) was added. The reaction was stirred at −40° C. for 1 hour and then gradually warmed to 0° C. The reaction was quenched using saturated ammonium chloride and extracted using ethyl acetate. The organic layer was washed with brine, dried using sodium sulfate, filtered and concentrated in vacuo. The resulting oil was purified on silica gel using ethyl acetate and hexanes as eluent. Product, 2-(4-bromo-3,5-dimethoxyphenyl)propan-2-ol, (Compound 13, 1.65 g, 82.5% yield) was isolated as a white solid. LC-MS: t_{R}=l 0.97 min; m/z=257.0, 259.0 (dehydration). ¹H NMR (400 MHz, DMSO-d₆) δ ppm 6.80 (s, 2H) 5.12 (s, 1H) 3.83 (s, 6H) 1.44 (s, 6H).

### Preparation of 2-bromo-5-(fluoromethyl)-1,3-dimethoxybenzene (14)

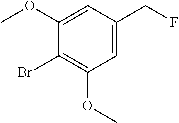

To a round bottom flask was added (4-bromo-3,5-dimethoxy-phenyl)methanol (Compound 10, 0.5 g, 2.02 mmol) and dichloromethane (15 mL). The reaction was cooled to 0° C. and diethylaminosulfur trifluoride (0.360 g, 2.23 mmol) was added. The reaction was allowed to warm to room temperature and stir for 1 hour. The reaction was quenched using saturated sodium bicarbonate, the layers were separated and the organic layer was dried using sodium sulfate, filtered and concentrated in vacuo. The resulting solid was purified on silica gel using ethyl acetate and hexanes as eluent. Product, 2-bromo-5-(fluoromethyl)-1,3-dimethoxybenzene, (Compound 14, 0.171 g, 33.9% yield) was isolated as a pale yellow solid. LC-MS: t_{R}=2.23 min; m+H=249.0, 251.0 (bromine isotopes).

### Preparation of 2-Bromo-1,3-dimethoxy-5-((2-methoxyethoxy)methyl)benzene (15)

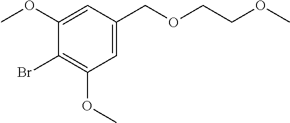

To a round bottom flask was added (4-bromo-3,5-dimethoxyphenyl)methanol (compound 10, 1.95 g, 7.89 mmol) and N,N-dimethylformamide (20 mL). The solution was cooled to 0° C. and sodium hydride (0.393 g of 60% (w/w) in mineral oil, 10.3 mmol) was added and the reaction was stirred for 1 hour at room temperature. 1-Bromo-2-methoxyethane (0.89 mL, 9.47 mmol) was added and the reaction was stirred overnight at room temperature. The reaction was quenched with methanol and concentrated in vacuo. The residue was taken up in ethyl acetate and washed using water, then brine. The organic layer was dried using sodium sulfate, filtered and concentrated in vacuo. The resulting oil was purified on silica gel using ethyl acetate and hexanes as eluent. Product 2-Bromo-1,3-dimethoxy-5-((2-methoxyethoxy)methy)benzene (15, 2.22 g, 92% yield) was isolated as a pale yellow oil. LC-MS: t_{R}=2.11 min; m/z+23=327.0, 329.0. ¹H NMR (400 MHz, DMSO-d6) δ ppm 6.70 (s, 2H) 4.48 (s, 2H) 3.82 (s, 6H) 3.53-3.62 (m, 2H) 3.45-3.52 (m, 2H) 3.26 (s, 3H).

### Preparation of 2-Bromo-5-((hexyloxy)methyl)-1,3-dimethoxybenzene (16)

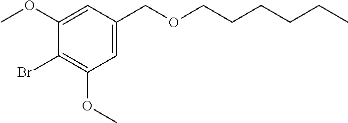 [SMILES: CCCCCCOCc1cc(OC)c(Br)c(OC)c1]

To a round bottom flask was added (4-bromo-3,5-dimethoxyphenyl)methanol (compound 10, 0.5 g, 2.02 mmol) and N,N-dimethylformamide (10 mL). The solution was cooled to 0° C. and sodium hydride (0.100 g of 60% (w/w) in mineral oil, 2.61 mmol) was added and the reaction was stirred for 1 hour at room temperature. 1-Bromohexane (0.34 mL, 2.43 mmol) was added and the reaction was stirred overnight at room temperature. The reaction was quenched with water and concentrated in vacuo. The residue was taken up in ethyl acetate and washed using water, then brine. The organic layer was dried using sodium sulfate, filtered and concentrated in vacuo. The resulting oil was purified on silica gel using ethyl acetate and hexanes as eluent. Product 2-Bromo-5-((hexyloxy)methyl)-1,3-dimethoxybenzene (16, 0.378 g, 56% yield) was isolated as a clear oil. LC-MS: t_{R}=2.98 min; m/z+23=353.0, 355.0. ¹H NMR (400 MHz, DMSO-d6) δ ppm 6.68 (s, 2H) 4.43 (s, 2H) 3.82 (s, 6H) 3.42 (t, J=6.48 Hz, 2H) 1.45-1.63 (m, 2H) 1.16-1.40 (m, 6H) 0.85 (t, J=1.00 Hz, 3H).

### Preparation of 1-(4-Bromo-3,5-dimethoxyphenyl-2,5,8,11-tetraoxadodecane (17)

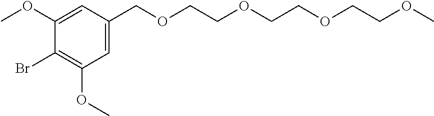

To a round bottom flask was added (4-bromo-3,5-dimethoxyphenyl)methanol (compound 10, 1.9 g, 7.69 mmol) and N,N-dimethylformamide (20 mL). The solution was cooled to 0° C. and sodium hydride (0.383 g of 60% (w/w) in mineral oil, 10 mmol) was added and the reaction was stirred for 1 hour at room temperature. 2-[2-(2-Methoxyethoxy)ethoxy]ethyl 4-methylbenzenesulfonate (2.8 mL, 9.23 mmol) was added and the reaction was stirred for six hours at room temperature. The reaction was quenched with water and concentrated in vacuo. The residue was taken up in ethyl acetate and washed using water, then brine. The organic layer was dried using sodium sulfate, filtered and concentrated in vacuo. The resulting oil was purified on silica gel using ethyl acetate and hexanes as eluent. Product 1-(4-Bromo-3,5-dimethoxyphenyl-2,5,8,11-tetraoxadodecane (17, 3.02 g, quant. yield) was isolated as a clear oil. LC-MS: t_{R}=2.0⁸ min; m/z+23=415.0, 417.0. ¹H NMR (400 MHz, DMSO-d6) € ppm 6.70 (s, 2H) 4.48 (s, 2H) 3.82 (s, 6H) 3.57 (s, 4H) 3.47-3.55 (m, 6H) 3.39-3.45 (m, 2H) 3.22 (s, 3H).

### Preparation of 4-Bromo-3,5-dimethoxybenzyl hexanoate (18)

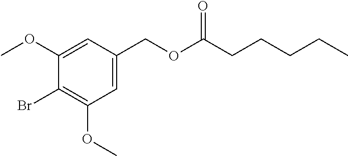 [SMILES: CCCCCC(=O)OCc1cc(OC)c(Br)c(OC)c1]

To a round bottom flask was added (4-bromo-3,5-dimethoxyphenyl)methanol (compound 10, 0.5 g, 2.02 mmol) and dichloromethane (10 mL). Triethylamine (0.56 mL, 4.05 mmol) and 4-dimethylaminopyridine (cat.) were added and the solution was cooled to 0° C. Hexanoyl chloride (0.37 mL, 2.63 mmol) was added and the reaction was stirred overnight at room temperature. The reaction was diluted with dichloromethane and washed using 1N HCl, then brine. The organic layer was dried using sodium sulfate, filtered and concentrated in vacuo. The resulting oil was purified on silica gel using ethyl acetate and hexanes as eluent. Product 4-Bromo-3,5-dimethoxybenzyl hexanoate (18, 0.630 g, 90% yield) was isolated as an off-white waxy solid. LC-MS: t_{R}=2.81 min; m/z+23=367.0, 369.0. ¹H NMR (400 MHz, DMSO-d6) δ ppm 6.74 (s, 2H) 5.06 (s, 2H) 3.83 (s, 6H) 2.38 (t, J=7.34 Hz, 2H) 1.49-1.62 (m, 2H) 1.20-1.33 (m, 4H) 0.82-0.89 (m, 3H).

### Scheme 1, Step 6: General Procedure A—Suzuki Coupling (Intermediates 19-28)

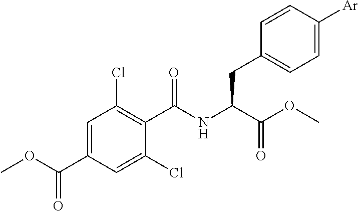

To a microwave vial was added methyl (S)-3,5-dichloro-4-((1-methoxy-1-oxo-3-(4-(4,4,5,5-tetramethyl-1,3,2-dioxaborolan-2-yl)phenyl)propan-2-yl)carbamoyl)benzoate (Compound 7, 100 mg, 0.186 mmol), aryl bromide coupling partner (Compounds 9-18, (0.28 mmol, 1.5 equivalent), tetrakis(triphenylphosphane)palladium(0) (11 mg, 0.009 mmol), cesium acetate (108 mg, 0.559 mmol), 1,4-dioxane (1 mL) and water (0.25 mL). The vial was crimped shut and sparged with nitrogen for 10 minutes and then heated overnight at 115° C. The reaction was partitioned between ethyl acetate and water, layers were separated and the ethyl acetate layer was dried using sodium sulfate, filtered and concentrated in vacuo. The residue was chromatographed on silica gel using ethyl acetate and hexanes as eluent. Isolated products (see Table 1, compounds 19-28) were carried forward as-is.

TABLE 2
Intermediates

| | | Pro- | | |
|---|---|---|---|---|
| S.M. | | duct | | LC/MS |
| # | S.M. Structure | # | Intermediate Structure | m/z |
| 9 | 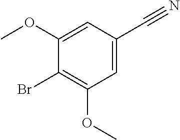 | 19 | 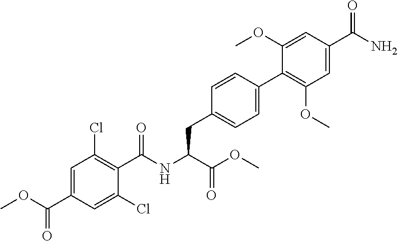 [SMILES: COC(=O)c1cc(Cl)c(C(=O)N[C@@H](Cc2ccc(-c3c(OC)cc(C(N)=O)cc3OC)cc2)C(=O)OC)c(Cl)c1] | 590.0, 592.0 |
| 10 | 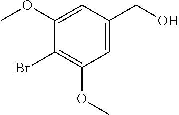 | 20 | 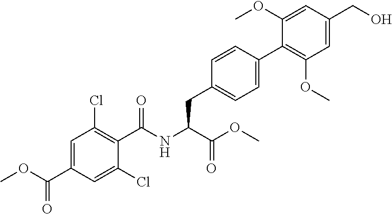 [SMILES: COC(=O)c1cc(Cl)c(C(=O)N[C@@H](Cc2ccc(-c3c(OC)cc(CO)cc3OC)cc2)C(=O)OC)c(Cl)c1] | 576.1, 578.0 |
| 11 | 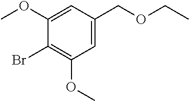 | 21 | 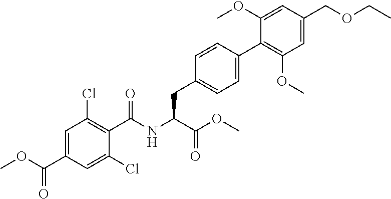 [SMILES: CCOCc1cc(OC)c(-c2ccc(C[C@H](NC(=O)c3c(Cl)cc(C(=O)OC)cc3Cl)C(=O)OC)cc2)c(OC)c1] | 604.1, 606.1 |
| 12 | 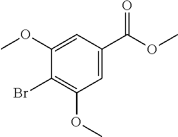 | 22 | 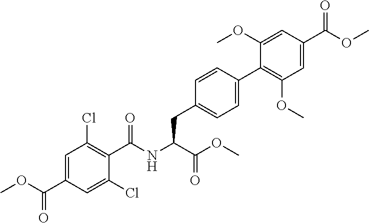 | 604.1, 606.0 |
| 13 | 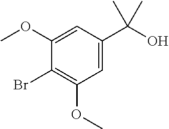 | 23 | 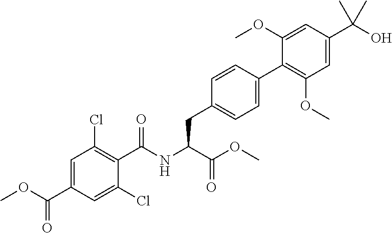 [SMILES: COC(=O)c1cc(Cl)c(C(=O)N[C@@H](Cc2ccc(-c3c(OC)cc(C(C)(C)O)cc3OC)cc2)C(=O)OC)c(Cl)c1] | 604.1, 606.1 |
| 14 | 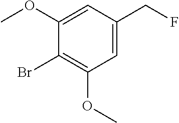 | 24 | 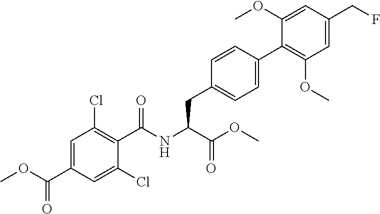 [SMILES: COC(=O)c1cc(Cl)c(C(=O)N[C@@H](Cc2ccc(-c3c(OC)cc(CF)cc3OC)cc2)C(=O)OC)c(Cl)c1] | 578.0, 580.0 |
| 15 | 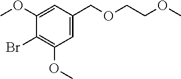 | 25 | 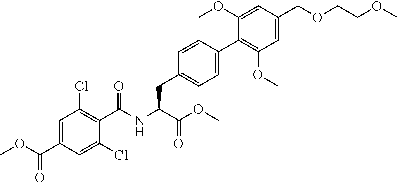 | 634.1, 636.1 |
| 16 | 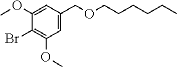 [SMILES: CCCCCCOCc1cc(OC)c(Br)c(OC)c1] | 26 | 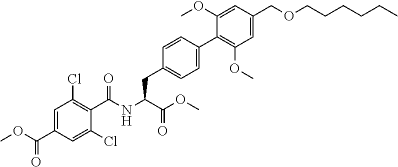 [SMILES: CCCCCCOCc1cc(OC)c(-c2ccc(C[C@H](NC(=O)c3c(Cl)cc(C(=O)OC)cc3Cl)C(=O)OC)cc2)c(OC)c1] | 660.1, 662.1 |
| 17 | 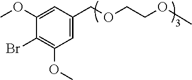 | 27 | 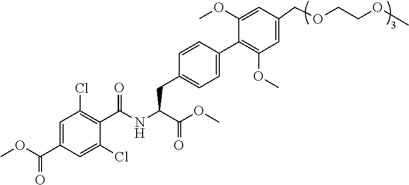 | 744.1, 746.1 (m + 23) |
| 18 | 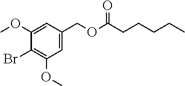 [SMILES: CCCCCC(=O)OCc1cc(OC)c(Br)c(OC)c1] | 28 | 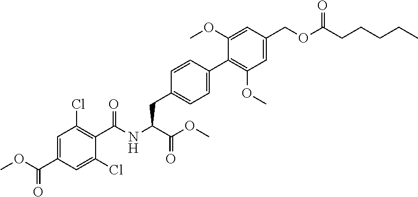 [SMILES: CCCCCC(=O)OCc1cc(OC)c(-c2ccc(C[C@H](NC(=O)c3c(Cl)cc(C(=O)OC)cc3Cl)C(=O)OC)cc2)c(OC)c1] | 674.1, 676.1 |

### Scheme 1, Step 7: General Procedure B—Hydrolysis (Products 1219-1224)

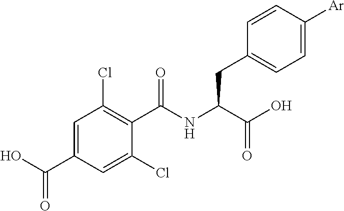

To a flask was added the appropriate bis ester intermediate (19-28) from Table 2, acetonitrile (1 mL), water (1 mL) and lithium hydroxide monohydrate (5 equivalents). The solution was stirred at room temperature for 2 hours. Acetonitrile was removed in vacuo and the reaction was acidified using hydrochloric acid (1N). Water was then removed in vacuo and the residue was purified on C18 using acetonitrile and water (both with 0.1% formic acid as modifier). Pure fractions were pooled and concentrated in vacuo and the resulting material was lyophilized from acetonitrile and water (1:4) overnight. Isolated products are shown in Table 3 (Products 1219-1224, 1554-1555, 1570-1571).

TABLE 3
Example Compounds and Characterization

| Ex. | | | | | LC/MS |
|---|---|---|---|---|---|
| No. | IUPAC Name | Structure | Yield | ¹H NMR δ | m/z |
| Cmpd 1219 | 4-{[(1S)-2-[4-(4- carbamoyl-2,6- dimethoxyphenyl) phenyl]-1-carboxy- ethyl]carbamoyl}- 3,5-dichlorobenzoic acid | 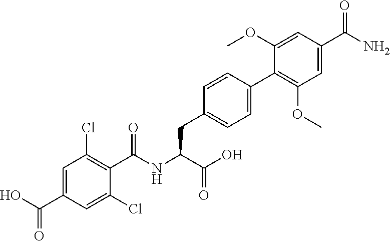 [SMILES: COc1cc(C(N)=O)cc(OC)c1-c1ccc(C[C@H](NC(=O)c2c(Cl)cc(C(=O)O)cc2Cl)C(=O)O)cc1] | 59% | 13.10 (br. s, 2 H) 9.20 (d, J = 8.31 Hz, 1 H) 7.83 (s, 2 H) 7.30 (d, J = 8.07 Hz, 2 H) 7.27 (s, 2 H) 7.13 (d, J = 8.07 Hz, 2 H) 4.72-4.82 (m, 1 H) 3.72 (s, 6 H) 3.33 (br. s, 2 H) 3.22 (dd, J = 13.94, 4.40 Hz, 1 H) 2.92 (dd, J = 14.06, 10.64 Hz, 1 H) | 562.0, 564.0 |
| Cmpd 1220 | 4-{[(1S)-1-carboxy- 2-{4-[4- (hydroxymethyl)- 2,6-dimethoxy- phenyl]phenyl} ethyl]carbamoyl}- 3,5-dichlorobenzoic acid | 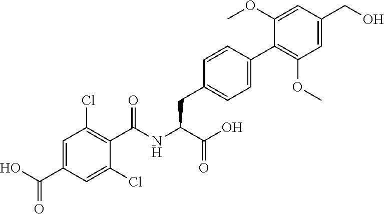 [SMILES: COc1cc(CO)cc(OC)c1-c1ccc(C[C@H](NC(=O)c2c(Cl)cc(C(=O)O)cc2Cl)C(=O)O)cc1] | 41% | 13.72 (br. s, 1 H) 12.90 (br. s, 1 H) 9.22 (d, J = 8.31 Hz, 1 H) 7.85 (s, 2 H) 7.26 (d, J = 8.07 Hz, 2 H) 7.08 (d, J = 8.07 Hz, 2 H) 6.69 (s, 2 H) 5.24 (br. s, 1 H) 4.66-4.82 (m, 1 H) 4.52 (s, 2 H) 3.65 (s, 6 H) 3.20 (dd, J = 14.18, 4.65 Hz, 1 H) 2.91 (dd, J = 14.31, 10.64 Hz, 1 H) | 548.0, 550.0 |
| Cmpd 1221 | 4-{[(1S)-1-carboxy- 2-{4-[4-(ethoxy- methyl)-2,6- dimethoxyphenyl] phenyl}ethyl] carbamoyl}-3,5- dichlorobenzoic acid | 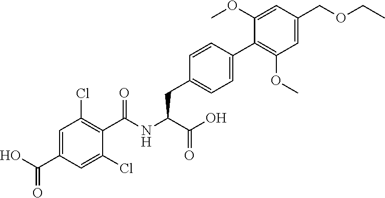 [SMILES: CCOCc1cc(OC)c(-c2ccc(C[C@H](NC(=O)c3c(Cl)cc(C(=O)O)cc3Cl)C(=O)O)cc2)c(OC)c1] | 52% | 13.74 (br. s, 1 H) 12.87 (br. s, 1 H) 9.22 (d, J = 8.31 Hz, 1 H) 7.85 (s, 2 H) 7.26 (d, J = 8.31 Hz, 2 H) 7.09 (d, J = 8.07 Hz, 2 H) 6.68 (s, 2 H) 4.76 (m, J = 2.20 Hz, 1 H) 4.47 (s, 2 H) 3.66 (s, 6 H) 3.53 (q, J = 7.09 Hz, 2 H) 3.20 (dd, J = 13.94, 4.40 Hz, 1 H) 2.91 (dd, J = 14.18, 10.27 Hz, 1 H) 1.19 (t, J = 6.97 Hz, 3 H) | 576.1, 578.0 |
| Cmpd 1222 | 4-{4-[(2S)-2- carboxy-2-[(4- carboxy-2,6- dichlorophenyl) formamido]ethyl] phenyl}-3,5- dimethoxy- benzoic acid | 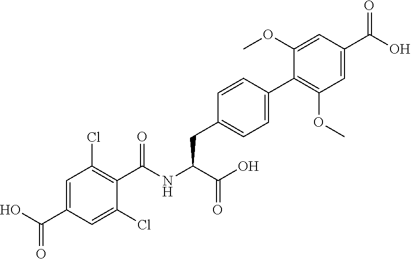 [SMILES: COc1cc(C(=O)O)cc(OC)c1-c1ccc(C[C@H](NC(=O)c2c(Cl)cc(C(=O)O)cc2Cl)C(=O)O)cc1] | 51% | 13.00 (br. s, 3 H) 9.22 (d, J = 8.31 Hz, 1 H) 7.85 (s, 2 H) 7.30 (d, J = 8.31 Hz, 2 H) 7.27 (s, 2 H) 7.13 (d, J = 8.07 Hz, 2 H) 4.71- 4.83 (m, 1 H) 3.72 (s, 6 H) 3.22 (dd, J = 14.31, 4.28 Hz, 1 H) 2.92 (dd, J = 13.94, 10.76 Hz, 1 H) | 562.0, 564.0 |
| Cmpd 1223 | 4-{[(1S)-1-carboxy- 2-{4-[4-(2- hydroxypropan-2- yl)-2,6-dimethoxy- phenyl]phenyl} ethyl]carbamoyl}- 3,5-dichlorobenzoic acid | 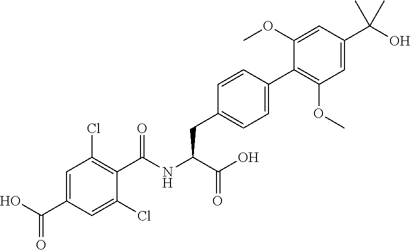 [SMILES: COc1cc(C(C)(C)O)cc(OC)c1-c1ccc(C[C@H](NC(=O)c2c(Cl)cc(C(=O)O)cc2Cl)C(=O)O)cc1] | 74% | 13.72 (br. s., 1 H) 12.88 (br. s., 1 H) 9.22 (d, J = 8.31 Hz, 1 H) 7.85 (s, 2 H) 7.25 (d, J = 8.07 Hz, 2 H) 7.09 (d, J = 8.31 Hz, 2 H) 6.81 (s, 2 H) 5.04 (s, 1 H) 4.67-4.82 (m, 1 H) 3.66 (s, 6 H) 3.20 (dd, J = 14.06, 4.03 Hz, 1 H) 2.91 (dd, J = 14.18, 10.51 Hz, 1 H) 1.47 (s, 6 H) | 576.1, 578.0 |
| Cmpd 1224 | 4-{[(1S)-1-carboxy- 2-{4-[4-(fluoro- methyl)-2,6- dimethoxyphenyl] phenyl}ethyl] carbamoyl}-3,5- dichlorobenzoic acid | 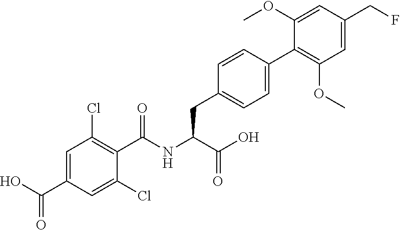 [SMILES: COc1cc(CF)cc(OC)c1-c1ccc(C[C@H](NC(=O)c2c(Cl)cc(C(=O)O)cc2Cl)C(=O)O)cc1] | 69% | 13.73 (br. s, 1 H) 12.89 (br. s, 1 H) 9.22 (d, J = 8.31 Hz, 1 H) 7.85 (s, 2 H) 7.28 (d, J = 8.07 Hz, 2 H) 7.10 (d, J = 8.07 Hz, 2 H) 6.80 (d, J = 1.47 Hz, 2 H) 5.49 (s, 1 H) 5.37 (s, 1 H) 4.77 (m, J = 2.40 Hz, 1 H) 3.68 (s, 6 H) 3.21 (dd, J = 14.18, 3.91 Hz, 1 H) 2.92 (dd, J = 14.06, 10.64 Hz, 1 H) | 550.0, 552.0 |
| Cmpd 1554 | (S)-4-((1-carboxy- 2-(2′,6′-dimethoxy- 4′-((2-methoxy- ethoxy)methyl)- [1,1′-biphenyl]-4- yl)ethyl) carbamoyl)-3,5- dichlorobenzoic acid | 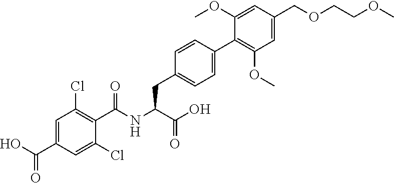 [SMILES: COCCOCc1cc(OC)c(-c2ccc(C[C@H](NC(=O)c3c(Cl)cc(C(=O)O)cc3Cl)C(=O)O)cc2)c(OC)c1] | 68% | ¹H NMR (400 MHz, DMSO-d6) δ ppm 13.74 (br. s, 1 H) 12.89 (br. s, 1 H) 9.23 (d, J = 8.31 Hz, 1 H) 7.85 (s, 2 H) 7.26 (d, J = 8.07 Hz, 2 H) 7.09 (d, J = 7.82 Hz, 2 H) 6.69 (s, 2 H) 4.70-4.83 (m, 1 H) 4.51 (s, 2 H) 3.66 (s, 6 H) 3.56-3.62 (m, 2 H) 3.49-3.55 (m, 2 H) 3.28 (s, 3 H) 3.20 (dd, J = 13.94, 3.91 Hz, | 606.0, 608.0 |
| | | | | 1 H) 2.91 (dd, J = 13.94, | |
| | | | | 10.76 Hz, 1 H) | |
| Cmpd 1555 | (S)-4-((1-carboxy- 2-(4′-((hexyloxy) methyl)-2′,6′- dimethoxy-[1,1′- biphenyl]-4-yl) ethyl)carbamoyl)- 3,5-dichloro- benzoic acid | 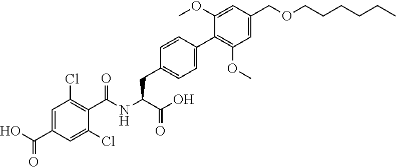 [SMILES: CCCCCCOCc1cc(OC)c(-c2ccc(C[C@H](NC(=O)c3c(Cl)cc(C(=O)O)cc3Cl)C(=O)O)cc2)c(OC)c1] | 48% | ¹H NMR (400 MHz, DMSO-d6) δ ppm 13.74 (br. s, 1 H) 12.89 (br. s, 1 H) 9.23 (d, J = 8.56 Hz, 1 H) 7.85 (s, 2 H) 7.26 (d, J = 8.07 Hz, 2 H) 7.09 (d, J = 8.07 Hz, 2 H) 6.68 (s, 2 H) 4.70-4.82 (m, 1 H) 4.47 (s, 2 H) 3.65 (s, 6 H) 3.46 (t, J = 6.48 Hz, 2 H) 3.14-3.26 (m, | 632.1, 634.1 |
| | | | | 1 H) 2.91 (dd, J = 13.82, | |
| | | | | 10.64 Hz, 1 H) 1.50- | |
| | | | | 1.65 (m, 2 H) 1.32-1.43 | |
| | | | | (m, 2 H) 1.22-1.31 (m, | |
| | | | | 4 H) 0.80-0.92 (m, 3 H) | |
| Cmpd 1570 | (S)-4-((2-(4′- (2,5,8,11- tetraoxadodecyl)- 2′,6′-dimethoxy- [1,1′-biphenyl]- 4-yl)-1- carboxyethyl) carbamoyl)-3,5- dichlorobenzoic acid | 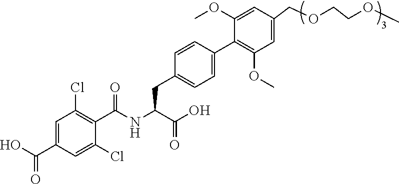 | 35% | ¹H NMR (400 MHz, DMSO-d6) δ ppm 12.98 (br. s, 2 H) 9.20 (d, J = 8.07 Hz, 1 H) 7.83 (s, 2 H) 7.26 (d, J = 8.07 Hz, 2 H) 7.09 (d, J = 8.07 Hz, 2 H) 6.69 (s, 2 H) 4.69-4.81 (m, 1 H) 4.52 (s, 2 H) 3.66 (s, 6 H) 3.60 (s, 4 H) 3.49-3.58 (m, 6 H) 3.40- 3.45 (m, 2 H) 3.23 (s, 3 H) 3.19 (dd, J = 1.00 | 716.1, 718.0 |
| | | | | Hz, 1 H) 2.91 (dd, J = | |
| | | | | 14.31, 10.39 Hz, 1 H) | |
| Cmpd 1571 | (S)-4-((1-carboxy- 2-(4′-((hexanoyl- oxy)methyl)- 2′,6′-dimethoxy- [1,1′-biphenyl]- 4-yl)ethyl) carbamoyl)-3,5- dichlorobenzoic acid | 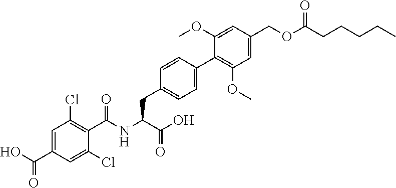 [SMILES: CCCCCC(=O)OCc1cc(OC)c(-c2ccc(C[C@H](NC(=O)c3c(Cl)cc(C(=O)O)cc3Cl)C(=O)O)cc2)c(OC)c1] | 56% | ¹H NMR (400 MHz, DMSO-d6) δ ppm 13.75 (br. s, 1 H) 12.91 (br. s, 1 H) 9.23 (d, J = 8.56 Hz, 1 H) 7.85 (s, 2 H) 7.27 (d, J = 8.07 Hz, 2 H) 7.09 (d, J = 8.07 Hz, 2 H) 6.73 (s, 2 H) 5.10 (s, 2 H) 4.76 (dd, J = 4.16, 1.96 Hz, 1 H) 3.66 (s, 6 H) 3.20 (dd, J = 14.18, 4.40 Hz, 1 H) 2.84-2.98 (m, 1 H) 2.39 (t, J = 7.34 Hz, 2 H) 1.58 (m, J = 7.20, 7.20 | 646.1, 648.0 |
| | | | | Hz, 2 H) 1.18-1.37 (m, | |
| | | | | 4 H) 0.86 (t, J = 1.00 Hz, | |
| | | | | 3 H) | |

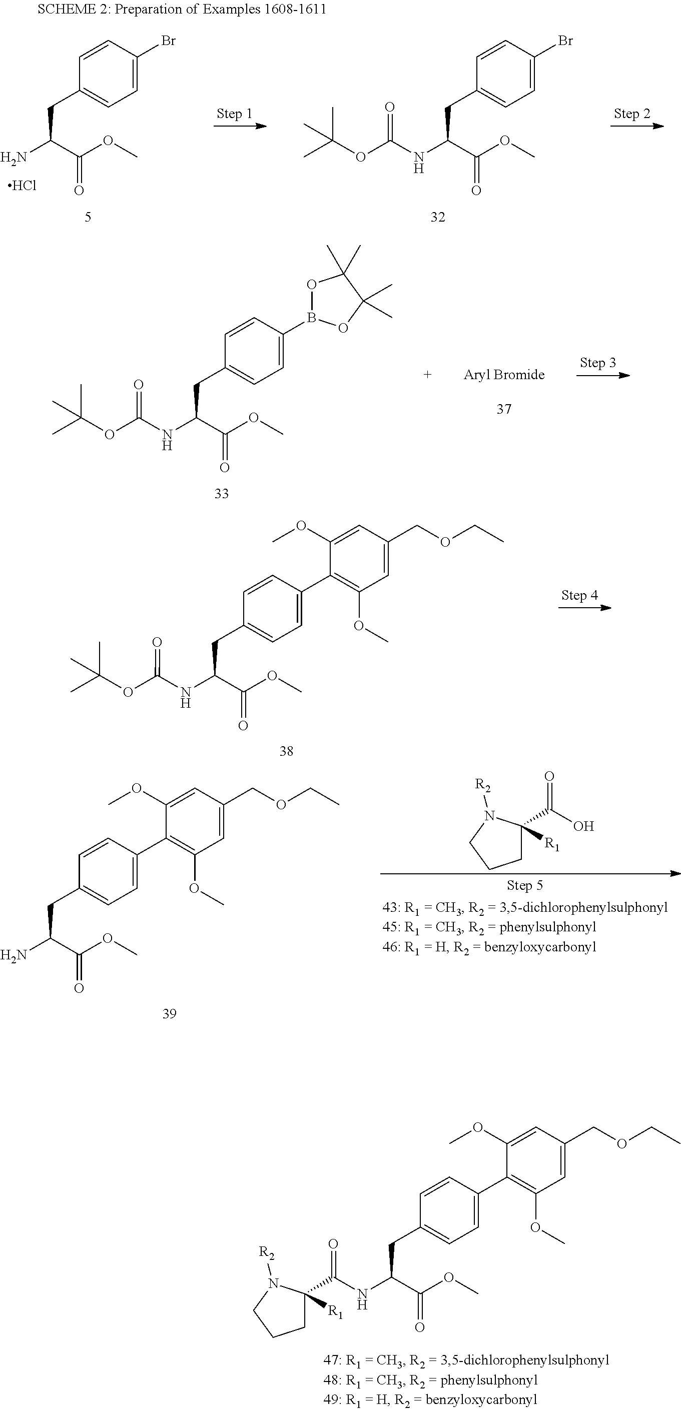

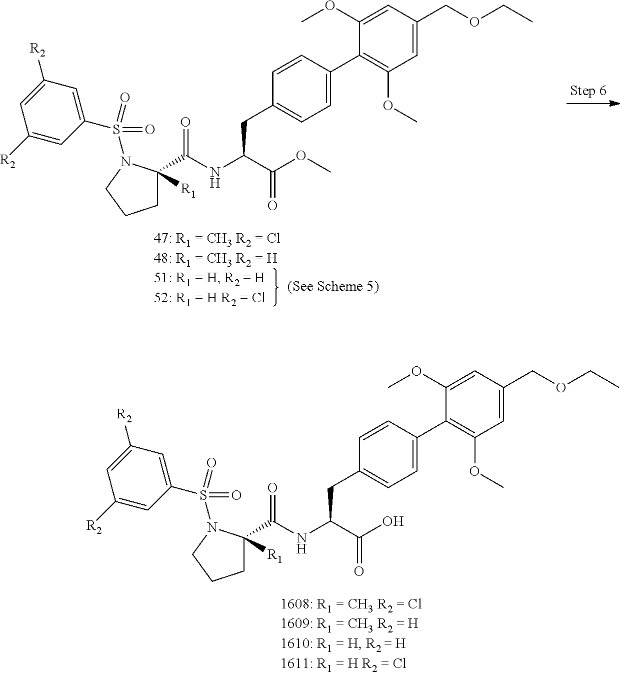

### Scheme 2, Step 1: Preparation of methyl (S)-3-(4-bromophenyl)-2-((tert-butoxycarbonyl) amino)propanoate (32)

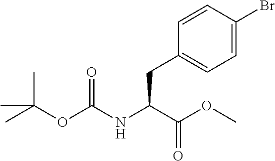 [SMILES: COC(=O)[C@H](Cc1ccc(Br)cc1)NC(=O)OC(C)(C)C]

To a round bottom flask was added methyl (2S)-2-amino-3-(4-bromophenyl) propanoate hydrochloride (compound 5, 3.35 g, 11.4 mmol), dichloromethane (40 mL) and triethylamine (3.2 mL, 22.7 mmol). The solution was cooled to 0° C. and di-tert-butyl dicarbonate (2.73 g, 12.5 mmol) was added. The reaction was stirred overnight at room temperature. Additional dichloromethane (200 mL) was added and the organic layer was washed using hydrochloric acid (50 mL, 1N), water, and then brine. The dichloromethane layer was dried over sodium sulfate, filtered and concentrated in vacuo to give product methyl (S)-3-(4-bromophenyl)-2-((tert-butoxycarbonyl)amino)propanoate (4.07 g, quant. yield) as a white, waxy solid. LC-MS: t_{R}=2.48 min; m/z+23=380.0, 382.0. ¹H NMR (400 MHz, DMSO-d6) δ ppm 1.32 (s, 9H) 2.82 (dd, J=13.69, 10.27 Hz, 1H) 2.94-3.02 (m, 1H) 3.62 (s, 3H) 4.13-4.21 (m, 1H) 7.20 (d, J=8.31 Hz, 2H) 7.31 (d, J=8.07 Hz, 1H) 7.48 (d, J=8.31 Hz, 2H).

### Scheme 2, Step 2: Preparation of methyl (S)-2-((tert-butoxycarbonyl)amino)-3-(4-(4,4,5,5-tetramethyl-1,3,2-dioxaborolan-2-yl)phenyl)propanoate (33)

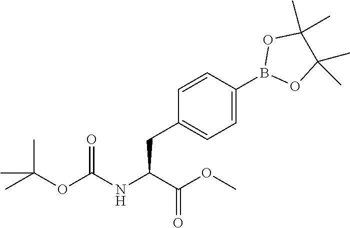 [SMILES: COC(=O)[C@H](Cc1ccc(B2OC(C)(C)C(C)(C)O2)cc1)NC(=O)OC(C)(C)C]

To a round bottom pressure vessel was added methyl (S)-3-(4-bromophenyl)-2-((tert-butoxycarbonyl)amino)propanoate (compound 32, 2 g, 5.58 mmol), bis(pinacolato) diboron (1.84 g, 7.26 mmol), Pd(dppf)C1₂ (0.245 g, 0.335 mmol), potassium acetate (1.64 g, 16.7 mmol) and 1,4-dioxane (20 mL). The vessel was sparged with nitrogen gas for 10 minutes, sealed, and heated overnight at 90 0C. The reaction was filtered through a plug of Celite® and rinsed with ethyl acetate. The organic layer was washed with water and brine, dried over sodium sulfate, then filtered and concentrated in vacuo. The resulting oil was purified on silica gel using ethyl acetate and hexanes as eluent. Product methyl (S)-2-((tert-butoxycarbonyl)amino)-3-(4-(4,4,5,5-tetramethyl-1,3,2-dioxaborolan-2-yl)phenyl)propanoate (2.26 g, quant. yield) was isolated as an oil. LC-MS: t_{R}=2.70 min; m/z+23=428.2.

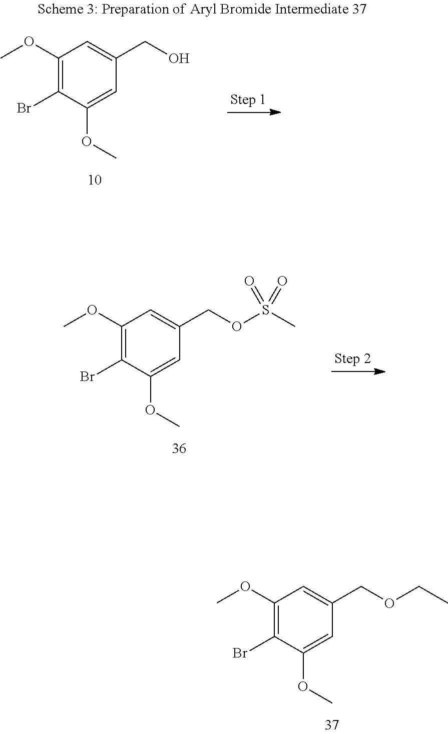

### Scheme 3, Step 1: Preparation of 4-bromo-3,5-dimethoxybenzyl methanesulfonate (36)

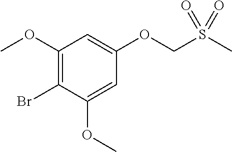

To a round bottom flask was added (4-bromo-3,5-dimethoxyphenyl)methanol (compound 10, 2.8 g, 11.3 mmol) and dichloromethane (50 mL). The solution was cooled to 0° C. and triethylamine (2 mL, 14.7 mmol) was added followed by methanesulfonyl chloride (1 mL, 12.5 mmol) and the reaction was stirred at room temperature. Upon completion, the reaction was diluted with additional dichloromethane and washed using dilute HCl, then brine. The organic layer was dried using sodium sulfate, filtered and concentrated in vacuo to give product 4-bromo-3,5-dimethoxybenzyl methanesulfonate (36, 3.68 g, quant. yield) as an oil. LC: t_{R}=2.12 min. The material was carried forward without further purification or characterization.

### Scheme 3, Step 2: Preparation of 2-bromo-5-(ethoxymethyl)-1,3-dimethoxybenzene (37)

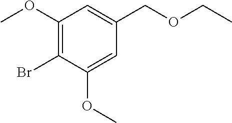

To a microwave vial was added 4-bromo-3,5-dimethoxybenzyl methane sulfonate (36, 3.68 g, 11.3 mmol) and ethanol (20 mL). Potassium carbonate (2.34 g, 16.9 mmol) was added followed by potassium iodide (catalytic) and the reaction was irradiated with microwaves at 140° C. for 2 hours. The reaction was filtered through a plug of Celite® and rinsed with additional ethanol. The solvent was removed in vacuo and the resulting oil was purified on silica gel using ethyl acetate and hexanes as eluent. Product 2-bromo-5-(ethoxymethyl)-1,3-dimethoxybenzene (37, 1.77 g, 57% yield) was isolated as an oil. LC: t_{R}=2.29 min. ¹H NMR (400 MHz, DMSO-d₆) δ ppm 6.69 (s, 2H) 4.44 (s, 2H) 3.83 (s, 6H) 3.51 (q, J=7.09 Hz, 2H) 1.18 (t, J=6.97 Hz, 3H).

### Scheme 2, Step 3: Preparation of methyl (S)-2-((tert-butoxycarbonyl)amino)-3-(4′-(ethoxymethyl)-2′,6′-dimethoxy-[1,1′-biphenyl]-4-yl)propanoate (38)

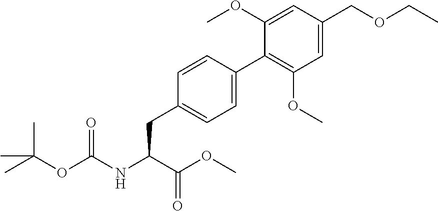 [SMILES: CCOCc1cc(OC)c(-c2ccc(C[C@H](NC(=O)OC(C)(C)C)C(=O)OC)cc2)c(OC)c1]

To a microwave vial was added methyl (S)-2-((tert-butoxycarbonyl)amino)-3-(4-(4,4,5,5-tetramethyl-1,3,2-dioxaborolan-2-yl)phenyl)propanoate (33, 1.26 g, 3.11 mmol), 2-bromo-5-(ethoxymethyl)-1,3-dimethoxybenzene (37, 1.26 g, 4.35 mmol), tetrakis (triphenylphosphane)palladium(0) (180 mg, 0.155 mmol), cesium acetate (1.79 g, 9.33 mmol), 1,4-dioxane (10 mL) and water (2 mL). The vial was crimped shut and sparged with nitrogen for 10 minutes and then heated for 48 hours at 120° C. The reaction was partitioned between ethyl acetate and water, layers were separated and the ethyl acetate layer was dried using sodium sulfate, filtered and concentrated in vacuo. The residue was chromatographed on silica gel using ethyl acetate and hexanes as eluent. Pure fractions were pooled and concentrated in vacuo to give product methyl (S)-2-((tert-butoxycarbonyl)amino)-3-(4′-(ethoxymethyl)-2′,6′-dimethoxy-[1,1′-biphenyl]-4-yl)propanoate (38, 0.72 g, 49% yield) as an oil. LC-MS: t_{R}=2.72 min; m/z+23=496.2.

### Scheme 2, Step 4: Preparation of methyl (S)-2-amino-3-(4′-(ethoxymethyl)-2′,6′-dimethoxy-[1,1′-biphenyl]-4-yl)propanoate (39)

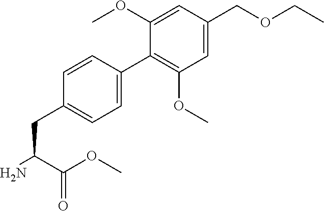 [SMILES: CCOCc1cc(OC)c(-c2ccc(C[C@H](N)C(=O)OC)cc2)c(OC)c1]

To a round bottom flask was added methyl (S)-2-((tert-butoxycarbonyl)amino)-3-(4′-(ethoxymethyl)-2′,6′-dimethoxy-[1,1′-biphenyl]-4-yl)propanoate (38, 0.955 g, 2.02 mmol) and trifluoroacetic acid in dichloromethane (10 mL, 20% v/v) and the reaction was stirred overnight at room temperature. The reaction was concentrated in vacuo and then partitioned between ethyl acetate and saturated sodium bicarbonate, layers were separated and the ethyl acetate layer was dried using sodium sulfate, filtered and concentrated in vacuo. The residue was chromatographed on silica gel using methanol and dichloromethane as eluent. Pure fractions were pooled and concentrated in vacuo to give product methyl (S)-2-amino-3-(4′-(ethoxymethyl)-2′,6′-dimethoxy-[1,1′-biphenyl]-4-yl) propanoate (39, 0.6 g, 80% yield) as an oil. LC-MS: t_{R}=1.96 min; m/z=374.2. ¹H NMR (400 MHz, DMSO-d6) δ ppm 7.15 (d, J=1.00 Hz, 2H) 7.10 (d, J=1.00 Hz, 2H) 6.68 (s, 2H) 4.47 (s, 2H) 3.65 (s, 6H) 3.61 (s, 3H) 3.59 (t, J=1.00 Hz, 1H) 3.53 (q, J=7.01 Hz, 2H) 2.91 (dd, J=13.69, 5.87 Hz, 1H) 2.75 (dd, J=13.45, 7.58 Hz, 1H) 1.80 (br. s., 2H) 1.19 (t, J=6.97 Hz, 3H).

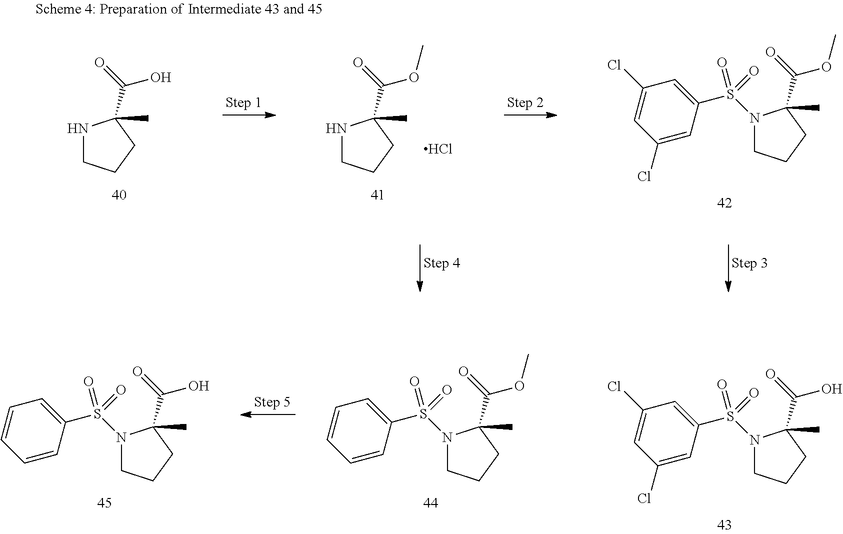

### Scheme 4, Step 1: Preparation of methyl (S)-2-methylpyrrolidine-2-carboxylate Hydrochloride (41)

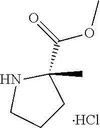

To a round bottom flask was added (S)-2-methylpyrrolidine-2-carboxylic acid (40, purchased from TCl, 1.75 g, 13.5 mmol) and methanol (100 mL). The solution was cooled to 0° C., thionyl chloride (25 mL, 340 mmol) was slowly added and the reaction was refluxed overnight. The reaction was concentrated in vacuo, repeatedly evaporated from methanol (4×50 mL), and then pulled overnight under a high vacuum. Product methyl (S)-2-methylpyrrolidine-2-carboxylate hydrochloride (41, 2.43 g, quant. yield) was isolated as a solid. ¹H NMR (400 MHz, DMSO-d6) δ ppm 1.59 (s, 3H) 1.82-2.09 (m, 3H) 2.14-2.37 (m, 1H) 3.78 (s, 3H) 3.88-4.48 (m, 2H) 9.36 (br. s, 1H) 10.24 (br. s, 1H).

### Scheme 4, Step 2: Preparation of methyl (S)-1-((3,5-dichlorophenyl)sulfonyl)-2-methylpyrrolidine-2-carboxylate (42)

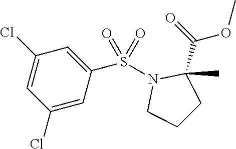 [SMILES: COC(=O)[C@]1(C)CCCN1S(=O)(=O)c1cc(Cl)cc(Cl)c1]

To a round bottom flask was added methyl (S)-2-methylpyrrolidine-2-carboxylate hydrochloride (41, 1.28 g, 7.13 mmol) and dichloromethane (10 mL). The reaction was cooled to 0° C. and 3,5-dichlorobenzenesulfonyl chloride (3.5 g, 14.3 mmol) was slowly added. Diisopropylethylamine (4.6 g, 35.6 mmol) was added and the reaction was stirred overnight at room temperature. The reaction was taken up in ethyl acetate and washed with HCl (1N), and then brine. The layers were separated and the ethyl acetate layer was dried using sodium sulfate, filtered and concentrated in vacuo. The residue was chromatographed on silica gel using ethyl acetate and hexanes as eluent. Pure fractions were pooled and concentrated in vacuo to give product methyl (S)-1-((3,5-dichlorophenyl)sulfonyl)-2-methylpyrrolidine-2-carboxylate (42, 2.21 g, 88% yield) as a crystalline solid. LC-MS: t_{R}=2.61 min; m/z=352.0, 354.0. ¹H NMR (400 MHz, DMSO-d6) δ ppm 1.53 (s, 3H) 1.84-2.05 (m, 3H) 2.05-2.23 (m, 1H) 3.36-3.56 (m, 2H) 3.64 (s, 3H) 7.78 (d, J=1.71 Hz, 2H) 8.00 (t, J=1.83 Hz, 1H).

### Scheme 4, Step 3: Preparation of (S)-1-((3,5-dichlorophenyl)sulfonyl)-2-methylpyrrolidine-2-carboxylic Acid (43)

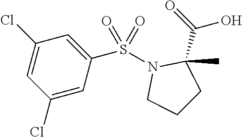 [SMILES: C[C@@]1(C(=O)O)CCCN1S(=O)(=O)c1cc(Cl)cc(Cl)c1]

To a round bottom flask was added methyl (S)-1-((3,5-dichlorophenyl)sulfonyl)-2-methylpyrrolidine-2-carboxylate (42, 2.1 g, 5.96 mmol), acetonitrile (5 mL), tetrahydrofuran (5 mL) and water (5 mL). Lithium hydroxide hydrate (0.5 g, 11.9 mmol) was added and the reaction was stirred overnight at room temperature. The organic solvents were evaporated and the remaining aqueous solution was adjusted to pH-5 with HCl (˜2.4 mL, 5N). The aqueous layer was extracted using ethyl acetate, washed using brine, dried with sodium sulfate, filtered and evaporated. Product (S)-1-((3,5-dichlorophenyl)sulfonyl)-2-methylpyrrolidine-2-carboxylic acid (43, 2.0 g, 99% yield) was isolated as an off-white solid. LC-MS: t_{R}=2.30 min; m/z=338.0, 339.9. ¹H NMR (400 MHz, DMSO-d6) δ ppm 1.52 (s, 3H) 1.85-2.01 (m, 3H) 2.07-2.21 (m, 1H) 3.35-3.51 (m, 2H) 7.79 (d, J=1.96 Hz, 2H) 7.97 (t, J=1.83 Hz, 1H) 12.91 (br. s., 1H).

### Scheme 4, Step 4: Preparation of methyl (S)-2-methyl-1-(phenylsulfonyl)pyrrolidine-2-carboxylate (44)

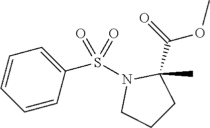 [SMILES: COC(=O)[C@]1(C)CCCN1S(=O)(=O)c1ccccc1]

To a round bottom flask was added methyl (S)-2-methylpyrrolidine-2-carboxylate hydrochloride (41, 1.24 g, 6.90 mmol) and dichloromethane (10 mL). The reaction was cooled to 0° C. and benzenesulfonyl chloride (2.44 g, 13.8 mmol) was slowly added. Diisopropylethylamine (4.46 g, 34.5 mmol) was added and the reaction was stirred overnight at room temperature. The reaction was taken up in ethyl acetate and washed with HCl (1N), and then brine. The layers were separated and the ethyl acetate layer was dried using sodium sulfate, filtered and concentrated in vacuo. The residue was chromatographed on silica gel using ethyl acetate and hexanes as eluent. Pure fractions were pooled and concentrated in vacuo to give product methyl (S)-2-methyl-1-(phenylsulfonyl)pyrrolidine-2-carboxylate (44, 1.52 g, 78% yield) as an oil. LC-MS: t_{R}=2.05 min; m/z=284.1. ¹H NMR (400 MHz, DMSO-d6) δ ppm 7.74-7.87 (m, 2H) 7.54-7.72 (m, 3H) 3.61 (s, 3H) 3.29-3.49 (m, 2H) 2.03-2.21 (m, 1H) 1.80-1.97 (m, 3H) 1.49 (s, 3H).

### Scheme 4, Step 5: Preparation of (S)-2-methyl-1-(phenylsulfonyl)pyrrolidine-2-carboxylic Acid (45)

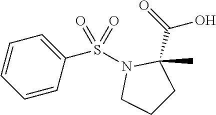 [SMILES: C[C@@]1(C(=O)O)CCCN1S(=O)(=O)c1ccccc1]

To a round bottom flask was added methyl (S)-2-methyl-1-(phenylsulfonyl)pyrrolidine-2-carboxylate (44, 1.52 g, 5.36 mmol), acetonitrile (5 mL), and water (5 mL). Lithium hydroxide hydrate (0.45 g, 10.7 mmol) was added and the reaction was stirred overnight at room temperature. The organic solvents were evaporated and the remaining aqueous solution was adjusted to pH-5 with HCl (˜8 mL, 2N). The remaining aqueous layer was extracted using ethyl acetate, washed using brine, dried with sodium sulfate, filtered and evaporated. Product (S)-2-methyl-1-(phenylsulfonyl)pyrrolidine-2-carboxylic acid (45, 1.25 g, 86% yield) was isolated as an off-white solid. LC-MS: t_{R}=1.78 min; m/z+23=292.0. ¹H NMR (400 MHz, DMSO-d6) b ppm 12.72 (s, 1H) 7.76-7.88 (m, 2H) 7.52-7.70 (m, 3H) 3.36-3.45 (m, 1H) 3.26-3.36 (m, 1H) 2.06-2.20 (m, 1H) 1.77-1.97 (m, 3H) 1.46 (s, 3H).

### Scheme 2, Step 5: General Procedure C—Amide Coupling (Intermediates 47-49)

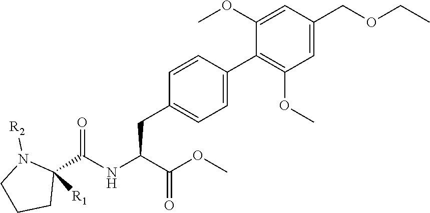

47: R₁═CH₃, R₂=3,5-dichlorophenylsulphonyl

48: R₁═CH₃, R₂=phenylsulphonyl

49: R₁═H, R₂=benzyloxycarbonyl

To a round bottom flask was added the aryl-pyrrolidinyl-2-carboxylic acid coupling partner (Compounds 43, 45, 46 (purchased from Chem Impex International), 0.442 mmol, 1.1 equivalent), HBTU (183 mg, 0.483 mmol), HOBt (12 mg, 0.08 mmol), and dimethylsulfoxide (3 mL). N,N-Diisopropylethylamine (138 □L, 0.8 mmol) was added and the solution was stirred for 20 minutes at room temperature. At this time, methyl (S)-2-amino-3-(4′-(ethoxymethyl)-2′,6′-dimethoxy-[1,1′-biphenyl]-4-yl) propanoate (39, 0.15 g, 0.4 mmol) was added and the reaction was stirred overnight at room temperature. The reaction was partitioned between ethyl acetate and water, layers were separated and the ethyl acetate layer was dried using sodium sulfate, filtered and concentrated in vacuo. The residue was chromatographed on silica gel using ethyl acetate and hexanes as eluent. Pure fractions were pooled and concentrated in vacuo. Isolated products (see Table 4, compounds 47-49) were carried forward as-is.

TABLE 4
Intermediates

| S.M. | | Product | | LC/MS |
|---|---|---|---|---|
| # | S.M. Structure | # | Intermediate Structure | m/z |
| 43 | 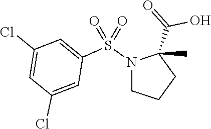 [SMILES: C[C@@]1(C(=O)O)CCCN1S(=O)(=O)c1cc(Cl)cc(Cl)c1] | 47 | 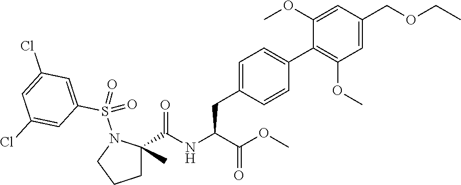 [SMILES: CCOCc1cc(OC)c(-c2ccc(C[C@H](NC(=O)[C@]3(C)CCCN3S(=O)(=O)c3cc(Cl)cc(Cl)c3)C(=O)OC)cc2)c(OC)c1] | 693.1, 695.1 |
| 45 | 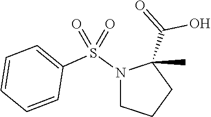 [SMILES: C[C@@]1(C(=O)O)CCCN1S(=O)(=O)c1ccccc1] | 48 | 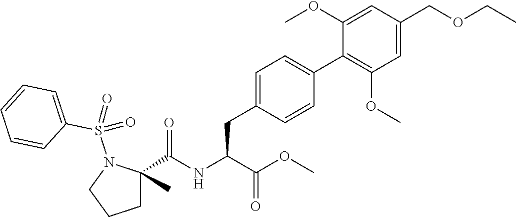 [SMILES: CCOCc1cc(OC)c(-c2ccc(C[C@H](NC(=O)[C@]3(C)CCCN3S(=O)(=O)c3ccccc3)C(=O)OC)cc2)c(OC)c1] | 625.2 |
| 46 | 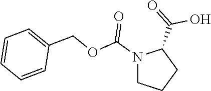 [SMILES: O=C(O)[C@@H]1CCCN1C(=O)OCc1ccccc1] | 49 | 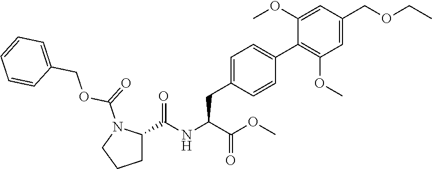 [SMILES: CCOCc1cc(OC)c(-c2ccc(C[C@H](NC(=O)[C@@H]3CCCN3C(=O)OCc3ccccc3)C(=O)OC)cc2)c(OC)c1] | 605.2 |

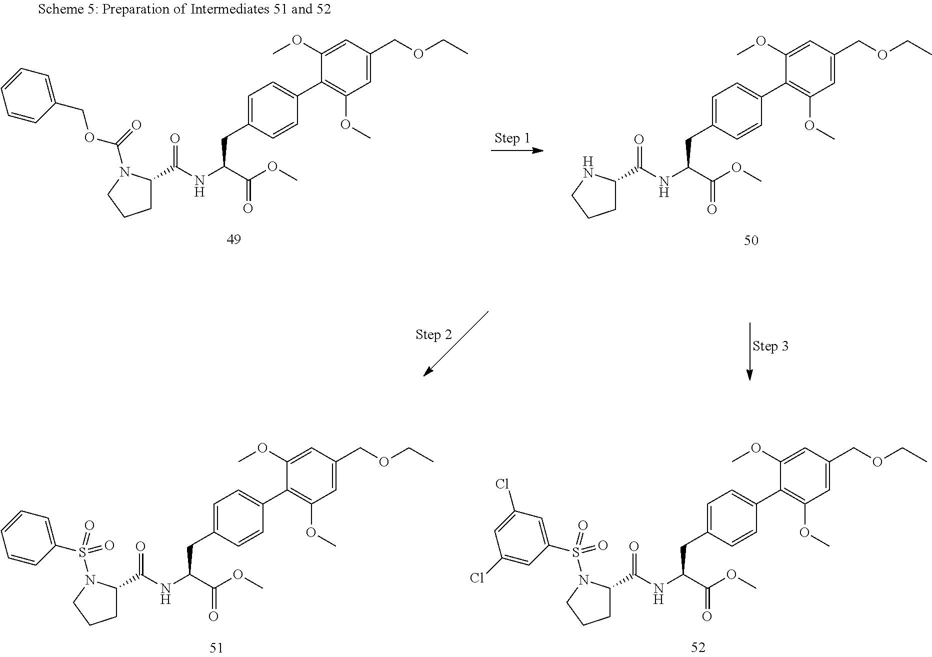 [SMILES: CCOCc1cc(OC)c(-c2ccc(C[C@H](NC(=O)[C@@H]3CCCN3C(=O)OCc3ccccc3)C(=O)OC)cc2)c(OC)c1 ; CCOCc1cc(OC)c(-c2ccc(C[C@H](NC(=O)[C@@H]3CCCN3)C(=O)OC)cc2)c(OC)c1 ; CCOCc1cc(OC)c(-c2ccc(C[C@H](NC(=O)[C@@H]3CCCN3S(=O)(=O)c3ccccc3)C(=O)OC)cc2)c(OC)c1 ; CCOCc1cc(OC)c(-c2ccc(C[C@H](NC(=O)[C@@H]3CCCN3S(=O)(=O)c3cc(Cl)cc(Cl)c3)C(=O)OC)cc2)c(OC)c1]

### Scheme 5, Step 1: Preparation of methyl (S)-3-(4′-(ethoxymethyl)-2′,6′-dimethoxy-[1,1′-biphenyl]-4-yl)-2-((S)-pyrrolidine-2-carboxamido)propanoate (50)

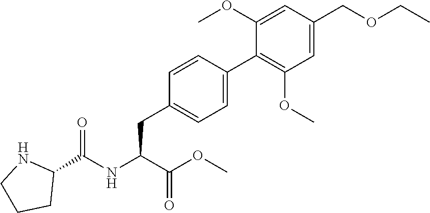 [SMILES: CCOCc1cc(OC)c(-c2ccc(C[C@H](NC(=O)[C@@H]3CCCN3)C(=O)OC)cc2)c(OC)c1]

To a round bottom flask was added benzyl (S)-2-((R)-3-((4′-(ethoxymethyl)-2′,6′-dimethoxy-[1,1′-biphenyl]-4-yl)methyl)-4-methoxy-4-oxobutanoyl)pyrrolidine-1-carboxylate (49, 0.334 g, 0.552 mmol) and methanol (5 mL). The flask was flushed with nitrogen gas, palladium on carbon (0.2 g of 10%) was added and the reaction was stirred overnight under a hydrogen atmosphere at ambient temperature and pressure. The reaction was filtered through a plug of Celite® and rinsed with additional methanol. The solvent was removed in vacuo to give product methyl (S)-3-(4′-(ethoxymethyl)-2′,6′-dimethoxy-[1,1′-biphenyl]-4-yl)-2-((S)-pyrrolidine-2-carboxamido)propanoate (50, 0.237 g, 91% yield) as an oil. LC-MS: t_{R}=2.07 min; m/z=471.2.

### Scheme 5, Step 2: Preparation of methyl (S)-3-(4′-(ethoxymethyl)-2′,6′-dimethoxy-[1,1′-biphenyl]-4-yl)-2-((S)-1-(phenylsulfonyl)pyrrolidine-2-carboxamido)propanoate (51)

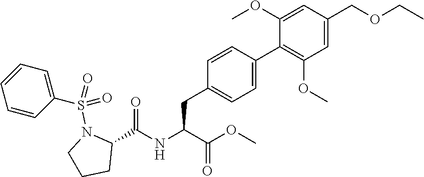 [SMILES: CCOCc1cc(OC)c(-c2ccc(C[C@H](NC(=O)[C@@H]3CCCN3S(=O)(=O)c3ccccc3)C(=O)OC)cc2)c(OC)c1]

To a round bottom flask was added methyl (S)-3-(4′-(ethoxymethyl)-2′,6′-dimethoxy-[1,1′-biphenyl]-4-yl)-2-((S)-pyrrolidine-2-carboxamido)propanoate (compound 50, 0.118 g, 0.251 mmol) and dichloromethane (2 mL). The solution was cooled to 0° C. and benzenesulfonyl chloride (66 mg, 0.376 mmol) and diisopropylethylamine (215 □L, 1.25 mmol) were added. After stirring for 1 hour at room temperature, the reaction was taken up in ethyl acetate and washed with hydrochloric acid (10 mL, 1N), water, and then brine. The ethyl acetate layer was dried over sodium sulfate, filtered and concentrated in vacuo. The residue was purified on C18 using acetonitrile and water (both with 0.1% formic acid as modifier). Pure fractions were pooled and concentrated in vacuo. Product methyl (S)-3-(4′-(ethoxymethyl)-2′,6′-dimethoxy-[1,1′-biphenyl]-4-yl)-2-((S)-1-(phenylsulfonyl)pyrrolidine-2-carboxamido) propanoate (51, 0.112 g, 73% yield) was isolated as an oil. LC-MS: t_{R}=2.66 min; m/z=611.2.

### Scheme 5, Step 3: Preparation of methyl (S)-2-((S)-1-((3,5-dichlorophenyl)sulfonyl)-pyrrolidine-2-carboxamido)-3-(4′-(ethoxymethyl)-2′,6′-dimethoxy-[1,1′-biphenyl]-4-yl)propanoate (52)

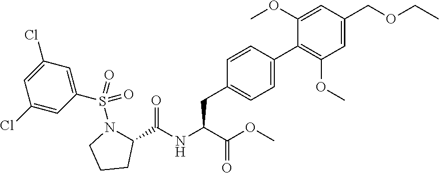 [SMILES: CCOCc1cc(OC)c(-c2ccc(C[C@H](NC(=O)[C@@H]3CCCN3S(=O)(=O)c3cc(Cl)cc(Cl)c3)C(=O)OC)cc2)c(OC)c1]

To a round bottom flask was added methyl (S)-3-(4′-(ethoxymethyl)-2′,6′-dimethoxy-[1,1′-biphenyl]-4-yl)-2-((S)-pyrrolidine-2-carboxamido)propanoate (compound 50, 0.118 g, 0.251 mmol) and dichloromethane (2 mL). The solution was cooled to 0° C. and 3,5-dichlorobenzenesulfonyl chloride (66 mg, 0.376 mmol) and diisopropylethylamine (215 μL, 1.25 mmol) were added and the reaction was stirred overnight at room temperature. The reaction was taken up in ethyl acetate and washed with hydrochloric acid (10 mL, 1N), water, and then brine. The ethyl acetate layer was dried over sodium sulfate, filtered and concentrated in vacuo. The residue was purified on C18 using acetonitrile and water (both with 0.1% formic acid as modifier). Pure fractions were pooled and concentrated in vacuo. Product methyl (S)-2-((S)-1-((3,5-dichlorophenyl)sulfonyl)pyrrolidine-2-carboxamido)-3-(4′-(ethoxymethyl)-2′,6′-dimethoxy-[1,1′-biphenyl]-4-yl)propanoate (52, 0.121 g, 71% yield) was isolated as an oil. LC-MS: t_{R}=2.92 min; m/z=679.1, 681.1.

### Scheme 2, Step 6: General Procedure D—Hydrolysis (Examples 1608-1611)

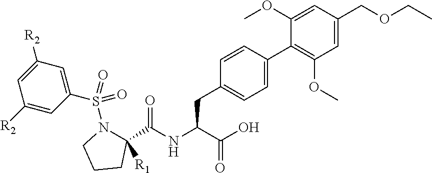

To a flask was added the appropriate ester intermediate (Intermediates 47, 48, 51, 52), acetonitrile (1 mL), water (1 mL) and lithium hydroxide monohydrate (5 equivalents). The solution was stirred at room temperature for 2 hours. Acetonitrile was removed in vacuo and the reaction was acidified using hydrochloric acid (1N). Water was then removed in vacuo and the residue was purified on C18 using acetonitrile and water (both with 0.1% formic acid as modifier). Pure fractions were pooled and concentrated in vacuo and the resulting material was lyophilized from acetonitrile and water (1:4) overnight. Isolated products are shown in Table 5 (Examples 1608-1611).

TABLE 5
Example Compounds and Characterization

| Ex. | | | | | LC/MS |
|---|---|---|---|---|---|
| No. | IUPAC Name | Structure | Yield | ¹H NMR δ | m/z |
| Cmpd 1608 | (S)-2-((S)-1-((3,5- dichlorophenyl)sulfonyl)-2- methylpyrrolidine-2-carboxamido)- 3-(4′-(ethoxymethyl)-2′,6′- dimethoxy-[1,1′-biphenyl]-4- yl)propanoic acid | 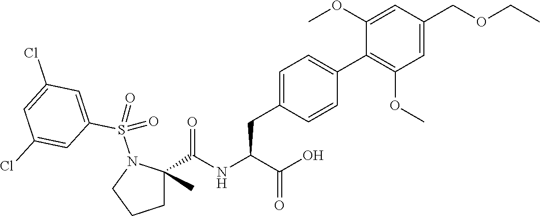 [SMILES: CCOCc1cc(OC)c(-c2ccc(C[C@H](NC(=O)[C@]3(C)CCCN3S(=O)(=O)c3cc(Cl)cc(Cl)c3)C(=O)O)cc2)c(OC)c1] | 66% | ¹H NMR (400 MHz, DMSO- d6) δ ppm 12.86 (br. s., 1 H) 7.97 (t, J = 1.96 Hz, 1 H) 7.81 (d, J = 1.71 Hz, 2 H) 7.70 (d, J = 7.82 Hz, 1 H) 7.20 (d, J = 8.31 Hz, 2 H) 7.09 (d, J = 8.31 Hz, 2 H) 6.66 (s, 2 H) 4.50 (m, J = 4.90 Hz, 1 H) 4.46 (s, 2 H) 3.62 (s, 6 H) 3.51 (q, J = 7.09 Hz, 2 H) 3.37 (m, J = 7.10 Hz, 2 H) 3.12- 3.22 (m, 1 H) 2.96-3.08 (m, 1 H) 1.92 (m, J = 7.30 Hz, 1 H) 1.67-1.80 (m, 2 H) 1.61-1.69 | 679.1, 681.1 |
| | | | | (m, 1 H) 1.49 (s, 3 H) 1.18 (t, | |
| | | | | J = 6.97 Hz, 3 H). | |
| Cmpd 1609 | (S)-3-(4′-(ethoxymethyl)-2′,6′- dimethoxy-[1,1′-biphenyl]-4-yl)-2- ((S)-2-methyl-1- (phenylsulfonyl)pyrrolidine-2- carboxamido)propanoic acid | 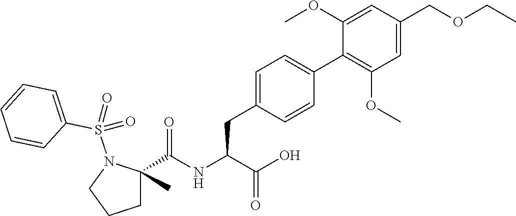 [SMILES: CCOCc1cc(OC)c(-c2ccc(C[C@H](NC(=O)[C@]3(C)CCCN3S(=O)(=O)c3ccccc3)C(=O)O)cc2)c(OC)c1] | 73% | ¹H NMR (400 MHz, DMSO-d6) δ ppm 12.90 (br. s., 1 H) 7.79-7.88 (m, 2 H) 7.61-7.70 (m, 1 H) 7.50- 7.60 (m, 3 H) 7.19 (d, J = 8.07 Hz, 2 H) 7.09 (d, J = 8.07 Hz, 2 H) 6.66 (s, 2 H) 4.52 (m, J = 4.90 Hz, 1 H) 4.46 (s, 2 H) 3.62 (s, 6 H) 3.52 (q, J = 7.01 Hz, 2 H) 3.37 (m, J = 5.10 Hz, 1 H) 3.23-3.30 (m, 1 H) 3.19 (dd, J = 13.94, 4.89 Hz, 1 H) 3.02 (dd, J = 1.00 Hz, 1 H) 1.86-1.97 (m, 1 H) 1.49-1.71 (m, 3 H) 1.43 (s, 3 H) 1.18 (t, J = 6.97 Hz, 3 H). | 611.2 |
| Cmpd 1610 | (S)-3-(4′-(ethoxymethyl)-2′,6′- dimethoxy-[1,1′-biphenyl]-4-yl)-2- ((S)-1-(phenylsulfonyl)pyrrolidine-2- carboxamido)propanoic acid | 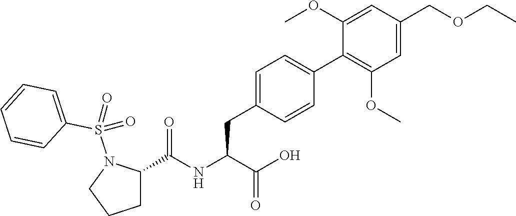 [SMILES: CCOCc1cc(OC)c(-c2ccc(C[C@H](NC(=O)[C@@H]3CCCN3S(=O)(=O)c3ccccc3)C(=O)O)cc2)c(OC)c1] | 93% | ¹H NMR (400 MHz, DMSO- d6) δ ppm 12.90 (br. s., 1 H) 8.17 (d, J = 8.07 Hz, 1 H) 7.82 (d, J = 7.58 Hz, 2 H) 7.65-7.74 (m, 1 H) 7.55-7.64 (m, 2H) 7.22 (d, J = 8.07 Hz, 2 H) 7.10 (d, J = 7.82 Hz, 2 H) 6.66 (s, 2 H) 4.47-4.55 (m, 1 H) 4.45 (s, 2 H) 4.14 (m, J = 7.60 Hz, 1 H) 3.61 (s, 6 H) 3.51 (q, J = 7.09 Hz, 2 H) 3.31-3.38 (m, 1H) 3.07-3.19 (m, 2 H) 2.94-3.05 (m, 1 H) 1.46-1.65 (m, 3 H) | 597.2 |
| | | | | 1.40 (m, J = 6.10 Hz, 1 H) 1.17 | |
| | | | | (t, J = 7.09 Hz, 3 H). | |
| Cmpd 1611 | (S)-2-((S)-1-((3,5- dichlorophenyl)sulfonyl)pyrrolidine- 2-carboxamido)-3-(4′- (ethoxymethyl)-2′,6′-dimethoxy- [1,1′-biphenyl]-4-yl)propanoic acid | 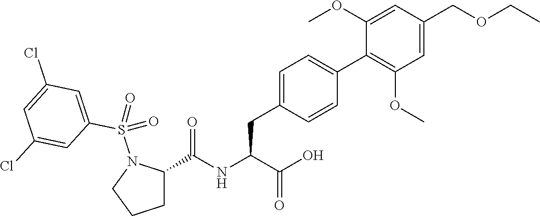 [SMILES: CCOCc1cc(OC)c(-c2ccc(C[C@H](NC(=O)[C@@H]3CCCN3S(=O)(=O)c3cc(Cl)cc(Cl)c3)C(=O)O)cc2)c(OC)c1] | 84% | ¹H NMR (400 MHz, DMSO- d6) δ ppm 12.89 (s, 1 H) 8.31 (d, J = 8.07 Hz, 1 H) 8.02 (s, 1 H) 7.85 (d, J = 1.47 Hz, 2 H) 7.23 (d, J = 7.82 Hz, 2 H) 7.10 (d, J = 7.83 Hz, 2 H) 6.66 (s, 2 H) 4.48-4.57 (m, 1 H) 4.46 (s, 2 H) 4.26-4.34 (m, 1 H) 3.62 (s, 6 H) 3.51 (q, J = 6.93 Hz, 2 H) 3.35-3.41 (m, 1 H) 3.23 (m, J = 9.30 Hz, 1 H) 3.06-3.15 (m, 1 H) 2.94-3.05 (m, 1 H) 1.61- 1.79 (m, 3 H) 1.54 (m, J = 4.90 | 665.1, 667.1 |
| | | | | Hz, 1 H) 1.18 (t, J = 6.97 Hz, 3 | |
| | | | | H). | |

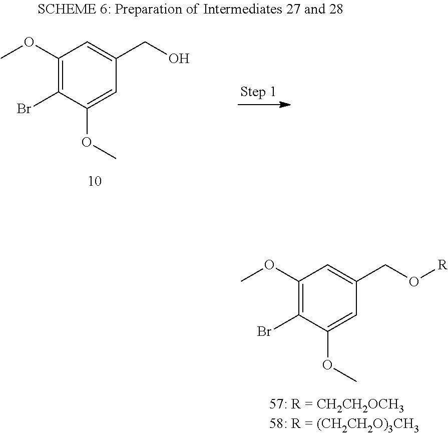

### Scheme 6, Step 1: Preparation of 2-Bromo-1,3-dimethoxy-5-((2-methoxyethoxy) methy)benzene (57)

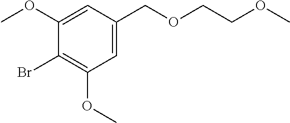

To a round bottom flask was added (4-bromo-3,5-dimethoxyphenyl)methanol (compound 10, 1.95 g, 7.89 mmol) and N,N-dimethylformamide (20 mL). The solution was cooled to 0° C. and sodium hydride (0.393 g of 60% (w/w) in mineral oil, 10.3 mmol) was added and the reaction was stirred for 1 hour at room temperature. 1-Bromo-2-methoxyethane (0.89 mL, 9.47 mmol) was added and the reaction was stirred overnight at room temperature. The reaction was quenched with methanol and concentrated in vacuo. The residue was taken up in ethyl acetate and washed using water, then brine. The organic layer was dried using sodium sulfate, filtered and concentrated in vacuo. The resulting oil was purified on silica gel using ethyl acetate and hexanes as eluent. Product 2-Bromo-1,3-dimethoxy-5-((2-methoxyethoxy)methy)benzene (57, 2.22 g, 92% yield) was isolated as a pale yellow oil. LC-MS: t_{R}=2.11 min; m/z+23=327.0, 329.0. ¹H NMR (400 MHz, DMSO-d6) δ ppm 6.70 (s, 2H) 4.48 (s, 2H) 3.82 (s, 6H) 3.53-3.62 (m, 2H) 3.45-3.52 (m, 2H) 3.26 (s, 3H).

### Preparation of 1-(4-Bromo-3,5-dimethoxyphenyl-2,5,8,11-tetraoxadodecane (58)

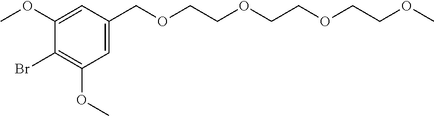

To a round bottom flask was added (4-bromo-3,5-dimethoxyphenyl)methanol (compound 10, 1.9 g, 7.69 mmol) and N,N-dimethylformamide (20 mL). The solution was cooled to 0° C. and sodium hydride (0.383 g of 60% (w/w) in mineral oil, 10 mmol) was added and the reaction was stirred for 1 hour at room temperature. 2-[2-(2-Methoxyethoxy)ethoxy]ethyl 4-methylbenzenesulfonate (2.8 mL, 9.23 mmol) was added and the reaction was stirred for six hours at room temperature. The reaction was quenched with water and concentrated in vacuo. The residue was taken up in ethyl acetate and washed using water, then brine. The organic layer was dried using sodium sulfate, filtered and concentrated in vacuo. The resulting oil was purified on silica gel using ethyl acetate and hexanes as eluent. Product 1-(4-Bromo-3,5-dimethoxyphenyl-2,5,8,11-tetraoxadodecane (58, 3.02 g, quant. yield) was isolated as a clear oil. LC-MS: t_{R}=2.08 min; m/z+23=415.0, 417.0. ¹H NMR (400 MHz, DMSO-d6) δ ppm 6.70 (s, 2H) 4.48 (s, 2H) 3.82 (s, 6H) 3.57 (s, 4H) 3.47-3.55 (m, 6H) 3.39-3.45 (m, 2H) 3.22 (s, 3H).

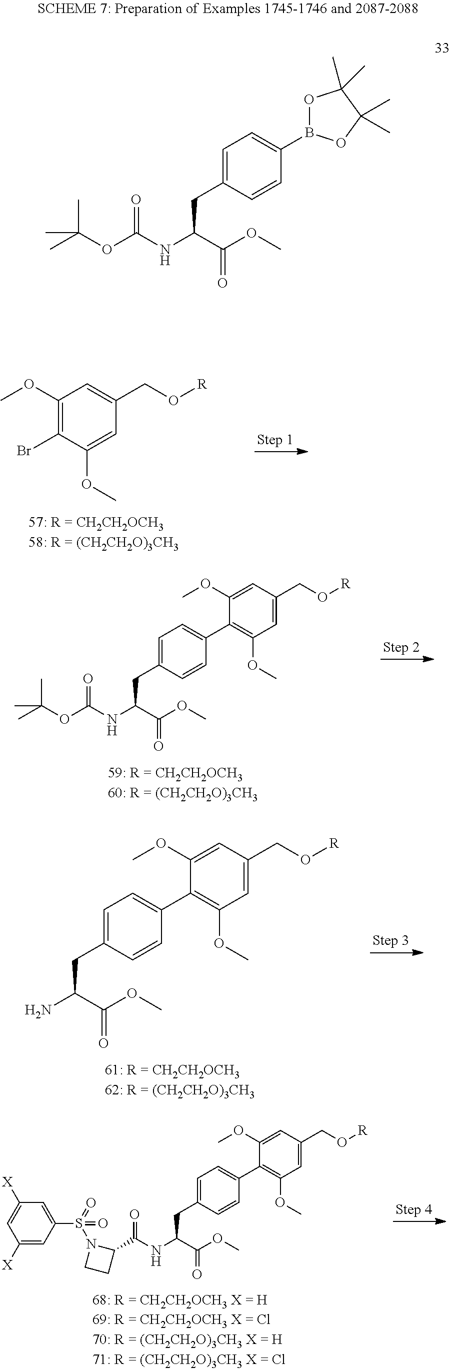

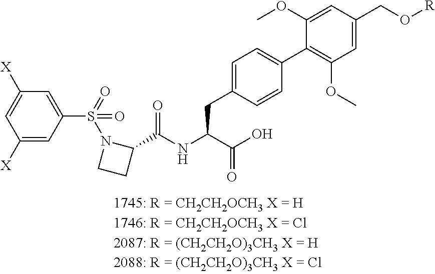

### Scheme 7, Step 1: Preparation of methyl (S)-2-((tert-butoxycarbonyl)amino)-3-(2′,6′-dimethoxy-4′-((2-methoxyethoxy)methyl)-[1,1′-biphenyl]-4-yl)propanoate (59)

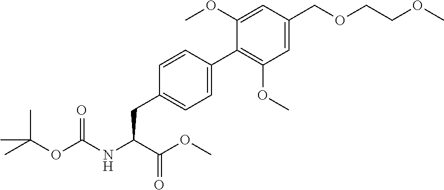 [SMILES: COCCOCc1cc(OC)c(-c2ccc(C[C@H](NC(=O)OC(C)(C)C)C(=O)OC)cc2)c(OC)c1]

To a microwave vial was added methyl (S)-2-((tert-butoxycarbonyl)amino)-3-(4-(4,4,5,5-tetramethyl-1,3,2-dioxaborolan-2-yl)phenyl)propanoate (33, 599 mg, 1.48 mmol), 2-Bromo-1,3-dimethoxy-5-((2-methoxyethoxy)methy)benzene (57, 631 mg, 2.07 mmol), tetrakis (triphenylphosphane)palladium(0) (85 mg, 0.074 mmol), cesium acetate (851 mg, 4.43 mmol), 1,4-dioxane (5 mL) and water (1 mL). The vial was crimped shut and sparged with nitrogen for 10 minutes and then heated for 48 hours at 120° C. The reaction was partitioned between ethyl acetate and water, layers were separated and the ethyl acetate layer was dried using sodium sulfate, filtered and concentrated in vacuo. The residue was chromatographed on silica gel using ethyl acetate and hexanes as eluent. Pure fractions were pooled and concentrated in vacuo to give product methyl (S)-2-((tert-butoxycarbonyl)amino)-3-(2′,6′-dimethoxy-4′-((2-methoxyethoxy) methyl)-[1,1′-biphenyl]-4-yl)propanoate (59, 262 mg, 35% yield) as an oil. LC-MS: t_{R}=2.59 min; m/z+23=526.1.

### Scheme 7, Step 1: Preparation of methyl (S)-3-(4′-(2,5,8,11-tetraoxadodecyl)-2′,6′-dimethoxy-[1,1′-biphenyl]-4-yl)-2-((tert-butoxycarbonyl)amino)propanoate (60)

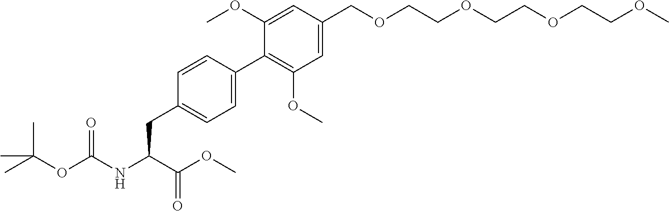 [SMILES: COCCOCCOCCOCc1cc(OC)c(-c2ccc(C[C@H](NC(=O)OC(C)(C)C)C(=O)OC)cc2)c(OC)c1]

To a microwave vial was added methyl (S)-2-((tert-butoxycarbonyl)amino)-3-(4-(4,4,5,5-tetramethyl-1,3,2-dioxaborolan-2-yl)phenyl)propanoate (33, 720 mg, 1.78 mmol), 1-(4-Bromo-3,5-dimethoxyphenyl-2,5,8,11-tetraoxadodecane (58, 759 mg, 1.93 mmol), tetrakis (triphenylphosphane)palladium(0) (103 mg, 0.089 mmol), cesium acetate (1.02 g, 5.33 mmol), 1,4-dioxane (5 mL) and water (1 mL). The vial was crimped shut and sparged with nitrogen for 10 minutes and then heated for 24 hours at 120° C. The reaction was partitioned between ethyl acetate and water, layers were separated and the ethyl acetate layer was dried using sodium sulfate, filtered and concentrated in vacuo. The residue was chromatographed on silica gel using ethyl acetate and hexanes as eluent. Pure fractions were pooled and concentrated in vacuo to give product methyl (S)-3-(4′-(2,5,8,11-tetraoxadodecyl)-2′,6′-dimethoxy-[1,1′-biphenyl]-4-yl)-2-((tert-butoxycarbonyl)amino)propanoate (60, 352 mg, 33.5% yield) as an oil. LC-MS: t_{R}=2.56 min; m/z+23=614.3.

### Scheme 7, Step 2: Preparation of methyl (S)-2-amino-3-(2′,6′-dimethoxy-4′-((2-methoxyethoxy)methyl)-[1,1′-biphenyl]-4-yl)propanoate (61)

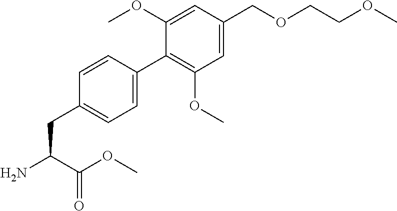 [SMILES: COCCOCc1cc(OC)c(-c2ccc(C[C@H](N)C(=O)OC)cc2)c(OC)c1]

To a round bottom flask was added methyl (S)-2-((tert-butoxycarbonyl)amino)-3-(2′,6′-dimethoxy-4′-((2-methoxyethoxy)methyl)-[1,1′-biphenyl]-4-yl)propanoate (59, 262 mg, 0.520 mmol) and trifluoroacetic acid in dichloromethane (10 mL, 20% v/v) and the reaction was stirred for 30 minutes at room temperature. The reaction was concentrated in vacuo and then partitioned between ethyl acetate and saturated sodium bicarbonate, layers were separated and the ethyl acetate layer was dried using sodium sulfate, filtered and concentrated in vacuo. The residue was chromatographed on silica gel using methanol and dichloromethane as eluent. Pure fractions were pooled and concentrated in vacuo to give product methyl (S)-2-amino-3-(2′,6′-dimethoxy-4′-((2-methoxyethoxy)methyl)-[1,1′-biphenyl]-4-yl)propanoate (61, 210 mg, 100% yield) as an oil. LC-MS: t_{R}=1.82 min; m/z=404.2.

### Scheme 7, Step 2: Preparation of methyl (S)-3-(4′-(2,5,8,11-tetraoxadodecyl)-2′,6′-dimethoxy-[1,1′-biphenyl]-4-yl)-2-aminopropanoate (62)

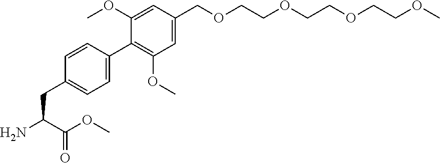 [SMILES: COCCOCCOCCOCc1cc(OC)c(-c2ccc(C[C@H](N)C(=O)OC)cc2)c(OC)c1]

To a round bottom flask was added methyl (S)-3-(4′-(2,5,8,11-tetraoxadodecyl)-2′,6′-dimethoxy-[1,1′-biphenyl]-4-yl)-2-((tert-butoxycarbonyl)amino)propanoate (60, 352 mg, 0.595 mmol) and trifluoroacetic acid in dichloromethane (10 mL, 20% v/v) and the reaction was stirred for 30 minutes at room temperature. The reaction was concentrated in vacuo and then partitioned between ethyl acetate and saturated sodium bicarbonate, layers were separated and the ethyl acetate layer was dried using sodium sulfate, filtered and concentrated in vacuo. The residue was chromatographed on silica gel using methanol and dichloromethane as eluent. Pure fractions were pooled and concentrated in vacuo to give product methyl (S)-3-(4′-(2,5,8,11-tetraoxadodecyl)-2′,6′-dimethoxy-[1,1′-biphenyl]-4-yl)-2-aminopropanoate (62, 292 mg, 99.8% yield) as an oil. LC-MS: t_{R}=1.81 min; m/z=492.2.

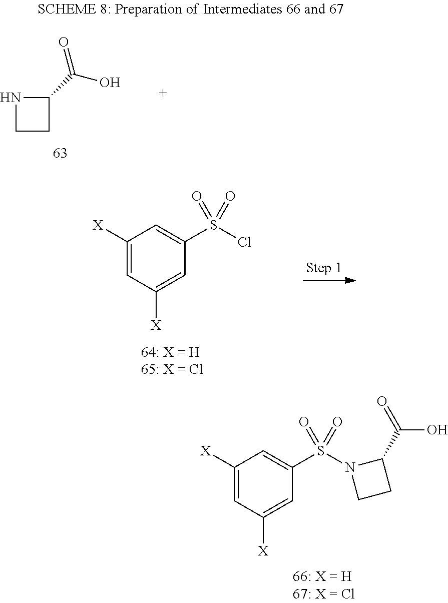

### Scheme 8, Step 1: Preparation of (S)-1-(phenylsulfonyl)azetidine-2-carboxylic Acid (66)

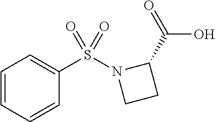 [SMILES: O=C(O)[C@@H]1CCN1S(=O)(=O)c1ccccc1]

To a round bottom flask was added (S)-azetidine-2-carboxylic acid (63, purchased from Chem Impex International, 500 mg, 4.95 mmol) and sodium hydroxide (6.9 mL of 1N; 6.92 mmol). The reaction was cooled to 0° C. and benzenesulfonyl chloride (64, 0.96 g, 5.44 mmol) was added followed by N,N-diisopropylethylamine (0.97 mL, 5.69 mmol) and acetone (7 mL) and the reaction was stirred overnight at room temperature. The acetone was evaporated and the aqueous layer extracted with diethyl ether (3×50 mL). The aqueous layer was adjusted to pH=1 using conc. HCl and then extracted with ethyl acetate (3×75 mL). The ethyl acetate layers were pooled, dried using sodium sulfate, filtered and concentrated in vacuo to give product (S)-1-(phenylsulfonyl)azetidine-2-carboxylic acid (66, 1.14 g, 95% yield) as a white solid. LC-MS: t_{R}=1.46 min; m/z=241.9. ¹H NMR (400 MHz, DMSO-d6) δ ppm 12.98 (s, 1H) 7.80-7.89 (m, 2H) 7.73-7.80 (m, 1H) 7.64-7.72 (m, 2H) 4.34 (t, J=8.68 Hz, 1H) 3.69 (td, J=7.64, 5.75 Hz, 1H) 3.60 (q, J=8.23 Hz, 1H) 2.15-2.26 (m, 2H).

### Scheme 8, Step 1: Preparation of (S)-1-((3,5-dichlorophenyl)sulfonyl)azetidine-2-carboxylic Acid (67)

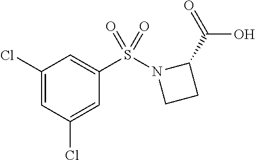 [SMILES: O=C(O)[C@@H]1CCN1S(=O)(=O)c1cc(Cl)cc(Cl)c1]

To a round bottom flask was added (S)-azetidine-2-carboxylic acid (63, 511 mg, 5.05 mmol) and sodium hydroxide (7.0 mL of 1N; 7.08 mmol). The reaction was cooled to 0° C. and 3,5-dichlorobenzenesulfonyl chloride (65, 1.36 g, 5.56 mmol) was added followed by N,N-diisopropylethylamine (1.0 mL, 5.81 mmol) and acetone (7 mL) and the reaction was stirred overnight at room temperature. The acetone was evaporated and the aqueous layer extracted with diethyl ether (3×50 mL). The aqueous layer was adjusted to pH=1 using conc. HCl and then extracted with ethyl acetate (3×75 mL). The ethyl acetate layers were pooled, dried using sodium sulfate, filtered and concentrated in vacuo to give product (S)-1-((3,5-dichlorophenyl)sulfonyl)azetidine-2-carboxylic acid (67, 1.6 g, 100% yield) as a white solid. LC-MS: t_{R}=2.06 min; m/z=309.8, 311.9. ¹H NMR (400 MHz, DMSO-d6) δ ppm 13.08 (br. s., 1H) 8.06 (t, J=1.83 Hz, 1H) 7.85 (d, J=1.96 Hz, 2H) 4.63 (dd, J=9.54, 7.58 Hz, 1H) 3.67-3.88 (m, 2H) 2.29-2.42 (m, 1H) 2.13-2.28 (m, 1H).

### Scheme 7, Step 3: General Procedure E—Amide Coupling (Intermediates 68-71)

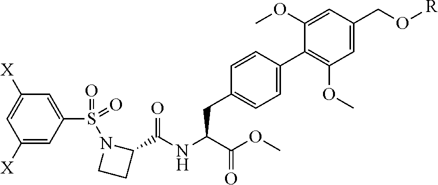

To a round bottom flask was added the aryl-azetidine-2-carboxylic acid coupling partner (Compounds 66 or 67, 1.1 equivalent), HATU (2.2 equivalents), HOBt (0.2 equivalent), and dimethylsulfoxide (1 mL). N,N-Diisopropylethylamine (3 equivalents) was added and the solution was stirred for 20 minutes at room temperature. At this time, the appropriate amine (61 or 62, 1 equivalent) was added and the reaction was stirred overnight at room temperature. The reaction was partitioned between ethyl acetate and water, layers were separated and the ethyl acetate layer was dried using sodium sulfate, filtered and concentrated in vacuo. The residue was chromatographed on silica gel using ethyl acetate and hexanes as eluent. Pure fractions were pooled and concentrated in vacuo. Isolated products (see Table 6, compounds 68-71) were carried forward as-is.

TABLE 6
Intermediates

| S.M. | | Product | | LC/MS |
|---|---|---|---|---|
| #'s | Starting Acid | # | Intermediate Structure | m/z |
| 61 66 | 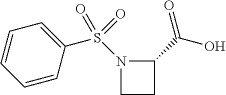 [SMILES: O=C(O)[C@@H]1CCN1S(=O)(=O)c1ccccc1] | 68 | 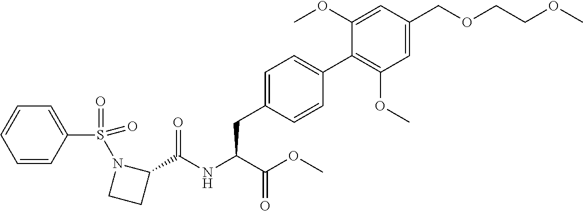 [SMILES: COCCOCc1cc(OC)c(-c2ccc(C[C@H](NC(=O)[C@@H]3CCN3S(=O)(=O)c3ccccc3)C(=O)OC)cc2)c(OC)c1] | 627.2 |
| 61 67 | 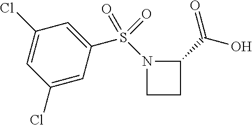 [SMILES: O=C(O)[C@@H]1CCN1S(=O)(=O)c1cc(Cl)cc(Cl)c1] | 69 | 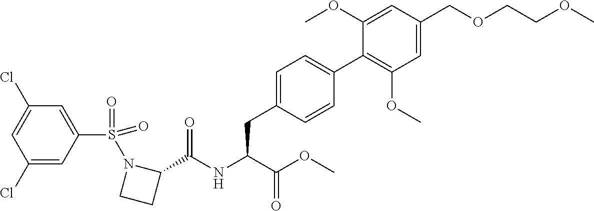 [SMILES: COCCOCc1cc(OC)c(-c2ccc(C[C@H](NC(=O)[C@@H]3CCN3S(=O)(=O)c3cc(Cl)cc(Cl)c3)C(=O)OC)cc2)c(OC)c1] | 695.1 697.1 |
| 62 66 | 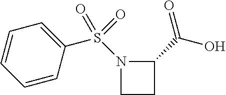 [SMILES: O=C(O)[C@@H]1CCN1S(=O)(=O)c1ccccc1] | 70 | 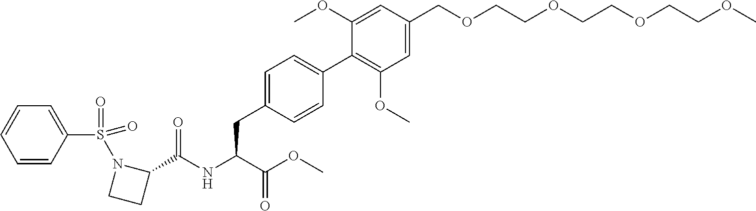 [SMILES: COCCOCCOCCOCc1cc(OC)c(-c2ccc(C[C@H](NC(=O)[C@@H]3CCN3S(=O)(=O)c3ccccc3)C(=O)OC)cc2)c(OC)c1] | 737.2 (M + 23) |
| 62 67 | 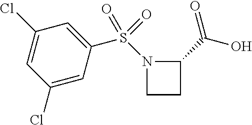 [SMILES: O=C(O)[C@@H]1CCN1S(=O)(=O)c1cc(Cl)cc(Cl)c1] | 71 | 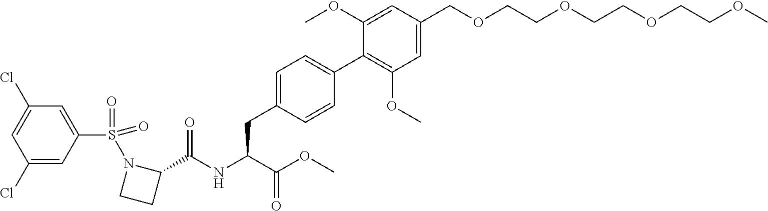 [SMILES: COCCOCCOCCOCc1cc(OC)c(-c2ccc(C[C@H](NC(=O)[C@@H]3CCN3S(=O)(=O)c3cc(Cl)cc(Cl)c3)C(=O)OC)cc2)c(OC)c1] | 805.1 807.1 (M + 23) |

### Scheme 7, Step 4: Hydrolysis (Examples 142-145)

Intermediates 68-71 were hydrolyzed using General Procedure D (Scheme 2, Step 6) to give examples 1745-1746 and 2087-2088 (see Table 7).

TABLE 7
Example Compounds and Characterization

| Ex. | | | | | LC/MS |
|---|---|---|---|---|---|
| No. | IUPAC Name | Structure | Yield | ¹H NMR δ | m/z |
| Cmpd 1745 | (S)-3-(2′,6′-dimethoxy-4′-((2- methoxyethoxy)methyl)-[1,1′- biphenyl]-4-yl)-2-((S)-1- (phenylsulfonyl)azetidine-2- carboxamido)propanoic acid | 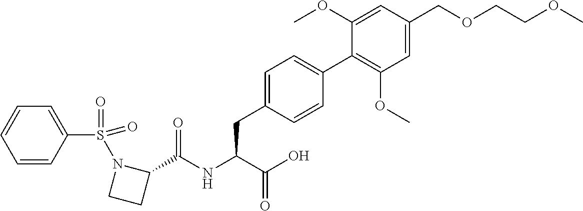 [SMILES: COCCOCc1cc(OC)c(-c2ccc(C[C@H](NC(=O)[C@@H]3CCN3S(=O)(=O)c3ccccc3)C(=O)O)cc2)c(OC)c1] | 62% | ¹H NMR (400 MHz, DMSO- d6) δ ppm 12.97 (br. s, 1 H) 8.18 (d, J = 8.07 Hz, 1 H) 7.81- 7.87 (m, 2 H) 7.74-7.80 (m, 1 H) 7.64-7.70 (m, 2 H) 7.23 (d, J = 8.07 Hz, 2 H) 7.12 (d, J = 8.31 Hz, 2 H) 6.68 (s, 2 H) 4.53 (m, J = 5.40 Hz, 1 H) 4.50 (s, 2 H) 4.30 (dd, J = 9.29, 7.34 Hz, 1 H) 3.67 (m, J = 4.40 Hz, 1 H) 3.62 (s, 6 H) 3.57-3.61 (m, 2 H) 3.55 (d, J = 9.05 Hz, 1 H) 3.48- 3.53 (m, 2 H) 3.27 (s, 3 H) 3.13 | 613.2 |
| | | | | (m, J = 5.10 Hz, 1 H) 3.03 (m, | |
| | | | | J = 8.30 Hz, 1 H) 2.00-2.11 (m, | |
| | | | | 1 H) 1.86-1.98 (m, 1H) | |
| Cmpd 1746 | (S)-2-((S)-1-((3,5- dichlorophenyl)sulfonyl) azetidine-2-carboxamido)-3- (2′,6′-dimethoxy-4′-((2- methoxyethoxy)methyl)-[1,1′- biphenyl]-4-yl)propanoic acid | 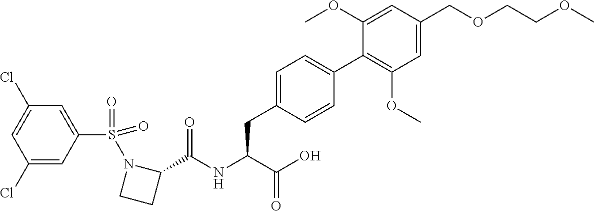 [SMILES: COCCOCc1cc(OC)c(-c2ccc(C[C@H](NC(=O)[C@@H]3CCN3S(=O)(=O)c3cc(Cl)cc(Cl)c3)C(=O)O)cc2)c(OC)c1] | 61% | ¹H NMR (400 MHz, DMSO- d6) δ ppm 12.97 (br. s, 1 H) 8.32 (d, J = 7.82 Hz, 1 H) 8.07 (t, J = 1.83 Hz, 1 H) 7.86 (d, J = 1.96 Hz, 2 H) 7.23 (d, J = 8.31 Hz, 2 H) 7.12 (d, J = 8.31 Hz, 2 H) 6.68 (s, 2 H) 4.54 (dd, J = 9.17, 7.21 Hz, 2 H) 4.50 (s, 2 H) 3.67- 3.77 (m, 2 H) 3.62 (s, 6 H) 3.56-3.61 (m, 2 H) 3.49-3.54 (m, 2 H) 3.28 (s, 3 H) 3.10 (m, J = 5.60 Hz, 1 H) 3.03 (m, J = 8.10 Hz, 1 H) 2.14-2.25 (m, | 703.1 705.1 (M + 23) |
| | | | | 1 H) 1.93-2.06 (m, 1 H) | |
| Cmpd 2087 | (S)-3-(4′-(2,5,8,11- tetraoxadodecyl)-2′,6′- dimethoxy-[1,1′-biphenyl]-4- yl)-2-((S)-1- (phenylsulfonyl)azetidine-2- carboxamido)propanoic acid | 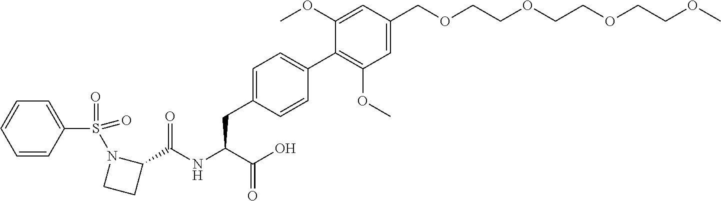 [SMILES: COCCOCCOCCOCc1cc(OC)c(-c2ccc(C[C@H](NC(=O)[C@@H]3CCN3S(=O)(=O)c3ccccc3)C(=O)O)cc2)c(OC)c1] | 85% | ¹H NMR (400 MHz, DMSO- d6) δ ppm 12.96 (br. s, 1 H) 8.18 (d, J = 8.07 Hz, 1 H) 7.81- 7.87 (m, 2 H) 7.74-7.81 (m, 1 H) 7.64-7.71 (m, 2 H) 7.24 (d, J = 8.07 Hz, 2 H) 7.12 (d, J = 8.07 Hz, 2 H) 6.68 (s, 2 H) 4.52- 4.57 (m, 1 H) 4.51 (s, 2 H) 4.30 (dd, J = 9.29, 7.34 Hz, 1 H) 3.67 (m, J = 4.60 Hz, 1 H) 3.62 (s, 6 H) 3.59 (s, 4 H) 3.48-3.57 (m, 7 H) 3.38-3.44 (m, 2 H) 3.22 | 723.2 (M + 23) |
| | | | | (s, 3 H) 3.14 (dd, J = 1.00 Hz, 1 | |
| | | | | H) 3.03 (dd, J = 7.80 Hz, 1 H) | |
| | | | | 1.99-2.12 (m, 1 H) 1.86-1.98 | |
| | | | | (m, 1 H) | |
| Cmpd 2088 | (S)-3-(4′-(2,5,8,11- tetraoxadodecyl)-2′,6′- dimethoxy-[1,1′-biphenyl]-4- yl)-2-((S)-1-((3,5- dichlorophenyl)sulfonyl) azetidine-2-carboxamido) propanoic acid | 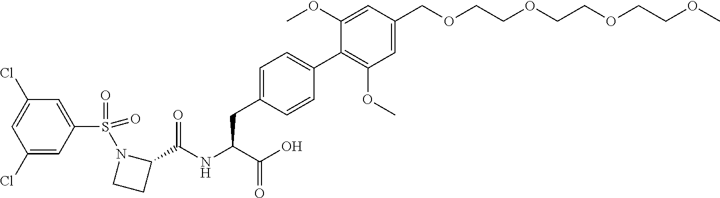 [SMILES: COCCOCCOCCOCc1cc(OC)c(-c2ccc(C[C@H](NC(=O)[C@@H]3CCN3S(=O)(=O)c3cc(Cl)cc(Cl)c3)C(=O)O)cc2)c(OC)c1] | 73% | ¹H NMR (400 MHz, DMSO- d6) δ ppm 12.96 (br. s, 1 H) 8.32 (d, J = 7.82 Hz, 1 H) 8.07 (t, J = 1.83 Hz, 1 H) 7.86 (d, J = 1.96 Hz, 2 H) 7.23 (d, J = 8.31 Hz, 2 H) 7.12 (d, J = 8.07 Hz, 2 H) 6.68 (s, 2 H) 4.52-4.58 (m, 2 H) 4.51 (s, 2 H) 3.65-3.80 (m, 2 H) 3.62 (s, 6 H) 3.59 (s, 4 H) 3.47-3.57 (m, 6 H) 3.39-3.44 (m, 2 H) 3.22 (s, 3 H) 3.11 (dd, J = 5.10 Hz, 1 H) 3.03 (dd, | 791.1 793.1 (M + 23) |
| | | | | J = 8.10 Hz, 1 H) 2.12-2.25 (m, | |
| | | | | 1 H) 1.92-2.07 (m, 1 H) | |

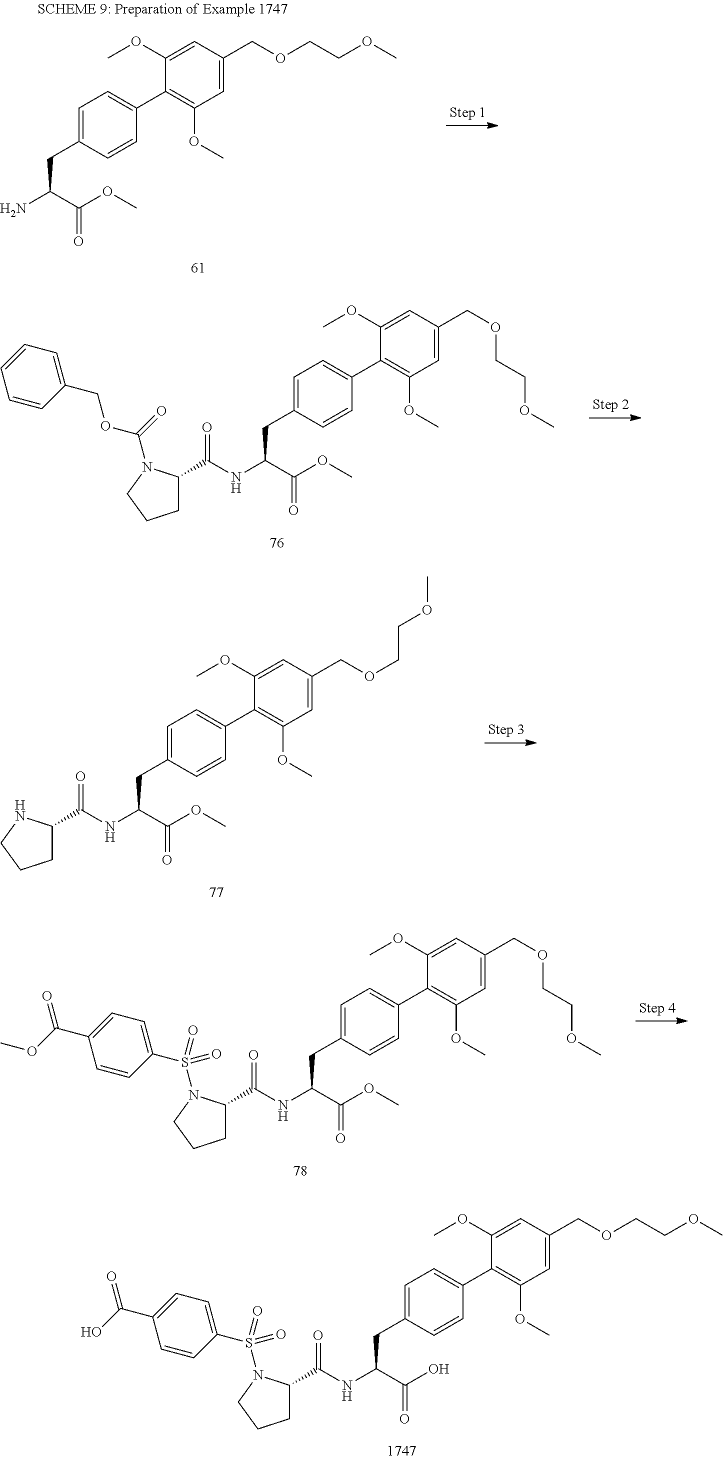

### Scheme 9, Step 1: Preparation of benzyl (S)-2-(((S)-3-(2′,6′-dimethoxy-4′-((2-methoxyethoxy)methyl)-[1,1′-biphenyl]-4-yl)-1-methoxy-1-oxopropan-2-yl)carbamoyl)pyrrolidine-1-carboxylate (76)

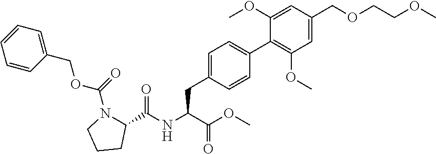 [SMILES: COCCOCc1cc(OC)c(-c2ccc(C[C@H](NC(=O)[C@@H]3CCCN3C(=O)OCc3ccccc3)C(=O)OC)cc2)c(OC)c1]

The compound was prepared from the amide coupling between intermediate 61 and Z-L-proline (46) using General Procedure C of Scheme 2 Step 5. Product benzyl (S)-2-(((S)-3-(2′,6′-dimethoxy-4′-((2-methyl-[1,1′-biphenyl]-4-yl)-1-methoxy-1-oxopropan-2-yl)carbamoyl)pyrrolidine-1-carboxylate (76, 86 mg, 78% yield) was isolated as an oil. LC-MS: t_{R}=2.48 min; m/z=635.2

### Scheme 9, Step 2: Preparation of methyl (S)-3-(2′,6′-dimethoxy-4′-((2-methoxy ethoxy)methyl)-[1,1′-biphenyl]-4-yl)-2-((S)-pyrrolidine-2-carboxamido)propanoate (77)

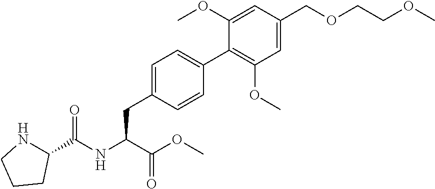 [SMILES: COCCOCc1cc(OC)c(-c2ccc(C[C@H](NC(=O)[C@@H]3CCCN3)C(=O)OC)cc2)c(OC)c1]

The compound was prepared by the hydrogenolysis of intermediate 76 using the procedure of Scheme 5 Step 1. Product methyl (S)-3-(2′,6′-dimethoxy-4′-((2-methoxy ethoxy)methyl)-[1,1′-biphenyl]-4-yl)-2-((S)-pyrrolidine-2-carboxamido)propanoate (77, 64 mg, 94% yield) was isolated as an oil. LC-MS: t_{R}=1.95 min; m/z=501.2.

### Scheme 9, Step 3: Preparation of methyl 4-(((S)-2-(((S)-3-(2′,6′-dimethoxy-4′-((2-methoxyethoxy)methyl)-[1,1′-biphenyl]-4-yl)-1-methoxy-1-oxopropan-2-yl)carbamoyl)pyrrolidin-1-yl)sulfonyl)benzoate (78)

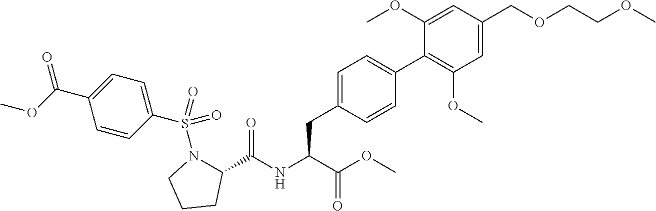

The compound was prepared by the reaction of intermediate 77 with the commercial reagent methyl 4-chlorosulfonylbenzoate using the procedure of Scheme 5 Step 2. Product methyl 4-(((S)-2-(((S)-3-(2′,6′-dimethoxy-4′-((2-methoxyethoxy)methyl)-[1,1′-biphenyl]-4-yl)-1-methoxy-1-oxopropan-2-yl)carbamoyl)pyrrolidin-1-yl)sulfonyl)benzoate (78, 49.6 mg, 55% yield) was isolated as an oil. LC-MS: t_{R}=2.58 min; m/z=699.2.

### Scheme 9, Step 4: Preparation of 4-(((S)-2-(((S)-1-carboxy-2-(2′,6′-dimethoxy-4′-((2-methoxyethoxy)methyl)-[1,1′-biphenyl]-4-yl)ethyl)carbamoyl)pyrrolidin-1-yl) sulfonyl)benzoic Acid (1747)

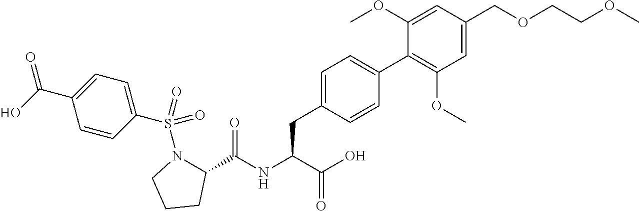 [SMILES: COCCOCc1cc(OC)c(-c2ccc(C[C@H](NC(=O)[C@@H]3CCCN3S(=O)(=O)c3ccc(C(=O)O)cc3)C(=O)O)cc2)c(OC)c1]

The compound was prepared by the hydrolysis of intermediate 78 using General Procedure D of Scheme 2 Step 6. Example 4-(((S)-2-(((S)-1-carboxy-2-(2′,6′-dimethoxy-4′-((2-methoxyethoxy)methyl)-[1,1′-biphenyl]-4-yl)ethyl)carbamoyl)pyrrolidin-1-yl)sulfonyl)benzoic acid (1747, 29.8 mg, 62% yield) was isolated as a white solid. LC-MS: t_{R}=2.0⁹ min; m/z=671.3. ¹H NMR (400 MHz, DMSO-d6) δ ppm 13.20 (br. s, 2H) 8.19 (d, J=8.07 Hz, 1H) 8.03-8.12 (m, 2H) 7.86-7.91 (m, 2H) 7.23 (d, J=8.31 Hz, 2H) 7.11 (d, J=8.07 Hz, 2H) 6.67 (s, 2H) 4.43-4.53 (m, 3H) 4.18 (dd, J=7.09, 3.91 Hz, 1H) 3.61 (s, 6H) 3.56-3.60 (m, 2H) 3.47-3.53 (m, 2H) 3.27 (s, 3H) 3.07-3.22 (m, 3H) 3.02 (m, J=9.00 Hz, 1H) 1.54-1.67 (m, 3H) 1.40-1.52 (m, 1H).

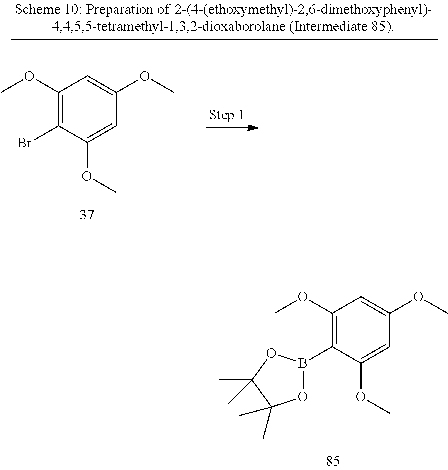

### Scheme 10, Step 1: Preparation of 2-(4-(ethoxymethyl)-2,6-dimethoxyphenyl)-4,4,5,5-tetramethyl-1,3,2-dioxaborolane (85)

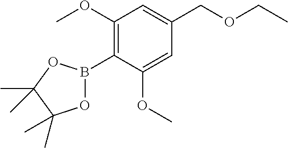 [SMILES: CCOCc1cc(OC)c(B2OC(C)(C)C(C)(C)O2)c(OC)c1]

To a 20 mL microwave vial was added, 2-bromo-5-(ethoxymethyl)-1,3-dimethoxy-benzene (37, 580 mg, 2.11 mmol), bis(pinacolato)diboron (700 mg, 2.74 mmol), Pd(dppf)Cl₂ (95 mg, 0.126 mmol), potassium acetate (621 mg, 6.32 mmol) and 1,4-dioxane (5 mL). The vessel was sparged with nitrogen for 10 minutes and then heated overnight at 90° C. An additional equivalent of bis(pinacolato)diboron (533 mg, 2.11 mmol) and additional Pd(dppf)Cl₂ (95 mg, 0.126 mmol) were added, the vessel was sparged with nitrogen, sealed and heated for three days at 90° C. The reaction was cooled, filtered through Celite® (rinsed with EtOAc) and concentrated in vacuo. The resulting 2.14 g of crude product was chromatographed on a 24 g silica gel column (linear gradient from pure hexanes to pure EtOAc) to give the title compound (85, 342 mg, 1.06 mmol, 50% yield). This material was used as-is. LC-MS: t_{R}=1.755 min; m/z=323 (M+H).

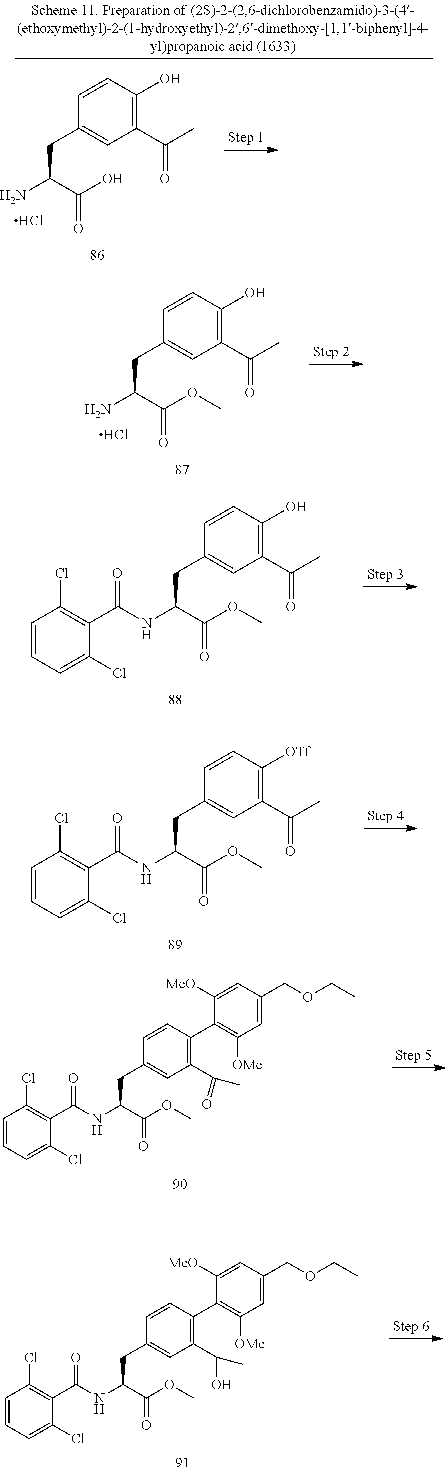

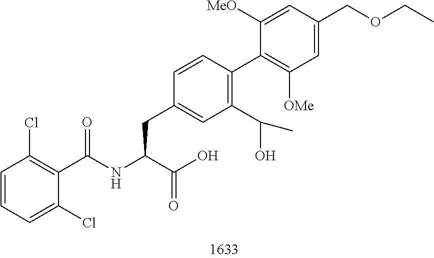 [SMILES: CCOCc1cc(OC)c(-c2ccc(C[C@H](NC(=O)c3c(Cl)cccc3Cl)C(=O)O)cc2C(C)O)c(OC)c1]

### Scheme 11, Step 1: Methyl (S)-3-(3-acetyl-4-hydroxyphenyl)-2-aminopropanoate Hydrochloride (87)

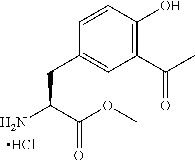 [SMILES: Cl ; COC(=O)[C@@H](N)Cc1ccc(O)c(C(C)=O)c1]

(S)-3-(3-Acetyl-4-hydroxyphenyl)-2-aminopropanoic acid hydrochloride (86, 4.64 g, 17.9 mmol) was dissolved in MeOH (50 mL) and cooled to 0° C. Thionyl chloride (6.5 mL, 89.3 mmol) was slowly added and the reaction was heated to reflux for 1 h. The reaction was cooled to room temperature and concentrated in vacuo. Excess thionyl chloride was azeotroped with methanol to give the crude title product (87, 4.76 g, 17.4 mmol, 97%) as a red solid. LC-MS t_{R}=1.36 min; m/z=238 (M+H).

### Scheme 11, Step 2: Methyl (S)-3-(3-acetyl-4-hydroxyphenyl)-2-(2,6-dichlorobenzamido)propanoate (88)

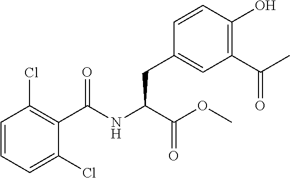 [SMILES: COC(=O)[C@H](Cc1ccc(O)c(C(C)=O)c1)NC(=O)c1c(Cl)cccc1Cl]

To a 50 mL round bottom flask was added 2,6-dichlorobenzoic acid (748 mg, 3.84 mmol), HBTU (1.52 g, 4.02 mmol), HOBt (112 mg, 0.731 mmol) and DMSO (10 mL). DIEA (1.9 mL, 11.0 mmol) was added, and the solution was stirred for 20 minutes. Methyl (S)-3-(3-acetyl-4-hydroxyphenyl)-2-aminopropanoate hydrochloride (87, 1.00 g, 3.65 mmol) was added and the reaction was stirred at room temp for 1 h. The reaction was taken up in EtOAc and washed with dilute HCl, water, then brine. The EtOAc layer was dried (sodium sulfate), filtered and evaporated. The resulting 1.83 g of material was chromatographed on 24 g of silica gel (linear gradient from pure hexanes up to pure EtOAc) to provide the title compound as a white foam (88, 1.14 g, 2.78 mmol, 76%). LC-MS: t_{R}=2.24 min; m/z=432, 434 (M+H, dichloro isotopic pattern). ¹H NMR (400 MHz, DMSO-d₆) δ ppm 11.85 (s, 1H), 9.22 (d, J=8.31 Hz, 1H), 7.83 (d, J=1.96 Hz, 1H), 7.40-7.51 (m, 4H), 6.88 (d, J=8.56 Hz, 1H), 4.75 (m, J=10.30, 8.30, 4.90 Hz, 1H), 3.68 (s, 3H), 3.12 (dd, J=14.06, 4.77 Hz, 1H), 2.91 (dd, J=13.94, 10.27 Hz, 1H), 2.61 (s, 3H).

### Scheme 11, Step 3: Methyl (S)-3-(3-acetyl-4-(((trifluoromethyl)sulfonyl)oxy)phenyl)-2-(2,6-dichlorobenzamido)propanoate (89)

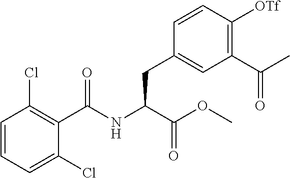 [SMILES: COC(=O)[C@H](Cc1ccc(OS(=O)(=O)C(F)(F)F)c(C(C)=O)c1)NC(=O)c1c(Cl)cccc1Cl]

To a 100 mL round bottom flask was added methyl (S)-3-(3-acetyl-4-hydroxyphenyl)-2-(2,6-dichlorobenzamido)propanoate (88, 1.00 g, 2.44 mmol) and dichloromethane (10 mL). The solution was cooled to −10° C. and pyridine (0.50 mL, 6.09 mmol) was added followed by the drop-wise addition of triflic anhydride (2.9 mL of a 1M solution in dichloromethane, 2.93 mmol). The reaction was allowed to warm to room temperature for 2 h. The reaction was diluted with dichloromethane and washed with citric acid (40 mL of a 10% solution). The DCM layer was dried (sodium sulfate), filtered and evaporated to furnish the crude title compound as an orange foam (89, 1.33 g, 2.45 mmol, quant.). LC-MS: t_{R}=2.48 min; m/z=542, 544 (M+H, dichloro isotopic pattern). The material was carried forward without further purification.

### Scheme 11, Step 4: Methyl (S)-3-(2-acetyl-4′-(ethoxymethyl)-2′,6′-dimethoxy-[1,1′-biphenyl]-4-yl)-2-(2,6-dichlorobenzamido)propanoate (90)

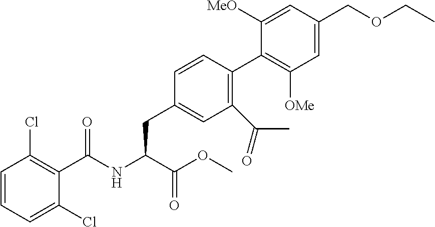 [SMILES: CCOCc1cc(OC)c(-c2ccc(C[C@H](NC(=O)c3c(Cl)cccc3Cl)C(=O)OC)cc2C(C)=O)c(OC)c1]

To a 20 mL microwave vial was added methyl (S)-3-(3-acetyl-4-(((trifluoromethyl)sulfonyl)oxy)phenyl)-2-(2,6-dichlorobenzamido)propanoate (89, 500 mg, 0.922 mmol), 2-(4-(ethoxymethyl)-2,6-dimethoxyphenyl)-4,4,5,5-tetramethyl-1,3,2-dioxaborolane (85, 342 mg, 1.06 mmol), Pd(PPh₃)₄(55 mg, 0.0461 mmol), cesium acetate (535 mg, 2.77 mmol), dioxane (5 mL) and water (1 mL). The vial was crimped shut and sparged with nitrogen for 10 minutes and then heated overnight at 110° C. The reaction was taken up in EtOAc and washed using water, then brine. The EtOAc layer was dried (sodium sulfate), filtered and evaporated. The resulting 1.06 g of orange oil was chromatographed on 12 g of silica gel (linear gradient from pure hexanes up to pure EtOAc). The title compound was isolated as an amber oil (90, 150 mg, 0.255 mmol, 27.6% yield). LC-MS: t_{R}=2.51 min; m/z=588, 590 (M+H, dichloro isotopic pattern). ¹H NMR (400 MHz, DMSO-d₆) δ ppm 9.25 (d, J=8.31 Hz, 1H), 7.60 (d, J=1.71 Hz, 1H), 7.36-7.48 (m, 4H), 7.09 (d, J=7.82 Hz, 1H), 6.66 (s, 2H), 4.79-4.88 (m, 1H), 4.47 (s, 2H), 3.71 (s, 3H), 3.63 (d, J=2.20 Hz, 6H), 3.53 (q, J=7.09 Hz, 2H), 3.23 (dd, J=1.00 Hz, 1H), 3.00 (dd, J=11.20 Hz, 1H), 2.14 (s, 3H), 1.19 (t, J=7.34 Hz, 3H).

### Scheme 11, Step 5: Methyl (2S)-2-(2,6-dichlorobenzamido)-3-(4′-(ethoxymethyl)-2-(1-hydroxyethyl)-2′,6′-dimethoxy-[1,1′-biphenyl]-4-yl)propanoate (91)

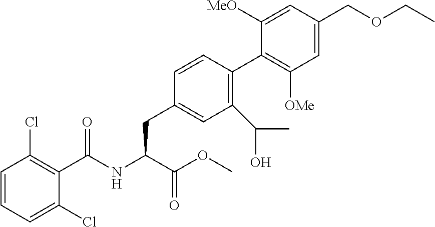 [SMILES: CCOCc1cc(OC)c(-c2ccc(C[C@H](NC(=O)c3c(Cl)cccc3Cl)C(=O)OC)cc2C(C)O)c(OC)c1]

To a round bottom flask was added methyl (S)-3-(2-acetyl-4′-(ethoxymethyl)-2′,6′-dimethoxy-[1,1′-biphenyl]-4-yl)-2-(2,6-dichlorobenzamido)propanoate (90, 150 mg, 0.255 mmol) and MeOH (4 mL). The reaction was cooled to 0° C. and NaBH₄ (15 mg, 0.382 mmol) was added. The reaction was stirred at room temperature. Additional 15 mg aliquots of NaBH₄ were added at 30 min intervals until the starting material was consumed. The reaction was poured into a 5% HCl (10 mL) solution and extracted using dichloromethane (3×50 mL). The organic layer was dried (sodium sulfate), filtered, and evaporated. The resulting 0.159 g of material was chromatographed on 12 g of silica gel (linear gradient from pure hexanes up to pure EtOAc). The title compound (91, 71 mg, 0.120 mmol, 47% yield) was isolated as a clear oil. LC-MS: t_{R}=2.39 min; m/z=612, 614 (M+Na, dichloro isotopic pattern).

### Scheme 11, Step 6: (2S)-2-(2,6-dichlorobenzamido)-3-(4′-(ethoxymethyl)-2-(1-hydroxyethyl)-2′,6′-dimethoxy-[1,1′-biphenyl]-4-yl)propanoic Acid (1633)

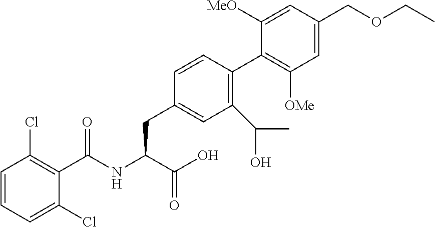 [SMILES: CCOCc1cc(OC)c(-c2ccc(C[C@H](NC(=O)c3c(Cl)cccc3Cl)C(=O)O)cc2C(C)O)c(OC)c1]

To a round bottom flask was added methyl (2S)-2-(2,6-dichlorobenzamido)-3-(4′-(ethoxymethyl)-2-(1-hydroxyethyl)-2′,6′-dimethoxy-[1,1′-biphenyl]-4-yl)propanoate (91, 71 mg, 0.120 mmol), acetonitrile (1 mL) and water (1 mL). Lithium hydroxide hydrate (15 mg, 0.361 mmol) was added and the reaction was stirred at room temperature for 1 h. The acetonitrile was evaporated and the reaction was acidified with 2N HCl. The mixture was evaporated to dryness and chromatographed on a 15.5 g C18 RediSep Gold cartridge (linear gradient from 10% MeCN/water up to pure MeCN with 0.1% formic acid as modifier). The title compound was isolated as a white solid (1633, 54.0 mg, 0.0937 mmol, 78% yield). LC-MS: t_{R}=2.27 min; m/z=598, 600 (M+Na, dichloro isotopic pattern). ¹H NMR (400 MHz, DMSO-d₆) δ ppm 12.80 (br. s., 1H), 9.10 (t, J=8.68 Hz, 1H), 7.50 (dd, J=7.09, 1.47 Hz, 1H), 7.32-7.46 (m, 3H), 7.03-7.20 (m, 1H), 6.78 (dd, J=7.70, 5.75 Hz, 1H), 6.67 (s, 2H), 4.64-4.83 (m, 2H), 4.48 (s, 2H), 4.35 (m, J=6.40, 3.20, 3.20 Hz, 1H), 3.63 (s, 6H), 3.54 (q, J=7.01 Hz, 2H), 3.11-3.24 (m, 1H), 2.93 (dd, J=14.06, 9.90 Hz, 1H), 1.20 (t, J=7.09 Hz, 3H), 1.02 (dd, J=7.70, 6.48 Hz, 3H).

### Biological Assay Results

Flow Cytometry Cell-Based Assay.

Compounds were tested for their ability to inhibit the binding of soluble VCAM-1 to human G2 acute lymphoblastic leukemia (ALL) cells. Briefly, G2 ALL cells are pre-incubated with increasing concentrations (0.001 to 1000 nM) of compounds for 30 minutes. Soluble VCAM/Fc chimera protein (R&D systems) is then added to the mixture and the cells incubated for an additional 30 minutes. Afterwards, cells are washed and VCAM-1 is detected using PE-conjugated secondary mAbs. In each experiment, an aliquot of cells will be stained with isotype control mAbs to serve as a negative control. The percentage of VCAM-1 binding cells was then determined by flow cytometry.

TABLE 8
Inhibition of sVCAM-1 binding to human G2 ALL cells.

| Example | sVCAM-1 IC₅₀ (nM) |
|---|---|
| Firategrast | 230 |
| 1219 | 72 |
| 1220 | 0.47 |
| 1221 | 0.58 |
| 1222 | 218 |
| 1223 | 70 |
| 1224 | 1.57 |
| 1554 | 1.47 |
| 1555 | 12.9 |
| 1570 | 2.97 |
| 1571 | 4.43 |
| 1608 | 2.47 |
| 1609 | 11.7 |
| 1610 | 0.83 |
| 1611 | 0.49 |
| 1633 | 3.33 |
| 1745 | 3.70 |
| 1746 | 3.79 |
| 1747 | 7.58 |
| 2087 | 2.06 |
| 2088 | 0.70 |

Mice.

DBA/2J mice were purchased from the Jackson Laboratory (Bar Harbor, Me., USA). Animals were housed at the Washington University Medical School vivarium under SPF conditions. All experiments were performed in accordance with the guidelines of the Washington University Animal Studies Committee and the institutional animal care and use committee (IACUC), in agreement with AAALAC guidelines.

HSPC Mobilization.

The VLA-4 inhibitors Examples 1220, 1221, 1224, 1610, and 1611 were prepared in DMSO/10 mM sodium bicarbonate pH 8/saline (1%/49.5%/49.5%) and injected subcutaneously (SC) at a dose of 3 mg/kg.

Colony Forming Cell (CFC) Assay.

Peripheral blood (PB) was drawn from the facial vein without anaesthesia into K/EDTA anti-coagulated tubes (Sarstedt AG & Co, Numbrecht, Germany). Red blood cells were removed from 25 L aliquots of blood using hypotonic lysis (Ammonium-Chloride-Potassium, ACK buffer, 5-10 min at RT) and samples were mixed with 2.5 mL methylcellulose media supplemented with a cocktail of recombinant cytokines (MethoCult 3434; Stem Cell Technologies, Vancouver, BC, Canada). Cultures were plated in duplicate in 35 mm dishes and placed in a humidified chamber with 5% CO₂ at 37° C. After 7 d of culture, colonies containing at least 50 cells were counted using an inverted microscope in a blinded fashion.

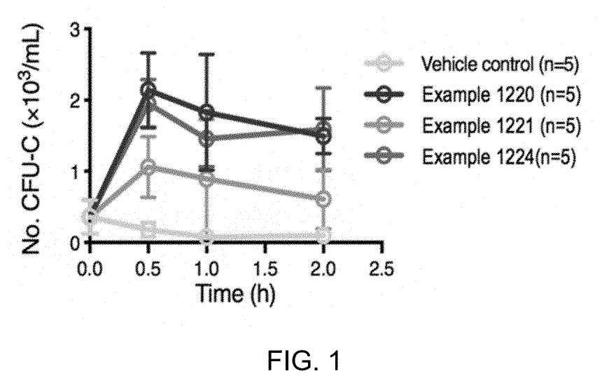

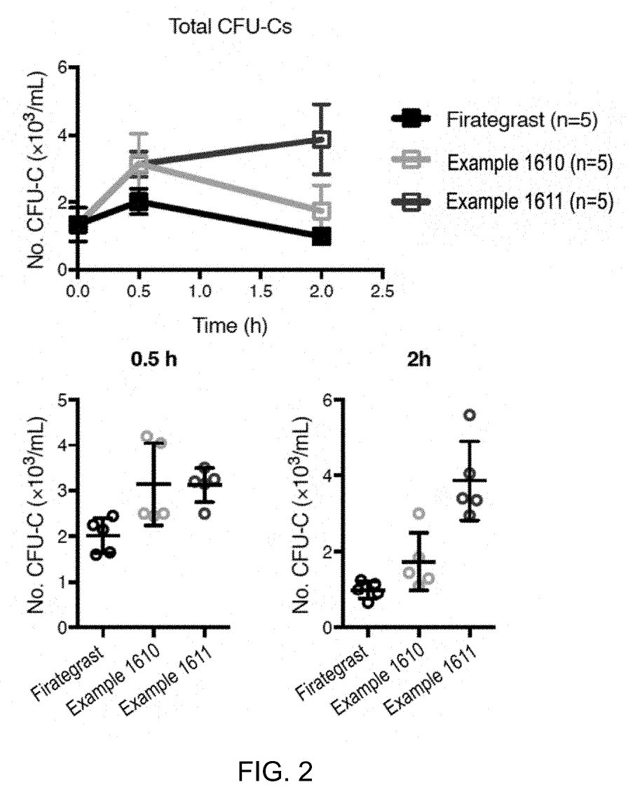

Results for Examples 1220, 1221, and 1224 are shown in FIG. 1. Results for Examples 1610 and 1611 are shown in FIG. 2.

### Example 2 (Section II—Integrin Inhibitors in Combination with an Agent which Interacts with a Chemokine Receptor)

In Vitro Assays of HSPC Mobilization.

Colony Assays.

In short-term colony-forming cell (CFC) assays, test samples are cultured in a semi-solid matrix supplemented with nutrients and cytokines for −1 week at 37° C. During this culture period, CFC proliferate and produce discrete cell clusters or colonies of morphologically recognizable daughter cells that can be quantified by light microscopy. Here, mice will be treated with mobilization regimens and peripheral blood will be collected via the retroorbital sinus at the peak of mobilization. After collection of blood, a complete blood count will be performed using a Hemavet 950FS cell counter (Drew Scientific) and CFC assays will be established in methylcellulose-containing Iscove modified Dulbecco medium supplemented with interleukin-3, interleukin-6, and stem cell factor (MethoCult3534; StemCell Technologies). CPU-GM, BFU-E, and CFU-GEMM colony types will be enumerated following 1 week of culture at 37° C.

Flow Cytometric Enumeration of Murine HSPCs.

Among the subsets that define HSPCs, CD34⁻ c-kit⁺ Sca-1⁺ lineage marker (CD34⁻KSL) cells are regarded as one of the populations that have the highest enrichment of HSPCs in adult mouse bone marrow. More recently, the SLAM family markers, CD150 and CD48, have been used to differentiate stem cells from more committed progenitor cells. Here, primitive murine HSPC mobilization will be examined by flow cytometry enumeration of CD34⁻c-kit⁺Sca-1⁺ lineage marker⁻CD150⁺CD48⁻ cell numbers following treatment with mobilization regimens.

In Vivo Assays of HSPC Mobilization.

Competitive Repopulation Assays.

The definitive assay for stem cell activity in a test sample is the complete and sustained (>5 months) reconstitution of all hematopoietic lineages in irradiated recipients by transplanted HSPCs. For competitive stem cell repopulation assays, peripheral blood mononuclear cells (PBMCs) from wild-type C57BL/6 (CD45.2⁺) mice mobilized by the regimens described herein will be mixed with 2×10⁵ competitor bone marrow cells from C57BL/6J×B6.SJL-Ptprca Pepcb/BoyJ F1 (CD45.1⁺/CD45.2⁺) mice and transplanted into lethally irradiated (1100 cGy) recipients congenic at the CD45.1⁺ locus (B6.SJL-Ptprca Pepcb/BoyJ). At least 3 mobilized PBMC:competitor ratios (typically the number of PBMCs isolated from 0.5, 1, and 1.5 mL of blood) will be evaluated to establish the appropriate donor:competitor ratio. Secondary transplantations will be performed by intravenously injecting 10⁶ unfractionated bone marrow cells from donors 5 months after transplantation into lethally irradiated (1100 cGy) CD45.1⁺ secondary recipients.

In Vitro Assay of Chemosensitization.

Stromal Protections Assay.

The impact of bone marrow stroma on leukemia cell survival during treatments listed herein will be assessed. Mouse AML (APL) and human ALL (G2) cell lines will be incubated with the appropriate drugs and drug combinations±chemotherapy±stroma. BLI (bioluminescence) as well as standard MTT assays will be used to assess cell growth and survival.

In Vivo Assay of Chemosensitization.

Recipient mice (C57BL/6) will be injected with 1×10⁶ murine acute promyelocytic (APL) leukemia cells transduced with Click Beetle Red-GFPe. In the absence of treatment all mice develop overt leukemia by day +20 and die from a rapidly fatal leukemia with leukocytosis and splenomegaly by day +35. The treatments disclosed herein will be administered with and without chemotherapy and mouse survival and leukemia burden as measured by BLI and FACS (Gr-1+/c-kit+/CD34+ leukemia cells) will be assessed. Both BLI and FACS measurements will be obtained on days +7, +14, +21, +28, and +35.

C57BL/6 into BALB/c Transplant Model.

Lethal Irradiation of BALB/c Recipients.

Recipient BALB/c mice are lethally irradiated with 925 cGy of total body irradiation on day −1 and then infused with 5×10⁶ T cell depleted bone marrow cells (TCD-BM) containing 2×10⁶ T cells from C57BL/6 mice on day 0. Mice are monitored for indications of GvHD (clinical score based on weight loss, hunched posture, activity, fur texture, diarrhea and skin integrity) and survival time. Signs of GvHD are typically seen within 2-3 weeks and all mice die by 4-5 weeks. The rate of GvHD in irradiated mice receiving TCD-BM only (n=15 mice) will be compared with the rate in mice receiving TCD-BM plus the different donor PBMC populations described herein. Mice will be sacrificed on day 25 to collect and examine GvHD target organs (histology of liver, intestines, and spleen). Peripheral blood will also be collected on day 25 to determine the extent of donor cell engraftment and immune reconstitution (flow cytometry using antibodies against CD3, B220, CD4, CD8, FOXP3, H-2Kb, CD45.1, and CD45.2).

Sublethal Irradiation of BALB/c Recipients.

Recipient BALB/c mice are sublethally irradiated with 500 cGy of total body irradiation on day and then infused with 5×10⁶ T cell depleted bone marrow cells (TCD-BM) containing 2×10⁶ T cells from C57BL/6 mice on day 0. Mice are monitored for indications of GvHD and donor engraftment as described above.

C57BL/6 into B6D2F1 Transplant Model.

Lethal Irradiation of B6D2F1 Recipients.

Recipient B6D2F1 mice are lethally irradiated with 1100 cGy (split doses, 3 hr apart) of total body irradiation on day −1 and then infused with 1×10 T cell depleted splenocytes (TCD-SPL) containing 3×10⁶ T cells from C57BL/6 mice on day 0. The rate of GvHD and donor engraftment in irradiated mice receiving TCD-SPL only (n=15 mice) will be compared with the rate in mice receiving TCD-SPL plus the different purified T cell populations listed herein. Mice will be monitored for indications of GvHD and donor engraftment as described above.

VLA-4 Antagonists Enhance Mobilization of HSPCs.

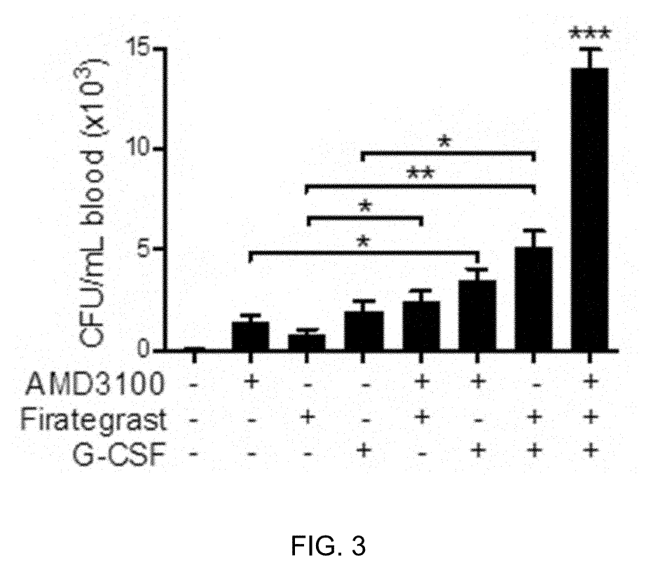

Treatment of mice with the VLA-4 antagonist BIO5192 results in a 30-fold increase in mobilization of HSPCs and combination of BIO5192 with plerixafor gave a further 3-fold increase. Treatment with a combination of BIO5192, plerixafor, and G-CSF enhanced mobilization by 17-fold compared to G-CSF alone. BIO5192 also mobilized long-term repopulating cells that successfully engraft and expand in a multi-lineage manner in secondary transplant experiments. Similar mobilization studies were performed using the small molecule VLA-4 antagonist firategrast. Similar results relative to BIO5192 were obtained (FIG. 3). Although significant additive/synergistic effects were observed following combination therapy with firategrast and AMD3100 and/or G-CSF, 30-fold higher concentrations of firategrast were required to mimic the mobilization effects of BIO5192 (100 mg/kg vs. 3 mg/kg, respectively). This decreased biological activity is presumably due to firategrast's ˜30-fold lower potency for VLA-4.

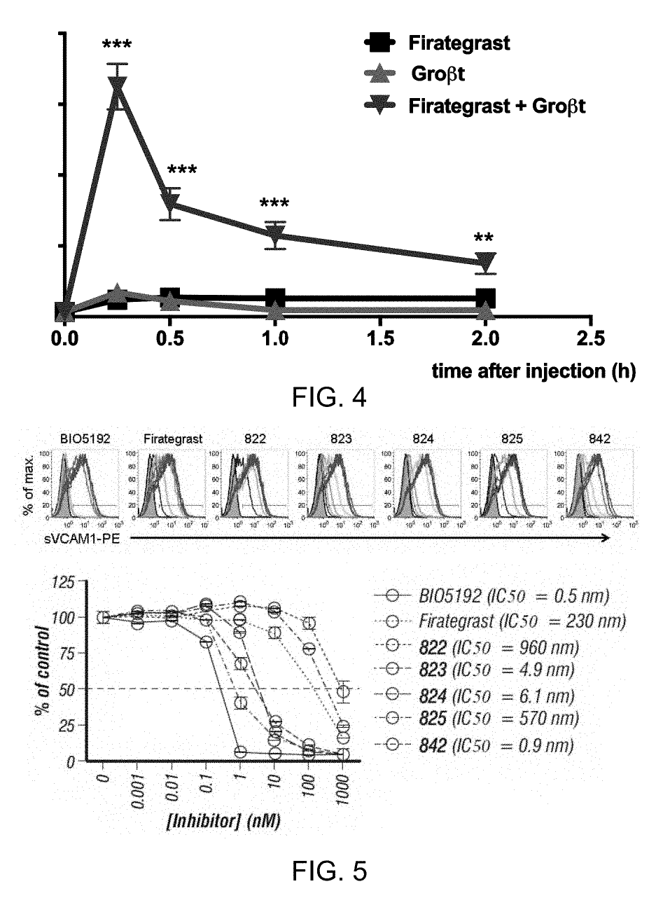

The combination of firategrast with the CXCR2 agonist truncated Gro-beta (Gro-βt, SB-251353) was also tested. Gro-βt is a recombinant, N-terminal truncated form of Gro-β that binds to CXCR2 with greater potency than the full-length form of Gro-β. If both firategrast and Gro-βt are given to mice within 5 minutes of each other and blood is collected 15 min post-administration, a significant synergy in colony-forming cell (CFC) mobilization is observed that persisted for >2 hours (FIG. 4). Remarkably, the level of CFC mobilization achieved with the firategrast and Gro-βt regimen is greater than that of G-CSF used alone for 4 days.

Combination of Groβ or Truncated Groβ (Groβt) Plus a VLA-4 Small Molecule Inhibitor for Hematopoietic Stem Cell Mobilization and Leukemia Chemosensitization. Mobilization of HSPCs with Groβt and Firategrast

This experiment showed that the combination of Groβt (2.5 mg/kg, SC) and firategrast (VLA-4 inhibitor; 100 mg/kg, IV) synergistically mobilized murine HSPCs, as measured by a colony-forming unit (CFU) assay of mobilized peripheral blood. The combination of Groβt and firategrast mobilized 5.6-fold more murine CFUs (14,210±843 CFU/mL blood) compared to when each agent was administered alone (Groβt: 2,520±580 CFU/mL; firategrast: 2,480±230 CFU/mL) in DBA/2 mice. This experiment was carried out again and similar results were obtained. In the second experiment, the combination of Groβt (2.5 mg/kg, SC) and firategrast (100 mg/kg, IV) mobilized 9.5-fold more murine CFUs (16,225±3,600 CFUs) compared to when each agent was administered alone (Groβt: 1,710±600 CFU/mL; firategrast: 1,205±290 CFU/mL) in DBA/2 mice. To ensure the synergistic CFU mobilization that was observed with the combination of Groβt and firategrast in DBA/2 mice in the previous experiments, the administration of these two agents was also tested in a different mouse strain. Specifically, the combination of Groβt (2.5 mg/kg, SC) and firategrast (100 mg/kg, IV) mobilized 7.3-fold more murine CFUs (4,600±1,600 CFU/mL) compared to when each agent was administered alone (Groβt: 630±390 CFU/mL; firategrast: 430±130 CFU/mL) to C57BL/6 mice. Furthermore, the combination of Groβt and firategrast mobilized 18-fold more murine lineage-Sca-1+c-kit+(LSK) cells (72 LSK cells/μL blood), another measure of HSPCs, compared to when each agent was administered alone (Groβt: 4 LSK cells/μL; firategrast: 3 LSK cells/μL) to C57BL/6 mice.

Additional VLA-4 Antagonists. Preparation of Compounds

Table 9 below includes the characterization of some of the VLA-4 antagonist described herein. Synthesis of these compounds is described in PCT/US2017/059733, which is incorporated herein by reference.

TABLE 9
Example Compounds and Characterization

| Ex. | | | | | LC/MS |
|---|---|---|---|---|---|
| No. | IUPAC Name | Structure | Yield | ¹H NMR δ | m/z |
| Cmpd 1633 | (2S)-2-(2,6-dichlorobenzamido)-3-(4′- (ethoxymethyl)-2-(1-hydroxyethyl)-2′,6′- dimethoxy-[1,1′-biphenyl]-4-yl)propanoic acid | 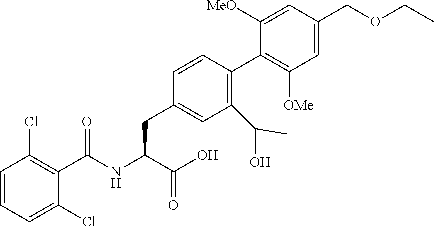 [SMILES: CCOCc1cc(OC)c(-c2ccc(C[C@H](NC(=O)c3c(Cl)cccc3Cl)C(=O)O)cc2C(C)O)c(OC)c1] | 78% | 12.80 (br. s., 1 H), 9.10 (t, J = 8.68 Hz, 1 H), 7.50 (dd, J = 7.09, 1.47 Hz, 1 H), 7.32-7.46 (m, 3 H), 7.03- 7.20 (m, 1 H), 6.78 (dd, J = 7.70, 5.75 Hz, 1 H), 6.67 (s, 2 H), 4.64- 4.83 (m, 2 H), 4.48 (s, 2 H), 4.35 (m, J = 6.40, 3.20, 3.20 Hz, 1 H), 3.63 (s, 6 H), 3.54 (q, J = 7.01 Hz, 2 H), 3.11-3.24 (m, 1 H), 2.93 (dd, J = 14.06, 9.90 Hz, 1 H), 1.20 (t, J = 7.09 Hz, 3 H), 1.02 (dd, J = 7.70, 6.48 Hz, 3 H). | 598, 600 |
| Cmpd 1219 | 4-{[(1S)-2-[4-(4-carbamoyl-2,6- dimethoxyphenyl)phenyl]-1- carboxyethyl]carbamoyl}-3,5- dichlorobenzoic acid | 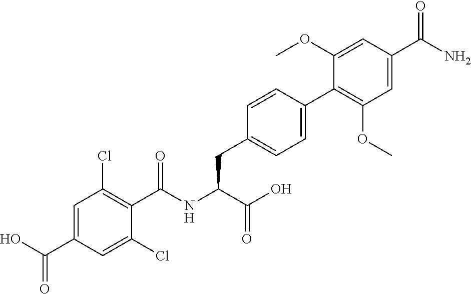 [SMILES: COc1cc(C(N)=O)cc(OC)c1-c1ccc(C[C@H](NC(=O)c2c(Cl)cc(C(=O)O)cc2Cl)C(=O)O)cc1] | 59% | 13.10 (br. s, 2H) 9.20 (d, J = 8.31 Hz, 1 H) 7.83 (s, 2 H) 7.30 (d, J = 8.07 Hz, 2 H) 7.27 (s, 2 H) 7.13 (d, J = 8.07 Hz, 2 H) 4.72-4.82 (m, 1 H) 3.72 (s, 6 H) 3.33 (br. s, 2 H) 3.22 (dd, J = 13.94, 4.40 Hz, 1 H) 2.92 (dd, J = 14.06, 10.64 Hz, 1 H) | 562.0, 564.0 |
| Cmpd 1220 | 4-{[(1S)-1-carboxy-2-{4-[4- (hydroxymethyl)-2,6- dimethoxyphenyl]phenyl}ethyl]carbamoyl}- 3,5-dichlorobenzoic acid | 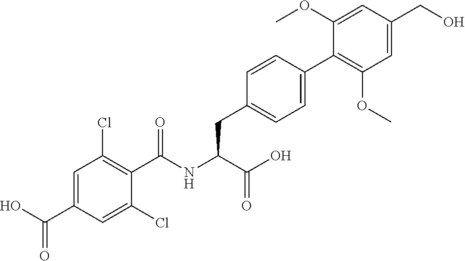 [SMILES: COc1cc(CO)cc(OC)c1-c1ccc(C[C@H](NC(=O)c2c(Cl)cc(C(=O)O)cc2Cl)C(=O)O)cc1] | 41% | 13.72 (br. s, 1 H) 12.90 (br. s, 1 H) 9.22 (d, J = 8.31 Hz, 1 H) 7.85 (s, 2 H) 7.26 (d, J = 8.07 Hz, 2 H) 7.08 (d, J = 8.07 Hz, 2 H) 6.69 (s, 2 H) 5.24 (br. s, 1 H) 4.66-4.82 (m, 1 H) 4.52 (s, 2 H) 3.65 (s, 6 H) 3.20 (dd, J = 14.18, 4.65 Hz, 1 H) 2.91 (dd, J = 14.31, 10.64 Hz, 1H) | 548.0, 550.0 |
| Cmpd 1221 | 4-{[(1S)-1-carboxy-2-{4-[4-(ethoxymethyl)- 2,6- dimethoxyphenyl]phenyl}ethyl]carbamoyl}- 3,5-dichlorobenzoic acid | 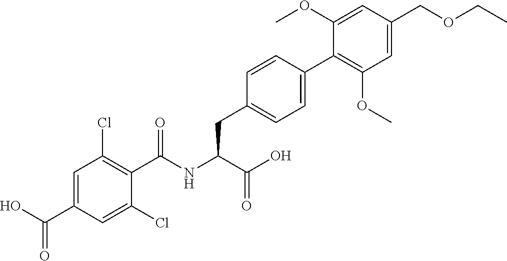 [SMILES: CCOCc1cc(OC)c(-c2ccc(C[C@H](NC(=O)c3c(Cl)cc(C(=O)O)cc3Cl)C(=O)O)cc2)c(OC)c1] | 52% | 13.74 (br. s, 1 H) 12.87 (br. s, 1 H) 9.22 (d, J = 8.31 Hz, 1 H) 7.85 (s, 2 H) 7.26 (d, J = 8.31 Hz, 2 H) 7.09 (d, J = 8.07 Hz, 2 H) 6.68 (s, 2 H) 4.76 (m, J = 2.20 Hz, 1 H) 4.47 (s, 2 H) 3.66 (s, 6 H) 3.53 (q, J = 7.09 Hz, 2 H) 3.20 (dd, J = 13.94, 4.40 Hz, 1 H) 2.91 (dd, J = 14.18, 10.27 Hz, 1 H) 1.19 (t, J = 6.97 Hz, 3 H) | 576.1, 578.0 |
| Cmpd 1222 | 4-{4-[(2S)-2-carboxy-2-[(4-carboxy-2,6- dichlorophenyl)formamido]ethyl]phenyl}- 3,5-dimethoxybenzoic acid | 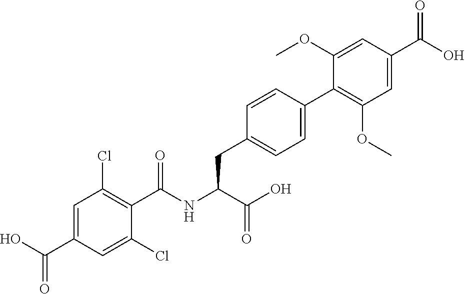 [SMILES: COc1cc(C(=O)O)cc(OC)c1-c1ccc(C[C@H](NC(=O)c2c(Cl)cc(C(=O)O)cc2Cl)C(=O)O)cc1] | 51% | 13.00 (br. s, 3 H) 9.22 (d, J = 8.31 Hz, 1 H) 7.85 (s, 2 H) 7.30 (d, J = 8.31 Hz, 2 H) 7.27 (s, 2 H) 7.13 (d, J = 8.07 Hz, 2 H) 4.71-4.83 (m, 1 H) 3.72 (s, 6 H) 3.22 (dd, J = 14.31, 4.28 Hz, 1 H) 2.92 (dd, J = 13.94, 10.76 Hz, 1 H) | 562.0, 564.0 |
| Cmpd 1223 | 4-{[(1S)-1-carboxy-2-{4-[4-(2- hydroxypropan-2-yl)-2,6- dimethoxyphenyl]phenyl}ethyl]carbamoyl}- 3,5-dichlorobenzoic acid | 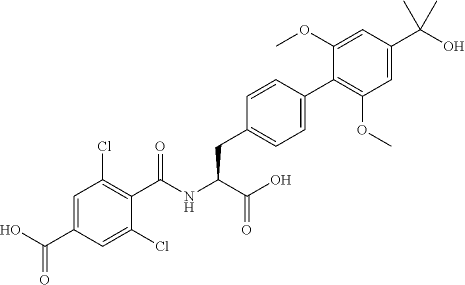 [SMILES: COc1cc(C(C)(C)O)cc(OC)c1-c1ccc(C[C@H](NC(=O)c2c(Cl)cc(C(=O)O)cc2Cl)C(=O)O)cc1] | 74% | 13.72 (br. s., 1 H) 12.88 (br. s., 1 H) 9.22 (d, J = 8.31 Hz, 1 H) 7.85 (s, 2 H) 7.25 (d, J = 8.07 Hz, 2 H) 7.09 (d, J = 8.31 Hz 2 H) 6.81 (s, 2 H) 5.04 (s, 1 H) 4.67-4.82 (m, 1 H) 3.66 (s, 6 H) 3.20 (dd, J = 14.06, 4.03 Hz, 1 H) 2.91 (dd, J = 14.18, 10.51 Hz, 1 H) 1.47(s, 6H) | 576.1, 578.0 |
| Cmpd 1224 | 4-{[(1S)-1-carboxy-2-{4-[4-(fluoromethyl)- 2,6- dimethoxyphenyl]phenyl}ethyl]carbamoyl}- 3,5-dichlorobenzoic acid | 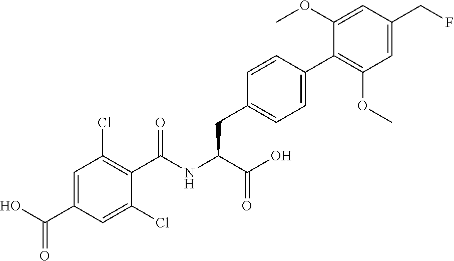 [SMILES: COc1cc(CF)cc(OC)c1-c1ccc(C[C@H](NC(=O)c2c(Cl)cc(C(=O)O)cc2Cl)C(=O)O)cc1] | 69% | 13.73 (br. s, 1 H) 12.89 (br. s, 1 H) 9.22 (d, J = 8.31 Hz, 1 H) 7.85 (s, 2 H) 7.28 (d, J = 8.07 Hz, 2 H) 7.10 (d, J = 8.07 Hz, 2 H) 6.80 (d, J = 1.47 Hz, 2 H) 5.49 (s, 1 H) 5.37 (s, 1 H) 4.77 (m, J = 2.40 Hz, 1 H) 3.68 (s, 6 H) 3.21 (dd, J = 14.18, 3.91 Hz, 1 H) 2.92 (dd, J = 14.06, 10.64 Hz, 1 H) | 550.0, 552.0 |
| Cmpd 1608 | (S)-2-((S)-1-((3,5-dichlorophenyl)sulfonyl)- 2-methylpyrrolidine-2-carboxamido)-3-(4′- (ethoxymethyl)-2′,6′-dimethoxy-[1,1′- biphenyl]-4-yl)propanoic acid | 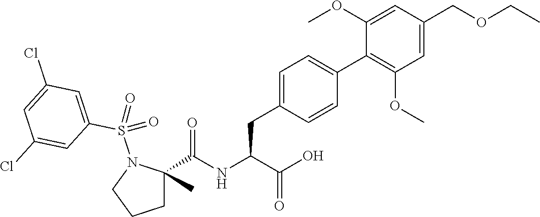 [SMILES: CCOCc1cc(OC)c(-c2ccc(C[C@H](NC(=O)[C@]3(C)CCCN3S(=O)(=O)c3cc(Cl)cc(Cl)c3)C(=O)O)cc2)c(OC)c1] | 66% | ¹H NMR (400 MHz, DMSO- d6) δ ppm 12.86 (br. s., 1 H) 7.97 (t, J = 1.96 Hz, 1 H) 7.81 (d, J = 1.71 Hz, 2 H) 7.70 (d, J = 7.82 Hz, 1 H) 7.20 (d, J = 8.31 Hz, 2 H) 7.09 (d, J = 8.31 Hz, 2 H) 6.66 (s, 2 H) 4.50 (m, J = 4.90 Hz, 1 H) 4.46 (s, 2 H) 3.62 (s, 6 H) 3.51 (q, J = 7.09 Hz, 2 H) 3.37 (m, J = 7.10 Hz, 2 H) 3.12- 3.22 (m, 1 H) 2.96-3.08 (m, 1 H) 1.92 (m, J = 7.30 Hz, 1 H) 1.67-1.80 (m, 2 H) 1.61-1.69 | 679.1, 681.1 |
| | | | | (m, 1 H) 1.49 (s, 3 H) 1.18 (t, | |
| | | | | J = 6.97 Hz, 3 H). | |
| Cmpd 1609 | (S)-3-(4′-(ethoxymethyl)-2′,6′-dimethoxy- [1,1′-biphenyl]-4-yl)-2-((S)-2-methyl-1- (phenylsulfonyl)pyrrolidine-2- carboxamido)propanoic acid | 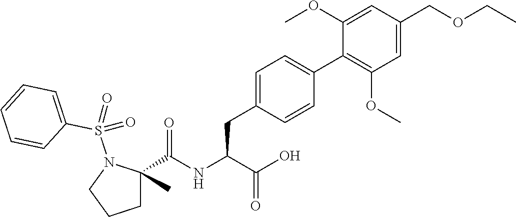 [SMILES: CCOCc1cc(OC)c(-c2ccc(C[C@H](NC(=O)[C@]3(C)CCCN3S(=O)(=O)c3ccccc3)C(=O)O)cc2)c(OC)c1] | 73% | ¹H NMR (400 MHz, DMSO- d6) δ ppm 12.90 (br. s., 1 H) 7.79-7.88 (m, 2 H) 7.61-7.70 (m, 1 H) 7.50-7.60 (m, 3 H) 7.19 (d, J = 8.07 Hz, 2 H) 7.09 (d, J = 8.07 Hz, 2 H) 6.66 (s, 2 H) 4.52 (m, J = 4.90 Hz, 1 H) 4.46 (s, 2 H) 3.62 (s, 6 H) 3.52 (q, J = 7.01 Hz, 2 H) 3.37 (m, J = 5.10 Hz, 1 H) 3.23-3.30 (m, 1 H) 3.19 (dd, J = 13.94, 4.89 Hz, 1 H) 3.02 (dd, J = 1.00 Hz, 1 H) 1.86-1.97 (m, 1 H) 1.49- | 611.2 |
| | | | | 1.71 (m, 3 H) 1.43 (s, 3 H) 1.18 | |
| | | | | (t, J = 6.97 Hz, 3 H). | |
| Cmpd 1610 | (S)-3-(4′-(ethoxymethyl)-2′,6′-dimethoxy- [1,1′-biphenyl]-4-yl)-2-((S)-1- (phenylsulfonyl)pyrrolidine-2- carboxamido)propanoic acid | 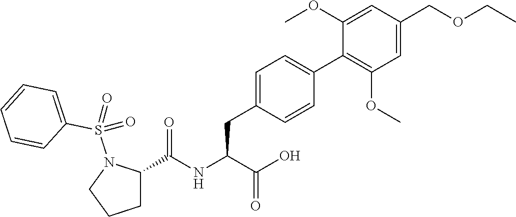 [SMILES: CCOCc1cc(OC)c(-c2ccc(C[C@H](NC(=O)[C@@H]3CCCN3S(=O)(=O)c3ccccc3)C(=O)O)cc2)c(OC)c1] | 93% | ¹H NMR (400 MHz, DMSO- d6) δ ppm 12.90 (br. s., 1 H) 8.17 (d, J = 8.07 Hz, 1 H) 7.82 (d, J = 7.58 Hz, 2 H) 7.65-7.74 (m, 1 H) 7.55-7.64 (m, 2H) 7.22 (d, J = 8.07 Hz, 2 H) 7.10 (d, J = 7.82 Hz, 2 H) 6.66 (s, 2 H) 4.47-4.55 (m, 1 H) 4.45 (s, 2 H) 4.14 (m, J = 7.60 Hz, 1 H) 3.61 (s, 6 H) 3.51 (q, J = 7.09 Hz, 2 H) 3.31-3.38 (m, 1H) 3.07-3.19 (m, 2 H) 2.94-3.05 (m, 1 H) 1.46-1.65 (m, 3 H) | 597.2 |
| | | | | 1.40 (m, J = 6.10 Hz, 1 H) 1.17 | |
| | | | | (t, J = 7.09 Hz, 3 H). | |
| Cmpd 1611 | (S)-2-((S)-1-((3,5- dichlorophenyl)sulfonyl)pyrrolidine-2- carboxamido)-3-(4′-(ethoxymethyl)-2′,6′- dimethoxy-[1,1′-biphenyl]-4-yl)propanoic acid | 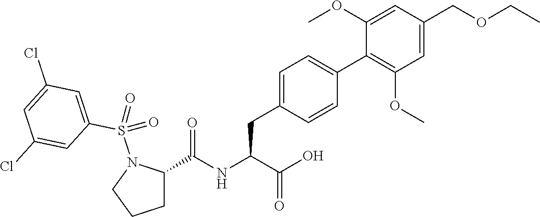 [SMILES: CCOCc1cc(OC)c(-c2ccc(C[C@H](NC(=O)[C@@H]3CCCN3S(=O)(=O)c3cc(Cl)cc(Cl)c3)C(=O)O)cc2)c(OC)c1] | 84% | ¹H NMR (400 MHz, DMSO- d6) δ ppm 12.89 (s, 1 H) 8.31 (d, J = 8.07 Hz, 1 H) 8.02 (s, 1 H) 7.85 (d, J = 1.47 Hz, 2 H) 7.23 (d, J = 7.82 Hz, 2 H) 7.10 (d, J = 7.83 Hz, 2 H) 6.66 (s, 2 H) 4.48-4.57 (m, 1 H) 4.46 (s, 2 H) 4.26-4.34 (m, 1 H) 3.62 (s, 6 H) 3.51 (q, J = 6.93 Hz, 2 H) 3.35-3.41 (m, 1 H) 3.23 (m, J = 9.30 Hz, 1 H) 3.06-3.15 (m, 1 H) 2.94-3.05 (m, 1 H) 1.61- 1.79 (m, 3 H) 1.54 (m, J = 4.90 | 665.1, 667.1 |
| | | | | Hz, 1 H) 1.18 (t, J = 6.97 Hz, 3 | |
| | | | | H). | |
| Cmpd 1745 | (S)-3-(2′,6′-dimethoxy-4′-((2- methoxyethoxy)methyl)-[1,1′-biphenyl]-4- yl)-2-((S)-1-(phenylsulfonyl)azetidine-2- carboxamido)propanoic acid | 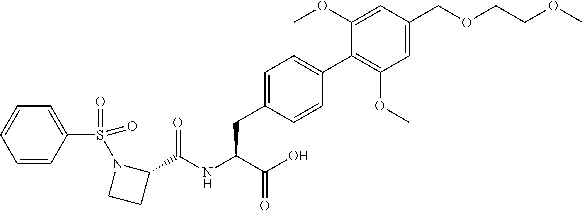 [SMILES: COCCOCc1cc(OC)c(-c2ccc(C[C@H](NC(=O)[C@@H]3CCN3S(=O)(=O)c3ccccc3)C(=O)O)cc2)c(OC)c1] | 62% | ¹H NMR (400 MHz, DMSO- d6) δ ppm 12.97 (br. s, 1 H) 8.18 (d, J = 8.07 Hz, 1 H) 7.81- 7.87 (m, 2 H) 7.74-7.80 (m, 1 H) 7.64-7.70 (m, 2 H) 7.23 (d, J = 8.07 Hz, 2 H) 7.12 (d, J = 8.31 Hz, 2 H) 6.68 (s, 2 H) 4.53 (m, J = 5.40 Hz, 1 H) 4.50 (s, 2 H) 4.30 (dd, J = 9.29, 7.34 Hz, 1 H) 3.67 (m, J = 4.40 Hz, 1 H) 3.62 (s, 6 H) 3.57-3.61 (m, 2 H) 3.55 (d, J = 9.05 Hz, 1 H) 3.48- 3.53 (m, 2 H) 3.27 (s, 3 H) 3.13 | 613.2 |
| | | | | (m, J = 5.10 Hz, 1 H) 3.03 (m, | |
| | | | | J = 8.30 Hz, 1 H) 2.00-2.11 (m, | |
| | | | | 1 H) 1.86-1.98 (m, 1H) | |
| Cmpd 1746 | (S)-2-((S)-1-((3,5- dichlorophenyl)sulfonyl)azetidine-2- carboxamido)-3-(2′,6′-dimethoxy-4′-((2- methoxyethoxy)methyl)-[1,1′-biphenyl]-4- yl)propanoic acid | 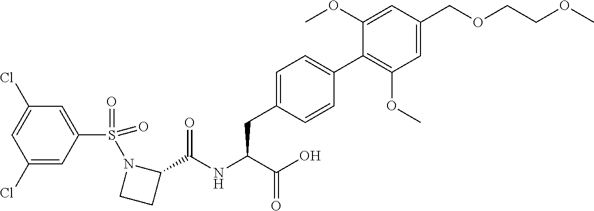 [SMILES: COCCOCc1cc(OC)c(-c2ccc(C[C@H](NC(=O)[C@@H]3CCN3S(=O)(=O)c3cc(Cl)cc(Cl)c3)C(=O)O)cc2)c(OC)c1] | 61% | ¹H NMR (400 MHz, DMSO- d6) δ ppm 12.97 (br. s, 1 H) 8.32 (d, J = 7.82 Hz, 1 H) 8.07 (t, J = 1.83 Hz, 1 H) 7.86 (d, J = 1.96 Hz, 2 H) 7.23 (d, J = 8.31 Hz, 2 H) 7.12 (d, J = 8.31 Hz, 2 H) 6.68 (s, 2 H) 4.54 (dd, J = 9.17, 7.21 Hz, 2 H) 4.50 (s, 2 H) 3.67- 3.77 (m, 2 H) 3.62 (s, 6 H) 3.56-3.61 (m, 2 H) 3.49-3.54 (m, 2 H) 3.28 (s, 3 H) 3.10 (m, J = 5.60 Hz, 1 H) 3.03 (m, J = 8.10 Hz, 1 H) 2.14-2.25 (m, | 703.1 705.1 (M + 23) |
| | | | | 1 H) 1.93-2.06 (m, 1 H) | |
| Cmpd 2087 | (S)-3-(4′-(2,5,8,11-tetraoxadodecyl)-2′,6′- dimethoxy-[1,1′-biphenyl]-4-yl)-2-((S)-1- (phenylsulfonyl)azetidine-2- carboxamido)propanoic acid | 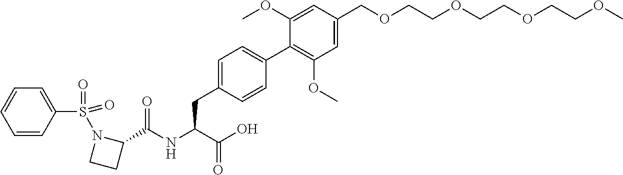 [SMILES: COCCOCCOCCOCc1cc(OC)c(-c2ccc(C[C@H](NC(=O)[C@@H]3CCN3S(=O)(=O)c3ccccc3)C(=O)O)cc2)c(OC)c1] | 85% | ¹H NMR (400 MHz, DMSO- d6) δ ppm 12.96 (br. s, 1 H) 8.18 (d, J = 8.07 Hz, 1 H) 7.81- 7.87 (m, 2 H) 7.74-7.81 (m, 1 H) 7.64-7.71 (m, 2 H) 7.24 (d, J = 8.07 Hz, 2 H) 7.12 (d, J = 8.07 Hz, 2 H) 6.68 (s, 2 H) 4.52- 4.57 (m, 1 H) 4.51 (s, 2 H) 4.30 (dd, J = 9.29, 7.34 Hz, 1 H) 3.67 (m, J = 4.60 Hz, 1 H) 3.62 (s, 6 H) 3.59 (s, 4 H) 3.48-3.57 (m, | 723.2 (M + 23) |
| | | | | 7 H) 3.38-3.44 (m, 2 H) 3.22 | |
| | | | | (s, 3 H) 3.14 (dd, J = 1.00 Hz, 1 | |
| | | | | H) 3.03 (dd, J = 7.80 Hz, 1 H) | |
| | | | | 1.99-2.12 (m, 1 H) 1.86-1.98 | |
| | | | | (m, 1 H) | |
| Cmpd 2088 | (S)-3-(4′-(2,5,8,11-tetraoxadodecyl)-2′,6′- dimethoxy-[1,1′-biphenyl]-4-yl)-2-((S)-1- ((3,5-dichlorophenyl)sulfonyl)azetidine-2- carboxamido)propanoic acid | 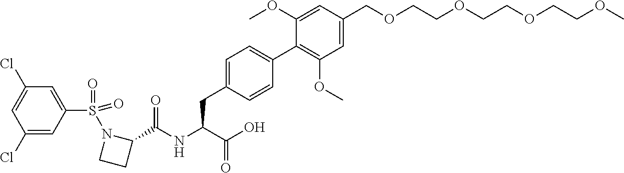 [SMILES: COCCOCCOCCOCc1cc(OC)c(-c2ccc(C[C@H](NC(=O)[C@@H]3CCN3S(=O)(=O)c3cc(Cl)cc(Cl)c3)C(=O)O)cc2)c(OC)c1] | 73% | ¹H NMR (400 MHz, DMSO- d6) δ ppm 12.96 (br. s, 1 H) 8.32 (d, J = 7.82 Hz, 1 H) 8.07 (t, J = 1.83 Hz, 1 H) 7.86 (d, J = 1.96 Hz, 2 H) 7.23 (d, J = 8.31 Hz, 2 H) 7.12 (d, J = 8.07 Hz, 2 H) 6.68 (s, 2 H) 4.52-4.58 (m, 2 H) 4.51 (s, 2 H) 3.65-3.80 (m, 2 H) 3.62 (s, 6 H) 3.59 (s, 4 H) 3.47-3.57 (m, 6 H) 3.39-3.44 | 791.1 793.1 (M + 23) |
| | | | | (m, 2 H) 3.22 (s, 3 H) 3.11 (dd, | |
| | | | | J = 5.10 Hz, 1 H) 3.03 (dd, | |
| | | | | J = 8.10 Hz, 1 H) 2.12-2.25 (m, | |
| | | | | 1 H) 1.92-2.07 (m, 1 H) | |
| Cmpd 1747 | 4-(((S)-2-(((S)-1-carboxy-2-(2′,6′- dimethoxy-4′-((2-methoxyethoxy)methyl)- [1,1′-biphenyl]-4- yl)ethyl)carbamoyl)pyrrolidin-1-yl) sulfonyl)benzoic acid | 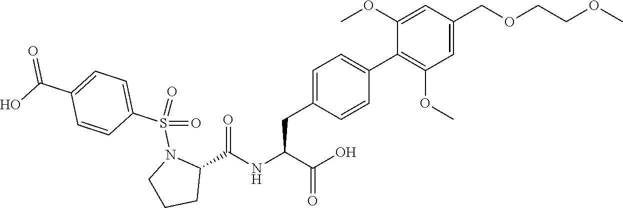 [SMILES: COCCOCc1cc(OC)c(-c2ccc(C[C@H](NC(=O)[C@@H]3CCCN3S(=O)(=O)c3ccc(C(=O)O)cc3)C(=O)O)cc2)c(OC)c1] | 62% | ¹H NMR (400 MHz, DMSO- d6) δ ppm 13.20 (br. s, 2 H) 8.19 (d, J = 8.07 Hz, 1 H) 8.03- 8.12 (m, 2 H) 7.86-7.91 (m, 2 H) 7.23 (d, J = 8.31 Hz, 2H) 7.11 (d, J = 8.07 Hz, 2 H) 6.67 (s, 2 H) 4.43-4.53 (m, 3 H) 4.18 (dd, J = 7.09, 3.91 Hz, 1 H) 3.61 (s, 6 H) 3.56-3.60 (m, 2 H) 3.47-3.53 (m, 2 H) 3.27 (s, 3 H) 3.07-3.22 (m, 3 H) 3.02 (m, J = 9.00 Hz, 1 H) 1.54-1.67 (m, 3 H) 1.40-1.52 (m, 1H) | 671.3 (M + H) |

VCAM-1 Assay Results

Flow Cytometry Cell-Based Assay.

Compounds were tested for their ability to inhibit the binding of soluble VCAM-1 to human G2 acute lymphoblastic leukemia (ALL) cells or 5TGMI myeloma cells. Briefly, G2 ALL or 5TGMI cells are pre-incubated with increasing concentrations (0.001 to 1000 nM) of compounds for 30 minutes. Soluble VCAM/Fc chimera protein (R&D systems) is then added to the mixture and the cells incubated for an additional 30 minutes. Afterwards, cells are washed and VCAM-1 is detected using PE-conjugated secondary mAbs. In each experiment, an aliquot of cells will be stained with isotype control mAbs to serve as a negative control. The percentage of VCAM-1 binding cells was then determined by flow cytometry.

TABLE 10
Inhibition of sVCAM-1 binding to human G2 ALL cells.

| Example | sVCAM-1 IC₅₀ (nM) |
|---|---|
| Firategrast | 230 |
| 822 | 69 |
| 823 | 0.5 |
| 824 | 0.6 |
| 825 | 48 |
| 842 | 0.4 |
| 1219 | 72 |
| 1220 | 0.47 |
| 1221 | 0.58 |
| 1222 | 218 |
| 1223 | 70 |
| 1224 | 1.57 |
| 1554 | 1.47 |
| 1555 | 12.9 |
| 1570 | 2.97 |
| 1571 | 4.43 |
| 1608 | 2.47 |
| 1609 | 11.7 |
| 1610 | 0.83 |
| 1611 | 0.49 |
| 1632 | 10 |
| 1633 | 3.33 |
| 1745 | 3.70 |
| 1746 | 3.79 |
| 1747 | 7.58 |
| 2087 | 2.06 |
| 2088 | 0.70 |
| 009 | 4.47 |

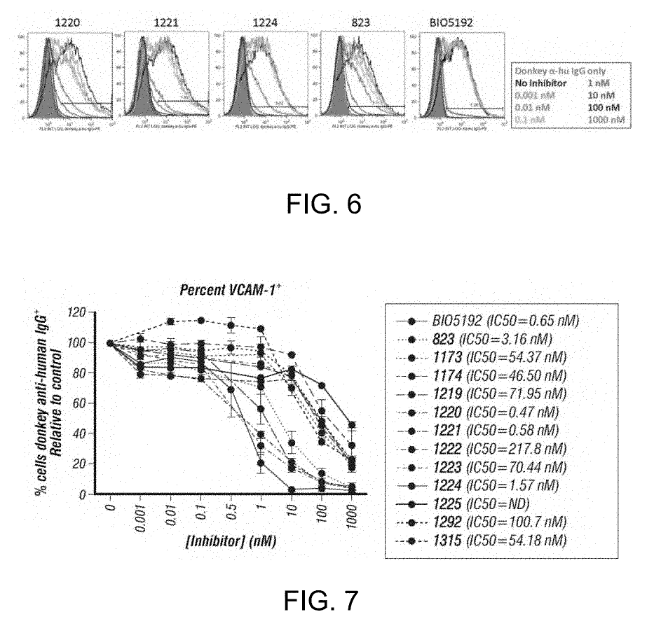

Treatment of cells with one subset of the inhibitors was interrogated by flow sorting and fluorescence analysis as is seen in FIG. 5. Treatment with Firategrast or BIO5192 were used as controls. With increasing concentrations of inhibitors, the fluorescence peak shifts left, corresponding to a decrease in fluorescence and a decrease in VCAM-1 binding. These data are also presented as percentage of VCAM-1 binding positive cells relative to the vehicle control. Several inhibitors had similar activity to Firategrast (Compounds 822 and 825), while others were significantly more potent (823, 824, and 842, and BIO5192). Other compounds were examined for greater potency than 823, again using flow cytometric analysis (FIG. 6). While BIO5192 was still more potent than the experimental inhibitors, 1220, 1221, and 1224 were all found to be more potent inhibitors than 823 in the sVCAM-1 assay (FIG. 6). The percentage of VCAM-1 positive cells relative to the control was also calculated for a variety of compounds, as seen in FIG. 7, further demonstrating that 1220, 1221, and 1224 exhibit greater potency than 823.

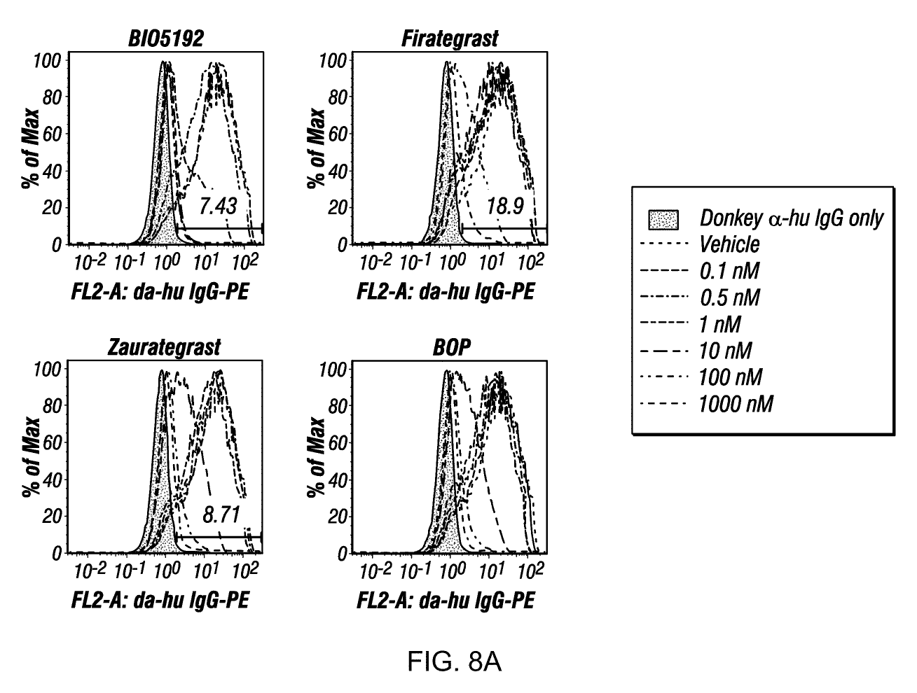

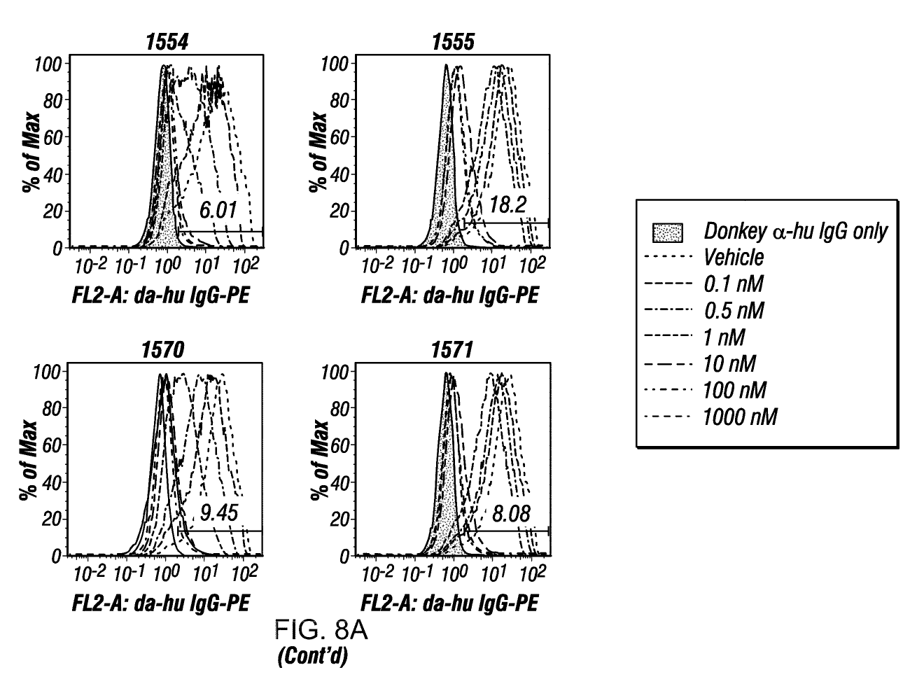

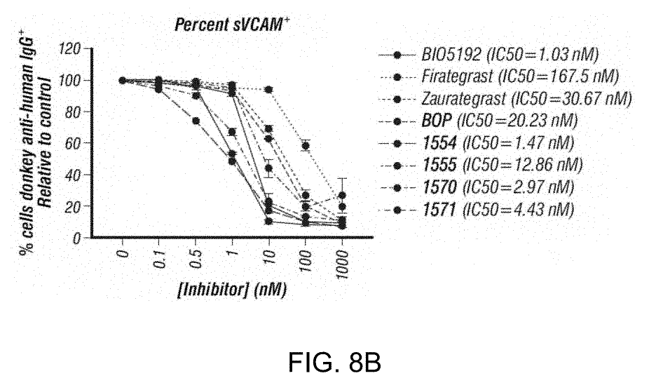

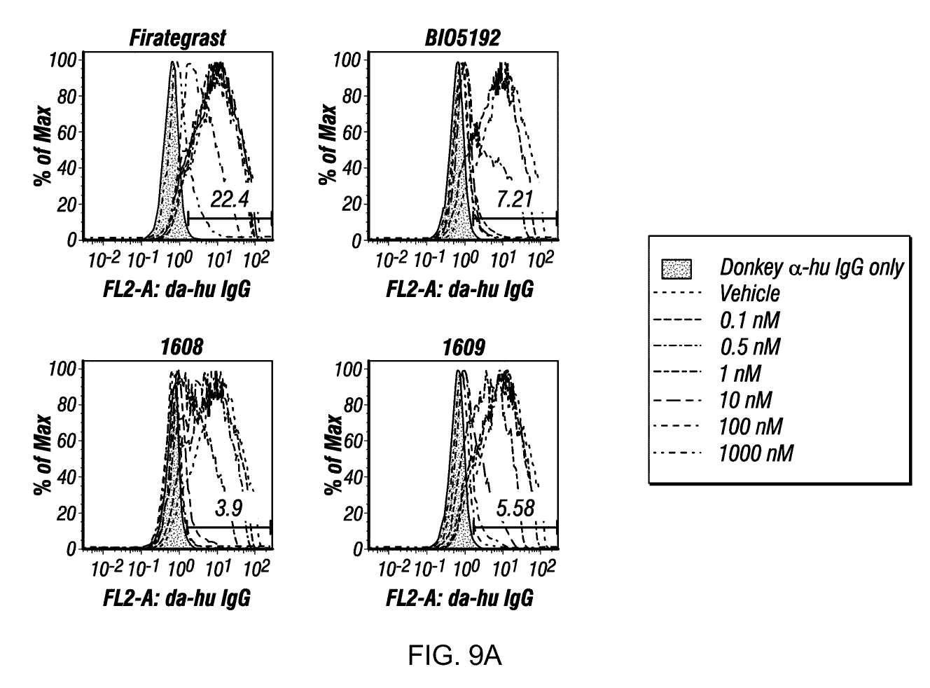

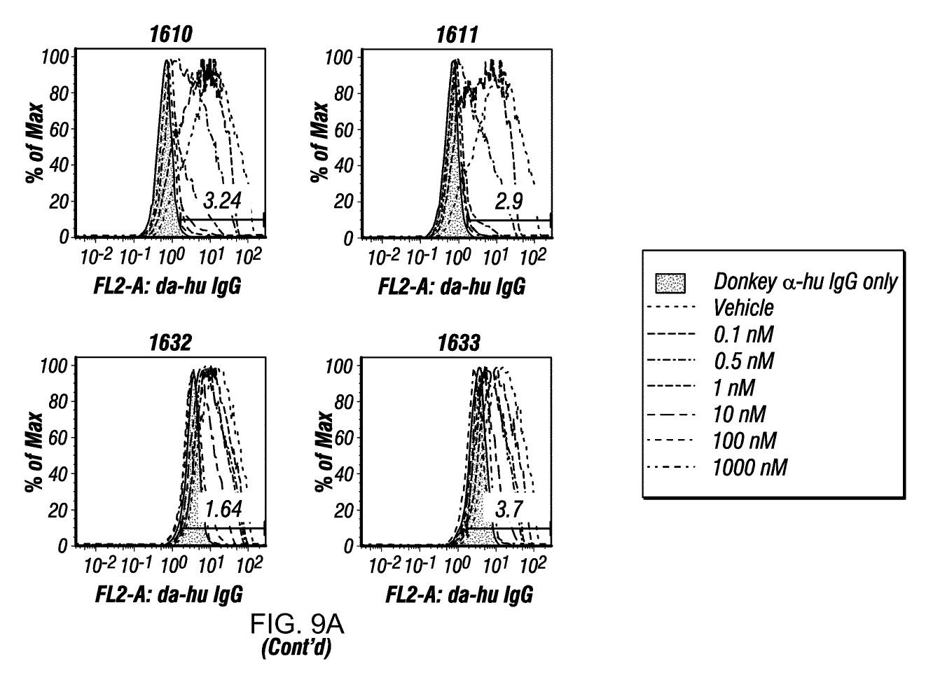

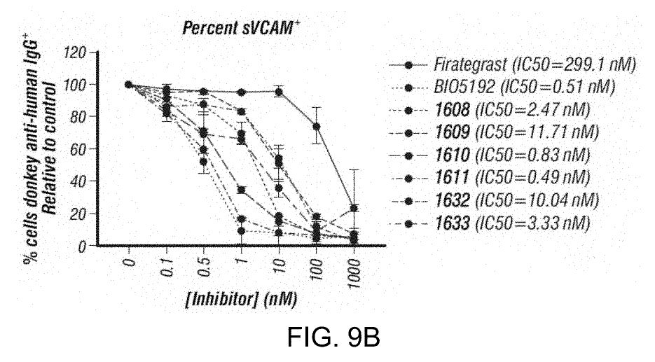

Further flow cytometric analyses were performed as seen in FIG. 8A, including treatment with Zaurategrast, another known integrin antagonist. The percentage of sVCAM positive cells, relative to the control after treatment with each inhibitor, was calculated from the flow cytometry analysis, and each of the inhibitors (1554, 1555, 1570, and 1571) exhibited a greater inhibition of sVCAM-1 binding than did Firategrast (FIG. 8B). The potency of compounds 1608, 1609, 1610, 1611, 1632, and 1633 in an sVCAM-1 assay was examined by flow cytometry, as seen in FIG. 9A. The percentage of VCAM-1 positive cells was calculated relative to the control as well (FIG. 9B). Each of the compounds was found to be significantly more potent than Firategrast.

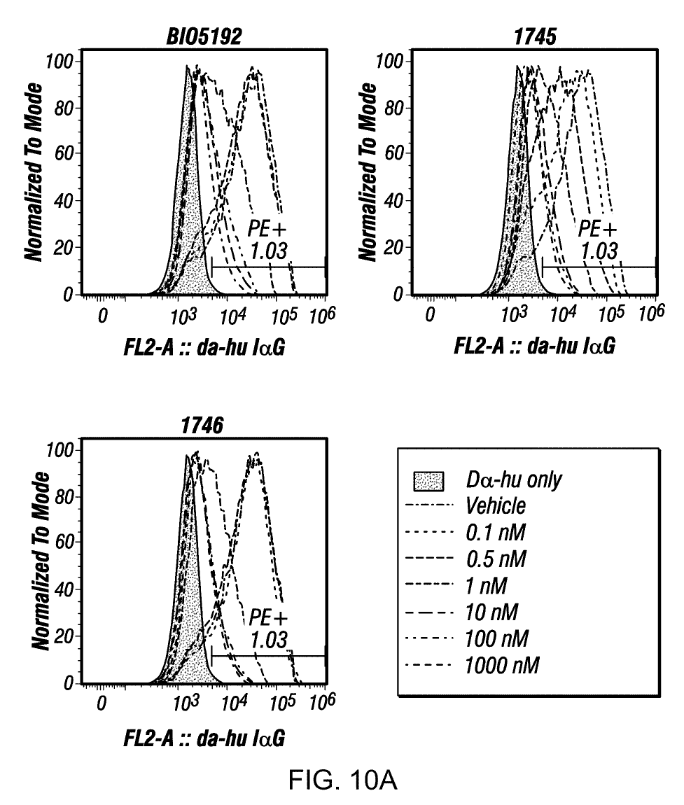

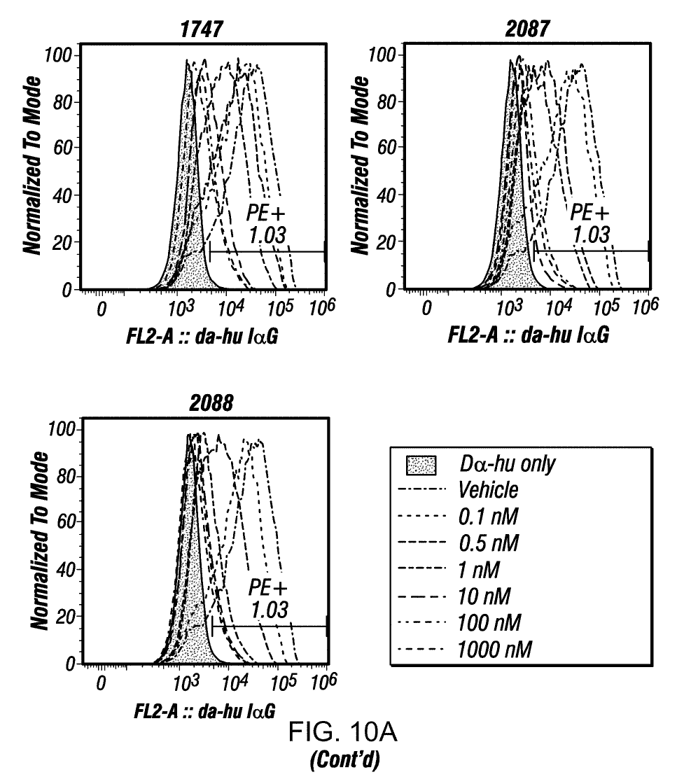

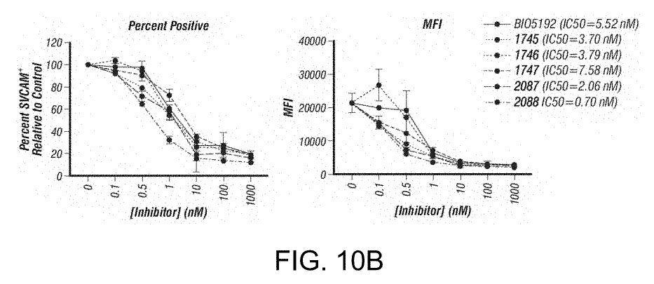

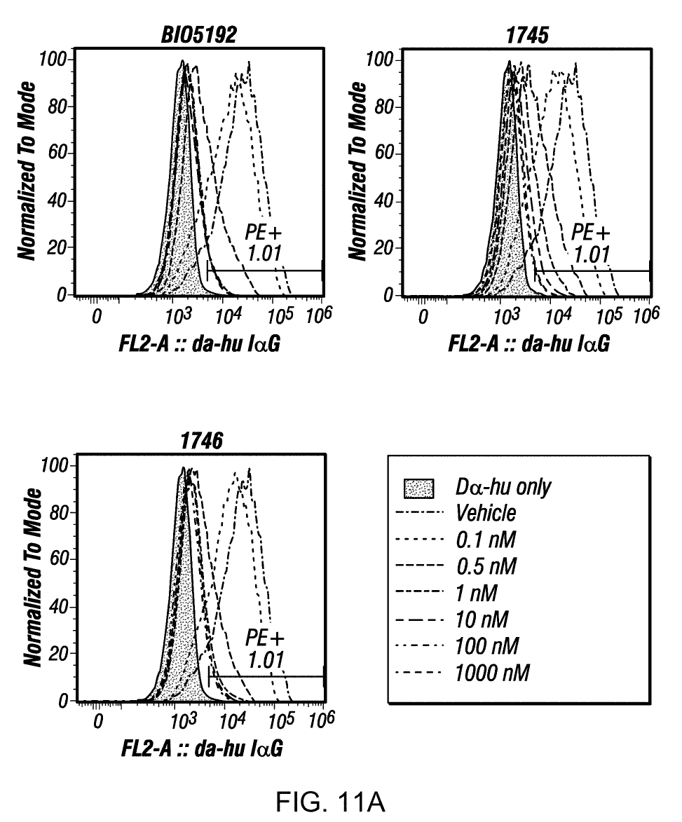

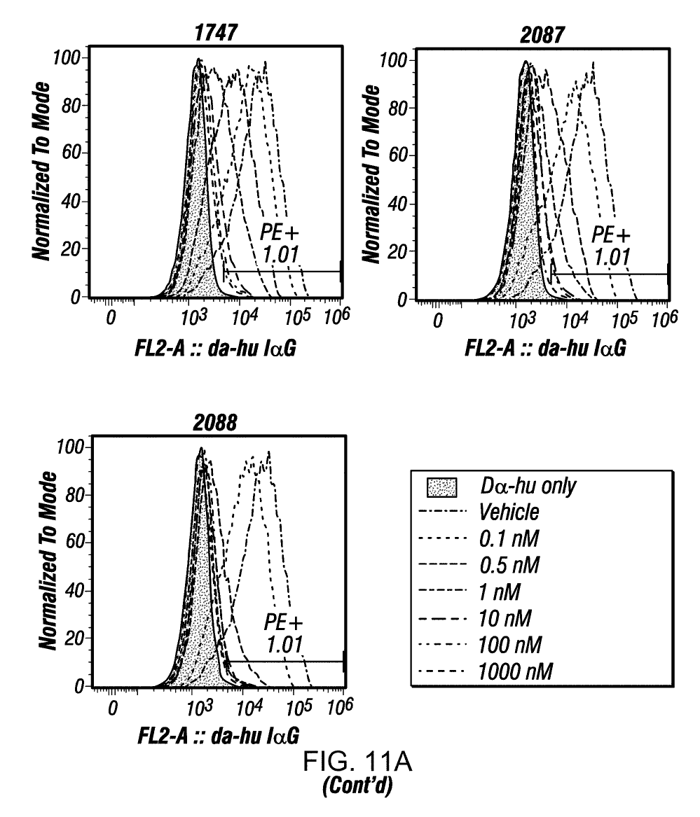

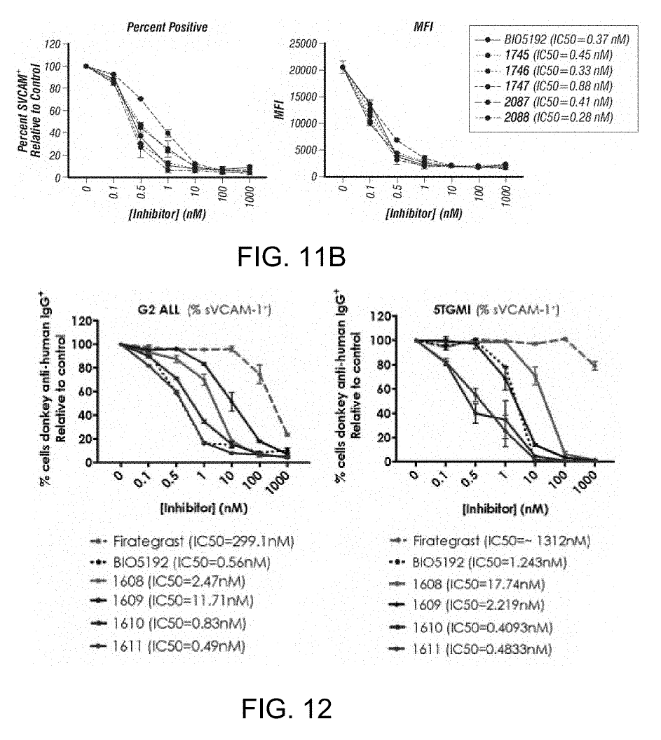

Treatment of cells with yet another set of inhibitors is shown over two experiments in FIGS. 10A & 11A. As can be seen in both experiments, with increasing concentrations of inhibitors, the fluorescence peak shifts left, corresponding to a decrease in fluorescence and a indicating a decrease in VCAM binding. The median fluorescence intensity for the cell lines was calculated after treatment with a subset of the inhibitors (1745, 1746, 1747, 2087, and 2087) at the listed concentrations (FIGS. 10B & 11B). The percentage of sVCAM positive cells was further calculated relative to the control for the selected inhibitors, as seen in FIGS. 10B & 11B. The inhibitor compounds all caused a very similar decrease in percentage of sVCAM positive cells with increasing concentrations of inhibitors, though inhibition was greatest with 2088, 1746, and the BIO5192 control.

Several inhibitors were tested on multiple cell types. Compounds 1608-1611 were evaluated for in sVCAM-1 assays on both G2 cells and 5TGMI myeloma cells. 5TGMI cells are noticeably more resistant to treatment with Firategrast than the G2 cells (FIG. 12). Each of compounds 1608-1611 were significantly more potent inhibitors of VCAM binding than Firategrast in both cell types, while compounds 1610 and 1611 are even more potent than BIO5192 in the 5TGMI cells (FIG. 12).

CFC Mobilization by Combination Therapies

Mice.

DBA/2J, C57BL/6J (CD45.2) and syngeneic B6.SJL-Ptprc^{a} Pep3^{b}/BoyJ (CD45.1) mice were purchased from the Jackson Laboratory (Bar Harbor, Me., USA). F1-hybrid mice (CD45.1/2) were obtained through breeding CD45.2 and CD45.1 mice. Animals were housed at the Washington University Medical School vivarium under SPF conditions. All experiments were performed in accordance with the guidelines of the Washington University Animal Studies Committee and the institutional animal care and use committee (IACUC), in agreement with AAALAC guidelines. Following lethal irradiation (1×9.5 or 11.0 Gy, using a ¹³⁷Cesium source) and transplantation, mice were kept on antibiotic medication, sulfamethoxazole and trimethoprim, 0.5 and 0.1 mg/mL respectively, (Hi-Tech Pharmacal, Amityville, N.Y., USA) peroral in drinking water.

Hspc Mobilization.

Recombinant human CXCL2 (Groβt; R&D systems) was reconstituted in sterile Ca⁺²/Mg⁺²-free phosphate buffered saline (PBS) and injected subcutaneously (SC) at a dose of 2.5 mg/kg. Firategrast (U.S. Patent Application No. 2014/051655) was dissolved in Ca⁺²/Mg⁺²-free PBS plus 1% ethanol and injected intravenously at a dose of 100 mg/kg. RhG-CSF (Neupogen®, Filgrastim, Amgen, Thousand Oaks, Calif., USA) diluted in PBS was injected i.p. every 12 hrs at a dose of 100 μg/kg for a total of 9 (day 5) doses.

Colony Forming Cell (CFC) Assay.

Peripheral blood (PB) was drawn from the facial vein without anaesthesia into K/EDTA anti-coagulated tubes (Sarstedt AG & Co, Nimbrecht, Germany). Red blood cells were removed from 25 μL aliquots of blood using hypotonic lysis (Ammonium-Chloride-Potassium, ACK buffer, 5-10 min at RT) and samples were mixed with 2.5 mL methylcellulose media supplemented with a cocktail of recombinant cytokines (MethoCult 3434; Stem Cell Technologies, Vancouver, BC, Canada). Cultures were plated in duplicate in 35 mm dishes and placed in a humidified chamber with 5% CO₂ at 37° C. After 7 d of culture, colonies containing at least 50 cells were counted using an inverted microscope in a blinded fashion.

Flow Cytometric Enumeration of Murine HSPCs.

Peripheral blood (PB) was drawn from the facial vein without anaesthesia into K/EDTA anti-coagulated tubes (Sarstedt AG & Co, Nimbrecht, Germany). Cell counts (WBC) were determined using an automatic hemocytometer (Hemavet 950, Drew Scientific, Dallas, Tex.). Red blood cells were removed from 25 μL aliquots of blood using hypotonic lysis (Ammonium-Chloride-Potassium, ACK buffer, 5-10 min at RT). Samples were resuspended in staining buffer (PBS supplemented with 0.5% bovine serum albumin and 2 mM EDTA) and incubated for 30 min at 4° C. with pre-titrated saturating dilutions of the following fluorochrome-labeled monoclonal antibodies (BD Biosciences, San Jose, Calif.; clone designated in parenthesis): Sca1-PE (D7), CD117-BV421 (ACK2), lineage-APC (17A2, RB6-8C5, RA3-6B2, Ter-119, M/170) and CD45-A700 (30-F11). Dead cells were excluded from these assays by staining with 2 μg/mL 7-amino-actinomycin D (Molecular Probes, Eugene, Oreg.) for 5 min prior to analysis. Appropriate isotype-matched negative controls were used to assess background fluorescence intensity. Samples were analyzed on a Beckman Coulter Gallios flow cytometer and data were analyzed using FlowJo software (TreeStar, Ashland, Oreg.). Absolute numbers of immunophenotypically defined populations (e.g. LSK) were calculated by identifying CD45 positive cells as WBC from the corresponding cell count analysis. Statistical comparisons of flow cytometry data were performed using an unpaired two-tailed Student t-test (GraphPad Prism). P-values ≤0.05 were considered significant.

Repopulating Unit (RU) Assay.

An RU assay was performed to directly compare the repopulating capacity of peripheral blood mobilized with different combinations of compounds. Lethally irradiated CD45.2 hosts received transplants consisting of 6 μL of blood from mobilized mice (CD45.1+) together with 2.5×10⁵ CD45.2+BM competitor cells. After 20 weeks, blood graft-derived RUs were calculated for the recipient mice by the following formula: RU=(D×C)/(100−D) where D is the percentage of blood-derived B lymphocytes and myeloid cells and C is the number of RUs contained in the competitor BM fraction of the graft (C=2.5 for 250,000 competitor BM cells).

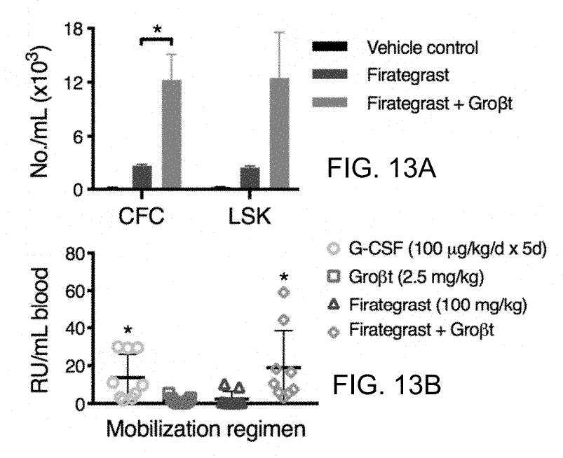

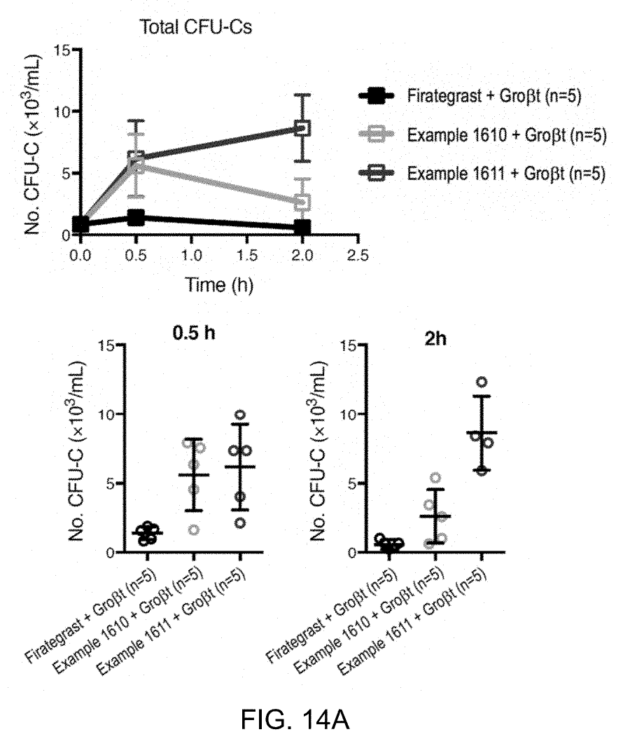

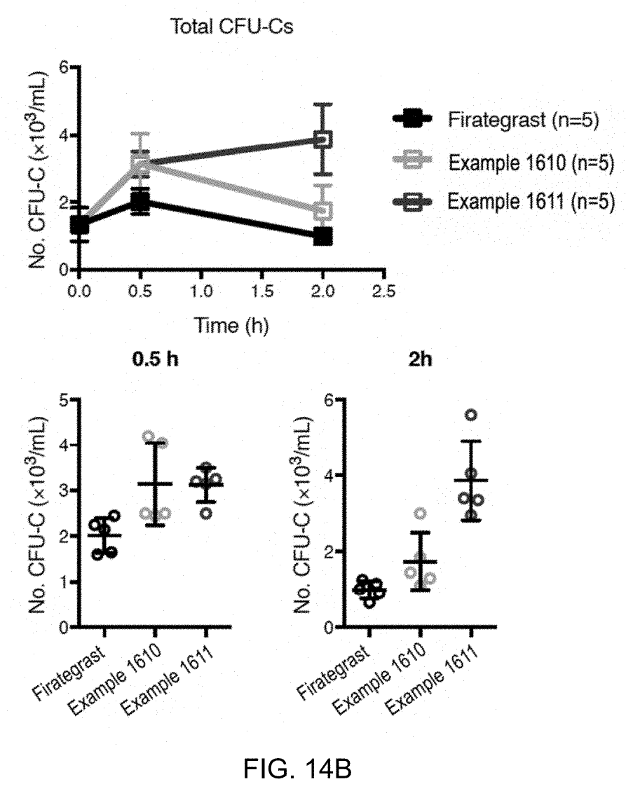

The results of these test are shown in FIG. 13 and FIG. 14.

Additional Studies of Combination of VLA-4 Inhibitors

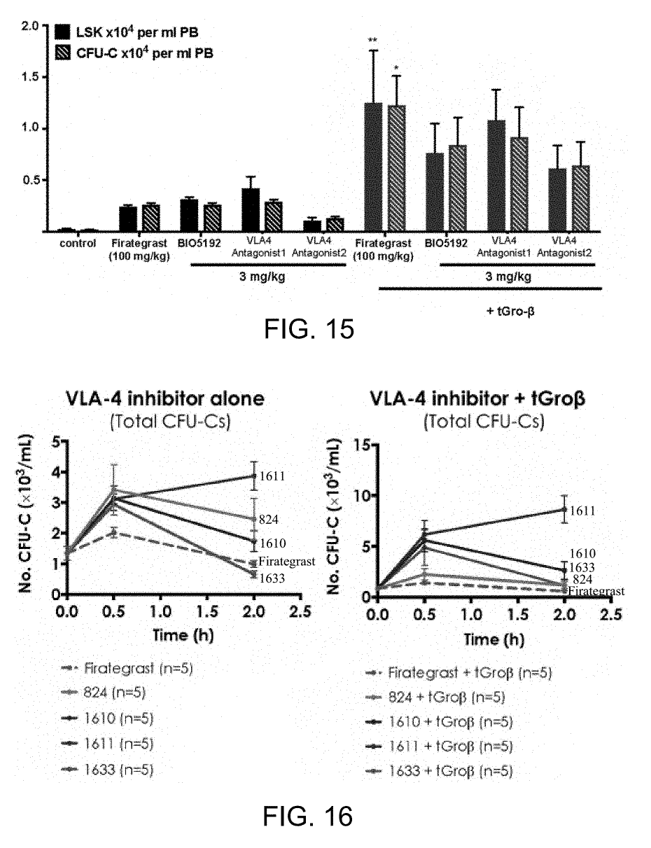

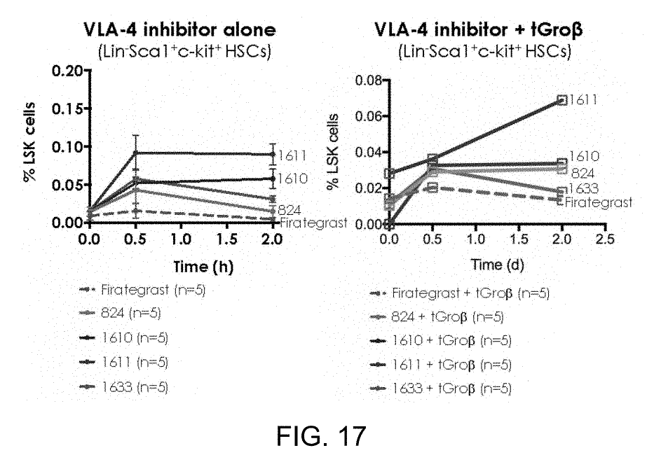

Using the combination of VLA4 blockade (using firategrast, BIO5192, 823 (VLA4 Antagonist 1), and 842 (VLA4 Antagonist 2) with CXCR2 stimulation, both CFU-C and LSK cells were mobilized. See FIG. 15. Another subset of inhibitors (824, 1610, 1611, and 1633) was tested for CFU-C mobilization in DBA/2J mice with VLA-4 inhibitors and Groβt (FIG. 16). Treatment with compounds 35, 36, and 12 nearly doubled the amount of CFU-C cells mobilized when treated in combination with Groβt, than with just inhibitor alone (FIG. 16). Compounds 824, 1610, 1611, and 1633 were also found to mobilize LSK cells (FIG. 17) at a greater extent than firategrast.

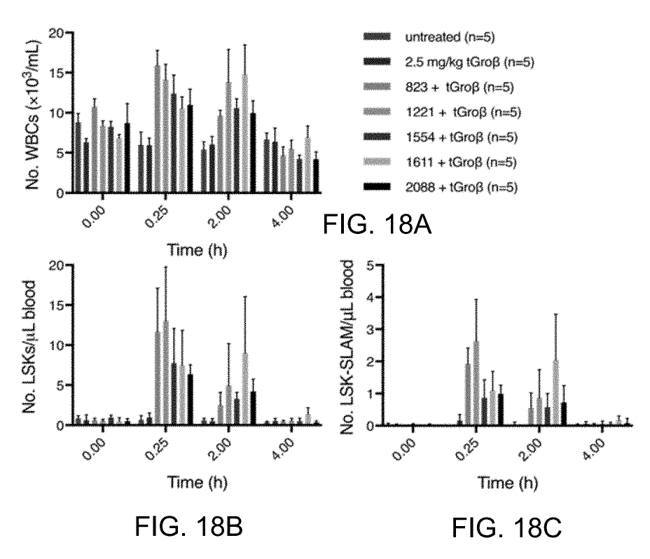

Further studies were carried out to determine the effectiveness of treatment with other VLA-4 inhibitors and Groβt in mobilizing stem cells. Mice injected with the VLA-4 inhibitor and Groβt had peripheral blood collected at 0.25, 2, and 4 hours post injection. The blood aliquots were analyzed by flow cytometry to separate LSK cells and LSK-SLAM cells and further analyzed by Hemavet. Hemavet analysis of total WBCs shows significantly greater mobilization of stem cells after 0.25 or 2 hours in mice treated with both VLA-4 inhibitors and Groβt than untreated cells, or cells treated only with Groβt. See FIG. 18A. The total WBCs returned to levels similar to baseline after 4 hours. As can be seen in FIGS. 18B & 18C, both LSK cells and LSK-SLAM cells were significantly mobilized by treatment with the combination of VLA-4 inhibitor and Groβt.

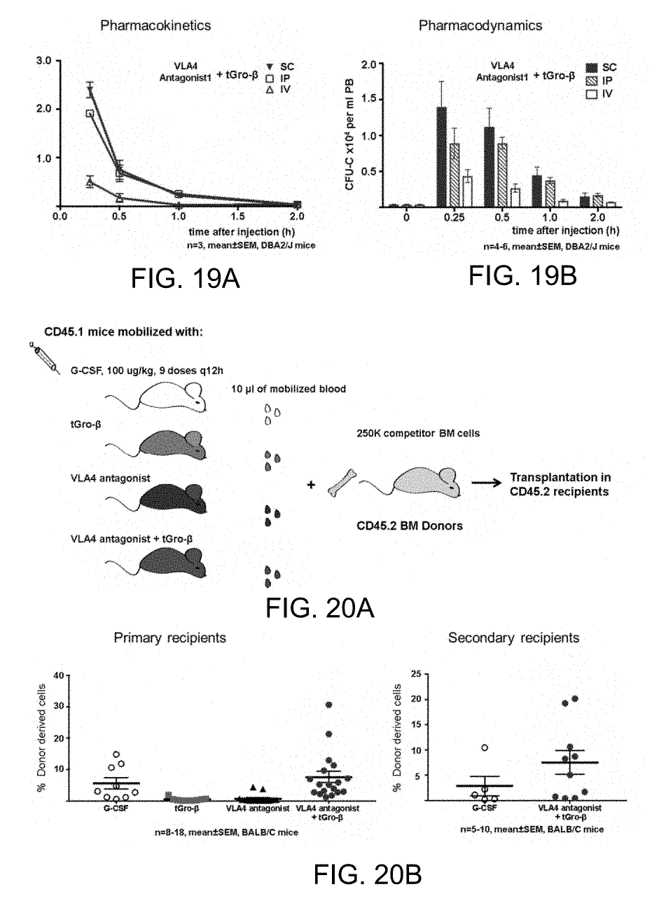

Additionally, the pharmacokinetics and pharmacodynamics of these combinations result in the fast mobilization of CFU-C per mL of peripheral blood. Three routes of administration were tested: subcutaneous, intraperitoneal, and intravenous administration. Subcutaneous and intraperitoneal showed higher CFU-C per mL of peripheral blood relative to intravenous administration and all routes of administration showed fast mobilization with the peak happening within 30 minutes of administration in DBA2/J mice. See FIGS. 19A & 19B.

As can be seen in FIGS. 20A & 20B, the repopulation unit assay shows that the mobilization methods described herein using a combination of a VLA4 antagonist (823) and Groβt result in mobilized stem cells which can be successfully transplanted in both primary and secondary recipients.

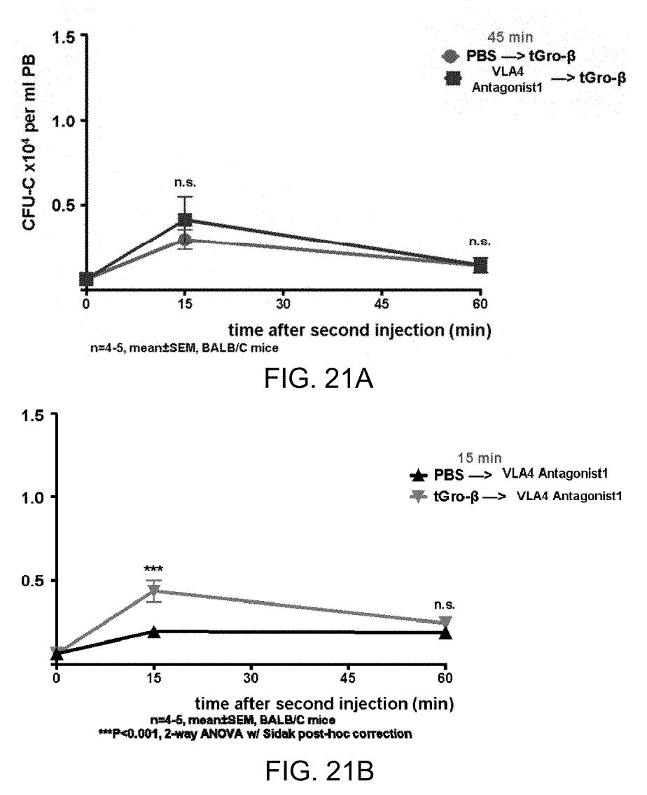

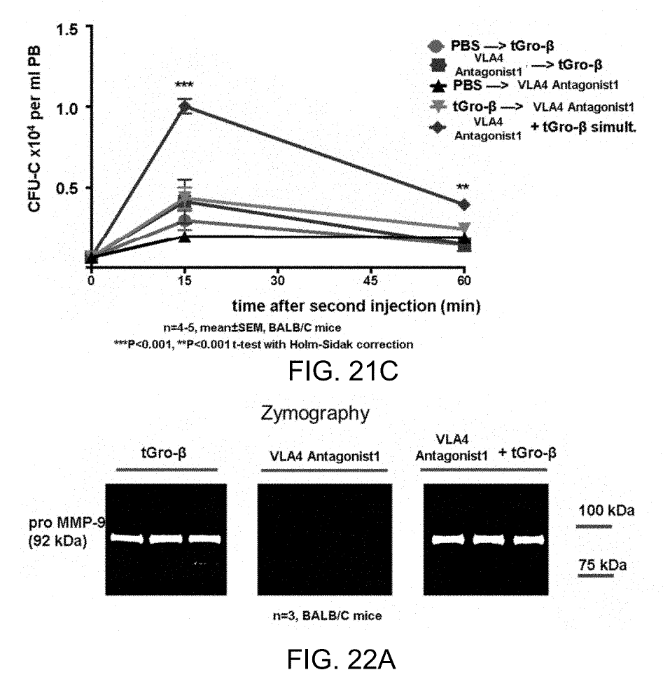

To determine if the order of administration matters, examples were the order of administration was varied. As can be seen in FIGS. 21A-21C, the highest number of colony forming units were obtained when the two agents were administered simultaneously. Without wishing to be bound by any theory, it is believed that CXCR2 stimulation primes the biological system for VLA4 blockage.

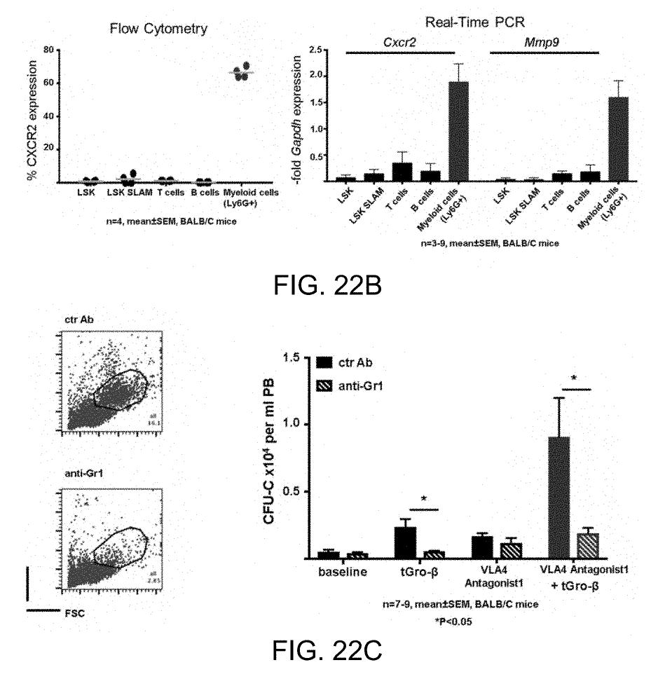

In order to determine the specific mechanism of action, studies were carried out to determine if MMP9 proteolytic activity was present in the compositions. In FIG. 22A, compositions which contained Groβt showed MMP9 activity. Using both flow cytometry and real-time PCR it was determined that myeloid cells express both MMP9 and CXCR2 and may play a role in the mobilization of the desired stem cells. See FIG. 22B. In an alternative example where GR1+ myeloid cells were removed through the use of a GR1+ antibody, the mobilization of CFU-C was greatly decreased relative to a control antibodies (FIG. 22C). Without wishing to be bound by any theory, it is believed that GR1+ myeloid cells play a role in stem cell mobilization.

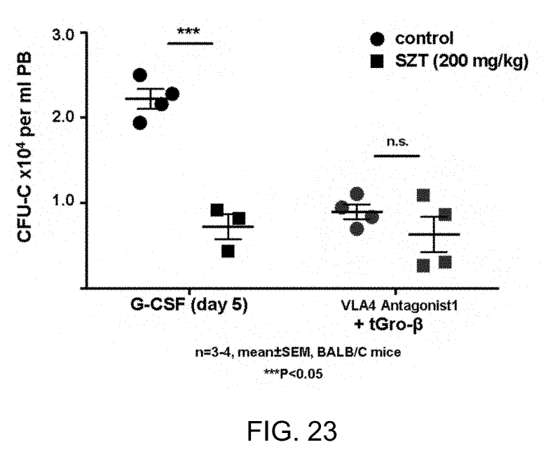

In order to determine the effectiveness of the combination in conditions which are typically difficult to achieve mobilization such as diabetes, a streptozotocin induced diabetes model was used. The combination of a VLA-4 inhibitor and Groβt resulted in similar mobilization regardless of the nature of the model animal (FIG. 23). Thus, the combinations described herein may be especially useful in conditions which are difficult to mobilize hematopoietic stem or progenitor cells.

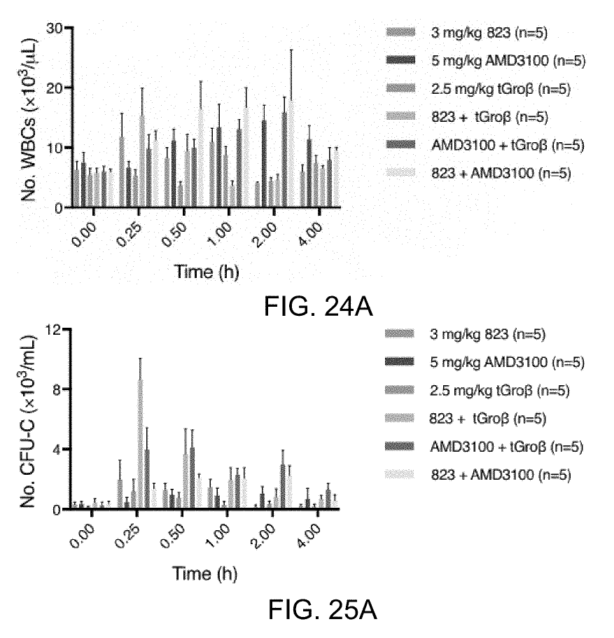

Further combinations of VLA-4 inhibitors with CXCR4 inhibitors and CXCR2 agonists were tested. Peripheral blood was taken from mice injected with either 823 (VLA-4 inhibitor) or AMD3100 (CXCR4 inhibitor), Groβt (CXCR2 agonist), or a combination of two of the aforementioned at baseline, 0.25, 0.5, 1, 2, and 4 hours post-injection. These blood aliquots were used for CFU assays and Hemavet analysis. WBCs were counted by Hemavet and significantly more stem cells were mobilized following treatment with the VLA-4 inhibitor and any of the listed combination therapies than with CXCR2 agonist alone or the CXCR4 inhibitor alone (FIG. 24A). As expected, there was a spike in CFUs for the treatment with just the VLA-4 inhibitor and combined VLA-4 inhibitor and CXCR2 agonist treatment, but there was also an increase in CFUs following the other combination treatments (FIG. 24B).

### Example 3 (Section III)

Methods and Materials—In Vitro Assays of HSPC Mobilization.

Colony Assays.

In short-term colony-forming cell (CFC) assays, test samples are cultured in a semi-solid matrix supplemented with nutrients and cytokines for −1 week at 37° C. During this culture period, CFC proliferate and produce discrete cell clusters or colonies of morphologically recognizable daughter cells that can be quantified by light microscopy. Here, mice will be treated with mobilization regimens and peripheral blood will be collected via the retroorbital sinus at the peak of mobilization. After collection of blood, a complete blood count will be performed using a Hemavet 950FS cell counter (Drew Scientific) and CFC assays will be established in methylcellulose-containing Iscove modified Dulbecco medium supplemented with interleukin-3, interleukin-6, and stem cell factor (MethoCult3534; StemCell Technologies). CPU-GM, BFU-E, and CFU-GEMM colony types will be enumerated following 1 week of culture at 37° C.

Flow Cytometric Enumeration of Murine HSPCs.

Among the subsets that define HSPCs, CD34-c-kit+Sca-1+lineage marker-(CD34-KSL) cells are regarded as one of the populations that have the highest enrichment of HSPCs in adult mouse bone marrow. More recently, the SLAM family markers, CD150 and CD48, have been used to differentiate stem cells from more committed progenitor cells. Here, primitive murine HSPC mobilization will be examined by flow cytometry enumeration of CD34-c-kit+Sca-1+lineage marker-CD150+CD48-cell numbers following treatment with mobilization regimens.

In Vivo Assays of HSPC Mobilization.

Competitive Repopulation Assays.

The definitive assay for stem cell activity in a test sample is the complete and sustained (>5 months) reconstitution of all hematopoietic lineages in irradiated recipients by transplanted HSPCs. For competitive stem cell repopulation assays, peripheral blood mononuclear cells (PBMCs) from wild-type C57BL/6 (CD45.2+) mice mobilized by the regimens described herein will be mixed with 2×105 competitor bone marrow cells from C57BL/6J×B6.SJL-Ptprca Pepcb/BoyJ F1 (CD45.1+/CD45.2+) mice and transplanted into lethally irradiated (1100 cGy) recipients congenic at the CD45.1+ locus (B6.SJL-Ptprca Pepcb/BoyJ). At least 3 mobilized PBMC:competitor ratios (typically the number of PBMCs isolated from 0.5, 1, and 1.5 mL of blood) will be evaluated to establish the appropriate donor:competitor ratio. Secondary transplantations will be performed by intravenously injecting 106 unfractionated bone marrow cells from donors 5 months after transplantation into lethally irradiated (1100 cGy) CD45.1+ secondary recipients.

In Vitro Assay of Chemosensitization.

Stromal Protections Assay.

The impact of bone marrow stroma on leukemia cell survival during treatments listed herein will be assessed. Mouse AML (APL) and human ALL (G2) cell lines will be incubated with the appropriate drugs and drug combinations ±chemotherapy ±stroma. BLI (bioluminescence) as well as standard MTT assays will be used to assess cell growth and survival.

In Vivo Assay of Chemosensitization.

Recipient mice (C57BL/6) will be injected with 1×106 murine acute promyelocytic (APL) leukemia cells transduced with Click Beetle Red-GFPe. In the absence of treatment all mice develop overt leukemia by day +20 and die from a rapidly fatal leukemia with leukocytosis and splenomegaly by day +35. The treatments disclosed herein will be administered with and without chemotherapy and mouse survival and leukemia burden as measured by BLI and FACS (Gr-1+/c-kit+/CD34+ leukemia cells) will be assessed. Both BLI and FACS measurements will be obtained on days +7, +14, +21, +28, and +35.

C57BL/6 into BALB/c Transplant Model.

Lethal Irradiation of BALB/c Recipients.

Recipient BALB/c mice are lethally irradiated with 925 cGy of total body irradiation on day −1 and then infused with 5×106 T cell depleted bone marrow cells (TCD-BM) containing 2×106 T cells from C57BL/6 mice on day 0. Mice are monitored for indications of GvHD (clinical score based on weight loss, hunched posture, activity, fur texture, diarrhea and skin integrity) and survival time. Signs of GvHD are typically seen within 2-3 weeks and all mice die by 4-5 weeks. The rate of GvHD in irradiated mice receiving TCD-BM only (n=15 mice) will be compared with the rate in mice receiving TCD-BM plus the different donor PBMC populations described herein. Mice will be sacrificed on day 25 to collect and examine GvHD target organs (histology of liver, intestines, and spleen). Peripheral blood will also be collected on day 25 to determine the extent of donor cell engraftment and immune reconstitution (flow cytometry using antibodies against CD3, B220, CD4, CD8, FOXP3, H-2Kb, CD45.1, and CD45.2).

Sublethal Irradiation of BALB/c Recipients.

Recipient BALB/c mice are sublethally irradiated with 500 cGy of total body irradiation on day and then infused with 5×106 T cell depleted bone marrow cells (TCD-BM) containing 2×106 T cells from C57BL/6 mice on day 0. Mice are monitored for indications of GvHD and donor engraftment as described above.

C57BL/6 into B6D2F1 Transplant Model.

Lethal Irradiation of B6D2F1 Recipients.

Recipient B6D2F1 mice are lethally irradiated with 1100 cGy (split doses, 3 hr apart) of total body irradiation on day −1 and then infused with 1 ×10 T cell depleted splenocytes (TCD-SPL) containing 3×106 T cells from C57BL/6 mice on day 0. The rate of GvHD and donor engraftment in irradiated mice receiving TCD-SPL only (n=15 mice) will be compared with the rate in mice receiving TCD-SPL plus the different purified T cell populations listed herein. Mice will be monitored for indications of GvHD and donor engraftment as described above.

VLA-4 Antagonists Enhance Mobilization of HSPCs.

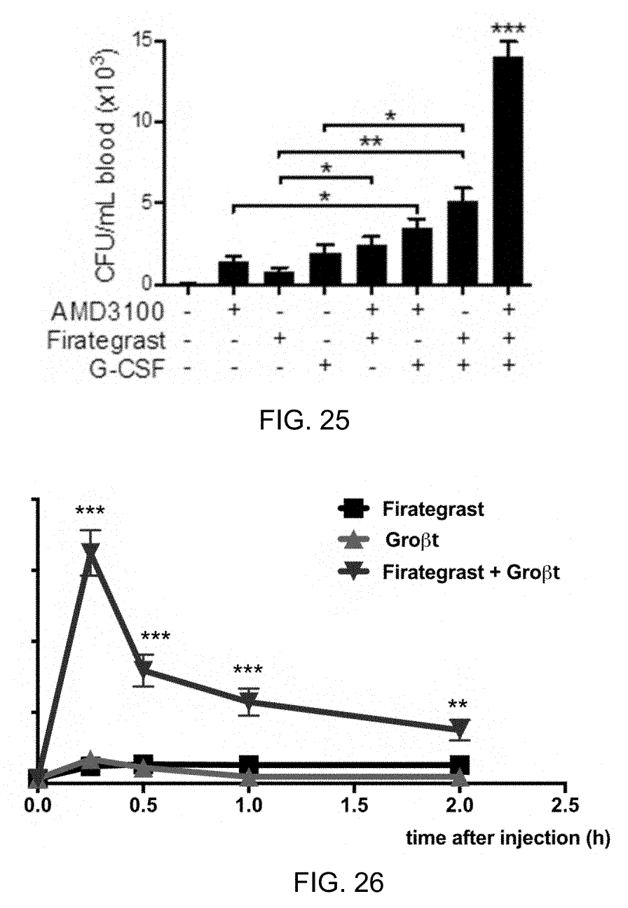

Treatment of mice with the VLA-4 antagonist BIO5192 results in a 30-fold increase in mobilization of HSPCs and combination of BIO5192 with plerixafor gave a further 3-fold increase. Treatment with a combination of BIO5192, plerixafor, and G-CSF enhanced mobilization by 17-fold compared to G-CSF alone. BIO5192 also mobilized long-term repopulating cells that successfully engraft and expand in a multi-lineage manner in secondary transplant experiments. Similar mobilization studies were performed using the small molecule VLA-4 antagonist firategrast. Similar results relative to BIO5192 were obtained (FIG. 25). Although significant additive/synergistic effects were observed following combination therapy with firategrast and AMD3100 and/or G-CSF, 30-fold higher concentrations of firategrast were required to mimic the mobilization effects of BIO5192 (100 mg/kg vs. 3 mg/kg, respectively). This decreased biological activity is presumably due to firategrast's ˜30-fold lower potency for VLA-4.

A combination under investigation uses firategrast with the CXCR2 agonist truncated Gro-beta (Gro-βt, SB-251353). Gro-βt is a recombinant, N-terminal truncated form of Gro-β that binds to CXCR2 with greater potency than the full-length form of Gro-β. If both firategrast and Gro-βt are given to mice within 5 minutes of each other and blood is collected 15 min post-administration, a significant synergy in colony-forming cell (CFC) mobilization is observed that persisted for >2 hours (FIG. 26). Remarkably, the level of CFC mobilization achieved with the firategrast and Gro-βt regimen is greater than that of G-CSF used alone for 4 days.

Combination of Groβ or Truncated Groβ (Groβt) Plus a VLA-4 Small Molecule Inhibitor for Hematopoietic Stem Cell Mobilization and Leukemia Chemosensitization. Mobilization of HSPCs with Gro-βt and Firategrast

This experiment showed that the combination of Gro-βt (2.5 mg/kg, SC) and firategrast (VLA-4 inhibitor; 100 mg/kg, IV) synergistically mobilized murine HSPCs, as measured by a colony-forming unit (CFU) assay of mobilized peripheral blood. The combination of Gro-βt and firategrast mobilized 5.6-fold more murine CFUs (14,210±843 CFU/mL blood) compared to when each agent was administered alone (Gro-βt: 2,520±580 CFU/mL; firategrast: 2,480±230 CFU/mL) in DBA/2 mice. This experiment was carried out again and similar results were obtained. In the second experiment, the combination of Gro-βt (2.5 mg/kg, SC) and firategrast (100 mg/kg, IV) mobilized 9.5-fold more murine CFUs (16,225±3,600 CFUs) compared to when each agent was administered alone (Gro-βt: 1,710±600 CFU/mL; firategrast: 1,205±290 CFU/mL) in DBA/2 mice. To ensure the synergistic CFU mobilization that was observed with the combination of Gro-βt and firategrast in DBA/2 mice in the previous experiments, the administration of these two agents was also tested in a different mouse strain. Specifically, the combination of Gro-βt (2.5 mg/kg, SC) and firategrast (100 mg/kg, IV) mobilized 7.3-fold more murine CFUs (4,600±1,600 CFU/mL) compared to when each agent was administered alone (Gro-βt: 630±390 CFU/mL; firategrast: 430±130 CFU/mL) to C57BL/6 mice. Furthermore, the combination of Gro-βt and firategrast mobilized 18-fold more murine lineage-Sca-1+c-kit+(LSK) cells (72 LSK cells/μL blood), another measure of HSPCs, compared to when each agent was administered alone (Gro-βt: 4 LSK cells/μL; firategrast: 3 LSK cells/μL) to C57BL/6 mice.

Additional VLA-4 Antagonists—Preparation of Compounds

Table 11 below includes the characterization of the VLA-4 antagonist described herein

TABLE 11
Example Compounds and Characterization

| Ex. | | | | | LC/MS |
|---|---|---|---|---|---|
| No. | IUPAC Name | Structure | Yield | ¹H NMR δ | m/z |
| Cmpd 12 | (2S)-2-(2,6-dichlorobenzamido)- 3-(4′-(ethoxymethyl)-2-(1- hydroxyethyl)-2′,6′-dimethoxy- [1,1′-biphenyl]-4-yl)propanoic acid | 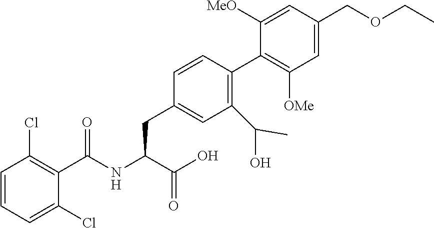 [SMILES: CCOCc1cc(OC)c(-c2ccc(C[C@H](NC(=O)c3c(Cl)cccc3Cl)C(=O)O)cc2C(C)O)c(OC)c1] | 78% | 12.80 (br. s., 1 H), 9.10 (t, J = 8.68 Hz, 1 H), 7.50 (dd, J = 7.09, 1.47 Hz, 1 H), 7.32- 7.46 (m, 3 H), 7.03-7.20 (m, 1 H), 6.78 (dd, J = 7.70, 5.75 Hz, 1 H), 6.67 (s, 2 H), 4.64- 4.83 (m, 2 H), 4.48 (s, 2 H), 4.35 (m, J = 6.40, 3.20, 3.20 Hz, 1 H), 3.63 (s, 6 H), 3.54 (q, J = 7.01 Hz, 2 H), 3.11- 3.24 (m, 1 H), 2.93 (dd, J = 14.06, 9.90 Hz, 1 H), 1.20 (t, J = 7.09 Hz, 3 H), 1.02 (dd, J = 7.70, 6.48 Hz, 3 H). | 598, 600 |
| Cmpd 21 | 4-{[(1S)-2-[4-(4-carbamoyl-2,6- dimethoxyphenyl)phenyl]-1- carboxyethyl]carbamoyl}-3,5- dichlorobenzoic acid | 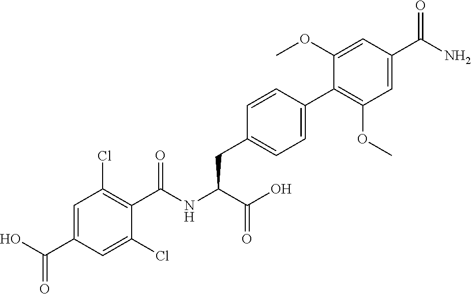 [SMILES: COc1cc(C(N)=O)cc(OC)c1-c1ccc(C[C@H](NC(=O)c2c(Cl)cc(C(=O)O)cc2Cl)C(=O)O)cc1] | 59% | 13.10 (br. s, 2 H) 9.20 (d, J = 8.31 Hz, 1 H) 7.83 (s, 2 H) 7.30 (d, J = 8.07 Hz, 2 H) 7.27 (s, 2 H) 7.13 (d, J = 8.07 Hz, 2 H) 4.72-4.82 (m, 1 H) 3.72 (s, 6 H) 3.33 (br. s, 2 H) 3.22 (dd, J = 13.94, 4.40 Hz, 1 H) 2.92 (dd, J = 14.06, 10.64 Hz, 1 H) | 562.0, 564.0 |
| Cmpd 22 | 4-{[(1S)-1-carboxy-2-{4-[4- (hydroxymethyl)-2,6- dimethoxyphenyl]phenyl}ethyl] carbamoyl}-3,5-dichlorobenzoic acid | 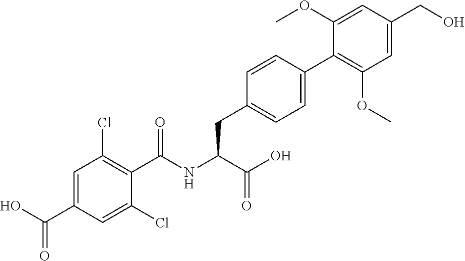 [SMILES: COc1cc(CO)cc(OC)c1-c1ccc(C[C@H](NC(=O)c2c(Cl)cc(C(=O)O)cc2Cl)C(=O)O)cc1] | 41% | 13.72 (br. s, 1 H) 12.90 (br. s, 1 H) 9.22 (d, J = 8.31 Hz, 1 H) 7.85 (s, 2 H) 7.26 (d, J = 8.07 Hz, 2 H) 7.08 (d, J = 8.07 Hz, 2 H) 6.69 (s, 2 H) 5.24 (br. s, 1 H) 4.66-4.82 (m, 1 H) 4.52 (s, 2 H) 3.65 (s, 6 H) 3.20 (dd, J = 14.18, 4.65 Hz, 1 H) 2.91 (dd, J = 14.31, 10.64 Hz, 1 H) | 548.0, 550.0 |
| Cmpd 23 | 4-{[(1S)-1-carboxy-2-{4-[4- (ethoxymethyl)-2,6- dimethoxyphenyl]phenyl}ethyl] carbamoyl}-3,5-dichlorobenzoic acid | 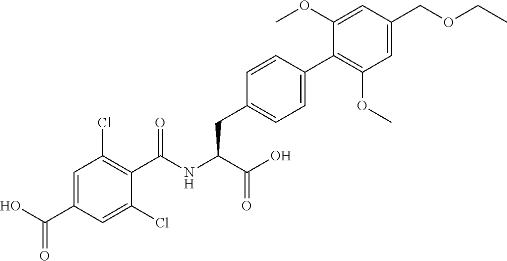 [SMILES: CCOCc1cc(OC)c(-c2ccc(C[C@H](NC(=O)c3c(Cl)cc(C(=O)O)cc3Cl)C(=O)O)cc2)c(OC)c1] | 52% | 13.74 (br. s, 1 H) 12.87 (br. s, 1 H) 9.22 (d, J = 8.31 Hz, 1 H) 7.85 (s, 2 H) 7.26 (d, J = 8.31 Hz, 2 H) 7.09 (d, J = 8.07 Hz, 2 H) 6.68 (s, 2 H) 4.76 (m, J = 2.20 Hz, 1 H) 4.47 (s, 2 H) 3.66 (s, 6 H) 3.53 (q, J = 7.09 Hz, 2 H) 3.20 (dd, J = 13.94, 4.40 Hz, 1 H) 2.91 (dd, J = 14.18, 10.27 Hz, 1 H) 1.19 (t, J = 6.97 Hz, 3 H) | 576.1, 578.0 |
| Cmpd 24 | 4-{4-[(2S)-2-carboxy-2-[(4- carboxy-2,6- dichlorophenyl)formamido]ethyl] phenyl}-3,5-dimethoxybenzoic acid | 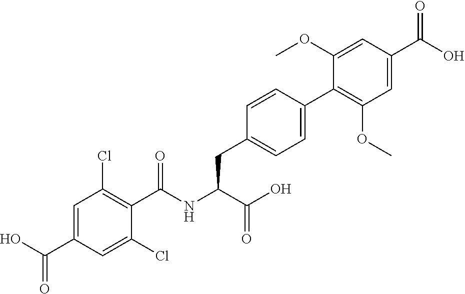 [SMILES: COc1cc(C(=O)O)cc(OC)c1-c1ccc(C[C@H](NC(=O)c2c(Cl)cc(C(=O)O)cc2Cl)C(=O)O)cc1] | 51% | 13.00 (br. s, 3 H) 9.22 (d, J = 8.31 Hz, 1 H) 7.85 (s, 2 H) 7.30 (d, J = 8.31 Hz, 2 H) 7.27 (s, 2 H) 7.13 (d, J = 8.07 Hz, 2 H) 4.71-4.83 (m, 1 H) 3.72 (s, 6 H) 3.22 (dd, J = 14.31, 4.28 Hz, 1 H) 2.92 (dd, J = 13.94, 10.76 Hz, 1 H) | 562.0, 564.0 |
| Cmpd 25 | 4-{[(1S)-1-carboxy-2-{4-[4-(2- hydroxypropan-2-yl)-2,6- dimethoxyphenyl]phenyl}ethyl] carbamoyl}-3,5-dichlorobenzoic acid | 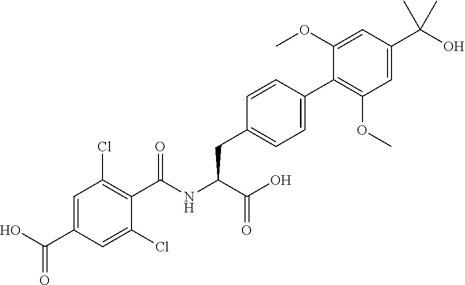 [SMILES: COc1cc(C(C)(C)O)cc(OC)c1-c1ccc(C[C@H](NC(=O)c2c(Cl)cc(C(=O)O)cc2Cl)C(=O)O)cc1] | 74% | 13.72 (br. s., 1 H) 12.88 (br. s., 1 H) 9.22 (d, J = 8.31 Hz, 1 H) 7.85 (s, 2 H) 7.25 (d, J = 8.07 Hz, 2 H) 7.09 (d, J = 8.31 Hz, 2 H) 6.81 (s, 2 H) 5.04 (s, 1 H) 4.67-4.82 (m, 1 H) 3.66 (s, 6 H) 3.20 (dd, J = 14.06, 4.03 Hz, 1 H) 2.91 (dd, J = 14.18, 10.51 Hz, 1 H) 1.47 (s, 6 H) | 576.1, 578.0 |
| Cmpd 26 | 4-{[(1S)-1-carboxy-2-{4-[4- (fluoromethyl)-2,6- dimethoxyphenyl]phenyl}ethyl] carbamoyl}-3,5-dichlorobenzoic acid | 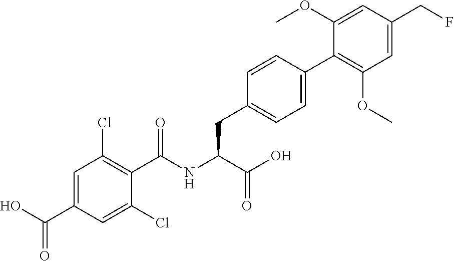 [SMILES: COc1cc(CF)cc(OC)c1-c1ccc(C[C@H](NC(=O)c2c(Cl)cc(C(=O)O)cc2Cl)C(=O)O)cc1] | 69% | 13.73 (br. s, 1 H) 12.89 (br. s, 1 H) 9.22 (d, J = 8.31 Hz, 1 H) 7.85 (s, 2 H) 7.28 (d, J = 8.07 Hz, 2 H) 7.10 (d, J = 8.07 Hz, 2 H) 6.80 (d, J = 1.47 Hz, 2 H) 5.49 (s, 1 H) 5.37 (s, 1 H) 4.77 (m, J = 2.40 Hz, 1 H) 3.68 (s, 6 H) 3.21 (dd, J = 14.18, 3.91 Hz, 1 H) 2.92 (dd, J = 14.06, 10.64 Hz, 1 H) | 550.0, 552.0 |
| Cmpd 33 | (S)-2-((S)-1-((3,5- dichlorophenyl)sulfonyl)-2- methylpyrrolidine-2- carboxamido)-3-(4′- (ethoxymethyl)-2′,6′-dimethoxy- [1,1′-biphenyl]-4-yl)propanoic acid | 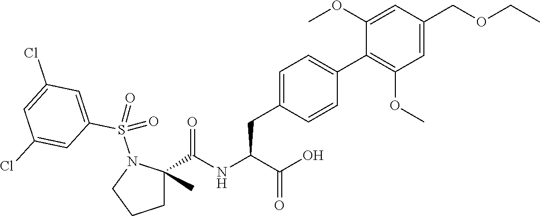 [SMILES: CCOCc1cc(OC)c(-c2ccc(C[C@H](NC(=O)[C@]3(C)CCCN3S(=O)(=O)c3cc(Cl)cc(Cl)c3)C(=O)O)cc2)c(OC)c1] | 66% | ¹H NMR (400 MHz, DMSO- d6) δ ppm 12.86 (br. s., 1 H) 7.97 (t, J = 1.96 Hz, 1 H) 7.81 (d, J = 1.71 Hz, 2 H) 7.70 (d, J = 7.82 Hz, 1 H) 7.20 (d, J = 8.31 Hz, 2 H) 7.09 (d, J = 8.31 Hz, 2 H) 6.66 (s, 2 H) 4.50 (m, J = 4.90 Hz, 1 H) 4.46 (s, 2 H) 3.62 (s, 6 H) 3.51 (q, J = 7.09 Hz, 2 H) 3.37 (m, J = 7.10 Hz, 2 H) 3.12- 3.22 (m, 1 H) 2.96-3.08 (m, 1 H) 1.92 (m, J = 7.30 Hz, 1 | 679.1, 681.1 |
| | | | | H) 1.67-1.80 (m, 2 H) 1.61- | |
| | | | | 1.69 (m, 1 H) 1.49 (s, 3 H) | |
| | | | | 1.18 (t, J = 6.97 Hz, 3 H). | |
| Cmpd 34 | (S)-3-(4′-(ethoxymethyl)-2′,6′- dimethoxy-[1,1′-biphenyl]-4-yl)- 2-((S)-2-methyl-1- (phenylsulfonyl)pyrrolidine-2- carboxamido)propanoic acid | 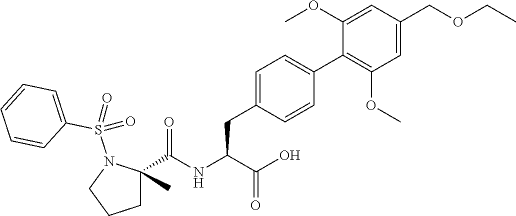 [SMILES: CCOCc1cc(OC)c(-c2ccc(C[C@H](NC(=O)[C@]3(C)CCCN3S(=O)(=O)c3ccccc3)C(=O)O)cc2)c(OC)c1] | 73% | ¹H NMR (400 MHz, DMSO- d6) δ ppm 12.90 (br. s., 1 H) 7.79-7.88 (m, 2 H) 7.61- 7.70 (m, 1 H) 7.50-7.60 (m, 3 H) 7.19 (d, J = 8.07 Hz, 2 H) 7.09 (d, J = 8.07 Hz, 2 H) 6.66 (s, 2 H) 4.52 (m, J = 4.90 Hz, 1 H) 4.46 (s, 2 H) 3.62 (s, 6 H) 3.52 (q, J = 7.01 Hz, 2 H) 3.37 (m, J = 5.10 Hz, 1 H) 3.23-3.30 (m, 1 H) 3.19 (dd, J = 13.94, 4.89 Hz, 1 H) 3.02 (dd, J = 1.00 Hz, 1 H) 1.86- | 611.2 |
| | | | | 1.97 (m, 1 H) 1.49-1.71 (m, | |
| | | | | 3 H) 1.43 (s, 3 H) 1.18 (t, | |
| | | | | J = 6.97 Hz, 3 H). | |
| Cmpd 35 | (S)-3-(4′-(ethoxymethyl)-2′,6′- dimethoxy-[1,1′-biphenyl]-4-yl)- 2-((S)-1- (phenylsulfonyl)pyrrolidine-2- carboxamido)propanoic acid | 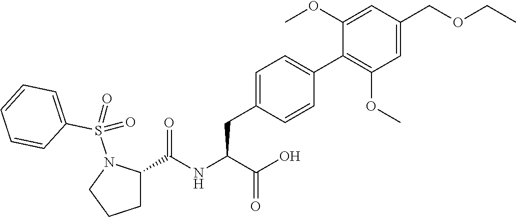 [SMILES: CCOCc1cc(OC)c(-c2ccc(C[C@H](NC(=O)[C@@H]3CCCN3S(=O)(=O)c3ccccc3)C(=O)O)cc2)c(OC)c1] | 93% | ¹H NMR (400 MHz, DMSO- d6) δ ppm 12.90 (br. s., 1 H) 8.17 (d, J = 8.07 Hz, 1 H) 7.82 (d, J = 7.58 Hz, 2 H) 7.65- 7.74 (m, 1 H) 7.55-7.64 (m, 2 H) 7.22 (d, J = 8.07 Hz, 2 H) 7.10 (d, J = 7.82 Hz, 2 H) 6.66 (s, 2 H) 4.47-4.55 (m, 1 H) 4.45 (s, 2 H) 4.14 (m, J = 7.60 Hz, 1 H) 3.61 (s, 6 H) 3.51 (q, J = 7.09 Hz, 2 H) 3.31- 3.38 (m, 1 H) 3.07-3.19 (m, 2 H) 2.94-3.05 (m, 1 H) | 597.2 |
| | | | | 1.46-1.65 (m, 3 H) 1.40 (m, | |
| | | | | J = 6.10 Hz, 1 H) 1.17 (t, | |
| | | | | J = 7.09 Hz, 3 H). | |
| Cmpd 36 | (S)-2-((S)-1-((3,5- dichlorophenyl)sulfonyl)pyrrolidine- 2-carboxamido)-3-(4′- (ethoxymethyl)-2′,6′-dimethoxy- [1,1′-biphenyl]-4-yl)propanoic acid | 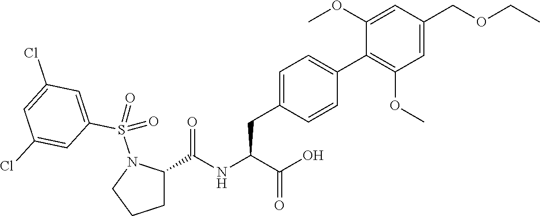 [SMILES: CCOCc1cc(OC)c(-c2ccc(C[C@H](NC(=O)[C@@H]3CCCN3S(=O)(=O)c3cc(Cl)cc(Cl)c3)C(=O)O)cc2)c(OC)c1] | 84% | ¹H NMR (400 MHz, DMSO- d6) δ ppm 12.89 (s, 1 H) 8.31 (d, J = 8.07 Hz, 1 H) 8.02 (s, 1 H) 7.85 (d, J = 1.47 Hz, 2 H) 7.23 (d, J = 7.82 Hz, 2 H) 7.10 (d, J = 7.83 Hz, 2 H) 6.66 (s, 2 H) 4.48-4.57 (m, 1 H) 4.46 (s, 2 H) 4.26-4.34 (m, 1 H) 3.62 (s, 6 H) 3.51 (q, J = 6.93 Hz, 2 H) 3.35-3.41 (m, 1 H) 3.23 (m, J = 9.30 Hz, 1 H) 3.06-3.15 (m, 1 H) 2.94-3.05 (m, 1 H) 1.61- | 665.1, 667.1 |
| | | | | 1.79 (m, 3 H) 1.54 (m, | |
| | | | | J = 4.90 Hz, 1 H) 1.18 (t, | |
| | | | | J = 6.97 Hz, 3 H). | |
| Cmpd 42 | (S)-3-(2′,6′-dimethoxy-4′-((2- methoxyethoxy)methyl)-[1,1′- biphenyl]-4-yl)-2-((S)-1- (phenylsulfonyl)azetidine-2- carboxamido)propanoic acid | 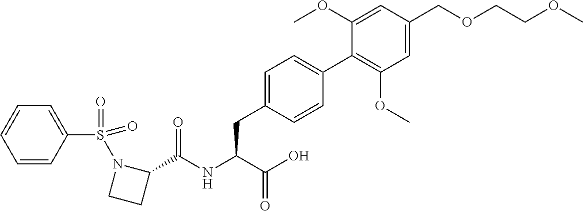 [SMILES: COCCOCc1cc(OC)c(-c2ccc(C[C@H](NC(=O)[C@@H]3CCN3S(=O)(=O)c3ccccc3)C(=O)O)cc2)c(OC)c1] | 62% | ¹H NMR (400 MHz, DMSO- d6) δ ppm 12.97 (br. s, 1 H) 8.18 (d, J = 8.07 Hz, 1 H) 7.81- 7.87 (m, 2 H) 7.74-7.80 (m, 1 H) 7.64-7.70 (m, 2 H) 7.23 (d, J = 8.07 Hz, 2 H) 7.12 (d, J = 8.31 Hz, 2 H) 6.68 (s, 2 H) 4.53 (m, J = 5.40 Hz, 1 H) 4.50 (s, 2 H) 4.30 (dd, J = 9.29, 7.34 Hz, 1 H) 3.67 (m, J = 4.40 Hz, 1 H) 3.62 (s, 6 H) 3.57-3.61 (m, 2 H) 3.55 (d, J = 9.05 Hz, 1 H) 3.48- | 613.2 |
| | | | | 3.53 (m, 2 H) 3.27 (s, 3 H) | |
| | | | | 3.13 (m, J = 5.10 Hz, 1 H) | |
| | | | | 3.03 (m, J = 8.30 Hz, 1 H) | |
| | | | | 2.00-2.11 (m, 1 H) 1.86- | |
| | | | | 1.98 (m, 1 H) | |
| Cmpd 43 | (S)-2-((S)-1-((3,5- dichlorophenyl)sulfonyl)azetidine- 2-carboxamido)-3-(2′,6′- dimethoxy-4′-((2- methoxyethoxy)methyl)-[1,1′- biphenyl]-4-yl)propanoic acid | 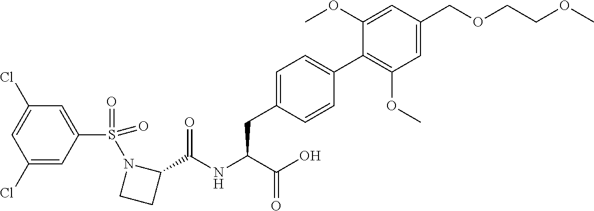 [SMILES: COCCOCc1cc(OC)c(-c2ccc(C[C@H](NC(=O)[C@@H]3CCN3S(=O)(=O)c3cc(Cl)cc(Cl)c3)C(=O)O)cc2)c(OC)c1] | 61% | ¹H NMR (400 MHz, DMSO- d6) δ ppm 12.97 (br. s, 1 H) 8.32 (d, J = 7.82 Hz, 1 H) 8.07 (t, J = 1.83 Hz, 1 H) 7.86 (d, J = 1.96 Hz, 2 H) 7.23 (d, J = 8.31 Hz, 2 H) 7.12 (d, J = 8.31 Hz, 2 H) 6.68 (s, 2 H) 4.54 (dd, J = 9.17, 7.21 Hz, 2 H) 4.50 (s, 2 H) 3.67-3.77 (m, 2 H) 3.62 (s, 6 H) 3.56- 3.61 (m, 2 H) 3.49-3.54 (m, 2 H) 3.28 (s, 3 H) 3.10 (m, J = 5.60 Hz, 1 H) 3.03 (m, | 703.1 705.1 (M + 23) |
| | | | | J = 8.10 Hz, 1 H) 2.14-2.25 | |
| | | | | (m, 1 H) 1.93-2.06 (m, 1 H) | |
| Cmpd 44 | (S)-3-(4′-(2,5,8,11- tetraoxadodecyl)-2′,6′- dimethoxy-[1,1′-biphenyl]-4-yl)- 2-((S)-1- (phenylsulfonyl)azetidine-2- carboxamido)propanoic acid | 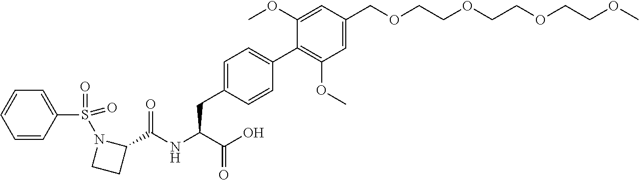 [SMILES: COCCOCCOCCOCc1cc(OC)c(-c2ccc(C[C@H](NC(=O)[C@@H]3CCN3S(=O)(=O)c3ccccc3)C(=O)O)cc2)c(OC)c1] | 85% | ¹H NMR (400 MHz, DMSO- d6) δ ppm 12.96 (br. s, 1 H) 8.18 (d, J = 8.07 Hz, 1 H) 7.81- 7.87 (m, 2 H) 7.74-7.81 (m, 1 H) 7.64-7.71 (m, 2 H) 7.24 (d, J = 8.07 Hz, 2 H) 7.12 (d, J = 8.07 Hz, 2 H) 6.68 (s, 2 H) 4.52-4.57 (m, 1 H) 4.51 (s, 2 H) 4.30 (dd, J = 9.29, 7.34 Hz, 1 H) 3.67 (m, J = 4.60 Hz, 1 H) 3.62 (s, 6 H) | 723.2 (M + 23) |
| | | | | 3.59 (s, 4 H) 3.48-3.57 (m, | |
| | | | | 7 H) 3.38-3.44 (m, 2 H) | |
| | | | | 3.22 (s, 3 H) 3.14 (dd, | |
| | | | | J = 1.00 Hz, 1 H) 3.03 (dd, | |
| | | | | J = 7.80 Hz, 1 H) 1.99-2.12 | |
| | | | | (m, 1 H) 1.86-1.98 (m, 1 H) | |
| Cmpd 45 | (S)-3-(4′-(2,5,8,11- tetraoxadodecyl)-2′,6′- dimethoxy-[1,1′-biphenyl]-4-yl)- 2-((S)-1-((3,5- dichlorophenyl)sulfonyl)azetidine- 2-carboxamido)propanoic acid | 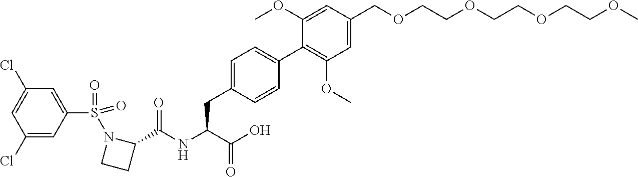 [SMILES: COCCOCCOCCOCc1cc(OC)c(-c2ccc(C[C@H](NC(=O)[C@@H]3CCN3S(=O)(=O)c3cc(Cl)cc(Cl)c3)C(=O)O)cc2)c(OC)c1] | 73% | ¹H NMR (400 MHz, DMSO- d6) δ ppm 12.96 (br. s, 1 H) 8.32 (d, J = 7.82 Hz, 1 H) 8.07 (t, J = 1.83 Hz, 1 H) 7.86 (d, J = 1.96 Hz, 2 H) 7.23 (d, J = 8.31 Hz, 2 H) 7.12 (d, J = 8.07 Hz, 2 H) 6.68 (s, 2 H) 4.52-4.58 (m, 2 H) 4.51 (s, 2 H) 3.65-3.80 (m, 2 H) 3.62 (s, 6 H) 3.59 (s, 4 H) 3.47-3.57 (m, 6 H) 3.39- | 791.1 793.1 (M + 23) |
| | | | | 3.44 (m, 2 H) 3.22 (s, 3 H) | |
| | | | | 3.11 (dd, J = 5.10 Hz, 1 H) | |
| | | | | 3.03 (dd, J = 8.10 Hz, 1 H) | |
| | | | | 2.12-2.25 (m, 1 H) 1.92- | |
| | | | | 2.07 (m, 1 H) | |
| Cmpd 49 | 4-(((S)-2-(((S)-1-carboxy-2- (2′,6′-dimethoxy-4′-((2- methoxyethoxy)methyl)-[1,1′- biphenyl]-4- yl)ethyl)carbamoyl)pyrrolidin-1- yl) sulfonyl)benzoic acid | 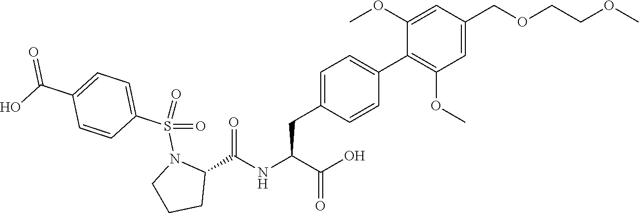 [SMILES: COCCOCc1cc(OC)c(-c2ccc(C[C@H](NC(=O)[C@@H]3CCCN3S(=O)(=O)c3ccc(C(=O)O)cc3)C(=O)O)cc2)c(OC)c1] | 62% | ¹H NMR (400 MHz, DMSO- d6) δ ppm 13.20 (br. s, 2 H) 8.19 (d, J = 8.07 Hz, 1 H) 8.03- 8.12 (m, 2 H) 7.86-7.91 (m, 2 H) 7.23 (d, J = 8.31 Hz, 2 H) 7.11 (d, J = 8.07 Hz, 2 H) 6.67 (s, 2 H) 4.43-4.53 (m, 3 H) 4.18 (dd, J = 7.09, 3.91 Hz, 1 H) 3.61 (s, 6 H) 3.56- 3.60 (m, 2 H) 3.47-3.53 (m, 2 H) 3.27 (s, 3 H) 3.07-3.22 (m, 3 H) 3.02 (m, J = 9.00 Hz, 1 H) 1.54-1.67 (m, 3 H) | 671.3 (M + H) |
| | | | | 1.40-1.52 (m, 1 H) | |

VCAM-1 Assay Results

Flow Cytometry Cell-Based Assay.

Compounds were tested for their ability to inhibit the binding of soluble VCAM-1 to human G2 acute lymphoblastic leukemia (ALL) cells. Briefly, G2 ALL cells are pre-incubated with increasing concentrations (0.001 to 1000 nM) of compounds for 30 minutes. Soluble VCAM/Fc chimera protein (R&D systems) is then added to the mixture and the cells incubated for an additional 30 minutes. Afterwards, cells are washed and VCAM-1 is detected using PE-conjugated secondary mAbs. In each experiment, an aliquot of cells will be stained with isotype control mAbs to serve as a negative control. The percentage of VCAM-1 binding cells was then determined by flow cytometry.

TABLE 12
Inhibition of sVCAM-1 binding to human G2 ALL cells.

| Example | sVCAM-1 IC₅₀ (nM) |
|---|---|
| Firategrast | 230 |
| 12 | 3.33 |
| 21 | 72 |
| 22 | 0.47 |
| 23 | 0.58 |
| 24 | 218 |
| 25 | 70 |
| 26 | 1.57 |
| 33 | 2.47 |
| 34 | 11.7 |
| 35 | 0.83 |
| 36 | 0.49 |

CFC Mobilization by Combination Therapies

Mice.

DBA/2J, C57BL/6J (CD45.2) and syngeneic B6.SJL-Ptprc^{a} Pep3^{b}/BoyJ (CD45.1) mice were purchased from the Jackson Laboratory (Bar Harbor, Me., USA). F1-hybrid mice (CD45.1/2) were obtained through breeding CD45.2 and CD45.1 mice. Animals were housed at the Washington University Medical School vivarium under SPF conditions. All experiments were performed in accordance with the guidelines of the Washington University Animal Studies Committee and the institutional animal care and use committee (IACUC), in agreement with AAALAC guidelines. Following lethal irradiation (1×9.5 or 11.0 Gy, using a ¹³⁷Cesium source) and transplantation, mice were kept on antibiotic medication, sulfamethoxazole and trimethoprim, 0.5 and 0.1 mg/mL respectively, (Hi-Tech Pharmacal, Amityville, N.Y., USA) peroral in drinking water.

Hspc Mobilization.

Recombinant human CXCL2 (Groβt; R&D systems) was reconstituted in sterile Ca⁺²/Mg⁺²-free phosphate buffered saline (PBS) and injected subcutaneously (SC) at a dose of 2.5 mg/kg. Firategrast (published structure: C₂₇H₂₇F2NO₆; U.S. Patent Application No. 2014/051655) was dissolved in Ca⁺²/Mg⁺²-free PBS plus 1% ethanol and injected intravenously at a dose of 100 mg/kg. RhG-CSF (Neupogen®, Filgrastim, Amgen, Thousand Oaks, Calif., USA) diluted in PBS was injected i.p. every 12 hrs at a dose of 100 μg/kg for a total of 9 (day 5) doses.

Colony Forming Cell (CFC) Assay.

Peripheral blood (PB) was drawn from the facial vein without anaesthesia into K/EDTA anti-coagulated tubes (Sarstedt AG & Co, Nimbrecht, Germany). Red blood cells were removed from 25 μL aliquots of blood using hypotonic lysis (Ammonium-Chloride-Potassium, ACK buffer, 5-10 min at RT) and samples were mixed with 2.5 mL methylcellulose media supplemented with a cocktail of recombinant cytokines (MethoCult 3434; Stem Cell Technologies, Vancouver, BC, Canada). Cultures were plated in duplicate in 35 mm dishes and placed in a humidified chamber with 5% CO₂ at 37° C. After 7 d of culture, colonies containing at least 50 cells were counted using an inverted microscope in a blinded fashion.

Flow Cytometric Enumeration of Murine HSPCs.

Peripheral blood (PB) was drawn from the facial vein without anaesthesia into K/EDTA anti-coagulated tubes (Sarstedt AG & Co, Nimbrecht, Germany). Cell counts (WBC) were determined using an automatic hemocytometer (Hemavet 950, Drew Scientific, Dallas, Tex.). Red blood cells were removed from 25 μL aliquots of blood using hypotonic lysis (Ammonium-Chloride-Potassium, ACK buffer, 5-10 min at RT). Samples were resuspended in staining buffer (PBS supplemented with 0.5% bovine serum albumin and 2 mM EDTA) and incubated for 30 min at 4° C. with pre-titrated saturating dilutions of the following fluorochrome-labeled monoclonal antibodies (BD Biosciences, San Jose, Calif.; clone designated in parenthesis): Sca1-PE (D7), CD117-BV421 (ACK2), lineage-APC (17A2, RB6-8C5, RA3-6B2, Ter-119, M/170) and CD45-A700 (30-F11). Dead cells were excluded from these assays by staining with 2 μg/mL 7-amino-actinomycin D (Molecular Probes, Eugene, Oreg.) for 5 min prior to analysis. Appropriate isotype-matched negative controls were used to assess background fluorescence intensity. Samples were analyzed on a Beckman Coulter Gallios flow cytometer and data were analyzed using FlowJo software (TreeStar, Ashland, Oreg.). Absolute numbers of immunophenotypically defined populations (e.g. LSK) were calculated by identifying CD45 positive cells as WBC from the corresponding cell count analysis. Statistical comparisons of flow cytometry data were performed using an unpaired two-tailed Student t-test (GraphPad Prism). P-values ≤0.05 were considered significant.

Repopulating Unit (RU) Assay.

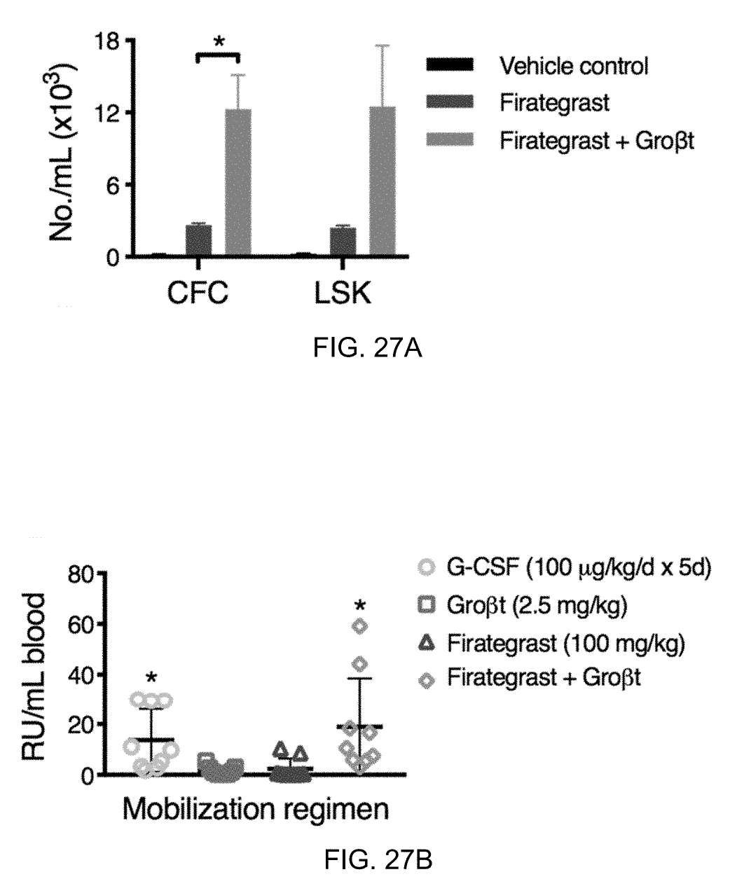

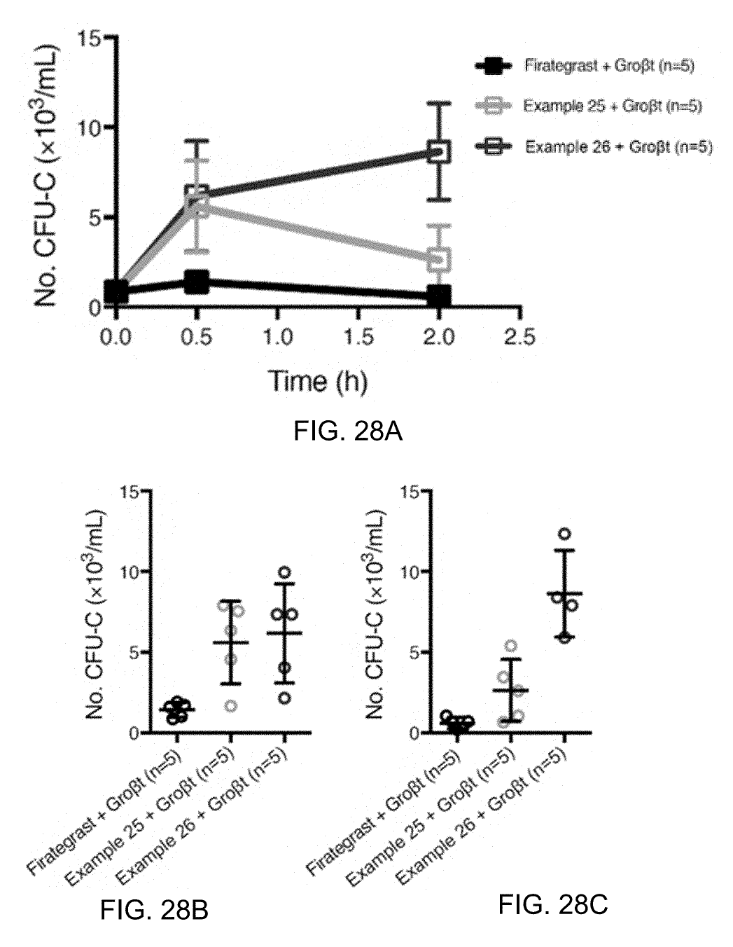

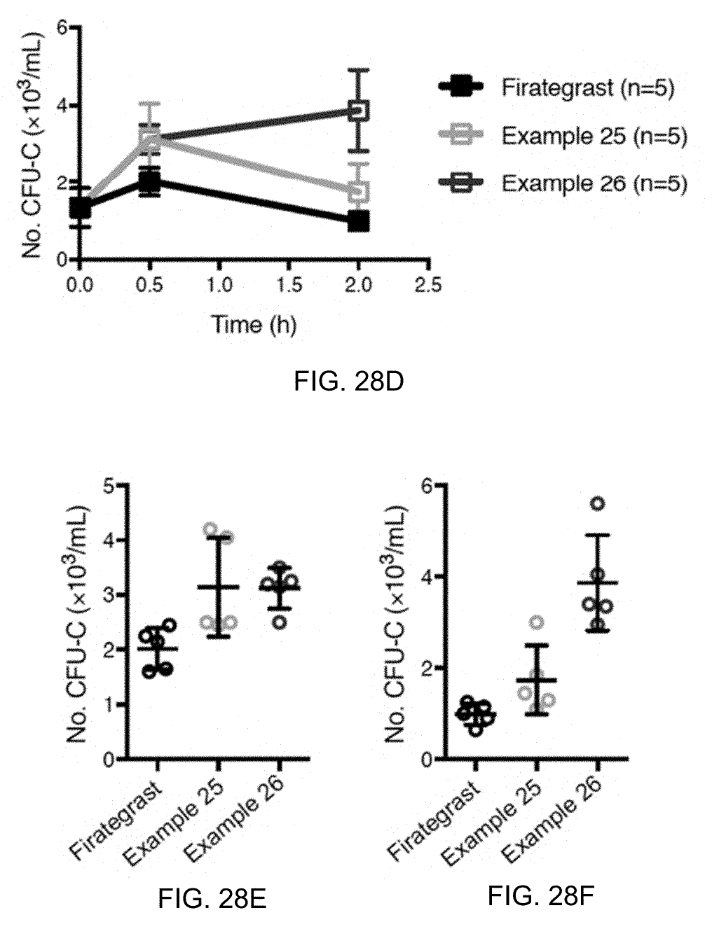

An RU assay was performed to directly compare the repopulating capacity of peripheral blood mobilized with different combinations of compounds. Lethally irradiated CD45.2 hosts received transplants consisting of 6 μL of blood from mobilized mice (CD45.1+) together with 2.5×10⁵ CD45.2+BM competitor cells. After 20 weeks, blood graft-derived RUs were calculated for the recipient mice by the following formula: RU=(D×C)/(100−D) where D is the percentage of blood-derived B lymphocytes and myeloid cells and C is the number of RUs contained in the competitor BM fraction of the graft (C=2.5 for 250,000 competitor BM cells). The results of these test are shown in FIG. 27 and FIG. 28.

### Example 4—Additional Studies of Combination of VLA-4 Inhibitors

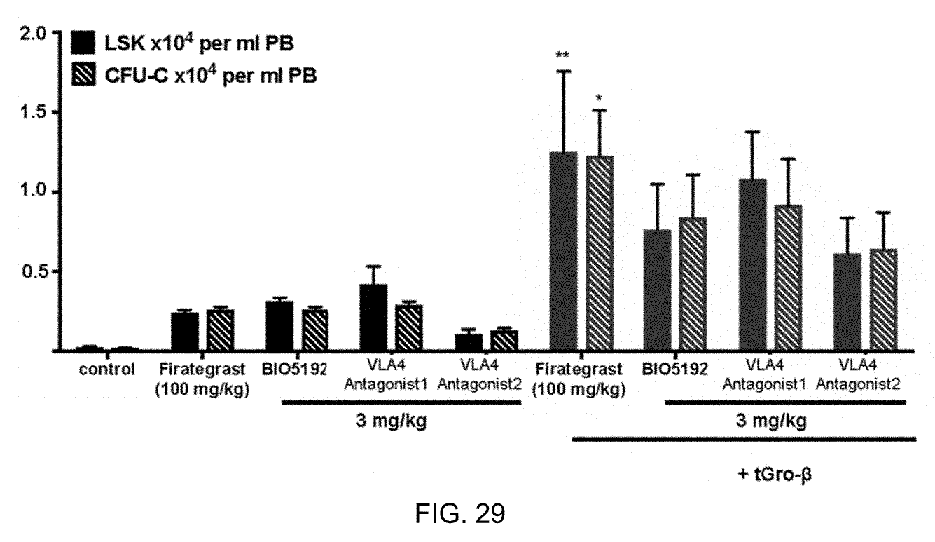

Using the combination of VLA4 blockade with CXCR2 stimulation, both CFU-C and LSK cells were mobilized. See FIG. 29.

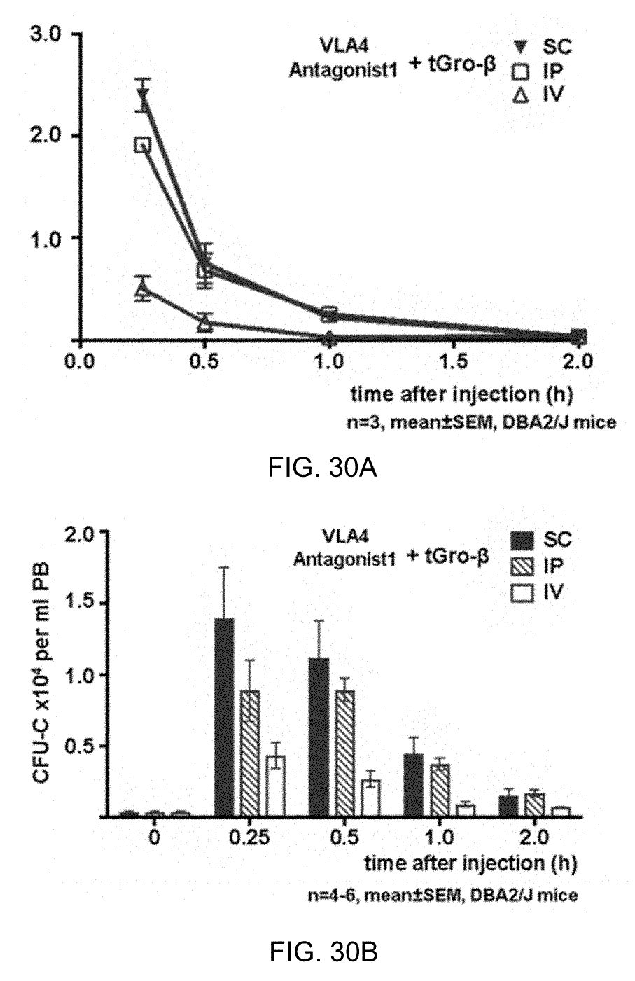

Additionally, the pharmacokinetics and pharmacodynamics of these combinations result in the fast mobilization of CFU-C per mL of peripheral blood. Three routes of administration were tested: subcutaneous, intraperitoneal, and intravenous administration. Subcutaneous and intraperitoneal showed higher CFU-C per mL of peripheral blood relative to intravenous administration and all routes of administration showed fast mobilization with the peak happening within 30 minutes of administration in DBA2/J mice. See FIGS. 30A & 30B.

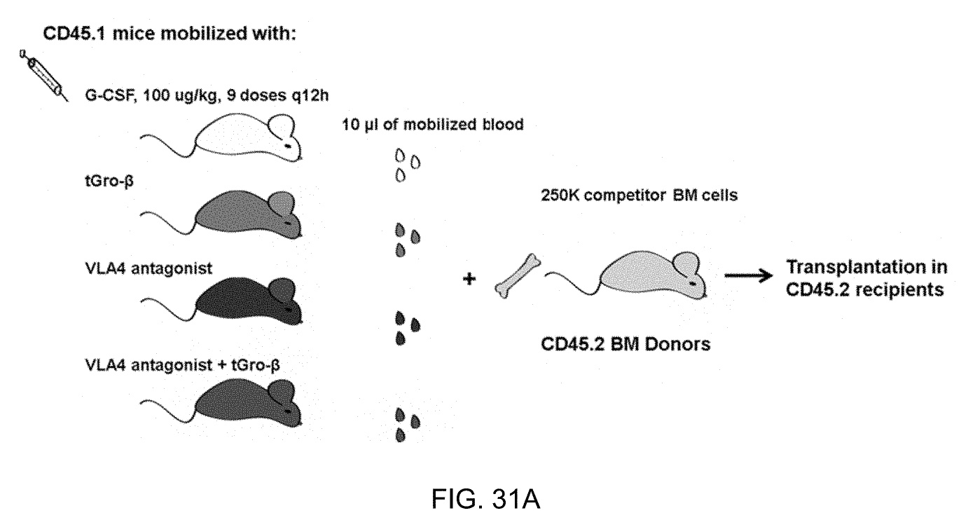

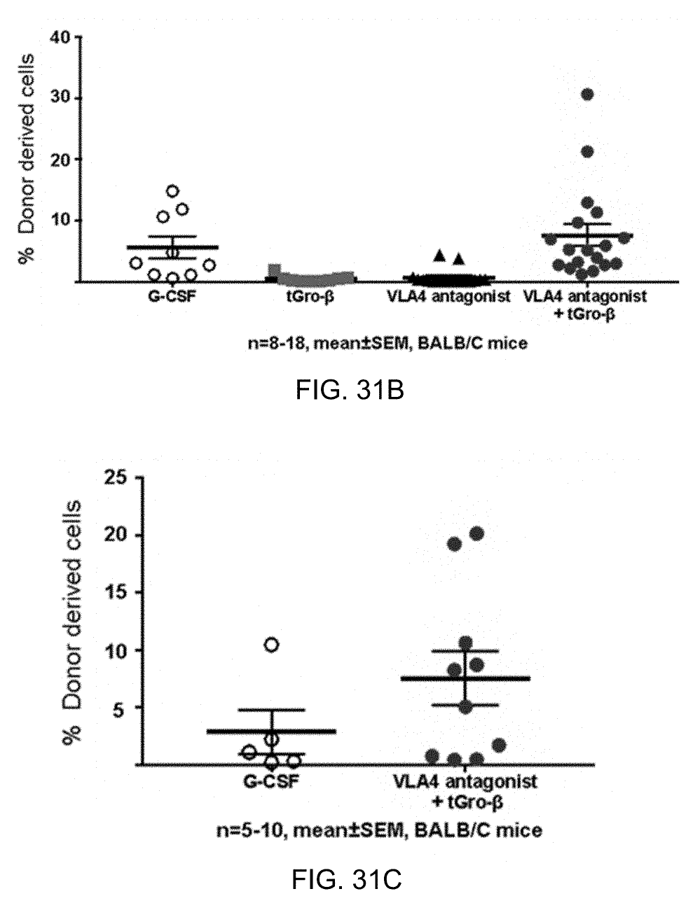

As can be seen in FIGS. 31A & 31B, the repopulation unit assay shows that the mobilization methods described herein using a combination of a VLA4 antagonist and tGro-β result in mobilized stem cells which can be successfully transplanted in both primary and secondary recipients.

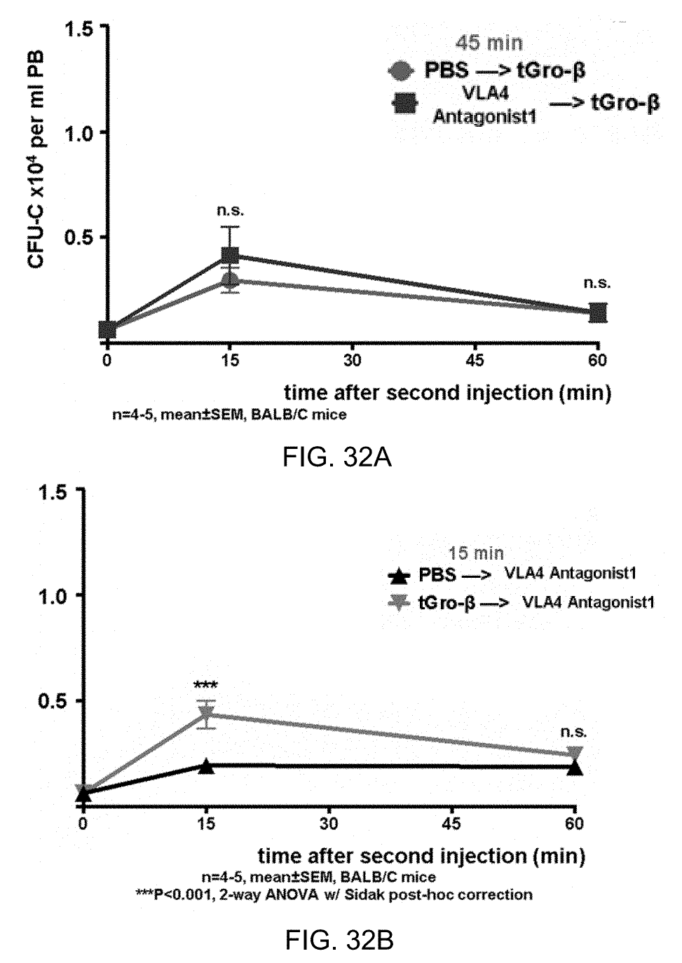

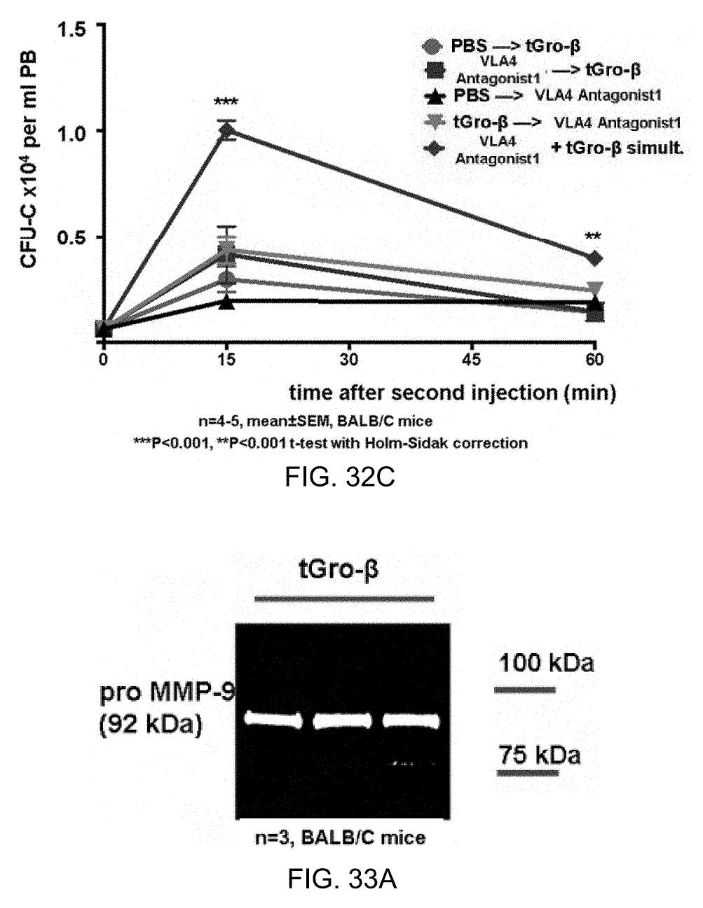

To determine if the order of administration matters, examples were the order of administration was varied. As can be seen in FIGS. 32A-32C, the highest number of colony forming units were obtained when the two agents were administered simultaneously. Without wishing to be bound by any theory, it is believed that CXCR2 stimulation primes the biological system for VLA4 blockage.

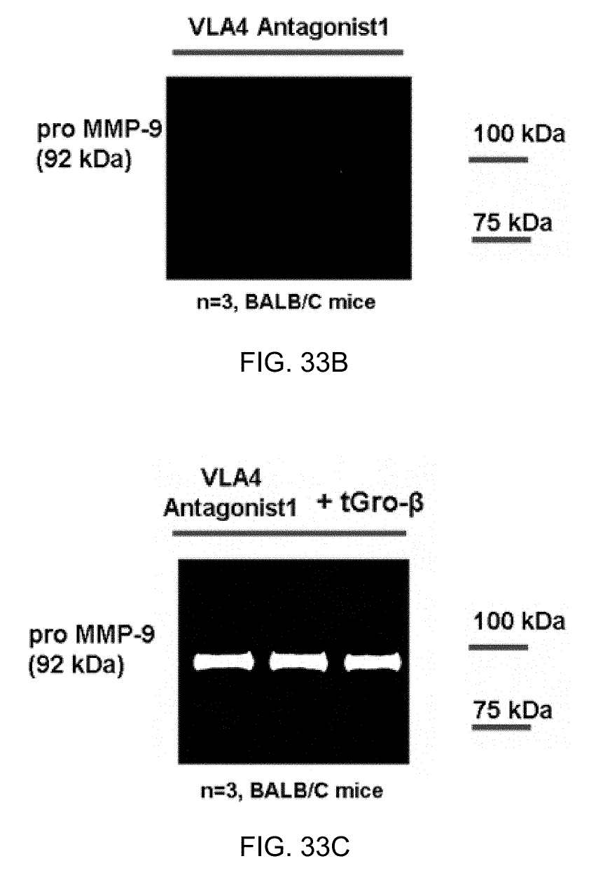

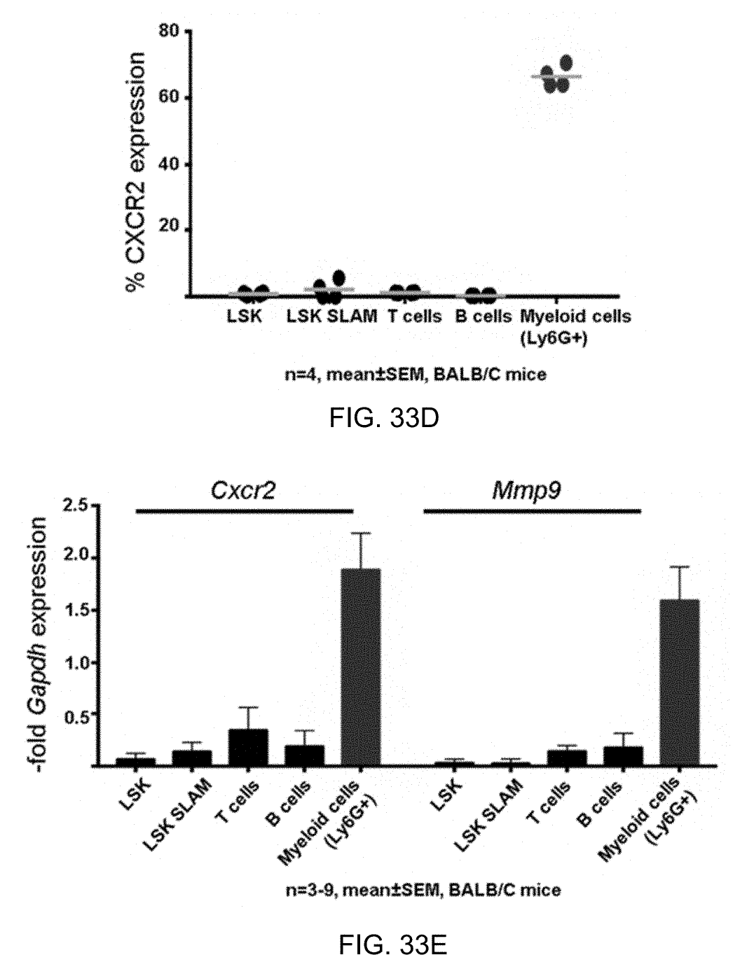

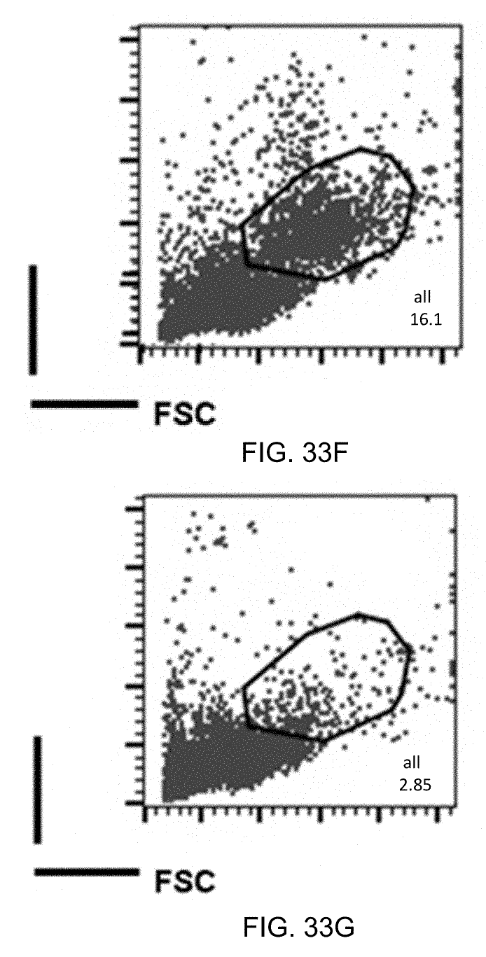

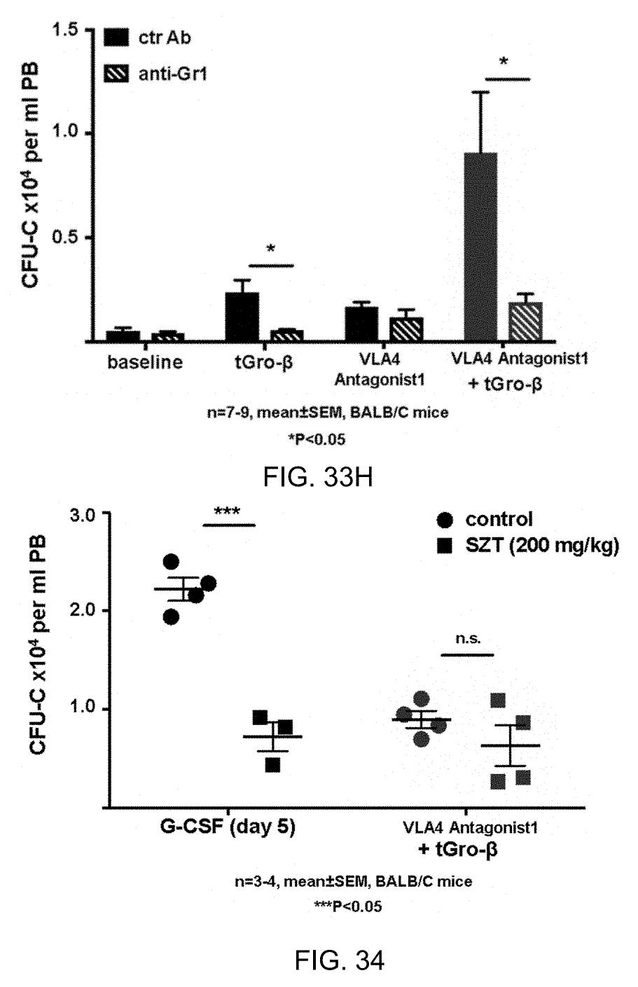

In order to determine the specific mechanism of action, studies were carried out to determine if MMP9 proteolytic activity was present in the compositions. In FIG. 33A, compositions which contained tGro-β showed MMP9 activity. Using both flow cytometry and real-time PCR it was determined that myeloid cells express both MMP9 and CXCR2 and may play a role in the mobilization of the desired stem cells. See FIG. 33B. In an alternative example where GR1+ myeloid cells were removed through the use of a GR1+ antibody, the mobilization of CFU-C was greatly decreased relative to a control antibodies (FIG. 33C). Without wishing to be bound by any theory, it is believed that GR1+ myeloid cells play a role in stem cell mobilization.

Finally, studies were carried out to determine the effectiveness of the combination in conditions which are typically difficult to achieve mobilization such as diabetes. Using a streptozotocin induced diabetes model, the combination of a VLA-4 inhibitor and tGro-β resulted in similar mobilization regardless of the nature of the model animal (FIG. 34). Thus, the combinations described herein may be especially useful in conditions which are difficult to mobilize hematopoietic stem or progenitor cells.

Triple Combination Therapy with VLA-4 Inhibitor+ CXCR4 Inhibitor+ CXCR2 Agonist

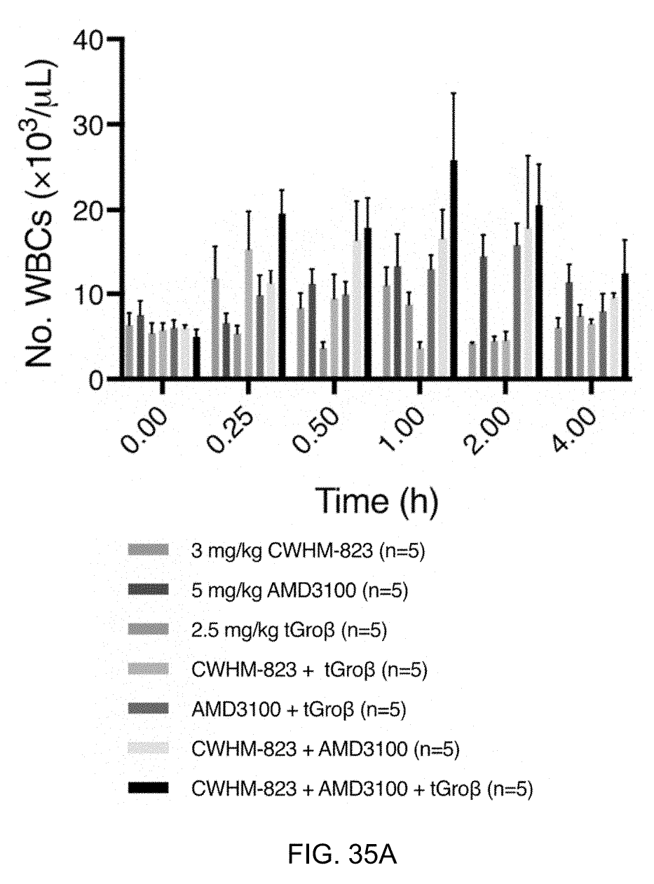

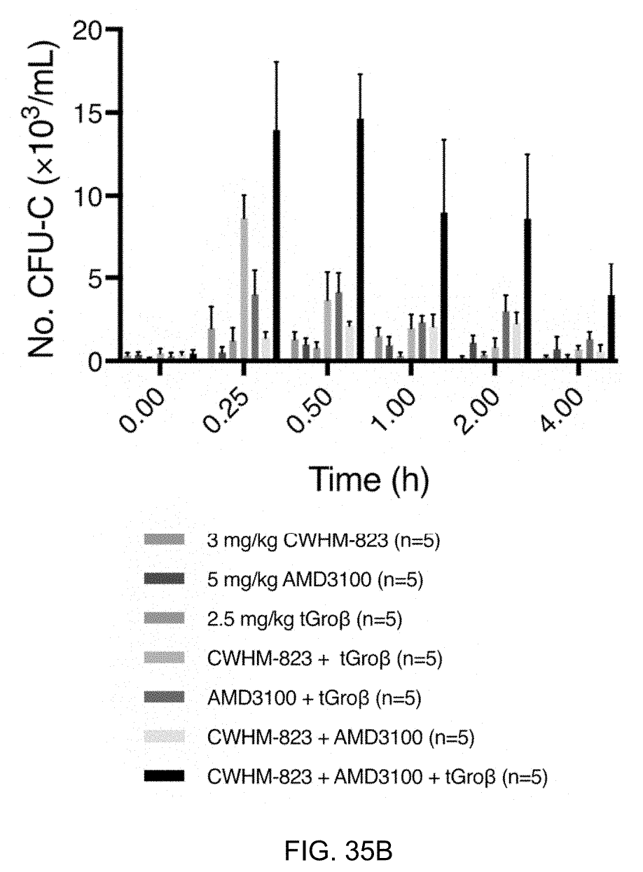

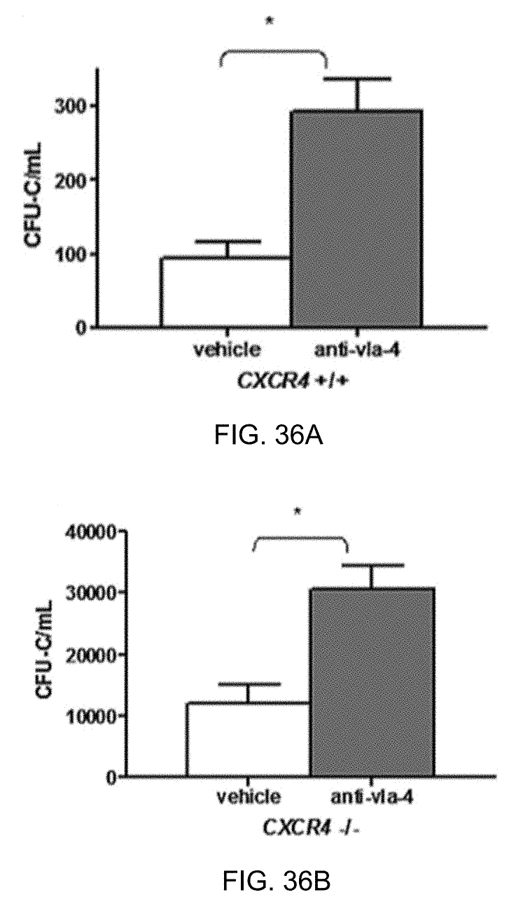

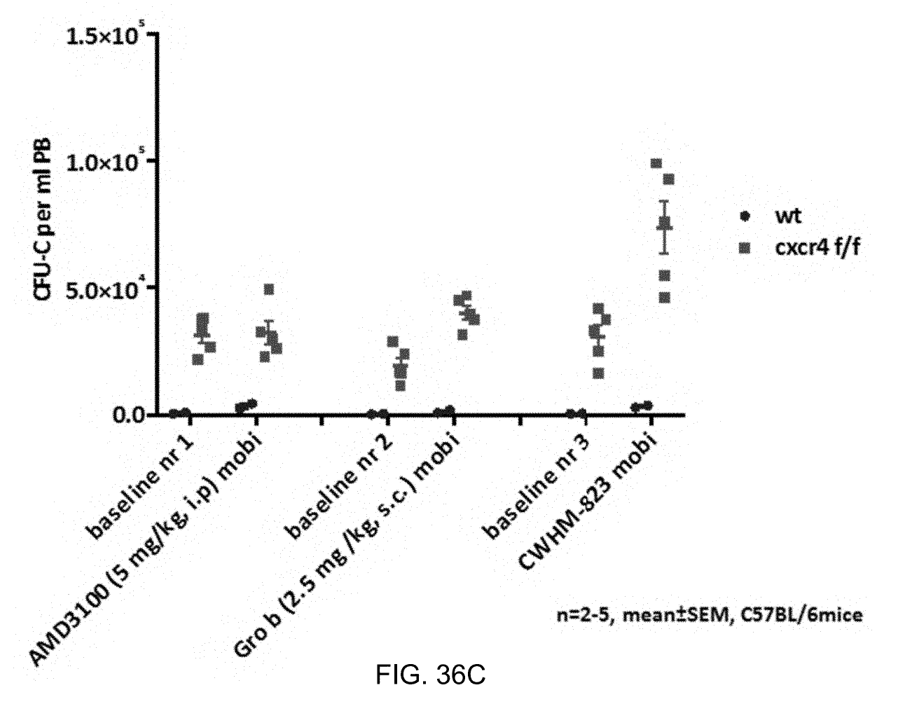

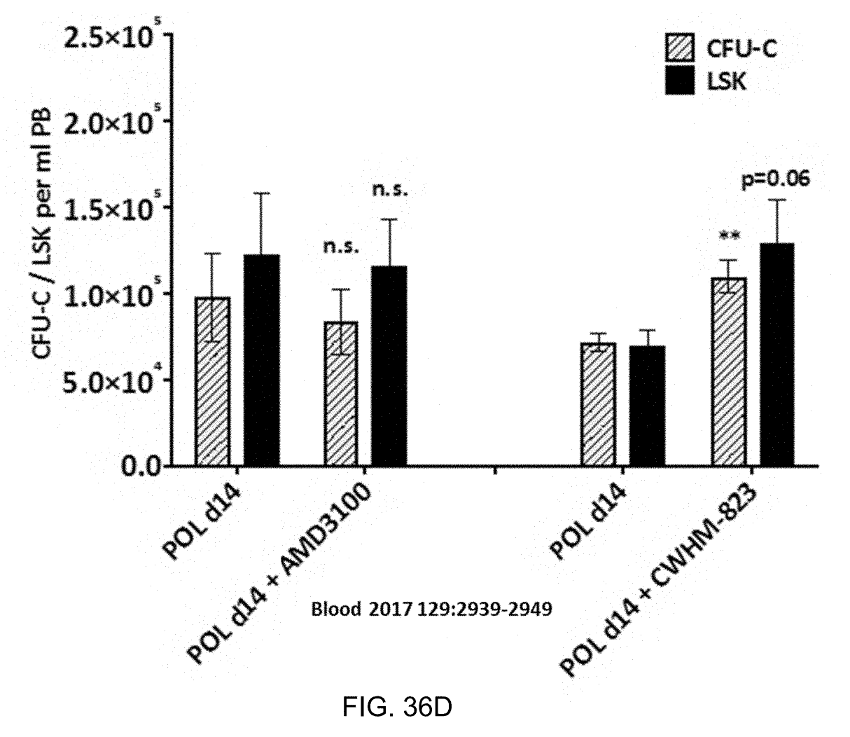

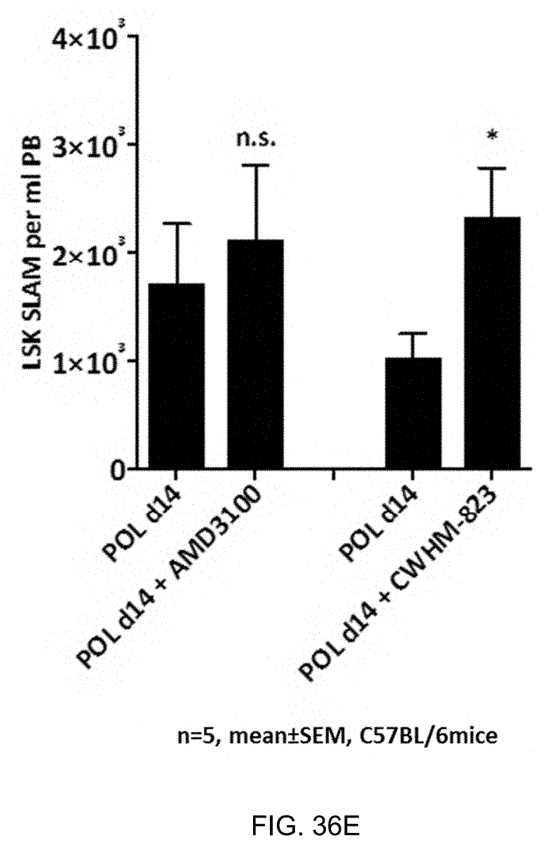

To test the mobilization efficacy of combination therapy with VLA-4 inhibitor+ CXCR4 inhibitor+ CXCR2 agonist BALB/c mice were treated with a variety of compositions. The mice were separated into seven groups: A) 3 mg/kg CWHM-823 (SC, n=5), B) 5 mg/kg AMD3100 (SC, n=5), C) 2.5 mg/kg tGroβ (SC, n=5), D) 3 mg/kg CWHM-823+2.5 mg/kg tGroβ (SC, n=5), E) 5 mg/kg AMD3100+2.5 mg/kg tGroβ (SC, n=5), F) 5 mg/kg AMD3100+3 CWHM-823 (SC, n=5) and G) 3 mg/kg CWHM-823+5 mg/kg AMD3100+2.5 mg/kg tGroβ (SC, n=5). On day 0, mice were inject mice as indicated above. Peripheral blood was collected from the mice at baseline, 0.25, 0.5, 1, 2 and 4 hours post-injection. Aliquots of blood were removed and stored for the CFU assay and Hemavet. On day +7 CFUs were counted. As can be seen in FIG. 35A and FIG. 35B, the triple combination of combination therapy with VLA-4 inhibitor+ CXCR4 inhibitor+ CXCR2 agonist provided significantly improved results over the other conditions tested.

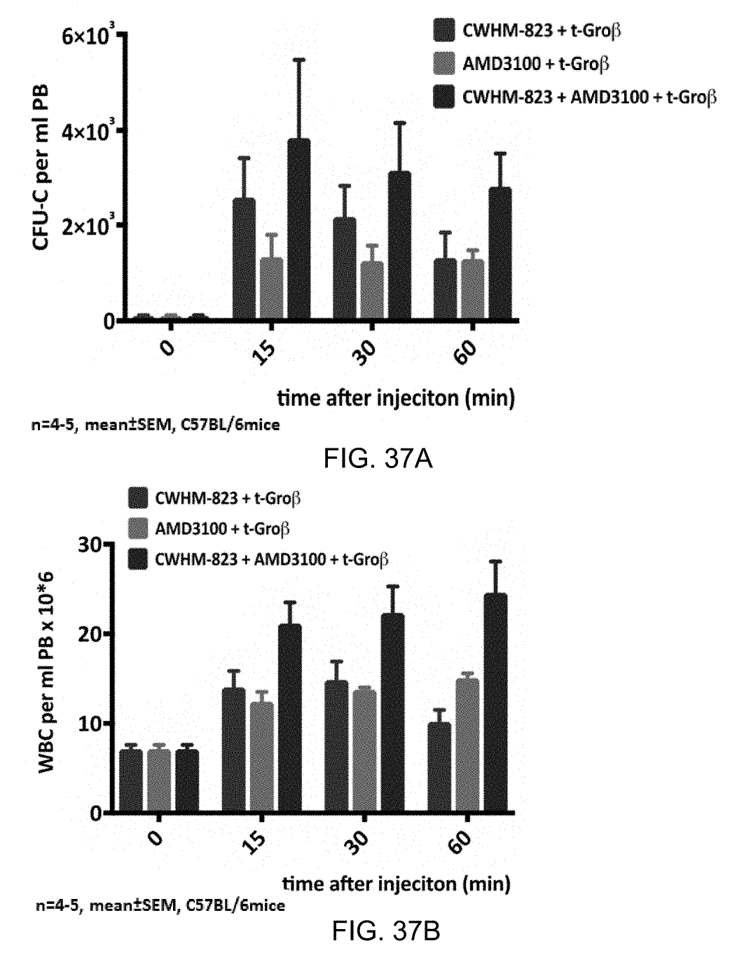

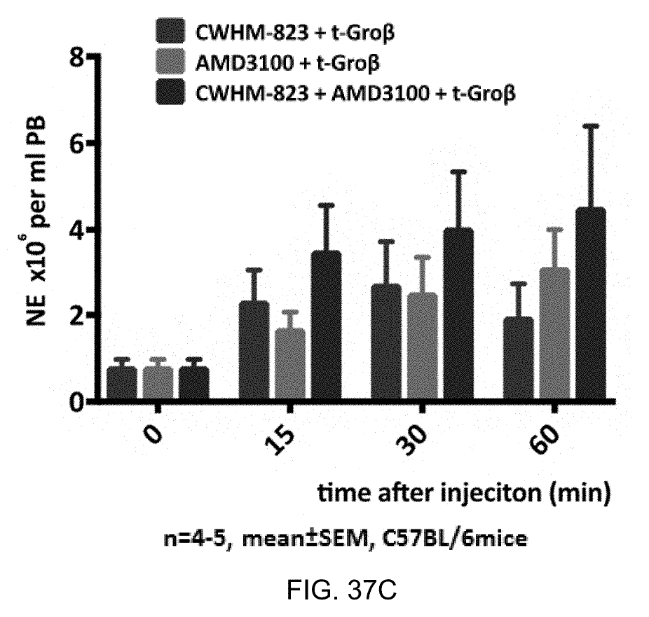

To further test the combination therapy with VLA-4 inhibitor+ CXCR4 inhibitor+ CXCR2 agonist BL/6 mice were administered simultaneous subcutaneous injection of; A) CWHM-823 (3 mg/kg)+t-Groβ (2.5 mg/kg), B) AMD3100 (5 mg/kg)+t-Groβ (2.5 mg/kg), or C) CWHM-823 (3 mg/kg)+AMD3100 (5 mg/kg)+t-Groβ (2.5 mg/kg). As can be seen in FIG. 37A-37B, the triple combination surprisingly outperforms compositions with a VLA-4 inhibitor and CXCR2 agonist or CXCR4 inhibitor and CXCR2 agonist.

All of the compounds, compositions, and methods disclosed and claimed herein can be made and executed without undue experimentation in light of the present disclosure. While the disclosure may have focused on several embodiments or may have been described in terms of preferred embodiments, it will be apparent to those of skill in the art that variations and modifications may be applied to the compounds, compositions, and methods without departing from the spirit, scope, and concept of the invention. All variations and modifications apparent to those skilled in the art are deemed to be within the spirit, scope, and concept of the invention as defined by the appended claims.

### REFERENCES

The following references to the extent that they provide exemplary procedural or other details supplementary to those set forth herein, are specifically incorporated herein by reference.
U.S. Patent Application No. 2014/0051655
Greene & Wuts, Protective Groups in Organic Synthesis, 3^{rd} Ed., John Wiley, 1999.
Handbook of Pharmaceutical Salts: Properties, and Use, Stahl and Wermuth (Eds.), Verlag Helvetica Chimica Acta, 2002.
March's Advanced Organic Chemistry: Reactions, Mechanisms, and Structure, 2007.
Reagan-Shaw et al., FASEB J., 22(3):659-661, 2008
EP 0842943
Greene & Wuts, Protective Groups in Organic Synthesis, 3rd Ed., John Wiley, 1999.
Handbook of Pharmaceutical Salts: Properties, and Use, Stahl and Wermuth (Eds.), Verlag Helvetica Chimica Acta, 2002.
Hepburn et al., Journal of Pharmacology and Experimental Therapeutics; 298: 886-893, 2001
King et al., Blood; 97:1534-1542, 2001.
King et al., J Immunol; 164: 3774-3782, 2000.
March's Advanced Organic Chemistry: Reactions, Mechanisms, and Structure, 2007.
PCT publication 99/26645
PCT publication WO 02/02132
PCT publication WO 94/29341
PCT publication WO 97/15594
PCT publication WO 97/15595
Reagan-Shaw et al., FASEB J., 22(3):659-661, 2008
Remington's Pharmaceutical Sciences, latest edition, Mack Publishing Company, Easton, Pa.
Smith and Williams Introduction to the Principles of Drug Design, Smith, H. J.; Wright, 2nd ed., London (1988).
U.S. 2014/051655
U.S. Pat. No. 5,021,409
U.S. Pat. No. 5,510,332
U.S. Pat. No. 5,583,131
U.S. Pat. No. 6,001,826
U.S. Pat. No. 6,080,398
U.S. Pat. No. 6,399,053
U.S. Pat. No. 6,447,766
U.S. Patent Application No. 2003/9229341
U.S. Patent Application No. 2005/027767
U.S. Patent Application No. 2005/059702
U.S. Patent Application No. 2014/0051655
WO 01/12186
WO 01/32610
WO 01/51487
WO 01/85196
WO 01/85196
WO 01/94420
WO 02/02132
WO 02/14272
WO 03/011288
WO 03/090512
WO 05/087760
WO 06/010054
WO 06/023396
WO 94/29341
WO 95/15973
WO 96/01644
WO 96/06108
WO 96/20216
WO 96/22966
WO 96/31206
WO 96/40781
WO 97/02289
WO 97/03094
WO 97/15594
WO 97/15595
WO 99/26645
WO 99/37605
WO 99/50461

What is claimed is:

1. A compound of the formula: 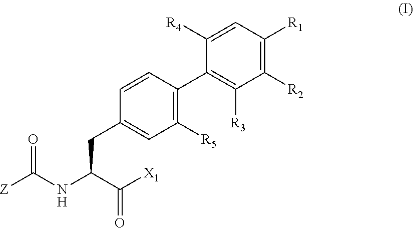
wherein:
R₁ is hydrogen, aminocarbonyl, carboxy, cyano, halo, hydroxy, —SF₅, alkyl_{(C≤8)}, substituted alkyl_{(C≤8)}, alkoxy_{(C≤8)}, substituted alkoxy_{(C≤8)}, or —Y₁—Rₐ; wherein:
Y₁ is alkanediyl_{(C≤8)} or substituted alkanediyl_{(C≤8)}; and
Rₐ is alkoxy_{(C≤12)}, acyloxy_{(C≤12)}, or a substituted version of either of these groups; or
—X(CH₂O)ₘ—(CH₂CH₂O)ₙ—R_{b}; wherein:
X is a covalent bond or —O—;
m is 0 or 1;
n is 1, 2, 3, 4, 5, 6, 7, or 8; and
R_{b} is hydrogen, alkyl_{(C≤8)}, or substituted alkyl_{(C≤8)}; or
—CH₂—O—(CH₂)ₙ—R_{c}; wherein:
N is 0, 1, or 2; and
R₀ is a cyclic aliphatic ring of 4-6 in size having one oxygen as part of the ring;
R₂ is hydrogen, aminocarbonyl, carboxy, cyano, halo, hydroxy, —SF₅, alkyl_{(C≤8)}, substituted alkyl_{(C≤8)}, alkoxy_{(C≤8)}, substituted alkoxy_{(C≤8)}, or —Y₂—R_{c}; wherein:
Y₂ is alkanediyl_{(C≤8)} or substituted alkanediyl_{(C≤8)}; and
R_{c} is alkoxy_{(C≤12)}, acyloxy_{(C≤12)}, or a substituted version of either of these groups;
X₁ is hydroxy, alkoxy_{(C≤8)}, substituted alkoxy_{(C≤8)}, cycloalkoxy_{(C≤8)}, substituted cycloalkoxy_{(C≤8)}, alkenyloxy_{(C≤8)}, substituted alkenyloxy_{(C≤8)}, aryloxy_{(C≤8)}, substituted aryloxy_{(C≤8)}, aralkoxy_{(C≤8)}, substituted aralkoxy_{(C≤8)}, or a substituent convertible in vivo to hydroxy; and
R₃ and R₄ are each independently hydrogen, hydroxy, alkoxy_{(C≤8)} or substituted alkoxy_{(C≤8)};
R₅ is hydrogen, —CH(OR_{d})Rₑ, or —C(O)R_{f}, wherein:
R_{d} is hydrogen, alkyl_{(C≤8)}, or substituted alkyl_{(C≤8)}; and
Rₑ and R_{f} are each independently alkyl_{(C≤8)} or substituted alkyl_{(C≤8)}; and
Z is a group of the formula: 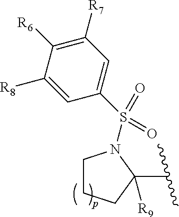
wherein:
p is 0, 1, 2, or 3;
R₆ is hydrogen or —C(O)X₂; wherein:
X₂ is amino, hydroxy, alkoxy_{(C≤8)}, substituted alkoxy_{(C≤8)}, cycloalkoxy_{(C≤8)}, substituted cycloalkoxy_{(C≤8)}, alkenyloxy_{(C≤8)}, substituted alkenyloxy_{(C≤8)}, aryloxy_{(C≤8)}, substituted aryloxy_{(C≤8)}, aralkyloxy_{(C≤8)}, substituted aralkyloxy_{(C≤8)}, alkylamino_{(C≤8)}, substituted alkylamino_{(C≤8)}, dialkylamino_{(C≤8)}, substituted dialkylamino_{(C≤8)}, cycloalkylamino_{(C≤8)}, substituted cycloalkylamino_{(C≤8)}, alkenylamino_{(C≤8)}, substituted alkenylamino_{(C≤8)}, arylamino_{(C≤8)}, substituted arylamino_{(C≤8)}, aralkylamino_{(C≤8)}, substituted aralkylamino_{(C≤8)}, or a substituent convertible in vivo to hydroxy;
R₇ and R₈ are each independently hydrogen, halo, haloalkyl_{(C≤8)}, or substituted haloalkyl_{(C≤8)};
R₉ is hydrogen, alkyl_{(C≤8)}, or substituted alkyl_{(C≤8)}; or
a group of the formula: 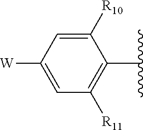
wherein:
W is hydrogen, cyano, halo, hydroxy, or —C(O)X₃, wherein:
X₃ is amino, hydroxy, alkoxy_{(C≤8)}, substituted alkoxy_{(C≤8)}, cycloalkoxy_{(C≤8)}, substituted cycloalkoxy_{(C≤8)}, alkenyloxy_{(C≤8)}, substituted alkenyloxy_{(C≤8)}, aryloxy_{(C≤8)}, substituted aryloxy_{(C≤8)}, aralkyloxy_{(C≤8)}, substituted aralkyloxy_{(C≤8)}, alkylamino_{(C≤8)}, substituted alkylamino_{(C≤8)}, dialkylamino_{(C≤8)}, substituted dialkylamino_{(C≤8)}, cycloalkylamino_{(C≤8)}, substituted cycloalkylamino_{(C≤8)}, alkenylamino_{(C≤8)}, substituted alkenylamino_{(C≤8)}, arylamino_{(C≤8)}, substituted arylamino_{(C≤8)}, aralkylamino_{(C≤8)}, substituted aralkylamino_{(C≤8)}, or a substituent convertible in vivo to hydroxy;
R₁₀ and R₁₁ are each independently hydrogen or halo;
or a pharmaceutically acceptable salt thereof.

2. The compound of claim 1, wherein the compound is further defined as: 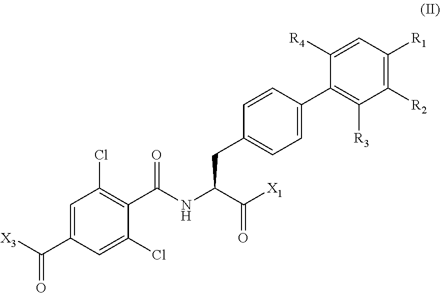
wherein:
R₁ is hydrogen, aminocarbonyl, carboxy, cyano, halo, hydroxy, —SF₅, alkyl_{(C≤8)}, substituted alkyl_{(C≤8)}, alkoxy_{(C≤8)}, substituted alkoxy_{(C≤8)}, or —Y₁—Rₐ; wherein:
Y₁ is alkanediyl_{(C≤8)} or substituted alkanediyl_{(C≤8)}; and
Rₐ is alkoxy_{(C≤12)}, acyloxy_{(C≤12)}, or a substituted version of either of these groups; or
—X(CH₂O)ₘ—(CH₂CH₂O)ₙ—R_{b}; wherein:
X is a covalent bond or —O—;
m is 0 or 1;
n is 1, 2, 3, 4, 5, 6, 7, or 8; and
R_{b} is hydrogen, alkyl_{(C≤8)}, or substituted alkyl_{(C≤8)}; or
—CH₂—O—(CH₂)ₙ—R₀; wherein:
N is 0, 1, or 2; and
R_{c} is a cyclic aliphatic ring of 4-6 in size having one oxygen as part of the ring;
R₂ is hydrogen, aminocarbonyl, carboxy, cyano, halo, hydroxy, —SF₅, alkyl_{(C≤8)}, substituted alkyl_{(C≤8)}, alkoxy_{(C≤8)}, substituted alkoxy_{(C≤8)}, or —Y₂—R₀; wherein:
Y₂ is alkanediyl_{(C≤8)} or substituted alkanediyl_{(C≤8)}; and
R_{c} is alkoxy_{(C≤12)}, acyloxy_{(C≤12)}, or a substituted version of either of these groups;
X₁ and X₃ are each independently hydroxy, alkoxy_{(C≤8)}, substituted alkoxy_{(C≤8)}, cycloalkoxy_{(C≤8)}, substituted cycloalkoxy_{(C≤8)}, alkenyloxy_{(C≤8)}, substituted alkenyloxy_{(C≤8)}, aryloxy_{(C≤8)}, substituted aryloxy_{(C≤8)}, aralkoxy_{(C≤8)}, substituted aralkoxy_{(C≤8)}, or a substituent convertible in vivo to hydroxy; and
R₃ and R₄ are each independently alkoxy_{(C≤8)} or substituted alkoxy_{(C≤8)};
provided that R₁ and R₂ are not both hydrogen;
or a pharmaceutically acceptable salt thereof.

3. The compound of claim 2, wherein the compound is further defined as: 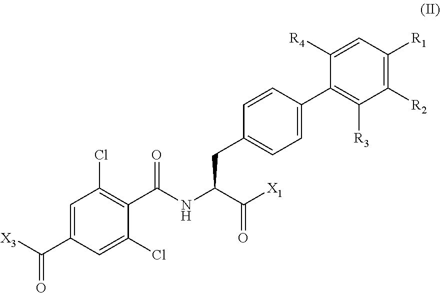
wherein:
R₁ is aminocarbonyl, carboxy, cyano, halo, hydroxy, —SF₅, alkyl_{(C≤8)}, substituted alkyl_{(C≤8)}, alkoxy_{(C≤8)}, substituted alkoxy_{(C≤8)}, or —Y₁—Rₐ; wherein:
Y₁ is alkanediyl_{(C≤8)} or substituted alkanediyl_{(C≤8)}; and
Rₐ is alkoxy_{(C≤12)}, acyloxy_{(C≤12)}, or a substituted version of either of these groups; or
—X(CH₂O)ₘ—(CH₂CH₂O)ₙ—R_{b}; wherein:
X is a covalent bond or —O—;
m is 0 or 1;
n is 1, 2, 3, 4, 5, 6, 7, or 8; and
R_{b} is hydrogen, alkyl_{(C≤8)}, or substituted alkyl_{(C≤8)}; or
—CH₂—O—(CH₂)ₙ—R_{c}; wherein:
N is 0, 1, or 2; and
R_{c} is a cyclic aliphatic ring of 4-6 in size having one oxygen as part of the ring;
R₂ is hydrogen, aminocarbonyl, carboxy, cyano, halo, hydroxy, —SF₅, alkyl_{(C≤8)}, substituted alkyl_{(C≤8)}, alkoxy_{(C≤8)}, substituted alkoxy_{(C≤8)}, or —Y₂—R_{c}; wherein:
Y₂ is alkanediyl_{(C≤8)} or substituted alkanediyl_{(C≤8)}; and
R_{c} is alkoxy_{(C≤12)}, acyloxy_{(C≤12)}, or a substituted version of either of these groups;
X₁ and X₃ are each independently hydroxy, alkoxy_{(C≤8)}, substituted alkoxy_{(C≤8)}, cycloalkoxy_{(C≤8)}, substituted cycloalkoxy_{(C≤8)}, alkenyloxy_{(C≤8)}, substituted alkenyloxy_{(C≤8)}, aryloxy_{(C≤8)}, substituted aryloxy_{(C≤8)}, aralkoxy_{(C≤8)}, substituted aralkoxy_{(C≤8)}, or a substituent convertible in vivo to hydroxy; and
R₃ and R₄ are each independently alkoxy_{(C≤8)} or substituted alkoxy_{(C≤8)};
or a pharmaceutically acceptable salt thereof.

4. The compound of claim 2, wherein the compound is further defined as: 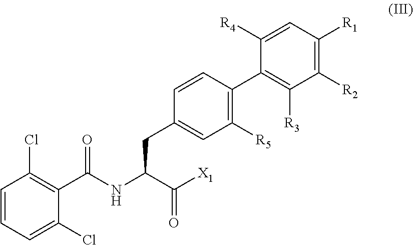
wherein:
R₁ is aminocarbonyl, carboxy, cyano, halo, hydroxy, —SF₅, alkyl_{(C≤8)}, substituted alkyl_{(C≤8)}, alkoxy_{(C≤8)}, substituted alkoxy_{(C≤8)}, or —Y₁—Rₐ; wherein:
Y₁ is alkanediyl_{(C≤8)} or substituted alkanediyl_{(C≤8)}; and
Rₐ is alkoxy_{(C≤12)}, acyloxy_{(C≤12)}, or a substituted version of either of these groups; or
—X(CH₂O)ₘ—(CH₂CH₂O)ₙ—R_{b}; wherein:
X is a covalent bond or —O—;
m is 0 or 1;
n is 1, 2, 3, 4, 5, 6, 7, or 8; and
R_{b} is hydrogen, alkyl_{(C≤8)}, or substituted alkyl_{(C≤8)}; or
—CH₂—O—(CH₂)ₙ—R_{c}; wherein:
N is 0, 1, or 2; and
R_{c} is a cyclic aliphatic ring of 4-6 in size having one oxygen as part of the ring;
R₂ is hydrogen, aminocarbonyl, carboxy, cyano, halo, hydroxy, —SF₅, alkyl_{(C≤8)}, substituted alkyl_{(C≤8)}, alkoxy_{(C≤8)}, substituted alkoxy_{(C≤8)}, or —Y₂—R_{c}; wherein:
Y₂ is alkanediyl_{(C≤8)} or substituted alkanediyl_{(C≤8)}; and
R_{c} is alkoxy_{(C≤12)}, acyloxy_{(C≤12)}, or a substituted version of either of these groups;
X₁ is hydroxy, alkoxy_{(C≤8)}, substituted alkoxy_{(C≤8)}, cycloalkoxy_{(C≤8)}, substituted cycloalkoxy_{(C≤8)}, alkenyloxy_{(C≤8)}, substituted alkenyloxy_{(C≤8)}, aryloxy_{(C≤8)}, substituted aryloxy_{(C≤8)}, aralkoxy_{(C≤8)}, substituted aralkoxy_{(C≤8)}, or a substituent convertible in vivo to hydroxy; and
R₃ and R₄ are each independently alkoxy_{(C≤8)} or substituted alkoxy_{(C≤8)};
R₅ is hydrogen, —CH(OR_{d})Rₑ, or —C(O)R_{f}, wherein:
R_{d} is hydrogen, alkyl_{(C≤8)}, substituted alkyl_{(C≤8)}, acyl_{(C≤8)}, or substituted acyl_{(C≤8)}; and
Rₑ and R_{f} are each independently alkyl_{(C≤8)} or substituted alkyl_{(C≤8)}; and
or a pharmaceutically acceptable salt thereof.

5. The compound of claim 1, wherein the compound is further defined as: 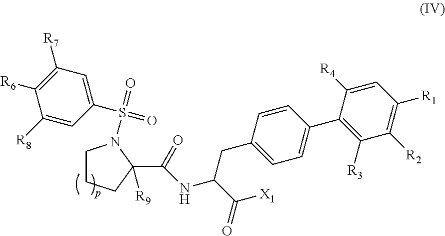
wherein:
R₁ is hydrogen, aminocarbonyl, carboxy, cyano, halo, hydroxy, —SF₅, alkyl_{(C≤8)}, substituted alkyl_{(C≤8)}, alkoxy_{(C≤8)}, substituted alkoxy_{(C≤8)}, or —Y₁—Rₐ, wherein:
Y₁ is alkanediyl_{(C≤8)} or substituted alkanediyl_{(C≤8)}; and
Rₐ is alkoxy_{(C≤12)}, acyloxy_{(C≤12)}, or a substituted version of either group; or
—X(CH₂O)ₘ—(CH₂CH₂O)ₙ—R_{b}, wherein:
X is a covalent bond or —O—;
m is 0 or 1;
n is 1, 2, 3, 4, 5, 6, 7, or 8; and
R_{b} is hydrogen, alkyl_{(C≤8)}, or substituted alkyl_{(C≤8)}; or
—CH₂—O—(CH₂)ₙ—R_{c}; wherein:
N is 0, 1, or 2; and
R_{c} is a cyclic aliphatic ring of 4-6 in size having one oxygen as part of the ring;
R₂ is hydrogen, aminocarbonyl, carboxy, cyano, halo, hydroxy, —SF₅, alkyl_{(C≤8)}, substituted alkyl_{(C≤8)}, alkoxy_{(C≤8)}, substituted alkoxy_{(C≤8)}, or —Y₂—R_{c}, wherein:
Y₂ is alkanediyl_{(C≤8)} or substituted alkanediyl_{(C≤8)}; and
R_{c} is alkoxy_{(C≤12)}, acyloxy_{(C≤12)}, or a substituted version of either group; or
R₃ and R₄ are each independently alkoxy_{(C≤8)} or substituted alkoxy_{(C≤8)};
R₆ is hydrogen or —C(O)X₂;
R₇ and R₈ are each independently hydrogen, halo, haloalkyl_{(C≤8)}, or substituted haloalkyl_{(C≤8)};
R₉ is hydrogen, alkyl_{(C≤8)}, or substituted alkyl_{(C≤8)};
p is 0, 1, 2, or 3; and
X₁ and X₂ are each independently hydroxy, alkoxy_{(C≤8)}, substituted alkoxy_{(C≤8)}, cycloalkoxy_{(C≤8)}, substituted cycloalkoxy_{(C≤8)}, alkenyloxy_{(C≤8)}, substituted alkenyloxy_{(C≤8)}, aryloxy_{(C≤8)}, substituted aryloxy_{(C≤8)}, aralkyloxy_{(C≤8)}, substituted aralkyloxy_{(C≤8)}, or a substituent convertible in vivo to hydroxy;
provided that R₁ and R₆ are not both hydrogen;
or a pharmaceutically acceptable salt thereof.

6. The compound of claim 1, wherein the compound is further defined as: 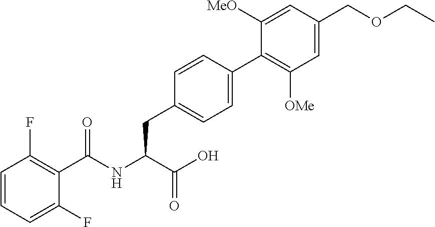 [SMILES: CCOCc1cc(OC)c(-c2ccc(C[C@H](NC(=O)c3c(F)cccc3F)C(=O)O)cc2)c(OC)c1]
or a pharmaceutically acceptable salt thereof.

7. The compound of claim 1, wherein the compound is further defined as: 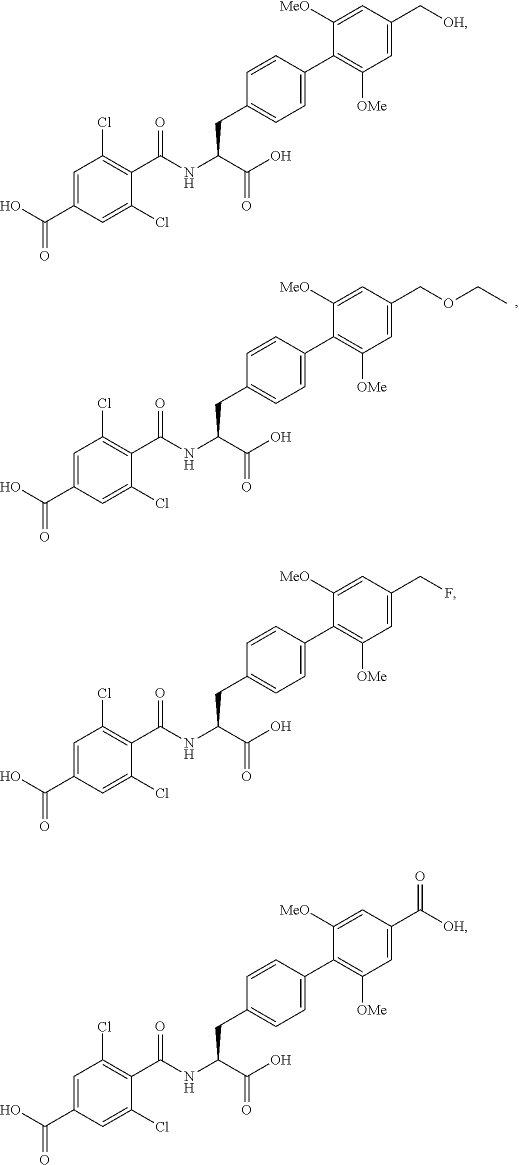 [SMILES: COc1cc(CO)cc(OC)c1-c1ccc(C[C@H](NC(=O)c2c(Cl)cc(C(=O)O)cc2Cl)C(=O)O)cc1 ; CCOCc1cc(OC)c(-c2ccc(C[C@H](NC(=O)c3c(Cl)cc(C(=O)O)cc3Cl)C(=O)O)cc2)c(OC)c1 ; COc1cc(CF)cc(OC)c1-c1ccc(C[C@H](NC(=O)c2c(Cl)cc(C(=O)O)cc2Cl)C(=O)O)cc1 ; COc1cc(C(=O)O)cc(OC)c1-c1ccc(C[C@H](NC(=O)c2c(Cl)cc(C(=O)O)cc2Cl)C(=O)O)cc1] 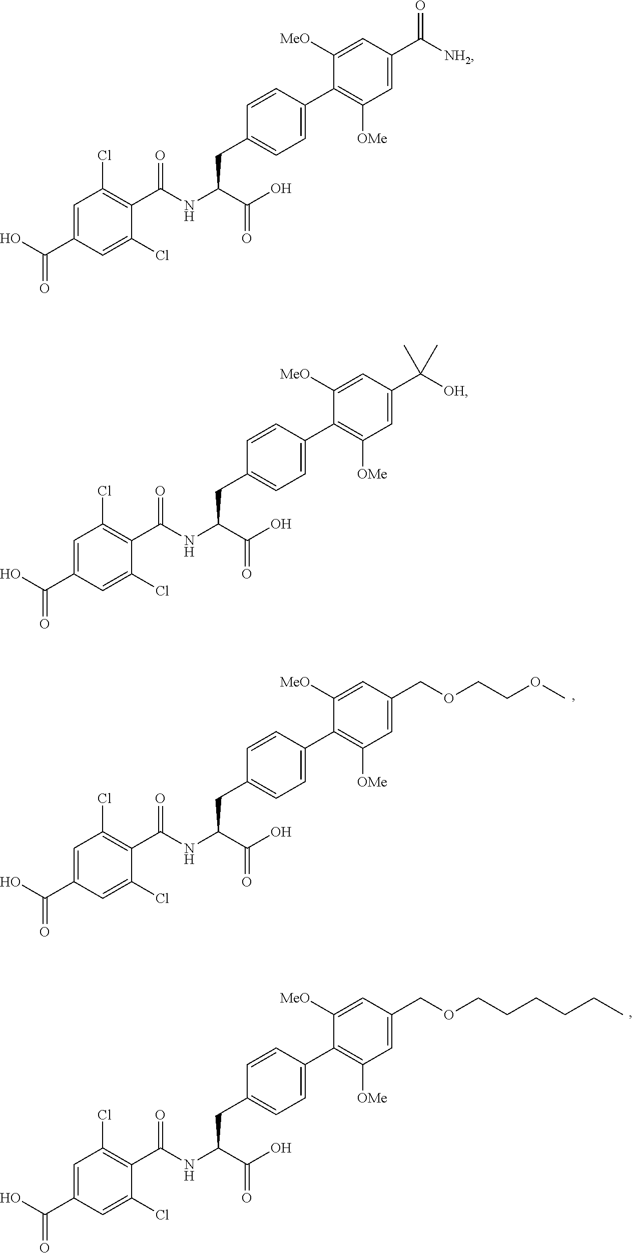 [SMILES: COc1cc(C(N)=O)cc(OC)c1-c1ccc(C[C@H](NC(=O)c2c(Cl)cc(C(=O)O)cc2Cl)C(=O)O)cc1 ; COc1cc(C(C)(C)O)cc(OC)c1-c1ccc(C[C@H](NC(=O)c2c(Cl)cc(C(=O)O)cc2Cl)C(=O)O)cc1 ; COCCOCc1cc(OC)c(-c2ccc(C[C@H](NC(=O)c3c(Cl)cc(C(=O)O)cc3Cl)C(=O)O)cc2)c(OC)c1 ; CCCCCCOCc1cc(OC)c(-c2ccc(C[C@H](NC(=O)c3c(Cl)cc(C(=O)O)cc3Cl)C(=O)O)cc2)c(OC)c1] 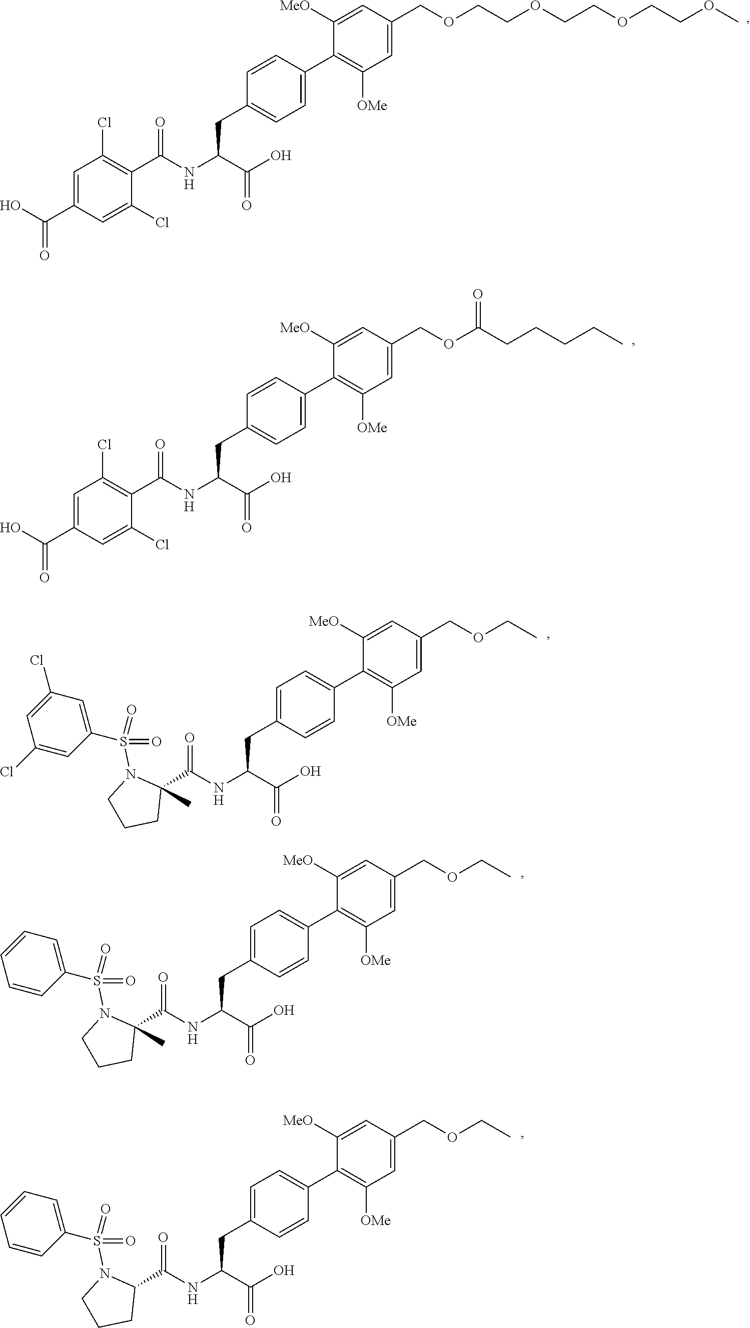 [SMILES: COCCOCCOCCOCc1cc(OC)c(-c2ccc(C[C@H](NC(=O)c3c(Cl)cc(C(=O)O)cc3Cl)C(=O)O)cc2)c(OC)c1 ; CCCCCC(=O)OCc1cc(OC)c(-c2ccc(C[C@H](NC(=O)c3c(Cl)cc(C(=O)O)cc3Cl)C(=O)O)cc2)c(OC)c1 ; CCOCc1cc(OC)c(-c2ccc(C[C@H](NC(=O)[C@]3(C)CCCN3S(=O)(=O)c3cc(Cl)cc(Cl)c3)C(=O)O)cc2)c(OC)c1 ; CCOCc1cc(OC)c(-c2ccc(C[C@H](NC(=O)[C@]3(C)CCCN3S(=O)(=O)c3ccccc3)C(=O)O)cc2)c(OC)c1 ; CCOCc1cc(OC)c(-c2ccc(C[C@H](NC(=O)[C@@H]3CCCN3S(=O)(=O)c3ccccc3)C(=O)O)cc2)c(OC)c1] 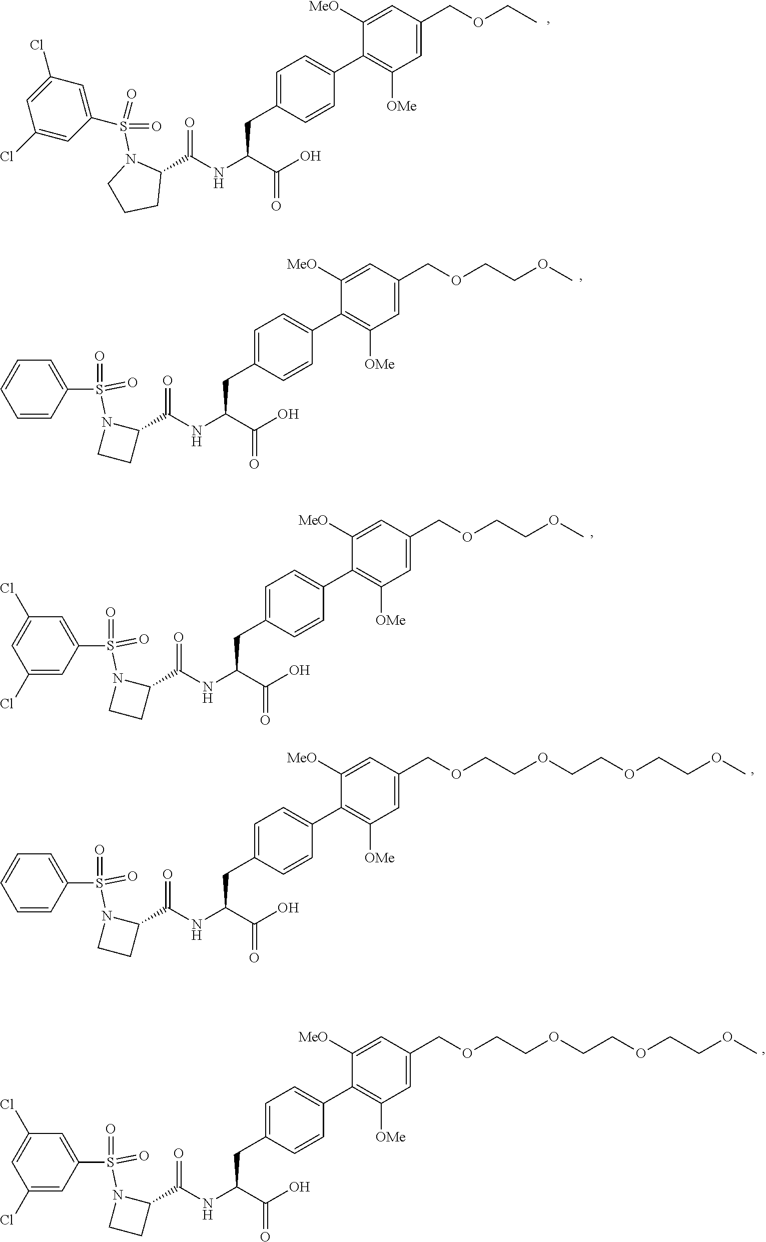 [SMILES: CCOCc1cc(OC)c(-c2ccc(C[C@H](NC(=O)[C@@H]3CCCN3S(=O)(=O)c3cc(Cl)cc(Cl)c3)C(=O)O)cc2)c(OC)c1 ; COCCOCc1cc(OC)c(-c2ccc(C[C@H](NC(=O)[C@@H]3CCN3S(=O)(=O)c3ccccc3)C(=O)O)cc2)c(OC)c1 ; COCCOCc1cc(OC)c(-c2ccc(C[C@H](NC(=O)[C@@H]3CCN3S(=O)(=O)c3cc(Cl)cc(Cl)c3)C(=O)O)cc2)c(OC)c1 ; COCCOCCOCCOCc1cc(OC)c(-c2ccc(C[C@H](NC(=O)[C@@H]3CCN3S(=O)(=O)c3ccccc3)C(=O)O)cc2)c(OC)c1 ; COCCOCCOCCOCc1cc(OC)c(-c2ccc(C[C@H](NC(=O)[C@@H]3CCN3S(=O)(=O)c3cc(Cl)cc(Cl)c3)C(=O)O)cc2)c(OC)c1] 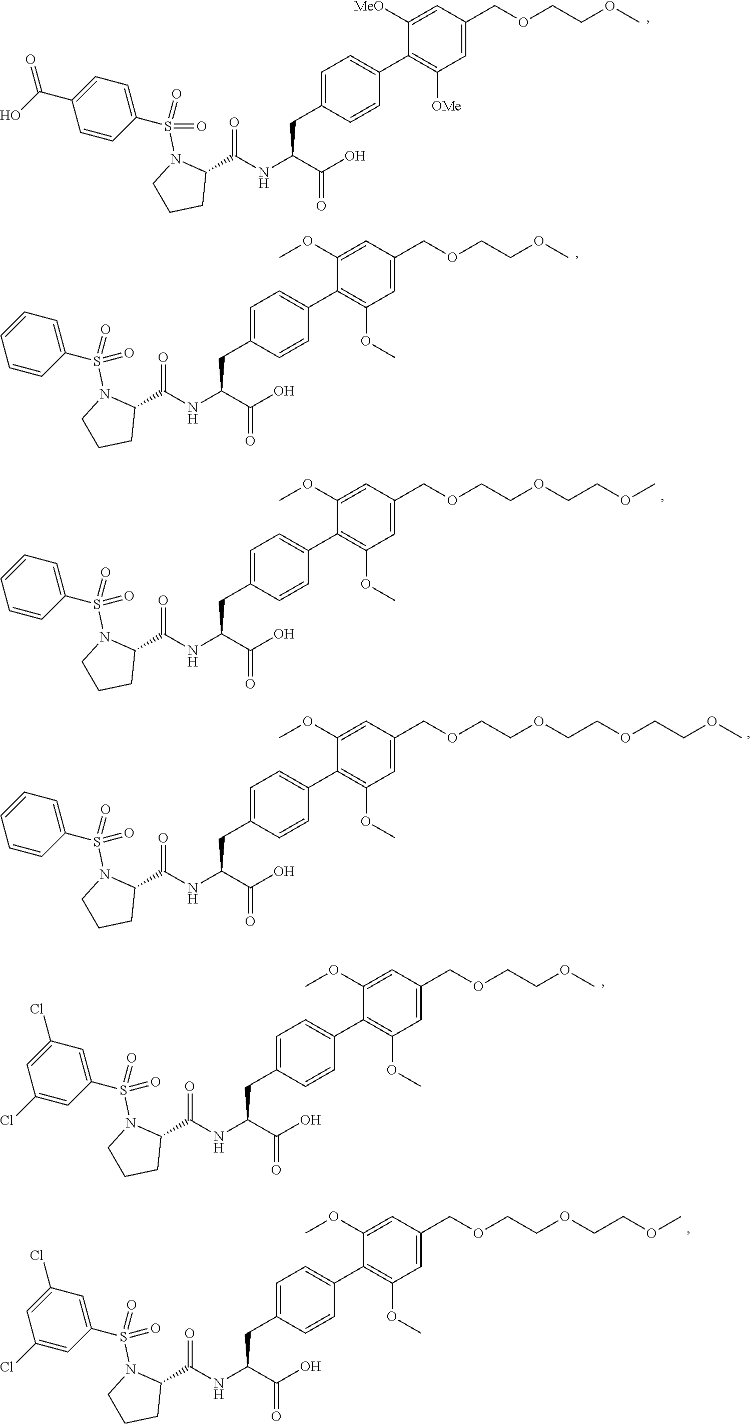 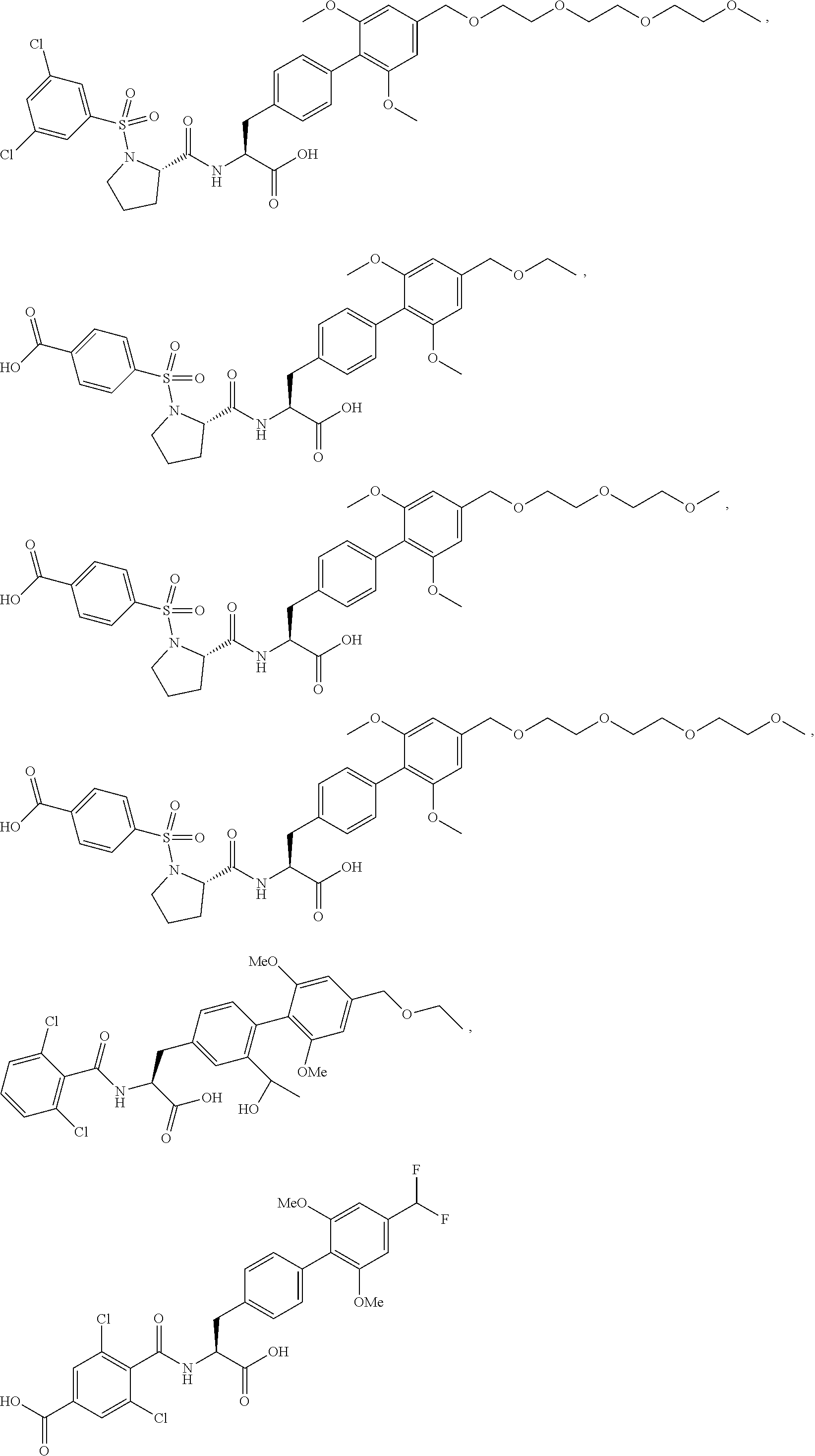 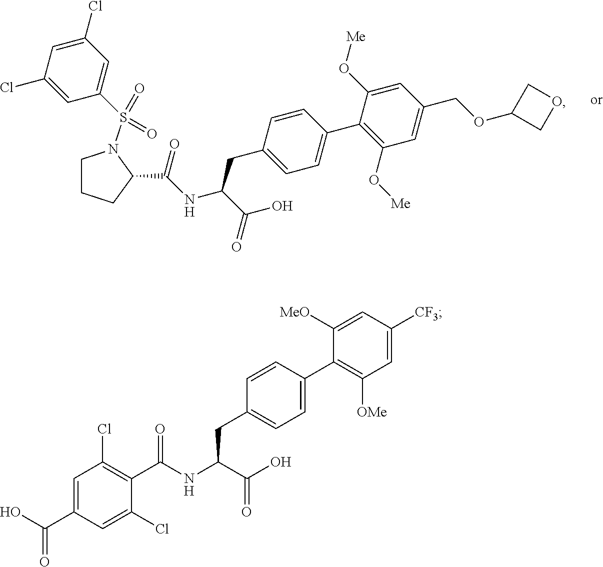 [SMILES: COc1cc(COC2COC2)cc(OC)c1-c1ccc(C[C@H](NC(=O)[C@@H]2CCCN2S(=O)(=O)c2cc(Cl)cc(Cl)c2)C(=O)O)cc1 ; "or" ; COc1cc(C(F)(F)F)cc(OC)c1-c1ccc(C[C@H](NC(=O)c2c(Cl)cc(C(=O)O)cc2Cl)C(=O)O)cc1]
or a pharmaceutically salt thereof.

8. A composition comprising:
(A) a VLA-4 inhibitor; and
(B) an agent which interacts with one or more chemokine receptors.

9. The composition according to claim 8, wherein the agent which interacts with a chemokine receptor is an agent which interacts with a C—X—C chemokine receptor.

10. The composition of claim 9, wherein the agent is a CXCR2 agonist.

11. The composition according to claim 8, wherein the agent is plerixafor, Groβ, or a derivative of Groβ.

12. The composition of claim 11, wherein the derivative of Groβ is a truncated Groβ.

13. The composition of claim 12, wherein the truncated Groβ is SB-251353.

14. The composition according to claim 8, wherein the composition further comprises an inhibitor of integrin α9β1, G-CSF, a derivative of G-CSF, or a combination thereof.

15. A composition comprising:
a) a VLA-4 inhibitor compound;
b) a first agent which interacts with one or more chemokines; and
c) a second agent which interact with one or more chemokines.

16. The composition of claim 15, wherein the first agent and second agent which interact with a chemokine are selected from the group consisting of: an agent which interacts with a C—X—C chemokine or a C—X—C chemokine receptor.

17. The composition of claim 16, wherein the first agent is a CXCR2 agonist.

18. The composition of claim 15, wherein the second agent is a CXCR4 inhibitor.

19. The composition of according to claim 15, wherein the first agent is Gro-β, or a derivative of Gro-β.

20. The composition of claim 18, wherein the CXCR4 inhibitor is one or more of AMD3100 (plerixafor), AMD3465, CTCE-0214, CTCE-9908, CP-1221 (linear peptides, cyclic peptides, natural amino-acids, unnatural amino acids, and peptidomimetic compounds), T140 and analogs, 4F-benzoyl-TN24003, KRH-1120, KRH-1636, KRH-2731, polyphemusin analogue, ALX40-4C, or combinations thereof.
